(19) United States
(12) Patent Application Publication
(10) Pub. No.: US 2022/0372138 A1
(43) Pub. Date: Nov. 24, 2022

(54) ANTIBODIES BINDING IGC2 OF IGSF11 (VSIG3) AND USES THEREOF

(71) Applicant: IOMX THERAPEUTICS AG, Martinsried (DE)

(72) Inventors: Maximilian AIGNER, Martinsried (DE); Stefan BISSINGER, Martinsried (DE); Simone BRAENDLE, Martinsried (DE); Sabrina GENSSLER, Martinsried (DE); Stefanie URLINGER, Martinsried (DE); Nisit KHANDELWAL, Martinsried (DE); Jörg Thomas REGULA, Martinsried (DE); Tillmann MICHELS, Martinsried (DE); Jonas ZANTOW, Martinsried (DE)

(21) Appl. No.: 17/624,447

(22) PCT Filed: Jul. 6, 2020
(86) PCT No.: PCT/EP2020/069014

§ 371 Date: Jan. 3, 2022

(30) Foreign Application Priority Data
Jul. 5, 2019 (EP) 19184708.6

(51) Int. Cl. C07K 16/28; A61P 35/00; G01N 33/68; G01N 33/50

(52) U.S. Cl. CPC C07K 16/2803; A61P 35/00; G01N 33/6854; G01N 33/5011; A61K 39/00

(57) ABSTRACT

The invention is based on the surprising finding of antibodies that bind to an immunoglobulin-like (Ig) domain of the extra cellular domain (ECD) of IGSF11 (VSIG3) can also inhibit the interaction between IGSF11 and IGSF11 receptors such as VSIR (VISTA), the inhibition of such interaction can sensitise tumour cells to anti-tumour immune responses. In particular, the invention provides products, compositions and methods for treating diseases using modulators of IGSF11, especially antigen binding proteins targeting an Ig domain of IGSF11-ECD, including those being inhibitors of IGSF11-interaction with VSIR. Also provided are methods of sensitising cells involved with a proliferative disorder against the cytotoxic effect of cell-mediated immune responses, and/or to kill such cells and/or methods for treating proliferative diseases, using an IGSF11 inhibitor such as an antibody binding to an Ig domain of IGSF11-ECD, as well as certain related aspects including detection, diagnostic and screening methods.

The invention is based on the surprising finding of antibodies that bind to an immunoglobulin-like (Ig) domain of the extra cellular domain (ECD) of IGSF11 (VSIG3) can also inhibit the interaction between IGSF11 and IGSF11 receptors such as VSIR (VISTA), the inhibition of such interaction can sensitise tumour cells to anti-tumour immune responses. In particular, the invention provides products, compositions and methods for treating diseases using modulators of IGSF11, especially antigen binding proteins targeting an Ig domain of IGSF11-ECD, including those being inhibitors of IGSF11-interaction with VSIR. Also provided are methods of sensitising cells involved with a proliferative disorder against the cytotoxic effect of cell-mediated immune responses, and/or to kill such cells and/or methods for treating proliferative diseases, using an IGSF11 inhibitor such as an antibody binding to an Ig domain of IGSF11-ECD, as well as certain related aspects including detection, diagnostic and screening methods.

In the treatment of cancer there are a number of approaches by which therapies may lead to the elimination of tumour cells, including those that involve or exploit one or more components of the immune system, either directly or indirectly. One of the limitations associated with such therapies is that cancerous cells often exploit immune-checkpoints to evade a patient's immune system, such as by preventing immune-recognition or down-regulating a tumour-specific cytotoxic T cell (CTL) response, thereby generating resistance against an immune response (Rabinovich et al 2007, Annu Rev Immunol 25:267; Zitvogel et al 2006, Nat Rev Immunol 6:715). Under normal conditions, such immune-regulatory checkpoints are crucial for the maintenance of self-tolerance under physiological conditions, but there is an increasing recognition of the important role that they can also play in cancer (Hanahan and Weinberg 2011, Cell; 144:646); cancerous cells can take over these mechanisms to evade and suppress the immune system in order to develop into a tumour (Drake et al 2006, Adv Immunol 90:51).

Current state of the art cancer therapies include blockade of those few immune-regulatory checkpoints presently known and for which their mechanism of action is understood. For example, blocking antibodies against surface-expressed immune-regulatory proteins, such as CTLA4 and PD-L1 (Chambers et al 2001, Annu Rev Immunol 19:565; Blank et al 2004, Cancer Res 64:1140), can boost anti-tumour immunity and have shown clinical success against many cancer types (Page et al 2014, Annu Rev Med 65:185). However, a large proportion of cancer patients does not respond to such checkpoint blockage therapy (Bu et al 2016, Trends Mol Med 22:448; Hugo et al 2016, Cell 165:35; Topalian et al 2012, New Engl J Med 366:2443), indicating that other immune-checkpoint pathways may be active. Indeed, synergistic cooperation between several immune-regulatory pathways maintains immune tolerance against tumours, which might explain why blocking only one immune-regulatory checkpoint node can still result in tumour escape (Woo et al 2012, Cancer Res 72:917; Berrien-Elliott et al 2013, Cancer Res 73:605). However, little is known about the molecular factors that are central to the mechanism of action of such immune-regulatory pathways. Indeed, successful cancer immunotherapy requires a systematic delineation of the entire immune-regulatory circuit—the ‘immune modulatome’—expressed by tumours. Therefore, today, there is still an unmet need for identifying further molecular targets that may serve as immune-regulatory checkpoints and in particular an unmet need for means and methods to modulate, detect and otherwise utilise such possible checkpoint targets, such as in medicine, diagnosis and research.

V-set immunoregulatory receptor (VSIR), initially described and designated as “V-domain Ig suppressor of T cell activation” (VISTA) by Wang et al (2011; J Exp Med 208:777), is an immunoglobulin (Ig) superfamily ligand that negatively regulates T cell responses. VISTA is primarily expressed on hematopoietic cells, and VISTA expression is highly regulated on myeloid antigen-presenting cells (APCs) and T cells. Wang et al described that a soluble VISTA-Ig fusion protein or VISTA expression on APCs inhibited T cell proliferation and cytokine production in vitro, and that a VISTA-specific monoclonal antibody interfered with VISTA-induced suppression of T cell responses by VISTA-expressing APCs in vitro. These findings showed that VISTA had functional activities that were non-redundant with other Ig superfamily members, and Wang et al postulated further that VISTA might play a role in the development of autoimmunity and immune surveillance in cancer.

VISTA is since known as a broad-spectrum negative checkpoint regulator for cancer immunotherapy (Lines et al, 2014; Cancer Immunol Res 2:510). For example, initial studies described VISTA as a potent negative regulator of T-cell function that is expressed on hematopoietic cells. VISTA levels are heightened within the tumour microenvironment (TME), in which its blockade can enhance anti-tumour immune responses in mice. Results have established VISTA as a negative checkpoint regulator that suppresses T-cell activation, that induces Foxp3 expression, and is highly expressed within the tumour microenvironment, leading to the suggestion that VISTA blockade may offer an immunotherapeutic strategy for human cancer (Lines et al, 2014; Cancer Res 74:1924).

Indeed, VISTA blockade has been shown to impair the suppressive function and reduce the emergence of tumour-specific Foxp3+CD4+ regulatory T cells. Consequently, VISTA mAb administration as a monotherapy significantly suppressed the growth of both transplantable and inducible melanoma. Initial studies exploring a combinatorial regimen using VISTA blockade and a peptide-based cancer vaccine with TLR agonists as adjuvants suggested that VISTA blockade synergised with the vaccine to effectively impair the growth of established tumours. These studies thereby established a foundation for designing VISTA-targeted approaches either as a monotherapy or in combination with additional immune-targeted strategies for cancer immunotherapy (Le Mercier et al, 2014; Cancer Res 74:1933).

Subsequently, VISTA has been associated with acquired resistance to anti-PD-1 therapy in metastatic melanoma patients (Kakavand et al, 2017; Modern Pathol 89, doi:10.1038/modpathol.2017.89; published online 4 Aug. 2017), and as a compensatory inhibitory pathway in prostate tumours after ipilimumab therapy (Gao et al, 2017; Nat Med 23:551). Furthermore, the immune-checkpoint protein VISTA has been described to critically regulate the IL-23/IL-17 inflammatory axis (Li et al, 2017; Sci Rep 7:1485).

WO2016/090347 describes V-set and immunoglobulin domain-containing protein 8 (VSIG8) as the receptor for VISTA, as well as the use of VSIG8 in the identification or synthesis of agonist or antagonist compounds, preferably antibodies, polypeptides and fusion proteins which agonise or antagonise the effects of VSIG8 and/or VISTA and/or the VSIG8/VISTA binding interaction. Such VSIG8 antagonists were postulated therein to be used to suppress VISTA's suppressive effects on T cell immunity, and more particularly used in the treatment of cancer, or infectious disease; and such agonist compounds were postulated therein to be used to potentiate or enhance VISTA's suppressive effects on T cell immunity and thereby suppress T cell immunity, such as in the treatment of autoimmunity, allergy or inflammatory conditions. Screening assays for identifying agonists and antagonist of and/or VISTA and/or the VSIG8/VISTA binding interaction compounds were also described in WO2016/090347.

Johnston et al recently described P-selectin glycoprotein ligand-1 (PSGL-1) as an independent ligand for VISTA under acidic pH conditions (Johnston et al 2019, Nature 574:565). VISTA-specific antibodies that were engineered to selectively bind and block this interaction under acidic conditions were shown to rescue immune suppression in vitro and in vivo. Furthermore, WO2019/165233 discloses the interaction of VISTA with Leucine Rich Repeats And Immunoglobulin Like Domains 1 (LRIG1). LRIG1 is a transmembrane protein that has been shown to negatively regulate signalling by receptor tyrosine kinases. WO2019/165233 discloses LRIG1-binding antibodies that disrupt the interaction with VISTA and mediate anti-tumour activity in xenograft mouse model. WO2015/179799 also postulates a homophilic interaction between VISTA and VISTA.

The Ig-superfold of immunoglobulin superfamily proteins is characterized by a primary sequence motif that spans some 100 amino acids. In three dimensions, this sequence motif translates into a compact domain structure that comprised of two anti-parallel beta-sheets packed face to face. Although there is a defined topology and connectivity for the Ig-superfold, the number of beta-strands is variable. To take account of this variability Ig-like domains have been classified into different sets, according to the number and arrangement of the beta-strands. The nomenclature is standardised with the beta-strands labelled sequentially from A to G, and structurally equivalent beta-strands in different sets retain the same letter. The I set is defined as having strands ABED in one beta-sheet and A‘GFCC’ in the other. The V set has an extra C-delta strand in the latter beta-sheet, while sets C1 and C2 lack strands A′, and A′, and D, respectively.

The key role of the front face of immunoglobulin-like V-type domains (in particular, the GFC, CFCC′ or AGFCC′ Ig beta-sandwich front face) in interactions between immunoglobulin superfamily members is generally understood in the art, and such GFC face-mediated Ig domain interactions are the most common way for Ig domains to bind, and have been captured by X-ray crystallography, in nearly every minimal binding complex between cell surface immunoregulatory receptors (Stengel et al 2012, PNAS 109:5399), and even antibodies and T-cell receptor (TCR) complexes (Lin et al, 2008; PNAS 105:3011). Indeed, the role of V-type domains in intercellular binding between immunoglobulin superfamily receptor/ligand pairs has been generally accepted and widely described, including for several immunoglobulin superfamily receptor/ligand pairs involved in tumour cell immune evasion, such as: (i) PD1 interacting with PDL1 or PDL2 (eg, Lin et al 2008; Lazar-Molnar et al 2009, PNAS 105:10483); (ii) CD80 interacting with CD28 or CTLA4 (eg, Sanchez-Lockhart et al 2014, PLoS One 9:e89263; Stamper et al 2001, Nature 410:608); and (iii) CD86 interacting with CD28 or CTLA4 (eg, Rennert et al 1997, Int Immunol 9:805).

WO2018/027042 (Bio-Techne Corp) discloses antibodies binding to the IgV domain of IGSF11 (VSIG3), and WO2019/152810 discloses antibodies that bind to recombinant human IGSF11 (VISIG3) and that modulate the interaction of VISTA and recombinant human VSIG3. Such documents do not include a showing of in-vivo anti-tumour activity of the antibodies disclosed therein; in particular, not for those that bind to a specific domain of recombinant IGSF11 and the association between such binding-domain, modulation of the interaction of between VISTA and recombinant VSIG3 and in-vivo activity.

Recently, expression of IGSF11 (VISIG3) was reported to be significantly lower in squamous non-small cell lung cancer (sqNSCLC) sample with high myeloid infiltration compared to those with low myeloid infiltration (Cruzalegui et al 20020, In: Proceedings of the 111th Annual Meeting of the American Association for Cancer Research; 2020 Jun. 22-24. Philadelphia (Pa.): AACR; 2020. Abstract nr 3327). IGSF11 (VISIG3) (and PSGL1, another putative ligand of VISTA) were reported to be upregulated and frequently co-expressed with VISTA in human NSCLC, and exhibiting higher co-localisation in EGFR mutated lung adenocarcinomas, and VSIG3/VISTA (and PSGL1/VISTA) co-localisation was described to be consistently associated with better prognosis in NSCLC patients treated without immunotherapy, but with worse outcome in cases treated with PD-1 axis blockers (Ding et al 2020, In: Proceedings of the 111th Annual Meeting of the American Association for Cancer Research; 2020 Jun. 22-24. Philadelphia (Pa.): AACR; 2020. Abstract nr 5525).

Therefore, there is a need, from one or more of the above perspectives, for novel approaches to render cells involved with certain disorders (such as a tumour) more (or less) susceptible to the immune system, and in particular to circumvent tumour immune escape mechanisms. The present invention seeks to provide, in particular, novel therapeutic approaches and methods involving existing or novel compounds; for example, compounds and ABPs that sensitise such cells towards a cytotoxic response of the immune system or components thereof. Furthermore, the invention seeks to provide novel strategies to diagnose, prognose and/or monitor cell resistance to such an immune response or components, as wells as screening approaches for the identification of compounds that are useful in the treatment of certain disorders. Accordingly, it is an object of the present invention to provide alternative, improved, simpler, cheaper and/or integrated means or methods that address one or more of these or other problems. Such an object underlying the present invention is solved by the subject matter as disclosed or defined anywhere herein, for example by the subject matter of the attached claims.

The invention is grounded by the surprising finding that it is the immunoglobulin-like C2-type domain of Immunoglobulin superfamily member 11, “IGSF11” (or VSIG3) which is involved with the interaction between IGSF11 and B7 family member V-set immunoregulatory receptor, “VSIR” (which was initially described and designated as V-domain Ig suppressor of T cell activation, or VISTA), and that antibodies which bind to such immunoglobulin-like C2-type domain of IGSF11 affect the function of IGSF11 expressed on tumour cells, such as by attenuating the resistance exhibited by such cells to an immune response.

Generally, therefore, and by way of brief description, the main aspects of the present invention can be described as follows:

In one aspect, the invention relates to a method for identifying and/or characterising an ABP as one specifically binding to a C2-type immunoglobulin-like (IgC2) domain of IGSF11 (VSIG3) protein or a variant thereof, the method comprising the step of: detecting binding of the ABP to an epitope of (or comprised in) such domain of IGSF11 protein; thereby identifying and/or characterising the ABP as one that specifically binds to the IgC2 domain of IGSF11 protein (or variant thereof).

In another aspect, the invention relates to a method for identifying and/or characterising an ABP for use in medicine, the method comprising the steps of: (x) providing an ABP that binds to IGSF11 protein; and (y) identifying and/or characterising the provided ABP as one that specifically binds to an IgC2 domain of IGSF11 protein or a variant thereof, thereby identifying and/or characterising the ABP for use in medicine.

Other aspects of the present invention include uses of and various methods involving an IgC2 domain of the IGSF11 protein.

Additionally, in a first aspect directed to ABPs, the invention relates to an antigen binding protein (ABP) which specifically binds to a C2-type immunoglobulin-like (IgC2) domain of IGSF11 (VSIG3) protein and, optionally, wherein the ABP is able to inhibit the binding of an interacting protein such as VSIR (VISTA) protein or a variant thereof to IGSF11 protein or a variant thereof.

In a second aspect, the invention relates to an ABP which competes with an ABP of a first aspect for binding to an IgC2 domain of IGSF11 protein. In a related aspect, the invention relates to an ABP which binds to the same epitope as an ABP of a first aspect.

In another aspect, the invention relates to an antigen binding domain (ABD) of an ABP of the invention.

In a third aspect, the invention relates to a nucleic acid encoding for an ABP or ABD of the invention or of components thereof, and in related aspects, the invention relates to a nucleic acid construct (NAC) comprising such a nucleic acid, and relates to a host cell comprising a nucleic acid or NAC of the invention.

In a fourth aspect, the invention relates to a pharmaceutical composition comprising an ABP, ABD, nucleic acid, NAC or host cell of the invention, or comprising a compound that specifically binds to and/or is a modulator of the expression, function, activity and/or stability of an IgC2 domain of immunoglobulin superfamily member 11 (IGSF11, or VSIG3), or of a variant of such domain of IGSF11, and a pharmaceutically acceptable carrier, stabiliser and/or excipient.

In a fifth aspect, the invention relates to a method for the treatment of certain diseases, disorders or conditions in a subject by administering a product to the subject, wherein the product is selected from the list consisting of an ABP, ABD, nucleic acid, NAC and host cell of the invention, or is a compound that specifically binds to and/or is a modulator of the expression, function, activity and/or stability of an IgC2 domain of immunoglobulin superfamily member 11 (IGSF11, or VSIG3), or of a variant of such domain of IGSF11. In related aspects, the invention relates to a product for use in medicine, and relates to the use of a product for the manufacture of a medicament, wherein the product is selected from the list consisting of an ABP, ABD, nucleic acid, NAC or host cell of the invention, or is a compound that specifically binds to and/or is modulator of the expression, function, activity and/or stability of an IgC2 domain of immunoglobulin superfamily member 11 (IGSF11, or VSIG3), or of a variant of such domain of IGSF11.

The invention also relates to various methods to produce a recombinant cell line or ABP of the invention, a hybridoma or host cell capable of producing an ABP of the present invention, as well as relating to various determination and/or diagnostic methods or uses, and to kits useful for such determination and/or diagnostic methods, as well as to various methods for identifying and/or charactering compounds and/or methods for identifying, generating and/or producing ABPs, such as those suitable for use in medicine.

The figures show:

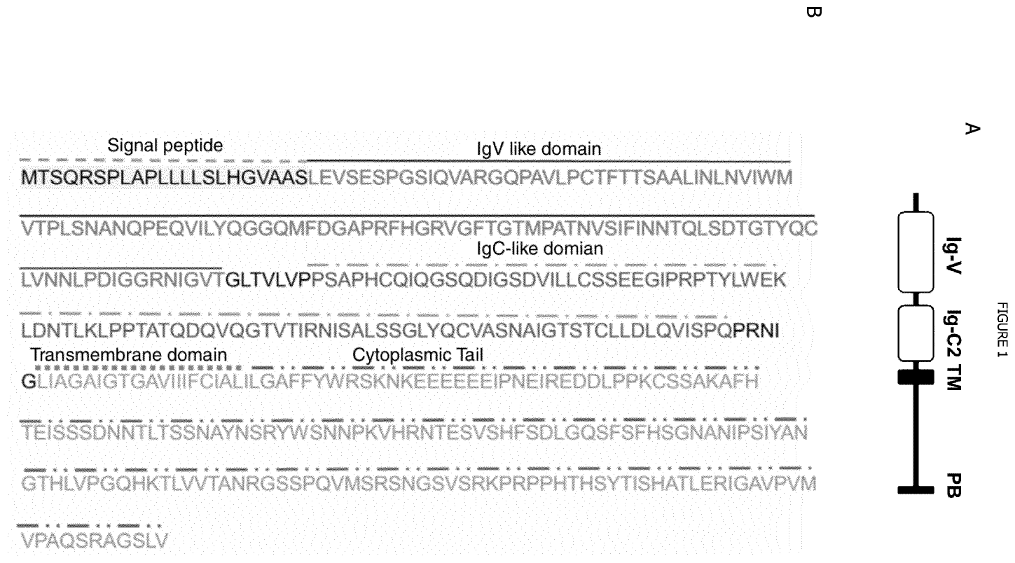

FIG. 1: A pictorial representation of the domain structure of IGSF11 (VSIG3). Ig-V/C2=immunoglobulin V/C2-like; TM=transmembrane; and PB=PDZ-binding. Adapted from Jang et al (2016; Nat Neurosci 19:84) (A). A predicted structure of human IGSF11. Signal peptide, IgV-like and IgC2-like domains, transmembrane region, and the cytoplasmic tail of human IGSF11protein are indicated (from Wang et al, 2018) (B).

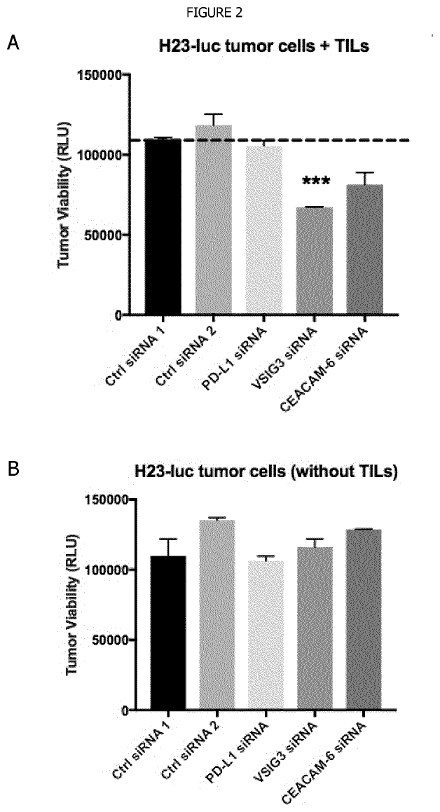

FIG. 2: IGSF11 (VSIG3) knockdown sensitises lung tumour cells towards tumour infiltrating lymphocytes (TIL)-mediated cytotoxicity. H23 NSCLC cell lines stably transfected with a pEGFP-luc reporter plasmid were treated with the noted siRNAs and then co-cultured in the presence (A) or absence (B) of patient-derived TILs before the viability of tumour cells was measured for remaining luciferase activity.

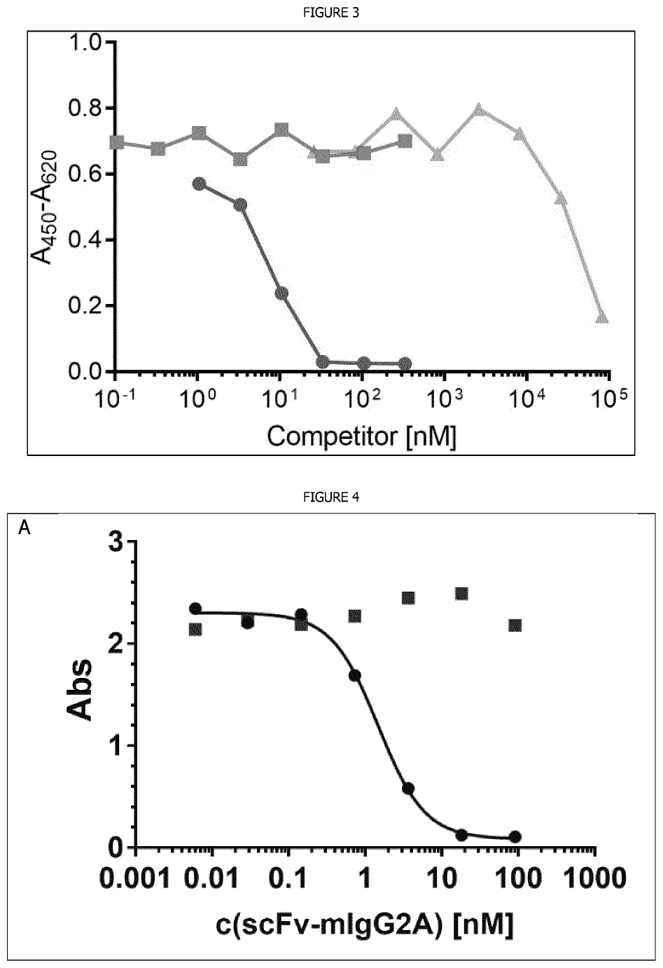

FIG. 3: ELISA assay to detect inhibition of binding between IGSF11 (VSIG3) and VSIR (VISTA). Purified and immobilised extracellular domain (ECD) of human IGSF11 (VSIG3) (HIS6-tagged) can interact with VSIR (VISTA), and this interaction can be blocked with a mouse anti-VISTA monoclonal antibody (circles) but not isotype control antibody (squares). Also, the soluble ECD of IGSF11 (triangles) can inhibit the interaction.

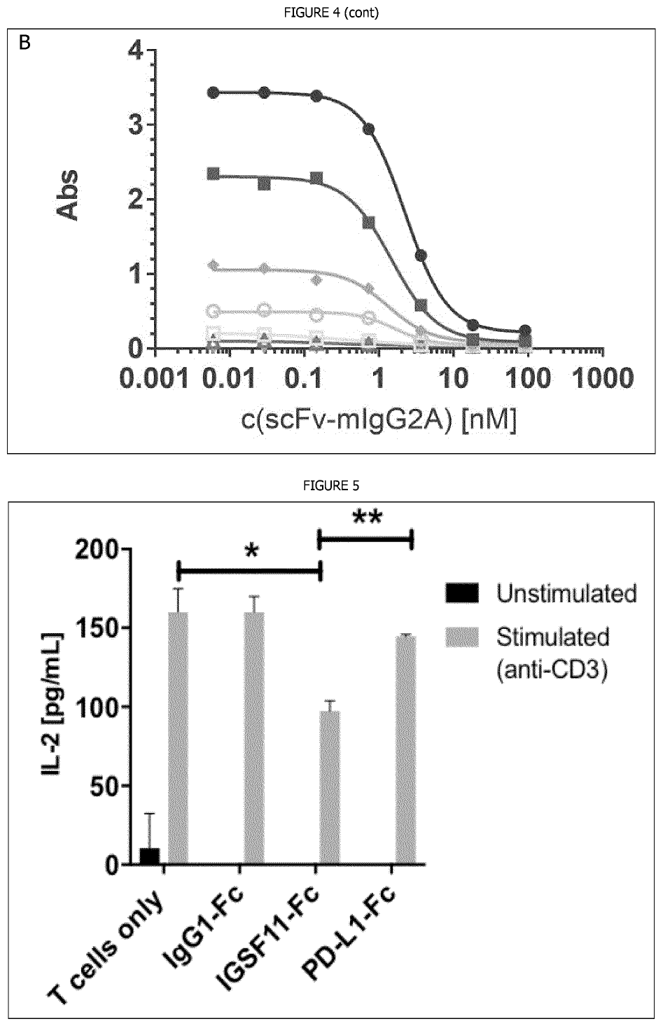

FIG. 4: scFv-Fc-format ABPs of the invention can inhibit the interaction between IGSF11 and VSIR (Fc-protein): (A) with an IC50 of less than about 1.5 nM at a VSIR-Fc concentration of 6.6 ug/mL (about 74 nM dimer concentration). Circles=scFv-Fc-format of antibody A-015 of the invention, Squares=unrelated antibody isotype control; and (B) with IC50s from about 2.2 nM to 1.6 nM at VSIR-Fc concentrations from about 20 ug/mL to 1.6 ug/mL (about from 22 nM to 8 nM dimer concentration), respectively. VSIR-Fc concentrations: solid circles=20 ug/mL, solid squares=6.66 ug/mL, solid diamonds=2.22 ug/mL, open circles=0.74 ug/mL, open squares=0.062 ug/mL and open triangles=0.027 ug/mL, corresponding to VSIR-Fc dimer concentrations or about 222 nM, 74 nM, 24.7 nM, 8.2 nM, 2.7 nM, 0.9 nM and 0.3 nM, respectively.

FIG. 5: IGSF11 (Fc protein) can inhibit the production of IL-2 by stimulated T cells. “*”=P<0.05, “*”=P<0.01.

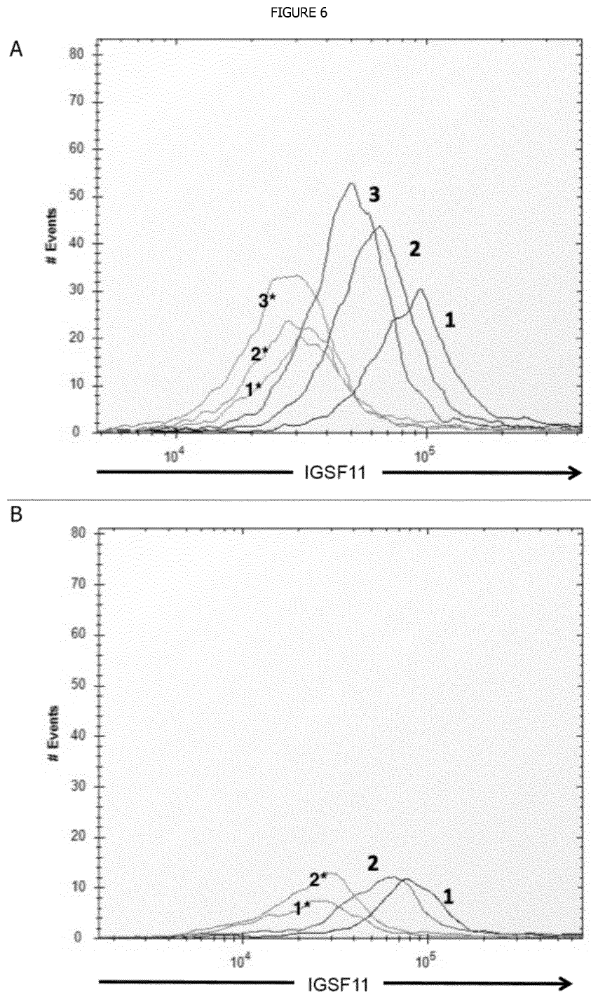

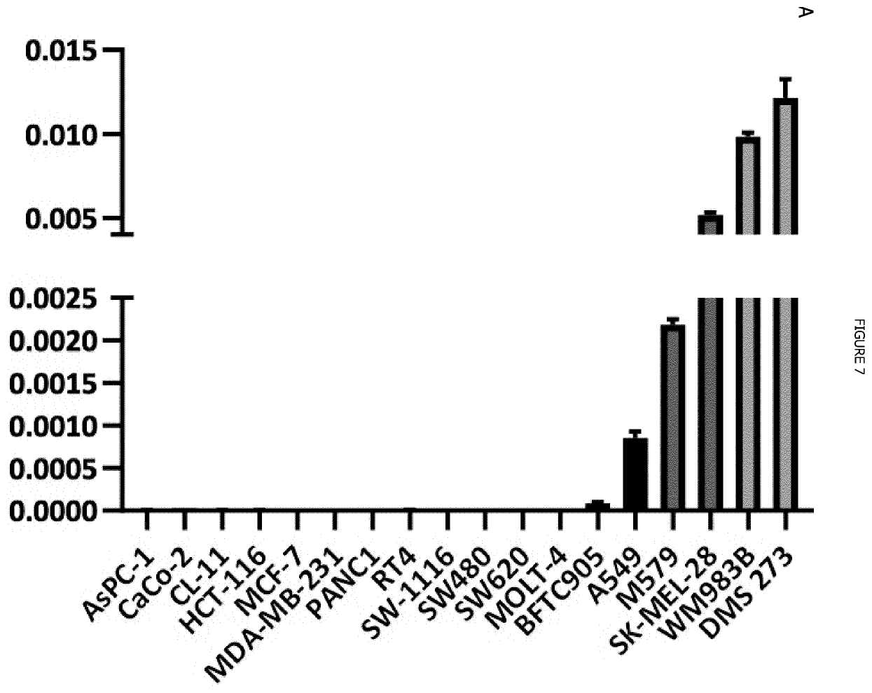

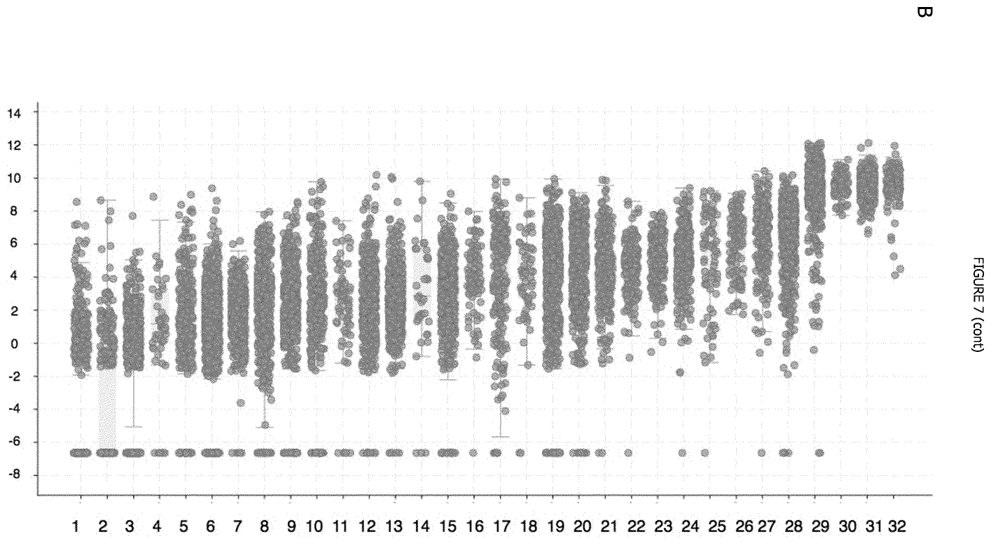

FIG. 6: IGSF11 can be detected on the surface of monocytes from PBMC of healthy volunteers. FACS histogram curves show detection of IGSF11 on monocytes of two volunteers (A and B) at the following concentrations of the anti-IGSF11 scFv-Fc format of antibody A-015: 1=150 ug/mL, 2=37.5 ug/mL, 3=9.38 ug/mL. Those marked by “*” are histogram curves from mouse IgG2a isotype control used at the corresponding concentration.

FIG. 7: IGSF11 expression across: (A) various specific cancer cell lines tested by the inventors, expression level measured using qPCR. X-axis: relative IGSF11 expression (relative to GAPDH); Y-Axes: cell line name; and (B) IGSF11 expression is noted across several tumour types in the TCGA pan-cancer genome expression database as shown by RNA expression (figure/data from the TCGA pan-cancer genome expression database, and analysed using cBioportal for Cancer Genomics: Gao et al 2013, Sci Signal 6:pl1: Cerami et al 2012, Cancer Discov 2:401). X-axis: relative IGSF11 expression—RNA Seq V2 (log 2); Y-Axes: 1=liver, 2=AML; 3=colorectal; 4=DLBC; 5=cervical; 6=breast; 7=prostate; 8=stomach; 9=lung adenoma; 10=sarcoma; 11=chromophobe; 12=ccRCC; 13=thyroid; 14=cholangiocarcinoma; 15=uterine; 16=mesothelioma; 17=oesophagus; 18=uterine CS; 19=head and neck; 20=bladder; 21=pRCC; 22=testicular germ cell; 23=pancreas; 24=ovarian; 25=ACC; 26=thymoma; 27=PCPG; 28=lung squ; 29=melanoma; 30=uveal melanoma; 31=LGG; 32=GBM.

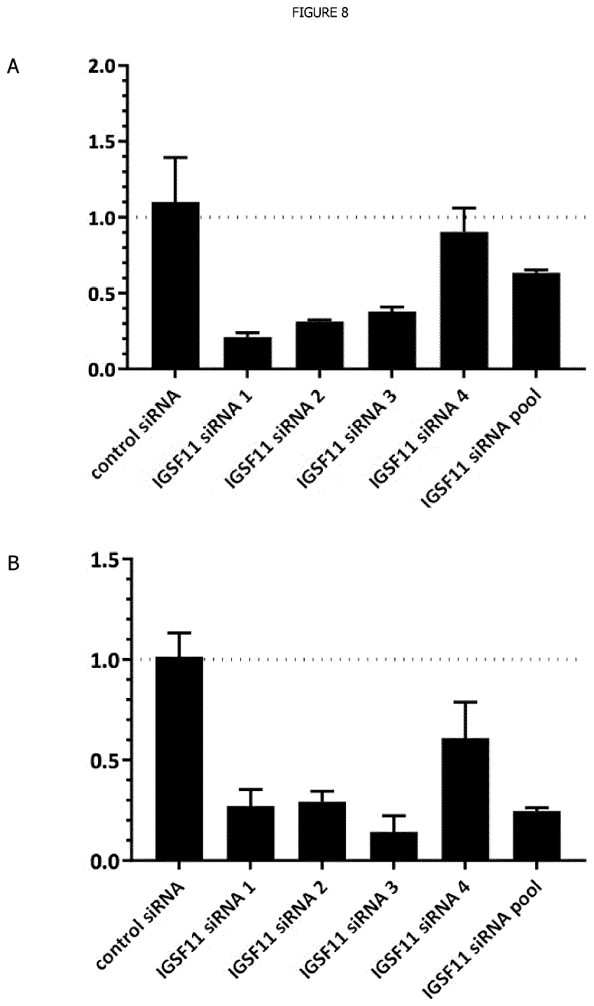

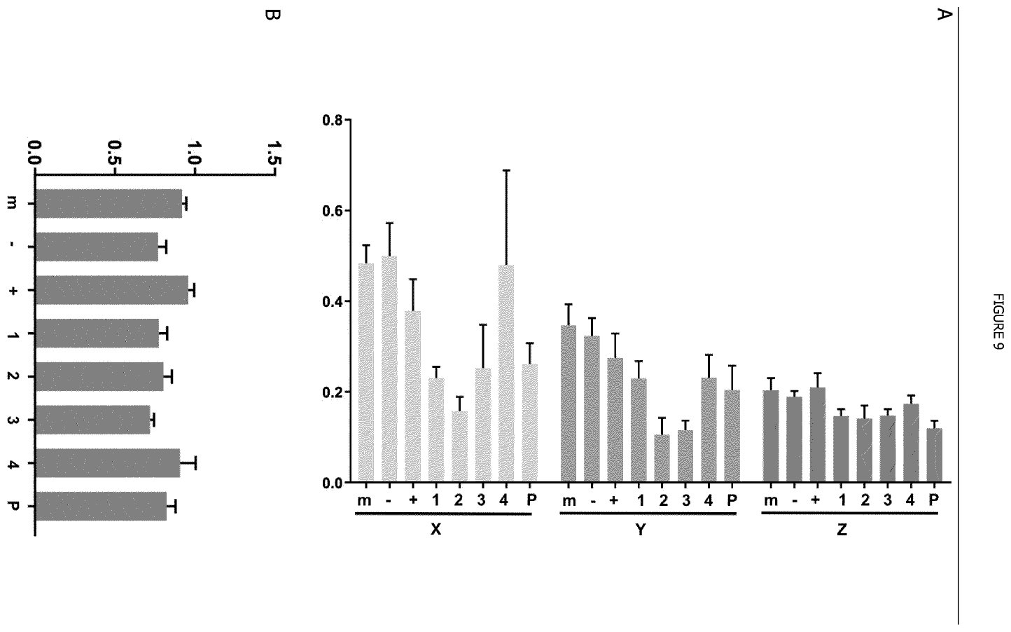

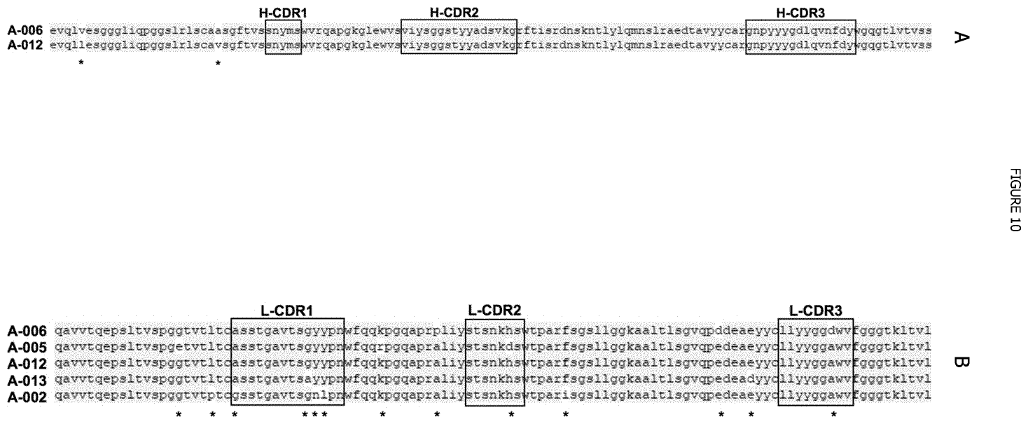

FIG. 8: Knockdown of IGSF11 expression (mRNA level/qPCR) by the siRNA pool and individual siRNAs deconvoluted from the pool for: (A) the melanoma cell line M579; and (B) the lung cancer cell line A549. X-axis: relative expression (Delta-Delta-Ct from qPCR, normalised to scrambled control siRNA).

FIG. 9: (A) Enhanced cytotoxicity of the melanoma cells M579-A2-luc to T cells (“X”: TIL209; “Y”: TIL 412; and “Z”: flu-specific) upon IGSF11 knockdown by siRNA. (B) Limited increase in cytotoxicity of the low-IGSF11-expressing lung cancer cell line A459-luc against flu-specific T cells. X-axes: ratio of cytotoxicity:viability (no T cells); “m”: mock transfection; “−”: negative scrambled control; “+”: positive PD-L1 siRNA control; “1”: IGSF11 siRNA1; “2”: IGSF11 siRNA2; “3”: IGSF11 siRNA3; “4”: IGSF11 siRNA14; and “P”: IGSF11 siRNA pool. Results are cumulative of 3 independent experiments performed in triplicates.

FIG. 10: Alignments of amino acid sequences of variable domains from antibodies of the invention: (A) VH domains of antibodies A-012 and A-013; (B) VL domains of antibodies A-002, A-005, A-006, A-012 and A-013. Locations of the corresponding CDRs are marked, and particular locations of sequence divergence are indicated by “*”.

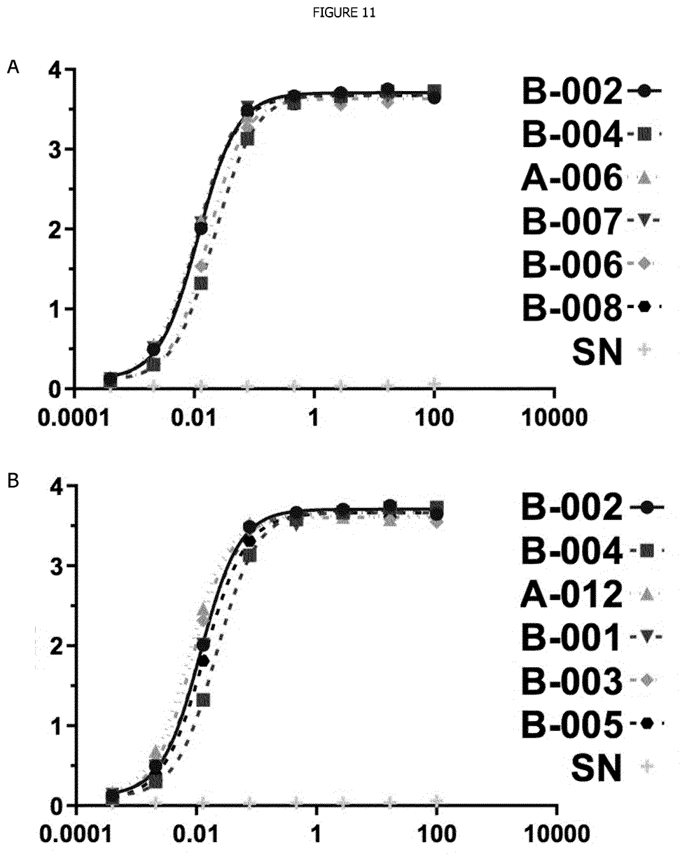

FIG. 11: Binding of antibody and chain-swapped antibodies to His-IGSF11: (A) binding of antibody A-006 and other antibodies comprising either a heavy-chain or light-chain variable region from A-006; and (B) binding of antibody A-012 and other antibodies comprising either a heavy-chain or light-chain variable region from A-012. X-axis: concentration of IgG antibody (nM); and Y-axis: absorbance. “SN”: supernatant control.

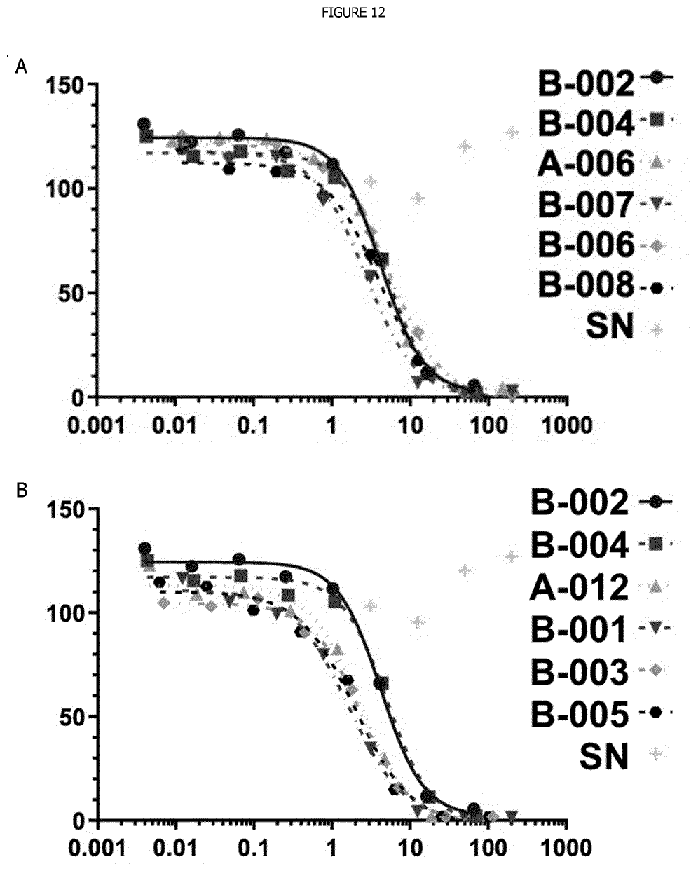

FIG. 12: Inhibition by antibody and chain-swapped antibodies of binding IGSF11 to VSIR: (A) inhibition by antibody A-006 and other antibodies comprising either a heavy-chain or light-chain variable region from A-006; and (B) inhibition by antibody A-012 and other antibodies comprising either a heavy-chain or light-chain variable region from A-012. X-axis: concentration of IgG antibody (nM); and Y-axis: % remaining IGSF11 binding to VSIR. “SN”: supernatant control.

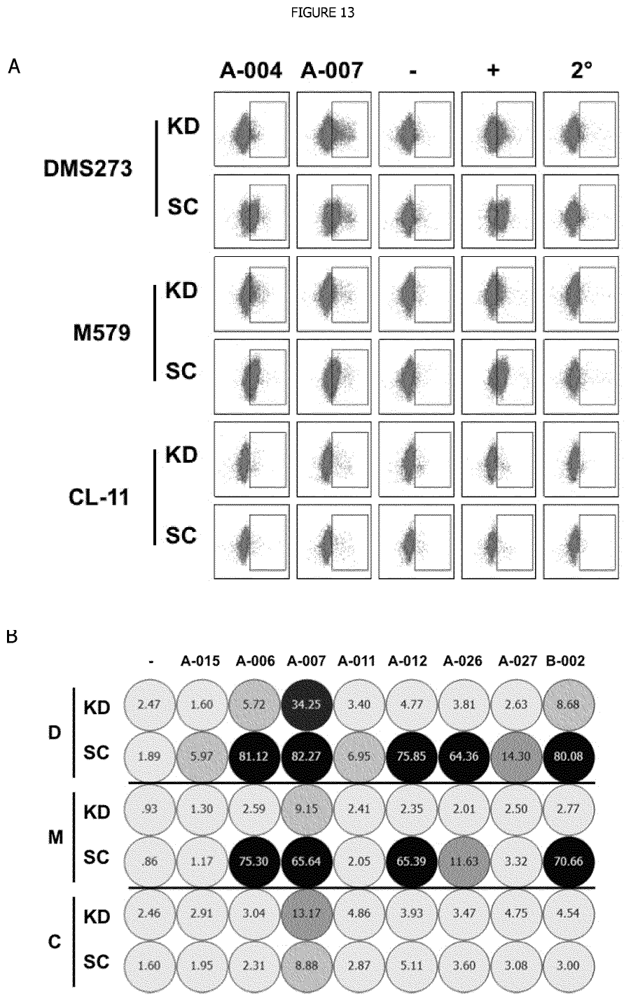

FIG. 13: FACS-detection of binding of antibodies of the invention to the cancer cells lines DMS 273 (“D”), M579-A2-luc (“M”) and CL-11 (C”), treated with either IGSF11 siRNA (“KD”) or scrambled control (“SC”), and binding detected using IgG1-format antibodies of the invention, as well as human isotype negative control (“−”), positive control (“+”) and only secondary antibody (“2°”). (A) Example FACS output as dot-plot; (B) analogous experiment with percentage binding represented as heat-map.

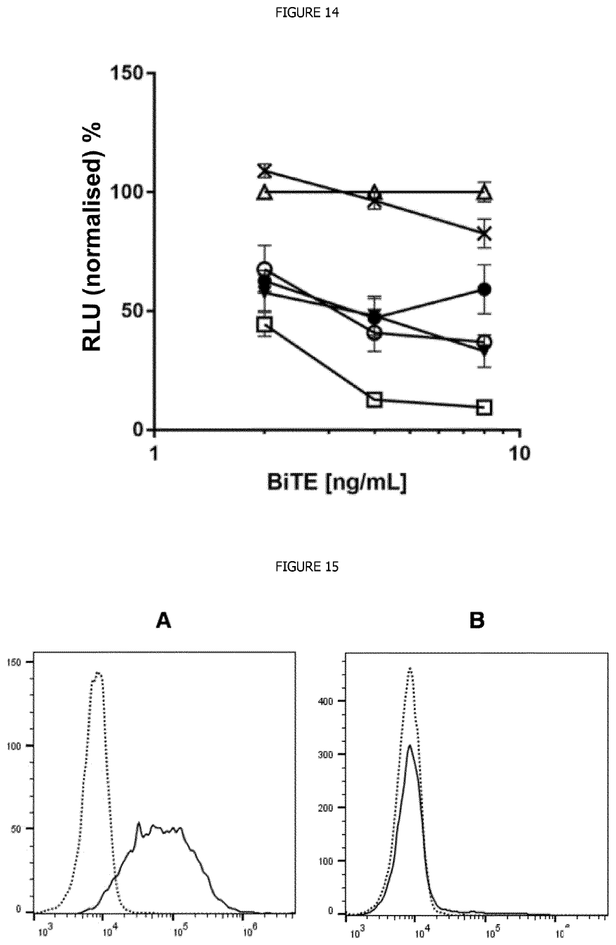

FIG. 14: Antibodies of the invention enhance T cell cytotoxicity against cancer cells. Crosses: BiTE only; Open Triangles: BiTE+isotype control; Open Circles: BiTE+anti-PD-Li antibody; Open Squares: BiTE+anti-VISTA antibody; Solid Triangles: BiTE+A-006 antibody of the invention; Solid Circles: BiTE+A-012 antibody of the invention. X-axis: concentration of BiTE molecule in assay; Y-Axis tumour cell viability represented as relative luciferase values (RLU), normalised to “BiTE+isotype control” (%).

FIG. 15: MDA-MB-231-luc cells were transduced with IGSF11-encoding lentiviral vector (A) based on p443MYCIN (proQinase) or mock transduced (B). IGSF11 overexpression on cell surface was confirmed using flow cytometry staining with A-006 antibody followed by anti-human IgG-AF647 secondary Ab. X-axis: IGSF11 signal; Y-axes: number of events (cells).

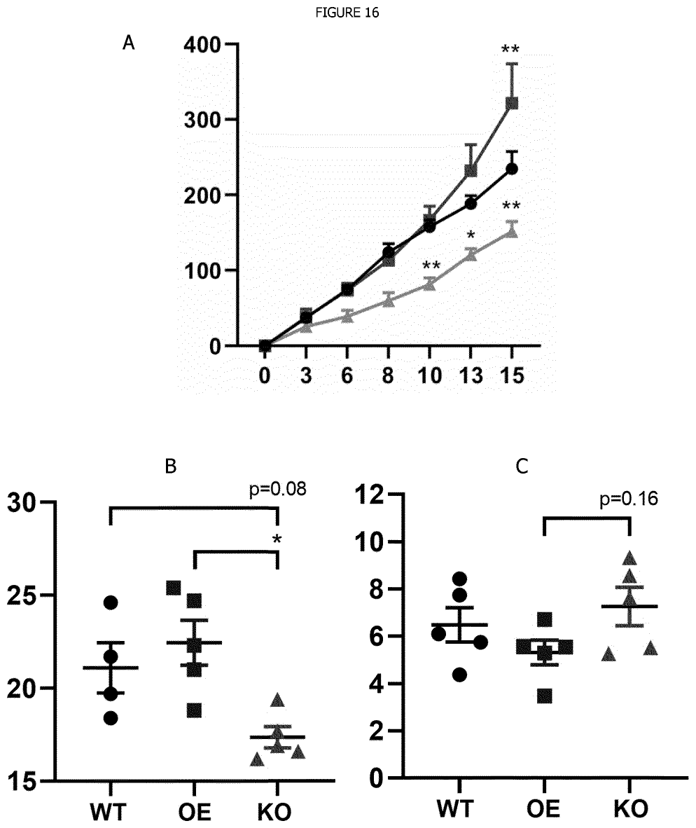

FIG. 16: In-vivo relevance of IGSF11 in a syngeneic mouse model (A). Tumour growth curves of the mock-transduced Wild-type (“WT”), IGSF11-knockout (“KO”) and IGSF11-overexpressing (“OE”) MC38 murine tumour cells. KO tumours (triangles) are rejected better than WT tumours (circles) by the immune systems and IGSF11 OE tumours (squares) show a stronger growth as they suppress the immune system of the mouse. X-axis: Days after inoculation; Y-axis: Tumour volume (mm3). “**”=P<0.01 compared to WT mock transduction control, “*”=P<0.05 compared to WT mock transduction control. Decrease in intra-tumoral gMDSCs (B), and increase in intra-tumoral CTLs (C) in KO tumours compared to OE tumours and/or WT (mock control). Y-axes: % viable intra-tumoural cells.

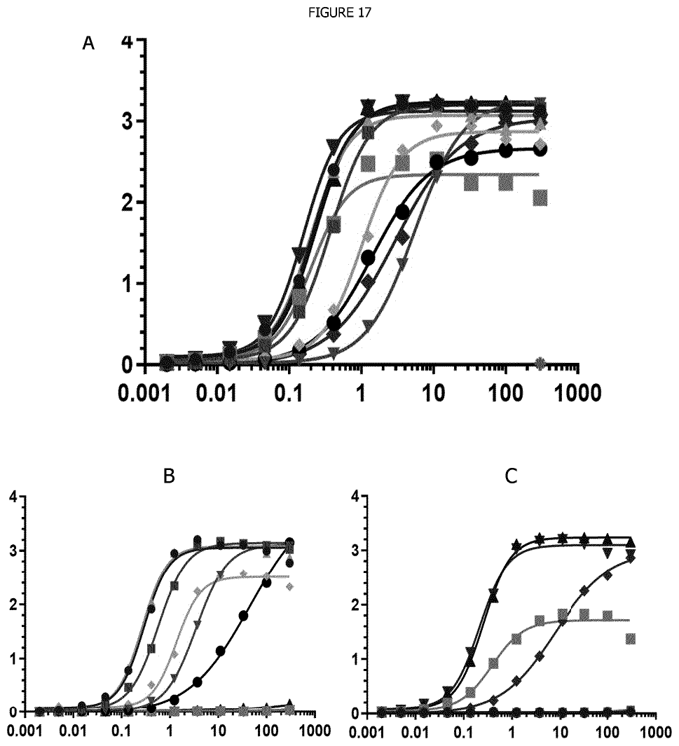

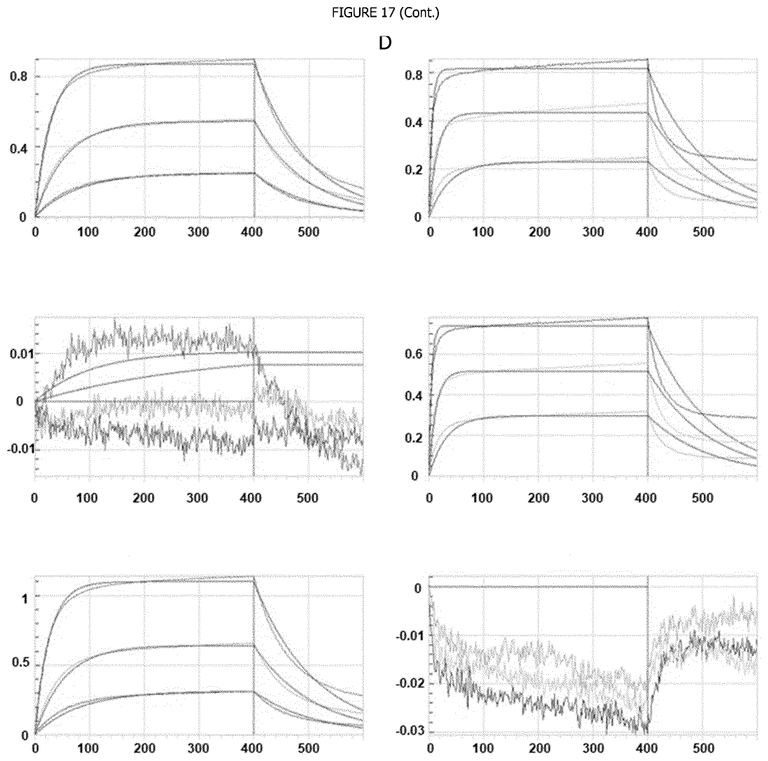

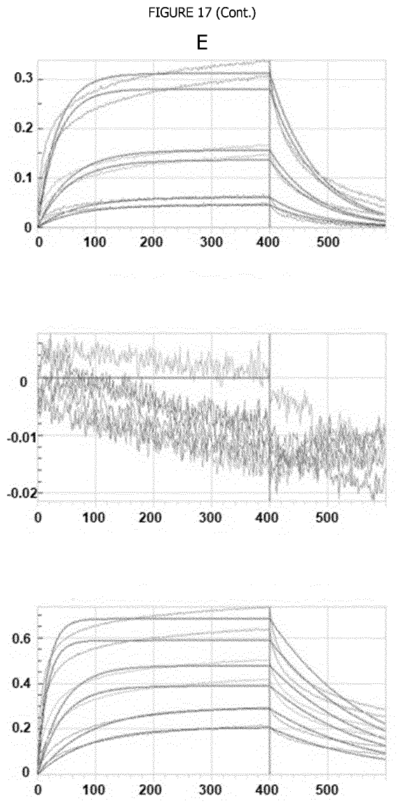

FIG. 17: Binding of IgG antibodies of the Comparative Examples to the full-length ECD of IGSF11 (A). Binding of IgG antibodies of the Comparative Examples to the IgC2 domain of IGSF11 (B). Binding of IgG antibodies of the Comparative Examples to the IgV domain of IGSF11 (C). X-axes: IgG concentration (nM); Y-axes: Absorption (arbitrary units). BLI experiments testing binding of A-006-like (left column) or A-024-like (right column) IGSF-binding ABPs (in IgG1 format) to the entire ECD of IGSF11 (top row), the IgV domain of IGSF11 (middle row) and the IgC2 domain of IGSF11 (bottom row) (D). BLI experiments testing binding of a VSIR protein (in multimer format) to the entire ECD of IGSF11 (top row), the IgV domain of IGSF11 (middle row) and the IgC2 domain of IGSF11 (bottom row) (E). X-axes: Time (s); Y-axes: Response (nm)

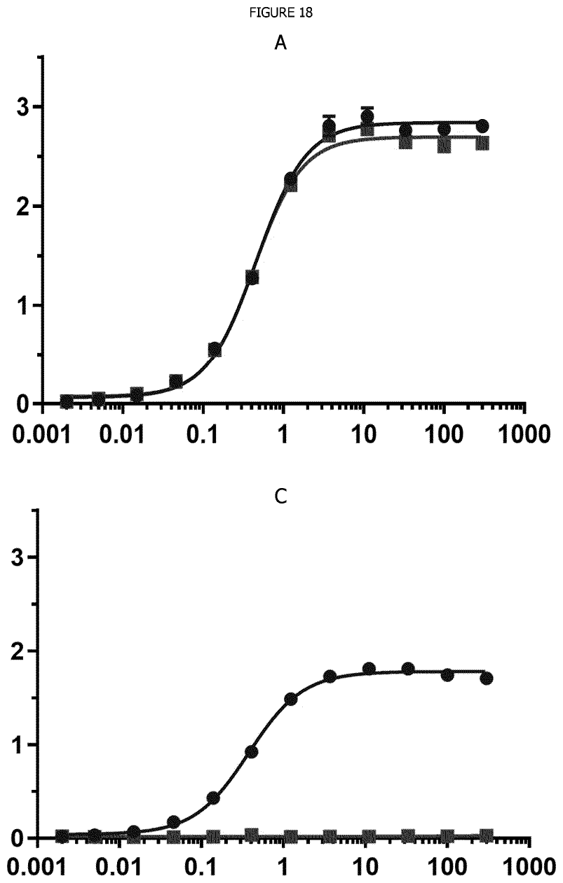

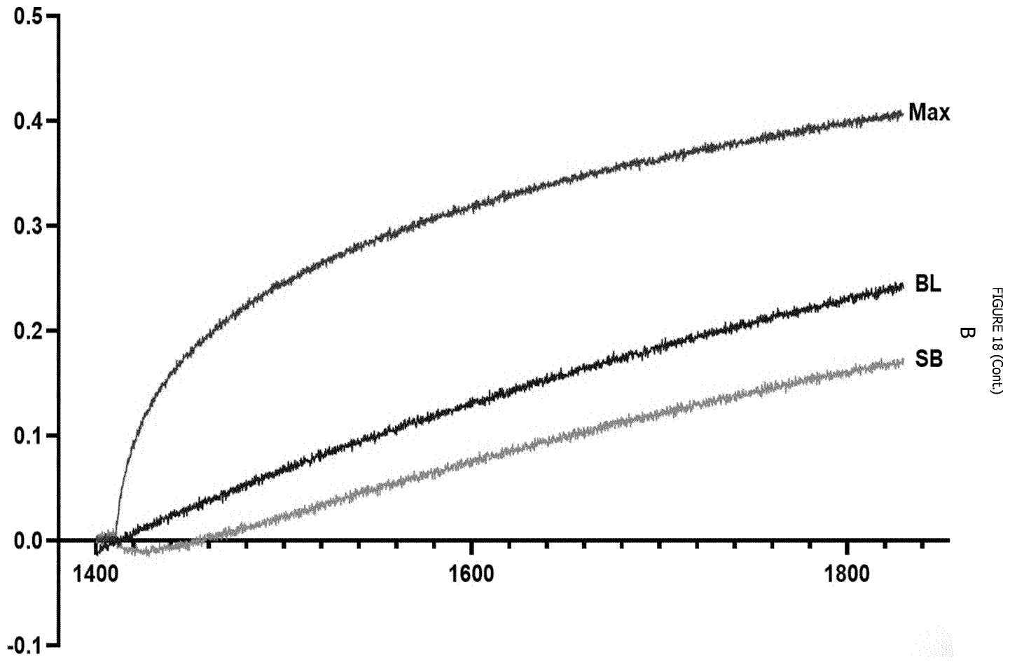

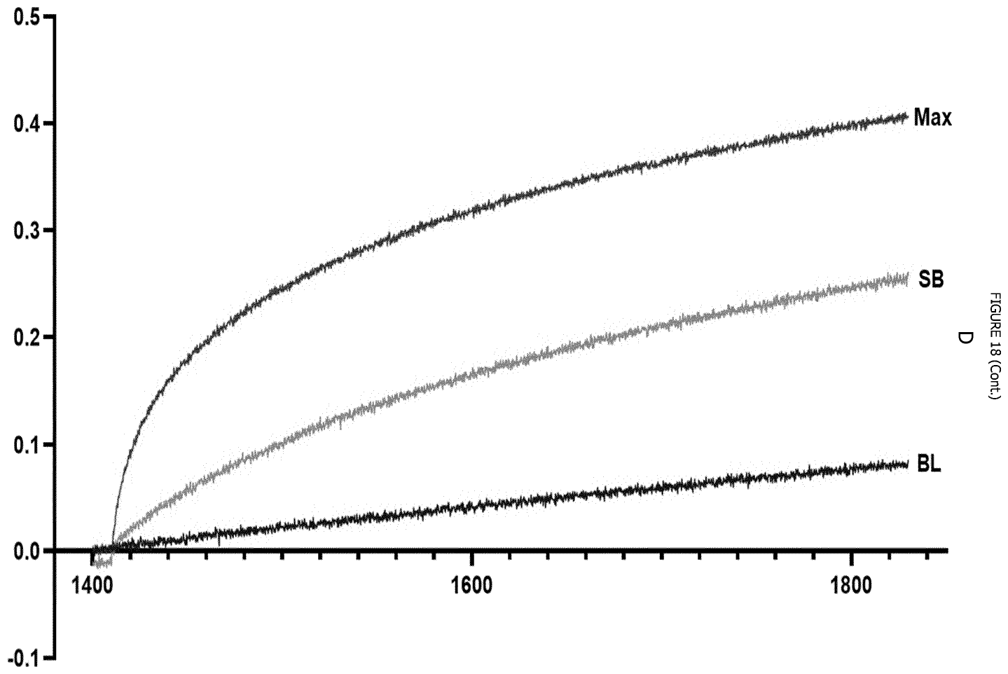

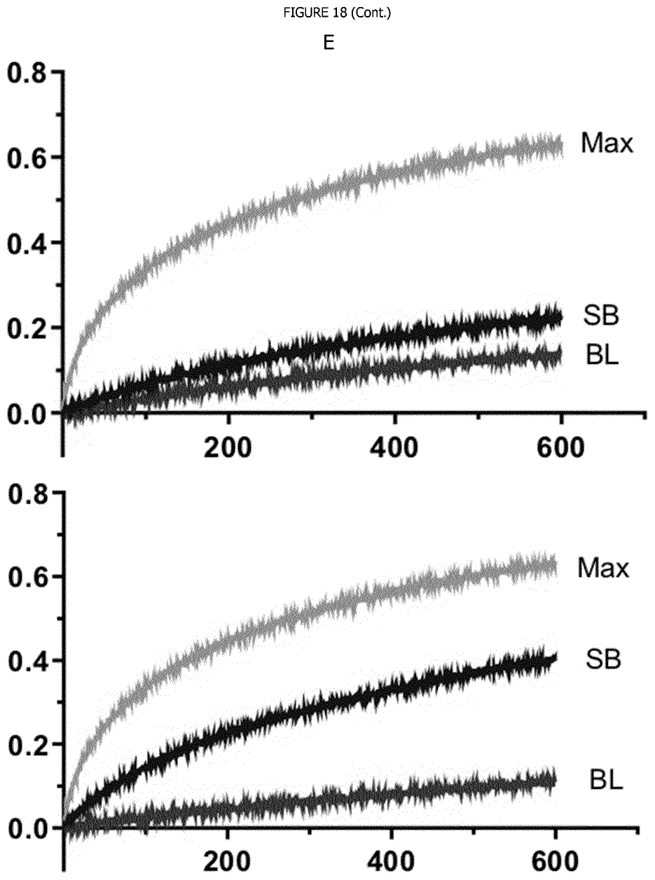

FIG. 18: Binding of A-006-like ABPs to full-length ECD (circles) of IGSF11 or to the IgC2 domain (squares) of IGSF11 (A), and BLI curves showing their competition with VSIR for binding to surface-bound IGSF11 (B). Binding of A-024-like ABPs to full-length ECD (circles) of IGSF11 or to the IgC2 domain (squares) of IGSF11 (C), and BLI curves showing no competition with VSIR for binding to surface-bound IGSF11 (D). BLI curves showing competition of IgC2 domain binding ABP C-004 with VSIR for binding to surface-bound IGSF11 (upper), and showing no competition of IgV domain binding ABP C-001 with VSIR for binding to surface-bound IGSF11 (E). (A) and (C), X-axes: IgG concentration (nM); Y-axes: Absorption (arbitrary units). (B), (D) and (E), X-axes: Time (s); Y-axes: Response (nm); “BL”=baseline; “Max”=maximum VSIR binding; “SB”=VSIR simultaneous binding.

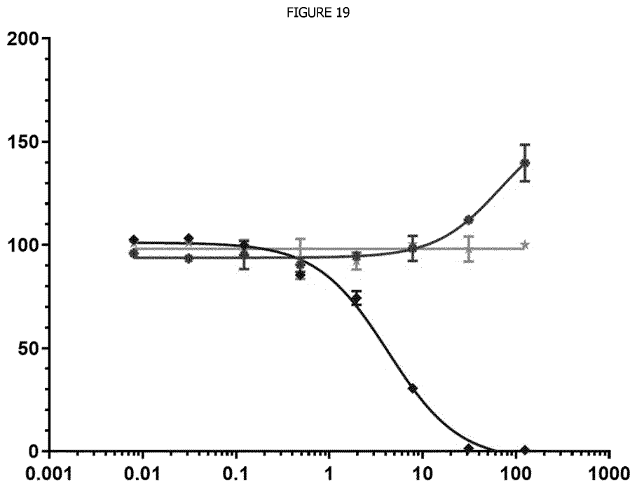

FIG. 19: Inhibition of binding of IGSF11 to bound VSIR by IgC2 domain-binding A-006-like ABPs (diamonds) compared to IgV domain-binding A-024-like ABPs (circles) and to “Ref001”, an isotype control ABP (stars). X-axis: IgG concentration (nM); Y-axis: % remaining IGSF11 binding.

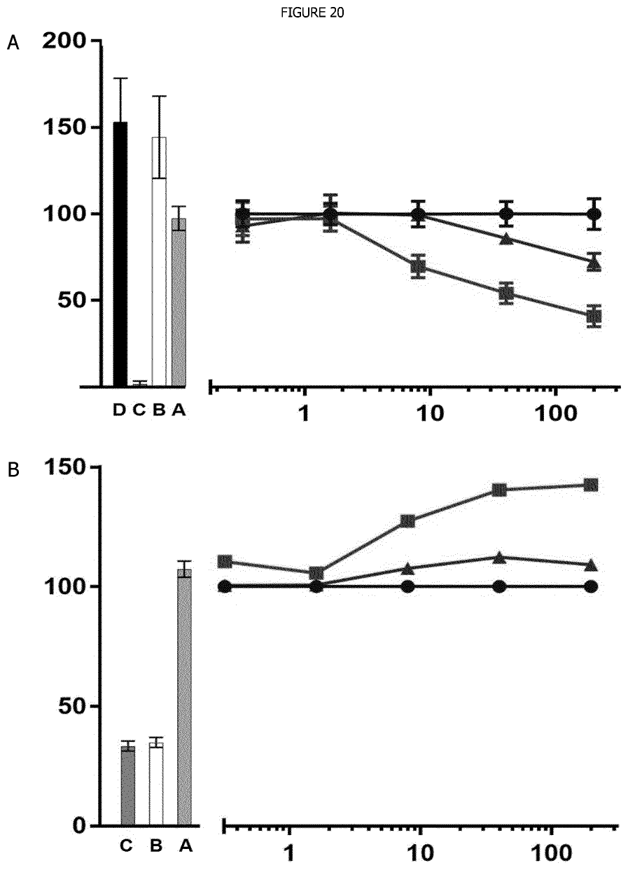

FIG. 20: Enhanced T cell-mediated killing of IGSF11-expressing MBA-MB-231 cells by an IgC2 domain-binding A-006-like ABP in the presence of an anti-EpCamxCD3 “BiTE” (squares), compared to an IgV domain-binding A-024-like ABP (triangles) and to “Ref001”, an isotype control ABP (circles) (A). X-axis: Antibody concentration (ug/mL), Y-axis: Tumour lysis (%)—normalised. Such tumour cell killing correlates with T cell activation (B). X-axis: Antibody concentration (ug/mL), Y-axis: Mean fluorescent intensity of CD69-staining. A=tumour cells+BiTE+T cells; B=tumour cells+ T cells; C=T cells only; D=tumour cells only.

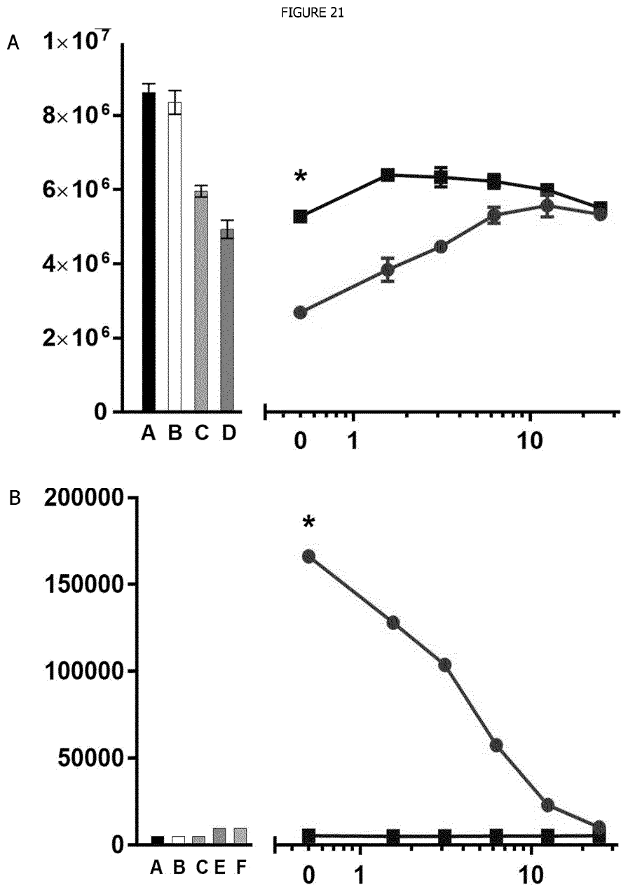

FIG. 21: Soluble IGSF11 abolishes T cell-mediated killing of IGSF11-expressing MBA-MB-231 cells by an IgC2 domain-binding A-006-like ABP in the presence of an anti-EpCamxCD3 “BiTE” (circles), compared to “Ref001” an isotype control ABP (squares) (A). X-axis: IGSF11-His protein (ug/mL), Y-axis: RLU (relative luminescence units). Soluble IGSF11 inhibits tumour cell-binding of a_A-006-like ABP (circles), compared to “Ref001” an isotype control ABP (squares) (B). X-axis: IGSF11-His protein (ug/mL), Y-axis: IGSF11 binding RLU (relative luminescence units); A=tumour cells only; B=tumour cells+ T cells; C=tumour cells+BiTE+ T cells; D=tumour cells+BiTE+IGSF11+ T cell; E=tumour cells+BiTE+IGSF11 (high)+A-006-like ABP; F=tumour cells+IGSF11 (high)+T cell+A-006-like ABP; *=condition without protein (tumour cells+BiTE+ T cells+ABP).

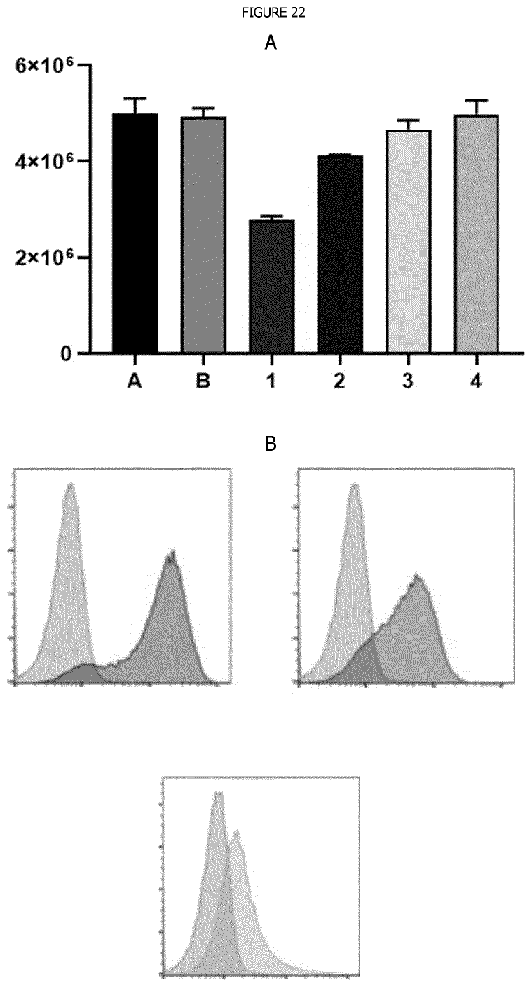

FIG. 22: An IgC2 domain-binding A-006-like ABP (1) shows enhanced T cell-mediated cell killing of COLO-741 cells that naturally express IGSF11, compared to an IgV domain binding A-024-like ABP (2), “Ref001” an isotype control ABP (3) and anti-PDL1 antibody (4) (A). Y-axis: RLU (relative luminescence units); A=tumour cells only; B=tumour cells+ T cells. FACS staining of COLO-741 cells for IGSF11 expression using the IgC2 domain binding ABP (top left) and the IgV domain binding ABP (top right); for PDL1 expression (bottom) (B).

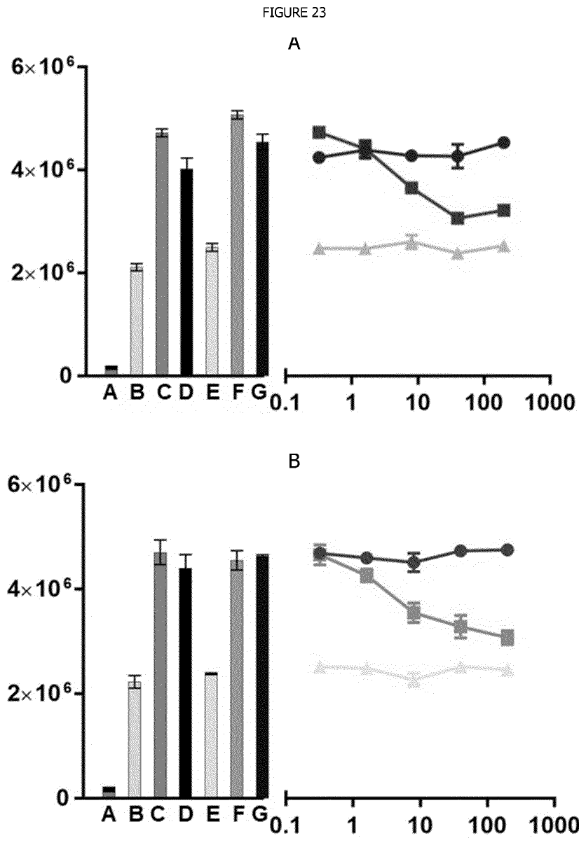

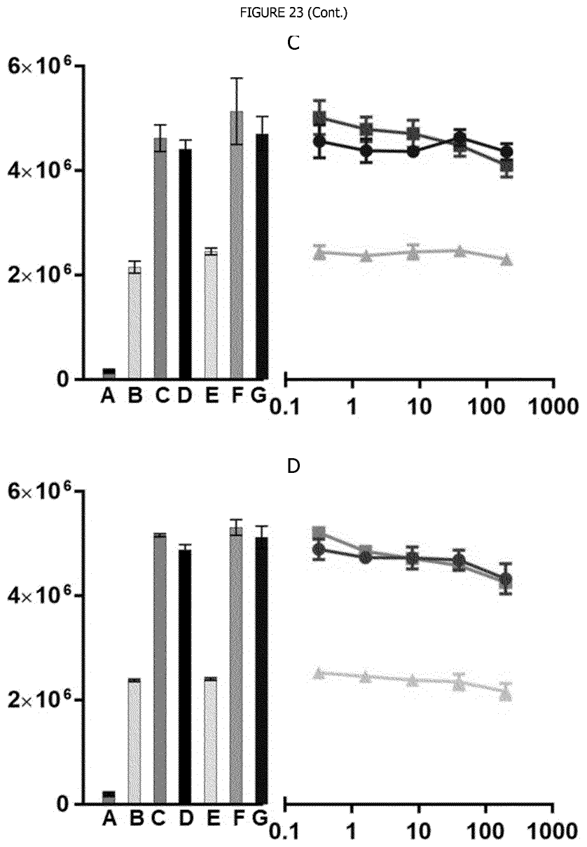

FIG. 23: Cell killing of COLO-741 cells by two IgC2 domain-binding A-006-like ABPs is dependent on the presence of T cells (squares) compared to no T cells (circles), and not merely T cell supernatant (triangles) (A) and (B). No T cell-mediated killing is observed for two IgV domain-binding ABPs (C) and (D). X-axes: Antibody concentration (ug/mL). Y-axes: RLU (relative luminescence units); A=T cells only; B=tumour cells+ T cells suspension; C=tumour cells+ T cells; D=tumour cells only; E=tumour cells+“Ref001” (isotype control ABP, 200 ug/ml)+ T cells suspension; F=tumour cells+“Ref001” (200 ug/ml)+ T cells; G=tumour cells+“Ref001” (200 ug/ml).

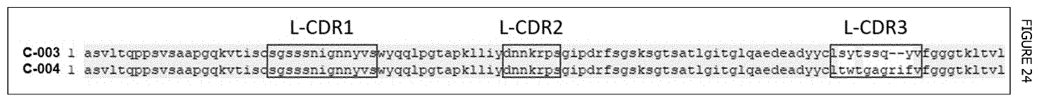

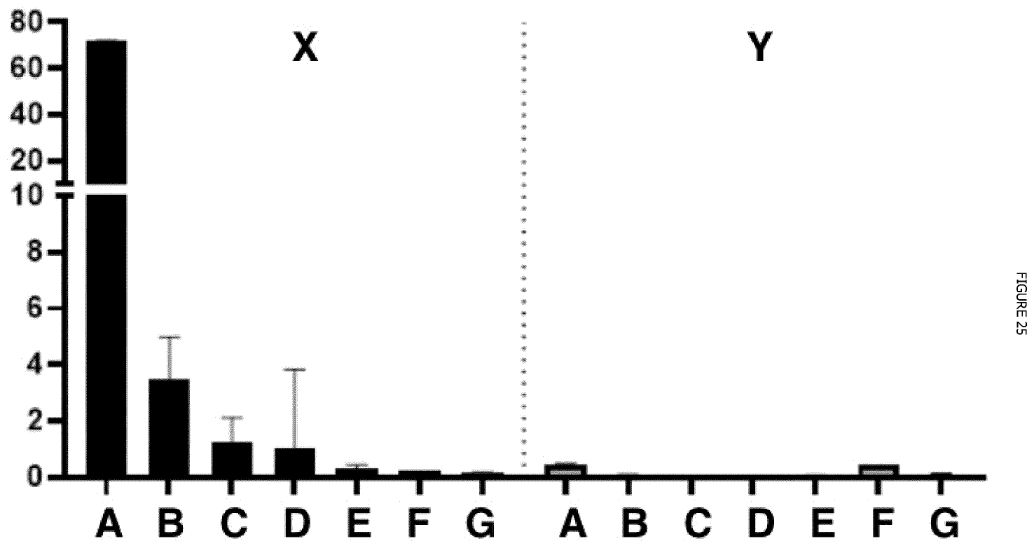

FIG. 24: Amino acid sequence alignment of the light-chains of ABPs C-003 and C-004, showing the position of the L-CDR1, L-CDR2 and L-CDR3.

FIG. 25: Expression of VISTA (X) and IGSF (Y) on various immune cells. Y-axis=Percentage of positive cells (normalised to isotype control). Immune cell-types: A=CD14+; B=CD56+; C=CD3+; D=CD19+; E=M1 macrophages; F=M2 macrophages; and G=M0 macrophages.

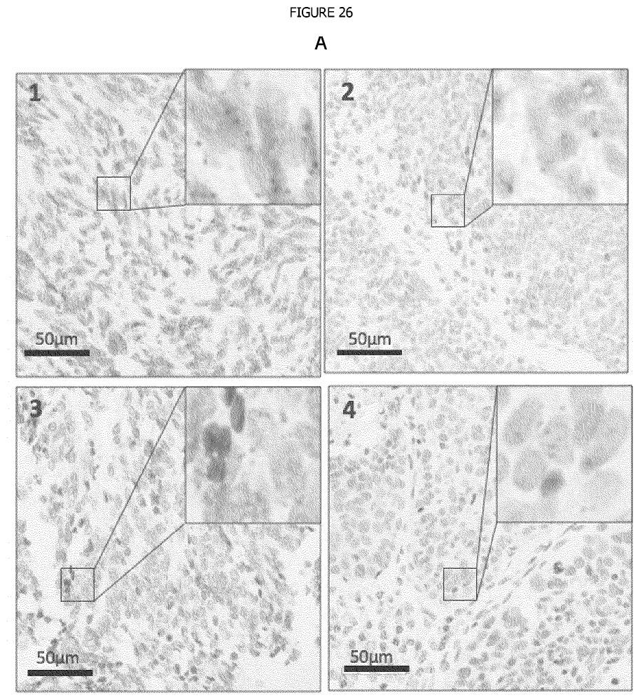

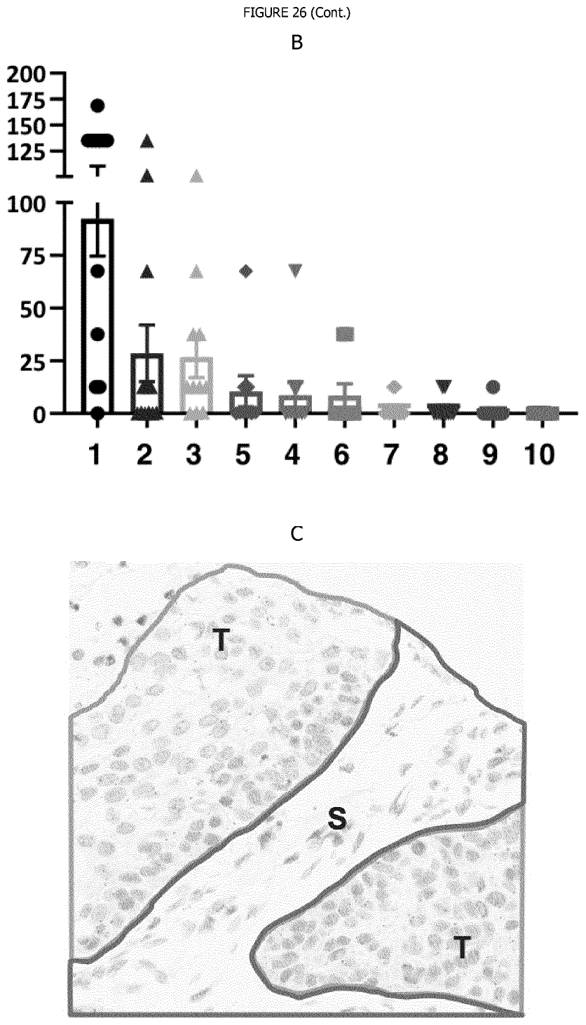

FIG. 26: IGSF11 is exclusively expressed on tumour cells in multiple solid tumours (A) and (B), but not on infiltrating stroma (C). Tumour types: 1=melanoma; 2=head and neck squamous cell carcinoma (HNSCC); 3=ovarian cancer; 4=squamous lung cancer; 5=pancreas; 6=bladder; 7=prostate; 8=colorectal; 9=breast and 10=kidney. (B) Y-axis; IGSF11 expression rate (scope×% positive cells). (C) T=tumour tissue in an HNSCC case; S=stroma.

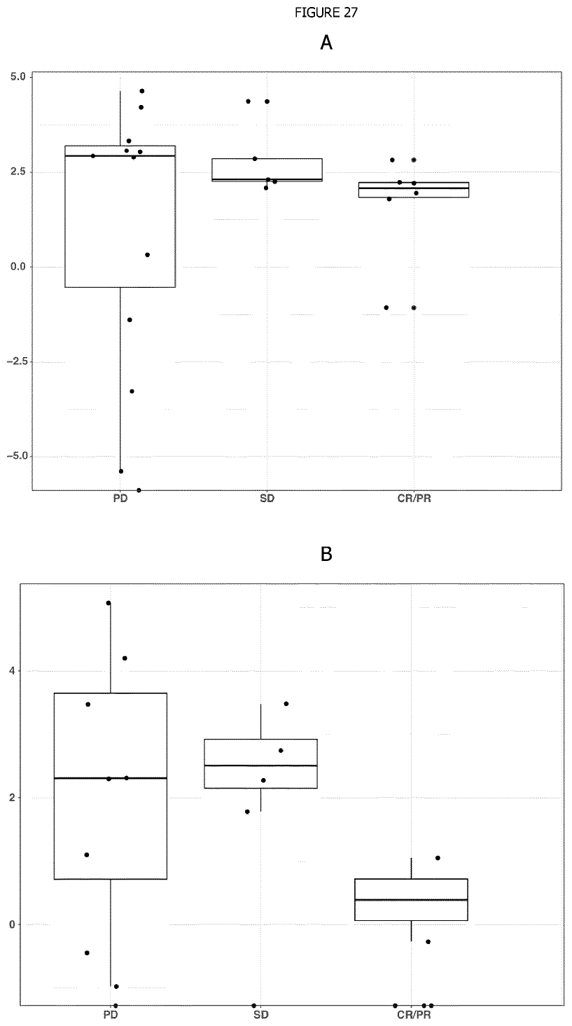

FIG. 27: Expression of IGSF11 in 33 responding and non-responding melanoma patients treated with nivolumab (Riaz et al 2017), before treatment (A) and upon treatment (B). Y-axes=IGSF11 expression [log 2(TPM)]; PD=progressive disease; CR/PR=complete or partial response; SD=stable disease. C) Negative correlation of IGSF11 with a multi-gene measure of tumour inflammation (X-axis).

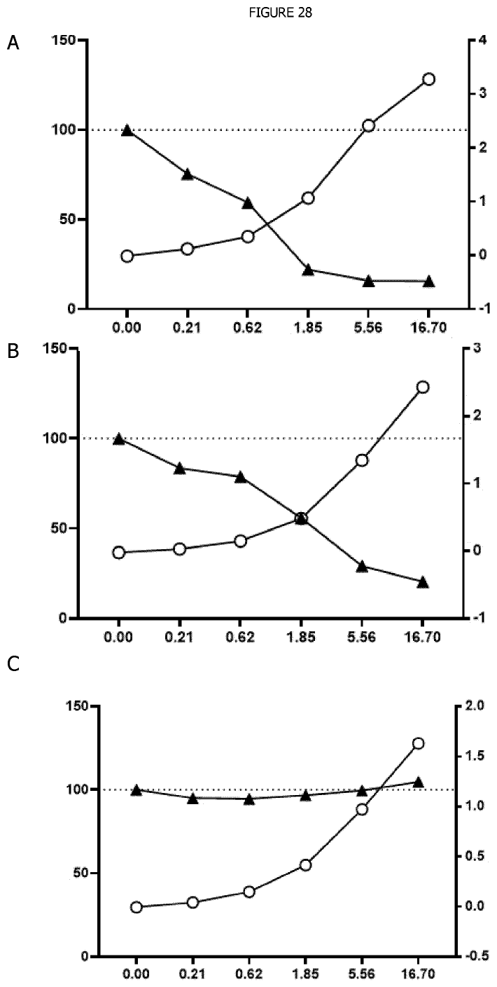

FIG. 28: depicts inhibition of IGSF11-VISTA binding by ABPs that bind the IgC2 domain of IGSF11: D-114 (A); and D-222 (B) compared to an ABP that binds to the IgV domain of IGSF11: C-001 (C). Binding of the APBs to IGSF11 (open circle; right hand Y axes; IgG Association Response (nm)) was plotted against remaining VISTA binding (solid triangle; left hand Y axes; VISTA-comp binding (%); binding response of simultaneously binding VISTA was normalized to binding response of VISTA without prior antibody binding). X axes=APB concentration (nM).

The present invention, and particular non-limiting aspects and/or embodiments thereof, can be described in more detail as follows:

In one aspect, and as may be further described, defined, claimed or otherwise disclosed herein, the invention relates to a method for (or of) identifying and/or characterising an ABP as one specifically binding to a C2-type immunoglobulin-like (IgC2) domain of IGSF11 (VSIG3) protein or a variant thereof, the method comprising the step of: (X) detecting binding of the ABP to an epitope of (or comprised in) such domain of IGSF11 protein (or variant thereof), thereby identifying and/or characterising the ABP as one that specifically binds to the IgC2 domain of IGSF11 protein or variant thereof.

In one alternative other aspect, and as may be further described, defined, claimed or otherwise disclosed herein, the invention relates to a method for (or of) identifying and/or characterising an ABP as one specifically binding to a V-type immunoglobulin-like (IgV) domain of IGSF11 (VSIG3) protein or a variant thereof, in one embodiment, the method comprising the step of: (X) detecting binding of the ABP to an epitope of (or comprised in) such domain of IGSF11 protein (or variant thereof), thereby identifying and/or characterising the ABP as one that specifically binds to the IgV domain of IGSF11 protein or a variant thereof.

In one embodiment of such aspect, the method, further comprises the step of: (Y) testing for binding of the ABP to an epitope of (or comprised in) an IgV domain of IGSF11 protein or a variant thereof (or, in the other aspect, to an epitope of, or comprised in, an IgC2 domain of IGSF11 protein or a variant thereof), wherein, absence of detectable (or, of substantial or appreciable) binding of the ABP to the epitope of (or comprised in) such domain of IGSF11 protein (or variant thereof) further characterises the ABP as one that specifically binds to the IgC2 domain of IGSF11 protein or variant thereof (or, in the other aspect as one that specifically binds to the IgV domain of IGSF11 protein or variant thereof).

Testing for binding to IGSF11 protein, to a domain of IGSF11 protein or to an epitope of (or comprised in) a domain of IGSF11 protein, such as an IgC2 domain of IGSF11 protein (or an IgV domain of IGSF11 protein), or a variant thereof, may be conducted by any suitable methodology as will be known by the person of ordinary skill. For example, binding, or an interaction between, a (eg, test) ABP and a given antigen (eg IGSF11 protein, a domain thereof or an epitope of or comprised in such protein or domain) can be tested by techniques such as ELISA, biolayer interferometry or surface plasmon resonance. The Examples, and/or the Comparative Examples, herein provide summary details of techniques and methods that may be used to conduct such testing for binding, or an interaction between, an ABP and the antigen.

Further embodiments of such methods include those, wherein: the detecting step (X) comprises detecting binding of the ABP to a first test protein, wherein the first test protein: (i) comprises the IgC2 domain of IGSF11 or a variant or fragment of such domain; and (ii) does not comprise an IgV domain of IGSF11 or, optionally, a variant of such domain (or, in the other aspect: (i) comprises the IgV domain of IGSF11 or a variant or fragment of such domain; and (ii) does not comprise an IgC2 domain of IGSF11 or, optionally, a variant of such domain); and/or the testing step (Y) comprises testing for binding of the ABP to a second test protein, wherein the second test protein: (a) comprises the IgV domain of IGSF11 or a variant or fragment of such domain thereof; and (b) does not comprise the IgC2 domain of IGSF11, or a variant or fragment of such domain (or, in the other aspect, (a) comprises the IgC2 domain of IGSF11 or a variant or fragment of such domain thereof; and (b) does not comprise the IgV domain of IGSF11, or a variant or fragment of such domain).

In the context of such methods, the test proteins will, typically, be proteins that have been engineered (eg, by genetic recombination) to contain one or the other of an IgC2 or the IgV domain of IGSF11 protein (or variants or fragments of such domain). Elsewhere herein (eg in the Examples) are described IgC2 and IgV domains, and how they may be prepared/produced and used on such binding assays.

For example, in certain embodiments of the methods: the first test protein does not comprise an IgV domain of IGSF11 (or, in the other aspect, does not comprise an IgC2 domain of IGSF11) or a variant or fragment of such domain; and/or the second test protein comprises the IgV domain of IGSF11 (or, in the other aspect, comprises the IgC2 domain of IGSF11) or variant thereof.

The ABP and the optional first test protein can be, in particular embodiments, provided prior to the detecting step and/or the ABP and the optional second test protein can be, in particular embodiments, provided prior to the testing step.

In such methods, it can be of particular utility that an ABP identified and/or characterised as one that specifically binds to the IgC2 domain of IGSF11 protein (or, in the other aspect, as one that specifically binds to the IgV domain of IGSF11) or variant thereof is further identified and/or characterised as one for use in medicine.

Accordingly, in another aspect, and as may be further described, defined, claimed or otherwise disclosed herein, the invention relates to a method for (or of) identifying and/or characterising an ABP for use in medicine, said method comprising the steps of: providing an ABP that binds to IGSF11 protein; and identifying and/or characterising the provided ABP as one that specifically binds to an IgC2 domain of IGSF11 protein (or, in another aspect, as one that specifically binds to an IgV domain of IGSF11 protein) or a variant thereof, thereby identifying and/or characterising the ABP for use in medicine.

In a related aspect, and as may be further described, defined, claimed or otherwise disclosed herein, the invention relates to a method for (or of) producing an ABP for use in medicine, the method comprising the steps of:
providing a hybridoma or (host) cell capable of expressing an ABP that binds to IGSF11 protein, for example a recombinant cell line comprising at least one genetic construct comprising coding sequence(s) encoding said ABP; and
culturing said hybridoma or host cell under conditions that allow for the expression of the ABP;
optionally, isolating the ABP expressed by said hybridoma or host cell; and
identifying and/or characterising the expressed ABP as one that specifically binds to an IgC2 domain of IGSF11 protein (or, in another aspect, as one that specifically binds to an IgV domain of IGSF11 protein) or a variant thereor,
   thereby producing the ABP for use in medicine.

In particular embodiments of these another and related aspects, the ABP is identified and/or characterised as specifically binding to such domain of IGSF11 protein (or variant) by a method of the above aspects.

In a further related aspect, and as may be further described, defined, claimed or otherwise disclosed herein, the invention relates to a use of an IgC2 domain of IGSF11 protein (or, in another aspect, of an IgV domain of IGSF protein) or a variant or fragment of such domain (eg, at least one epitope of or comprised in such domain) to identify, characterise and/or produce an ABP, eg for use in medicine, suitably wherein the ABP specifically binds to such domain of IGSF11 protein or variant thereof.

In such use, certain embodiments may further comprise the use of an IgV domain of IGSF11 protein (or, in the other aspect, the use of an IgC2 domain of IGSF11 protein) or, optionally, a variant thereof, suitably wherein the ABP does not bind to such domain of IGSF11 protein (or variant).

In particular embodiments, such use may further comprise the use of:
a first test protein, wherein the test protein: (i) comprises the IgC2 domain of IGSF11 or a variant or fragment of such domain; and (ii) does not comprise an IgV domain of IGSF11 (or, in the other aspect, (i) comprises the IgV domain of IGSF11 or a variant or fragment of such domain; and (ii) does not comprise an IgC2 domain of IGSF11); and/or
a second test protein, wherein the second test protein: (a) comprises an IgV domain of IGSF11 or a variant or fragment of such domain thereof; and (b) does not comprise the IgC2 domain of IGSF11, or a variant or fragment of such domain (or, in the other aspect, (a) comprises an IgC2 domain of IGSF11 or a variant or fragment of such domain thereof; and (b) does not comprise the IgC2 domain of IGSF11, or a variant or fragment of such domain).

For example, in such use:
the first test protein may not comprise an IgV domain of IGSF11 or a variant or fragment of such domain (or, in the other aspect, may not comprise an IgC2 domain of IGSF11 or variant or a fragment of such domain); and/or
the second test protein may comprise the IgV domain of IGSF11 (or, in the other aspect, may comprise the IgC2 domain of IGSF11) or, optionally, a variant thereof.

In particular embodiment of a such use of an IgC2 (or IgV) domain of IGSF11 (or variant thereof) to identify, characterise and/or produce an ABP, comprises the screening of a phage or other library that displays a plurality of candidate ABPs (eg a phage antibody library), and an ABP that is found to bind to such domain is thereby identified. Another such use comprises the immunisation an animal, in particular a mammal (such as a mouse, rat, rabbit, goat, camel, alpaca or llama) with such domain as a protein, or as a nucleic acid that encodes said domain, and the isolation of sera that contains, or B cells that express, an ABP that binds to such domain.

Accordingly, in a particular aspect, the invention also relates to a method for (or of) identifying, generating and/or producing an ABP that (eg, specifically) binds to an IgC2 domain of IGSF11 (or to an IgV domain of IGSF11), or a variant or fragment/epitope thereof, the method comprising the use of such domain (or variant or fragment/epitope): (i) to screen a display library of a plurality of ABPs (eg a phage display library); or (ii) to immunise an animal, in particular a mammal (such as a mouse, rat, rabbit, goat, camel or llama). Further steps and embodiments of such aspect are described elsewhere herein, in particular the section discussing types of ABPs, their generation and modification.

In preferred of these embodiments, when the IgC2 domain of IGSF11 (or variant or fragment/epitope thereof) is used in the screen or immunisation (either as protein or nucleic acid encoding such domain or at least one or more epitopes thereof or comprised therein), then such protein does not comprise the IgV domain of IGSF11 or does not comprise an epitope thereof (or the nucleic acid does not encode a protein comprising the IgV domain of IGSF11 or epitope thereof), or a variant thereof. Correspondingly, preferred of these embodiments, when the IgV domain of IGSF11 (or variant or fragment/epitope thereof) is used in the screen or immunisation (either as protein or nucleic acid encoding such domain or epitope thereof), then such protein does not comprise the IgC2 domain of IGSF11 or does not comprise an epitope thereof (or the nucleic acid does not encode a protein comprising the IgC2 domain of IGSF11 or epitope thereof), or a variant thereof.

In particular of such embodiments, a display library (eg, a phage display library) is screened that displays a plurality of ABPs, where, preferably, such library is screened for ABPs that bind such protein.

Immunising an animal, in preferred embodiments, comprises a step of administering to the animal an immunisation composition comprising such IgC2 (or IgV) domain of IGSF11 or variant thereof or at least one or more epitopes thereof or comprised therein (eg, either as a protein or as a nucleic acid encoding such domain or epitope thereof), and optionally together with a pharmaceutically acceptable carrier and/or excipient, more preferably such immunisation composition comprises one or more adjuvants. An immunising composition in accordance with the invention elicits an immune response in the immunised animal which is specific for the IgC2 (or IgV) domain of IGSF11 (or variant thereof), preferably by generation of antibodies against such protein. Following immunisation, certain such embodiments of the invention can include a further step of isolating from the animal: (i) sera that comprises an ABP that specifically binds to said domain of IGSF11 (or variant thereof); and/or (ii) B cells that express an ABP that specifically binds to said domain of IGSF11 (or variant thereof).

In any of such method or use aspects, an ABP for use in medicine is, typically (and eg as described in further detail elsewhere herein):
an ABP for use in the treatment of a proliferative disorder that is associated with the undesired presence of IGSF11-positive cells or cells positive for a variant of IGSF11 and/or that is associated with cellular resistance against a cell-mediated immune response and/or that is associated with expression or activity of IGSF11 or a variant thereof of IGSF11, suitable wherein cells involved in the proliferative disorder are resistant to a cell-mediated immune response;
an ABP for use in enhancing an immune response in a mammalian subject, preferably for use in aiding a cell-mediated immune response in a subject such as the subject's T cell mediated immune response, for example for treating a proliferative disease, such as a cancer disease, of for treating an infectious disease; and/or
an ABP for use in the treatment of a proliferative disorder resistant and/or refractory to PD1/PDL1 and/or CTLA4 blockade therapy.

Alternatively, or in addition, in any of such method or use aspects, the ABP (and eg as described in further detail elsewhere herein):
is able to enhance or increase (eg, enhances or increases) killing and/or lysis of cells expressing IGSF11 or an IgC2 domain (or an IgV domain) of IGSF11, or a variant thereof;
is able to enhance or increase (eg, enhances or increases) killing and/or lysis of tumour cells, preferably cancer cells or cells that originate from a tumour cell and/or cells that express IGSF11 or an IgC2 domain (or an IgV domain) of IGSF11, or a variant thereof;
is an anti-tumour antibody;
is a therapeutic antibody able to treat, ameliorate and/or delay progression of a disease, disorder or condition, in particular a disease, disorder or condition mentioned herein elsewhere;
is able to inhibit (eg, inhibits) tumour growth in-vivo, preferably in a murine model of cancer;
is able to inhibit (eg, inhibits) the binding of an interacting protein to IGSF11 protein or a variant thereof, suitably wherein: (i) the interacting protein is VSIR (VISTA) protein or a variant thereof; or, alternatively (ii) wherein the interacting protein is not VSIR (VISTA) protein or a variant thereof;
is able to inhibit (eg, inhibits) the interaction between VSIR (VISTA) protein or a variant thereof and the IgC2 domain (or the IgV domain) of IGSF11 protein or a variant thereof or, alternatively (ii) is not able to inhibit (eg, does not inhibit) the interaction between VSIR (VISTA) protein or a variant thereof and the IgC2 domain (or the IgV domain) of IGSF11 protein or a variant thereof;
is able to enhance (eg enhances) killing and/or lysis of cells expressing IGSF11, or a variant of IGSF11, by cytotoxic T cells and/or TIL;
is able to enhance (eg enhances) a cell-mediated immune response, such as that mediated by an activated cytotoxic T-cell (CTL), to a mammalian cell expressing said IGSF11 or the variant of IGSF11;
is able to increase (eg increases) immune cell, such as T-cell, activity and/or survival in the presence of a mammalian cell expressing said IGSF11 or the variant of IGSF11;
is able to modify (eg modifies) the microenvironment of a tumour, suitably increases the number and/or type of immune cells present in the tumour, and more suitably reduces the number of intra-tumoural MDSCs and/or increases the number of intra-tumoural CTLs;
decreases (the number of) M2 tumour-associated macrophages (TAMs) and/or increases the number of (intra-tumoural) CTLs, optionally, in each case, within the tumour microenvironment;
is able to recruit and/or activate (eg recruits and/or activates) NK cells and/or (to) mediate (eg mediates) antibody-dependent cellular cytotoxicity (ADCC);
is able to recruit and/or activate (eg recruits and/or activates) macrophages and/or (to) mediate (eg mediates) antibody-dependent cellular phagocytosis (ADCP);
is able to recruit (eg recruits) complement and/or (to) mediate (eg mediates) complement dependent cytotoxicity (CDC); and/or
is able to induce internalisation of (eg induces internalisation, or internalises) IGSF11 protein from the surface of cells (such as tumour cells that express IGSF11) (eg when the ABP is bound to cells (such as tumour cells) able to express (eg that expresses) IGSF11).

In any of such aspects (and eg as described in further detail elsewhere herein), the ABP can be an antibody, or an antigen binding fragment thereof. In particular, the antibody can be a monoclonal antibody, or wherein the antigen binding fragment can be a fragment of a monoclonal antibody. For example, such an antibody can be a human antibody a humanised antibody or a chimeric-human antibody, or the antigen binding fragment can be a fragment of a human antibody a humanised antibody or a chimeric-human antibody.

In any of such aspects (and eg as described in further detail elsewhere herein), the ABP can be expressed on the surface of an immune cell (such as T cell, eg an autologous T cell). In particular of such embodiments, the ABP may comprise and/or be expressed as a chimeric antigen receptor (CAR).

The invention also relates to an aspect, and as may be further described, defined, claimed or otherwise disclosed herein, being a method for (or of) inhibiting an interaction between IGSF11 protein (or a variant thereof) and an interacting protein of IGSF11 protein, such as an interacting protein that binds to an IgC2 domain of IGSF11 protein (or, in another aspect, that binds to an IgV domain of IGSF11 protein) or a variant thereof, the method comprising the step of:
exposing IGSF11 protein (or a variant thereof) to a compound that is an inhibitor of the expression, function, activity and/or stability of an IgC2 domain of IGSF11 protein (or, in the other aspect, is an inhibitor of the expression, function, activity and/or stability of an IgV domain of IGSF11 protein) or a variant thereof,

with the proviso that the compound is not an ABP that is the subject of one or more of the provisos (A), (B), (C), (D), (E) and/or (F) as set out elsewhere herein,

thereby, inhibiting the interaction between IGSF11 protein (or variant thereof) and an interacting protein of IGSF11 protein. Such a method can be practiced, in certain embodiments, as an in-vitro method.

The invention also relates to an additional aspect, and as may be further described, defined, claimed or otherwise disclosed herein, being a method for (or of) treating a subject in need thereof, said treatment comprising inhibiting the interaction between IGSF11 protein (or a variant thereof) and an interacting protein of IGSF11 protein, such as an interacting protein that binds to an IgC2 domain of the IGSF11 protein (or, in another aspect, that binds to an IgV domain of IGSF11 protein) or a variant thereof, the method comprising the step of:
administering to the subject a (eg, therapeutically effective amount of a) compound that is an inhibitor of the expression, function, activity and/or stability of an IgC2 domain of IGSF11 protein (or, in the other aspect, that is an inhibitor of the expression, function, activity and/or stability of an IgV domain of IGSF11 protein) or a variant thereof,

with the proviso that the compound is not an ABP that is the subject of one or more of the provisos (A), (B), (C), (D), (E) and/or (F) as set out elsewhere herein,

to inhibit the interaction between IGSF11 protein (or variant thereof) and an interacting protein of IGSF11 protein.

In a first aspect directed to ABPs, and as may be further described, defined, claimed or otherwise disclosed herein, the invention relates to an antigen binding protein (ABP) which specifically binds to a C2-type immunoglobulin-like (IgC2) domain of IGSF11 (VSIG3) protein and, optionally, wherein the ABP and is able to inhibit (eg, inhibits) the interaction between an interacting protein such as VSIR (VISTA) protein or a variant thereof to IGSF11 protein or a variant thereof (eg, to an IgC2 domain of IGSF11 protein or a variant of such domain). In an alternative first aspect, and as may be further described, defined, claimed or otherwise disclosed herein, the invention relates to an antigen binding protein (ABP) which specifically binds to a V-type immunoglobulin-like (IgV) domain of IGSF11 (VSIG3) protein and, optionally, wherein the ABP and is able to inhibit (eg, inhibits) the interaction between an interacting protein to IGSF11 protein or a variant thereof (eg, to a IgV domain of IGSF11 protein or a variant of such domain).

In certain embodiments the ABP is optionally able to inhibit (eg, inhibits) the binding of IGSF11 protein or a variant thereof to an interacting protein that is an endogenous binding partner of IGSF11 protein. For example, in one embodiment, the interacting protein is VSIR (VISTA) protein or a variant thereof. However, in other embodiments the interacting protein is another immunoglobulin superfamily member, such as VSIG8, or is a co-receptor of IGSF11 or a junctional protein (eg, a gap junction protein). In yet other embodiments, the interacting protein is a protein involved in formation, regulation and/or maintenance of an immune synapse, and/or a protein involved in immune synaptic transmission and/or plasticity, in each case such as between and immune cells and a tumour cell (eg a tumour cell that expresses IGSF11).

Antigen Binding Proteins Targeting the IgC2 Domain of (or the IgV Domain of) IGSF11

An “antigen binding protein” (“ABP”) as used herein means a protein that specifically binds to a target antigen, such as to one or more epitope(s) displayed by or present on a target antigen. The antigen of the ABPs of the invention is (or is comprised in) the IgC2 domain of IGSF11 or an orthologue (or paralogue) or other variant thereof; or in an alternative aspect, the antigen of the ABPs of the invention is (or is comprised in) the IgV domain of IGSF11 or an orthologue (or paralogue) or other variant thereof; (such as the epitope(s) can be displayed by or present on such domain of said IGSF11 or variant). Typically, an antigen binding protein is an antibody (or a fragment thereof), however other forms of antigen binding protein are also envisioned by the invention. For example, the ABP may be another (non-antibody) receptor protein derived from small and robust non-immunoglobulin “scaffolds”, such as those equipped with binding functions for example by using methods of combinatorial protein design (Gebauer & Skerra, 2009; Curr Opin Chem Biol, 13:245). Particular examples of such non-antibody ABPs include: Affibody molecules based on the Z domain of Protein A (Nygren, 2008; FEBS J 275:2668); Affilins based on gamma-B crystalline and/or ubiquitin (Ebersbach et al, 2007; J Mo Biol, 372:172); Affimers based on cystatin (Johnson et al, 2012; Anal Chem 84:6553); Affitins based on Sac7d from Sulfolobus acidcaldarius (Krehenbrink et al, 2008; J Mol Biol 383:1058); Alphabodies based on a triple helix coiled coil (Desmet et al, 2014; Nature Comms 5:5237); Anticalins based on lipocalins (Skerra, 2008; FEBS J 275:2677); Avimers based on A domains of various membrane receptors (Silverman et al, 2005; Nat Biotechnol 23:1556); DARPins based on an ankyrin repeat motif (Strumpp et al, 2008; Drug Discov Today, 13:695); Fynomers based on an SH3 domain of Fyn (Grabulovski et al, 2007; J Biol Chem 282:3196); Kunitz domain peptides based on Kunitz domains of various protease inhibitors (Nixon et al, Curr opin Drug Discov Devel, 9:261) and Centyrins and Monobodies based on a 10th type III domain of fibronectin (Diem et al., 2014; Protein Eng Des Sel 27:419 doi: 10.1093/protein/gzu016; Koide & Koide, 2007; Methods Mol Biol 352:95). In the context of an ABP of the present invention that specifically binds IGSF11, such an ABP is not a protein being V-set immunoregulatory receptor “VSIR” (or VISTA), or a IGSF11-binding fragment or other variant of VSIR (VISTA) (in particular, a variant having more than 70%, 80% or 90% sequence identify to the amino acid sequence of human VSIR (VISTA).

The term “epitope” includes any determinant capable of being bound by an antigen binding protein, such as an antibody. An epitope is a region of an antigen that is bound by an antigen binding protein that targets that antigen, and when the antigen is a protein, includes specific amino acids that bind the antigen binding protein (such as via an antigen binding domain of said protein). Epitope determinants can include chemically active surface groupings of molecules such as amino acids, sugar side chains, phosphoryl or sulfonyl groups, and can have specific three dimensional structural characteristics, and/or specific charge characteristics. Generally, antigen binding proteins specific for a particular target antigen will preferentially recognise an epitope on the target antigen in a complex mixture of proteins and/or macromolecules. Epitopes of or within (eg comprised in) a target antigen may be: (i) continuous epitopes, which typically are linear sequences of amino acids and/or the surface groupings of a linear sequences of amino acids; or (ii) discontinuous epitopes, which typically exist only when the protein is folded into a particular conformation. For example, a discontinuous epitope, as referred to herein, may be understood as at least two non-adjacent amino acid sequence stretches within a given polypeptide chain which are simultaneously and specifically (as defined above) bound by one antibody molecule.

The term “extracellular domain” (“ECD” or “EC” domain) as used herein refers to the region or regions of the protein which are exposed to the extracellular space and which are typically responsible for ligand binding. Immunoglobulin (Ig) superfamily genes typically have an Immunoglobulin-like ECD, such as an Ig-like V-type domain.

An antigen binding protein is “specific” when it binds to one antigen (such as IGSF11; eg human IGSF11, orthologues and other variants thereof) more preferentially (eg, more strongly or more extensively) than it binds to a second antigen. The term “specifically binds” (or “binds specifically” and the like) used herein in the context of an ABP means that said ABP will preferentially bind to the desired antigen (eg IGSF11, in particular a domain of an ECD of IGSF11 such as an IgC2 (or IgV) domain of IGSF11) than to bind to other proteins (or other molecules), such as preferentially binding to such IGSF11 or such domain compared to one or more of other Immunoglobulin (Ig) superfamily genes or to one or more of the Ig-like domains, such as an IgV (or IgC2) domain of IGSF11. Therefore, preferably, the binding affinity of the ABP to the one antigen (e.g. IGSF11) is at least 2-fold, 5-fold, at least 10-fold, at least 20-fold, at least 50-fold, at least 100-fold, at least 200-fold, at least 500-fold, at least 1000-fold, at least 2000-fold, at least 5000-fold, at least 10000-fold, at least 10⁵-fold or even at least 10⁶-fold, most preferably at least 2-fold, compared to its affinity to the other targets (e.g. unrelated proteins such as mouse or human Fc domain, or streptavidin). Therefore, in one particularly preferred embodiment, the binding affinity of the ABP to the one antigen being an IgC2 domain of IGSF11 (or an IgV domain of IGSF11) is at least 2-fold, 5-fold, at least 10-fold, at least 20-fold, at least 50-fold, at least 100-fold, at least 200-fold, at least 500-fold, at least 1000-fold, at least 2000-fold, at least 5000-fold, at least 10000-fold, at least 10⁵-fold or even at least 10⁶-fold, most preferably at least 2-fold, compared to its affinity to other targets (e.g. unrelated proteins such as mouse or human Fc domain, or streptavidin) and/or antigens such as another domain of IGSF11, for example an IgV domain of IGSF11 (or, an IgC2 domain of IGSF11).

Immunoglobulin superfamily member 11 (IGSF11)—also known as Brain and testis-specific immunoglobulin superfamily protein (Bt-IGSF or BTIGSE), V-set and immunoglobulin domain-containing protein 3 (VSIG3) and coxsackie virus and adenovirus receptor-like 1 (CXADRL1)—was first described by Suzu et al (2002; Biochem Biophys Res Comm 296:1215) as “BT-IgSF”, a novel gene from both human and mouse that encoded a new member of the immunoglobulin superfamily that was preferentially expressed in both brain and testis. (hence, BT-IgSF: brain- and testis-specific immunoglobulin superfamily), and that showed significant homology to coxsackie and adenovirus receptor (CAR) and endothelial cell-selective adhesion molecule (ESAM). Human Bt-IgSF protein (431 amino acids) was described to be 88% identical to the mouse protein (428 amino acids). The human gene and protein from such research was the subject of at least EP1321475 (eg SEQ ID NOs 1 and 2 thereof), describing a gene useful for diagnosis and treatment of aplasia of corpus callosum and aspermatogensis and use thereof. However, alternative variants of the same sequence were the subject of earlier patent applications based on the predicted protein sequence from large-scale cDNA-sequencing (eg, 422 amino acids, SEQ ID NO 667 of WO2001/54474; 431 amino acids, SEQ ID NO 22 of WO2003/027228) as well as in later analogous large-scale projects (eg 428 amino acids, SEQ ID NO 4,513 of US 2004/0005560). Katoa & Katoa (2003; Int J Onc 23:525) described two isoforms of the IGSF11 gene (differing in the first 17 amino acids), that the gene encoded adhesion molecules homologous to CXADR, FLJ22415 and ESAM, and was frequently up-regulated in intestinal-type gastric cancer; further suggesting that IGSF11 might be a target for early diagnosis (eg by antibodies) of intestinal-type gastric cancer as well as for drug delivery to cancer cells. Harada et al (2005; J Cell Physiol 204:919) described BT-IgSF as a novel immunoglobulin superfamily protein that mediated homophilic adhesion in a calcium-independent manner.

Suppression of IGSF11 (VSIG3) by siRNA retarded the growth of gastric cancer cells, suggesting that IGSF11 (VSIG3) is a good candidate for cancer immunotherapy using IGSF11 peptides as cancer antigen, in particular for cancers of the stomach, colon and liver (Watanabe et al, 2005; Cancer Sci 96:498; WO2003/104275 and SEQ ID NO 2 thereof, 431 amino acids). WO2004/022594 described the cloning of the human second isoform of IGSF11 (eg, SEQ ID NO 6 thereof, 430 amino acids; and designated such protein therein as “B7-H5” [note, this was a patent nomenclature, and not to be confused with the “B7-H5” used as a synonym for VSIR]) and production of soluble (secreted) forms of human and mouse such “B7-H5”. In particular, Example 13 of WO2004/022594 described the stimulation of B cell proliferation but not T cell proliferation by such mouse “B7-H5”, Examples 15 and 16 described modulation of B cells in vivo following administration of such murine “B7-H5-Fc” fusion protein, and further prophetic examples of WO2004/022594 postulated other immunologic effects of such “B7-H5” including in therapy.

Recently, IGSF11 (BT-IgSF) was described to play a major role in male fertility in mice (Pelz et al 2017, J Biol Chem 292:21490). This study demonstrated that the absence of BT-IgSF in Sertoli cells in both global and conditional mouse mutants resulted in male infertility, atrophic testes with vacuolation, azoospermia, and spermatogenesis arrest. Although transcripts of certain junctional proteins were up-regulated in the absence of BT-IgSF, the functional integrity of the blood-testis barrier was impaired. In neuronal development, IGSF11 has been shown to regulate synaptic transmission and plasticity through its interaction with certain scaffolding proteins and neurotransmitter receptors (Jang et al, 2016; Nat Neurosci 19:84).

An extensive functional ELISA binding screening assay revealed that IGSF11 (VSIG3) binds the (eg, IGSF11 interacts with) B7 family member V-set immunoregulatory receptor (VSIR) (which was initially described and designated as “V-domain Ig suppressor of T cell activation” (VISTA)) but did not interact with other known members of the B7 family (Wang et al, 2017; J Immunol 198 [1 Supplement] 154.1, poster published 2016 Wang et al 2018, Immunology 156:74). VSIR (VSIG3) was described therein to inhibit human T cell proliferation in the presence of T cell receptor signalling, and to significantly reduce cytokine and certain chemokine production by human T cells. Furthermore, anti-VISTA neutralisation antibodies attenuated the binding of IGSF11 (VSIG3) to VSIR (VISTA), as well as VSIR-induced T cell inhibition. Thus, Wang et al proposed that they had identified a novel B7 pathway able to inhibit human T cell proliferation and cytokine production, and that this IGSF11/VSIR (VSIG3/VISTA) co-inhibitory pathway may provide new strategies for the treatment of human cancers, autoimmune disorders, infection, and transplant rejection, and may help to design better vaccines.

The interaction between IGSF11 (VSIG3) and VSIR (VISTA) has subsequently been independently described using a high throughput screen for receptor parings (Yang et al, 2017; J Biotech http://dx.doi.org/101016/j.jbiotec.201708023). Yang et al speculated that cancer cells utilise IGSF11 to supress the activation of T cells and escape the immune surveillance of immune cells, and further that IGSF11 may be a potential target for cancer immunotherapy due to it being a binding partner for VSIR (VISTA).

WO 2018/027042 A1 describes that the ligand for VISTA (VSIR) is identified as VSIG3 (IGSF11). This disclosure predicts that in the interaction between IGSF11 (VSIG3) and VSIR (VISTA), only their N-terminal domains are involved, and their intercellular binding is mediated by the IgV domain of IGSF11 (VSIG3), with a 4:2 stoichiometry between VSIR (VISTA) and IGSF11 (VSIG3). The “GFC” Ig beta-sandwich front face of the IgV domain of IGSF11 (VSIG3) is indicated as being involved with the interaction with VSIR (VISTA) and the “ABE” Ig beta-sandwich back face of the IgV domain of IGSF11 (VSIG3) is indicated as being involved with either homodimerisation between IGSF11 (VSIG3) molecules or between IGSF11-VSIG8V heterodimerisation (see FIGS. 17A and 17B of WO 2018/027042 A1). Indeed, WO 2018/027042 A1 describes only two distinct ways to block assembly of a IGSF11 (VSIG3) and VSIR (VISTA) interaction by anti-IGSF11 antibodies (see FIG. 17E of WO 2018/027042 A1): (1) antibodies binding to the IgV domain of IGSF11 that blocking the GFC front-front IGSF11-VISTA interaction; or (2) antibodies binding to the IgV domain of IGSF11 that blocking ABE back-back IGSF11-IGSF11 homodimerisation (or IGSF11-VSIG8V heterodimerisation). In particular, WO 2018/027042 A1 especially describes embodiments of anti-IGSF11 antibodies that interact with an ABE Ig face, or with an GFC Ig face, of IGSF11 (see [00151 of WO 2018/027042 A1); such faces being present in the N-terminal Ig-like V-type domain of IGSF11 (VSIG3).

The key role of the GFC face (of the Ig-like V-type domain) in the interaction between IGSF11 (VSIG3) and VSIR (VISTA) as speculated was supported by the general understanding in the art that such GFC face-mediated Ig domain interactions are the most common way for Ig domains to bind, and have been captured by X-ray crystallography, in nearly every minimal binding complex between cell surface immunoregulatory receptors (Stengel et al 2012), and even antibodies and T-cell receptor (TCR) complexes (Lin et al, 2008, as further supported in WO 2018/027042 with FIGS. 17C and 17D therein depicting the GFC face-directed interactions of PVR-TIGIT and PD1-PDL1/PDL2, respectively). WO 2018/027042 would appear not to suggest a role of the IgC2 domain of IGSF11 (VSIG3) in its interactions with other molecules, and limits mention of the IgC2 domain anecdotally as a portion of IGSF11 that may be being present in an ECD of IGSF11 (VSIG3) (eg, [00135] of WO 2018/027042). Of note, however, the inventors later published experiments that question if their assays have any domain specificity (Wang et al 2019): suggesting that both the IgV and IgC2 domains of IGSF1 (VSIG3), as Fc fusion proteins, bound to human VSIR (VISTA) in a functional ELISA assay and in a co-immunoprecipitation assay from human PBMC lysates.

Indeed, the role of V-type domains in intercellular binding between immunoglobulin superfamily receptor/ligand pairs has been generally accepted and widely described, including for several immunoglobulin superfamily receptor/ligand pairs involved in tumour cell immune evasion, such as: (i) PD1 interacting with PDL1 or PDL2 (eg, Lin et al 2008; Lazar-Molnar et al 2009); (ii) CD80 interacting with CD28 or CTLA4 (eg, Sanchez-Lockhart et al 2014; Stamper et al 2001); and (iii) CD86 interacting with CD28 or CTLA4 (eg, Rennert et al 1997). Hence, considerable prior art teaches that Ig-like V-type domains are those which are (almost exclusively) involved in intercellular (trans) interactions between immunoglobulin superfamily members (including, in particular, IGSF11), and can also be involved in homo- and heterodimerisation between such immunoglobulin superfamily members in cis-interactions.

There is some evidence that IgC domains are involved in cis-interactions of immunoglobin superfamily members, for example homodimerisation of: (i) SIRPalpha (Lee et al 2010, J Biol Chem 285:37953); (ii) CD80 (Girard et al 2014, Immunol Lett 161:65); (iii) CD86 (Girard et al 2014, Immunol Lett 161:65); and (iv) CD277 (Plaakodeti et al 2012, J Biol Chem 287:32780). In particular, although homophilic adhesion of the human cell adhesion molecule CEACAM1 directly involves the N-terminal domain (eg IgV domain), this is only possible if the IgC domains are present (Watt et al, 2001; Blood 98:1469).

“Immunoglobulin superfamily member 11”- or “IGSF11” (or “VSIG3”)—as a protein is, in the context of the invention, an immunoglobulin superfamily member and, typically, one that is capable of binding (eg binds) to one or more interacting protein (in particular to endogenous binding partners), such as those described herein VSIR (VISTA). Pertinent information on the human IGSF11 gene is found at Entrez Gene ID: 152404; HGNC ID:16669; Genome Coordinates for assembly GRCh38:CM000665.2: Chromosome 3: 118,900,557-119,146,068 reverse strand, and information on human IGSF11 protein is accessible on UniProt: Q5DX21 (eg, Entry version 115 of 25 Oct. 2017). An IGSF11 protein in the context of the invention has, typically, the domain structure shown in FIG. 1A, and preferably (eg as a human IGSF11 protein) comprises an amino acid sequence of one of its isoforms as shown in any of SEQ ID NOs: 371 to 373, more preferably SEQ ID NOs. 371 or 372. With reference to SEQ ID NO. 371, amino acids 1 to 22 represent an N-terminal signal peptide, amino acids 23 to 241 form the extra cellular domain (SEQ ID NO. 374), amino acids 242 to 262 form the helical transmembrane (TM) region and amino acids 263 to 431 form the cytoplasmic domain. As described in more detail elsewhere herein, the extracellular domain (ECD) of (human) IGSF11 forms two (sub)domains, with amino acids 23 to 136 (SEQ ID NO. 375) forming an Ig-like V-type domain, and amino acids 144 to 234 (SEQ ID NO. 376) forming an Ig-like C2-type domain.

“IgV domain” (or “Ig-like V domain”) and “IgC domain” (or “Ig-like C domain”) as used herein, refer broadly to Ig superfamily member domains. These domains correspond to structural units that have distinct folding patterns called Ig-like folds. Ig-like folds are comprised of a sandwich of two sheets of antiparallel beta strands, with a conserved disulphide bond between the two sheets in most, but not all, domains. IgC domains of Ig, TCR, and MHC molecules share the same types of sequence patterns and are called the C1 set domains within the Ig superfamily. Other IgC domains fall within the IgC2 set domain (an “IgC2-type domain” (or Ig-like C2 domain” or “C2-type Ig-like domain” or the like)). IgV domains also share sequence patterns and are called V set domains. In particular, an IgC2 domain of human IGSF11 encompasses from about amino acid 144 to about amino acid 234 of the amino acid sequence of human IGSF11 (UniProt: Q5DX21 (eg, Entry version 115 of 25 Oct. 2017); and an V-type immunoglobulin-like (IgV) domain of human IGSF11 encompasses from about amino acid 23 to about amino acid 136 of such amino acid sequence of human IGSF11. Also, what is considered to be a given “domain” of a protein (eg, the ECD, IgV domain or IgC2 domain of IGSF11) may vary by one, two, three, four or more amino acids at the amino end, the carboxyl end, or both ends of any of the stretches of amino acids described herein An IgC2 domain of human IGSF11 may, in certain embodiments, include the short stretch of amino acids (eg, approximately 7 amino acids) up to an IgV domain, such that an IgC2 domain of human IGSF11 can begin from about amino acid 137 of the amino acid sequence of human IGSF11, and/or may comprise amino acids in the sequence of human IGSF11 up to the TM domain, such that an IgC2 domain of human IGSF11 can end at about amino acid 241 of the amino acid sequence of human IGSF11. An IgV domain of human IGSF11 may, in certain embodiments, include the short stretch of amino acids up to an IgC2 domains, such that an IgV domain of human IGSF11 can end at about amino acid 143 of the amino acid sequence of human IGSF11. In one particular embodiment, an IgC2 domain of human IGSF11 encompasses from about acid 137 to about amino acid 241 of the amino acid sequence of human IGSF11 (SEQ ID NO. 388). In one particular embodiment, an IgV domain of human IGSF11 encompasses from about acid 23 to about amino acid 143 of the amino acid sequence of human IGSF11 (SEQ ID NO. 389). Wang et al, 2018 (Immunology 156:74) describe the “C-type immunoglobulin-like domain” of human IGSF11 to fall between amino acids 144 and 241 (SEQ ID NO. 390), and mark the various regions of human IGSF11 as shown in FIG. 1B. In all aspects of the invention (and/or embodiments thereof) that do not explicitly specify the domain of IGSF11 (such as those using the phrase “IGSF11-domain” or using the phrase “domain” in the context of “IGSF/domain” or “IGSF, domain . . . ”, are included: (1) embodiments where such domain of IGSF11 is a IgC2 domain of IGSF11; and (2) other embodiments where such domain of IGSF11 is a IgV domain of IGSF11.

The human IGSF11 gene is located at chromosomal position 3q13.32, and has orthologues (eg, is conserved) in many species such as in chimpanzee and other great apes, Rhesus, Cynomolgus and green monkeys, marmoset, dog, pig, cow, mouse etc. In particular, the amino acid sequence for the IGSF11 protein in Cynomolgus monkey (UniProt identifier G7NXN0, Entry version 14 of 25 Oct. 2017; 97.0% identical to human) is as shown in SEQ ID NO. 377 and in mouse (UniProt identifier POC673, Entry version 78 of 25 Oct. 2017; 88.4% identical to human) is shown in SEQ ID NO. 378. The closest human paralogue to human IGSF11 is coxsackievirus and adenovirus receptor, CXAR (33.3% identity to human IGSF11). The term IGSF11 in some embodiments of the invention may also pertain to variants of the human IGSF11 protein having an amino acid sequence that is substantially identical to, or of at least 70%, 75% or 80%, preferably 85%, more preferably at least 90%, 95%, 96%, 97%, 98%, 99%, or 100% sequence identity (such as at least 90% or 95% sequence identity) to, the amino acid sequence shown in any of SEQ ID NOs. 371 to 373, as determined using, e.g., the “Blast 2 sequences” algorithm described by Tatusova & Madden 1999 (FEMS Microbiol Lett 174: 247-250), and which (preferably) retain biological activity identical or substantially identical to the respective reference IGSF11 (eg to bind to VSIR (VISTA) protein and/or to suppress T cell (or other immune cell) function/activity. Preferred variants of IGSF11 protein comprise sequence variants thereof due to sequence polymorphism between and within populations of the respective species, as well as mutations compared to the wild-type sequence of IGSF11 (eg SEQ ID NO. 371). A preferred variant of IGSF11 protein is an IGSF11 variant (compared to eg SEQ ID NO. 371) selected from the list consisting of: P39T (corresponding to variant dbSNP:rs2903250), E333D (corresponding to variant dbSNP:rs36052974) and S388N (corresponding to variant dbSNP:rs34908332). The term IGSF11 can mean, as applicable to the context (if not more specifically indicated), an IGSF11 protein (such as one described above) or an mRNA molecule encoding such an IGSF11 protein.

In certain of embodiments, an ABP of the invention that binds to an IgC2 domain of (or, in the alternative aspect, to an IgV domain of) human IGSF11 protein is cross reactive to an IgC2 domain of (or, in the alternative aspect, to an IgV domain of) an orthologous protein, such as cross reactive to an IgC2 domain of (or, in the alternative aspect, to an IgV domain of) cynomolgus IGSF11 protein and/or to an IgC2 domain of (or, in the alternative aspect, to an IgV domain of) mouse IGSF11 protein and/or to an IgC2 domain of (or, in the alternative aspect, to an IgV domain of) rat IGSF11 protein.

The term “orthologue” as used herein means a variant that descends from the same ancestral gene but which is present in another organism due to a speciation event. Orthologues of IGSF11, or domains thereof, are typically expected to retain the same function as (or have a similar function to) human IGSF11 or such domain. Those orthologues of human IGSF11 include those of chimpanzee (431 amino acids; 99.3% identity), cow (437 amino acids; 91.1% identity), mouse (428 amino acids; 88.4% identity) and rat (428 amino acids; 88.9% identity). A particular orthologue of human IGSF11 is that of cynomolgus monkeys or of mouse. An example of a cynomolgus monkey orthologue of human IGSF11 is described above, and of a mouse orthologue of human IGSF11 is described above.

The term “paralogue” as used herein means a variant in the same organism that descends from the same ancestral gene by a duplication event. A paralogue of IGSF11 is typically expected to be an immunoglobulin superfamily protein, in particular one having at least 70%, 80% 85% or 90% sequence identity to the amino acid sequence of the IGSF11 (if any such a paralogue exists in humans).

The term “variant” as used herein in the context of a protein (or domain thereof) means any natural or non-natural version of such protein (or of such domain) which comprises one or more amino acid mutations compared to the reference protein (or to reference domain), but which shares significant amino acid sequence identity with the reference protein (or with the reference domain), e.g. at least 70% or 75% amino acid sequence identity, preferably at least 80% amino acid sequence identity, more preferably at least 90% amino acid sequence identity and most preferably at least 95%, 96%, 97%, 98% or 99% amino acid sequence identity. Preferably, the variant of the protein (or the domain) possesses and/or maintains at least one function/activity that is the same, essentially the same or similar as the reference protein (or as the reference domain). Variants of IGSF11 may include orthologues to and natural variants of human IGSF11, such as the natural variants P39T, E333D and S388N, and others variants such as Y267H, V374A and K395E. Variants of IGSF11 may also correspond to human IGSF11 with one or more amino acid residues inserted into, or deleted from the amino acid sequence, such as those variants of IGSF11 naturally found within a population or those made by genetic manipulation, such as to specifically engineer amino acid changes into one or more domains (such as extracellular domains) of the variant. Variants of IGSF11 include fusion proteins of IGSF11 (for example, a human IGSF11 fused to a heterologous polypeptide chain, such as Fc immunoglobulin domains or tags), and/or IGSF11 conjugated to another chemical moiety such as an effector group or a labelling group. A variant of IGSF11 can, in certain embodiments, comprise a fragment of IGSF11, for example a polypeptide that consists of one or more EC domains (or regions or (sub)domains thereof) of IGSF11 without one or other (or any other) EC, TM or intracellular domains of IGSF11. Preferred such variants of IGSF11 that are fragments include those that comprise an ECD of IGSF11 without any of the TM or intracellular domains of IGSF11; more preferably those that comprise the Ig-like V-type domain (SEQ ID NO. 375), or the Ig-like C2-type domain (SEQ ID NO. 376,) of IGSF11 without one or more (or all) of the other ECD, TM or intracellular domains of IGSF11. Variants of IGSF11 also include versions of human IGSF11 (or orthologues thereof) that have been modified to display only specific domains (such as extracellular), or not to display one or more other domains, and/or to display certain domains (e.g. ECDs) of human IGSF11 in combination with domains from paralogues and/or orthologues of human IGSF11, or from other immunoglobulin superfamily proteins. Methods describing the engineering of domain or amino acid variants of IGSF11 are known to the person of ordinary skill. In certain embodiments, the variant of IGSF11 is a functional variant thereof. A “functional variant” of IGSF11 (such as a functional domain or fragment of an IGSF11 protein) is a variant of the protein of IGSF11 that provides, possesses and/or maintains one or more of the herein described functions/activities of the non-variant protein (or domain) of IGSF11. For example, such functional variant may bind an interacting protein of IGSF11 (eg VSIR (VISTA) protein) and/or to suppress T cell (or other immune cell) function/activity as IGSF11 protein (or domain thereof), such as having the same, essentially the same or similar specificity and/or function as a receptor as IGSF11 protein (or as the domain thereof). In other embodiments, such a functional variant may possess other activities than those possessed by the non-variant IGSF11 protein (or domain thereof), as long as, preferably, it provides, possesses and/or maintains at least one function/activity that is the same, essentially the same or similar as IGSF11 protein (or domain thereof). In more preferred embodiments, a functional variant of IGSF11 (or domain thereof) may act as an immune checkpoint inhibitor, such as by inhibiting one or more cell-based immune response(s) to a tumour or cancer cell that expresses such functional variant.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm.

A standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) may also be used by the algorithm.

Examples of parameters that can be employed in determining percent identity for polypeptides or nucleotide sequences using the GAP program are the following: (i) Algorithm: Needleman et al., 1970, J. Mol. Biol. 48:443-453; (ii) Comparison matrix: BLOSUM 62 from Henikoff et al., 1992, supra; (iii) Gap Penalty: 12 (but with no penalty for end gaps); (iv) Gap Length Penalty: 4; (v) Threshold of Similarity: 0.

A preferred method of determining similarity between a protein or nucleic acid and (or between) human IGSF11, a paralogue, orthologue or other variant thereof (such as a domain of IGSF11), is that provided by the Blast searches supported at Uniprot supra (e.g., http://www.uniprot.org/uniprot/Q5DX21); in particular for amino acid identity, those using the following parameters: Program: blastp; Matrix: blosum62; Threshold: 10; Filtered: false; Gapped: true; Maximum number of hits reported: 250.

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least about 10, 15, 20, 25, 30, 35, 40, 45, 50 or other number of contiguous amino acids of the target polypeptide or region thereof.

In particular embodiments of the invention, the IGSF11 is human IGSF11, preferably a protein comprising an amino acid sequence selected from the group consisting of: SEQ ID NO: 371, SEQ ID NO: 342 and SEQ ID NO: 343 (in particular, SEQ ID NO. 371), or a protein having no more than two, four, six, eight, or ten, for example no more than one, two or three, such as no more than one, amino acid substitutions, insertions or deletions compared to these sequences.

In the context of variants of IGSF11, the invention includes those embodiments where a variant of IGSF11 is a protein comprising an amino acid sequence having at least 80%, 85%, 90%, 92% 95% or 97% sequence identity (in particular, at least 92% or 95% sequence identity) to the sequence of SEQ ID NO: 371.

In the context of other variants of IGSF11 (or domain thereof), the invention also includes those embodiments where a variant of IGSF11 is selected from the group consisting of an ortholog (or paralog) of IGSF11, and a functional fragment of an IGSF11 protein (or domain thereof). In certain of such embodiments, such functional fragment of an IGSF11 protein (or domain thereof, such as an IgC2 or an IgV domain of IGSF11) binds to an interacting protein of IGSF11 (for example to a VSIR (VISTA) protein), such as a human VSIR protein, or a variant of VSIR (such as one described elsewhere herein) or to another interacting protein as described elsewhere herein. In another of such embodiments, such functional fragment of an IGSF11 protein (or domain thereof) is capable of inhibiting (eg inhibits) a cell-based immune response to a cell, such as a cancer cell, that expresses such functional fragment. In particular of such embodiments, the variant of IGSF11 comprises at least a fragment of an extracellular domain (ECD) of an IGSF11 protein, such as of an ECD of a human IGSF11 protein and/or where the variant of VSIR protein is a functional fragment of a VSIR protein such as comprising an ECD of VSIR protein. For example, the variant of IGSF11 comprises an IgC2 domain of (human) IGSF11 (and/or the variant of IGSF11 comprises an IgV domain of (human) IGSF11).

In particular embodiments of the present invention, an extracellular domain (ECD) of IGSF11 is an ECD of human IGSF11 protein, such as wherein the ECD of a human IGSF11 protein is an amino acid sequence selected from the group consisting of: SEQ ID NO: 374, SEQ ID NO: 375 and SEQ ID NO: 376 (preferably, SEQ ID NO: 375), or an amino acid sequence having at least 80%, 85%, 90%, 92%, 95% or 97% sequence identity (preferably, at least 92% or 95% sequence identity) to these sequences, and/or having no more than two, four, six or eight, for example no more than one, two or three, such as no more than one, amino acid substitutions, insertions or deletions compared to these sequences.

In particular embodiments of the present invention, an IgC2 domain of IGSF11 is an IgC2 of human IGSF11 protein, such as wherein the IgC2 of a human IGSF11 protein is an amino acid sequence selected from the group consisting of: SEQ ID NO: 376, SEQ ID NO: 388 and SEQ ID NO: 390 (preferably, SEQ ID NO: 388), or an amino acid sequence having at least 80%, 85%, 90%, 92%, 95% or 97% sequence identity (preferably, at least 92% or 95% sequence identity) to these sequences, and/or having no more than two, four, six or eight, for example no more than one, two or three, such as no more than one, amino acid substitutions, insertions or deletions compared to these sequences.

In particular embodiments of the present invention, an IgV domain of IGSF11 is an IgV of human IGSF11 protein, such as wherein the IgV of a human IGSF11 protein is an amino acid sequence selected from the group consisting of: SEQ ID NO: 375 and SEQ ID NO: 389 (preferably, SEQ ID NO: 389), or an amino acid sequence having at least 80%, 85%, 90%, 92%, 95% or 97% sequence identity (preferably, at least 92% or 95% sequence identity) to these sequences, and/or having no more than two, four, six or eight, for example no more than one, two or three, such as no more than one, amino acid substitutions, insertions or deletions compared to these sequences.

An ABP of the invention may, in particular embodiments, be able to inhibit (eg, inhibits) the interaction between IGSF11 and an interacting protein to IGSF11 For example, such interacting partner may be: (i) an endogenous binding (protein) partner of IGSF11 (or a fragment or variant of such endogenous binding partner); or (ii) a biochemical binding (protein) partner, ie one that binds IGSF11 in a biochemical assay. In one embodiment, the interacting protein is VSIR (VISTA) protein or a variant thereof and IGSF11 protein or a variant thereof. For example, the ABP is optionally able to inhibit (eg, inhibits) the binding of IGSF11 protein or a variant thereof to the interacting protein (eg, VSIR (VISTA) protein or a variant thereof). Without being bound by theory, such an ABP of the invention may, by specifically binding to regions of the (eg ECD of) IGSF11 involved in the inter-molecular binding or complex formed between IGSF11 and the interacting protein (eg, VSIR), “block” the interaction between IGSF11 and the interacting protein (eg, VSIR). Accordingly, such an ABP of the invention can, in some embodiments, be a blocking ABP.

Information on V-set immunoregulatory receptor (VSIR), initially described and designated as “V-domain Ig suppressor of T cell activation” (VISTA) by Wang et al (2011), is described above, and “V-set immunoregulatory receptor”- or “VSIR” (or “VISTA”)—as a protein is, in the context of the invention, an immunoglobulin superfamily member and, typically, one that is capable of binding (eg binds) to IGSF11 (VSIG3). Pertinent information on the human VSIR gene is found at Entrez Gene ID: 64115; HGNC ID: 30085; Genome Coordinates for assembly GRCh38:CM000672.2: Chromosome 10: 71,747,559-71,773,498 reverse strand, and information on human VSIR protein is accessible on UniProt: Q9H7M9 (eg, Entry version 129 of 25 Oct. 2017) A VSIR protein in the context of the invention, typically, is approximately 50 kDa, is a type I transmembrane protein and has one IgV domain. Preferably (eg as a human VSIR protein) comprises an amino acid sequence as shown in SEQ ID NOs: 379. With reference to SEQ ID NO. 379, amino acids 1 to 32 represent an N-terminal signal peptide, amino acids 33 to 194 form the extra cellular domain (SEQ ID NO. 380), amino acids 195 to 215 form the helical transmembrane (TM) region and amino acids 216 to 311 form the cytoplasmic domain. The extracellular domain (ECD) of (human) VSIR forms an Ig-like V-type domain between amino acids 33 to 168 (SEQ ID NO. 381).

The human VSIR gene is located at chromosomal position 10q22.1, and has orthologues (eg, is conserved) in many species such as in chimpanzee and other great apes, Rhesus, Cynomolgus and green monkeys, marmoset, dog, pig, cow, mouse etc. In particular, the amino acid sequence for the VSIR protein in mouse (UniProt identifier Q9D659, Entry version 122 of 20 Dec. 2017; 77.2% identical to human) is shown in SEQ ID NO. 383. The closest human paralogue to human VSIR is programmed cell death 1 ligand 1, CD274 or PD-L1 (24.8% identity to human VSIR). The term VSIR in some embodiments of the invention may also pertain to variants of the human VSIR protein having an amino acid sequence that is substantially identical to, or of at least 70%, 75% or 80%, preferably 85%, more preferably at least 90%, 95%, 96%, 97% 98%, 99% or 100% sequence identity (such as at least 90% or 95% sequence identity) to, the amino acid sequence shown in SEQ ID NO. 379, as determined using, e.g., an algorithm described elsewhere herein, and which (preferably) retain biological activity identical or substantially identical to the respective reference VSIR (eg to bind to IGSF11 (VSIG3) protein and/or to suppress T cell (or other immune cel) function/activity). Preferred variants of VSIR protein comprise sequence variants thereof due to sequence polymorphism between and within populations of the respective species, as well as mutations compared to the wild-type sequence of IGSF11 (eg SEQ ID NO. 379). A preferred variant of VSIR protein is an VSIR variant (compared to eg SEQ ID NO. 379) D187E (corresponding to variant dbSNP:rs3747869). The term VSIR can mean, as applicable to the context (if not more specifically indicated), an VSIR protein (such as one described above) or an mRNA molecule encoding such an VSIR protein.

In particular embodiments of the invention, the VSIR is human VSIR, preferably a protein comprising an amino acid sequence of: SEQ ID NO: 379, or a protein having no more than two, four, six or eight, for example no more than one, two or three, such as no more than one, amino acid substitutions, insertions or deletions compared to this sequence

In the context of variants of VSIR, the invention includes those embodiments where a variant of VSIR is a protein comprising an amino acid sequence having at least 80%, 85%, 90%, 92% 95% or 97% sequence identity (in particular, at least 92% or 95% sequence identity) to the sequence of SEQ ID NO: 379.

In the context of other variants of VSIR, the invention also includes those embodiments where a variant of VSIR is selected from the group consisting of an ortholog (or paralog) of VSIR, and a functional fragment of a VSIR protein, preferably where such functional fragment of a VSIR protein binds to IGSF11 (VSIG3), such as a human IGSF11 protein, or a variant of IGSF11, and/or where such functional fragment of a VSIR protein functions as an immune checkpoint. In particular of such embodiments, the variant of VSIR comprises an extracellular domain (ECD) of an VSIR protein, such as an ECD of a human VSIR protein and/or where the variant of IGSF11 protein is a functional fragment of a IGSF11 protein such as comprising an ECD of IGSF11 protein.

In particular embodiments of the present invention, an extracellular domain (ECD) of VSIR is an ECD of human VSIR protein, such as wherein the ECD of a human VSIR protein is an amino acid sequence selected from the group consisting of: SEQ ID NO: 380 and SEQ ID NO: 381 (preferably, SEQ ID NO: 381), or an amino acid sequence having at least 80%, 85%, 90%, 92%, 95% or 97% sequence identity (preferably, at least 92% or 95% sequence identity) to these sequences, and/or having no more than two, four, six or eight, for example no more than one, two or three, such as no more than one, amino acid substitutions, insertions or deletions compared to these sequences.

The term “interacting protein”, in the context of IGSF11 (VSIG3) will be art-recognised, but further includes any poly(peptide) that is detectable as binding to IGSF11 (such as, substantially or appreciably binds to IGSF11) or, and in particular, to a domain of IGSF11 such as to an IgC2 domain of IGSF11 (or to an IgV domain of IGSF11). Methods to detect such binding include in-vitro and in-vivo technologies, and which for example, detect the binding between such an interacting protein and IGSF11 that may occur away from a cellular context, or within a cellular context. For example, methods such as enzyme-linked immunosorbent assay (ELISA), surface plasmon resonance (SPR) or bio-layer interferometry (BLI) (eg, analogues to those described in the Examples herein) can be considered “in-vitro” methods to detect binding between a protein and IGSF11 (or domain thereof), and hence enable the person of ordinary skill to identify such polypeptide as an “interacting protein” of IGSF11 (or domain thereof). The examples herein (and the prior art) demonstrate that VSIR (VISTA) is detectable as binding to IGSF11, and hence is one example of an interaction protein of IGSF11. Other methods such as yeast two-hybrid, cell-binding and co-immunoprecipitation can be considered “in-vivo” methods to detect binding between a protein and IGSF11 (or domain thereof), and hence enable the person of ordinary skill to also identify such polypeptide as an “interacting protein” of IGSF11 (or domain thereof). In certain embodiments of any of the (applicable) aspects of the invention, the interacting protein of IGSF11 is an endogenous binding (protein) partner of IGSF11, for example an endogenous IGSF11 ligand or receptor. An “endogenous” binding partner (or receptor/ligand) of IGSF11 (or a domain thereof, such as an IgC2 or IgV domain of IGSF11), is a molecule (typically a polypeptide/protein) that interacts with IGSF11 or, and in particular, with a domain of IGSF11 such as with the IgC2 domain of IGSF11 (or with the IgV domain of IGSF11) I the context of natural physiology or molecule processes of the organism or cell(s). Such physiology or molecule processes may be when such organism or cell(s) has (have) a heathy status, or in other embodiments or it may be when such organism or cell(s) has (have) a diseased status. In other certain embodiments of any of the (applicable) aspects of the invention, the interacting protein of IGSF11 is a biochemical binding (protein) partner, ie one that binds to IGSF11 in a biochemical assay, such as in an ELISA, SPR or BLI.

In one particular embodiment of any of the (applicable) aspects of the invention, the interacting protein is VSIR (VISTA), and in particular the ECD (or portions thereof) of VSIR. In another particular embodiment of any of the (applicable) aspects of the invention, the interacting protein is VSIG8, and in particular the ECD (or portions thereof) of VSIG8.

In a further embodiment, the interacting protein is a protein (eg, an immunoglobulin-like protein other than IGSF11), that is expressed by/on another cell than the one expressing the IGSF11, such as expressed by/on a T cell. Interactions between such protein and IGSF11 would be considered a “trans-interaction”, or an “intercellular” interaction. In an alternative further embodiment, the interacting protein is a protein (eg, an immunoglobulin-like protein that is expressed by/on the same cell as the IGSF11, such as expressed on a tumour cell. Interactions between such protein and IGSF11 would be considered a “cis-interaction”, or an “intracellular” interaction. Examples of such cis-interactions (and hence further examples of interacting protein) include homodimerization between IGSF11 molecules; hence, in one embodiment, the interaction protein of IGSF11, or domain thereof, is another molecule of IGSF11 (eg to form an IGSF11-IGSF11 dimer, or higher homo-multimer). Cis-interactions also include heterodimerisation, such as between IGSF11 and VISIG8. In other embodiments of cis-interactions the interacting protein can be a co-receptor of IGSF11. In another embodiment, the interacting protein of IGSF11 can be a junctional protein, such as a gap junction protein. In yet another embodiment, the interacting protein can be a protein involved in formation, regulation and/or maintenance of an immune synapse, and/or a protein involved in immune synaptic transmission and/or plasticity, in each case such as between and immune cells and a tumour cell (eg a tumour cell that expresses IGSF11).

Modulators of IGSF11 Expression, Function, Activity and/or Stability

In particular embodiments of such aspect, the ABP is a modulator of the expression, function, activity and/or stability of IGSF11 or of an IgC2 domain of IGSF11 (or, in an alternative aspect, an IgV domain of IGSF11), or the variant of IGSF11 (or such domain), such as wherein the ABP inhibits the expression, function, activity and/or stability of IGSF11 or of such domain, or the variant of IGSF11 (or the variant of such domain), or in particular where the ABP is an inhibitor of the function and/or activity of said IGSF11 or of such domain, or the variant of IGSF11 (or the variant of such domain). In one of such embodiments, an ABP of the invention is an inhibitor of the interaction between IGSF11, or the variant of IGSF11, to its interacting protein (for example, to its endogenous binding partner such as an endogenous receptor or ligand), such as to VSIR, or a variant of VSIR. In one particular embodiment, an ABP of the invention is capable of inhibiting (eg, inhibits or is an inhibitor of) the binding of the interacting protein (eg, VSIR (VISTA) protein or a variant thereof) to IGSF11 protein or a variant thereof such as the interacting protein to the IgC2 domain of IGSF11 protein (or to the IgV domain of IGSF11 protein) or a variant of such domain. In another particular embodiment, an ABP of the invention is capable of inhibiting (eg, inhibits or is an inhibitor of) the binding of any of the other proteins described where herein as being an interacting protein to IGSF11 protein or a variant thereof, for example such interacting protein to the IgC2 domain of IGSF11 protein (or to the IgV domain of IGSF11 protein) or a variant of such domain. Accordingly, ABPs of the invention can be “modulators”.

The term “modulator” as used herein, refers to a molecule that changes, modifies or alters one or more characteristics, properties and/or abilities of another molecule or, for example, that changes, modifies or alters an immune response (“immunomodulators”), such as a cell-mediated immune response. For example, a modulator (eg, an inhibiting or antagonistic modulator) can impair or interfere with, or cause a decrease in the magnitude of, expression, function, activity and/or stability, such as a certain activity or function, of a molecule compared to the magnitude of such characteristic, property or ability observed in the absence of the modulator. In an alternative example, a modulator (eg, an activating or agonistic modulator) can enhance or promote, or cause an increase in the magnitude of, expression, function, activity and/or stability, such as a certain activity or function, of a molecule compared to the magnitude of such characteristic, property or ability observed in the absence of the modulator. Certain exemplary characteristics, properties or abilities of a molecule include, but are not limited to, expression, function, activity and/or stability, such as binding ability or affinity, enzymatic activity, and signal transduction; for example, any of the functions or activities of IGSF11 described herein.

Modulatory molecules (in particular, modulatory ABPs) can act as “inhibitors” (“antagonists”) against a receptor such as IGSF11, such as by impairing (e.g. blocking) ligand engagement to such receptor, eg by inhibiting the interaction between IGSF11 (or a domain thereof, such as an IgC2 or IgV domain of IGSF11) and an interacting protein (eg, VSIR) or an endogenous binding partner such as any of those described elsewhere herein. Alternatively, modulatory molecules (in particular, modulatory ABPs) can act as “activators” (“agonists”) for a receptor such as IGSF11, such as by enhancing or promoting function and/or activity of such receptor, for example by triggering the receptor's signalling pathway, such as by mimicking the binding of the endogenous ligand for such receptor.

As used herein, the terms “modulator of IGSF11 expression” and the like (such as an “inhibitor [or antagonist] of IGSF11 expression” and the like) shall relate to any molecule (eg any of the herein disclosed ABPs) which has an effect (such as an antagonistic activity) toward the expression of an IGSF11 protein, that is it alters (e.g. impairs, suppresses, reduces and/or lowers) the expression of an IGSF11 protein (or a domain thereof, such as an IgC2 or IgV domain of IGSF11) such as may be determined by measuring an amount (or change in an amount) of IGSF11 protein or IGSF11 mRNA. A modulator that is an activator or agonist will, typically have the corresponding but inverse effect (to that of an inhibitor or antagonist) on IGSF11 expression, eg that such a modulator enhances, promotes, increases and/or raises IGSF11 expression. The term “expression” means in this context the cellular process of transcribing a gene into an mRNA and the following translation of the mRNA into a protein. “Gene expression” therefore may thus refer only to the generation of mRNA, irrespectively from the fate of the so produced mRNA, or alternatively/additionally to the translation of the expressed mRNA into a protein. The term “protein expression” on the other hand may refer to the complete cellular process of synthesis of proteins. The terms “modulator of expression of a [orthologue][paralogue][variant] of IGSF11 [or domain thereof]” and the like, shall have the corresponding meaning with respect to any such variant of IGSF11 (or variant of such domain).

The terms “modulator of IGSF11 [or domain thereof] stability” and the like (such as an “inhibitor [or antagonist] of IGSF11 (or a domain thereof) stability” and the like) shall refer to any molecule (eg any of the herein disclosed ABPs) which has an effect (such as a negative activity) towards the stability of an IGSF11 protein (or a domain thereof, such as an IgC2 or IgV domain of IGSF11). The term, in context of the present disclosure, shall be understood in its broadest sense. Such modulators are included by the term, which, for example, interfere with and reduce the IGSF11 protein half-live or interfere with and disturb IGSF11 protein (or a domain thereof, such as an IgC2 or IgV domain of IGSF11) folding or protein presentation on the surface of the cell. In one preferred example, an inhibiting modulator of the invention, such as an ABP, may induce internalisation, and optionally degradation, of IGSF11 protein from the surface of the cell. Such internalisation of IGSF11 protein may be detected and/or measured by methods analogous to those describe in Example D herein. A modulatory that is an activator or agonist will, typically have the corresponding but inverse effect (to that of an inhibitor or antagonist) on IGSF11 stability, eg that such a modulator enhances, promotes, increases and/or raises IGSF11 (or a domain thereof) stability. The terms “modulator of stability of a [orthologue][paralogue][variant] of IGSF11 [or domain thereof]” and the like, shall have the corresponding meaning with respect to any such variant of IGSF11 (or variant of such domain).

The terms a “modulator of IGSF11 (or a domain thereof) function [or activity]” and the like (such as an “inhibitor [or antagonist] of IGSF11 (or a domain thereof) function [or activity]” and the like) shall refer to any molecule (eg any of the herein disclosed ABPs) that alters, such as impairs (e.g., induces a decrease or reduction in) the efficiency, effectiveness, amount or rate of one or more activities of IGSF11 (or a domain thereof, such as an IgC2 or an IgV domain of IGSF11) (for example, by impairing the expression and/or stability of IGSF11 protein or a domain thereof), such as one or more of those activities described herein, for example, the activity of IGSF11 (or a domain thereof) as a modulator of T-cell activation and/or viability. In one embodiment, such a modulating ABP may impair binding of one or more of the endogenous binding partners of IGSF11 protein. For example, such a modulator may impair the interaction between IGSF11 protein and VSIR protein (eg, such a modulator may reduce, inhibit or block the binding between IGSF11 protein (or a domain thereof, such as an IgC2 or IgV domain of IGSF11) and an interacting protein such as any of those described elsewhere herein (eg, VSIR protein). A modulator that is an activator or agonist will, typically have the corresponding but inverse effect (to that of an inhibitor or antagonist) on IGSF11 function and/or activity, eg that such a modulator enhances, promotes, increases and/or raises IGSF11 function and/or activity. For example, such a modulator may promote or increase the function or activity of IGSF11 receptor, for example by triggering the signalling pathway of IGSF11. The terms “modulator of function of a [orthologue][paralogue][variant] of IGSF11 (or a domain thereof)” and the like, shall have the corresponding meaning with respect to any such variant of IGSF11 (or variant of such domain).

A particular embodiment of a modulator of IGSF11 (or a domain thereof, such as an IgC2 or IgV domain of IGSF11) is an “inhibitor of IGSF11 (or domain thereof)” (or “IGSF11 inhibitor”, “IgC2 domain of IGSF11 inhibitor” or “IgV domain of IGSF11 inhibitor”), which meaning includes any moiety that inhibits IGSF11 (or a domain of IGSF11, such as an IgC2 or IgV domain of IGSF11), which can mean inhibition of the expression (eg the amount), function, activity and/or stability of IGSF11 (or such domain), especially of mRNA and/or protein of IGSF11 (or domain thereof). In one particular of such embodiments, an inhibitor of IGSF11 (or domain thereof) can reduce the function (and/or activity) of IGSF11 protein, (or domain thereof) and in another of such embodiments, an inhibitor of IGSF11 can reduce the expression of IGSF11 mRNA and/or protein.

Such an IGSF11 inhibiting moiety, or IGSF11 domain inhibiting moiety, can act directly, for example, by binding to IGSF11 or a domain thereof, such as an IgC2 or IgV domain of IGSF11) and decreasing the amount or rate of one or more of the properties of IGSF11 (or such domain) such as its expression, function, activity and/or stability, in particular by inhibiting (eg blocking) its interaction with an interacting protein (eg, VSIR) and/or to increase the sensitivity of a tumour cell expressing IGSF11 to a cell-mediated immune response. A IGSF11 inhibitor, or IGSF11 domain inhibitor, may also decrease the amount or rate of IGSF11 function or activity by impairing its expression or stability, for example, by binding to IGSF11 protein (or a domain of IGSF11 protein) or mRNA and modifying it, such as by removal or addition of a moiety, or altering its three-dimensional conformation; and by binding to IGSF11 protein (or a domain of IGSF11 protein) or mRNA and reducing its stability or conformational integrity. A IGSF11 (or IGSF11 domain) inhibitor may, alternatively, act indirectly, for example, by binding to a regulatory molecule or gene region to modulate such regulatory protein or gene region function and hence consequentially affect a decrease in the amount or rate of IGSF11 expression (eg amount), function/activity and/or stability, in particular by impairing one or more activity of IGSF11 protein (or a domain of IGSF11 protein) or mRNA (such as by changing the amount or rate of expression and/or stability of IGSF11 protein or mRNA). Thus, an IGSF11 (or IGSF11 domain) inhibitor can act by any mechanism(s) that impair, such as result in a decrease in, the amount or rate of IGSF11 expression (eg amount), function/activity and/or stability. Non-limiting examples of IGSF11 (or IGSF11 domain) inhibitors that act directly on IGSF11 (or an IGSF11 domain) include: (i) siRNA or shRNA molecules that bind to and reduce expression of IGSF11 mRNA; and (ii) ABPs that bind to (eg an EC domain, an IgC2 domain or an IgV domain) of IGSF11 protein and reduce the ability of IGSF11 protein (or such domain) to interact with (eg bind to) an interacting protein (eg, VSIR protein) such as an endogenous binding partner to IGSF11 protein. Non-limiting examples of IGSF11 (or IGSF11 domain) inhibitors that act indirectly on IGSF11 (or an IGSF11 domain) include siRNA or shRNA molecules that bind to and reduce expression of mRNA or a gene that enhances the expression or activity of IGSF11, consequential reducing the amount (and hence activity) of IGSF11 protein (or such domain).

General and specific examples of inhibitors of IGSF11 or inhibitors of a domain thereof, such as an inhibitor of an IgC2 or IgV domain of IGSF11 (including those that are ABPs of the present invention) are described elsewhere herein, including those as may be characterised by the applicable functional and/or structural features set out herein.

Accordingly, in particular embodiments of the present invention, an ABP of the invention is one that is capable of specifically binding to (eg which specifically binds to) a C2-type immunoglobulin-like (IgC2) domain of IGSF11 (VSIG3) (or, in another aspect, specifically binds to a V-type immunoglobulin-like (IgV) domain of IGSF11 (VSIG3)), as well as, optionally, being capable of inhibiting (eg reducing or blocking) the interaction between IGSF11 (VSIG3) protein (or a variant thereof, such as one described above) and an interacting protein, such as VSIR (VISTA) protein (or a variant thereof, such as any of those described elsewhere herein). In particular embodiments, such an ABP is able to inhibit (eg inhibits) the binding of the interacting protein (eg VSIR (VISTA) protein, or a variant thereof, such as one described above) to IGSF11 (VSIG3) protein (or a variant thereof, such as one described above).

Methodologies to determine the interaction (eg binding) between an IgC2 domain of (or an IgV domain of) IGSF11 (VSIG3) and the interacting protein (eg, VSIR (VISTA) protein) (or between variants thereof) are known to the person of ordinary skill, and include ELISA assays (such as described in the examples below), and technologies such as inter alia: flow cytometry, surface plasmon resonance, surface acoustic waves and microscale thermophoresis. Such determination methodologies can be used (or adapted) to not only detect the presence of such interaction/binding, but also to measure (eg quantitatively) the degree of binding between the interacting partners IGSF11 (or a domain thereof, such as an inhibitor of an IgC2 or IgV domain of IGSF11) and an interacting protein (eg VSIR proteins, or an endogenous binding partner) (or variants thereof). Such (quantitative) measurement of interaction (binding) may be determined or measured in the presence of a competing (eg inhibiting) ABP of the invention, and hence the potential of an ABP of the present invention to inhibit (eg block) such interaction can be measured, and eg reported as an IC50.

Such IC50 values may be determined, such as using ELISA methodology (eg, using an assay correspond to, or substantially as, the ELISA described in Comparative Example 5), in the presence of a suitable concentration of the interacting protein such as VSIR protein (or variant thereof) in solution and with surface-bound IGSF11 (or domain thereof). Suitable concentrations of VSIR protein (or variant thereof) include: about 100 pM to about 100 uM VSIR protein (or variant thereof), for example about 0.75 ug/mL to about 20 ug/mL of Fc-VSIR fusion (eg, as described in Comparative Example 5), which corresponds to about 8.2 nM to about 222 nM dimer concentration of Fc-VSIR. Preferred suitable concentrations of VSIR protein (or variant thereof) include between about 20 nM to about 100 nM dimer concentration of Fc-VSIR (eg, as described in Comparative Example 5), such as about 75 nM of such Fc-VSIR.

The IC50 of an (inhibitor/antagonist) modulator (eg, an ABP of the invention) can be determined by examining the effect of increasing concentrations of the inhibitor/antagonist modulator on the function and/or activity being investigated as the biological response (for example, an inhibition of binding of the IGSF11 or domain of IGSF11 (or variant thereof) to the VSIR (or variant thereof), or that results in and/or is measured by enhancement of a cell-mediated immune response and/or an increase in immune cell activity and/or survival, such as may be determined using methodologies correspond to, or substantially as, the those described in Comparative Examples 7 and/or 8), from a maximum such response. Responses are then normalized to the maximum and plotted against the log concentration of inhibitor/antagonist modulator in order to construct a dose-response curve, from which the concentration can be determined that gives 50% inhibition of the maximum biological response.

In certain of such embodiments of the invention, the ABP of the invention (eg one that binds to [one or more epitope(s) displayed by] an IgC2 domain (or IgV domain) of IGSF11, or a paralogue, orthologue or other variant thereof) is capable of inhibiting (eg will inhibit) the binding of VSIR protein or a variant thereof to IGSF11 protein (or such domain or IGSF11) or a variant thereof with an IC50 of 100 nM, 50 nM, or preferably 20 nM or less, such as 15 nM or less, 10 nM or less, 5 nM or less, 2 nM or less, 1 nM or less, 500 pM or less, 250 pM or less, or 100 pM or less. In particular of such embodiments, an ABP of the invention is capable of inhibiting (eg will inhibit) the binding of VSIR protein or a variant thereof to IGSF11 protein (or such domain or IGSF11) or a variant thereof with an IC50 of 10 nM or less, such as 5 nM or less and preferably 2 nM or less.

In particular of those embodiments where the ABP of the invention inhibits the interaction (eg the binding) between VSIR and IGSF11 proteins (or a domain or IGSF11, such as an IgC2 domain or an IgV domain of IGSF11) (or variants thereof), the VSIR protein is human VSIR protein and/or the IGSF11 protein is human IGSF11 protein. Preferably (such as in a binding assay to determine the IC50 of such ABP) the VSIR protein is human VSIR protein and the IGSF11 protein is human IGSF11 protein, and in other particular embodiments, the variant of the VSIR protein comprises an ECD of VSIR protein, preferably of a human VSIR protein, and/or the variant of the IGSF11 protein comprises an IgC2 domain of (or an IgV domain of) IGSF11 protein, preferably of a human IGSF11 protein, such as wherein the variant of the VSIR protein comprises an ECD of human VSIR protein, and the variant of the IGSF11 protein comprises an IgC2 domain of (or an IgV domain of) of human IGSF11 protein. In particular of such embodiments, an ABP of the invention is capable of inhibiting (eg inhibits) the interaction between: (i) an IGSF11 protein variant that is the ECD of human IGSF11 protein (optionally his tagged for purification), such as described in Comparative Example 5; and (ii) a VSIR protein variant that is human VSIR-Fc (human IgG1), such as obtainable from R&D Systems (Cat #7126-B7), in particular where such inhibition of the interaction can be detected in an ELISA assay using such proteins, such as an ELISA assay corresponding to, or substantially as, the ELISA described in Comparative Example 5). In other particular of such embodiments, an ABP of the invention is capable of inhibiting (eg inhibits) the interaction between: (i) an IgC2 domain of a IGSF11 protein variant (optionally his tagged for purification), such as described in Example 15; and (ii) a VSIR protein variant that is human VSIR-Fc (human IgG1), such as obtainable from R&D Systems (Cat #7126-B7) or as described in Example 15, in particular where such inhibition of the interaction can be detected in an ELISA or SPR assay using such proteins, such as an ELISA or SPR assay corresponding to, or substantially as, the ELISA or SPR assay described in Example 15).

In other embodiments, a modulator of the invention (eg, an ABP that binds to an IgC2 domain of (or an IgV domain of) IGSF11) that is an inhibitor or antagonist may instead or also:
inhibit, impair, reduce or reverse IGSF11-mediated inhibition of a cell-mediated immune response (eg in an in-vitro assay or in a subject, such as one in need thereof); and/or
inhibit, impair, reduce or reverse IGSF11-mediated inhibition of humoral immunity (eg in an in-vitro assay or in a subject, such as one in need thereof).

The term “cell-mediated immune response” as used herein, may include, but is not limited to, a response in a host organism involving, utilising, and/or promoting any one or combinations of T cell maturation, proliferation, activation, migration, infiltration and/or differentiation, and/or the activation/modulation/migration/infiltration of a macrophage, a natural killer cell, a T lymphocyte (or T cell), a helper T lymphocyte, a memory T lymphocyte, a suppressor T lymphocyte, a regulator T lymphocyte, and/or a cytotoxic T lymphocyte (CTL), and/or the production, release, and/or effect of one or more cell-secretable or cell-secreted factor such as a cytokine or autocoid (in particular a pro-inflammatory cytokine), and/or one or more components of any of such processes (such as a cytokine or autocoid, particular a pro-inflammatory cytokine). The term “cell-mediated immune response,” as used herein, may include a cellular response involving a genetically engineered, in-vitro cultured, autologous, heterologous, modified, and/or transferred T lymphocyte, or it may include a cell-secretable or cell-secreted factor (such as a cytokine or autocoid, in particular a pro-inflammatory cytokine) produced by genetic engineering. A cell-mediated immune response is preferably not a humoral immune response, such as an immune response involving the release of antibodies. In certain embodiments, in particular when the proliferative disorder is a cancer or tumour, the cell-mediated immune response is an anti-tumour cell-mediated immune response. For example, one that leads to a reduction in tumour (cell) growth, such as a cytotoxic cell-mediated immune response (such as a cytotoxic T cell exposure) that kills cells of the cancer or tumour.

In certain embodiments, the cell mediating the cell-mediated immune response may be mediated by a cell, such as an immune cell, capable of secreting (eg secreting) pro-inflammatory cytokine, such as one selected from the group consisting of: interleukin-1 (IL-1), IL-2, IL-12, IL-17 and IL-18, tumour necrosis factor (TNF) [alpha], interferon gamma (IFN-gamma), and granulocyte-macrophage colony stimulating factor.

In certain embodiments, the cell-mediated immune response can be mediated by a pro-inflammatory cytokine-secreting cell, such as a lymphocyte (eg a T cell), in particular a cytotoxic T lymphocyte (CTL).

In particular embodiments, the cell-mediated immune response may induce killing of cells associated or involved with a disease, disorder or condition, such as a proliferative disorder (eg a cancer).

The term “humoral immunity” (or “humoral immune response”) will also be readily understood by the person of ordinary skill, and includes an aspect of an immune response that is mediated by macromolecules found in extracellular fluids such as secreted antibodies, complement proteins, and certain antimicrobial peptides. Humoral immunity is so named because it involves substances found in the humors, or body fluids. Its aspects involving antibodies can be termed antibody-mediated immunity.

As used herein, a “subject” includes all mammals, including without limitation humans, but also non-human primates such as cynomolgus monkeys. It also includes dogs, cats, horses, sheep, goats, cows, rabbits, pigs and rodents (such as mice and rats). It will be appreciated that a particularly preferred subject according to the invention is a human subject, such as a human suffering from (or at risk of suffering from) a disorder, disease or condition, for example a human patient.

In further other embodiments, a modulator of the invention (eg, an ABP that binds to an IgC2 domain of (or an IgV domain of) IGSF11) that is an inhibitor or antagonist may instead or also:
increase B cell proliferation or B cell responses including but not limited to antigen-specific antibody responses (eg in an in-vitro assay or in a subject, such as one in need thereof);
promote humoral immune responses elicited against an antigen or cell or therapeutic antibody (eg in an in-vitro assay or in a subject, such as one in need thereof);
promote humoral immune responses elicited by a therapeutic or prophylactic vaccine (eg in an in-vitro assay or in a subject, such as one in need thereof);
mediate any one or combination of at least one of the following effects: (i) increases immune response, (ii) increases T cell activation, (iii) increases cytotoxic T cell activity, (iv) increases NK cell activity, (v) alleviates T-cell suppression, (vi) increases pro-inflammatory cytokine secretion, (vii) increases IL-2 secretion; (viii) increases interferon-gamma production, (ix) increases Th1 response, (x) decreases Th2 response, (xi) decreases or eliminates cell number and/or activity of at least one of regulatory T cells (Tregs), myeloid derived suppressor cells (MDSCs), iMCs, mesenchymal stromal cells, TIE2-expressing monocytes, (xii) reduces regulatory cell activity, and/or the activity of one or more of myeloid derived suppressor cells (MDSCs), iMCs, mesenchymal stromal cells, TIE2-expressing monocytes, (xiii) reduces or decreases (eg, the number of) or eliminates M2 macrophages, (xiv) reduces M2 macrophage pro-tumorigenic activity, (xv) decreases or eliminates N2 neutrophils, (xvi) reduces N2 neutrophils pro-tumorigenic activity, (xvii) reduces inhibition of T cell activation, (xviii) reduces inhibition of CTL activation, (xix) reduces inhibition of NK cell activation, (xx) reverses T cell exhaustion, (xxi) increases T cell response, (xxii) increases activity of cytotoxic cells, (xxiii) stimulates antigen-specific memory responses, (xxiv) elicits apoptosis or lysis of cancer cells, (xxv) stimulates cytotoxic or cytostatic effect on cancer cells, (xxvi) induces direct killing of cancer cells, (xxvii) increases Th17 activity and/or (xxviii) induces complement dependent cytotoxicity and/or antibody dependent cell-mediated cytotoxicity; (eg in an in-vitro assay or in a subject, such as one in need thereof) with the optional proviso that said modulator may elicit an opposite effect to one or more of (i)-(xxviii).

In further other embodiments, a modulator of the invention (eg, an ABP that binds to an IgC2 domain of (or an IgV domain of) IGSF11) may modify the microenvironment of a tumour. For example, such a modulator of the invention may modulate the number and/or type of immune cells present in the tumour, for example: (i) such a modulator that is an inhibitor or antagonist may instead or also reduce the number of intra-tumoural myeloid-derived suppressor cells (MDSCs), in particular granulocytic MDSCs (gMDSCs) or monocytic MDSCs (mMDSCs), and/or increase the number of intra-tumoural CTLs; and (ii) such a modulator that is an activator or agonist may instead or also increase the number of intra-tumoural myeloid-derived suppressor cells (MDSCs), in particular granulocytic MDSCs (gMDSCs) or monocytic MDSCs (mMDSCs), and/or reduce the number of intra-tumoural CTLs. The term microenvironment of a tumour (or “tumour microenvironment” (TME)) is art-recognised, and includes the meaning being the environment around a tumour, including the surrounding blood vessels, immune cells (such as T cells and myeloid-derived suppressor cells), fibroblasts, signalling molecules and the extracellular matrix. The tumour and the surrounding microenvironment are closely related and interact constantly. Tumours can influence the microenvironment by releasing extracellular signals, promoting tumour angiogenesis and inducing peripheral immune tolerance, while the immune cells (eg CTLs) in the TME can affect the growth and evolution of cancerous cells.

In alternative embodiments, a modulator of the invention (eg, an ABP that binds to an IgC2 domain of (or an IgV domain of) IGSF11) that is an activator or agonist may instead or also:
enhance, raise, promote or increase IGSF11-mediated inhibition of a cell-mediated immune response (eg in an in-vitro assay or in a subject, such as one in need thereof); and/or
enhance, raise, promote or increase IGSF11-mediated inhibition of humoral immunity (eg in an in-vitro assay or in a subject, such as one in need thereof)).

In further alternative embodiments, a modulator of the invention (eg, an ABP that binds to an IgC2 domain of (or an IgV domain of) IGSF11) that is an activator or agonist may instead or also:
suppress B cell proliferation or B cell responses including but not limited to antigen-specific antibody responses i (eg in an in-vitro assay or in a subject, such as one in need thereof); and/or
mediate any one or combination of at least one of the following effects: (i) decreases immune response, (ii) decreases T cell activation, (iii) decreases cytotoxic T ceil activity, (iv) decreases natural killer (NK) cell activity, (v) decreases T-cell activity, (vi) decreases pro-inflammatory cytokine secretion, (vii) decreases IL-2 secretion; (viii) decreases interferon-gamma production, (ix) decreases Th1 response, (x) decreases Th2 response, (xi) increases cell number and/or activity of regulatory T cells, (xii) increases regulatory cell activity and/or one or more of myeloid derived suppressor cells (MDSCs), iMCs, mesenchymal stromal cells, TIE2-expressing monocytes, (xiii) increases regulatory cell activity and/or the activity of one or more of myeloid derived suppressor cells (MDSCs), iMCs, mesenchymal stromal cells, TIE2-expressing monocytes, (xiii) increases M2 macrophages, (xiv) increases M2 macrophage activity, (xv) increases N2 neutrophils, (xvi) increases N2 neutrophils activity, (xvii) increases inhibition of T cell activation, (xviii) increases inhibition of CTL activation, (xix) increases inhibition of NK cell activation, (xx) increases T cell exhaustion, (xxi) decreases T cell response, (xxii) decreases activity of cytotoxic cells, (xxiii) reduces antigen-specific memory responses, (xxiv) inhibits apoptosis or lysis of cells, (xxv) decreases cytotoxic or cytostatic effect on cells, (xxvi) reduces direct killing of cells, (xxvii) decreases Th17 activity, and/or (xxviii) reduces complement dependent cytotoxicity and/or antibody dependent cell-mediated cytotoxicity; (eg in an in-vitro assay or in a subject, such as one in need thereof) with the optional proviso that said modulator may elicit an opposite effect to one or more of (i)-(xxviii).

ABPs of the Invention Comprising One or More Complementarity Determining Regions

In particular embodiments, a compound of the invention is an ABP that specifically binds to an IgC2 domain (or to an IgV domain) of immunoglobulin superfamily member 11 (IGSF11, or VSIG3), or of a variant of such domain of IGSF11. Such an ABP is one example of an “IGSF11/domain binder” (as such term is used herein)

In particular embodiments, an ABP of the invention can preferentially comprise at least one complementarity determining region (CDR), such as one from an antibody (in particular from a human antibody), and in particular embodiments the ABP can comprise a CDR having an amino acid sequence with at least 80%, 85%, 90% or 95% sequence identity to (preferably, at least 90% sequence identity to), or having no more than eight, seven, six, five or four (eg, for L-CDR3), such as having no more than three or two, preferably no more than one amino acid substitution(s), deletion(s) or insertion(s) (in particular, substitution(s)) compared to, a CDR sequence set forth in Table 13.1A herein.

The term “complementarity determining region” (or “CDR” or “hypervariable region”), as used herein, refers broadly to one or more of the hyper-variable or complementarity determining regions (CDRs) found in the variable regions of light or heavy chains of an antibody. See, for example: “IMGT”, Lefranc et al, 20003, Dev Comp Immunol 27:55; Honegger & Pluckthun, 2001, J Mol Biol 309:657, Abhinandan & Martin, 2008, Mol Immunol 45:3832, Kabat, et al. (1987): Sequences of Proteins of Immunological Interest National Institutes of Health, Bethesda, Md. These expressions include the hypervariable regions as defined by Kabat et al (1983) Sequences of Proteins of Immunological Interest, US Dept of Health and Human Services, or the hypervariable loops in 3-dimensional structures of antibodies (Chothia and Lesk, 1987; J Mol Biol 196:901). The CDRs in each chain are held in close proximity by framework regions and, with the CDRs from the other chain, contribute to the formation of the antigen-binding site. Within the CDRs there are select amino acids that have been described as the selectivity determining regions (SDRs) which represent the critical contact residues used by the CDR in the antibody-antigen interaction. (Kashmiri, 2005; Methods 36:25).

As described above, in particular embodiments of the invention, an ABP can comprise at least one complementarity determining region (CDR). In certain of such embodiments, an ABP of the invention comprises at least one complementarity determining region 3 (CDR3), such as one having an amino acid sequence with at least 80%, 85%, 90% or 95% (preferably at least 90%) sequence identity to, or having no more than eight, seven, six, five or four (eg, for L-CDR3), such as having no more than three or two (eg, for H-CDR3), preferably no more than one amino acid substitution(s), deletion(s) or insertion(s) (in particular, substitution(s)) compared to, a sequence selected from those heavy and light chain CDR3 sequences shown in Table 13.1A (eg, a sequence selected from the list consisting of SEQ ID Nos: 393, 397, 403, 407, 413, 417, 423, 427, 433, 437, 443, 447, 453, 457, 463, 467, 473, 477, 483, 487, 493, 497, 503, 507, 513, 517, 523, 527, 533, 537, 543, 547, 553, 557, 563, 567, 573, 577, 583, 587, 593, 597, 603, 607, 613, 617, 623, 627, 633, 637, 643, 647, 653, 657, 663, 667, 673, and 677; and/or in particular eg an amino acid sequence of a CDR3 as shown in Table 13.1A for the corresponding heavy chain or light chain CDR3 of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007; such as a sequence selected from SEQ ID Nos.: 403, 407, 413, 417, 423, 427, 433, 437, 443, 447, 483, 487, 493, 497, 513, 517, 523, 527, 533, 537, 563, 567, 593, 597, 603, 607, 613 and 617 (or, in the other aspect, such as a sequence selected from SEQ ID Nos: 393, 397, 453, 457, 463, 467, 473, 477, 543, 547, 553, 557, 623, 627, 633, 637, 643 and 647)).

As described above, in particular further embodiments of the invention, a further ABP can comprise at least one complementarity determining region (CDR). In certain of such embodiments, an ABP of the invention comprises at least one complementarity determining region 3 (CDR3), such as one having an amino acid sequence with at least 80%, 85%, 90% or 95% (preferably at least 90%) sequence identity to, or having no more than eight, seven, six, five or four (eg, for L-CDR3), such as having no more than three or two (eg, for H-CDR3), preferably no more than one amino acid substitution(s), deletion(s) or insertion(s) (in particular, substitution(s)) compared to, a sequence selected from those heavy and light chain CDR3 sequences shown in Table 13.3 (eg, a sequence selected from the list consisting of SEQ ID Nos: 683, 687, 693, 697, 703, 707, 713, 717, 723, 727, 733, 737, 743, 747, 753, 757, 763, 767, 773, 777, 783, 787, 793, 797, 803, 807, 813, 817, 823, 827, 833, 837, 843, 847, 853, 857, 863, 867, 873, 877, 883, 887, 893, 897, 903, 907, 913, 917, 923, 927, 933, 937, 943, 947, 953, 957, 963, 967, 973, 977, 983, 987, 993, 997, 1003, 1007, 1013, 1017, 1023, 1027, 1033, 1037, 1043, 1047, 1053, 1057, 1063, and 1067; and/or in particular eg an amino acid sequence of a CDR3 as shown in Table 13.3 for the corresponding heavy chain or light chain CDR3 of an antibody selected from any one of the antibodies of the group consisting of: D-101, D-102, D-103, D-104, D-105, D-106, D-107, D-108, D-109, D-110, D-111, D-112, D-113, D-114, D-115, D-116, D-201, D-202, D-203, D-204, D-205, D-206, D-207, D-208, D-209, D-210, D-211, D-212, D-213, D-214, D-215, D-216, D-217, D-218, D-219, D-220, D-221, D-222, and D-223, preferably D-114, D-115, or D-116 (eg, D-114), and/or selected from any one of the antibodies of the group consisting of: D-222 or D-223, preferably D-222; such as a sequence selected from SEQ ID Nos.: 813, 817, 823, 827, 833, 837, 1053, 1057, 1063, and 1067).

An ABP of the invention may, alternatively or as well as a CDR3 sequence, comprise at least one CDR1, and/or at least one CDR2 (such as one from an antibody, in particular from a human antibody). Preferably, and ABP of the invention comprises at least one such CDR3, as well as at least one such CDR1 and at least one such CDR2, more preferably where each of such CDRs having an amino acid sequence with at least 80%, 85%, 90% or 95% (preferably at least 90%) sequence identity to, or having no more than five or four (eg, for L-CDR1), such as having no more than three or two, preferably no more than one amino acid substitution(s), deletion(s) or insertion(s) (in particular, substitution(s)) compared to, a sequence selected from the corresponding (heavy and light chain) CDR1, CDR2 and CDR3 sequences shown in (i) Table 13.1A (eg compared to an amino acid sequence of a CDR1, CDR2 and/or CDR3 sequence of the corresponding (heavy and light chain) CDR1, CDR2 and CDR3 sequences as shown in Table 13.1A for an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007; or (ii) Table 13.3 (eg compared to an amino acid sequence of a CDR1, CDR2 and/or CDR3 sequence of the corresponding (heavy and light chain) CDR1, CDR2 and CDR3 sequences as shown in Table 13.3 D-101 to D-116, or D-201 to D-223, preferably D-114, D-115, or D-116 (eg, D-114), and/or selected from any one of the antibodies of the group consisting of: D-222 or D-223, preferably D-222).

In particular embodiments, an ABP of the invention can be an antibody or an antigen binding fragment thereof.

As used herein, the term “antibody” may be understood in the broadest sense as any immunoglobulin (Ig) that enables binding to its epitope. An antibody as such is a species of an ABP. Full length “antibodies” or “immunoglobulins” are generally heterotetrameric glycoproteins of about 150 kDa, composed of two identical light and two identical heavy chains. Each light chain is linked to a heavy chain by one covalent disulphide bond, while the number of disulphide linkages varies between the heavy chain of different immunoglobulin isotypes. Each heavy and light chain also has regularly spaced intrachain disulphide bridges. Each heavy chain has an amino terminal variable domain (VH) followed by three carboxy terminal constant domains (CH). Each light chain has a variable N-terminal domain (VL) and a single C-terminal constant domain (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to cells or factors, including various cells of the immune system (e.g., effector cells) and the first component (C1q) of the classical complement system. Other forms of antibodies include heavy-chain antibodies, being those which consist only of two heavy chains and lack the two light chains usually found in antibodies. Heavy-chain antibodies include the hcIgG (IgG-like) antibodies of camelids such as dromedaries, camels, llamas and alpacas, and the IgNAR antibodies of cartilaginous fishes (for example sharks). And yet other forms of antibodies include single-domain antibodies (sdAb, called Nanobody by Ablynx, the developer) being an antibody fragment consisting of a single monomeric variable antibody domain. Single-domain antibodies are typically produced from heavy-chain antibodies, but may also be derived from conventional antibodies.

Antibodies (or those from which fragments thereof can be isolated) can include, for instance, chimeric, humanized, (fully) human, or hybrid antibodies with dual or multiple antigen or epitope specificities, antibody fragments and antibody sub-fragments, e.g., Fab, Fab′ or F(ab′)2 fragments, single chain antibodies (scFv) and the like (described below), including hybrid fragments of any immunoglobulin or any natural, synthetic or genetically engineered protein that acts like an antibody by binding to a specific antigen to form a complex. VSIR, VSIG8 and IGSF11, and similar types of proteins, are each an immunoglobulin-like protein, and as such each is not (nor its variants) considered—for the purposes of the present invention—an antibody that binds to IGSF11.

In some embodiments of the herein disclosed invention, the ABPs and further ABPs are defined by sequence similarity to CDR and/or variable domain regions of the specific examples of antibodies discovered herein, namely (i) antibodies C-001 to C-029, in particular antibodies C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007; or (ii) antibodies D-101 to D-116, or D-201 to D-223, in particular antibodies D-114, D-115, or D-116 (eg, D-114), and/or in particular antibodies of the group consisting of: D-222 or D-223, preferably D-222. Particularly preferred are such embodiments where compared to the herein disclosed sequence, the corresponding sequence defining the ABP of the invention comprises one or more amino acid substitution(s), deletion(s) or insertion(s) (in particular, substitution(s)); for example: (i) the CDR sequence defining an ABP of the invention may have at least 80%, 85%, 90% or 95% (preferably at least 90%) sequence identity to, or may have no more than five or four, such as may have no more than three or two, preferably no more than one amino acid substitution(s), deletion(s) or insertion(s) (in particular, substitution(s)) compared to, the corresponding CDR sequence disclosed herein; and/or (ii) the variable chain sequence defining an ABP of the invention may have at least 80%, 85%, 90%; or 95% (preferably at least 90%) sequence identity to, or may have no more than fifteen, fourteen, thirteen, twelve or eleven (eg, for variable light chain), such as may have no more than about 20, 18, 16, 14 or 12, or no more than ten, nine, eight, seven, six, five, four, three, two or one, preferably no more than three, two or one amino acid substitution(s), deletion(s) or insertion(s) (in particular, substitution(s)) compared to, the corresponding variable chain sequence disclosed herein, in each case independently, optionally a conservative amino acid substitution. In these embodiments, the following is specifically preferred. A CDR3 sequence (eg, a H-CDR3) in preferred embodiments may vary by no more than one amino acid substitution(s), deletion(s) or insertion(s) (in particular, substitution(s)) compared to a sequence selected from the corresponding (preferably light chain) CDR3 sequences shown in (i) Table 13.1A (in particular, of a CDR3 sequence of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007, and/or is not located at amino acid positions 1, 4 and/or 11 of L-CDR3; and/or is a conservative amino acid substitution; and/or is an amino acid substitution from said CDR3 sequence, most preferably is a substitution from s to t, t to s, s to g, g to s and/or s to a or a to s); or shown in (ii) Table 13.3 (in particular, of a CDR3 sequence of an antibody selected from any one of the antibodies of the group consisting of: D-101 to D-116, and D-201 to D-223, preferably D-114, D-115, or D-116 (eg, D-114), and/or selected from the D-222 or D-223, preferably D-222; and/or is not located at amino acid positions 1, 4 and/or 11 of L-CDR3; and/or is a conservative amino acid substitution; and/or is an amino acid substitution from said CDR3 sequence, most preferably is a substitution from s to t, t to s, s to g, g to s and/or s to a or a to s). Alternatively or additionally, a CDR2 sequence in preferred embodiments may vary by no more than one amino acid substitution(s), deletion(s) or insertion(s) (in particular, substitution(s)) compared to a sequence selected from the corresponding (preferably light chain) CDR2 sequences shown in (i) Table 13.1A (in particular, of a CDR2 sequence of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007, and/or is a conservative amino acid substitution); or shown in (ii) Table 13.3 (in particular, of a CDR2 sequence of an antibody selected from any one of the antibodies of the group consisting of: D-101 to D-116, and D-201 to D-223, preferably D-114, D-115, or D-116 (eg, D-114), and/or selected from the D-222 or D-223, preferably D-222; and/or is a conservative amino acid substitution). Alternatively or additionally, a CDR1 sequence in preferred embodiments may vary by no more than four, preferably no more than three, amino acid substitution(s), deletion(s) or insertion(s) (in particular, substitution(s)) compared to a sequence selected from the corresponding (preferably light chain) CDR1 sequences shown in (i) Table 13.1A (in particular, of a CDR1 sequence of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007, and/or is a conservative amino acid substitution); or shown in (ii) Table 13.3 (in particular, of a CDR1 sequence of an antibody selected from any one of the antibodies of the group consisting of: D-101 to D-116, and D-201 to D-223, preferably D-114, D-115, or D-116 (eg, D-114), and/or selected from the D-222 or D-223, preferably D-222; and/or is a conservative amino acid substitution). Alternatively or additionally, a variable region sequence in preferred embodiments may vary by no more than about 20, 18, 16, 15 or 14, such as not more than about 13 amino acid substitution(s), deletion(s) or insertion(s) (in particular, substitution(s)) (eg, in each case independently, optionally a conservative amino acid substitution) compared to a sequence selected from the corresponding (preferably light chain) variable sequences shown in (i) Table 13.1A (in particular, of a variable region sequence of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007, preferably, wherein independently of the above said for CDR1 to CDR3, no more than about 16, 14 12 or 10, or not more than nine, eight, seven amino acid substitution(s), deletion(s) or insertion(s) (in particular, substitution(s)) located in the variable region framework, and/or in the case of an antibody heavy chain variable region not more than two amino acid substitution(s), deletion(s) or insertion(s) (in particular, substitution(s)) located in the FRI region); or shown in (ii) Table 13.3 (in particular, of a variable region sequence of an antibody selected from any one of the antibodies of the group consisting of: D-101 to D-116, and D-201 to D-223, preferably D-114, D-115, or D-116 (eg, D-114), and/or selected from the D-222 or D-223, preferably D-222, preferably, wherein independently of the above said for CDR1 to CDR3, no more than about 16, 14 12 or 10, or not more than nine, eight, seven amino acid substitution(s), deletion(s) or insertion(s) (in particular, substitution(s)) located in the variable region framework, and/or in the case of an antibody heavy chain variable region not more than two amino acid substitution(s), deletion(s) or insertion(s) (in particular, substitution(s)) located in the FRI region).

Accordingly, in certain embodiments an ABP of the invention can comprise an antibody heavy chain, or an antigen binding fragment thereof, and/or an antibody light chain, or an antigen binding fragment thereof.

In further embodiments, an ABP of the invention can comprise an antibody heavy chain variable region, or an antigen binding fragment thereof, and/or an antibody light chain variable region, or an antigen binding fragment thereof, and in yet further embodiments, an ABP of the invention can comprise an antibody heavy chain variable region CDR1, CDR2, and CDR3, and/or an antibody light chain variable region CDR1, CDR2, and CDR3.

In particular embodiments of the invention, when the ABP comprises an antibody heavy chain sequence and/or an antibody light chain sequence, or an antigen binding fragment thereof; the antibody heavy chain sequence, or the fragment thereof, can comprise a CDR3 having at least 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a CDR3 sequence selected from those heavy chain CDR3 sequences shown in Table 13.1A (eg, a sequence selected from the list consisting of SEQ ID Nos: 393, 403, 413, 423, 433, 443, 453, 463, 473, 483, 493, 503, 513, 523, 533, 543, 553, 563, 573, 583, 593, 603, 613, 623, 633, 643, 653, 663, and 673; and/or in particular eg an amino acid sequence of a heavy chain CDR3 as shown in Table 13.1A for the corresponding heavy chain CDR3 of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007, such as a sequence selected from SEQ ID Nos.: 403, 413, 423, 433, 443, 483, 493, 513, 523, 533, 563, 593, 603 and 613 (or, in the other aspect, such as a sequence selected from SEQ ID Nos: 393, 453, 463, 473, 543, 553, 623, 633 and 643), and/or wherein antibody light chain sequence, or the fragment thereof, can comprise a CDR3 having at least 80%, 85%, 90%; or 95% (preferably at least 90%) sequence identity to, or having no more than eight, seven, six, five or four, such as having no more than three or two, preferably no more than one amino acid substitution(s), deletion(s) or insertion(s) (in particular, substitution(s)) compared to, a CDR3 sequence selected from those light chain CDR3 sequences shown in Table 13.1A (eg, a sequence selected from the list consisting of SEQ ID Nos: 397, 407, 417, 427, 437, 447, 457, 467, 477, 487, 497, 507, 517, 527, 537, 547, 557, 567, 577, 587, 597, 607, 617, 627, 637, 647, 657, 667, and 677; and/or in particular eg an amino acid sequence of a light chain CDR3 as shown in Table 13.1A for the corresponding light chain CDR3 of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007, such as a sequence selected from SEQ ID Nos.: 407, 417, 427, 437, 447, 487, 497, 517, 527, 537, 567, 597, 607 and 617 (or, in the other aspect, such as a sequence selected from SEQ ID Nos: 397, 457, 467, 477, 547, 557, 627, 637 and 647).

In particular further embodiments of the invention, when the further ABP comprises an antibody heavy chain sequence and/or an antibody light chain sequence, or an antigen binding fragment thereof; the antibody heavy chain sequence, or the fragment thereof, can comprise a CDR3 having at least 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a CDR3 sequence selected from those heavy chain CDR3 sequences shown in Table 13.3 (eg, a sequence selected from the list consisting of SEQ ID Nos: 683, 693, 703, 713, 723, 733, 743, 753, 763, 773, 783, 793, 803, 813, 823, 833, 843, 853, 863, 873, 883, 893, 903, 913, 923, 933, 943, 953, 963, 973, 983, 993, 1003, 1013, 1023, 1033, 1043, 1053, and 1063; and/or in particular eg an amino acid sequence of a heavy chain CDR3 as shown in Table 13.3 for the corresponding heavy chain CDR3 of an antibody selected from any one of the antibodies of the group consisting of: D-101 to D-116, and D-201 to D-223, preferably D-114, D-115, or D-116 (eg, D-114), and/or selected from D-222 or D-223, preferably D-222, such as a sequence selected from SEQ ID Nos.: 813, 823, 833, 1053, and 1063; and/or wherein antibody light chain sequence, or the fragment thereof, can comprise a CDR3 having at least 80%, 85%, 90%; or 95% (preferably at least 90%) sequence identity to, or having no more than eight, seven, six, five or four, such as having no more than three or two, preferably no more than one amino acid substitution(s), deletion(s) or insertion(s) (in particular, substitution(s)) compared to, a CDR3 sequence selected from those light chain CDR3 sequences shown in Table 13.3 (eg, a sequence selected from the list consisting of SEQ ID Nos: 687, 697, 707, 717, 727, 737, 747, 757, 767, 777, 787, 797, 807, 817, 827, 837, 847, 857, 867, 877, 887, 897, 907, 917, 927, 937, 947, 957, 967, 977, 987, 997, 1007, 1017, 1027, 1037, 1047, 1057, and 1067; and/or in particular eg an amino acid sequence of a light chain CDR3 as shown in Table 13.3 for the corresponding light chain CDR3 of an antibody selected from any one of the antibodies of the group consisting of: D-101 to D-116, and D-201 to D-223, preferably D-114, D-115, or D-116 (eg, D-114), and/or selected from D-222 or D-223, preferably D-222, such as a sequence selected from SEQ ID Nos.: 817, 827, 837, 1057, and 1067).

In further embodiments of the invention, when the ABP comprises an antibody heavy chain, or an antigen binding fragment thereof, the antibody heavy chain sequence, or the fragment thereof, can further comprise a CDR1 having at least 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a sequence selected from SEQ ID NOs. 391, 401, 411, 421, 431, 441, 451, 461, 471, 481, 491, 501, 511, 521, 531, 541, 551, 561, 571, 581, 591, 601, 611, 621, 631, 641, 651, 661, and 671; (eg a heavy chain CDR1 sequence disclosed in Table 13.1A); and/or in particular eg an amino acid sequence of a heavy chain CDR1 as shown in Table 13.1A for the corresponding heavy chain CDR1 of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007; and/or a CDR2 having at 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a sequence selected from SEQ ID NOs. 392, 402, 412, 422, 432, 442, 452, 462, 472, 482, 492, 502, 512, 522, 532, 542, 552, 562, 572, 582, 592, 602, 612, 622, 632, 642, 652, 662, and 672 (eg a CDR2 sequence disclosed in Table 13.1A); and/or in particular eg an amino acid sequence of a heavy chain CDR2 as shown in Table 13.1A for the corresponding heavy chain CDR2 of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007.

In further embodiments of the invention, when the further ABP comprises an antibody heavy chain, or an antigen binding fragment thereof, the antibody heavy chain sequence, or the fragment thereof, can further comprise a CDR1 having at least 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a sequence selected from SEQ ID NOs. 681, 691, 701, 711, 721, 731, 741, 751, 761, 771, 781, 791, 801, 811, 821, 831, 841, 851, 861, 871, 881, 891, 901, 911, 921, 931, 941, 951, 961, 971, 981, 991, 1001, 1011, 1021, 1031, 1041, 1051, and 1061; (eg a heavy chain CDR1 sequence disclosed in Table 13.3); and/or in particular eg an amino acid sequence of a heavy chain CDR1 as shown in Table 13.3 for the corresponding heavy chain CDR1 of an antibody selected from any one of the antibodies of the group consisting of: D-101 to D-116, and D-201 to D-223, preferably D-114, D-115, or D-116 (eg, D-114), and/or selected from D-222 or D-223, preferably D-222, such as a sequence selected from SEQ ID Nos.: 811, 821, 831, 1051 and 1061; and/or a CDR2 having at 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a sequence selected from SEQ ID NOs. 682, 692, 702, 712, 722, 732, 742, 752, 762, 772, 782, 792, 802, 812, 822, 832, 842, 852, 862, 872, 882, 892, 902, 912, 922, 932, 942, 952, 962, 972, 982, 992, 1002, 1012, 1022, 1032, 1042, 1052, and 1062, (eg a CDR2 sequence disclosed in Table 13.3); and/or in particular eg an amino acid sequence of a heavy chain CDR2 as shown in Table 13.3 for the corresponding heavy chain CDR2 of an antibody selected from any one of the antibodies of the group consisting of D-101 to D-116, and D-201 to D-223, preferably D-114, D-115, or D-116 (eg, D-114), and/or selected from D-222 or D-223, preferably D-222, such as a sequence selected from SEQ ID Nos.: 812, 822, 832, 1052 and 1062.

In yet further embodiments of the present invention, an ABP of the invention comprises an antibody light chain, or an antigen binding fragment thereof, wherein the antibody light chain sequence, or the fragment thereof, further comprises a CDR1 having at least 80%, 85%, 90%; or 95% (preferably at least 90%) sequence identity to, or having no more than five or four (eg, for L-CDR1), such as having no more than three or two, preferably no more than one amino acid substitution(s), deletion(s) or insertion(s) (in particular, substitution(s)) compared to, a sequence selected from SEQ ID NOs. 395, 405, 415, 425, 435, 445, 455, 465, 475, 485, 495, 505, 515, 525, 535, 545, 555, 565, 575, 585, 595, 605, 615, 625, 635, 645, 655, 665, and 675 (eg a light chain CDR1 sequence disclosed in Table 13.1A); and/or in particular compared to eg an amino acid sequence of a light chain CDR1 as shown in Table 13.1A for the corresponding light chain CDR1 of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007; and/or a CDR2 having at least 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a sequence selected from SEQ ID NOs. 396, 406, 416, 426, 436, 446, 456, 466, 476, 486, 496, 506, 516, 526, 536, 546, 556, 566, 576, 586, 596, 606, 616, 626, 636, 646, 656, 666, and 676 (eg a light chain CDR2 sequence disclosed in Table 13.1A); and/or in particular eg compared to an amino acid sequence of a light chain CDR2 as shown in Table 13.1A for the corresponding light chain CDR2 of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007.

In further embodiments of the invention, when the further ABP comprises an antibody light chain, or an antigen binding fragment thereof, the antibody light chain sequence, or the fragment thereof, can further comprise a CDR1 having at least 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a sequence selected from SEQ ID NOs. 685, 695, 705, 715, 725, 735, 745, 755, 765, 775, 785, 795, 805, 815, 825, 835, 845, 855, 865, 875, 885, 895, 905, 915, 925, 935, 945, 955, 965, 975, 985, 995, 1005, 1015, 1025, 1035, 1045, 1055, and 1065; (eg a light chain CDR1 sequence disclosed in Table 13.3); and/or in particular eg an amino acid sequence of a light chain CDR1 as shown in Table 13.3 for the corresponding light chain CDR1 of an antibody selected from any one of the antibodies of the group consisting of: D-101 to D-116, and D-201 to D-223, preferably D-114, D-115, or D-116 (eg, D-114), and/or selected from D-222 or D-223, preferably D-222, such as a sequence selected from SEQ ID Nos.: 815, 825, 835, 1055 and 1065; and/or a CDR2 having at 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a sequence selected from SEQ ID NOs. 686, 696, 706, 716, 726, 736, 746, 756, 766, 776, 786, 796, 806, 816, 826, 836, 846, 856, 866, 876, 886, 896, 906, 916, 926, 936, 946, 956, 966, 976, 986, 996, 1006, 1016, 1026, 1036, 1046, 1056, and 1066, (eg a CDR2 sequence disclosed in Table 13.3); and/or in particular eg an amino acid sequence of a light chain CDR2 as shown in Table 13.3 for the corresponding light chain CDR2 of an antibody selected from any one of the antibodies of the group consisting of D-101 to D-116, and D-201 to D-223, preferably D-114, D-115, or D-116 (eg, D-114), and/or selected from D-222 or D-223, preferably D-222, such as a sequence selected from SEQ ID Nos.: 816, 826, 836, 1056 and 1066.

In other embodiments of the present invention, an ABP of the invention can comprise an antibody variable chain sequence having at least 80%, 85%, 90%; or 95% (preferably at least 90%) sequence identity to, or having no more than fifteen, fourteen, thirteen, twelve or eleven (eg, for variable light chain), such as having no more than about 20, 18, 16, 14, or 12, or no more than ten, nine, eight, seven, six, five, four, three, two or one, preferably no more than three, two or one amino acid substitution(s), deletion(s) or insertion(s) (in particular, substitution(s)) compared to, a sequence selected from SEQ ID NOs. 394, 398, 404, 408, 414, 418, 424, 428, 434, 438, 444, 448, 454, 458, 464, 468, 474, 478, 484, 488, 494, 498, 504, 508, 514, 518, 524, 528, 534, 538, 544, 548, 554, 558, 564, 568, 574, 578, 584, 588, 594, 598, 604, 608, 614, 618, 624, 628, 634, 638, 644, 648, 654, 658, 664, 668, 674, and 678 (eg, a VH or VL sequence disclosed in Table 13.1A); and/or in particular eg compared to an amino acid sequence of an antibody variable chain sequence as shown in Table 13.1A for the corresponding heavy or light variable chain of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007.

In other embodiments of the present invention, a further ABP of the invention can comprise an antibody variable chain sequence having at least 80%, 85%, 90%; or 95% (preferably at least 90%) sequence identity to, or having no more than fifteen, fourteen, thirteen, twelve or eleven (eg, for variable light chain), such as having no more than about 20, 18, 16, 14, or 12, or no more than ten, nine, eight, seven, six, five, four, three, two or one, preferably no more than three, two or one amino acid substitution(s), deletion(s) or insertion(s) (in particular, substitution(s)) compared to, a sequence selected from SEQ ID NOs. 684, 688, 694, 698, 704, 708, 714, 718, 724, 728, 734, 738, 744, 748, 754, 758, 764, 768, 774, 778, 784, 788, 794, 798, 804, 808, 814, 818, 824, 828, 834, 838, 844, 848, 854, 858, 864, 868, 874, 878, 884, 888, 894, 898, 904, 908, 914, 918, 924, 928, 934, 938, 944, 948, 954, 958, 964, 968, 974, 978, 984, 988, 994, 998, 1004, 1008, 1014, 1018, 1024, 1028, 1034, 1038, 1044, 1048, 1054, 1058, 1064, and 1068 (eg, a VH or VL sequence disclosed in Table 13.3); and/or in particular eg compared to an amino acid sequence of an antibody variable chain sequence as shown in Table 13.3 for the corresponding heavy or light variable chain of an antibody selected from any one of the antibodies of the group consisting of: D-101 to D-116, and D-201 to D-223, preferably D-114, D-115, or D-116 (eg, D-114), and/or selected from D-222 or D-223, preferably D-222.

In particular embodiments of the invention, an ABP of the invention comprises an antigen binding fragment of an antibody, wherein the antigen binding fragment comprises CDR1, CDR2 and CDR3. In certain of such embodiments, the CDR1 is selected from those disclosed in Table 13.1A, the CDR2 is selected from those disclosed in Table 13.1A and the CDR3 is selected from those disclosed in Table 13.1A (eg, the CDR1, CDR2 and CDR3 are selected from the CDR1, CDR2 and CDR3 sequences having the respective amino acid sequences of SEQ ID Nos. 391, 392, 393, or 395, 396, 397, or 401, 402, 403, or 405, 406, 407, or 411, 412, 413, or 415, 416, 417, or 421, 422, 423, or 425, 426, 427, or 431, 432, 433, or 435, 436, 437, or 441, 442, 443, or 445, 446, 447, or 451, 452, 453, or 455, 456, 457, or 461, 462, 463, or 465, 466, 467, or 471, 472, 473, or 475, 476, 477, or 481, 482, 483, or 485, 486, 487, or 491, 492, 493, or 495, 496, 497, or 501, 502, 503, or 505, 506, 507, or 511, 512, 513, or 515, 516, 517, or 521, 522, 523, or 525, 526, 527, or 531, 532, 533, or 535, 536, 537, or 541, 542, 543, or 545, 546, 547, or 551, 552, 553, or 555, 556, 557, or 561, 562, 563, or 565, 566, 567, or 571, 572, 573, or 575, 576, 577, or 581, 582, 583, or 585, 586, 587, or 591, 592, 593, or 595, 596, 597, or 601, 602, 603, or 605, 606, 607, or 611, 612, 613, or 615, 616, 617, or 621, 622, 623, or 625, 626, 627, or 631, 632, 633, or 635, 636, 637, or 641, 642, 643, or 645, 646, 647, or 651, 652, 653, or 655, 656, 657, or 661, 662, 663, or 665, 666, 667, or 671, 672, 673, or 675, 676, 677; and/or in particular eg are amino acid sequences of a CDR1, CDR2 and CDR3 sequence and/or a CDR1, CDR2 and CDR3 sequence as shown in Table 13.1A for the corresponding CDR1, CDR2 and CDR3 of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007; in each case independently, optionally with no more than eight, seven, six, five or four (eg, for L-CDR3), or with no more than three or two, preferably no more than one, amino acid substitution(s), insertion(s) or deletion(s) (in particular, substitution(s)) compared to these sequences.

In particular embodiments of the invention, a further ABP of the invention comprises an antigen binding fragment of an antibody, wherein the antigen binding fragment comprises CDR1, CDR2 and CDR3. In certain of such embodiments, the CDR1 is selected from those disclosed in Table 13.3, the CDR2 is selected from those disclosed in Table 13.3 and the CDR3 is selected from those disclosed in Table 13.3 (eg, the CDR1, CDR2 and CDR3 are selected from the CDR1, CDR2 and CDR3 sequences having the respective amino acid sequences of SEQ ID Nos. 681, 682, 683, or 685, 686, 687, or 691, 692, 693, or 695, 696, 697, or 701, 702, 703, or 705, 706, 707, or 711, 712, 713, or 715, 716, 717, or 721, 722, 723, or 725, 726, 727, or 731, 732, 733, or 735, 736, 737, or 741, 742, 743, or 745, 746, 747, or 751, 752, 753, or 755, 756, 757, or 761, 762, 763, or 765, 766, 767, or 771, 772, 773, or 775, 776, 777, or 781, 782, 783, or 785, 786, 787, or 791, 792, 793, or 795, 796, 797, or 801, 802, 803, or 805, 806, 807, or 811, 812, 813, or 815, 816, 817, or 821, 822, 823, or 825, 826, 827, or 831, 832, 833, or 835, 836, 837, or 841, 842, 843, or 845, 846, 847, or 851, 852, 853, or 855, 856, 857, or 861, 862, 863, or 865, 866, 867, or 871, 872, 873, or 875, 876, 877, or 881, 882, 883, or 885, 886, 887, or 891, 892, 893, or 895, 896, 897, or 901, 902, 903, or 905, 906, 907, or 911, 912, 913, or 915, 916, 917, or 921, 922, 923, or 925, 926, 927, or 931, 932, 933, or 935, 936, 937, or 941, 942, 943, or 945, 946, 947, or 951, 952, 953, or 955, 956, 957, or 961, 962, 963, or 965, 966, 967, or 971, 972, 973, or 975, 976, 977, or 981, 982, 983, or 985, 986, 987, or 991, 992, 993, or 995, 996, 997, or 1001, 1002, 1003, or 1005, 1006, 1007, or 1011, 1012, 1013, or 1015, 1016, 1017, or 1021, 1022, 1023, or 1025, 1026, 1027, or 1031, 1032, 1033, or 1035, 1036, 1037, or 1041, 1042, 1043, or 1045, 1046, 1047, or 1051, 1052, 1053, or 1055, 1056, 1057, or 1061, 1062, 1063, or 1065, 1066, 1067; and/or in particular eg are amino acid sequences of a CDR1, CDR2 and CDR3 sequence and/or a CDR1, CDR2 and CDR3 sequence as shown in Table 13.3 for the corresponding CDR1, CDR2 and CDR3 of an antibody selected from any one of the antibodies of the group consisting of: D-101 to D-116, and D-201 to D-223, preferably D-114, D-115, or D-116 (eg, D-114), and/or selected from D-222 or D-223, preferably D-222; in each case independently, optionally with no more than eight, seven, six, five or four (eg, for L-CDR3), or with no more than three or two, preferably no more than one, amino acid substitution(s), insertion(s) or deletion(s) (in particular, substitution(s)) compared to these sequences.

In further particular embodiments of the present invention, an ABP of the invention can comprise an antibody heavy chain variable region CDR1, CDR2, and CDR3, and/or an antibody light chain variable region CDR1, CDR2, and CDR3, wherein the CDR1 has an amino acid sequence of a heavy or light chain CDR1 shown in Table 13.1A (eg has an amino acid sequence selected from the list consisting of SEQ ID No 391, 395, 401, 405, 411, 415, 421, 425, 431, 435, 441, 445, 451, 455, 461, 465, 471, 475, 481, 485, 491, 495, 501, 505, 511, 515, 521, 525, 531, 535, 541, 545, 551, 555, 561, 565, 571, 575, 581, 585, 591, 595, 601, 605, 611, 615, 621, 625, 631, 635, 641, 645, 651, 655, 661, 665, 671 and 675; and/or in particular eg has an amino acid sequence of an antibody heavy or light chain variable region CDR1 sequence as shown in Table 13.1A for the corresponding heavy or light chain CDR1 of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007), and wherein the CDR2 has an amino acid sequence of a heavy or light chain CDR2 shown in Table 13.1A (eg has an amino acid sequence selected from the list consisting of SEQ ID No 392, 396, 402, 406, 412, 416, 422, 426, 432, 436, 442, 446, 452, 456, 462, 466, 472, 476, 482, 486, 492, 496, 502, 506, 512, 516, 522, 526, 532, 536, 542, 546, 552, 556, 562, 566, 572, 576, 582, 586, 592, 596, 602, 606, 612, 616, 622, 626, 632, 636, 642, 646, 652, 656, 662, 666, 672 and 676; and/or in particular eg has an amino acid sequence of an antibody heavy or light chain variable region CDR2 sequence as shown in Table 13.1A for the corresponding heavy or light chain CDR2 of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007), and wherein the CDR3 has an amino acid sequence of a heavy or light chain CDR3 shown in Table 13.1A (eg has an amino acid sequence selected from the list consisting of SEQ ID No: 393, 397, 403, 407, 413, 417, 423, 427, 433, 437, 443, 447, 453, 457, 463, 467, 473, 477, 483, 487, 493, 497, 503, 507, 513, 517, 523, 527, 533, 537, 543, 547, 553, 557, 563, 567, 573, 577, 583, 587, 593, 597, 603, 607, 613, 617, 623, 627, 633, 637, 643, 647, 653, 657, 663, 667, 673, and 677; and/or in particular eg has an amino acid sequence of an antibody heavy or light chain variable region CDR3 sequence as shown in Table 13.1A for the corresponding heavy or light chain CDR3 of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007); in each case independently, optionally with no more than eight, seven, six, five or four (eg, for L-CDR3), or with no more than three or two, preferably no more than one, amino acid substitution(s), insertion(s) or deletion(s) (in particular, substitution(s)) compared to these sequences.

In further particular embodiments of the present invention, a further ABP of the invention can comprise an antibody heavy chain variable region CDR1, CDR2, and CDR3, and/or an antibody light chain variable region CDR1, CDR2, and CDR3, wherein the CDR1 has an amino acid sequence of a heavy or light chain CDR1 shown in Table 13.3 (eg has an amino acid sequence selected from the list consisting of SEQ ID No 681, 685, 691, 695, 701, 705, 711, 715, 721, 725, 731, 735, 741, 745, 751, 755, 761, 765, 771, 775, 781, 785, 791, 795, 801, 805, 811, 815, 821, 825, 831, 835, 841, 845, 851, 855, 861, 865, 871, 875, 881, 885, 891, 895, 901, 905, 911, 915, 921, 925, 931, 935, 941, 945, 951, 955, 961, 965, 971, 975, 981, 985, 991, 995, 1001, 1005, 1011, 1015, 1021, 1025, 1031, 1035, 1041, 1045, 1051, 1055, 1061, and 1065; and/or in particular eg has an amino acid sequence of an antibody heavy or light chain variable region CDR1 sequence as shown in Table 13.3 for the corresponding heavy or light chain CDR1 of an antibody selected from any one of the antibodies of the group consisting of: D-101 to D-116, and D-201 to D-223, preferably D-114, D-115, or D-116 (eg, D-114), and/or selected from D-222 or D-223, preferably D-222, and wherein the CDR2 has an amino acid sequence of a heavy or light chain CDR2 shown in Table 13.3 (eg has an amino acid sequence selected from the list consisting of SEQ ID No 682, 686, 692, 696, 702, 706, 712, 716, 722, 726, 732, 736, 742, 746, 752, 756, 762, 766, 772, 776, 782, 786, 792, 796, 802, 806, 812, 816, 822, 826, 832, 836, 842, 846, 852, 856, 862, 866, 872, 876, 882, 886, 892, 896, 902, 906, 912, 916, 922, 926, 932, 936, 942, 946, 952, 956, 962, 966, 972, 976, 982, 986, 992, 996, 1002, 1006, 1012, 1016, 1022, 1026, 1032, 1036, 1042, 1046, 1052, 1056, 1062, and 1066; and/or in particular eg has an amino acid sequence of an antibody heavy or light chain variable region CDR2 sequence as shown in Table 13.3 for the corresponding heavy or light chain CDR2 of an antibody selected from any one of the antibodies of the group consisting of: D-101 to D-116, and D-201 to D-223, preferably D-114, D-115, or D-116 (eg, D-114), and/or selected from D-222 or D-223, preferably D-222, and wherein the CDR3 has an amino acid sequence of a heavy or light chain CDR3 shown in Table 13.3 (eg has an amino acid sequence selected from the list consisting of SEQ ID No: 683, 687, 693, 697, 703, 707, 713, 717, 723, 727, 733, 737, 743, 747, 753, 757, 763, 767, 773, 777, 783, 787, 793, 797, 803, 807, 813, 817, 823, 827, 833, 837, 843, 847, 853, 857, 863, 867, 873, 877, 883, 887, 893, 897, 903, 907, 913, 917, 923, 927, 933, 937, 943, 947, 953, 957, 963, 967, 973, 977, 983, 987, 993, 997, 1003, 1007, 1013, 1017, 1023, 1027, 1033, 1037, 1043, 1047, 1053, 1057, 1063, and 1067; and/or in particular eg has an amino acid sequence of an antibody heavy or light chain variable region CDR3 sequence as shown in Table 13.1A for the corresponding heavy or light chain CDR3 of an antibody selected from any one of the antibodies of the group consisting of: D-101 to D-116, and D-201 to D-223, preferably D-114, D-115, or D-116 (eg, D-114), and/or selected from D-222 or D-223, preferably D-222; in each case independently, optionally with no more than eight, seven, six, five or four (eg, for L-CDR3), or with no more than three or two, preferably no more than one, amino acid substitution(s), insertion(s) or deletion(s) (in particular, substitution(s)) compared to these sequences.

In preferred of such embodiments, the ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise CDR1 to CDR3 sequences in a combination selected from any of the combinations of heavy chain CDRs shown in Table B.2 and/or selected from any of the combinations of light chain CDRs shown in Table B.2 (in each case, combinations CDRs-C001 to CDRs-C-029; and in particular, such heavy chain CDRs and/or light chain CDRs combinations of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007); in each case independently, optionally with no more than eight, seven, six, five or four (eg, for L-CDR3), or with no more than three or two, preferably no more than one, amino acid substitution(s), insertion(s) or deletion(s) (in particular, substitution(s)) compared to these sequences. Preferably, the combination of both the heavy chain CDRs and the light chain CDRs is one selected from a row marked by any one of the combinations CDRs-C-001 to CDRs-AC029 (in particular, such heavy chain CDRs and the light chain CDRs combinations of an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007), in each CDR independently optionally with no more than eight, seven, six, five or four (eg, for L-CDR3), or with no more than three or two, preferably no more than one, amino acid substitution(s), insertion(s) or deletion(s) (in particular, substitution(s)) compared to these sequences.

TABLE B.2
preferred combinations of heavy chain CDRs and preferred
combinations of light chain CDRs

| | Heavy Chain | | | Light Chain | | |
|---|---|---|---|---|---|---|
| Combination | CDR1 to CDR3 | | | CDR1 to CDR3 | | |
| (ID) | (SEQ ID NO) | | | (SEQ ID NO) | | |
| CDRs-C-001 | 391 | 392 | 393 | 395 | 396 | 397 |
| CDRs-C-002 | 401 | 402 | 403 | 405 | 406 | 407 |
| CDRs-C-003 | 411 | 412 | 413 | 415 | 416 | 417 |
| CDRs-C-004 | 421 | 422 | 423 | 425 | 426 | 427 |
| CDRs-C-005 | 431 | 432 | 433 | 435 | 436 | 437 |
| CDRs-C-006 | 441 | 442 | 443 | 445 | 446 | 447 |
| CDRs-C-007 | 451 | 452 | 453 | 455 | 456 | 457 |
| CDRs-C-008 | 461 | 462 | 463 | 465 | 466 | 467 |
| CDRs-C-009 | 471 | 472 | 473 | 475 | 476 | 477 |
| CDRs-C-010 | 481 | 482 | 483 | 485 | 486 | 487 |
| CDRs-C-011 | 491 | 492 | 493 | 495 | 496 | 497 |
| CDRs-C-012 | 501 | 502 | 503 | 505 | 506 | 507 |
| CDRs-C-013 | 511 | 512 | 513 | 515 | 516 | 517 |
| CDRs-C-014 | 521 | 522 | 523 | 525 | 526 | 527 |
| CDRs-C-015 | 531 | 532 | 533 | 535 | 536 | 537 |
| CDRs-C-016 | 541 | 542 | 543 | 545 | 546 | 547 |
| CDRs-C-017 | 551 | 552 | 553 | 555 | 556 | 557 |
| CDRs-C-018 | 561 | 562 | 563 | 565 | 566 | 567 |
| CDRs-C-019 | 571 | 572 | 573 | 575 | 576 | 577 |
| CDRs-C-020 | 581 | 582 | 583 | 585 | 586 | 587 |
| CDRs-C-021 | 591 | 592 | 593 | 595 | 596 | 597 |
| CDRs-C-022 | 601 | 602 | 603 | 605 | 606 | 607 |
| CDRs-C-023 | 611 | 612 | 613 | 615 | 616 | 617 |
| CDRs-C-024 | 621 | 622 | 623 | 625 | 626 | 627 |
| CDRs-C-025 | 631 | 632 | 633 | 635 | 636 | 637 |
| CDRs-C-026 | 641 | 642 | 643 | 645 | 646 | 647 |
| CDRs-C-027 | 651 | 652 | 653 | 655 | 656 | 657 |
| CDRs-C-028 | 661 | 662 | 663 | 665 | 666 | 667 |
| CDRs-C-029 | 671 | 672 | 673 | 675 | 676 | 677 |

In further preferred of such embodiments, the further ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise CDR1 to CDR3 sequences in a combination selected from any of the combinations of heavy chain CDRs shown in Table 13.3 and/or selected from any of the combinations of light chain CDRs shown in Table B.3; and in particular, such heavy chain CDRs and/or light chain CDRs combinations of an antibody selected from any one of the antibodies of the group consisting of: CDRs-D-101 to CDRs-D-116, and CDRs-D-201 to CDRs-D-223, preferably CDRs-D-114, CDRs-D-115, or CDRs-D-116 (eg, CDRs-D-114), and/or selected from CDRs-D-222 or CDRs-D-223, preferably CDRs-D-222; in each case independently, optionally with no more than eight, seven, six, five or four (eg, for L-CDR3), or with no more than three or two, preferably no more than one, amino acid substitution(s), insertion(s) or deletion(s) (in particular, substitution(s)) compared to these sequences. Preferably, the combination of both the heavy chain CDRs and the light chain CDRs is one selected from a row marked by any one of the combinations CDRs-D-101 to CDRs-D-116, and CDRs-D-201 to CDRs-D-223 (in particular, such heavy chain CDRs and the light chain CDRs combinations of an antibody selected from any one of the antibodies of the group consisting of: CDRs-D-101 to CDRs-D-116, and CDRs-D-201 to CDRs-D-223, preferably CDRs-D-114, CDRs-D-115, or CDRs-D-116 (eg, CDRs-D-114), and/or selected from CDRs-D-222 or CDRs-D-223, preferably CDRs-D-222, in each CDR independently optionally with no more than eight, seven, six, five or four (eg, for L-CDR3), or with no more than three or two, preferably no more than one, amino acid substitution(s), insertion(s) or deletion(s) (in particular, substitution(s)) compared to these sequences.

TABLE B.3
preferred combinations of heavy chain CDRs and preferred
combinations of light chain CDRs

| | Heavy Chain | | | Light Chain | | |
|---|---|---|---|---|---|---|
| Combination | CDR1 to CDR3 | | | CDR1 to CDR3 | | |
| (ID) | (SEQ ID NO) | | | (SEQ ID NO) | | |
| CDRs-D-101 | 681 | 682 | 683 | 685 | 686 | 687 |
| CDRs-D-102 | 691 | 692 | 693 | 695 | 696 | 697 |
| CDRs-D-103 | 701 | 702 | 703 | 705 | 706 | 707 |
| CDRs-D-104 | 711 | 712 | 713 | 715 | 716 | 717 |
| CDRs-D-105 | 721 | 722 | 723 | 725 | 726 | 727 |
| CDRs-D-106 | 731 | 732 | 733 | 735 | 736 | 737 |
| CDRs-D-107 | 741 | 742 | 743 | 745 | 746 | 747 |
| CDRs-D-108 | 751 | 752 | 753 | 755 | 756 | 757 |
| CDRs-D-109 | 761 | 762 | 763 | 765 | 766 | 767 |
| CDRs-D-110 | 771 | 772 | 773 | 775 | 776 | 777 |
| CDRs-D-111 | 781 | 782 | 783 | 785 | 786 | 787 |
| CDRs-D-112 | 791 | 792 | 793 | 795 | 796 | 797 |
| CDRs-D-113 | 801 | 802 | 803 | 805 | 806 | 807 |
| CDRs-D-114 | 811 | 812 | 813 | 815 | 816 | 817 |
| CDRs-D-115 | 821 | 822 | 823 | 825 | 826 | 827 |
| CDRs-D-116 | 831 | 832 | 833 | 835 | 836 | 837 |
| CDRs-D-201 | 841 | 842 | 843 | 845 | 846 | 847 |
| CDRs-D-202 | 851 | 852 | 853 | 855 | 856 | 857 |
| CDRs-D-203 | 861 | 862 | 863 | 865 | 866 | 867 |
| CDRs-D-204 | 871 | 872 | 873 | 875 | 876 | 877 |
| CDRs-D-205 | 881 | 882 | 883 | 885 | 886 | 887 |
| CDRs-D-206 | 891 | 892 | 893 | 895 | 896 | 897 |
| CDRs-D-207 | 901 | 902 | 903 | 905 | 906 | 907 |
| CDRs-D-208 | 911 | 912 | 913 | 915 | 916 | 917 |
| CDRs-D-209 | 921 | 922 | 923 | 925 | 926 | 927 |
| CDRs-D-210 | 931 | 932 | 933 | 935 | 936 | 937 |
| CDRs-D-211 | 941 | 942 | 943 | 945 | 946 | 947 |
| CDRs-D-212 | 951 | 952 | 953 | 955 | 956 | 957 |
| CDRs-D-213 | 961 | 962 | 963 | 965 | 966 | 967 |
| CDRs-D-214 | 971 | 972 | 973 | 975 | 976 | 977 |
| CDRs-D-215 | 981 | 982 | 983 | 985 | 986 | 987 |
| CDRs-D-216 | 991 | 992 | 993 | 995 | 996 | 997 |
| CDRs-D-217 | 1001 | 1002 | 1003 | 1005 | 1006 | 1007 |
| CDRs-D-218 | 1011 | 1012 | 1013 | 1015 | 1016 | 1017 |
| CDRs-D-219 | 1021 | 1022 | 1023 | 1025 | 1026 | 1027 |
| CDRs-D-220 | 1031 | 1032 | 1033 | 1035 | 1036 | 1037 |
| CDRs-D-221 | 1041 | 1042 | 1043 | 1045 | 1046 | 1047 |
| CDRs-D-222 | 1051 | 1052 | 1053 | 1055 | 1056 | 1057 |
| CDRs-D-223 | 1061 | 1062 | 1063 | 1065 | 1066 | 1067 |

In other preferred embodiments of the invention, the ABP may be an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown in Table C.2 (eg, selected from any of the variable chain combinations Chains-C-001 to Chains-C-029; and in particular, the variable chain combinations of an antibody selected from any one of the antibodies of the group consisting of: C-002, C-003, C-004, C-005, C-006, C-010, C-Oil, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007) in each case independently, optionally with no more than fifteen, fourteen, thirteen, twelve or eleven (eg, for variable light chain), such with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences.

TABLE C.2
preferred combinations of heavy and light chain variably domains

| | Heavy Chain | Light Chain |
|---|---|---|
| Combination | Variable Domain | Variable Domain |
| (ID) | (SEQ ID NO) | (SEQ ID NO) |
| Chains-C-001 | 394 | 398 |
| Chains-C-002 | 404 | 408 |
| Chains-C-003 | 414 | 418 |
| Chains-C-004 | 424 | 428 |
| Chains-C-005 | 434 | 438 |
| Chains-C-006 | 444 | 448 |
| Chains-C-007 | 454 | 458 |
| Chains-C-008 | 464 | 468 |
| Chains-C-009 | 474 | 478 |
| Chains-C-010 | 484 | 488 |
| Chains-C-011 | 494 | 498 |
| Chains-C-012 | 504 | 508 |
| Chains-C-013 | 514 | 518 |
| Chains-C-014 | 524 | 528 |
| Chains-C-015 | 534 | 538 |
| Chains-C-016 | 544 | 548 |
| Chains-C-017 | 554 | 558 |
| Chains-C-018 | 564 | 568 |
| Chains-C-019 | 574 | 578 |
| Chains-C-020 | 584 | 588 |
| Chains-C-021 | 594 | 598 |
| Chains-C-022 | 604 | 608 |
| Chains-C-023 | 614 | 618 |
| Chains-C-024 | 624 | 628 |
| Chains-C-025 | 634 | 638 |
| Chains-C-026 | 644 | 648 |
| Chains-C-027 | 654 | 658 |
| Chains-C-028 | 664 | 668 |
| Chains-C-029 | 674 | 678 |

In other further preferred embodiments of the invention, the further ABP may be an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown in Table C.3 (eg, selected from any of the variable chain combinations Chains-D-101 to Chains-D-116, and Chains-D-201 to Chains-D-223; and in particular, the variable chain combinations of an antibody selected from any one of the antibodies of the group consisting of: Chains-D-101 to Chains-D-116, and Chains-D-201 to Chains-D-223, preferably Chains-D-114, Chains-D-115, or Chains-D-116 (eg, Chains-D-114), and/or selected from Chains-D-222 or Chains-D-223, preferably Chains-D-222, in each case independently, optionally with no more than fifteen, fourteen, thirteen, twelve or eleven (eg, for variable light chain), such with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences.

TABLE C.3
preferred combinations of heavy and light chain variably domains

| | Heavy Chain | Light Chain |
|---|---|---|
| Combination | Variable Domain | Variable Domain |
| (ID) | (SEQ ID NO) | (SEQ ID NO) |
| Chains-D-101 | 684 | 688 |
| Chains-D-102 | 694 | 698 |
| Chains-D-103 | 704 | 708 |
| Chains-D-104 | 714 | 718 |
| Chains-D-105 | 724 | 728 |
| Chains-D-106 | 734 | 738 |
| Chains-D-107 | 744 | 748 |
| Chains-D-108 | 754 | 758 |
| Chains-D-109 | 764 | 768 |
| Chains-D-110 | 774 | 778 |
| Chains-D-111 | 784 | 788 |
| Chains-D-112 | 794 | 798 |
| Chains-D-113 | 804 | 808 |
| Chains-D-114 | 814 | 818 |
| Chains-D-115 | 824 | 828 |
| Chains-D-116 | 834 | 838 |
| Chains-D-201 | 844 | 848 |
| Chains-D-202 | 854 | 858 |
| Chains-D-203 | 864 | 868 |
| Chains-D-204 | 874 | 878 |
| Chains-D-205 | 884 | 888 |
| Chains-D-206 | 894 | 898 |
| Chains-D-207 | 904 | 908 |
| Chains-D-208 | 914 | 918 |
| Chains-D-209 | 924 | 928 |
| Chains-D-210 | 934 | 938 |
| Chains-D-211 | 944 | 948 |
| Chains-D-212 | 954 | 958 |
| Chains-D-213 | 964 | 968 |
| Chains-D-214 | 974 | 978 |
| Chains-D-215 | 984 | 988 |
| Chains-D-216 | 994 | 998 |
| Chains-D-217 | 1004 | 1008 |
| Chains-D-218 | 1014 | 1018 |
| Chains-D-219 | 1024 | 1028 |
| Chains-D-220 | 1034 | 1038 |
| Chains-D-221 | 1044 | 1048 |
| Chains-D-222 | 1054 | 1058 |
| Chains-D-223 | 1064 | 1068 |

In preferred of such embodiments, the ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise CDR1 to CDR3 sequences selected from the sequences shown in SEQ ID NO: 414, 424 and 434 (eg, 434), or selected from the sequences shown in SEQ ID NO: 394 or 454; and at least one, preferably both, of the antibody light chain sequences comprise CDR1 to CDR3 sequences in a combination selected from any of the combinations of light chain CDRs shown in Table B.2; in each case independently, optionally with no more than three or two, preferably no more than one, amino acid substitution(s), insertion(s) or deletion(s) (in particular, substitution(s)) compared to these sequences. Most preferably is a combination indicated for rows CDRs-C-003, CDRs-C-004 or CDRs-C-005 (eg, CDR-C-005), or is a combination indicated for rows CDRs-C-001 or CDRs-C-007.

In preferred of such embodiments, the ABP may be an antibody, or an antigen binding fragment thereof, composed of at least one, preferably two, antibody heavy chain sequences, and at least one, preferably two, antibody light chain sequences, wherein at least one, preferably both, of the antibody light chain sequences comprise CDR1 to CDR3 sequences selected from the sequences shown in SEQ ID NO: 415, 416 and 417; or 425, 426 and 427; or 435, 436 and 437 (eg, 435, 436 and 437), or selected from the sequences shown in SEQ ID NO: 395, 396 and 397; or 455, 456 and 457; and at least one, preferably both, of the antibody heavy chain sequences comprise CDR1 to CDR3 sequences in a combination selected from any of the combinations of heavy chain CDRs shown in Table B.2; in each case independently, optionally with no more than eight, seven, six, five or four (eg for L-CDR3), such as no more than three or two, preferably no more than one, amino acid substitution(s), insertion(s) or deletion(s) (in particular, substitution(s)) compared to these sequences.

In preferred of such embodiments, the ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise CDR1 to CDR3 sequences in the combination of the combinations of heavy and light chain CDRs shown in Table B.2 rows: CDRs-C-003, CDRs-C-004 or CDRs-C-005 (eg, CDR-C-005), or is a combination indicated for rows CDRs-C-001 or CDRs-C-007; in each case independently, optionally with no more than three or two, preferably no more than one, amino acid substitution(s), insertion(s) or deletion(s) (in particular, substitution(s)) compared to these sequences.

In other preferred embodiments of the invention, the ABP may be an antibody, or an antigen binding fragment thereof, composed of at least one, preferably two, antibody heavy chain sequence, and at least one, preferably two, antibody light chain sequence, wherein the at least one, preferably two, antibody heavy chain sequence comprises a variable region sequence selected from the sequences according to SEQ ID NO: 411, 412 and 413; or 421, 422 and 432; or 431, 432 and 433 (eg, 431, 432 and 433), or selected from the sequences shown in SEQ ID NO: 391, 392 and 393; or 451, 452 and 453; and wherein the least one, preferably two, antibody light chain sequence comprises a light chain variable domain shown in Table C.2; in each case independently, optionally with no more than fifteen, fourteen, thirteen, twelve or eleven (eg, for variable light chain), or with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences.

In other preferred embodiments of the invention, the ABP may be an antibody, or an antigen binding fragment thereof, composed of at least one, preferably two, antibody heavy chain sequence, and at least one, preferably two, antibody light chain sequence, wherein the at least one, preferably two, antibody light chain sequence comprises a variable region sequence selected from the sequences according to SEQ ID NO: 419, 429 and 439 (eg, 439), or selected from the sequences shown in SEQ ID NO: 399 and 459; and wherein the least one, preferably two, antibody heavy chain sequence comprises a heavy chain variable domain shown in Table C.2; in each case independently, optionally with no more than fifteen, fourteen, thirteen, twelve or eleven (eg, for variable light chain), or with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences.

In other preferred embodiments of the invention, the ABP may be an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown in Table C.2 rows Chains-C-003, Chains-C-004 or Chains-C-005 (eg, Chains-C-005, or is a combination indicated for rows Chains-C-001 or Chains-C-007; in each case independently, optionally with no more than fifteen, fourteen, thirteen, twelve or eleven (eg, for variable light chain), or with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences.

In preferred of such embodiments, the further ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise CDR1 to CDR3 sequences shown in SEQ ID NO: 811, 812, and 813; and at least one, preferably both, of the antibody light chain sequences comprise CDR1 to CDR3 sequences shown in SEQ ID NO: 815, 816, and 817; in each case independently, optionally with no more than three or two, preferably no more than one, amino acid substitution(s), insertion(s) or deletion(s) (in particular, substitution(s)) compared to these sequences.

In preferred of such embodiments, the further ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise a variable domain sequence shown in SEQ ID NO: 814; and at least one, preferably both, of the antibody light chain sequences comprise a variable domain sequences shown in SEQ ID NO: 818; in each case independently, optionally with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences.

In preferred of such embodiments, the further ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise CDR1 to CDR3 sequences shown in SEQ ID NO: 821, 822, and 823; and at least one, preferably both, of the antibody light chain sequences comprise CDR1 to CDR3 sequences shown in SEQ ID NO: 825, 826, and 827; in each case independently, optionally with no more than three or two, preferably no more than one, amino acid substitution(s), insertion(s) or deletion(s) (in particular, substitution(s)) compared to these sequences.

In preferred of such embodiments, the further ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise a variable domain sequence shown in SEQ ID NO: 824; and at least one, preferably both, of the antibody light chain sequences comprise a variable domain sequences shown in SEQ ID NO: 828; in each case independently, optionally with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences.

In preferred of such embodiments, the further ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise CDR1 to CDR3 sequences shown in SEQ ID NO: 831, 832, and 833; and at least one, preferably both, of the antibody light chain sequences comprise CDR1 to CDR3 sequences shown in SEQ ID NO: 835, 836, and 837; in each case independently, optionally with no more than three or two, preferably no more than one, amino acid substitution(s), insertion(s) or deletion(s) (in particular, substitution(s)) compared to these sequences.

In preferred of such embodiments, the further ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise a variable domain sequence shown in SEQ ID NO: 834; and at least one, preferably both, of the antibody light chain sequences comprise a variable domain sequences shown in SEQ ID NO: 838; in each case independently, optionally with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences.

In preferred of such embodiments, the further ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise CDR1 to CDR3 sequences shown in SEQ ID NO: 1051, 1052, and 1053; and at least one, preferably both, of the antibody light chain sequences comprise CDR1 to CDR3 sequences shown in SEQ ID NO: 1055, 1056, and 1057; in each case independently, optionally with no more than three or two, preferably no more than one, amino acid substitution(s), insertion(s) or deletion(s) (in particular, substitution(s)) compared to these sequences.

In preferred of such embodiments, the further ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise a variable domain sequence shown in SEQ ID NO: 1054; and at least one, preferably both, of the antibody light chain sequences comprise a variable domain sequences shown in SEQ ID NO: 1058; in each case independently, optionally with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences.

In preferred of such embodiments, the further ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise CDR1 to CDR3 sequences shown in SEQ ID NO: 1061, 1062, and 1063; and at least one, preferably both, of the antibody light chain sequences comprise CDR1 to CDR3 sequences shown in SEQ ID NO: 1065, 1066, and 1067; in each case independently, optionally with no more than three or two, preferably no more than one, amino acid substitution(s), insertion(s) or deletion(s) (in particular, substitution(s)) compared to these sequences.

In preferred of such embodiments, the further ABP may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise a variable domain sequence shown in SEQ ID NO: 1064; and at least one, preferably both, of the antibody light chain sequences comprise a variable domain sequences shown in SEQ ID NO: 1068; in each case independently, optionally with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences.

In particularly preferred embodiment, an ABP of the invention can comprise a combination of heavy chain CDR1, CDR2 and CDR3 sequences and a combination of light chain CDR1, CDR2 and CDR3 sequences in the combination shown by antibody C-003, C-004 or C-005 (eg, C-005), such as shown in Table B.2 by row CDRs-C-005 (eg, heavy chain CDR1, CDR2 and CDR3 having a sequence shown by SEQ ID Nos, 431, 432 and 433, respectively, and light chain CDR1, CDR2 and CDR3 having a sequence shown by SEQ ID Nos, 435, 436 and 437, respectively), in each CDR independently, optionally with no more than eight, seven, six, five or four (eg for L-CDR3), such as with no more than three or two, preferably no more than one, amino acid substitution(s), insertion(s) or deletion(s) (in particular, substitution(s)) compared to these sequences. In another particularly preferred embodiment, an ABP of the invention can be an antibody, or an antigen binding fragment thereof, composed of at least one, preferably two, antibody heavy chain sequences, and at least one, preferably two, antibody light chain sequences, wherein at least one, preferably both, of the antibody heavy chain sequences each comprises heavy chain CDR1 to CDR3 sequences in the combination CDRs-C-005 and at least one, preferably both, of the antibody light chain sequences each comprises light chain CDR1 to CDR3 sequences in the combination shown in the row of Table B.2 marked by CDRs-C-005, in each CDR independently, optionally with no more than one amino acid substitution(s), insertion(s) or deletion(s) (in particular, substitution(s)) compared to these sequences. In yet another particularly preferred embodiment, an ABP of the invention can be an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown the row of Table B.2 marked by CDRs-C-005. In each of such particularly preferred embodiments of the ABP, optionally, the ABP is able to inhibit the binding of an interacting protein (eg VSIR protein or a variant thereof) to an IgC2 domain of (or an IgV domain of) IGSF11 protein or a variant thereof with an IC50 of 20 nM or less or 10 nM or less, such as 5 nM or less, or preferably 2 nM or less, or an IC50 about equimolar to the concentration of the binding partner (eg. VSIR protein). Such IC50s can be determined using the methods described elsewhere herein.

In particular embodiment the ABP of the invention is an antibody having a heavy chain CDR3 amino acid sequence and/or having a light chain CDR3 amino acid sequence, and preferably having a combination of heavy chain CDR1, CDR2 and CDR3 amino acid sequences and/or of and light chain CDR1, CDR2 and CDR3 amino acid sequences, as shown in Table 13.1A for an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007, in each case independently, optionally with no more than eight, seven, six, five or four (eg, for L-CDR3), or no more than three or two, preferably no more than one amino acid substitution(s) insertion(s) or deletion(s) (in particular, substitution(s)) compared to these sequences), or an antigen binding fragment or variant thereof. In another and/or further particular embodiment, the ABP is an antibody having a variable heavy chain amino acid sequence and/or a variable light chain amino acid sequence as shown in Table 13.1A for an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007, in each case independently, optionally with no more than fifteen, fourteen, thirteen, twelve or eleven (eg, for variable light chain), or no more than about 20, 18, 16, 14 or 12, or no more than ten, nine, eight, seven, six, five, four, three, two or one, preferably no more than three, two or one amino acid substitution(s) insertion(s) or deletion(s) (in particular, substitution(s)) compared to these sequences), or an antigen binding fragment or variant thereof.

In one alternative embodiment, the ABP of the invention does not inhibit the interaction between the VSIR (VISTA) protein or a variant thereof and the IgC2 domain of (or the IgV domain of) IGSF11 (VSIG3) protein or a variant thereof, such as described in more details above,

ABPs Comprising One or More Complementarity Determining Regions, One or More of which ABPs May be, Preferentially, Excluded from the Invention

In particular embodiments, an ABP of the invention can preferentially not be one or more ABP (or herein also referred to as an ABP (preferentially) excluded from the invention) that comprise(s) at least one complementarity determining region (CDR) from an antibody (in particular from a human antibody), and having an amino acid sequence set forth in Table 1A herein, or with at least 80%, 85%, 90% or 95% sequence identity to (preferably, at least 90% sequence identity to), or having no more than five or four (eg, for L-CDR1), such as having no more than three or two, preferably no more than one amino acid substitution(s), deletion(s) or insertion(s) (in particular, substitution(s)) compared to, a CDR sequence set forth in Table 1A herein.

The term “ABP (preferably) excluded from the invention” or a grammatically similar expression, in the context of any of the herein disclosed aspects and/or embodiments of the invention that in any way relate to, in connection with or otherwise involve or refer to ABPs, including ABPs per-se, nucleic acids encoding ABPs (or components thereof), methods involving a use or production of an ABP, or any uses of such ABPs (or such nucleic acids), can be understood to mean that an ABP is preferred with the proviso that such ABP is not an ABP referred to herein as an ABP (preferably) excluded from the invention.

As described above, in particular embodiments of the invention, an ABP (preferentially) excluded from the invention can comprise at least one complementarity determining region (CDR). In certain of such embodiments, an ABP (preferentially) excluded from the invention comprises at least one complementarity determining region 3 (CDR3), such as one having an amino acid sequence with at least 80%, 85%, 90% or 95% (preferably at least 90%) sequence identity to, or having no more than five or four, such as having no more than three or two, preferably no more than one amino acid substitution(s), deletion(s) or insertion(s) (in particular, substitution(s)) compared to, a sequence selected from those heavy and light chain CDR3 sequences shown in Table 1A (eg, a sequence selected from the list consisting of SEQ ID Nos: 3, 7, 13, 17, 23, 27, 33, 37, 43, 47, 53, 57, 63, 67, 73, 77, 83, 87, 93, 97, 103, 107, 113, 117, 123, 127, 133, 137, 143, 147, 153, 157, 163, 167, 173, 177, 183, 187, 193, 197, 203, 207, 213, 217, 223, 227, 233, 237, 243, 247, 253, 257, 263, 267, 273, 277, 283, 287, 293, 297, 303, 307, 313, 317, 323, 327, 333, 337, 343, 347, 353, 357, 363, and 367; or in particular eg an amino acid sequence of a CDR3 as shown in Table 1A for the corresponding heavy chain or light chain CDR3 of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or as shown in Table 1A for the corresponding heavy chain or light chain CDR3 of antibody A-024).

An ABP (preferentially) excluded from the invention may, alternatively or as well as a CDR3 sequence, comprise at least one CDR1, and/or at least one CDR2 (such as one from an antibody, in particular from a human antibody). Preferably, an ABP (preferentially) excluded from the invention comprises at least one such CDR3, as well as at least one such CDR1 and at least one such CDR2, more preferably where each of such CDRs having an amino acid sequence with at least 80%, 85%, 90% or 95% (preferably at least 90%) sequence identity to, or having no more than five or four (eg, for L-CDR1), such as having no more than three or two, preferably no more than one amino acid substitution(s), deletion(s) or insertion(s) (in particular, substitution(s)) compared to, a sequence selected from the corresponding (heavy and light chain) CDR1, CDR2 and CDR3 sequences shown in Table 1A (eg compared to an amino acid sequence of a CDR1, CDR2 and/or CDR3 sequence of the corresponding (heavy and light chain) CDR1, CDR2 and CDR3 sequences as shown in Table 1A for an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or as shown in Table 1A for the corresponding heavy chain or light chain CDR3 of antibody A-024).

In particular embodiments, an ABP (preferentially) excluded from the invention can be an antibody or an antigen binding fragment thereof.

In some embodiments of the herein disclosed invention, the ABPs that preferably do not form part of the invention are defined by sequence similarity to CDR and/or variable domain regions of the specific examples of antibodies discovered herein, namely antibodies A-001 to A-037 or B-001 to B-008. Particularly preferred excluded ABPs are ABPs of such embodiments where compared to the herein disclosed sequence, the corresponding sequence defining the ABP (preferentially) excluded from the invention comprises one or more amino acid substitution(s), deletion(s) or insertion(s) (in particular, substitution(s)); for example: (i) the CDR sequence defining an ABP (preferentially) excluded from the invention may have at least 80%, 85%, 90% or 95% (preferably at least 90%) sequence identity to, or may have no more than five or four, such as may have no more than three or two, preferably no more than one amino acid substitution(s), deletion(s) or insertion(s) (in particular, substitution(s)) compared to, the corresponding CDR sequence disclosed herein; and/or (ii) the variable chain sequence defining an ABP (preferentially) excluded from the invention may have at least 80%, 85%, 90%; or 95% (preferably at least 90%) sequence identity to, or may have no more than fifteen, fourteen, thirteen, twelve or eleven (eg, for variable light chain), such as may have no more than ten, nine, eight, seven, six, five, four, three, two or one, preferably no more than three, two or one amino acid substitution(s), deletion(s) or insertion(s) (in particular, substitution(s)) compared to, the corresponding variable chain sequence disclosed herein, in each case independently, optionally a conservative amino acid substitution. In these embodiments, the following is specifically preferred. A CDR3 sequence of an ABP (preferentially) excluded from the invention in preferred embodiments may vary by no more than one amino acid substitution(s), deletion(s) or insertion(s) (in particular, substitution(s)) compared to a sequence selected from the corresponding (preferably light chain) CDR3 sequences shown in Table 1A (in particular, of a CDR3 sequence of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or of antibody A-024), and/or is located not more than 3 amino acid positions away from the CDR3 C-terminus; and/or is a conservative amino acid substitution; and/or is an amino acid substitution from said CDR3 sequence, most preferably is a substitution from a to d or d to a. Alternatively or additionally, a CDR2 sequence of an ABP (preferentially) excluded from the invention in preferred embodiments may vary by no more than one amino acid substitution(s), deletion(s) or insertion(s) (in particular, substitution(s)) compared to a sequence selected from the corresponding (preferably light chain) CDR2 sequences shown in Table 1A (in particular, of a CDR2 sequence of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or of antibody A-024), and/or is located not more than 2 amino acid positions away from the CDR2 C-terminus; and/or is a conservative amino acid substitution; and/or is an amino acid substitution from said CDR2 sequence, most preferably is a substitution from h to d or d to h. Alternatively or additionally, a CDR1 sequence of an ABP (preferentially) excluded from the invention in preferred embodiments may vary by no more than four, preferably no more than three, amino acid substitution(s), deletion(s) or insertion(s) (in particular, substitution(s)) compared to a sequence selected from the corresponding (preferably light chain) CDR1 sequences shown in Table 1A (in particular, of a CDR2 sequence of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or of antibody A-024), and/or is located not more than 5 amino acid positions away from the CDR1 C-terminus; and/or is located at the CDR1 N-terminus; and/or is a conservative amino acid substitution; and/or is an amino acid substitution from said CDR1 sequence, most preferably is a substitution between residues g to a, a to g, n to y, y to n, l to y and/or y to l. Alternatively or additionally, a variable region sequence of an ABP (preferentially) excluded from the invention in preferred embodiments may vary by no more than 13 amino acid substitution(s), deletion(s) or insertion(s) (in particular, substitution(s)) (eg, in each case independently, optionally a conservative amino acid substitution) compared to a sequence selected from the corresponding (preferably light chain) variable sequences shown in Table 1A (in particular, of a variable region sequence of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or of antibody A-024), preferably, wherein independently of the above said for CDR1 to CDR3, no more than seven amino acid substitution(s), deletion(s) or insertion(s) (in particular, substitution(s)) located in the variable region framework, and/or in the case of an antibody heavy chain variable region not more than two amino acid substitution(s), deletion(s) or insertion(s) (in particular, substitution(s)) located in the FRI region.

Accordingly, in certain embodiments an ABP (preferentially) excluded from the invention can comprise an antibody heavy chain, or an antigen binding fragment thereof, and/or an antibody light chain, or an antigen binding fragment thereof.

In further embodiments, an ABP (preferentially) excluded from the invention can comprise an antibody heavy chain variable region, or an antigen binding fragment thereof, and/or an antibody light chain variable region, or an antigen binding fragment thereof, and in yet further embodiments, an ABP (preferentially) excluded from the invention can comprise an antibody heavy chain variable region CDR1, CDR2, and CDR3, and/or an antibody light chain variable region CDR1, CDR2, and CDR3.

In particular embodiments of the invention, when the ABP excluded (preferentially) from the invention comprises an antibody heavy chain sequence and/or an antibody light chain sequence, or an antigen binding fragment thereof; the antibody heavy chain sequence, or the fragment thereof, can comprise a CDR3 having at least 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a CDR3 sequence selected from those heavy chain CDR3 sequences shown in Table 1A (eg, a sequence selected from the list consisting of SEQ ID Nos: 3, 13, 23, 33, 43, 53, 63, 73, 83, 93, 103, 113, 123, 133, 143, 153, 163, 173, 183, 193, 203, 213, 223, 233, 243, 253, 263, 273, 283, 293, 303, 313, 323, 333, 343, 353, and 363; or in particular eg an amino acid sequence of a heavy chain CDR3 as shown in Table 1A for the corresponding heavy chain CDR3 of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or as shown in Table 1A for the corresponding heavy chain CDR3 of antibody A-024), and/or wherein antibody light chain sequence, or the fragment thereof, can comprise a CDR3 having at least 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a CDR3 sequence selected from those light chain CDR3 sequences shown in Table 1A (eg, a sequence selected from the list consisting of SEQ ID Nos: 7, 17, 27, 37, 47, 57, 67, 77, 87, 97, 107, 117, 127, 137, 147, 157, 167, 177, 187, 197, 207, 217, 227, 237, 247, 257, 267, 277, 287, 297, 307, 317, 327, 337, 347, 357, and 367; or in particular eg an amino acid sequence of a light chain CDR3 as shown in Table 1A for the corresponding light chain CDR3 of an antibody selected from any one of the antibodies of the group consisting of: A-002, A-005, A-015, A-006, A-007, A-011, A-012, A-024, A-026, A-027, A-013, A-022, and A-035, preferably antibody A-006, A-012 or A-022 (such as A-006 or A-012), or as shown in Table 1A for the corresponding light chain CDR3 of antibody A-024).

In further embodiments of the invention, when the ABP (preferentially) excluded from the invention comprises an antibody heavy chain, or an antigen binding fragment thereof, the antibody heavy chain sequence, or the fragment thereof, can further comprise a CDR1 having at least 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a sequence selected from SEQ ID NOs. 1, 11, 21, 31, 41, 51, 61, 71, 81, 91, 101, 111, 121, 131, 141, 151, 161, 171, 181, 191, 201, 211, 221, 231, 241, 251, 261, 271, 281, 291, 301, 311, 321, 331, 341, 351, and 361 (eg a heavy chain CDR1 sequence disclosed in Table 1A; or in particular eg an amino acid sequence of a heavy chain CDR1 as shown in Table 1A for the corresponding heavy chain CDR1 of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or as shown in Table 1A for the corresponding heavy chain CDR1 of antibody A-024); and/or a CDR2 having at 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a sequence selected from SEQ ID NOs. 2, 12, 22, 32, 42, 52, 62, 72, 82, 92, 102, 112, 122, 132, 142, 152, 162, 172, 182, 192, 202, 212, 222, 232, 242, 252, 262, 272, 282, 292, 302, 312, 322, 332, 342, 352, and 362 (eg a CDR2 sequence disclosed in Table 1A; or in particular eg an amino acid sequence of a heavy chain CDR2 as shown in Table 1A for the corresponding heavy chain CDR2 of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or as shown in Table 1A for the corresponding heavy chain CDR2 of antibody A-024).

In yet further embodiments of the present invention, an ABP (preferentially) excluded from the invention comprises an antibody light chain, or an antigen binding fragment thereof, wherein the antibody light chain sequence, or the fragment thereof, further comprises a CDR1 having at least 80%, 85%, 90%; or 95% (preferably at least 90%) sequence identity to, or having no more than five or four (eg, for L-CDR1), such as having no more than three or two, preferably no more than one amino acid substitution(s), deletion(s) or insertion(s) (in particular, substitution(s)) compared to, a sequence selected from SEQ ID NOs. 5, 15, 25, 35, 45, 55, 65, 75, 85, 95, 105, 115, 125, 135, 145, 155, 165, 175, 185, 195, 205, 215, 225, 235, 245, 255, 265, 275, 285, 295, 305, 315, 325, 335, 345, 355, and 365 361 (eg a light chain CDR1 sequence disclosed in Table 1A; or in particular compared to eg an amino acid sequence of a light chain CDR1 as shown in Table 1A for the corresponding light chain CDR1 of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or as shown in Table 1A for the corresponding light chain CDR1 of antibody A-024); and/or a CDR2 having at least 80%, 85%, 90%; or 95% (preferably at least 90%) sequence identity to, or having no more than five or four, such as having no more than three or two, preferably no more than one amino acid substitution(s), deletion(s) or insertion(s) (in particular, substitution(s)) compared to, a sequence selected from SEQ ID NOs. 6, 16, 26, 36, 46, 56, 66, 76, 86, 96, 106, 116, 126, 136, 146, 156, 166, 176, 186, 196, 206, 216, 226, 236, 246, 256, 266, 276, 286, 296, 306, 316, 326, 336, 346, 356, and 366 (eg a light chain CDR2 sequence disclosed in Table 1A; or in particular eg compared to an amino acid sequence of a light chain CDR2 as shown in Table 1A for the corresponding light chain CDR2 of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or as shown in Table 1A for the corresponding light chain CDR2 of antibody A-024).

In other embodiments of the present invention, an ABP (preferentially) excluded from the invention can comprise an antibody variable chain sequence having at least 80%, 85%, 90%; or 95% (preferably at least 90%) sequence identity to, or having no more than fifteen, fourteen, thirteen, twelve or eleven (eg, for variable light chain), such as having no more than ten, nine, eight, seven, six, five, four, three, two or one, preferably no more than three, two or one amino acid substitution(s), deletion(s) or insertion(s) (in particular, substitution(s)) compared to, a sequence selected from SEQ ID NOs. 4, 8, 14, 18, 24, 28, 34, 38, 44, 48, 54, 58, 64, 68, 74, 78, 84, 88, 94, 98, 104, 108, 114, 118, 124, 128, 134, 138, 144, 148, 154, 158, 164, 168, 174, 178, 184, 188, 194, 198, 204, 208, 214, 218, 224, 228, 234, 238, 244, 248, 254, 258, 264, 268, 274, 278, 284, 288, 294, 298, 304, 308, 314, 318, 324, 328, 334, 338, 344, 348, 354, 358, 364, and 368 (eg, a VH or VL sequence disclosed in Table 1A; or in particular eg compared to an amino acid sequence of an antibody variable chain sequence as shown in Table 1A for the corresponding heavy or light variable chain of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or as shown in Table 1A for the corresponding heavy or light variable chain of antibody A-024).

In particular embodiments of the invention, an ABP (preferentially) excluded by the invention comprises an antigen binding fragment of an antibody, wherein the antigen binding fragment comprises CDR1, CDR2 and CDR3. In certain of such embodiments, the CDR1 is selected from those disclosed in Table 1A, the CDR2 is selected from those disclosed in Table 1A and the CDR3 is selected from those disclosed in Table 1A (eg, the CDR1, CDR2 and CDR3 are selected from the CDR1, CDR2 and CDR3 sequences having the respective amino acid sequences of SEQ ID Nos. 1, 2, 3, or 5, 6, 7, or 11, 12, 13, or 15, 16, 17, or 21, 22, 23, or 25, 26, 27, or 31, 32, 33, or 35, 36, 37, or 41, 42, 43, or 45, 46, 47, or 51, 52, 53, or 55, 56, 57, or 61, 62, 63, or 65, 66, 67, or 71, 72, 73, or 75, 76, 77, or 81, 82, 83, or 85, 86, 87, or 91, 92, 93, or 95, 96, 97, or 101, 102, 103, or 105, 106, 107, or 111, 112, 113, or 115, 116, 117, or 121, 122, 123, or 125, 126, 127, or 131, 132, 133, or 135, 136, 137, or 141, 142, 143, or 145, 146, 147, or 151, 152, 153, or 155, 156, 157, or 161, 162, 163, or 165, 166, 167, or 171, 172, 173, or 175, 176, 177, or 181, 182, 183, or 185, 186, 187, or 191, 192, 193, or 195, 196, 197, or 201, 202, 203, or 205, 206, 207, or 211, 212, 213, or 215, 216, 217, or 221, 222, 223, or 225, 226, 227, or 231, 232, 233, or 235, 236, 237, or 241, 242, 243, or 245, 246, 247, or 251, 252, 253, or 255, 256, 257, or 261, 262, 263, or 265, 266, 267, or 271, 272, 273, or 275, 276, 277, or 281, 282, 283, or 285, 286, 287, or 291, 292, 293, or 295, 296, 297, or 301, 302, 303, or 305, 306, 307, or 311, 312, 313, or 315, 316, 317, or 321, 322, 323, or 325, 326, 327, or 331, 332, 333, or 335, 336, 337, or 341, 342, 343, or 345, 346, 347, or 351, 352, 353, or 355, 356, 357, or 361, 362, 363, or 365, 366, 367; or in particular eg are amino acid sequences of a CDR1, CDR2 and CDR3 sequence and/or a CDR1, CDR2 and CDR3 sequence as shown in Table 1A for the corresponding CDR1, CDR2 and CDR3 of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or as shown in Table 1A for the corresponding CDR1, CDR2 and CDR3 of antibody A-024); in each case independently, optionally with no more than five or four (eg, for L-CDR1), or with no more than three or two, preferably no more than one, amino acid substitution(s), insertion(s) or deletion(s) (in particular, substitution(s)) compared to these sequences.

In further particular embodiments of the present invention, an ABP (preferentially) excluded by the invention can comprise an antibody heavy chain variable region CDR1, CDR2, and CDR3, and/or an antibody light chain variable region CDR1, CDR2, and CDR3, wherein the CDR1 has an amino acid sequence of a heavy or light chain CDR1 shown in Table 1A (eg has an amino acid sequence selected from the list consisting of SEQ ID No 1, 5, 11, 15, 21, 25, 31, 35, 41, 45, 51, 55, 61, 65, 71, 75, 81, 85, 91, 95, 101, 105, 111, 115, 121, 125, 131, 135, 141, 145, 151, 155, 161, 165, 171, 175, 181, 185, 191, 195, 201, 205, 211, 215, 221, 225, 231, 235, 241, 245, 251, 255, 261, 265, 271, 275, 281, 285, 291, 295, 301, 305, 311, 315, 321, 325, 331, 335, 341, 345, 351, 355, 361, and 365; or in particular eg has an amino acid sequence of an antibody heavy or light chain variable region CDR1 sequence as shown in Table 1A for the corresponding heavy or light chain CDR1 of an antibody selected from any one of the antibodies of the group consisting of: A-002, A-005, A-015, A-006, A-007, A-011, A-012, AA-026, A-027, A-013, A-022, and A-035, preferably antibody A-006, A-012 or A-022 (such as A-006 or A-012), or as shown in Table 1A for the corresponding heavy or light chain CDR1 of antibody A-024), and wherein the CDR2 has an amino acid sequence of a heavy or light chain CDR2 shown in Table 1A (eg has an amino acid sequence selected from the list consisting of SEQ ID No 2, 6, 12, 16, 22, 26, 32, 36, 42, 46, 52, 56, 62, 66, 72, 76, 82, 86, 92, 96, 102, 106, 112, 116, 122, 126, 132, 136, 142, 146, 152, 156, 162, 166, 172, 176, 182, 186, 192, 196, 202, 206, 212, 216, 222, 226, 232, 236, 242, 246, 252, 256, 262, 266, 272, 276, 282, 286, 292, 296, 302, 306, 312, 316, 322, 326, 332, 336, 342, 346, 352, 356, 362, and 366; or in particular eg has an amino acid sequence of an antibody heavy or light chain variable region CDR2 sequence as shown in Table 1A for the corresponding heavy or light chain CDR2 of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or as shown in Table 1A for the corresponding heavy or light chain CDR2 of antibody A-024), and wherein the CDR3 has an amino acid sequence of a heavy or light chain CDR3 shown in Table 1A (eg has an amino acid sequence selected from the list consisting of SEQ ID No 3, 7, 13, 17, 23, 27, 33, 37, 43, 47, 53, 57, 63, 67, 73, 77, 83, 87, 93, 97, 103, 107, 113, 117, 123, 127, 133, 137, 143, 147, 153, 157, 163, 167, 173, 177, 183, 187, 193, 197, 203, 207, 213, 217, 223, 227, 233, 237, 243, 247, 253, 257, 263, 267, 273, 277, 283, 287, 293, 297, 303, 307, 313, 317, 323, 327, 333, 337, 343, 347, 353, 357, 363, and 367; or in particular eg has an amino acid sequence of an antibody heavy or light chain variable region CDR3 sequence as shown in Table 1A for the corresponding heavy or light chain CDR3 of an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), or as shown in Table 1A for the corresponding heavy or light chain CDR3 of antibody A-024); in each case independently, optionally with no more than five or four (eg, for L-CDR1), or with no more than three or two, preferably no more than one, amino acid substitution(s), insertion(s) or deletion(s) (in particular, substitution(s)) compared to these sequences.

In preferred of such embodiments, the ABP (preferentially) excluded from the invention may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise CDR1 to CDR3 sequences in a combination selected from any of the combinations of heavy chain CDRs shown in Table B and or Table B.1 and/or selected from any of the combinations of light chain CDRs shown in Table B (in each case, combinations CDRs-A-001 to CDRs-A-037) and/or selected from any of the combinations of light chain CDRs shown in in Table B.1 (in each case, combinations CDRs-B-001 to CDRs-B-008); in each case independently, optionally with no more than five or four (eg, for L-CDR1), or with no more than three or two, preferably no more than one, amino acid substitution(s), insertion(s) or deletion(s) (in particular, substitution(s)) compared to these sequences. Preferably, the combination of both the heavy chain CDRs and the light chain CDRs is one selected from a row marked by any one of the combinations CDRs-A-001 to CDRs-A-037, or is one selected from a row marked by any one of the combinations and CDRs-B-001 to CDRs-B-008, in each CDR independently optionally with no more than five or four (eg, for L-CDR1), or with no more than three or two, preferably no more than one, amino acid substitution(s), insertion(s) or deletion(s) (in particular, substitution(s)) compared to these sequences.

TABLE B
preferred combinations of heavy chain CDRs and preferred
combinations of light chain CDRs of ABPs (preferentially)
excluded from the invention

| | Heavy Chain | | | Light Chain | | |
|---|---|---|---|---|---|---|
| Combination | CDR1 to CDR3 | | | CDR1 to CDR3 | | |
| (ID) | (SEQ ID NO) | | | (SEQ ID NO) | | |
| CDRs-A-001 | 1 | 2 | 3 | 5 | 6 | 7 |
| CDRs-A-002 | 11 | 12 | 13 | 15 | 16 | 17 |
| CDRs-A-003 | 21 | 22 | 23 | 25 | 26 | 27 |
| CDRs-A-004 | 31 | 32 | 33 | 35 | 36 | 37 |
| CDRs-A-005 | 41 | 42 | 43 | 45 | 46 | 47 |
| CDRs-A-006 | 51 | 52 | 53 | 55 | 56 | 57 |
| CDRs-A-007 | 61 | 62 | 63 | 65 | 66 | 67 |
| CDRs-A-008 | 71 | 72 | 73 | 75 | 76 | 77 |
| CDRs-A-009 | 81 | 82 | 83 | 85 | 86 | 87 |
| CDRs-A-010 | 91 | 92 | 93 | 95 | 96 | 97 |
| CDRs-A-011 | 101 | 102 | 103 | 105 | 106 | 107 |
| CDRs-A-012 | 111 | 112 | 113 | 115 | 116 | 117 |
| CDRs-A-013 | 121 | 122 | 123 | 125 | 126 | 127 |
| CDRs-A-014 | 131 | 132 | 133 | 135 | 136 | 137 |
| CDRs-A-015 | 141 | 142 | 143 | 145 | 146 | 147 |
| CDRs-A-016 | 151 | 152 | 153 | 155 | 156 | 157 |
| CDRs-A-017 | 161 | 162 | 163 | 165 | 166 | 167 |
| CDRs-A-018 | 171 | 172 | 173 | 175 | 176 | 177 |
| CDRs-A-019 | 181 | 182 | 183 | 185 | 186 | 187 |
| CDRs-A-020 | 191 | 192 | 193 | 195 | 196 | 197 |
| CDRs-A-021 | 201 | 202 | 203 | 205 | 206 | 207 |
| CDRs-A-022 | 211 | 212 | 213 | 215 | 216 | 217 |
| CDRs-A-023 | 221 | 222 | 223 | 225 | 226 | 227 |
| CDRs-A-024 | 231 | 232 | 233 | 235 | 236 | 237 |
| CDRs-A-025 | 241 | 242 | 243 | 245 | 246 | 247 |
| CDRs-A-026 | 251 | 252 | 253 | 255 | 256 | 257 |
| CDRs-A-027 | 261 | 262 | 263 | 265 | 266 | 267 |
| CDRs-A-028 | 271 | 272 | 273 | 275 | 276 | 277 |
| CDRs-A-029 | 281 | 282 | 283 | 285 | 286 | 287 |
| CDRs-A-030 | 291 | 292 | 293 | 295 | 296 | 297 |
| CDRs-A-031 | 301 | 302 | 303 | 305 | 306 | 307 |
| CDRs-A-032 | 311 | 312 | 313 | 315 | 316 | 317 |
| CDRs-A-033 | 321 | 322 | 323 | 325 | 326 | 327 |
| CDRs-A-034 | 331 | 332 | 333 | 335 | 336 | 337 |
| CDRs-A-035 | 341 | 342 | 343 | 345 | 346 | 347 |
| CDRs-A-036 | 351 | 352 | 353 | 355 | 356 | 357 |
| CDRs-A-037 | 361 | 362 | 363 | 365 | 366 | 367 |

TABLE B.1
further preferred combinations of heavy chain CDRs and preferred
combinations of light chain CDRs of ABPs (preferentially)
excluded from the invention

| | | | | | | |
|---|---|---|---|---|---|---|
| CDRs-B-001 | 111 | 112 | 113 | 125 | 126 | 127 |
| CDRs-B-002 | 51 | 52 | 53 | 115 | 116 | 117 |
| CDRs-B-003 | 111 | 112 | 113 | 45 | 46 | 47 |
| CDRs-B-004 | 111 | 112 | 113 | 55 | 56 | 57 |
| CDRs-B-005 | 111 | 112 | 113 | 15 | 16 | 17 |
| CDRs-B-006 | 51 | 52 | 53 | 45 | 46 | 47 |
| CDRs-B-007 | 51 | 52 | 53 | 125 | 126 | 127 |
| CDRs-B-008 | 51 | 52 | 53 | 15 | 16 | 17 |

In other preferred embodiments of the invention, the ABP (preferentially) excluded from the invention may be an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown in Table C and/or Table C.1 (eg, selected from any of the variable chain combinations Chains-A-001 to Chains-A-037, or selected from any of the variable chain combinations Chains-B-001 to Chains-B-008); in each case independently, optionally with no more than fifteen, fourteen, thirteen, twelve or eleven (eg, for variable light chain), such with no more than ten, nine, eight, seven, six, five, four, preferably no more than three, two or one, amino acid substitution(s), insertion(s) or deletion(s) (in particular, substitution(s)) compared to these sequences.

TABLE C

| preferred | | |
|---|---|---|
| combinations of | | |
| heavy and light | | |
| chain variably | | |
| domains of ABPs | | |
| (preferentially) | | |
| excluded from the | Heavy Chain | Light Chain |
| invention | Variable Domain | Variable Domain |
| Combination (ID) | (SEQ ID NO) | (SEQ ID NO) |
| Chains-A-001 | 4 | 8 |
| Chains-A-002 | 14 | 18 |
| Chains-A-003 | 24 | 28 |
| Chains-A-004 | 34 | 38 |
| Chains-A-005 | 44 | 48 |
| Chains-A-006 | 54 | 58 |
| Chains-A-007 | 64 | 68 |
| Chains-A-008 | 74 | 78 |
| Chains-A-009 | 84 | 88 |
| Chains-A-010 | 94 | 98 |
| Chains-A-011 | 104 | 108 |
| Chains-A-012 | 114 | 118 |
| Chains-A-013 | 124 | 128 |
| Chains-A-014 | 134 | 138 |
| Chains-A-015 | 144 | 148 |
| Chains-A-016 | 154 | 158 |
| Chains-A-017 | 164 | 168 |
| Chains-A-018 | 174 | 178 |
| Chains-A-019 | 184 | 188 |
| Chains-A-020 | 194 | 198 |
| Chains-A-021 | 204 | 208 |
| Chains-A-022 | 214 | 218 |
| Chains-A-023 | 224 | 228 |
| Chains-A-024 | 234 | 238 |
| Chains-A-025 | 244 | 248 |
| Chains-A-026 | 254 | 258 |
| Chains-A-027 | 264 | 268 |
| Chains-A-028 | 274 | 278 |
| Chains-A-029 | 284 | 288 |
| Chains-A-030 | 294 | 298 |
| Chains-A-031 | 304 | 308 |
| Chains-A-032 | 314 | 318 |
| Chains-A-033 | 324 | 328 |
| Chains-A-034 | 334 | 338 |
| Chains-A-035 | 344 | 348 |
| Chains-A-036 | 354 | 358 |
| Chains-A-037 | 364 | 368 |

TABLE C.1
further preferred combinations of heavy and light chain variably
domains of ABPs (preferentially) excluded from the invention

| | | |
|---|---|---|
| Chains-B-001 | 114 | 128 |
| Chains-B-002 | 54 | 118 |
| Chains-B-003 | 114 | 48 |
| Chains-B-004 | 114 | 58 |
| Chains-B-005 | 114 | 18 |
| Chains-B-006 | 54 | 48 |
| Chains-B-007 | 54 | 128 |
| Chains-B-008 | 54 | 18 |

In preferred of such embodiments, the ABP (preferentially) excluded from the invention may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences comprise CDR1 to CDR3 sequences selected from the sequences shown in SEQ ID NO: 51, 52 and 53; or 111, 112, and 113; or 211, 212 and 213; or 231, 232 and 233; and at least one, preferably both, of the antibody light chain sequences comprise CDR1 to CDR3 sequences in a combination selected from any of the combinations of light chain CDRs shown in Table B; in each case independently, optionally with no more than three or two, preferably no more than one, amino acid substitution(s), insertion(s) or deletion(s) (in particular, substitution(s)) compared to these sequences. Most preferably (preferentially) excluded from the invention is a combination indicated for rows CDRs-A006, CDRs-A-012 or CDRs-A-022; or row CDRs-A-024.

In preferred of such embodiments, the ABP (preferentially) excluded from the invention may be an antibody, or an antigen binding fragment thereof, composed of at least one, preferably two, antibody heavy chain sequences, and at least one, preferably two, antibody light chain sequences, wherein at least one, preferably both, of the antibody light chain sequences comprise CDR1 to CDR3 sequences selected from the sequences shown in SEQ ID NO: 55, 56 and 57; or 115, 116, and 117; or 125, 126 and 127; or 45, 46 and 47; or 15, 16 and 17; or 235, 236 and 237; and at least one, preferably both, of the antibody heavy chain sequences comprise CDR1 to CDR3 sequences in a combination selected from any of the combinations of heavy chain CDRs shown in Table B; in each case independently, optionally with no more than five or four (eg for L-CDR1), such as no more than three or two, preferably no more than one, amino acid substitution(s), insertion(s) or deletion(s) (in particular, substitution(s)) compared to these sequences.

In preferred of such embodiments, the ABP (preferentially) excluded from the invention may be an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise CDR1 to CDR3 sequences in the combination of the combinations of heavy and light chain CDRs shown in Table B rows: CDRs-A-002, CDRs-A-005, CDRs-A-015, CDRs-A-006, CDRs-A-007, CDRs-A-011, CDRs-A-012, CDRs-A-026, CDRs-A-027, CDRs-A-013, CDRs-A-022, or CDRs-A-035; or CDRs-A-024; in each case independently, optionally with no more than three or two, preferably no more than one, amino acid substitution(s), insertion(s) or deletion(s) (in particular, substitution(s)) compared to these sequences.

In other preferred embodiments of the invention, the ABP (preferentially) excluded from the invention may be an antibody, or an antigen binding fragment thereof, composed of at least one, preferably two, antibody heavy chain sequence, and at least one, preferably two, antibody light chain sequence, wherein the at least one, preferably two, antibody heavy chain sequence comprises a variable region sequence selected from the sequences according to SEQ ID NO: 54, 114 or 214; or according to SEQ ID NO 234; and wherein the least one, preferably two, antibody light chain sequence comprises a light chain variable domain shown in Table C; in each case independently, optionally with no more than fifteen, fourteen, thirteen, twelve or eleven (eg, for variable light chain), or with no more than ten, nine, eight, seven, six, five, four, preferably no more than three, two or one, amino acid substitution(s), insertion(s) or deletion(s) (in particular, substitution(s)) compared to these sequences.

In other preferred embodiments of the invention, the ABP (preferentially) excluded from the invention may be an antibody, or an antigen binding fragment thereof, composed of at least one, preferably two, antibody heavy chain sequence, and at least one, preferably two, antibody light chain sequence, wherein the at least one, preferably two, antibody light chain sequence comprises a variable region sequence selected from the sequences according to SEQ ID NO: 18, 48, 58, 118, 128, or 218; or according to SEQ ID NO 238; and wherein the least one, preferably two, antibody heavy chain sequence comprises a heavy chain variable domain shown in Table C; in each case independently, optionally with no more than fifteen, fourteen, thirteen, twelve or eleven (eg, for variable light chain), or with no more than ten, nine, eight, seven, six, five, four, preferably no more than three, two or one, amino acid substitution(s), insertion(s) or deletion(s) (in particular, substitution(s)) compared to these sequences.

In other preferred embodiments of the invention, the ABP (preferentially) excluded from the invention may be an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown in Table C rows Chains-A-002, Chains-A-005, Chains-A-015, Chains-A-006, Chains-A-007, Chains-A-011, Chains-A-012, Chains-A-026, Chains-A-027, Chains-A-013, Chains-A-022, or Chains-A-035; or in row Chains-A-024; in each case independently, optionally with no more than fifteen, fourteen, thirteen, twelve or eleven (eg, for variable light chain), or with no more than ten, nine, eight, seven, six, five, four, preferably no more than three, two or one, amino acid substitution(s), insertion(s) or deletion(s) (in particular, substitution(s)) compared to these sequences.

In other preferred embodiments of the invention, the ABP (preferentially) excluded from the invention may be an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown in Table C-1 rows Chains-B-001 to Chains-B-008, in particular in row Chains-B-001 or Chains-B-002; in each case independently, optionally with no more than fifteen, fourteen, thirteen, twelve or eleven (eg, for variable light chain), or with no more than ten, nine, eight, seven, six, five, four, preferably no more than three, two or one, amino acid substitution(s), insertion(s) or deletion(s) (in particular, substitution(s)) compared to these sequences.

In particularly preferred embodiment, an ABP (preferentially) excluded from the invention can comprise a combination of heavy chain CDR1, CDR2 and CDR3 sequences and a combination of light chain CDR1, CDR2 and CDR3 sequences in the combination shown by antibody A-015, such as shown in Table B by row CDRs-A-015 (eg, heavy chain CDR1, CDR2 and CDR3 having a sequence shown by SEQ ID Nos, 141, 142 and 143, respectively, and light chain CDR1, CDR2 and CDR3 having a sequence shown by SEQ ID Nos, 145, 146 and 147, respectively), in each CDR independently, optionally with no more than five or four (eg for L-CDR1), such as with no more than three or two, preferably no more than one, amino acid substitution(s), insertion(s) or deletion(s) (in particular, substitution(s)) compared to these sequences. In another particularly preferred embodiment, an ABP (preferentially) excluded from the invention can be an antibody, or an antigen binding fragment thereof, composed of at least one, preferably two, antibody heavy chain sequences, and at least one, preferably two, antibody light chain sequences, wherein at least one, preferably both, of the antibody heavy chain sequences each comprises heavy chain CDR1 to CDR3 sequences in the combination CDRs-A-015 and at least one, preferably both, of the antibody light chain sequences each comprises light chain CDR1 to CDR3 sequences in the combination shown in the row of Table B marked by CDRs-A-015, in each CDR independently, optionally with no more than one amino acid substitution(s), insertion(s) or deletion(s) (in particular, substitution(s)) compared to these sequences. In yet another particularly preferred embodiment, an ABP (preferentially) excluded from the invention can be an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown the row of Table B marked by Chains-A-015. In each of such particularly preferred embodiments of the ABP (preferentially) excluded from the invention, optionally, the ABP (preferentially) excluded from the invention is able to inhibit the binding of VSIR protein or a variant thereof to IGSF11 protein or a variant thereof with an IC50 of 20 nM or less or 10 nM or less, such as 5 nM or less, or preferably 2 nM or less. Such IC50s can be determined using the methods described elsewhere herein.

In a particular embodiment the ABP (preferentially) excluded from the invention can be an antibody having a heavy chain CDR3 amino acid sequence and/or having a light chain CDR3 amino acid sequence, and preferably having a combination of heavy chain CDR1, CDR2 and CDR3 amino acid sequences and/or of and light chain CDR1, CDR2 and CDR3 amino acid sequences, as shown in Table 1A for an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), (in each case independently, optionally with no more than five or four (eg, for L-CDR1), or no more than three or two, preferably no more than one amino acid substitution(s) insertion(s) or deletion(s) (in particular, substitution(s)) compared to these sequences), or an antigen binding fragment or variant thereof. In another and/or further particular embodiment, the ABP (preferentially) excluded from the invention is an antibody having a variable heavy chain amino acid sequence and/or a variable light chain amino acid sequence as shown in Table 1A for an antibody selected from any one of the antibodies of the group consisting of: A-002, A-005, A-015, A-006, A-007, A-011, A-012, A-026, A-027, A-013, A-022, and A-035, preferably antibody A-006, A-012 or A-022 (such as A-006 or A-012), (in each case independently, optionally with no more than fifteen, fourteen, thirteen, twelve or eleven (eg, for variable light chain), or no more than ten, nine, eight, seven, six, five, four, three, two or one, preferably no more than three, two or one amino acid substitution(s) insertion(s) or deletion(s) (in particular, substitution(s)) compared to these sequences), or an antigen binding fragment or variant thereof.

In another embodiment the ABP (preferentially) excluded from the invention is an antibody having a combination of heavy chain CDR1, CDR2 and CDR3 amino acid sequences and/or of and light chain CDR1, CDR2 and CDR3 amino acid sequences, as shown in Table 1A for an antibody selected from the group consisting of: B-001, B-002, B-003, B-004, B-005, B-006, B-007 and B-008, and in particular for B-001 or B-002, (in each case independently, optionally with no more than five or four (eg, for L-CDR1), or no more than three or two, preferably no more than one amino acid substitution(s) insertion(s) or deletion(s) (in particular, substitution(s)) compared to these sequences), or an antigen binding fragment or variant thereof. In another and/or further particular embodiment, the ABP (preferentially) excluded from the invention is an antibody having a combination of a variable heavy chain amino acid sequence and a variable light chain amino acid sequence as shown in Table 1A for an antibody selected from the group consisting of: B-001, B-002, B-003, B-004, B-005, B-006, B-007 and B-008, and in particular for B-001 or B-002, (in each case independently, optionally with no more than fifteen, fourteen, thirteen, twelve or eleven (eg, for variable light chain), or no more than ten, nine, eight, seven, six, five, four, three, two or one, preferably no more than three, two or one amino acid substitution(s) insertion(s) or deletion(s) (in particular, substitution(s)) compared to these sequences), or an antigen binding fragment or variant thereof.

In one alternative embodiment, the ABP (preferentially) excluded from the invention does not inhibit the interaction between the VSIR (VISTA) protein or a variant thereof and the IGSF11 (VSIG3) protein or a variant thereof, such as described in more details above. In another particular (and optionally related) embodiment, the ABP (preferentially) excluded from the invention is an antibody having a heavy chain CDR3 amino acid sequence and/or having a light chain amino acid CDR3 sequence, and preferably having a combination of heavy chain CDR1, CDR2 and CDR3 amino acid sequences and/or of and light chain CDR1, CDR2 and CDR3 amino acid sequences, as shown in Table 1A for antibody A-024, (in each case independently, optionally with no more than five or four (eg, for L-CDR1), or no more than three or two, preferably no more than one amino acid substitution(s) insertion(s) or deletion(s) (in particular, substitution(s)) compared to these sequences), or an antigen binding fragment or variant thereof. In another and/or further particular embodiment, the ABP (preferentially) excluded from the invention is an antibody having a variable heavy chain amino acid sequence and/or a variable light chain amino acid sequence as shown in Table 1A for antibody A-024, (in each case independently, optionally with no more than fifteen, fourteen, thirteen, twelve or eleven (eg, for variable light chain), or no more than ten, nine, eight, seven, six, five, four, three, two or one, preferably no more than three, two or one amino acid substitution(s) insertion(s) or deletion(s) (in particular, substitution(s)) compared to these sequences), or an antigen binding fragment or variant thereof.

In an alternative (or additional) example embodiment, the ABP of the invention is (preferentially) not an ABP that is one or more of an antibody that, for example binds to IGSF11 protein, eg binds to the IgC2 domain of IGSF11 (or, in the alternative aspect, that for example binds to the IgV domain of IGSF11) and is selected from the list consisting of antibodies disclosed in WO 2018/027042 A1 (for example, as the heavy chain amino acid sequences of such antibodies are disclosed in FIG. 20B of WO 2018/027042 A1, the light chain amino acid sequences of such antibodies are disclosed in FIG. 20A of WO 2018/027042 A1, the heavy chain CDR amino acid sequences of such antibodies are disclosed in FIG. 18B of WO 2018/027042 A1, the light chain CDR amino acid sequences of such antibodies are disclosed in FIG. 18A of WO 2018/027042 A1, and as summarised in Table D).

TABLE D
SEQ ID NOs of WO 2018/027042 A1 of anti-IGSF11 antibodies
disclosed therein
Antibody ID and SEQ ID NO. of WO 2018/027042 A1

| | Heavy | Light | H- | H- | H- | L- | L- | L- |
|---|---|---|---|---|---|---|---|---|
| Antibody | chain | chain | CDR1 | CDR2 | CDR3 | CDR1 | CDR2 | CDR3 |
| #774206 | 90 | 85 | 40 | 51 | 62 | 7 | 18 | 29 |
| #774208 | 91 | 86 | 41 | 52 | 63 | 8 | 19 | 30 |
| #774213 | 92 | 87 | 42 | 53 | 64 | 9 | 20 | 31 |
| #774221 | 93 | 88 | 43 | 54 | 65 | 10 | 21 | 32 |
| #774226 | 94 | 89 | 44 | 55 | 66 | 11 | 22 | 33 |
| #973401 | 79 | 73 | 45 | 56 | 67 | 12 | 23 | 34 |
| #973408 | 80 | 74 | 46 | 57 | 68 | 13 | 24 | 35 |
| #973422 | 81 | 75 | 47 | 58 | 69 | 14 | 25 | 36 |
| #973428 | 82 | 76 | 48 | 59 | 70 | 15 | 26 | 37 |
| #973433 | 83 | 77 | 49 | 60 | 71 | 16 | 27 | 38 |
| #973435 | 84 | 78 | 50 | 61 | 72 | 17 | 28 | 39 |

In a further alternative (or additional) example embodiment, the ABP of the invention is (preferentially) not an ABP that is one or more of an antibody that, for example binds to IGSF11 protein, eg binds to the IgC2 domain of IGSF11 (or, in the alternative aspect, that for example binds to the IgV domain of IGSF11) and is selected from the list consisting of antibodies disclosed in WO2019/152810A1 (for example, the monoclonal antibodies of WO2019/152810A1 set forth in Table 2A of WO2019/152810A1, or the polyclonal antibodies set forth in Table 3A of WO2019/152810A1) In particular of such embodiments, the ABP is not a (mouse or rat, as applicable) monoclonal antibody produced from an antibody clone identified in WO2019/152810A1 as those in the list consisting of: 973404, 973422, 973423, 973436, 973435, 993501, 993502, 993508, 993512, 993515, 993518, 993521, 993527, 993611, 993619, 993620, 993622, 993625, 993626, 993628, 993630, 993820, 993821, 993822, 993826, 993836, 993839, 993843, 993848 and 993851. In another particular of such embodiments, the ABP is not a (rabbit) polyclonal antibody produced from an antibody clone identified in WO2019/152810A1 as those in the list consisting of: Q111, H89, L138, 1205, V216, Y176, G129, C44, 5154, D194, G78, C120, Q33, N66, C165 and K186.

Further Aspects and Embodiments of ABPs of the Invention, in Particular Biological/Biochemical Function(s) Thereof

In a second aspect, the invention relates to an ABP which competes with an ABP of a first aspect for binding to a C2-type immunoglobulin-like (IgC2) domain of IGSF11 protein (or to an IgV domain of IGSF11 protein) or variant thereof, in particular can relate to an ABP that competes with one of the particularly preferred ABPs described above for binding to a IgC2 domain of the IGSF11 protein or variant (or to a IgV domain of the IGSF11 protein or variant).

The term “compete” when used in the context of ABPs (e.g., modulator ABPs) that compete for binding for the same antigen (or epitope displayed by such antigen) means competition between ABPs as may be determined by an assay in which the ABP (e.g., antibody or binding fragment thereof) being tested prevents or inhibits (e.g., reduces) binding of a reference ABP (e.g., a ligand, or a reference antibody) to a common antigen (e.g., IGSF11 or a fragment thereof such as an ECD of IGSF11, and in particular to an IgC2 domain of IGSF11).

In a related aspect, the invention relates to an ABP which binds to the same epitope as an ABP of a first aspect.

In certain embodiments of this second (or related) aspect, the ABP of this aspect (ie, which competes for binding with, and/or binds to the same epitope as, an ABP of the first aspect), is not one or more of any of the ABPs that are comprised in the provisos of the first aspect. For example, in one of such embodiments the ABP of this this second (or related) aspect is not an ABP that is one or more of: (A) one or more of an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown selected from any of the variable chain combinations Chains-A-001 to Chains-A-037 (as shown in Table C); and (B) one or more of an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown selected from any of the variable chain combinations Chains-B-001 to Chains-B-008 (as shown in Table C.1).

In an alternative (or additional) example embodiment, the ABP of this second (or related) aspect is (preferentially) not an ABP that is one or more of an antibody that, for example binds to the IgC2 domain of IGSF11 (or, in the alternative aspect, that for example binds to the IgV domain of IGSF11) and is selected from the list consisting of antibodies: #774206, #774208, #774213, #774221, #774226, #973401, #973408, #973422, #973428, #973433 and #973435, each as disclosed in WO 2018/027042 A1 (for example, as the heavy chain amino acid sequences of such antibodies are disclosed in FIG. 20B of WO 2018/027042 A1, the light chain amino acid sequences of such antibodies are disclosed in FIG. 20A of WO 2018/027042 A1, the heavy chain CDR amino acid sequences of such antibodies are disclosed in FIG. 18B of WO 2018/027042 A1, the light chain CDR amino acid sequences of such antibodies are disclosed in FIG. 18A of WO 2018/027042 A1, and as described in Table D).

ABPs of a second aspect of the invention may include one or more features (or specific combinations thereof) of the ABPs described above. In particular, an ABP of a second aspect of the invention may be capable of inhibiting (eg inhibits) the binding of an interacting protein (eg, VSIR (VISTA) protein or a variant thereof) to IGSF11 (VSIG3) protein or to an IgC2 domain of IGSF11 protein (or, in the other aspect, to an IgV domain of IGSF11 protein), or a variant thereof, such as described in more details above, and/or an ABP of a second aspect of the invention may modulate the expression, function, activity and/or stability of IGSF11 or such domain of IGSF11, or the variant thereof (such as in anyway described elsewhere herein).

In particular embodiments of the invention, as well as (or instead of) an ABP of the invention's capability to inhibit (eg block) the interaction between an interacting protein (eg, VSIR (VISTA) protein or a variant thereof) to IGSF11 (VSIG3) protein or to an IgC2 domain of IGSF11 protein (or, in the other aspect, to an IgV domain of IGSF11 protein), or a variant thereof, an ABP of the invention (including those of a first or second aspect as above) may display, exhibit or otherwise possess other functional features, in particular those which are associated with their utility in sensitising cells to a cell-mediated immune response.

In certain of such particular embodiments, an ABP of the invention is capable of reducing (eg it reduces) the amount and/or surface concentration of said IGSF11, or of an IgC2 domain of IGSF11 protein (or, in the other aspect, of an IgV domain of IGSF11 protein), or the variant thereof present on the surface of a mammalian cell; preferably by ABP-induced internalisation, and optionally degradation, of said IGSF11 (of said domain) or the variant thereof present on the surface of the mammalian cell.

In further of such particular embodiments, an ABP of the invention is capable of enhancing (eg it enhances) killing and/or lysis of cells expressing IGSF11, or a variant of IGSF11, by cytotoxic T cells and/or TILs. Such enhancement can be assessed, for example, using a suitable assay such as one described in Comparative Example 7 hereof.

A particular functional characteristic of an ABP of the invention may be that of increasing (eg, an IBP of the invention increases) the activity of immune cells, such as T-cells, including when such T-cells recognise a mammalian cell expressing the IGSF11 or the variant of IGSF11, or are are bound to the surface of a mammalian cell expressing said IGSF11 or the variant of IGSF11. An increase in eg T cells may be an increase in production of a pro-inflammatory cytokine such as IL-2 (such as may be measured as described in Comparative Examples 8 and/or 9).

The term “immune cell” is art recognised to describe any cell of an organism involved in the immune system of such organism, in particular of a mammal such as a human. Leukocytes (white blood cells) are immune cells that are involved in the innate immune system, and the cells of the adaptive immune system are special types of leukocytes, known as lymphocytes. B cells and T cells are the major types of lymphocytes and are derived from hematopoietic stem cells in the bone marrow. B cells are involved in the humoral immune response, whereas T cells are involved in cell-mediated immune response. In preferred embodiments of the invention, the immune cell can be a myeloid cell eg a T cell, and in particular (such as when an increase in cell-mediated immune response is required, such as to treat a cancer) the T cell can be a cytotoxic T cell (also known as TC, cytotoxic T lymphocyte, CTL, T-killer cell, cytolytic T cell, CD8+ T-cell or killer T cell). A CTL is a T-cell that is involved in the killing of cancer cells, cells that are infected (particularly with viruses), or cells that are damaged in other ways. Other preferred immune cells for such embodiments can include Tumour-Infiltrating Lymphocytes (TILs). TILs are white blood cells that have left the bloodstream and migrated into a tumour. Typically, TILs are a mix of different types of cells (i.e., T cells, B cells, NK cells) in variable proportions, T cells being the most abundant cells. TILs can often be found in the stroma and within the tumour itself, and are implicated in killing tumour cells. The presence of lymphocytes in tumours is often associated with better clinical outcomes.

Other particular functional characteristics of an ABP of the invention may be that of: (i) enhancing a cell-mediated immune response, such as that mediated by an activated cytotoxic T-cell (CTL), to a mammalian cell expressing said IGSF11 (or said domain) or the variant thereof; and/or (ii) increasing immune cell, such as T-cell, activity and/or survival (and/or proliferation) in the presence of a mammalian cell expressing said IGSF11 (or said domain) or the variant thereof. In some embodiments, the mammalian cell expressing the IGSF11 (or the domain) may be a cell associated with a disease, disorder or condition such as a cancer cell being (directly) associated with the cancer. In other the mammalian cell expressing the IGSF11 (or the domain) may be an immune cell, such as a T cell (see below), for example an immune cell that is directly or indirectly associated with the disease, disorder or condition.

Other particular functional characteristics of an ABP of the invention that is an inhibitor or antagonist of IGSF11 expression, function, activity and/or stability, or of the expression, function, activity and/or stability of an IgC2 (or of an IgV) domain of IGSF11, can be any one, or a combination or at least one, functional characteristic of the inhibiting or antagonistic modulators described herein, in particular in the section above “Modulators of IGSF11 expression, function, activity and/or stability”.

Those particular functional characteristics of an ABP of the invention that is an activator or agonist of IGSF11 expression, function, activity and/or stability, or of the expression, function, activity and/or stability of an IgC2 (or of an IgV) domain of IGSF11, can be any one, or a combination or at least one, functional characteristic of the activating or agonistic modulators described herein, in particular in the section above “Modulators of IGSF11 expression, function, activity and/or stability”.

In preferred embodiments of all ABPs of the invention, the ABP is isolated and/or substantially pure.

The term “isolated” as used herein in the context of a protein, such as an ABP (an example of which could be an antibody), refers to a protein that is purified from proteins or polypeptides or other contaminants that would interfere with its therapeutic, diagnostic, prophylactic, research or other use. An isolated ABP according to the invention may be a recombinant, synthetic or modified (non-natural) ABP. The term “isolated” as used herein in the context of a nucleic acid or cells refers to a nucleic acid or cells that is/are purified from DNA, RNA, proteins or polypeptides or other contaminants (such as other cells) that would interfere with its therapeutic, diagnostic, prophylactic, research or other use, or it refers to a recombinant, synthetic or modified (non-natural) nucleic acid. Preferably an isolated ABP or nucleic acid or cells is/are substantially pure. In this context, a “recombinant” protein or nucleic acid is one made using recombinant techniques. Methods and techniques for the production of recombinant nucleic acids and proteins are well known in the art.

The term “isolated” as used herein in the context of a protein, such as an ABP (an example of which could be an antibody), refers to a protein that is purified from proteins or polypeptides or other contaminants that would interfere with its therapeutic, diagnostic, prophylactic, research or other use. An isolated ABP according to the invention may be a recombinant, synthetic or modified (non-natural) ABP. The term “isolated” as used herein in the context of a nucleic acid or cells refers to a nucleic acid or cells that is/are purified from DNA, RNA, proteins or polypeptides or other contaminants (such as other cells) that would interfere with its therapeutic, diagnostic, prophylactic, research or other use, or it refers to a recombinant, synthetic or modified (non-natural) nucleic acid. Preferably an isolated ABP or nucleic acid or cells is/are substantially pure. In this context, a “recombinant” protein or nucleic acid is one made using recombinant techniques. Methods and techniques for the production of recombinant nucleic acids and proteins are well known in the art.

In some embodiments, an ABP of the invention may bind to (e.g., via one or more epitope(s) displayed by one or more EC domain(s) of) IGSF11 or a paralogue, orthologue or other variant thereof (such as any IGSF11 or variant described herein), or in particular, may bind to an IgC2 domain of IGSF11 (or, in the other aspects, may bind to an IgV domain of IGSF11) with a KD that is less than 20 nM, such as less than about 10 nM, 5 nM or 2 nM (in particular, less than about 1 nM). In a preferred embodiment, the ABP of the invention will bind (e.g. said epitope(s) of) said IGSF11 or said domain, or variant thereof, with a KD that is less than 100 pM. In a more preferred embodiment, the ABP of the invention will bind said IGSF11 or said domain, or variant thereof, with a KD that is less than 10 pM. In a most preferred embodiment, the ABP of the invention will bind said IGSF11 or said domain, or variant thereof, with a KD that is less than 2 pM. Binding of an ABP of the invention, such as an antibody of the invention, to a human cell line expressing said IGSF11 or said domain, or variant thereof, may, in some embodiments, occur at an EC50 of less than about 10 μg/mL, 5 μg/mL, 2 μg/mL, 1 μg/mL, 0.5 μg/mL or 0.2 μg/mL, preferably with an EC50 of less than 2 μg/mL. Binding of an ABP of the invention, such as an antibody of the invention, to a Cynomolgus cell line expressing an orthologue of said IGSF11 or said domain, or variant thereof, may, in some embodiments, occur at an EC50 of less than about 10 μg/mL, 5 μg/mL, 2 μg/mL, 1 μg/mL, 0.5 μg/mL or 0.2 μg/mL, preferably with an EC50 of less than 2 μg/mL.

In other embodiments, an ABP of the invention may: (i) bind to the IGSF11 or the (eg, IgC2) domain of IGSF11, or to the variant thereof, with a KD that is less than 20 nM, such as less than about 10 nM, 5 nM or 2 nM (in particular, less than about 1 nM), is less than 100 pM, or is less than 10 pM; and/or (ii) binds to a human cell line expressing the IGSF11 or the domain of IGSF11, or the variant thereof, with an EC50 of less than 2 ug/mL.

In certain preferred embodiments, the ABP of the invention, in particular those shown in table 13.3 below, as well as their respective ABP variants, and most preferably D-114, D-115, D-116, D-222 and/or D-223, are characterized by having unexpectedly strong affinity to their target, an IgC2 domain of IGSF11. Hence, in preferred embodiments such ABPs disclosed herein, or their respective variants, have an affinity KD of less than 150 pM, more preferably of less than 100 pM, and in certain cases as is disclosed in table 14.1 herein for D-114, an affinity that is characterized by a KD of less than 10 pM, or even below detectability. Such affinity is preferably measurable using using a kinetic exclusion assay.

The term “KD”, as used herein, is intended to refer to the dissociation constant, which is obtained from the ratio of Kd to Ka (i. e., Kd/Ka) and is expressed as a molar concentration (M). KD values for antibodies can be determined using methods well established in the art such as plasmon resonance (BIAcore®), ELISA and KINEXA. A preferred method for determining the KD of an antibody is by using surface plasmon resonance, preferably using a biosensor system such as a BIAcore® system or by ELISA. “Ka” (or “K-assoc”), as used herein, refers broadly to the association rate of a particular antibody-antigen interaction, whereas the term “Kd” (or “K-diss”), as used herein, refers to the dissociation rate of a particular antibody-antigen interaction.

In one embodiment, an ABP of the invention specifically binds to the IgC2 domain of IGSF11 (such as to the IgC2 domain of human, mouse and/or cynomolgus monkey IGSF11), and binds to such IgC2 domain with an (eg, apparent) affinity that is less than about 200 nM or 150 nM, such as less than about 125 nM, 75 nm or 50 nm, and suitably with an (eg, apparent) affinity that is less than about 25 nM or 15 nM (such as less than about 10 nM or 5 nM). Such an ABP of the invention will, typical, not substantially, appreciably or detectably bind to the IgV domain of such IGSF11.

In an alternative embodiment, an ABP of the invention specifically binds to the IgV domain of IGSF11 (such as to the IgV domain of human, mouse and/or cynomolgus monkey IGSF11), and binds to such IgV domain with an (eg, apparent) affinity that is less than about 500 nM, 250 nM or 150 nM, such as less than about 125 nM, 75 nm or 50 nm, and suitably with an (eg, apparent) affinity that is less than about 25 nM or 15 nM (such as less than about 10 nM or 5 nM). Such an ABP of the invention will, typical, not substantially, appreciably or detectably bind to the IgC2 domain of such IGSF11.

In yet other embodiments, an ABP of the invention may compete for binding to IGSF11, or to the variant of IGSF11, or to an IgC2 domain of IGSF11 protein (or, in the other aspect, to an IgV domain of IGSF11 protein), with an interacting protein, such as an endogenous IGSF11 ligand or receptor or partner, preferably wherein said interacting protein endogenous IGSF11 ligand or receptor is VSIR, or a variant of VSIR. For example, in certain of such embodiments, the ABP of the invention (eg one that binds to [one or more epitope(s) displayed by] an extracellular domain(s) of IGSF11 (such as an IgC2 domain of (or IgV domain of) IGSF11, or a paralogue, orthologue or other variant thereof) is capable of inhibiting (eg will inhibit) the binding of the interacting protein, such as VSIR protein or a variant thereof to IGSF11 protein or domain of IGSF11, or a variant thereof, with an IC50 of 100 nM, 50 nM, or preferably 20 nM or less, such as 15 nM or less, 10 nM or less, 5 nM or less, 2 nM or less, 1 nM or less, 500 pM or less, 250 pM or less, or 100 pM or less. In particular of such embodiments, an ABP of the invention is capable of inhibiting (eg will inhibit) the binding of interacting protein, such as VSIR protein or a variant thereof to IGSF11 protein or domain of IGSF11, or a variant thereof, with an IC50 of 10 nM or less, such as 5 nM or less and preferably 2 nM or less.

Exemplary Types of ABPs, their Identification/Generation/Discovery and Modification

In one embodiment, an ABP of the invention is a polyclonal antibody (mixture), or the antigen binding fragment is a fragment of a polyclonal antibody (mixture).

In another embodiment, an ABP of the invention is not a polyclonal antibody, or the antigen binding fragment is not a fragment of a polyclonal antibody. In more specific embodiments, an ABP of the invention is not an anti-IGSF11 polyclonal sheep IgG (or, is not antibody number AF4915 from R&D Systems), and/or is not an anti-IGSF11 polyclonal rabbit IgG (or, is not antibody number orb1928 from biorbyt and/or is not antibody number MBP1-59503 from Novus Biologicals).

In an alternative, and preferred, embodiment of all ABPs of the invention, the ABP is an antibody or an antigen binding fragment thereof, and the antibody is a monoclonal antibody, or wherein the antigen binding fragment is a fragment of a monoclonal antibody.

The term “monoclonal antibody” or “mAb” as used herein refers to an antibody obtained from a population of substantially identical antibodies based on their amino acid sequence. Monoclonal antibodies are typically highly specific. Furthermore, in contrast to conventional (polyclonal) antibody preparations which typically include different antibodies directed against different determinants (e.g. epitopes) of an antigen, each mAb is typically directed against a single determinant on the antigen. In addition to their specificity, mAbs are advantageous in that they can be synthesized by cell culture (hybridomas, recombinant cells or the like) uncontaminated by other immunoglobulins. The mAbs herein include for example chimeric, humanized or human antibodies or antibody fragments.

Monoclonal antibodies in accordance with the present invention may be prepared by methods well known to those skilled in the art. For example, mice, rats, goats, camels, alpacas, llamas or rabbits may be immunized with an antigen of interest (or a nucleic acid encoding an antigen of interest) together with adjuvant. Splenocytes are harvested as a pool from the animals that are administered several immunisations at certain intervals with test bleeds performed to assess for serum antibody titers. Splenocytes are prepared that are either used immediately in fusion experiments or stored in liquid nitrogen for use in future fusions. Fusion experiments are then performed according to the procedure of Stewart & Fuller, J. Immunol. Methods 1989, 123:45-53. Supernatants from wells with growing hybrids are screened by eg enzyme-linked immunosorbent assay (ELISA) for mAb secretors. ELISA-positive cultures are cloned either by limiting dilutions or fluorescence-activated cell sorting, typically resulting in hybridomas established from single colonies. The ability of an antibody, including an antibody fragment or sub-fragment, to bind to a specific antigen can be determined by binding assays known in the art, for example, using the antigen of interest as the binding partner.

Antibodies in accordance with the present invention may be prepared by genetic immunisation methods in which native proteins are expressed in vivo with normal post-transcriptional modifications, avoiding antigen isolation or synthesis. For example, hydrodynamic tail or limb vein delivery of naked plasmid DNA expression vectors can be used to produce the antigen of interest in vivo in mice, rats, and rabbits and thereby induce antigen-specific antibodies (Tang et al, Nature 356: 152 (1992); Tighe et al, Immunol. Today 19: 89 (1998); Bates et al, Biotechniques, 40:199 (2006); Aldevron-Genovac, Freiburg DE). This allows the efficient generation of high-titre, antigen-specific antibodies which may be particularly useful for diagnostic and/or research purposes. For such genetic immunisation, a variety of gene delivery methods can be used, including direct injection of naked plasmid DNA into skeletal muscle, lymph nodes, or the dermis, electroporation, ballistic (gene gun) delivery, and viral vector delivery.

In a further preferred embodiment, an ABP of the invention is an antibody or an antigen binding fragment thereof, wherein the antibody is a human antibody a humanised antibody or a chimeric-human antibody, or wherein the antigen binding fragment is a fragment of a human antibody a humanised antibody or a chimeric-human antibody.

Human antibodies can also be derived by in vitro methods. Suitable examples include but are not limited to phage display (CAT, Morphosys, Dyax, Biosite/Medarex, Xoma, Yumab, Symphogen, Alexion, Affimed) and the like. In phage display, a polynucleotide encoding a single Fab or Fv antibody fragment is expressed on the surface of a phage particle (see e.g., Hoogenboom et al., J. Mol. Biol., 227: 381 (1991); Marks et al., J Mol Biol 222: 581 (1991); U.S. Pat. No. 5,885,793). Phage are “screened” to identify those antibody fragments having affinity for target. Thus, certain such processes mimic immune selection through the display of antibody fragment repertoires on the surface of filamentous bacteriophage, and subsequent selection of phage by their binding to target. In certain such procedures, high affinity functional neutralizing antibody fragments are isolated. A complete repertoire of human antibody genes may thus be created by cloning naturally rearranged human V genes from peripheral blood lymphocytes (see, e.g., Mullinax et al., Proc Natl Acad Sci (USA), 87: 8095-8099 (1990)) or by generating fully synthetic or semi-synthetic phage display libraries with human antibody sequences (see Knappik et al 2000; J Mol Biol 296:57; de Kruif et al, 1995; J Mol Biol 248):97).

The antibodies described herein may alternatively be prepared through the utilization of the XenoMouse® technology. Such mice are capable of producing human immunoglobulin molecules and antibodies and are deficient in the production of murine immunoglobulin molecules and antibodies. In particular, a preferred embodiment of transgenic production of mice and antibodies is disclosed in U.S. patent application Ser. No. 08/759,620, filed Dec. 3, 1996 and International Patent Application Nos. WO 98/24893, published Jun. 11, 1998 and WO 00/76310, published Dec. 21, 2000. See also Mendez et al., Nature Genetics, 15:146-156 (1997). Through the use of such technology, fully human monoclonal antibodies to a variety of antigens have been produced. Essentially, XenoMouse® lines of mice are immunized with an antigen of interest. e.g. IGSF11 (VSIG3), lymphatic cells (such as B-cells) are recovered from the hyper-immunized mice, and the recovered lymphocytes are fused with a myeloid-type cell line to prepare immortal hybridoma cell lines. These hybridoma cell lines are screened and selected to identify hybridoma cell lines that produce antibodies specific to the antigen of interest. Other “humanised” mice are also commercially available: eg, Medarex—HuMab mouse, Kymab—Kymouse, Regeneron—Velocimmune mouse, Kirin—TC mouse, Trianni—Trianni mouse, OmniAb—OmniMouse, Harbour Antibodies—H2L2 mouse, Merus—MeMo mouse. Also are available are “humanised” other species: rats: OmniAb—OmniRat, OMT—UniRat. Chicken: OmniAb—OmniChicken.

The term “humanised antibody” according to the present invention refers to immunoglobulin chains or fragments thereof (such as Fab, Fab′, F(ab′)2, Fv, or other antigen-binding sub-sequences of antibodies), which contain minimal sequence (but typically, still at least a portion) derived from non-human immunoglobulin. For the most part, humanised antibodies are human immunoglobulins (the recipient antibody) in which CDR residues of the recipient antibody are replaced by CDR residues from a non-human species immunoglobulin (the donor antibody) such as a mouse, rat or rabbit having the desired specificity, affinity and capacity. As such, at least a portion of the framework sequence of said antibody or fragment thereof may be a human consensus framework sequence. In some instances, Fv framework residues of the human immunoglobulin need to be replaced by the corresponding non-human residues to increase specificity or affinity. Furthermore, humanised antibodies can comprise residues which are found neither in the recipient antibody nor in the imported CDR or framework sequences. These modifications are made to further refine and maximise antibody performance. In general, the humanised antibody will comprise substantially all of at least one, and typically at least two, variable domains, in which all or substantially all of the CDR regions correspond to those of a non-human immunoglobulin and all or substantially all of the framework regions are those of a human immunoglobulin consensus sequence. The humanised antibody optimally also will comprise at least a portion of an immunoglobulin constant region, typically that of a human immunoglobulin, which (eg human) immunoglobulin constant region may be modified (eg by mutations or glycoengineering) to optimise one or more properties of such region and/or to improve the function of the (eg therapeutic) antibody, such as to increase or reduce Fc effector functions or to increase serum half-life. Exemplary such Fc modification (for example, Fc engineering or Fc enhancement) are described elsewhere herein.

The term “chimeric antibody” according to the present invention refers to an antibody whose light and/or heavy chain genes have been constructed, typically by genetic engineering, from immunoglobulin variable and constant regions which are identical to, or homologous to, corresponding sequences of different species, such as mouse and human. Alternatively, variable region genes derive from a particular antibody class or subclass while the remainder of the chain derives from another antibody class or subclass of the same or a different species. It covers also fragments of such antibodies. For example, a typical therapeutic chimeric antibody is a hybrid protein composed of the variable or antigen-binding domain from a mouse antibody and the constant or effector domain from a human antibody, although other mammalian species may be used.

In particular of such embodiments, an ABP of the invention comprises an antigen binding domain of an antibody wherein the antigen binding domain is of a human antibody. Preferably, ABP comprises an antigen binding domain of an antibody or an antigen binding fragment thereof, which is a human antigen binding domain; (ii) the antibody is a monoclonal antibody, or wherein the antigen binding fragment is a fragment of a monoclonal antibody; and (iii) the antibody is a human antibody or a humanised antibody, or wherein the antigen binding fragment is a fragment of a human antibody, a humanised antibody or a chimeric-human antibody.

Light chains of human antibodies generally are classified as kappa and lambda light chains, and each of these contains one variable region and one constant domain. Heavy chains are typically classified as mu, delta, gamma, alpha, or epsilon chains, and these define the antibody's isotype as IgM, IgD, IgG, IgA, and IgE, respectively. Human IgG has several subtypes, including, but not limited to, IgG1, IgG2, IgG3, and IgG4. Human IgM subtypes include IgM, and IgM2. Human IgA subtypes include IgA1 and IgA2. In humans, the IgA and IgD isotypes contain four heavy chains and four light chains; the IgG and IgE isotypes contain two heavy chains and two light chains; and the IgM isotype contains ten or twelve heavy chains and ten or twelve light chains. Antibodies according to the invention may be IgG, IgE, IgD, IgA, or IgM immunoglobulins.

In some embodiments, the ABP of the invention is an IgG antibody or fragment thereof. In some embodiments, the ABP of the invention is an IgE antibody or fragment thereof. In some embodiments, the ABP of the invention is an IgD antibody or fragment thereof. In some embodiments, the ABP of the invention is an IgA antibody or fragment thereof. In some embodiments, the ABP of the invention is an IgM antibody or fragment thereof. Preferably the ABP of the invention is, comprises or is derived from an IgG immunoglobulin or fragment thereof; such as a human, human-derived IgG immunoglobulin, or a rabbit- or rat-derived IgG, and/or an IgG2 immunoglobulin, or fragment thereof. When the ABP of the invention is, comprises or is derived from a rat-derived IgG, then preferably, the ABP is, comprises or is derived from, a rat IgG2a or IgG2b immunoglobulin. When the ABP of the invention is, comprises or is derived from a human-derived IgG, then more preferably, the ABP of the invention is, comprises or is derived from a human IgG1, IgG2 or IgG4, most preferably, the ABP of the invention is, comprises or is derived from a human IgG1 or IgG2.

Accordingly, in particular embodiments of the invention, an ABP is an antibody wherein the antibody is an IgG, IgE, IgD, IgA, or IgM immunoglobulin; preferably an IgG immunoglobulin.

An ABP of the invention, where comprising at least a portion of an immunoglobulin constant region (typically that of a human immunoglobulin) may have such (eg human) immunoglobulin constant region modified—for example eg by glycoengineering or mutations—to optimise one or more properties of such region and/or to improve the function of the (eg therapeutic) antibody, such as to increase or reduce Fc effector functions or to increase serum half-life.

ABPs of the invention, in particular those useful in the present methods include antibodies that induce antibody-dependent cytotoxicity (ADCC) of IGSF11-expressing cells. The ADCC of an anti-IGSF11 antibody can be improved by using antibodies that have low levels of or lack fucose. Antibodies lacking fucose have been correlated with enhanced ADCC (antibody-dependent cellular cytotoxicity) activity, especially at low doses of antibody (Shields et ah, 2002, J. Biol. Chem. 277:26733-26740; Shinkawa et ah, 2003, J. Biol. Chem. 278:3466).

Methods of preparing fucose-less antibodies or antibodies with reduced fucose levels include growth in rat myeloma YB2/0 cells (ATCC CRL 1662). YB 2/0 cells express low levels of FUT8 mRNA, which encodes an enzyme (.alpha. 1,6-fucosyltransferase) necessary for fucosylation of polypeptides.

Alternatively, during the expression of such antibodies, an inhibitor against an enzyme relating to the modification of a sugar chain may be used, including: tunicamycin which selectively inhibits formation of GlcNAc—P—P-Dol which is the first step of the formation of a core oligosaccharide which is a precursor of an N-glycoside-linked sugar chain, castanospermin and W-methyl-1-deoxynojirimycin which are inhibitors of glycosidase I, kifunensine which is an inhibitor of mannosidase I, bromocondulitol which is an inhibitor of glycosidase II, 1-deoxynojirimycin and 1,4-dioxy-1,4-imino-D-mannitol which are inhibitors of mannosidase I, swainsonine which is an inhibitor of mannosidase II and the like. Examples of an inhibitor specific for a glycosyltransferase include deoxy derivatives of substrates against N-acetylglucosamine transferase V (GnTV) and the like. Also, it is known that 1-deoxynojirimycin inhibits synthesis of a complex type sugar chain and increases the ration of high mannose type and hybrid type sugar chains (Glycobiology series 2-Destiny of Sugar Chain in Cell, edited by Katsutaka Nagai, Senichiro Hakomori and Akira Kobata, 1993).

Based on these data, several cell lines have been genetically engineered to produce antibodies containing no or low levels of fucose (Mori et al, 2004; Yamane-Ohnuki et al., 2004) to engineer the glycosylation patterns of IgG in order to select therapeutic monoclonal antibodies exhibiting particular profiles of Fc-gamma-R engagement that could be used in various pathologies.

Umana et al. and Davis et al. showed that an IgG1 antibody engineered to contain increasing amounts of bisected complex oligosaccharides (bisecting A/-acetylglucosamine, GlcNAC) allows triggering a strong ADCC as compared to its parental counterpart (Umana et al., 1999; Davies et al., 2001). Second, a lack of fucose on human IgG1 N-linked oligosaccharides has been shown to improve FCGRIII binding and ADCC.

GLYCART BIOTECHNOLOGY AG (Zurich, CH) has expressed N-acetyl-glucosaminyltransferase III (GnTIII) which catalyses the addition of the bisecting GlcNac residue to the N-linked oligosaccharide, in a Chinese hamster ovary (CHO) cell line, and showed a greater ADCC of IgG1 antibody produced (WO 99/54342; WO 03/01 1878; WO 2005/044859).

WO20070166306 is related to the modification of an antibody anti-CD19 containing 60% N-acetylglucosamine bisecting oligosaccharides and 10% non-fucosylated N-acetylglucosamine bisecting oligosaccharides produced in a mammalian human 293T embryonal kidney cells transfected with (i) the cDNA for the anti-CD19 antibody and (ii) the cDNA for the GnTIII enzyme.

Recombinant human IgG1 produced in YB2/0 cells (Shinkawa et al., 2003; Siberil et al., 2006) or in CHO-Lec13 (Shields et al., 2002) which exhibited a low-fucose content or were deficient in fucose as compared to the same IgG1 produced in wild-type CHO cells, showed an enhanced ability to trigger cellular cytotoxicity. By contrast, a correlation between galactose and ADCC was not observed and the content of bisecting GlcNAC only marginally affected ADCC (Shinkawa et al., 2003).

By removing or supplanting fucose from the Fc portion of the antibody, KYOWA HAKKO KOGYO (Tokyo, Japan) has enhanced Fc binding and improved ADCC, and thus the efficacy of the MAb (U.S. Pat. No. 6,946,292). This improved Fc-gamma-RIIIA-dependent effector functions of low-fucosylated IgG has been shown to be independent from Fc-gamma-RIII allelic form (Niwa et al., 2005). Moreover, it has been recently shown that the antigenic density required to induce an efficient ADCC is lower when the IgG has a low content in fucose as compared to a highly fucosylated IgG (Niwa et al., 2005)

The Laboratoire Francais du Fractionnement et des Biotechnologies (LFB) (France) showed that the ratio Fuc/Gal in MAb oligosaccharide should be equal or lower than 0.6 to get antibodies with a high ADCC (FR 2 861 080).

Cardarelli et al., 2019 produce an anti-CD19 antibody in Ms-704PF CHO cells deficient in the FUT8 gene which encodes alphal-1,6-fucosyltransferase. Non-fucosylation of the antibody in this paper requires the engineering of an enzyme-deficient cell line. This paper does not consider amino acid mutations.

Herbst et al. generated a humanized IgG1 MAb MEDI-551 expressed in a fucosyltransferase-deficient producer CHO cell line. This paper does not consider amino acid mutations (Herbst et al., 2010). Siberil et al used the rat myeloma YB2/0 cell line to produce a MAb anti RhD with a low fucose content. Whereas the MAb produced in a wild type CHO exhibited a high fucose content (81%), the same MAb produced in YB2/0 cell exhibited a lower fucose content (32%). This paper does consider amino acid mutations (Siberil et al., 2006).

Accordingly, an ABP of the invention may be prepared and/or may have one or more of the characteristics of such glycoengineering (eg afucosylated) approaches/antibodies described above.

Alternative methods for increasing ADDC activity for an ABP of the invention include mutations in an Fc portion of such ABP, particularly mutations which increase antibody affinity for an Fc-gamma-R receptor.

Accordingly, any of the ABPs of the invention described above can be produced with different antibody isotypes or mutant isotypes to control the extent of binding to different Fc-gamma receptors. Antibodies lacking an Fc region (e.g., Fab fragments) lack binding to different Fc-gamma receptors. Selection of isotype also affects binding to different Fc-gamma receptors. The respective affinities of various human IgG isotypes for the three different Fc-gamma receptors, Fc-gamma-RI, Fc-gamma-RII, and Fc-gamma-RIII, have been determined. (See Ravetch & Kinet, Annu. Rev. Immunol. 9, 457 (1991)). Fc-gamma-RI is a high affinity receptor that binds to IgGs in monomeric form, and the latter two are low affinity receptors that bind IgGs only in multimeric form. In general, both IgG1 and IgG3 have significant binding activity to all three receptors, IgG4 to Fc-gamma-RI, and IgG2 to only one type of Fc-gamma-RII called IIaLR (see Parren et al., J. Immunol. 148, 695 (1992). Therefore, human isotype IgG1 is usually selected for stronger binding to Fc-gamma receptors, and IgG2 or IgG4 is usually selected for weaker binding.

A correlation between increased Fc-gamma-R binding with mutated Fc has been demonstrated using targeted cytoxicity cell-based assays (Shields et ah, 2001, J. Biol. Chem. 276:6591-6604; Presta et ah, 2002, Biochem Soc. Trans. 30:487-490). Methods for increasing ADCC activity through specific Fc region mutations include the Fc variants comprising at least one amino acid substitution at a position selected from the group consisting of: 234, 235, 239, 240, 241, 243, 244, 245, 247, 262, 263, 264, 265, 266, 267, 269, 296, 297, 298, 299, 313, 325, 327, 328, 329, 330 and 332, wherein the numbering of the residues in the Fc region is that of the EU index as in Kabat (Kabat et ah, Sequences of Proteins of Immunological Interest (National Institute of Health, Bethesda, Md. 1987).

In certain specific embodiments, said Fc variants comprise at least one substitution selected from the group consisting of L234D, L234E, L234N, L234Q, L234T, L234H, L234Y, L234I, L234V, L234F, L235D, L235S, L235N, L235Q, L235T, L235H, L235Y, L235I, L235V, L235F, S239D, S239E, S239N, S239Q, S239F, S239T, S239H, S239Y, V240I, V240A, V240T, V240M, F241W, F241L, F241Y, F241E, F241R, F243W, F243L, F243Y, F243R, F243Q, P244H, P245A, P247V, P247G, V262I, V262A, V262T, V262E, V263I, V263A, V263T, V263M, V264L, V264I, V264W, V264T, V264R, V264F, V264M, V264Y, V264E, D265G, D265N, D265Q, D265Y, D265F, D265V, D265I, D265L, D265H, D265T, V266I, V266A, V266T, V266M, S267Q, S267L, E269H, E269Y, E269F, E269R, Y296E, Y296Q, Y296D, Y296N, Y296S, Y296T, Y296L, Y296I, Y296H, N297S, N297D, N297E, A298H, T299I, T299L, T299A, T299S, T299V, T299H, T299F, T299E, W313F, N325Q, N325L, N325I, N325D, N325E, N325A, N325T, N325V, N325H, A327N, A327L, L328M, L328D, L328E, L328N, L328Q, L328F, L328I, L328V, L328T, L328H, L328A, P329F, A330L, A330Y, A330V, A330I, A330F, A330R, A330H, I332D, 1332E, I332N, I332Q, I332T, I332H, I332Y and I332A, wherein the numbering of the residues in the Fc region is that of the EU index as in Kabat.

Fc variants can also be selected from the group consisting of V264L, V264I, F241W, F241L, F243W, F243L, F241L/F243L/V262I/V264I, F241W/F243W, F241W/F243W/V262A/V264A, F241L/V262I, F243L/V264I, F243L/V262I/V264W, F241Y/F243Y/V262T/V264T, F241E/F243R/V262E/V264R, F241E/F243Q/V262T/V264E, F241R/F243Q/V262T/V264R, F241E/F243Y/V262T/V264R, L328M, L328E, L328F, I332E, L3238M/I332E, P244H, P245A, P247V, W313F, P244H/P245A/P247V, P247G, V264I/I332E, F241E/F243R/V262E/V264R/I332E, F241E/F243Q/V262T/264E/I332E, F241R/F243Q/V262T/V264R/I332E, F241E/F243Y/V262T/V264R/I332E, S298A/I332E, S239E/I332E, S239Q/I332E, S239E, D265G, D265N, S239E/D265G, S239E/D265N, S239E/D265Q, Y296E, Y296Q, T299I, A327N, S267Q/A327S, S267L/A327S, A327L, P329F, A330L, A330Y, I332D, N297S, N297D, N297S/I332E, N297D/I332E, N297E/I332E, D265Y/N297D/I332E, D265Y/N297D/T299L/I332E, D265F/N297E/I332E, L328I/I332E, L328Q/I332E, I332N, I332Q, V264T, V264F, V240I, V263I, V266I, T299A, T299S, T299V, N325Q, N325L, N325I, S239D, S239N, S239F, S239D/I332D, S239D/I332E, S239D/I332N, S239D/I332Q, S239E/I332D, S239E/I332N, S239E/I332Q, S239N/I332D, S239N/I332E, S239N/I332N, S239N/I332Q, S239Q/I332D, S239Q/I332N, S239Q/I332Q, Y296D, Y296N, F241Y/F243Y/V262T/V264T/N297D/I332E, A330Y/I332E, V264I/A330Y/I332E, A330L/I332E, V264I/A330L/I332E, L234D, L234E, L234N, L234Q, L234T, L234H, L234Y, L234I, L234V, L234F, L235D, L235S, L235N, L235Q, L235T, L235H, L235Y, L235I, L235V, L235F, S239T, S239H, S239Y, V240A, V240T, V240M, V263A, V263T, V263M, V264M, V264Y, V266A, V266T, V266M, E269H, E269Y, E269F, E269R, Y296S, Y296T, Y296L, Y296I, A298H, T299H, A330V, A330I, A330F, A330R, A330H, N325D, N325E, N325A, N325T, N325V, N325H, L328D/I332E, L328E/I332E, L328N/I332E, L328Q/I332E, L328V/I332E, L328T/I332E, L328H/I332E, L328I/I332E, L328A, I332T, I332H, I332Y, I332A, S239E/V264I/I332E, S239Q/V264I/I332E, S239E/V264I/A330Y/I332E, S239E/V264I/S298A/A330Y/I332E, S239D/N297D/I332E, S239E/N297D/I332E, S239D/D265V/N297D/I332E, S239D/D265I/N297D/I332E, S239D/D265L/N297D/I332E, S239D/D265F/N297D/I332E, S239D/D265Y/N297D/I332E, S239D/D265H/N297D/I332E, S239D/D265T/N297D/I332E, V264E/N297D/I332E, Y296D/N297D/I332E, Y296E/N297D/I332E, Y296N/N297D/I332E, Y296Q/N297D/I332E, Y296H/N297D/I332E, Y296T/N297D/I332E, N297D/T299V/I332E, N297D/T299I/I332E, N297D/T299L/I332E, N297D/T299F/I332E, N297D/T299H/I332E, N297D/T299E/I332E, N297D/A330Y/I332E, N297D/S298A/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, and S239D/264I/A330L/I332E, wherein the numbering of the residues in the Fc region is that of the EU index as in Kabat. See also WO2004029207, incorporated by reference herein.

In particular embodiments, mutations on, adjacent, or close to sites in the hinge link region (e.g., replacing residues 234, 235, 236 and/or 237 with another residue) can be made, in all of the isotypes, to reduce affinity for Fc-gamma receptors, particularly Fc-gamma-RI receptor (see, eg U.S. Pat. No. 6,624,821). Optionally, positions 234, 236 and/or 237 are substituted with alanine and position 235 with glutamate. (See, eg U.S. Pat. No. 5,624,821.) Position 236 is missing in the human IgG2 isotype. Exemplary segments of amino acids for positions 234, 235 and 237 for human IgG2 are Ala Ala Gly, Val Ala Ala, Ala Ala Ala, Val Glu Ala, and Ala Glu Ala. A preferred combination of mutants is L234A, L235E and G237A, or is L234A, L235A, and G237A for human isotype IgG1. A particular preferred ABP of the invention is an antibody having human isotype IgG1 and one of these three mutations of the Fc region. Other substitutions that decrease binding to Fc-gamma receptors are an E233P mutation (particularly in mouse IgG1) and D265A (particularly in mouse IgG2a). Other examples of mutations and combinations of mutations reducing Fc and/or C1q binding are E318A/K320A/R322A (particularly in mouse IgG1), L235A/E318A/K320A/K322A (particularly in mouse IgG2a). Similarly, residue 241 (Ser) in human IgG4 can be replaced, eg with proline to disrupt Fc binding.

Additional mutations can be made to a constant region to modulate effector activity. For example, mutations can be made to the IgG1 or IgG2 constant region at A330S, P331S, or both. For IgG4, mutations can be made at E233P, F234V and L235A, with G236 deleted, or any combination thereof. IgG4 can also have one or both of the following mutations S228P and L235E. The use of disrupted constant region sequences to modulate effector function is further described, eg in WO2006118,959 and WO2006036291.

Additional mutations can be made to the constant region of human IgG to modulate effector activity (see, e.g., WO200603291). These include the following substitutions: (i) A327G, A330S, P331S; (ii) E233P, L234V, L235A, G236 deleted; (iii) E233P, L234V, L235A; (iv) E233P, L234V, L235A, G236 deleted, A327G, A330S, P331S; and (v) E233P, L234V, L235A, A327G, A330S, P331S to human IgG1; or in particular, (vi) L234A, L235E, G237A, A330S and P331S (eg, to human IgG1), wherein the numbering of the residues in the Fc region is that of the EU index as in Kabat. See also WO2004029207, incorporated by reference herein.

The affinity of an antibody for the Fc-gamma-R can be altered by mutating certain residues of the heavy chain constant region. For example, disruption of the glycosylation site of human IgG1 can reduce Fc-gamma-R binding, and thus effector function, of the antibody (see, eg WO2006036291). The tripeptide sequences NXS and NXT, where X is any amino acid other than proline, are the enzymatic recognition sites for glycosylation of the N residue. Disruption of any of the tripeptide amino acids, particularly in the CH2 region of IgG, will prevent glycosylation at that site. For example, mutation of N297 of human IgG1 prevents glycosylation and reduces Fc-gamma-R binding to the antibody.

Although activation of ADCC and CDC is often desirable for therapeutic antibodies, there are circumstances in which an ABP of the invention unable to activate effector functions is preferential (eg, an ABP of the invention that is an agnostic modulator). For these purposes IgG4 has commonly been used but this has fallen out of favour in recent years due the unique ability of this sub-class to undergo Fab-arm exchange, where heavy chains can be swapped between IgG4 in vivo as well as residual ADCC activity. Accordingly, Fc engineering approaches can also be used to determine the key interaction sites for the Fc domain with Fc-gamma receptors and C1q and then mutate these positions, such as in an Fc of an ABP of the invention, to reduce or abolish binding. Through alanine scanning Duncan and Winter (1998; Nature 332:738) first isolated the binding site of C1q to a region covering the hinge and upper CH2 of the Fc domain. Researchers at Genmab identified mutants K322A, L234A and L235A, which in combination are sufficient to almost completely abolish Fc-gamma-R and C1q binding (Hezareh et al, 2001; J Virol 75:12161). In a similar manner MedImmune later identified a set of three mutations, L234F/L235E/P331S (dubbed TM), which have a very similar effect (Oganesyan et al, 2008; Acta Crystallographica 64:700). An alternative approach is modification of the glycosylation on asparagine 297 of the Fc domain, which is known to be required for optimal FcR interaction. A loss of binding to Fc-gammaRs has been observed in N297 point mutations (Tao et al, 1989; J Immunol 143:2595), enzymatically degylcosylated Fc domains (Mimura et al, 2001; J Biol Chem 276:45539), recombinantly expressed antibodies in the presence of a glycosylation inhibitor (Walker et al, 1989; Biochem J 259:347) and the expression of Fc domains in bacteria (Mazor et al 2007; Nat Biotechnol 25:563). Accordingly, the invention also includes embodiments of the ABPs in which such technologies or mutations have been used to reduce effector functions.

IgG naturally persists for a prolonged period in (eg human) serum due to FcRn-mediated recycling, giving it a typical half-life of approximately 21 days. Despite this there have been a number of efforts to engineer the pH dependant interaction of the Fc domain with FcRn to increase affinity at pH 6.0 while retaining minimal binding at pH 7.4. Researchers at PDL BioPharma identified the mutations T250Q/M428L, which resulted in an approximate 2-fold increase in IgG half-life in rhesus monkeys (Hinto et al, 2004; J Biol Chem 279:6213), and researchers at MedImmune have identified mutations M252Y/S254T/T256E (dubbed YTE), which resulted in an approximate 4-fold increase in IgG half-life in cynomolgus monkeys (Dall'Acqua, et al 2006; J Biol Chem 281:23514). A combination of the M252Y/S254T/T256E mutations with point mutations H433K/N434F lead to similar effects (Vaccaro et al., 2005, Nat Biotechnol. October; 23(10):1283-8). ABPs of the invention may also be PEGylated. PEGylation, ie chemical coupling with the synthetic polymer poly-ethylene glycol (PEG), has emerged as an accepted technology for the development of biologics that exercise prolonged action, with around 10 clinically approved protein and peptide drugs to date (Jevsevar et al., 2010; Biotechnol J 5:113). ABPs of the invention may also be subjected to PASylation, a biological alternative to PEGylation for extending the plasma half-life of pharmaceutically active proteins (Schlapschy et al, 2013; Protein Eng Des Sel 26:489; XL-protein GmbH, Germany). Similarly, the XTEN half-life extension technology from Amunix provides another biological alternative to PEGylation (Schellenberger, 2009, Nat Biotechnol.; 27(12):1186-90. doi: 10.1038/nbt.1588). Accordingly, the invention also includes embodiments of the ABPs in which such technologies or mutations have been used to prolong serum half-life, especially in human serum.

Antibody fragments include “Fab fragments”, which are composed of one constant and one variable domain of each of the heavy and the light chains, held together by the adjacent constant region of the light chain and the first constant domain (CH1) of the heavy chain. These may be formed by protease digestion, e.g. with papain, from conventional antibodies, but similar Fab fragments may also be produced by genetic engineering. Fab fragments include Fab, Fab and “Fab-SH” (which are Fab fragments containing at least one free sulfhydryl group).

Fab′ fragments differ from Fab fragments in that they contain additional residues at the carboxy terminus of the first constant domain of the heavy chain including one or more cysteines from the antibody hinge region. Fab′ fragments include “Fab′-SH” (which are Fab′ fragments containing at least one free sulfhydryl group).

Further, antibody fragments include F(ab′)2 fragments, which contain two light chains and two heavy chains containing a portion of the constant region between the CH1 and CH2 domains (“hinge region”), such that an interchain disulphide bond is formed between the two heavy chains. A F(ab′)2 fragment thus is composed of two Fab′ fragments that are held together by a disulphide bond between the two heavy chains. F(ab′)2 fragments may be prepared from conventional antibodies by proteolytic cleavage with an enzyme that cleaves below the hinge region, e.g. with pepsin, or by genetic engineering.

An “Fv region” comprises the variable regions from both the heavy and light chains, but lacks the constant regions. “Single-chain antibodies” or “scFv” are Fv molecules in which the heavy and light chain variable regions have been connected by a flexible linker to form a single polypeptide chain, which forms an antigen binding region.

An “Fc region” comprises two heavy chain fragments comprising the CH2 and CH3 domains of an antibody. The two heavy chain fragments are held together by two or more disulphide bonds and by hydrophobic interactions of the CH3 domains.

Accordingly, in some embodiments, the ABP of the invention is an antibody fragment selected from the list consisting of: Fab, Fab, Fab′-SH, Fab-SH, Fv, scFv and F(ab′)2.

In those embodiments of ABPs that are fragments of immunoglobulins, such as an antibody fragment, preferred are those fragments capable of binding to (eg an epitope displayed by) the extracellular domain(s) of IGSF11, or a paralogue, orthologue or other variant thereof, such as any epitope or other binding characteristic as described herein: and more preferably said fragment is a modulator (such as an inhibitor or antagonist) of the expression, function, activity and/or stability of IGSF11 or a paralogue, orthologue or other variant of IGSF11.

In a preferred embodiment, an ABP of the invention is an antibody wherein at least a portion of the framework sequence of said antibody or fragment thereof is a human consensus framework sequence, for example, comprises a human germline-encoded framework sequence.

In some embodiments, an ABP of the invention is modified or engineered to increase antibody-dependent cellular cytotoxicity (ADCC). As will now be understood by the person of ordinary skill, such ABPs of the invention will have particular utility in the therapy of diseases or disorders associated with cellular resistance against immune cells like CTLs (such as an IGSF11-positive cancer); as the ADCC mechanism (a cell-mediated immune defence whereby an effector cell of the immune system actively lyses a target cell, whose membrane-surface antigens have been bound by specific antibodies) would be enhanced in respect of the cells having resistance against immune cells like CTLs, hence leading to an increase in attachment by and/or lysis of such cells by effector cells of the immune system.

As used herein, “therapy” is synonymous with treating a disease, disorder or condition, which includes reducing symptoms of the disease, disorder or condition, inhibiting progression of the disease, disorder or condition, causing regression of the disease, disorder or condition and/or curing the disease, disorder or condition.

Various techniques to modify or engineer an ABP of the invention to increase ADCC are known (Satoh et al, 2006; Expert Opin Biol Ther 6:1161; WO2009/135181), and hence such embodiments include those wherein an ABP of the invention may be afucosylated (GlycArt Biotechnology) e.g., in which antibodies are produced in CHO cells in which the endogenous FUT8 gene has been knocked out; or the ABP may be a “Sugar-Engineered Antibody” (Seattle Genetics), e.g. in which fucose analogues are added to antibody-expressing CHO cells, resulting in a significant reduction in fucosylation. Other afucosylation approaches that may be applied to an ABP of the invention are described elsewhere herein.

Other techniques to modify or engineer an ABP of the invention to increase ADCC include mutations in a Fc portion of the ABP, (such as described in more detail elsewhere herein), in particular where one or more of residues 234, 235, 236 and/or 237, and/or residues 330, 331 of human Fc are so mutated; wherein such numbering of the residues in the Fc region is that of the EU index as in Kabat (Kabat et ah, Sequences of Proteins of Immunological Interest (National Institute of Health, Bethesda, Md. 1987).

Accordingly, in certain embodiments, the ABP of the invention is modified or engineered to increase antibody-dependent cell-mediated cytotoxicity (ADCC), preferably wherein said ABP is afucosylated and/or an Fc of said ABP is mutated. In alternative embodiments, the ABP of the invention is modified or engineered to reduce ADCC (eg where an Fc is mutated using one or more of the following residue changes: L234A, L235E, G237A, A330S and/or P331S).

In other certain embodiments, the ABP of the invention is modified to prolong serum half-life, especially in human serum. For example, an ABP of the invention may be PEGylated and/or PASylated, or has an Fc region with a T250Q/M428L, H433K/N434F/Y436 or M252Y/S254T/T256E/H433K/N434F modification.

In certain embodiments, the ABP of the invention binds to (a) one or more epitopes displayed by an extracellular domain of IGSF11, or the variant of IGSF11; or which binds to (b) two or more epitopes displayed by an extracellular domain of IGSF11, or the variant of IGSF11. Preferably, one or more of said epitopes is displayed between amino acid residues 23 and 241 (ECD of human IGSF11 protein) of SEQ ID NO: 371, such as between amino acid residues 23 and 136 (Ig-like V-type domain of human IGSF11 protein) of SEQ ID NO: 371.

An ABP of the present invention may be mono-specific (i.e, it possesses antigen binding domain(s) that bind to only one antigen) or may be multi-specific (i.e, it possesses two or more different antigen binding domain(s) that bind to different antigens). For example, a “bi-specific” “dual-specific” or “bifunctional” ABP or antibody is a hybrid ABP or antibody, respectively, having two different antigen binding sites. Bi-specific antigen binding proteins and antibodies are a species of multi-specific antigen binding protein antibody and can be produced by a variety of methods including, but not limited to, fusion of hybridomas or linking of Fab′ fragments (see, e.g., Songsivilai and Lachmann, 1990; Kostelny et al., 1992). The two binding sites of a bi-specific antigen binding protein or antibody will bind to two different epitopes, which can reside on the same or different protein targets.

In certain of such embodiments, the ABP may be a bi-specific, tri-specific, or tetra-specific antibody, in particular a bi-specific antibody is selected from: a bispecific T-cell engager (BiTE) antibody, a dual-affinity retargeting molecule (DART), a CrossMAb antibody, a DutaMab™ antibody, a DuoBody antibody; a Triomab, a TandAb, a bispecific NanoBody, Tandem scFv, a diabody, a single chain diabody, a HSA body, a (scFv)2 HSA Antibody, an scFv-IgG antibody, a Dock and Lock bispecific antibody, a DVD-IgG antibody, a TBTI DVD-IgG, an IgG-fynomer, a Tetravalent bispecific tandem IgG antibody, a dual-targeting domain antibody, a chemically linked bispecific (Fab′)2 molecule, a crosslinked mAb, a Dual-action Fab IgG (DAF-IgG), an orthoFab-IgG, a bispecific CovX-Body, a bispecific hexavalent trimerbody, and an ART-Ig.

Accordingly, in certain embodiments, the ABP of the invention is a multi-specific antibody comprising at least two antigen binding domains, wherein each antigen binding domain specifically binds to a different antigen epitope.

In certain of such embodiments of such an ABP, at least two of the different antigen epitopes are epitopes displayed by the ECD of IGSF11 (VSIG3) protein of by the IgC2 (or IgV) domain of IGSF11, or wherein at least one of the different antigen epitopes is an epitope displayed by the ECD of IGSF11 (VSIG3) protein of by the IgC2 (or IgV) domain of IGSF11, and at least one of the different antigen epitopes is an epitope displayed by a protein other than IGSF11 (VSIG3), and preferably other than an epitope displayed by a protein other than VSIR (VISTA), or other than another interacting protein to IGSF11 protein.

Accordingly, in some embodiments, the ABP of the invention binds (e.g. via one or more first antigen binding domain(s)) to an extracellular domain(s) of the IGSF11 (VSIG3), paralogue, orthologue or other variant, of to the IgC2 (or IgV) domain thereof, when expressed on the surface of a mammalian cell, and in addition comprises one or more additional antigen binding domain(s) that bind(s) to antigen(s) other than said IGSF11 (VSIG3) or variant or domain. Such other antigen may, in certain embodiments of the inventive ABP, be another immunoglobulin superfamily gene (preferably not VSIR); and/or such other antigen may be an antigen present on a mammalian T-cell. Antigens present on a mammalian T-cell, that may be bound by such an additional antigen binding domain, include CD3, CD40, OX-40, ICOS and 4-1BB. Such other antigen may, in certain embodiments of the inventive ABP, also be albumin, e.g., human albumin. It may also be another component of blood or blood serum the binding of which by the ABP will confer an extended serum half-life upon the ABP, e.g., a half-life similar to that when bound to albumin.

In other embodiments, an ABP of the invention can comprise two or more antigen binding regions, preferably comprising two, three or four antigen binding regions.

In yet other embodiments, an ABP of the invention can comprise a chimeric antigen receptor (CAR), and preferably comprises an extracellular antigen binding region, a membrane anchor such as a transmembrane domain, and an intracellular region, for example, an intracellular signalling region.

In preferred embodiments, an ABP of the invention can comprise at least one antibody constant domain, in particular wherein at least one antibody constant domain is a CH1, CH2, or CH3 domain, or a combination thereof.

In further of such embodiments, an ABP of the invention having antibody constant domain comprises a mutated Fc region, for example for increasing interaction of the Fc region with a Fc receptor (Fc receptor on an immune effector cell (eg Saxena & Wu, 2016; Front Immunol 7:580). Examples and embodiments thereof are described elsewhere herein.

In other embodiments, an ABP of the invention may comprises an effector group and/or a labelling group.

The term “effector group” means any group, in particular one coupled to another molecule such as an antigen binding protein, that acts as a cytotoxic agent. Examples for suitable effector groups are radioisotopes or radionuclides. Other suitable effector groups include toxins, therapeutic groups, or chemotherapeutic groups. Examples of suitable effector groups include calicheamicins, auristatins, geldanamycins, alpha-amanitine, pyrrolobenzodiazepines and maytansines.

The term “label” or “labelling group” refers to any detectable label. In general, labels fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, β-galactosidase, luciferase, alkaline phosphatase); e) biotinylated groups; and f) predetermined polypeptide epitopes recognized by a secondary reporter (e.g., leucine zipper pair sequences, binding sites for secondary antibodies, metal binding domains, epitope tags, etc.).

In some embodiments, an effector group or a labelling group is coupled to another molecule (such as the ABP) via spacer arms of various lengths to reduce potential steric hindrance.

In another aspect, the invention relates to an antigen binding domain (ABD) of an ABP of the invention, such as of any ABP as described above or elsewhere herein. In certain embodiments, an ABD of the invention is capable, when comprised in an applicable scaffold, of binding to the ECD of IGSF11 (or variant thereof). An ABD of the invention may, in certain embodiments, be isolated and/or substantial pure.

Nucleic Acids, Nucleic Acid Constructs and (Host) Cells

In a third aspect, the invention relates to a nucleic acid encoding for an ABP (or ABD) of the invention (such as one described above) or of components thereof. For example, the component encoded by a nucleic acid of the invention may be all or part of one chain of an antibody of the invention; or the component may be a scFV of said ABP. The component encoded by such a nucleic acid may be all or part of one or other of the chains of an antibody of the invention; for example, the component encoded by such a nucleic acid may be an ABD of the invention. The nucleic acids of the invention may also encode a fragment, derivative, mutant, or variant of an ABP of the invention, and/or represent components that are polynucleotides suitable and/or sufficient for use as hybridisation probes, polymerase chain reaction (PCR) primers or sequencing primers for identifying, analyzing, mutating or amplifying a polynucleotide encoding a polypeptide, anti-sense or inhibitory nucleic acids (such as RNAi/siRNA/shRNA or gRNA molecules) for inhibiting expression of a polynucleotide, and complementary sequences of the foregoing.

In particular embodiments of the invention, a nucleic acid of the invention comprises a nucleic acid having a sequence encoding a heavy or light chain CDR, a combination of heavy and/or light chain CDR1, CDR2 and CDR3 or a heavy or light chain variable domain, in each case as displayed in Table 13.1A, or a functional fragment thereof. In other embodiments, a nucleic acid of the invention comprises a nucleic acid sequence at least 60%, 65%, 70%, 75%, 80%, 85%, 90%; or 95% (preferably at least 75%) sequence identity to (or having no more than fifty, forty, thirty, twenty, fifteen, ten or five, preferably no more than three, two or one, base substitution(s), insertion(s) or deletion(s) (in particular, substitution(s)), preferably at the third base of a codon of) a nucleic acid sequence selected from the list consisting of SEQ IDS Nos. 399, 400, 409, 410, 419, 420, 429, 430, 439, 440, 449, 450, 459, 460, 469, 470, 479, 480, 489, 490, 499, 500, 509, 510, 519, 520, 529, 530, 539, 540, 549, 550, 559, 560, 569, 570, 579, 580, 589, 590, 599, 600, 609, 610, 619, 620, 629, 630, 639, 640, 649, 650, 659, 660, 669, 670, 679 and 680 (in particular, a nucleic acid sequence of the corresponding heavy and/or light chain variable domain shown in Table 13.1A for an antibody selected from any one of the antibodies of the group consisting of: C-002, C-003, C-004, C-005, C-006, C-010, C-011, C-013, C-014, C-015, C-018, C-021, C-022 and C-023, preferably C-003, C-004 or C-005 (eg, C-005), and/or selected from any one of the antibodies of the group consisting of: C-001, C-007, C-008, C-009, C-016, C-017, C-024, C-025 and C-026, preferably C-001 or C-007; preferably wherein such nucleic acid encodes a heavy or light chain variable domain of an ABP of the invention, such as encodes the corresponding heavy or light chain variable domain having the amino acid sequence set forth in Table 13.1A, and optionally having no more than fifteen, fourteen, thirteen, twelve or eleven (eg, for variable light chain), such as no more than ten, nine, eight, seven, six, five, four, preferably no more than three, two or one, amino acid substitution(s), insertion(s) or deletion(s) (in particular, substitution(s)) compared to these sequences.

The nucleic acid according to the invention may be a DNA or RNA of genomic, mRNA, cDNA, or synthetic origin or some combination thereof, optionally linked to a polynucleotide to which it is not linked in nature. In some embodiments, such nucleic acid may comprise one or more (such as 2, 3, 4, 5, 6, 7, 8, 9, 10 or more than 20, in particular between 1 and about 5, or preferably all instances of a particular nucleotide in the sequence) unnatural (e.g. synthetic) nucleotides; and/or such nucleic acid may comprise (e.g. is conjugated to) another chemical moiety, such as a labelling group or an effector group; for example, a labelling group or an effector group as described elsewhere herein.

In one embodiment, the nucleic acid of the invention may be isolated or substantially pure. In another embodiment, the nucleic acid of the invention may be recombinant, synthetic and/or modified, or in any other way non-natural. For example, a nucleic acid of the invention may contain at least one nucleic acid substitution (or deletion) modification (such as 2, 3, 4, 5, 6, 7, 8, 9, 10 or more than 10 such modifications, in particular between 1 and about 5 such modifications, preferably 2 or 3 such modifications) relative to a product of nature, such as a human nucleic acid.

The nucleic acids can be any suitable length, such as about 10, 15, 20, 25, 30, 35, 40, 45, 50, 75, 100, 125, 150, 175, 200, 250, 300, 350, 400, 450, 500, 750, 1,000, 1,500, 3,000, 5,000 or more nucleotides in length. For example: siRNA nucleic acids may, preferably, be between about 15 to about 25 base pairs in length (preferably between about 19 and about 21 base pairs in length); shRNA nucleic acids may, preferably, comprise a 20-30 base pair stem, a loop of at least 4 nucleotides, and a dinucleotide overhang at the 3′ end; microRNA may, preferably, be about 22 base pairs in length; an mRNA or DNA sequence encoding an ABP or a component thereof (such as a heavy or light chain or an IgG antibody) of the invention may, preferably, be between about 500 and 1,500 nucleotides. More preferably, a nucleic acid encoding a mammalian light chain of an antibody may be between about 630 and about 650 nucleotides, and one encoding a mammalian heavy chain of an antibody may be between about 1,300 and about 1,650 nucleotides. A nucleic acid can comprise one or more additional sequences, for example, regulatory sequences, and/or be part of a larger nucleic acid. The nucleic acids can be single-stranded or double-stranded and can comprise RNA and/or DNA nucleotides, and artificial variants thereof (e.g., peptide nucleic acids).

Nucleic acids encoding antibody polypeptides (e.g., heavy or light chain, variable domain only, or full length) may be isolated from B-cells of mice, rats, llamas, alpacas, goat, chicken or rabbits that have been immunized with an IGSF11 (VSIG3) antigen or fragment thereof, such as one or more EC domains (or a polynucleotide encoding and capable of expressing an IGSF11 antigen or fragment thereof), and in particular with an IgC2 domain of IGSF11 (or and IgV domain of IGSF11), or a polynucleotide encoding and capable of expressing such domain or fragment thereof. The nucleic acid may be isolated by conventional procedures such as PCR.

Changes can be introduced by mutation into the sequence of a nucleic acid of the invention. Such changes, depending on their nature and location in a codon, can lead to changes in the amino acid sequence of a polypeptide (e.g., an antigen binding protein) that it encodes. Mutations can be introduced using any technique known in the art.

In one embodiment, one or more particular amino acid residues may be changed using, for example, a site-directed mutagenesis protocol. In another embodiment, one or more randomly selected residues may be changed using, for example, a random mutagenesis protocol. However, it is made, a mutant polypeptide can be expressed and screened for a desired property. Mutations can be introduced into a nucleic acid without significantly altering the biological activity of a polypeptide that it encodes. For example, one can make nucleotide substitutions leading to amino acid substitutions at non-essential amino acid residues.

Other changes that may be made (e.g. by mutation) to the sequence of a nucleic acid of the invention may not alter the amino acid sequence of the encoded polypeptide, but may lead to changes to its stability and/or effectiveness of expression of the encoded polypeptide. For example, by codon optimisation, the expression of a given polypeptide sequence may be improved by utilising the more common codons for a given amino acid that are found for the species in which the nucleotide is to be expressed. Methods of codon optimisation, and alternative methods (such as optimisation of CpG and G/C content), are described in, for example, Hass et al, 1996 (Current Biology 6:315); WO1996/09378; WO2006/015789 and WO 2002/098443).

In one related aspect, the invention relates to a nucleic acid construct (NAC) comprising at least one nucleic acid of the invention (such as described above). Such an NAC can comprise one or more additional features permitting the expression of the encoded ABP or component of said ABP (eg the ABD) in a cell (such as in a host cell). Examples of NACs of the invention include, but are not limited to, plasmid vectors, viral vectors, mRNA, non-episomal mammalian vectors and expression vectors, for example, recombinant expression vectors. The nucleic acid constructs of the invention can comprise a nucleic acid of the invention in a form suitable for expression of the nucleic acid in a cell, such as a host cell, (see below). The nucleic acid constructs of the invention will be, typically, recombinant nucleic acids, and/or may be isolated and/or substantially pure. Recombinant nucleic acids will, typically, be non-natural; particularly if they comprise portions that are derived from different species and/or synthetic, in-vitro or mutagenic methods.

In some embodiments, an NAC of the invention comprises one or more constructs either of which includes a nucleic acid encoding either a heavy or a light antibody chain. In some embodiments, the NAC of the invention comprises two constructs, one of which includes a nucleic acid encoding the heavy antibody chain, the other of which includes a nucleic acid encoding the light antibody chain, such that expression from both constructs can generate a complete antibody molecule. In some embodiments, the NAC of the invention comprises a construct which includes nucleic acids encoding both heavy and light antibody chains, such that a complete antibody molecule can be expressed from one construct. In other embodiments, an NAC of the invention can comprise a single construct that encodes a single chain which is sufficient to form an ABP of the invention; for example, if the encoded ABP is a scFv or a single-domain antibody (such as a camelid antibody).

In some embodiments, the NAC of the invention includes sequences encoding all or part of a constant region, enabling an entire, or a part of, a heavy and/or light chain to be expressed.

An NAC according to the invention may comprise (or consist of) a mRNA molecule which includes an open reading frame encoding an ABP of the invention, and for example together with upstream and downstream elements (such as 5′ and/or 3′ UTRs and/or poly-A stretch) that enables expression of the ABP, and preferably enhancing stability of the mRNA and/or expression of the ABP. The use of mRNA as NACs to introduce into and express polynucleotides in cells is described, for example, in Zangi et al in Nat. Biotechnol. vol. 31, 898-907 (2013), Sahin et al (2014) Nature Reviews Drug Discovery 13:759 and by Thess et al in Mol. Ther. vol. 23 no. 9, 1456-1464 (2015). Particular UTRs that may be comprised in an mRNA NAC of the invention include: 5′UTR of a TOP gene (WO2013/143699), and/or a histone stem-loop (WO 2013/120629). An mRNA NAC of the invention may further comprise one or more chemical modifications (EP 1 685 844); including a 5′-cap, such as m7G(5′)ppp, (5′(A,G(5′)ppp(5′)A or G(5′)ppp(5′)G and/or at least one nucleotide that is an analogue of naturally occurring nucleotides, such as phosphorothioates, phosphoroamidates, peptide nucleotides, methylphosphonates, 7-deaza-guanosine, 5-methylcytosine or inosine.

NACs, such as DNA-, retroviral- and mRNA-based NACs of the invention may be used in genetic therapeutic methods in order to treat or prevent diseases of the immune system (see Methods of Treatment below), whereby an NAC that comprises an expressible sequence encoding an ABP of the invention is administered to the cell or organism (e.g. by transfection). In particular, the use of mRNA therapeutics for the expression of antibodies is known from WO2008/083949.

In another related aspect, the invention relates to a cell (such as a host cell and/or a recombinant host cell) comprising one or more nucleic acid or NAC of the invention. Preferably, such cell is capable of expressing the ABP (or component thereof) encoded by said NAC(s). For example, if an ABP of the invention comprises two separate polypeptide chains (e.g. a heavy and light chain of an IgG), then the cell of the invention may comprise a first NAC that encodes (and can express) the heavy chain of such ABP as well as a second NAC that encodes (and can express) the light chain of such ABP; alternatively, the cell may comprise a single NAC that encodes both chains of such ABP. In these ways, such a cell of the invention would be capable of expressing a functional (e.g. binding and/or inhibitory) ABP of the invention. A (host) cell of invention may be one of the mammalian, prokaryotic or eukaryotic host cells as described elsewhere herein, in particularly where the cell is a Chinese hamster ovary (CHO) cell.

In certain embodiments of such aspect, the (host) cell is a human cell; in particular it may be a human cell that has been sampled from a specific individual (eg an autologous human cell, such as an autologous human T cell engineered to express an ABP of the invention as a chimeric antigen receptor). In such embodiments, such human cell can be propagated and/or manipulated in-vitro so as to introduce a NAC of the present invention. The utility of a manipulated human cell from a specific individual can be to produce an ABP of the invention, including to reintroduce a population of such manipulated human cells into a human subject, such as for use in therapy. In certain of such uses, the manipulated human cell may be introduced into the same human individual from which it was first sampled; for example, as an autologous human cell.

The human cell that is subject to such manipulation can be of any germ cell or somatic cell type in the body. For example, the donor cell can be a germ cell or a somatic cell selected from the group consisting of fibroblasts, B cells, T cells, dendritic cells, keratinocytes, adipose cells, epithelial cells, epidermal cells, chondrocytes, cumulus cells, neural cells, glial cells, astrocytes, cardiac cells, oesophageal cells, muscle cells, melanocytes, hematopoietic cells, macrophages, monocytes, and mononuclear cells. The donor cell can be obtained from any organ or tissue in the body; for example, it can be a cell from an organ selected from the group consisting of liver, stomach, intestines, lung, pancreas, cornea, skin, gallbladder, ovary, testes, kidneys, heart, bladder, and urethra.

Pharmaceutical Compositions

To be used in therapy, the ABPs, nucleic acids or NACs (or the cells, such as host cells) of the invention may be formulated into a pharmaceutical composition appropriate to facilitate administration to animals or humans. The term “pharmaceutical composition” means a mixture of substances including a therapeutically active substance (such as an ABP of the invention) for pharmaceutical use.

Accordingly, in a fourth aspect, the invention relates to a pharmaceutical composition comprising a compound that specifically binds to and/or is a modulator of the expression, function, activity and/or stability of immunoglobulin superfamily member 11 (IGSF11, or VSIG3), or of a C2-type immunoglobulin-like (IgC2) domain of IGSF11 (or, in another aspect, specifically binds to and/or is an modulator of the expression, function, activity and/or stability of a V-type immunoglobulin-like (IgV) domain of IGSF11), or of a variant thereof and a pharmaceutically acceptable carrier, stabiliser and/or excipient. In certain embodiments, the compound that specifically binds to and/or modulator is not an ABP that is the subject of one or more of the provisos (A), (B), (C), (D), (E) and/or (F) as set out elsewhere herein. For example, the IGSF11 compound and/or modulator is an ABP of the invention, and/or at least one NAC of the invention, and/or a (host) cell of the invention. Accordingly, in a related aspect, herein provided is a pharmaceutical composition comprising an ABP of the invention, and/or at least one NAC of the invention, and/or a (host) cell of the invention, and a pharmaceutically acceptable excipient or carrier.

In a preferred embodiment, the pharmaceutical composition comprises an ABP of the invention, for example in such embodiment, the IGSF11 compound and/or modulator is an ABP of the invention (eg an IGSF11-inhibitory ABP of the invention, such as an inhibitor of an IgC2 domain of IGSF11, an inhibitor of an IgV domain of IGSF11).

By way of example, the pharmaceutical composition of the invention may comprise between 0.1% and 100% (w/w) active ingredient (for example, an ABP specially binding to IGSF, an IGSF11 modulator or an ABP specially binding to, and/or a modulator of, an IgC2 or IgV domain of IGSF11), such as about 0.5%, 1%, 2%, 3%, 4%, 5%, 6%, 8% 10%, 15%, 20%, 25%, 30%, 40%, 50%, 60%, 70%, 80%, 90%, 95%, 96%, 97%, 98% or 99%, preferably between about 1% and about 20%, between about 10% and 50% or between about 40% and 90%.

As used herein the language “pharmaceutically acceptable” excipient, stabiliser or carrier is intended to include any and all solvents, solubilisers, fillers, stabilisers, binders, absorbents, bases, buffering agents, lubricants, controlled release vehicles, diluents, emulsifying agents, humectants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The pharmaceutical composition of (or for use with) the invention is, typically, formulated to be compatible with its intended route of administration. Examples of routes of administration include oral, parenteral, e.g., intrathecal, intra-arterial, intravenous, intradermal, subcutaneous, oral, transdermal (topical) and transmucosal administration.

Solutions or suspensions used for parenteral, intradermal, or subcutaneous application, as well as comprising a compound of (or for use with) the invention (eg an IGSF11/domain binder and/or modulator), can include the following components: a sterile diluent such as water for injection, saline solution, fixed oils, polyethylene glycols, glycerine; propylene glycol or other synthetic solvents; anti-bacterial agents such as benzyl alcohol or methyl parabens; antioxidants such as ascorbic acid or sodium bisulphate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Kolliphor® EL (formerly Cremophor EL™; BASF, Parsippany, N.J.) or phosphate buffered saline (PBS). In all cases, the injectable composition should, typically, be sterile and be fluid to the extent that easy syringability exists. It should, typically, be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminium monostearate and gelatin.

Sterile injectable solutions can be prepared by incorporating the compound of (or for use with) the invention (e.g., an IGSF11/domain binder and/or modulator) in the required amount in an appropriate solvent with one or a combination of ingredients described herein, as required, followed by filtered sterilisation. Generally, dispersions are prepared by incorporating the active compound into a sterile vehicle which contains a basic dispersion medium and the required other ingredients from those described herein. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions, as well as comprising a compound of (or for use with) the invention (eg an IGSF11/domain inhibitor), generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavouring agent such as peppermint, methyl salicylate, or orange flavouring.

Furthermore, the compounds of (or for use with) the invention (eg an IGSF11/domain binder and/or modulator) can be administered rectally. A rectal composition can be any rectally acceptable dosage form including, but not limited to, cream, gel, emulsion, enema, suspension, suppository, and tablet. One preferred dosage form is a suppository having a shape and size designed for introduction into the rectal orifice of the human body. A suppository usually softens, melts, or dissolves at body temperature. Suppository excipients include, but are not limited to, theobroma oil (cocoa butter), glycerinated gelatin, hydrogenated vegetable oils, mixtures of polyethylene glycols of various molecular weights, and fatty acid esters of polyethylene glycol.

For administration by inhalation, the compounds of (or for use with) the invention (eg an IGSF11 binder and/or modulator) are typically delivered in the form of an aerosol spray from pressured container or dispenser which contains a suitable propellant, e.g., a gas such as carbon dioxide, or a nebuliser.

Cells, such as immune cells (eg CART cells, such as a host cell of the invention, eg an autologous human T cell engineered to express an ABP of the invention as a chimeric antigen receptor) for use with the invention can be included in pharmaceutical formulations suitable for administration into the bloodstream or for administration directly into tissues or organs. A suitable format is determined by the skilled person (such as a medical practitioner) for each patient, tissue, and organ, according to standard procedures. Suitable pharmaceutically acceptable carriers and their formulation are known in the art (see, e.g. Remington's Pharmaceutical Sciences 16th edition, Osol, A. Ed., 1980). Such cells, when formed in a pharmaceutical composition, are preferably formulated in solution at a pH from about 6.5 to about 8.5. Excipients to bring the solution to isotonicity can also be added, for example, 4.5% mannitol or 0.9% sodium chloride, pH buffered with art-known buffer solutions, such as sodium phosphate. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes. In one embodiment, a media formulation is tailored to preserve the cells while maintaining cell health and identity. For example, a premixture including an aqueous solution of anticoagulant (ACD-A), an equal amount of dextrose (50%), and phosphate buffered saline (PBS), or the like is pre-mixed and aliquoted in a volume to typically match or approximate the cellular matrix or environment from which the cell was extracted from the tissue or organ.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions can be formulated into ointments, salves, gels, or creams as generally known in the art.

In certain embodiments, the pharmaceutical composition is formulated for sustained or controlled release of a compound of (or for use with) the invention (eg an IGSF11/domain binder and/or modulator). Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially (including liposomes targeted to infected cells with monoclonal antibodies to viral antigens) can also be used as pharmaceutically acceptable carriers. These can be prepared according to methods known to those skilled in the art.

It is especially advantageous to formulate oral, rectal or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

In some embodiments, the pharmaceutical composition comprising an IGSF11/domain binder and/or modulator is in unit dose form of between 10 and 1000 mg IGSF11 binder and/or modulator. In some embodiments, the pharmaceutical composition comprising an IGSF11/domain binder and/or modulator is in unit dose form of between 10 and 200 mg binder and/or modulator. In some embodiments, the pharmaceutical composition comprising an ABP is in unit dose form of between 200 and 400 mg binder and/or modulator. In some embodiments, the pharmaceutical composition comprising an IGSF11/domain binder and/or modulator is in unit dose form of between 400 and 600 mg binder and/or modulator. In some embodiments, the pharmaceutical composition comprising an IGSF11/domain binder and/or modulator is in unit dose form of between 600 and 800 mg binder and/or modulator. In some embodiments, the pharmaceutical composition comprising an IGSF11/domain binder and/or modulator is in unit dose form of between 800 and 100 mg binder and/or modulator.

Exemplary unit dosage forms for pharmaceutical compositions comprising IGSF11/domain modulators are tablets, capsules (eg as powder, granules, microtablets or micropellets), suspensions or as single-use pre-loaded syringes. In certain embodiments, kits are provided for producing a single-dose administration unit. The kit can contain both a first container having a dried active ingredient and a second container having an aqueous formulation. Alternatively, the kit can contain single and multi-chambered pre-loaded syringes.

Toxicity and therapeutic efficacy (eg effectiveness) of such active ingredients can be determined by standard pharmaceutical procedures in cell cultures or experimental animals, eg, for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Active agents which exhibit large therapeutic indices are preferred. While compounds that exhibit toxic side effects may be used, care should be taken to design a delivery system that targets such compounds to the site of affected tissue in order to minimise potential damage to uninfected cells and, thereby, reduce side effects.

The data obtained from the cell culture assays and animal studies can be used in formulating a range of dosage of the active ingredients (eg an IGSF11/domain binder and/or modulator), such as for use in humans. The dosage of such active ingredients lies preferably within a range of circulating concentrations that include the ED50 with little or no toxicity. The dosage may vary within this range depending upon the dosage form employed and the route of administration utilised. For any active ingredients used in the therapeutic approaches of the invention, the (therapeutically) effective dose can be estimated initially from cell culture assays. A dose may be formulated in animal models to achieve a circulating plasma concentration range that includes the IC50 (ie, the concentration of the active ingredients which achieves a half-maximal inhibition of symptoms) as determined in cell culture. Such information can be used to more accurately determine useful (eg effective) amounts or doses, such as for administration to humans. The pharmaceutical compositions can be included in a container, pack, or dispenser together with instructions for administration.

In the context of the invention, an effective amount of the IGSF11/domain binder and/or modulator or the pharmaceutical composition can be one that will elicit the biological, physiological, pharmacological, therapeutic or medical response of a cell, tissue, system, body, animal, individual, patient or human that is being sought by the researcher, scientist, pharmacologist, pharmacist, veterinarian, medical doctor, or other clinician, eg, lessening of the effects/symptoms of a disorder, disease or condition, such as a proliferative disorder, for example, a cancer or tumour, or killing or inhibiting growth of a cell involved with a proliferative disorder, such as a tumour cell. The effective amount can be determined by standard procedures, including those described below.

In accordance with all aspects and embodiments of the medical uses and methods of treatment provided herein, the effective amount administered at least once to a subject in need of treatment with an IGSF11/domain binder and/or modulator is, typically, between about 0.01 mg/kg and about 100 mg/kg per administration, such as between about 1 mg/kg and about 10 mg/kg per administration. In some embodiments, the effective amount administered at least once to said subject of a IGSF11/domain binder and/or modulator is between about 0.01 mg/kg and about 0.1 mg/kg per administration, between about 0.1 mg/kg and about 1 mg/kg per administration, between about 1 mg/kg and about 5 mg/kg per administration, between about 5 mg/kg and about 10 mg/kg per administration, between about 10 mg/kg and about 50 mg/kg per administration, or between about 50 mg/kg and about 100 mg/kg per administration.

For the prevention or treatment of disease, the appropriate dosage of a IGSF11/domain binder and/or modulator (or a pharmaceutical composition comprised thereof) will depend on the type of disease to be treated, the severity and course of the disease, whether the IGSF11/domain binder and/or modulator and/or pharmaceutical composition is administered for preventive or therapeutic purposes, previous therapy, the patient's clinical history, age, size/weight and response to the IGSF11/domain binder and/or modulator and/or pharmaceutical composition, and the discretion of the attending physician. The IGSF11/domain binder and/or modulator and/or pharmaceutical composition is suitably administered to the patient at one time or over a series of treatments. If such IGSF11/domain inhibitor and/or pharmaceutical composition is administered over a series of treatments, the total number of administrations for a given course of treatment may consist of a total of about 2, 3, 4, 5, 6, 7, 8, 9, 10 or more than about 10 treatments. For example, a treatment may be given once every day (or 2, 3 or 4 times a day) for a week, a month or even several months. In certain embodiments, the course of treatment may continue indefinitely.

The amount of the IGSF11/domain binder and/or modulator and/or pharmaceutical composition administered will depend on variables such as the type and extent of disease or indication to be treated, the overall health, age, size/weight of the patient, the in vivo potency of the IGSF11/domain binder and/or modulator and/or pharmaceutical composition, and the route of administration. The initial dosage can be increased beyond the upper level in order to rapidly achieve the desired blood-level or tissue level. Alternatively, the initial dosage can be smaller than the optimum, and the daily dosage may be progressively increased during the course of treatment. Human dosage can be optimised, e.g., in a conventional Phase I dose escalation study designed to run from relatively low initial doses, for example from about 0.01 mg/kg to about 20 mg/kg of active ingredient. Dosing frequency can vary, depending on factors such as route of administration, dosage amount and the disease being treated. Exemplary dosing frequencies are once per day, once per week and once every two weeks. Formulation of an IGSF11/domain binder and/or modulator of (or for use with) the present is within the ordinary skill in the art. In some embodiments of the invention such IGSF11/domain binder and/or modulator is lyophilised and reconstituted in buffered saline at the time of administration. The IGSF11/domain binder and/or modulator and/or pharmaceutical composition of may further result in a reduced relapsing of the disease to be treated or reduce the incidence of drug resistance or increase the time until drug resistance is developing; and in the case of cancer may result in an increase in the period of progression-free survival and/or overall survival.

Modulators of the Expression, Function, Activity and/or Stability of IGSF11 (VSIG3) and IGSF11/Domain Binders, Including for Use in Pharmaceutical Compositions and Therapy

In one embodiment of these aspects, the compound that is an IGSF/domain binder and/or is a modulator of the expression, function, activity and/or stability of immunoglobulin superfamily member 11 (IGSF11, or VSIG3) or of a C2-type immunoglobulin-like (IgC2) domain of IGSF11 (or, in another aspect, is a modulator of the expression, function, activity and/or stability of a V-type immunoglobulin-like (IgV) domain of IGSF11 (VSIG3)), or of a variant thereof (such as described above), is a compound that is an an inhibitor or antagonist of expression, function, activity and/or stability of IGSF11 or of such domain, or of the variant thereof, in particular a compound that inhibits the binding of an interacting protein (such as VSIR protein, or a variant thereof) to IGSF11 protein (or a variant thereof), in particular inhibits the binding of human VSIR protein (or a variant thereof) to human IGSF11 protein or to the IgC2 (or IgV) domain of human IGSF11 protein (or a variant thereof), such as inhibits the binding between the ECDs of VSIR protein the IgC2 (or IgV) domain of human IGSF11 protein, as for example, as described above.

In an alternative embodiment of these aspects, the compound that is an IGSF/domain binder and/or is a modulator of the expression, function, activity and/or stability of immunoglobulin superfamily member 11 (IGSF11, or VSIG3) or of a C2-type immunoglobulin-like (IgC2) domain of IGSF11 (or, in another aspect, is a modulator of the expression, function, activity and/or stability of a V-type immunoglobulin-like (IgV) domain of IGSF11 (VSIG3)), or of a variant thereof (such as described above), is a compound that is an an activator or agonist of expression, function, activity and/or stability of IGSF11 or of such domain, or of the variant thereof, in particular a compound that triggers the receptor signaling pathway of the IGSF11 or variant of.

In either of such embodiments, the compound can be one selected from a polypeptide, peptide, glycoprotein, a peptidomimetic, an antigen binding protein (ABP) (for example, an antibody, antibody-like molecule or other antigen binding derivative, or an or antigen binding fragment thereof), a nucleic acid such as a DNA or RNA, for example an antisense or inhibitory DNA or RNA, a ribozyme, an RNA or DNA aptamer, RNAi, siRNA, shRNA and the like, including variants or derivatives thereof such as a peptide nucleic acid (PNA), a genetic construct for targeted gene editing, such as a CRISPR/Cas9 construct and/or a guide nucleic acid (gRNA or gDNA) and/or tracrRNA and a hetero bi-functional compound such as a PROTAC or HyT molecule.

In a preferred embodiment, the compound is an antigen binding protein (ABP) of the invention, such as one of the first or second aspects. For example, the compound can be such an ABP that is not an ABP that is the subject of one or more of the provisos (A), (B), (C), (D), (E) and/or (F) as set out elsewhere herein,

In particular embodiments, the compound enhances killing and/or lysis of cells expressing IGSF11, or a variant of IGSF11, by cytotoxic T-cells and/or TILs.

In other particular embodiments, a compound modulator of the invention that is an inhibitor or antagonist of IGSF11 expression, function, activity and/or stability can mediate any one, or a combination or at least one, functional characteristic of the inhibiting or antagonistic modulators described herein, in particular in the section above “Modulators of IGSF11 expression, function, activity and/or stability”.

A compound modulator of the invention that is an activator or agonist of IGSF11 expression, function, activity and/or stability, or that is an activator or agonist of expression, function, activity and/or stability of an IgC2 (or IgV) domain of IGSF11, can mediate any one, or a combination or at least one, functional characteristic of the activating or agonistic modulators described herein, in particular in the section above “Modulators of IGSF11 expression, function, activity and/or stability”.

The compound can, in one embodiment, comprise an ECD of an IGSF11 protein (eg, can comprise an IgC2 (or IgV) domain of IGSF11 protein) or of a VSIR protein, in particular of a human IGSF11 protein or of a human VSIR protein. Such ECDs and IgC2 (or IgV) domains are described elsewhere herein.

The compound can, in a preferred embodiment, comprise an ABP (for example, an antibody, antibody-like molecule or other antigen binding derivative, or an antigen binding fragment thereof), that binds said IGSF11 or said domain of IGSF11, or the variant thereof, in particular an ABP of the invention described elsewhere herein.

Alternatively, in another preferred embodiment, the compound can be a nucleic acid (for example an anti-sense nucleotide molecule such as a siRNA or shRNA molecule) that binds to a nucleic acid that encodes or regulates the expression of a gene that controls the expression, function, activity and/or stability of IGSF11 or of such domain of IGSF11, or of a variant thereof. For example, in particular of such embodiments, the nucleic acid (for example an anti-sense nucleotide molecule such as a siRNA or shRNA molecule) that binds to a nucleic acid that encodes IGSF11 or encodes such domain of IGSF11, or of a variant thereof, or that binds a nucleic acid that regulates the expression of encodes IGSF11 or of such domain of IGSF11, or of a variant thereof IGSF11, or that binds to a nucleic acid that encodes for a gene that regulates the expression of encodes IGSF11 of such domain, or such variant thereof.

Nucleic Acid Modulating Compounds

As described above, in one particular set of embodiments, the compound modulator is a nucleic acid.

The terms “nucleic acid” “polynucleotide” and “oligonucleotide” are used, in this context, interchangeably throughout and include DNA molecules (e.g., cDNA or genomic DNA), RNA molecules (e.g., mRNA), analogues of the DNA or RNA generated using nucleotide analogues (e.g., peptide nucleic acids and non-naturally occurring nucleotide analogues), and hybrids thereof. The nucleic acid molecule can be single-stranded or double-stranded.

In the case of IGSF11/domain modulator compounds being CRISPR/Cas9 constructs and/or guide RNA/DNAs (gRNA/gDNA) and/or tracrRNAs, the basic rules for the design of CRISPR/Cas9 mediated gene editing approaches are known to the skilled artisan and for example reviewed in Wiles M V et al (Mamm Genome 2015, 26:501) or in Savic N and Schwank G (Transl Res 2016, 168:15). Alternatively, gene-specific guide RNAs (gRNAs) useful to knockout the target gene using CRISPR/Cas9 technology can be designed using the online algorithm developed by the Broad Institute (https://portals.broadinstitute.org/gpp/public/analysis-tools/sgrna-design).

In particular embodiments, the IGSF11/domain modulator compounds may be an inhibitory nucleic acid molecule, such as antisense nucleotide molecule including a siRNA or shRNA molecule, for example as described in detail herein below.

A modulator (eg an inhibitor) compound of IGSF11/domain that is a nucleic acid can be, for example, an anti-sense nucleotide molecule, a RNA, DNA or PNA molecule, or an aptamer molecule. An anti-sense nucleotide molecule can, by virtue of it comprising an anti-sense nucleotide sequence, bind to a target nucleic acid molecule (eg based on sequence complementarity) within a cell and modulate the level of expression (transcription and/or translation) of IGSF11/domain, or it may modulate expression of another gene that controls the expression, function and/or stability of IGSF11/domain. Similarly, an RNA molecule, such as a catalytic ribozyme, can bind to and alter the expression of the IGSF11 gene, or it can bind to and alter the expression of other genes that control the expression, function and/or stability of IGSF11/domain, such as a transcription factor for or repressor protein of IGSF11/domain. An aptamer is a nucleic acid molecule that has a sequence that confers it an ability to form a three-dimensional structure capable of binding to a molecular target.

A modulator (eg an inhibitor) modulator compound of IGSF11/domain that is a nucleic acid can be, for example, can further be a double-stranded RNA molecule for use in RNA interference. RNA interference (RNAi) is a process of sequence-specific gene silencing by post-transcriptional RNA degradation or silencing (prevention of translation). RNAi is initiated by use of double-stranded RNA (dsRNA) that is homologous in sequence to the target gene to be silenced. A suitable double-stranded RNA (dsRNA) for RNAi contains sense and antisense strands of about 21 contiguous nucleotides corresponding to the gene to be targeted that form 19 RNA base pairs, leaving overhangs of two nucleotides at each 3′end (Elbashir et al., Nature 411:494-498 (2001); Bass, Nature 411:428-429 (2001); Zamore, Nat. Struct. Biol. 8:746-750 (2001)). dsRNAs of about 25-30 nucleotides have also been used successfully for RNAi (Karabinos et al., Proc. Natl. Acad. Sci. USA 98:7863-7868 (2001). dsRNA can be synthesised in vitro and introduced into a cell by methods known in the art.

A particularly preferred example of an antisense molecule of the invention is a small interfering RNA (siRNA) or endoribonuclease-prepared siRNA (esiRNA). An esiRNA is a mixture of siRNA oligos resulting from cleavage of a long double-stranded RNA (dsRNA) with an endoribonuclease such as Escherichia coli RNase III or dicer. esiRNAs are an alternative concept to the usage of chemically synthesised siRNA for RNA Interference (RNAi). An esiRNAs is the enzymatic digestion of a long double stranded RNA in vitro.

As described above, a modulator of the invention that is an RNAi molecule (such as an siRNA) may bind to and directly inhibit or antagonise the expression of mRNA of IGSF11 or the domain thereof. However, a modulator of the invention that is an RNAi molecule (such as an siRNA) may bind to and inhibit or antagonise the expression of mRNA of another gene that itself controls the expression (or function or stability) of IGSF11/domain. Such other genes may include transcription factors or repressor proteins of IGSF11 or such domain.

The sequence identity of the antisense molecule according to the invention in order to target a IGSF11/domain mRNA (or to target mRNA of a gene controlling expression, function and/or stability of IGSF11/domain), is with increasing preference at least 75%, at least 80%, at least 85%, at least 90%, at least 95%, at least 98%, at least 99% and 100% identity to a region of a sequence encoding the IGSF11/domain protein, as disclosed herein (or of such other controlling gene). Preferably, the region of sequence identity between the target gene and the modulating antisense molecule is the region of the target gene corresponding to the location and length of the modulating antisense molecule. For example, such a sequence identity over a region of about 19 to 21 bp of length corresponding to the modulating siRNA or shRNA molecule). Means and methods for determining sequence identity are known in the art. Preferably, the BLAST (Basic Local Alignment Search Tool) program is used for determining the sequence identity with regard to one or more IGSF11 RNAs as known in the art. On the other hand, preferred antisense molecules such as siRNAs and shRNAs of the present invention are preferably chemically synthesised using appropriately protected ribonucleoside phosphoramidites and a conventional RNA synthesiser. Suppliers of RNA synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK).

The ability of antisense molecules, siRNA, and shRNA to potently, but reversibly, silence genes in vivo make these molecules particularly well suited for use in the pharmaceutical composition of the invention which will be also described herein below. Ways of administering siRNA to humans are described in De Fougerolles et al., Current Opinion in Pharmacology, 2008, 8:280-285. Such ways are also suitable for administering other small RNA molecules like shRNA. Accordingly, such pharmaceutical compositions may be administered directly formulated as a saline, via liposome based and polymer-based nanoparticle approaches, as conjugated or complexation pharmaceutical compositions, or via viral delivery systems. Direct administration comprises injection into tissue, intranasal and intratracheal administration. Liposome based and polymer-based nanoparticle approaches comprise the cationic lipid Genzyme Lipid (GL) 67, cationic liposomes, chitosan nanoparticles and cationic cell penetrating peptides (CPPs). Conjugated or complexation pharmaceutical compositions comprise PEI-complexed antisense molecules, siRNA, shRNA or miRNA. Further, viral delivery systems comprise influenza virus envelopes and virosomes.

The antisense molecules, siRNAs, shRNAs may comprise modified nucleotides such as locked nucleic acids (LNAs). The ribose moiety of an LNA nucleotide is modified with an extra bridge connecting the 2′ oxygen and 4′ carbon. The bridge “locks” the ribose in the 3-endo (North) conformation, which is often found in the A-form duplexes. LNA nucleotides can be mixed with DNA or RNA residues in the oligonucleotide whenever desired. Such oligomers are synthesised chemically and are commercially available. The locked ribose conformation enhances base stacking and backbone pre-organisation. This significantly increases the hybridisation properties (melting temperature) of oligonucleotides. Particularly preferred example of siRNAs is GapmeR (LNA™ GapmeRs (Exiqon)). GapmeRs are potent antisense oligonucleotides used for highly efficient inhibition of IGSF11/domain mRNA (or of mRNA of a gene controlling expression, function and/or stability of IGSF11). GapmeRs contain a central stretch of DNA monomers flanked by blocks of LNAs. The GapmeRs are preferably 14-16 nucleotides in length and are optionally fully phosphorothioated. The DNA gap activates the RNAse H-mediated degradation of targeted RNAs and is also suitable to target transcripts directly in the nucleus.

Preferred antisense molecules for targeting IGSF11 (or for targeting the domain of IGSF11) are antisense molecules or constructs having a sequence complementary to a region (such as one described above) of a nucleic acid sequence of an IGSF11/domain mRNA, preferably a sequence complementary to a region of a sequence encoding the amino acid sequence shown in SEQ ID NOs: 371 to 373, more preferably, a sequence complementary to a region of between about 15 to 25 bp (such as between about 19 and 21 bp) of a sequence encoding the amino acid sequence shown in SEQ ID NO: 371 to 373, or of a sequence encoding the amino acid sequence shown in SEQ ID NO: 376, 388 or 399, or of a sequence encoding the amino acid sequence shown in SEQ ID NO: 375 or 389.

In particular embodiments, an antisense molecule for targeting IGSF11/domain may not be (or, alternatively, may be) one or more of an siRNA selected from the IGSF11 siRNA molecules identified as “s1”, “s2”, “s3, or “s4” herein (eg in Table A; SEQ ID NOs: 384, 385, 386 and 387, respectively).

TABLE A
exemplary siRNA sequences used

| | | | Order number | |
|---|---|---|---|---|
| | | | (Dharmacon/GE | SEQ |
| Gene | siRNA ID | siRNA sequence | Lifesciences) | ID NO. |
| IGSF11 | s1 | CAACAUACCAUCCAUUUAU | D-010688-01 | 384 |
| IGSF11 | s2 | GGAACGAAUUGGUGCAGUA | D-010688-02 | 385 |
| IGSF11 | S3 | GAACAUCAGUGCCCUGUCU | D-010688-03 | 386 |
| IGSF11 | S4 | CAGGAACAUUGGACUAAUA | D-010688-04 | 387 |
| IGSF11 | Pool | As above: s1, s2, s3 | As above | N/A |
| | | and s4 | | |
| PD-L1 | Smart Pool | | M-015836-01 | N/A |
| CEACAM6 | Smart Pool | | M-015306-01 | N/A |
| Control | siCtrl/siCtrl1 | | 4390844 | N/A |
| | | | (ThermoScientific) | |

In particular embodiments, an antisense molecule for targeting IGSF11/domain may not be (or, alternatively, may be) one or more of an shRNA molecule identified as “shIGSF11” herein (eg as may be purchased from Sigma-Aldrich, eg The RNAi Consortium (TRC) numbers: TRCN0000431895, TRCN0000428521 or TRCN0000425839 for IGSF11 CDS, and SHC002 for control shRNA).

In one embodiment, the antisense molecules of the invention may be isolated. In another embodiment, the antisense molecules of the invention may be recombinant, synthetic and/or modified, or in any other way non-natural or not a product of nature. For example, a nucleic acid of the invention may contain at least one nucleic acid substitution (or deletion) modification such as between 1 and about 5 such modifications, preferably no more than 1, 2 or 3 such modifications) relative to a product of nature, such as a human nucleic acid. As described above, the antisense molecules of the invention may be modified by use of non-natural nucleotides, or may be conjugated to another chemical moiety. For example, such chemical moieties may be a heterologous nucleic acid conferring increased stability or cell/nucleus penetration or targeting, or may be a non-nucleic acid chemical moiety conferring such properties, of may be a label.

Certain preferred embodiments pertain to a genetic construct for gene editing that is used as a modulator (eg an inhibitor) of expression, function and/or stability of IGSF11/domain in the context of the herein described invention. By using genome editing constructs it is possible to modulate the expression, stability and/or activity of IGSF11. Genome editing approaches are well known in the art and may be easily applied when the respective target genomic sequences are known. Preferably, such approaches may be used in gene therapy using e.g. viral vectors, which specifically target tumour cells in accordance with the above descriptions.

In case of genome editing, DNA is inserted, replaced, or removed, from a genome using artificially engineered nucleases, or so called “molecular scissors”. The nucleases create specific double-stranded break (DSBs) at desired locations in the genome, and harness the cell's endogenous mechanisms to repair the induced break by natural processes of homologous re-combination (HR) and non-homologous end-joining (NHEJ). For doing so, engineered nucleases such as zinc finger nucleases (ZFNs), Transcription Activator-Like Effector Nucleases (TALENs), the CRISPR/Cas system, and engineered meganuclease re-engineered homing endonucleases are routinely used for genome editing. According to another preferred embodiment, for genome editing approaches for modulating/inhibiting IGSF11, the rare-cutting endonuclease is Cas9, Cpfl, TALEN, ZFN, or a homing endonuclease may be used. Also, it may be convenient to engineer using DNA-guided Argonaute interference systems (DAIS). Basically, said Argonaute (Ago) protein is heterologously expressed from a polynucleotide introduced into said cell in the presence of at least one exogenous oligonucleotide (DNA guide) providing specificity of cleavage to said Ago protein to a preselected locus. The TALEN and Cas9 systems are respectively described in WO 2013/176915 and WO 2014/191128. The Zinc-finger nucleases (ZFNs) are initially described in Kim, Y G; Cha, J.; Chandrasegaran, S. (“Hybrid restriction enzymes: zinc finger fusions to Fok I cleavage domain” (1996). Proc Natl Acad Sci USA 93 (3): 1156-60). Cpfl is class 2 CRISPR Cas System described by Zhang et al. (Cpfl is a single RNA-guided Endonuclease of a Class 2 CRIPR-Cas System (2015) Cell 163:759). The argonaute (AGO) gene family was initially described in Guo S, Kemphues K J. (“par-1, a gene required for establishing polarity in C. elegans embryos, encodes a putative Ser/Thr kinase that is asymmetrically distributed” (1995) Cell 81:611).

The use of the CRISPR/Cas9, CRISPR/Cpfl or the Argonaute genome-editing systems is particularly adapted to be used in combination with the transfection of guide RNA or guide DNA sequences. In this context the guide-RNAs and a nucleic acid sequence coding for Cas9 nickase (or similar enzymes), is transfected into a target cell (preferably a tumour cell) so that they form a complex able to induce a nick event in double-stranded nucleic acid targets in order to cleave the genetic sequence between said nucleic acid targets.

In certain embodiments, it may be useful to deliver the guide RNA-nanoparticle formulations separately from the Cas9. In such an instance, a dual-delivery system is provided such that the Cas9 may be delivered via a vector and the guide RNA is provided in a nanoparticle formulation, where vectors are considered in the broadest sense simply as any means of delivery, rather than specifically viral vectors. Separate delivery of the guide RNA-nanoparticle formulation and the Cas9 may be sequential, for example, first Cas9 vector is delivered via a vector system followed by delivery of sgRNA-nanoparticle formulation) or the sgRNA-nanoparticle formulation and Cas9 may be delivered substantially contemporaneously (i.e., co-delivery). Sequential delivery may be done at separate points in time, separated by days, weeks or even months. In certain embodiments, multiple guide RNAs formulated in one or more delivery vehicles (e.g., where some guide RNAs are provided in a vector and others are formulated in nanoparticles) may be provided with a Cas9 delivery system. In certain embodiments, the Cas9 is also delivered in a nanoparticle formulation. In such an instance, the guide RNA-nanoparticle formulation and the Cas9 nanoparticle formulation may be delivered separately or may be delivered substantially contemporaneously (i.e., co-delivery). As will now be apparent to the person of ordinary skill, the gene target of such genome-editing approaches may be the gene of IGSF11, or that part of the gene encoding the IgC2 (or IgV) domain of IGSF11. Alternatively, the gene target of such editing may be another gene that controls the expression, function and/or stability of IGSF11/domain, for example a transcription factor for or repressor protein of IGSF11/domain.

In preferred embodiments of the invention, the compounds for genome editing approaches according to the invention comprise at least the use of a guide RNA or DNA complementary to a region (such as one described above) of a IGSF11/domain sequence. In some additional embodiments, the compounds for use in genome editing approaches of the invention may include donor sequences homologous to such a region of IGSF11/domain, as templates for homology directed repair. The donor sequences comprise a mutated sequence of IGSF11/domain that when used in the CRISPR induced repair mechanism in a target cell, is by homologous recombination inserted/copied into the IGSF11 genomic locus or that part of the genomic locus encoding an IgC2 (or IgV) domain of IGSF11, and therefore yields into a mutated IGSF11 gene which is characterised by a reduced expression, function and/or stability of the expressed IGSF11 or such domain. CRISPR/Cas9 genome editing in cancer therapy is reviewed for ex-ample in Khan F A et al: “CRISPR/Cas9 therapeutics: a cure for cancer and other genetic diseases.” (Oncotarget. 2016 May 26. doi: 10.18632/oncotarget.9646; incorporated by reference in its entirety).

Hetero-Bi-Functional Modulating Compounds

In particular embodiments, modulating (eg inhibiting) compounds of IGSF11 (or of an IgC2 (or IgV) domain of IGSF11) may be a hetero-bi-functional compound that contains two ligands connected by a linker, wherein one ligand binds to the target protein (in this case, IGSF11/domain or a gene that controls the expression, amount, function, activity and/or stability of IGSF11/domain) and the other ligand binds to and/or recruits a component of the cellular protein degradation machinery such as binding to a ubiquitin ligase protein (eg E3 ubiquitin ligase) or such as recruiting a chaperone protein. Examples of such hetero-bi-functional compounds include PROTACs (“PROteolysis TAgeting Chimera) or HyT (“hydrophobic tagging”) molecules, in each case designed to bind to the target protein for the present invention. The general principles of PROTACs and HyT molecules are reviewed in Huang & Dixit 2016 (Cell Research 26:484) and exemplified specifically in, for example, WO 2016/146985A1.

A PROTAC that binds to the target protein (eg IGSF11/domain) with one ligand and with the other ligand to an E3 ubiquitin ligase protein thereby brings the ligase and the target into close proximity. Without being bound by any particular theory it is generally understood that it is this close proximity which in turn triggers the poly-ubiquitination and subsequent proteasome-dependent degradation of the target protein of interest. Supporting evidence for a PROTAC approach on a general level is provided by known proof-of-concept examples where alternative PROTACs have been used to degrade: the Estrogen receptor (Cyrus et al 2010, Chem Bio Chem 11:1531); the Androgen-receptor (Sakamoto et al 2003, Mol Cell Proteomics 2:1350); methionine aminopeptidease-2 (Sakamoto et al 2001, PNAS 98:8554); as well as the Aryl Hydrocarbon Receptor (Lee et al 2007, Chem Bio Chem 8:2058).

The concept of hydrophobic tagging is similar to that of PROTAC, but instead of using a ligand to recruit a specific E3 ligase, a synthetic hydrophobic group, such as adamantane, linked to a chemical moiety that specifically recognizes the target protein (eg IGSF11/domain), assumes the role of “recruiter” for the degradation machinery. Upon binding to the target protein, the hydrophobic tag mimics or induces a misfolded state. Without being bound by any particular theory it is generally understood that modification of the target protein with a bulky hydrophobic side-group attracts the chaperone machinery, the primary goal of which is to help refold misfolded proteins. Since the covalent modification cannot be easily removed, the target protein remains unfolded and is eventually cleared by ubiquitin-proteasome mediated degradation.

IGSF11 Modulating Uses, Medical Uses and Methods of Treatment

Modulating compounds of IGSF11 (VSIG3) or of an IgC2 (or IgV) domain of IGSF11, or of a variant thereof, and/or the ABPs, NAC, (host) cells and the pharmaceutical compositions of the invention can be used in various ways to modulate the expression, function, activity and/or stability of the IGSF11/domain (or variant thereof), including their use in therapy or for prophylaxis.

Accordingly, in a further aspect, herein provided is a method of modulating the expression, function, activity and/or stability of IGSF11 (VSIG3) or of an IgC2 domain of IGSF11 (or, of an IgV domain of IGSF11), or of a variant of IGSF11 comprising contacting a cell that expresses said IGSF11, domain or variant with a modulating compound as described above, in particular an ABP of the invention or an NAC encoding said ABP. When such ABP is a modulator of the expression, function, activity and/or stability of said IGSF11, domain or variant, thereby the expression, function, activity and/or stability of said IGSF11, domain or variant is modulated. Such method may be practiced on cells that are present ex-vivo, that is where said cells are contained in receptacles or containers, such as those used in research facilities. Accordingly, in such embodiments such method of the invention can be described as an in-vitro method of modulating the expression, function, activity and/or stability of IGSF11 (VSIG3) or of an IgC2 (or IgV) domain of IGSF11, or of a variant thereof. However, in alternative embodiments, the method may be practiced using cells within the body, for example an in-vivo method of modulating the expression, function, activity and/or stability of IGSF11 (VSIG3) or of an IgC2 (or IgV) domain of IGSF11, or of a variant thereof.

In particular of such embodiments, such an in-vitro (or in-vivo) method comprises the inhibition of the function and/or activity of the IGSF11, domain or variant, when such modulating compound (eg the ABP) is an inhibitor of and/or antagonist of such function and/or activity. In some embodiments of such method, it further comprises the step of contacting the cell with an immune cell, such as a CTL or TIL. Preferably, the ABP is an antibody, or an antibody fragment, and is an inhibitor or antagonist of the function and/or activity of the IGSF11, domain or variant.

In particular of such embodiments, such an in-vitro (or in-vivo) method comprises the activation of the function and/or activity of the IGSF11, domain or variant, when such modulating compound (eg the ABP) is an activator of and/or agonist of such function and/or activity. In some embodiments of such method, it further comprises the step of contacting the cell with an immune cell, such as a CTL or TIL. Preferably, the ABP is an antibody, or an antibody fragment, and is an activator and/or agonist of the function and/or activity of the IGSF11, domain or variant. In certain embodiments of these aspects, the method of modulating comprises contacting a cell that expresses said IGSF11 or variant with a modulating compound as described above that is an activator and/or agonist of the function and/or activity of the IGSF11, domain or variant, and the method mediates any one or combination of at least one of the functional characteristic or effects of the activating or agonistic modulators described herein, in particular as set forth in the section above “Modulators of IGSF11 expression, function, activity and/or stability.

In other certain embodiments of these aspects, the method of modulating comprises contacting a cell that expresses said IGSF11, domain or variant with a modulating compound as described above that is an inhibitor and/or antagonist of the function and/or activity of the IGSF11, domain or variant, and the method mediates any one or combination of at least one of the functional characteristic or effects of the inhibitor or antagonist modulators described herein, in particular as set forth in the section above “Modulators of IGSF11 expression, function, activity and/or stability.

In particular embodiments, the modulating compound (in particular, an ABP) is an inhibitor and/or antagonist of the function and/or activity of the IGSF11, domain or variant and inhibits the interaction between an interacting protein (such as of VSIR (VISTA) protein or a variant thereof) and IGSF11 protein or of an IgC2 (or IgV) domain of IGSF11, or a variant thereof; that is, such a compound inhibits the binding function and/or activity of the IGSF11 protein, domain or variant thereof.

In preferred embodiments of the therapeutic aspects, the modulating compound (such as one that is an inhibitor or antagonist of expression, function, activity and/or stability of the IGSF11, domain or variant) for example an ABP, or an NAC encoding said ABP, is capable of: (i) modulating the expression, function, activity and/or stability of the IGSF11, domain or variant; and/or (ii) enhancing a cell-mediated immune response to a mammalian cell, decreases or reduces the resistance of cells (such as tumour cells that express the IGSF11, domain or variant), to an immune response. In other certain preferred embodiments of the invention, the ABP (such as one that is an inhibitor or antagonist of expression, function, activity and/or stability of the IGSF11, domain or variant, in particular one that inhibits the binding function and/or activity of the IGSF11 protein or variant thereof to an interacting protein (eg, VSIR (VISTA) protein or a variant thereof)), or an NAC encoding said ABP, enhances or increases the sensitivity of cells (such as tumour cells that express the IGSF11, domain or variant), to an immune response.

In one embodiment, the compound is not an ABP that is the subject of one or more of the provisos (A), (B), (C), (D), (E) and/or (F) as set out elsewhere herein

The term “resistance” refers to an acquired or natural resistance of a cell involved with (eg of or affected by) a disease (eg a proliferative disorder), such as tumour or cancer cell, to a patient's own immune response (such as a cell-mediated immune response), or to immune responses aided by immune therapy such as adoptive T-cell transfer or treatment with checkpoint blockers. Therefore, a resistant cell (eg a resistant tumour or cancer cell) is more likely to escape and survive humoural and/or cellular immune defence mechanisms in a subject having the disorder (such as the tumour or cancer). A treatment of a resistant proliferative disease, such as tumour/cancer resistance, in context of the invention shall be effective if, compared to a non-treated control, the cell involved with the proliferative disease (such as a cell of the tumour of cancer) becomes more sensitive or susceptible to an immune response (such as a cell-mediated immune response)—in other words will be more likely to be recognised and/or neutralised (for example by cytotoxic processes such as apoptosis) by the subject's immune response.

Accordingly, in particular embodiments of the invention, cell(s) involved with the disease may be resistant against (to) a cell-mediated immune response; and/or such cell(s) may have or display a resistant phenotype.

In preferred embodiments of the invention, the terms “cellular resistance”, “cell resistance” and the like refers to a resistance of the subject cell(s) (such as a tumour or cancer cell) to a cell-mediated immune response, such as a cytotoxic T lymphocyte (CTL) response (eg, the tumour or tumour cell being nonresponsive to, or having reduced or limited response to a CTL targeting a tumour cell). A tumour cell may show a reduced or limited response when contacted with a CTL specific for an antigen expressed on that tumour cell. A reduced or limited response is a reduction to a 90% cytotoxic T cell response, preferably a reduction to 80%, 70%, 60%, 50% or more preferably a reduction to 40%, 30%, 20% or even less. In this case, 100% would denote the state wherein the CTLs can kill all of the subject cells involved with the proliferative disorder in a sample. Whether or not a subject cell (eg a tumour cell) is resistant to a patient's (cell-mediated) immune response may be tested in-vitro by contacting a sample of the subjects such cells (eg autologous tumour cells) with (eg autologous) T-cells and thereafter quantifying the survival/proliferation rate of the (eg) tumour cells. As an alternative, the reduction in (cell-mediated) immune response is determined by comparing cancer samples of the same cancer before and after the resistance is acquired (for example induced by therapy), or by comparing with a cancer sample derived from a different cancer which is known to have no resistance to the CTL. On the other hand, the treatments of the present invention include the sensitisation of cells involved with the proliferative disorder against CTL and therefor to decrease resistance of such cells. A decrease of (eg tumour) cell resistance against CTL is preferably a significant increase of CTL toxicity, preferably a 10% increase, more preferably 20%, 30%, 40%, 50%, 60%, 70%, 80% or more, even more preferably 2 fold increase, 3 fold, 4 fold, 5 fold or more.

In particular embodiments, a resistant phenotype of the cells involved with the proliferative disorder is displayed by such cells when a subject suffering from the proliferative disorder (eg a cancer or tumour) has been previously treated with an (immune)therapy and, for example, such proliferative disorders has progressed despite such prior (immune)therapy. For example, a class of subject suitable for the various therapeutic methods of the invention can be those whose tumour (or cancer) has progressed (such as has relapsed or recurred, or has not responded to) after prior treatment with a cancer immunotherapy. In certain embodiments, such prior treatment may be any immunotherapy as described elsewhere herein, including adoptive immune cell transfer (eg TCR or CAR T cell therapy), an anti-tumour vaccine, an antibody binding to an immune checkpoint molecule (such as CTLA-4, PD-1 or PD-Li). In other embodiments, the subject may suffer from a tumour or cancer, and such cancer may have progressed (such as has relapsed or recurred, or has not responded to) after prior radiotherapy.

The immune response, is, in particular of such embodiments, a cell-mediated immune response such as one mediated by T-cells including cytotoxic T-cells and/or TILs; and/or the immune response is the lysis and/or killing of the cells, in particular those that express IGSF11, an IgC2 (or an IgV) domain of IGSF11, or a variant thereof) that is mediated by cytotoxic T-cells and/or TILs. In other particular of such embodiments, the immune response is a cytotoxic immune response against cells (such as tumour cells and/or cells the IGSF11, domain or variant), in particular a cell-mediated cytotoxic immune response such as one mediated by T-cells including cytotoxic T-cells and/or TILs.

Specifically, in certain preferred embodiments of such therapeutic aspects the modulating compound as disclosed herein, in particular the ABP (such as one that is an inhibitor or antagonist of expression, function, activity and/or stability of the IGSF11, domain or variant thereof), or an NAC encoding said ABP, enhances or increases killing and/or lysis of cells expressing IGSF11 or an IgC2 (or an IgV) domain of IGSF11, or variant thereof, (such as tumour cells); preferably killing and/or lysis being mediated by cytotoxic T-cells and/or TILs, and/or mediated by an enhancement of or increase in the sensitivity of the cells expressing the IGSF11, domain or variant thereof to a (cytotoxic) immune response, such an immune response described above, and/or mediated by a decrease in or reduction of the resistance of the cells expressing the IGSF11, domain or variant thereof to a (cytotoxic) immune response, such an immune response described above.

The cells that express IGSF11 or an IgC2 (or an IgV) domain of IGSF11, or variant thereof are, in certain of such preferred embodiments, cancer cells or are cells that originated from a tumour cell. Exemplary cancer or tumour cells can be those as described or exemplified elsewhere herein.

Also specifically, in alternative or additional certain preferred embodiments of such therapeutic aspects the modulating compound and/or the ABP as disclosed herein (such as one that is an inhibitor or antagonist of expression, function, activity and/or stability of the IGSF11, domain or variant thereof), or an NAC encoding said ABP, enhances or increases killing and/or lysis of tumour cells (eg, is capable of and/or is able to enhance or increase killing and/or lysis of tumour cells), preferably cancer cells or cells that originate from a tumour, and/or cells expressing IGSF11 or an IgC2 (or an IgV) domain of IGSF11, or variant thereof. Such killing and/or lysis being may be, in certain embodiments, mediated by cytotoxic T-cells (including in some embodiments CAR-T cells, eg autologous T cells expressing an ABP of the invention as chimeric antigen receptor) and/or TILs, and/or mediated by an enhancement of or increase in the sensitivity of the cells to a (cytotoxic) immune response, such an immune response described above, and/or mediated by a decrease in or reduction of the resistance of the tumour cells thereof to a (cytotoxic) immune response, such an immune response described above.

In another further and/or alternative embodiment, preferred embodiments of such therapeutic aspects the modulating compound and/or the ABP as disclosed herein (such as one that is an inhibitor or antagonist of expression, function, activity and/or stability of the IGSF11, domain or variant thereof), or an NAC encoding said ABP, is an anti-tumour compound (such as an anti-tumour ABP or antibody). For example, such a compound (eg, an anti-tumour ABP or an anti-tumour antibody) inhibits the growth of a tumour in-vivo (eg, is capable of and/or is able to inhibit the growth of a tumour in-vivo). Suitable experimental (in-vivo) models of cancer (eg, murine models of cancer) are known to the person of ordinary skill, and include those described herein (eg, in Example A) and/or are readily accessible from contract research organisations such as Charles River Laboratories. Following the disclosure herein, such person or ordinary skill will be able to utilise such (in-vivo) models of cancer to identify a modulating compound and/or the ABP as disclosed herein (such as one that is an inhibitor or antagonist of expression, function, activity and/or stability of the IGSF11, domain or variant thereof), or an NAC encoding said ABP, that has (or that is capable of and/or is able to exhibit) such anti-tumour properties, and/or that is capable of inhibiting (ie, inhibits) growth of a tumour in-vivo.

The inventors describe herein that, surprisingly, ABPs of the invention (eg, those that specifically bind to the IgC2 domain (or to the IgV domain) of IGSF11) exhibit tumour growth inhibition when administered to mice in one or more murine models of cancer.

In other certain preferred embodiments of such therapeutic aspects, the modulating compounds, in particular the ABP (such as one that is an inhibitor or antagonist of expression, function, activity and/or stability of IGSF11 or variant thereof, in particular that is an inhibitor of the VSIR-binding function of the IGSF11, domain or variant), or an NAC encoding said ABP, increases T-cell activity and/or survival (and/or increases T-cell proliferation), which in certain embodiments, may lead to an enhancement of a (cytotoxic) immune response mediated by such T-cells.

Lines et al (2014) described that T cells both express and respond to VSIR (VISTA), indicating that T cells and/or other components of the immune system, in certain circumstances, may express IGSF11 (eg, acting as a receptor for VSIR). Without being bound to theory, FIG. 1 of Lines et al 2014, indicates that in the tumour microenvironment (TME) the VSIR receptor (“VISAT-R”, eg, now to include IGSF11) may also be expressed on CTLs. The present inventors hypothesise that (analogous to VSIR expression being found on various types of immune cells, as described above) IGSF11 may be expressed on other cells involved in the regulation of the immune response (eg, IGSF11 expression by monocytes and/or Tregs), and the immune regulatory effects mediated by the IGSF11-VSIR axis between immune cells may also lead to a reduction in the (eg anti-tumour) immune response (eg a cell-mediated immune response), manifesting itself in a “resistance” of cells involved in a disease to a cell-mediated immune response. Such an inter-immune cell IGSF11-VSIR axis may play a role at the site of the tumour, for example in the TME (eg tumour bed) between eg VSIR-expressing T cells and IGSF11-expressing monocytes or Tregs that are present at or associated with the site of the tumour, or may play a role outside of the TME, such as in one or more component of the peripheral immune system, in particular by the expression of IGSF11 on monocytes driving T cell suppression in lymph nodes. In particular, the presence of tumour associated macrophages (TAMs) in cancers is associated with poor prognosis, as they are believed to facilitate tumour invasions, angiogenesis and metastasis, and “subvert” the adaptive immune response potentially via their T cell recruitment/activation ability. (Wlliams et al, 2016; NPJ Breast Cancer, 2:15025; Nielsen & Schmid, 2017; Mediators of Inflammation Article ID 9624760). Accordingly, inhibition of the IGSF11-VSIR interaction (eg, by compounds or ABPs of the present invention) in particular where both IGSF11 and VSIR are being expressed by different components (eg different cell types) of a cellular immune response—can also lead to attenuation of the inhibition of the immune response mediated by such an IGSF11-VSIR interaction. Therefore, it is also envisioned by the present invention, those embodiments where IGSF11 is expressed by cells other than eg cancer cells. In particular by immune cells such as monocytes (eg, see Comparative Example 6) or Tregs. For example, the cells described herein as being “associated with” the disease, disorder or condition, includes not only eg cancer cells (being directly involved in a proliferative disorder), but may also include non-cancer cells, eg regulatory immune cells which may be involved in the (over) inhibition of eg T cell activation, such as monocytes and/or Tregs (either inside, or outside of, the TME) but hence such regulatory immune cells are therefore indirectly associated with the development or (or lack of response of) a proliferative disorder to a cellular immune response. The present inventors also hypothesise that given the potential for a role of the IGSF11-VSIR axis in regulation of the immune system, and in particular by expression of IGSF11 (VSIG3) on immune cells (such as T cells) or monocytes, that IGSF11 (VSIG3) expression, or expression of an IgC2 (or an IgV) domain of IGSF11, on T cells or monocytes can be immunosuppressive by interacting with VSIR (VISTA) present on other immune cells. Accordingly, that an activator or agonist of IGSF11 (VSIG3) or of an IgC2 (of IgV) domain of IGSF11, will have utility as an immune suppression agent, and hence suitable for the treatment of diseases, disorders or conditions associated with an over-active immune system or an immune system displaying undesired activity, such as autoimmunity, allergy or inflammatory conditions, in particular for the treatment or prevention of allergy, autoimmunity, transplant rejection, inflammation, graft vs host disease or sepsis (or a condition associated with such diseases, disorders or conditions).

Accordingly, in a fifth aspect, the invention relates to a method for the treatment of a disease, disorder or condition in a mammalian subject by administering a product to the subject wherein the product is an IGSF/domain binder and/or is a modulator of the expression, function, activity and/or stability of immunoglobulin superfamily member 11 (IGSF11, or VSIG3) or of an IgC2 domain of IGSF11 (or, in another aspect, of an IgV domain of IGSF11), or of a variant thereof. In a related aspect, the invention relates to a product for use in medicine, wherein the product is a compound that is an IGSF/domain binder and/or is modulator of the expression, function, activity and/or stability of immunoglobulin superfamily member 11 (IGSF11, or VSIG3) or of an IgC2 domain of IGSF11 (or, in another aspect, of an IgV domain of IGSF11), or of a variant thereof. In particular embodiments, of these medical/treatment claims, the modulating compound (such as an ABP) is an inhibitor of the function of binding to an interacting protein (such as, VSIR-binding) of the IGSF11, domain or variant thereof; and/or wherein the product is selected from the list consisting of an ABP, ABD, nucleic acid, NAC or recombinant host cell of the invention, in particular an ABP of the invention.

In a related aspect, the invention also relates to method of treating or preventing a disease, disorder or condition in a mammalian subject in need thereof, comprising administering to said subject at least once an effective amount of modulating compound as desired above, or, and in particular administering to said subject at least once an effective amount of the ABP, the NAC, the (host) cells, or the pharmaceutical composition as described above.

In another related aspect, the invention also relates to the use of a product of the invention as describe above, or a modulating compound as described above (in particular an ABP of the invention) for the manufacture of a medicament, in particular for the treatment of a disease, disorder or condition in a mammalian subject, in particular where the disease, disorder or condition is one as set out herein.

The term “treatment” in the present invention is meant to include therapy, e.g. therapeutic treatment, as well as prophylactic or suppressive measures for a disease (or disorder or condition). Thus, for example, successful administration of an IGSF11 inhibitor (or of an inhibitor of an IgC2 (or IgV) domain of IGSF11) prior to onset of the disease results in treatment of the disease. “Treatment” also encompasses administration of an IGSF11 inhibitor (or of an inhibitor of an IgC2 (or IgV) domain of IGSF11) after the appearance of the disease in order to ameliorate or eradicate the disease (or symptoms thereof). Administration of an IGSF11 inhibitor (or of an inhibitor of an IgC2 (or IgV) domain of IGSF11) after onset and after clinical symptoms, with possible abatement of clinical symptoms and perhaps amelioration of the disease, also comprises treatment of the disease. Those “in need of treatment” include subjects (such as a human subject) already having the disease, disorder or condition, as well as those prone to or suspected of having the disease, disorder or condition, including those in which the disease, disorder or condition is to be prevented.

In particular embodiments of these aspects, the modulating compound is one described above, and/or is an ABP, NAC, a (host) cell, or a pharmaceutical composition of the present invention; in particular is an ABP of the invention, and/or is an inhibitory nucleic acid of the invention.

Such a compound can for example in preferred embodiments, be an inhibitor or antagonist of expression, function, activity and/or stability of IGSF11 or of an IgC2 (or IgV) domain of IGSF11, or of the variant thereof. In particular, the compound inhibits the binding of an interacting protein (such as VSIR protein or a variant thereof) to IGSF11 protein or to an IgC2 (or IgV) domain of IGSF11 (or a variant thereof), in particular inhibits the binding of human VSIR protein (or a variant thereof) to human IGSF11 protein or IgC2 (or IgV) domain of human IGSF11 (or a variant thereof), such as inhibits the binding between the ECDs of such proteins; preferably wherein such proteins (or variants) and the inhibitions is described as above.

Such a compound can, for example, be a compound (such as an ABP or inhibitors nucleic acid) that enhances killing and/or lysis of cells expressing IGSF11, or an IgC2 (or IgV) domain of IGSF11 or a variant thereof, by cytotoxic T-cells and/or TILs.

In other aspects described elsewhere herein, are provided methods to detect and/or diagnose a disease, disorder or condition in a mammalian subject.

In one particular embodiment, the disease, disorder or condition that is characterised by a pathological immune response.

In a further particular embodiment, the disease, disorder or condition is characterised by expression of IGSF11 or of an IgC2 (or IgV) domain of IGSF11, or a variant thereof, in particular by expression of the IGSF11, domain or a variant thereof by cells associated with the disease, disorder or condition, such as cancer cells. For example, the disease, disorder or condition can be associated with the undesired presence of IGSF11-positive cells or cells positive for an IgC2 (or IgV) domain of IGSF11, or a variant thereof and or VSIR positive immune cells, in particular VSIR positive monocytes and/or macrophages (in particular, TAMs).

In a yet further particular embodiment, a subject suffering from, or suspected of suffering from, a disease, disorder or condition is characterised as: (i) having an IGSF11 positive cancer or a cancer positive for an IgC2 (or IgV) domain of IGSF11, or a variant thereof, and/or (ii) having VSIR positive immune cells, in particular VSIR positive monocytes and/or macrophages; and/or (iii) having IGSF11 positive immune cells, in particular IGSF11/domain positive monocytes (or Tregs); preferably wherein such IGSF11/domain positive immune cells are present at or associated with the site of a cancer or tumour (such as being present in the tumour bed or tumour micro environment (TME) of such cancer or tumour, in particular with the presence of TAMs and/or MDSCs).

A disorder, disorder or condition treatable by the subject matter of the invention is, in certain alterative embodiments, one characterised by expression of IGSF11 or of an IgC2 (or IgV) domain of IGSF11, or a variant thereof; in particular, one characterised by such expression that is aberrant, for example over- (or under-) expression or representation or activity of IGSF11/domain (in particular of phosphorylated IGSF117domain) in a given cell or tissue (such as those cells or tissues involved with the proliferative disease of the subject) compared to that in a healthy subject or a normal cell.

In yet a further particular embodiment, the disease, disorder or condition is characterised by expression and/or activity of IGSF11 or of an IgC2 (or IgV) domain of IGSF11, or a variant thereof, in particular such cells express mRNA and/or protein of IGSF11/domain, and/or are positive for such IGSF11, domain or variant thereof expression and/or activity.

In another particular embodiment, the disease, disorder or condition is a proliferative disorder (or a condition associated with such disorder or disease), in particular when the product or modulating compound (such as a ABP, ABD, nucleic acid, NAC or recombinant host cell of the invention, in particular an ABP of the invention) is an inhibitor and/or antagonist of the expression, function, activity and/or stability of IGSF11 (VSIG3), or of an IgC2 (or IgV) domain of IGSF11, or a variant thereof.

A “proliferative disorder” refers to a disorder characterised by abnormal proliferation of cells. A proliferative disorder does not imply any limitation with respect to the rate of cell growth, but merely indicates loss of normal controls that affect growth and cell division. Thus, in some embodiments, cells of a proliferative disorder can have the same cell division rates as normal cells but do not respond to signals that limit such growth. Within the ambit of “proliferative disorder” is neoplasm or tumour, which is an abnormal growth of tissue or cells. Cancer is art understood, and includes any of various malignant neoplasms characterised by the proliferation of cells that have the capability to invade surrounding tissue and/or metastasise to new colonisation sites. Proliferative disorders include cancer, atherosclerosis, rheumatoid arthritis, idiopathic pulmonary fibrosis and cirrhosis of the liver. Non-cancerous proliferative disorders also include hyperproliferation of cells in the skin such as psoriasis and its varied clinical forms, Reiter's syndrome, Pityriasis rubra pilaris, and hyperproliferative variants of disorders of keratinization (e.g., actinic keratosis, senile keratosis), scleroderma, and the like.

In more particular embodiments, the proliferative disorder is a cancer or tumour, in particular a solid tumour (or a condition associated with such cancer or tumour). Such proliferative disorders include, but are not limited to, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumour of the kidney, Ewing Sarcoma, chondrosarcoma, any haemotological malignancy (e.g., chronic lymphoblastic leukemia, chronic myelomonocytic leukemia, acute lymphoblastic leukemia, acute lymphocytic leukemia, acute myelogenous leukemia, acute myeloblasts leukemia, chronic myeloblastic leukemia, Hodgkin's disease, non-Hodgkin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, marginal zone lymphoma, Burkitt Lymphoma, mycosis fungoides, seary syndrome, cutaneous T-cell lymphoma, peripheral T cell lymphoma, chronic myeloproliferative disorders, myelofibrosis, myeloid metaplasia, systemic mastocytosis), and central nervous system tumors (e.g., brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma), myeloproliferative disorders (e.g., polycythemia vera, thrombocythemia, idiopathic myelofibrosis), soft tissue sarcoma, thyroid cancer, endometrial cancer, carcinoid cancer, or liver cancer.

In one preferred embodiment, the various aspects of the invention relate to, for example the ABPs of the invention used to detect/diagnose, prevent and/or treat, such proliferative disorders that include but are not limited to carcinoma (including breast cancer, prostate cancer, gastric cancer, lung cancer, colorectal and/or colon cancer, hepatocellular carcinoma, melanoma), lymphoma (including non-Hodgkin's lymphoma and mycosis fungoides), leukemia, sarcoma, mesothelioma, brain cancer (including glioma), germinoma (including testicular cancer and ovarian cancer), choriocarcinoma, renal cancer, pancreatic cancer, thyroid cancer, head and neck cancer, endometrial cancer, cervical cancer, bladder cancer, or stomach cancer.

Accordingly, in a preferred embodiment, the proliferative disease is a cancer, for example lung cancer, breast cancer, colorectal cancer, gastric cancer, hepatocellular carcinoma, pancreatic cancer, ovarian cancer, melanoma, myeloma, kidney cancer, head and neck cancer, Hodgkin lymphoma, bladder cancer or prostate cancer, in particular one selected from the list consisting of: melanoma, lung cancer (such as non-small cell lung cancer), bladder cancer (such as urothelial carcinoma), kidney cancer (such as renal cell carcinoma), head and neck cancer (such as squamous cell cancer of the head and neck) and Hodgkin lymphoma. Preferably, the proliferative disease is melanoma, or lung cancer (such as non-small cell lung cancer). Most preferably (eg, see Example B), the proliferative disease is a cancer selected from one of the group consisting of: lung cancer (in particular, squamous lung cancer), melanoma, head and neck squamous cell carcinoma (HNSCC), bladder cancer, thymoma and ovarian cancer.

In a particularly preferred embodiment, the disease, disorder or condition is a IGSF11-positive cancer or a cancer positive for the IgC2 (or IgV) domain of IGSF11, or variant thereof, and/or is a cancer characterised by the presence of VSIR positive immune cells, in particular VSIR positive monocytes and/or macrophages (in particular, TAMs) and/or is a cancer (or other proliferative disorder) characterised by being resistant and/or refractory to blockade of an immune checkpoint molecule (eg resistant and/or refractory to therapy for blockade of an immune checkpoint molecule), such as blockade using a ligand to an immune checkpoint molecule (as further described below, such as blockade of PD1/PDL1 and/or CTLA4; analogous to Gao et al, 2017). For example, in one such embodiment, the disease, disorder or condition can be a proliferative disorder (such as cancer) resistant and/or refractory to PD1/PDL1 and/or CTLA4 blockade therapy.

In a further particular embodiment, the disease, disorder or condition is an infectious disease (or a condition associated with such disorder or disease), in particular when the product or modulating compound (such as a ABP, ABD, nucleic acid, NAC or recombinant host cell of the invention, in particular an ABP of the invention) is an inhibitor and/or antagonist of the expression, function, activity and/or stability of IGSF11 (VSIG3) or an IgC2 (or IgV) domain of IGSF11, or variant thereof.

The term “infectious disease” is art recognised, and as used herein includes those diseases, disorders or conditions associated with (eg resulting from or caused by) by any pathogen or agent that infects mammalian cells, preferable human cells. Examples of such pathogens include bacteria, yeast, fungi, protozoans, mycoplasma, viruses, prions, and parasites. Examples of infectious disease include (a) viral diseases such as, for example, diseases resulting from infection by an adenovirus, a herpesvirus (e.g., HSV-I, HSV-II, CMV, or VZV), a poxvirus (e˜g-, an orthopoxvirus such as variola or vaccinia, or molluscum contagiosum), a picornavirus (e.g., rhinovirus or enterovirus), an orthomyxovirus (e.g., influenza virus), a paramyxovirus (e.g., parainfluenza virus, mumps virus, measles virus, and respiratory syncytial virus (RSV)), a cononavirus (e.g., SARS), a papovavirus (e.g., papillomaviruses, such as those that cause genital warts, common warts, or plantar warts), a hepadnavirus (e.g., hepatitis B virus), a flavi virus (e.g., hepatitis C virus or Dengue virus), or a retrovirus (e.g., a lentivirus such as HIV); (b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella; (c) other infectious diseases, such chlamydia, fungal diseases including but not limited to candidiasis, aspergillosis, histoplasmosis, cryptococcal meningitis, parasitic diseases including but not limited to malaria, Pneumocystis camii pneumonia, leishmaniasis, cryptosporidiosis, toxoplasmosis, and trypanosome infection and prions that cause human disease such as Creutzfeldt-Jakob Disease (CJD), variant Creutzfeldt-Jakob Disease (vCJD), Gerstmann-Straeussler-Scheinker syndrome, Fatal Familial Insomnia and kuru.

In yet another particular embodiment, the disease, disorder or condition is one associated with an over-active or immune system or an immune system displaying undesired activity, such as autoimmunity, allergy or inflammatory conditions, in particular for allergy, autoimmunity, transplant rejection, inflammation, graft vs host disease or sepsis (or a condition associated with such diseases, disorders or conditions), in particular when the product or modulating compound (such as a ABP, ABD, nucleic acid, NAC or recombinant host cell of the invention, in particular an ABP of the invention) is an activator and/or agonist of the expression, function, activity and/or stability of IGSF11 (VSIG3) or an IgC2 (or IgV) domain of IGSF11 or variant thereof.

In one further particular embodiment, the disease, disorder or condition is osteoporosis. IGSF11 regulates osteoclast differentiation through association with the scaffold protein PSD-95, and deletion of IGSF11 induces an increase in bone mass (Kim et al 2020a, Int J Mol Sci 21:2646; Kim et al 2020b, Bone Res 8:5). Accordingly, anti-IGSF11 ABPs could also be used in the treatment of osteoporosis.

According to the medical uses and methods of treatment disclosed herein, the subject is a mammal, and may include mice, rats, rabbits, monkeys and humans. In a preferred embodiment, the mammalian subject is a human patient.

In one embodiment, cells involved in the proliferative disorder are resistant to a cell-mediated immune response. For example, cells involved in the proliferative disorder (eg cells of a cancer or tumour) are resistant and/or refractory to blockade of an immune checkpoint molecule such as blockade using a ligand to an immune checkpoint molecule, in exemplary instances blockade of PD1/PDL1 and/or CTLA4 (analogous to Gao et al, 2017).

In particular, the treatment methods may be applied to a proliferative disorder that has been subjected to prior immunotherapy (such as therapy for blockade of an immune checkpoint molecule, eg blockade of PD1/PDL1 and/or CTLA4), in particular prior immunotherapy with a ligand to an immune checkpoint molecule. For example, in certain embodiments the IGSF11/domain binder and/or (eg antagonist) modulator, such as an ABP of the present invention, can be for use in the treatment of a proliferative disorder in a subject in need thereof, and the subject has been subjected to to prior immunotherapy, in particular prior administration of a ligand to an immune checkpoint molecule.

In other methods, the modulating (eg inhibiting) compound (eg an ABP, such as one of the present invention) may be used is in combination with a different anti-proliferative therapy, in particular a different anti-cancer therapy, in particular where the different anti-proliferative therapy is immunotherapy, in particular immunotherapy with a ligand to an immune checkpoint molecule. Accordingly, the composition can be for use in the treatment of a proliferative disorder in a subject in need thereof, where the subject is subjected to to co-treatment by immunotherapy, in particular co-therapy (eg combination treatment) with a ligand to an immune checkpoint molecule.

In such embodiments, the ligand is one that binds to an immune (inhibitory) checkpoint molecule. For example, such checkpoint molecule may be one selected from the group consisting of: A2AR, B7-H3, B7-H4, CTLA-4, IDO, KIR, LAG3, PD-1 (or one of its ligands PD-L1 and PD-L2), TIM-3 (or its ligand galectin-9), TIGIT and VISTA. In particular of such embodiments, the ligand binds to a checkpoint molecule selected from: CTLA-4, PD-1 and PD-L1. In other more particular embodiments, the ligand is an antibody selected from the group consisting of: ipilimumab, nivolumab, pembrolizumab, BGB-A317, atezolizumab, avelumab and durvaluma; in particular an antibody selected from the group consisting of: ipilimumab (YERVOY), nivolumab (OPDIVO), pembrolizumab (KEYTRUDA) and atezolizumab (TECENTRIQ).

When a method or use in therapy of the present invention (eg, one involving an ABP of the invention) is used in combination treatments together with any of such other procedures (eg, another agent or a cancer immunotherapy, such as a ligand that binds to an immune (inhibitory) checkpoint molecule), then such method or use being a combination treatment regimen may comprise embodiments where such exposures/administrations are concomitant. In alternative embodiments, such administrations may be sequential; in particular those embodiments where the IGSF11/domain binder and/or modulator (eg an ABP of the invention) is administered before such other procedure. For example, such IGSF11/domain binder and/or modulator may be sequentially administered within about 14 days of (eg before) the other procedure, such as within about 10 days, 7 days, 5 days, 2 days or 1 day of (eg before) the other procedure; and further including where the IGSF11/domain binder and/or modulator may be sequentially administered within about 48 hours, 24 hours, 12 hours, 8 hours, 6 hours, 4 hours, 2 hours, 1 hours, 30 mins, 15 mins or 5 mins of (eg before) the other procedure.

In certain embodiments, the medical uses or compositions are for use in enhancing an immune response in the subject, preferably for use in aiding a cell-mediated immune response in the subject such as the subject's T cell mediated immune response, for example for treating a proliferative disease such as a cancer disease.

In particular embodiments, the treatment can comprise a transfer of cells to the subject, preferably a transfer of immune cells to the subject, more preferably an adoptive T-cell transfer. For example, such cells can be autologous cells of the subject, for example autologous immune cells, such as T-cells, dendritic cells or Natural Killer (NK)-cells, of the subject.

In a preferred embodiment of the medical uses or compositions, the modulating compound (eg ABP of the invention) is an inhibitor or antagonist of expression, function, activity and/or stability of said IGSF11, or the IgC2 (or IgV) domain of IGSF11 or variant thereof, and wherein the inhibition of the expression, function, activity and/or stability of said IGSF11, domain or the variant thereof, enhances an immune response, preferably enhances a cell-mediated immune response in the subject such as a T-cell mediated immune response in the subject, for example for treating an infectious disease or a proliferative disease such as a cancer disease, in particular where the composition is an ABP of the invention.

In such embodiments, the immune response can be enhanced by an increase in T cell activity, proliferation and/or survival, in particular wherein the increase in T cell activity comprises an increase in production of one or more pro-inflammatory cytokines by such T cells (such as TILs). Preferably in such embodiments, the cytokine is one selected from the group consisting of: interleukin-1 (IL-1), IL-2, IL-12, IL-17, and IL-18, tumour necrosis factor (TNF) [alpha], interferon gamma (IFN-gamma), and granulocyte-macrophage colony stimulating factor, such as IL-2 (and/or IL-17 or IFN-gamma).

Such an increase in T cell activity, proliferation and/or survival, can be associated with the inhibition of the interaction between IGSF11/domain and an interacting protein (such as VSIR), in particular mediated by IGSF11-mediated VISTA signaling, or IGSF11-domain or -variant-mediated VISTA signaling.

Administration of the modulating (eg inhibiting) compound (eg an ABP of the invention) is, in certain embodiments, associated with the inhibition of the interaction between IGSF11/domain and VSIR, in particular mediated by IGSF11-mediated VISTA signaling, or IGSF11-domain or -variant-mediated VISTA signaling.

In other certain embodiments, administration of the modulating compound (eg an ABP of the invention), decreases or reduces the resistance of cells (such as tumour cells and/or cells that express IGSF11, or the IgC2 (or IgV) domain of IGSF11 or variant thereof), to an immune response, preferably wherein the compound enhances or increases the sensitivity of cells (such as tumour cells and/or cells that express IGSF11, or the IgC2 (or IgV) domain of IGSF11 or variant thereof), to an immune response.

In preferred embodiments, the medical uses are for the treatment of a proliferative disorder (such as a cancer described herein) in a mammalian subject in need thereof. In certain of such embodiments, the subject is a mouse, rat, guinea pig, rabbit, cat, dog, monkey, or preferably a human, for example a human patient.

Cells and Methods of Producing the ABPs/NACs of the Invention

As described above, in one aspect, herein provided is a cell, such as (recombinant) host cell or a hybridoma capable of expressing an ABP as described above. In an alternative aspect, herein provided is a cell, which comprises at least one NAC encoding an ABP or a component of an ABP as described above. Cells of the invention can be used in methods provided herein to produce the ABPs and/or NACs of the invention.

In certain embodiments, the cell is isolated or substantially pure, and/or is a recombinant cell and/or is a non-natural cell (i.e., it is not found in, or is a product of, nature), such as a hybridoma.

Accordingly, in another aspect the invention relates to a method of producing a recombinant cell line capable of expressing an ABP specific for IGSF11, or for an IGSF11 IgC2 (or IgV) domain, or a variant thereof, the method comprising the steps of:
providing a suitable host cell;
providing at least one genetic construct comprising coding sequence(s) encoding the ABP of the present invention;
introducing into said suitable host cell said genetic construct(s); and
optionally, expressing said genetic construct(s) by said suitable host cell under conditions that allow for the expression of the ABP.

In yet another aspect, herein provided is a method of producing an ABP as described above, for example comprising culturing one or more cells of the invention under conditions allowing the expression of said ABP.

Accordingly, in another aspect, the invention relates to a method of producing an ABP specific for IGSF11, or for an IGSF11 IgC2 (or IgV) domain, or a variant thereof, the method comprising the steps of:
providing a hybridoma or (host) cell capable of expressing an ABP according to the invention, for example a recombinant cell line comprising at least one genetic construct comprising coding sequence(s) encoding said compound or ABP; and
culturing said hybridoma or host cell under conditions that allow for the expression of the ABP.

For producing the recombinant ABPs of the invention, the DNA molecules encoding the proteins (e.g. for antibodies, light and/or heavy chains or fragments thereof) are inserted into an expression vector (or NAC) such that the sequences are operatively linked to transcriptional and translational control sequences. Alternatively, DNA molecules encoding the ABP can be chemically synthesized. Synthetic DNA molecules can be ligated to other appropriate nucleotide sequences, including, e.g., constant region coding sequences, and expression control sequences, to produce conventional gene expression constructs encoding the desired ABP. For manufacturing the ABPs of the invention, the skilled artisan may choose from a great variety of expression systems well known in the art, e.g. those reviewed by Kipriyanow and Le Gall, 2004. Expression vectors include, but are not limited to, plasmids, retroviruses, cosmids, EBV-derived episomes, and the like. The term “expression vector” or “NAC” comprises any vector suitable for the expression of a foreign DNA. Examples of such expression vectors are viral vectors, such as adenovirus, vaccinia virus, baculovirus and adeno-associated virus vectors. In this connection, the expression “virus vector” is understood to mean both a DNA and a viral particle. Examples of phage or cosmid vectors include pWE15, M13, AEMBL3, AEMBL4, AFIXII, ADASHII, AZAPII, AgT10, Agtll, Charon4A and Charon21A. Examples of plasmid vectors include pBR, pUC, pBluescriptII, pGEM, pTZ and pET groups. Various shuttle vectors may be used, e.g., vectors which may autonomously replicate in a plurality of host microorganisms such as E. coli and Pseudomonas sp. In addition, artificial chromosome vectors are considered as expression vectors. The expression vector and expression control sequences are selected to be compatible with the cell, such as a host cell. Examples of mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRepS, D H26S, D HBB, pNMT1, pNMT41, pNMT81, which are available from Invitrogen™, pCI which is available from Promega, pMbac, pPbac, pBK-RSV and pBK-CMV which are available from Agilent Technologies, pTRES which is available from Clontech, and their derivatives.

For manufacturing antibodies, the antibody light chain gene and the antibody heavy chain gene can be inserted into separate vectors. In certain embodiments, both DNA sequences are inserted into the same expression vector. Convenient vectors are those that encode a functionally complete human CH or CL immunoglobulin sequence, with appropriate restriction sites engineered so that any VH or VL sequence can be easily inserted and expressed, as described above, wherein the CH1 and/or upper hinge region comprises at least one amino acid modification of the invention. The constant chain is usually kappa or lambda for the antibody light chain. The recombinant expression vector may also encode a signal peptide that facilitates secretion of the antibody chain from a (host) cell. The DNA encoding the antibody chain may be cloned into the vector such that the signal peptide is linked in-frame to the amino terminus of the mature antibody chain DNA. The signal peptide may be an immunoglobulin signal peptide or a heterologous peptide from a non-immunoglobulin protein. Alternatively, the DNA sequence encoding the antibody chain may already contain a signal peptide sequence.

In addition to the DNA sequences encoding the ABP (antibody) chains, the recombinant expression vectors carry regulatory sequences including promoters, enhancers, termination and polyadenylation signals and other expression control elements that control the expression of the antibody chains in a (host) cell. Examples for promoter sequences (exemplified for expression in mammalian cells) are promoters and/or enhancers derived from CMV (such as the CMV Simian Virus 40 (SV40) promoter/enhancer), adenovirus, (e.g., the adenovirus major late promoter (AdMLP)), polyoma and strong mammalian promoters such as native immunoglobulin and actin promoters. Examples for polyadenylation signals are BGH polyA, SV40 late or early polyA; alternatively, 3UTRs of immunoglobulin genes etc. can be used.

The recombinant expression vectors may also carry sequences that regulate replication of the vector in (host) cells (e.g. origins of replication) and selectable marker genes. Nucleic acid molecules encoding the heavy chain or an antigen-binding portion thereof and/or the light chain or an antigen-binding portion thereof of an antibody of the present invention, and vectors comprising these DNA molecules can be introduced into (host) cells, e.g. bacterial cells or higher eukaryotic cells, e.g. mammalian cells, according to transfection methods well known in the art, including liposome-mediated transfection, polycation-mediated transfection, protoplast fusion, microinjections, calcium phosphate precipitation, electroporation or transfer by viral vectors.

For antibodies or fragments thereof, it is within ordinary skill in the art to express the heavy chain and the light chain from a single expression vector or from two separate expression vectors. Preferably, the DNA molecules encoding the heavy chain and the light chain are present on two vectors which are co-transfected into the (host) cell, preferably a mammalian cell

Mammalian cell lines available as hosts for expression are well known in the art and include, inter alia, Chinese hamster ovary (CHO, CHO-DG44, BI-HEX-CHO) cells, NSO, SP2/0 cells, HeLa cells, HEK293 cells, baby hamster kidney (BHK) cells, monkey kidney cells (COS), human carcinoma cells (e.g., Hep G2), A549 cells, 3T3 cells or the derivatives/progenies of any such cell line. Other mammalian cells, including but not limited to human, mice, rat, monkey and rodent cells lines, or other eukaryotic cells, including but not limited to yeast, insect and plant cells, or prokaryotic cells such as bacteria may be used. The antibody molecules of the invention are produced by culturing the host cells for a period of time sufficient to allow for expression of the antibody molecule in the host cells.

According to some embodiments of the method of producing an ABP, following expression, the intact antibody (or the antigen-binding fragment of the antibody) can be harvested and isolated using purification techniques well known in the art, e.g., Protein A, Protein G, affinity tags such as glutathione-S-transferase (GST) and histidine tags.

ABPs are preferably recovered from the culture medium as a secreted polypeptide or can be recovered from host cell lysates if for example expressed without a secretory signal. It is necessary to purify the ABP molecules using standard protein purification methods used for recombinant proteins and host cell proteins in a way that substantially homogenous preparations of the ABP are obtained. By way of example, state-of-the art purification methods useful for obtaining the ABP molecule of the invention include, as a first step, removal of cells and/or particulate cell debris from the culture medium or lysate. The ABP is then purified from contaminant soluble proteins, polypeptides and nucleic acids, for example, by fractionation on immunoaffinity or ion-exchange columns, ethanol precipitation, reverse phase HPLC, Sephadex chromatography, chromatography on silica or on a cation exchange resin. Preferably, ABPs are purified by standard protein A chromatography, e.g., using protein A spin columns (GE Healthcare). Protein purity may be verified by reducing SDS PAGE. ABP concentrations may be determined by measuring absorbance at 280 nm and utilizing the protein specific extinction coefficient. As a final step in the process for obtaining an ABP molecule preparation, the purified ABP molecule may be dried, e.g. lyophilized, for therapeutic applications.

Accordingly, certain embodiments of such aspects, the method comprises a further step of isolation and/or purification of the ABP.

In another aspect, herein provided is a method of manufacturing a pharmaceutical composition comprising an ABP as described above, comprising formulating the ABP isolated by the methods described above into a pharmaceutically acceptable form.

In an alternative aspect, herein provided is a method of manufacturing a pharmaceutical composition comprising an NAC as described above, comprising formulating the NAC prepared by the methods described above into a pharmaceutically acceptable form.

According to some embodiments, the methods of manufacturing a pharmaceutical composition comprise a further step of combining said ABP and/or NAC with a pharmaceutically acceptable excipient or carrier.

In some embodiments of the method of manufacturing a pharmaceutical composition comprising an ABP, the ABP typically will be labelled with a detectable labelling group before being formulated into a pharmaceutically acceptable form. Various methods for labelling proteins are known in the art and may be used. Suitable labelling groups include, but are not limited to, the following: radioisotopes or radionuclides (e.g., 3H, 14C, 15N, 35S, 90Y, 99Tc, 111In, 125I, 131I), fluorescent groups (e.g., FITC, rhodamine, lanthanide phosphors), enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labelling group is coupled to the ABP via spacer arms of various lengths to reduce potential steric hindrance.

Accordingly, in certain embodiments of such aspects, the ABP is a modified antibody and the method comprises a further step of addition of a functional moiety selected from a detectable labelling group or a cytotoxic moiety.

Detection/Diagnostic/Monitoring Aspects

IGSF11 or an IgC2 domain of (or an IgV domain of) IGSF11, or a variant thereof, can be used for diagnostic purposes to detect, diagnose, or monitor diseases, disorders and/or conditions associated with the undesired presence of IGSF11/domain-positive cells or cells positive for a variant thereof and/or associated with cellular resistance against a cell-mediated immune response; and in particular aberrant and/or localised expression/activity of IGSF11/domain (in particular phosphorylated IGSF11/domain) can be so used. The disease, disorder and/or conditions so detected, diagnosed, or monitored, can be one of those described elsewhere herein. In preferred embodiments of the detection and diagnosis methods of the invention, the diseases, disorders or conditions is a proliferative disorder, such as cancer or tumour (eg a solid tumour), including one or more of those described elsewhere herein; more preferably one or more of lung cancer, breast cancer, colorectal cancer, pancreatic cancer, gastric cancer, hepatocellular carcinoma, ovarian cancer, melanoma, myeloma, kidney cancer, head and neck cancer, Hodgkin lymphoma, bladder cancer or prostate cancer, in particular one selected from the list consisting of: melanoma, lung cancer (such as non-small cell lung cancer), bladder cancer (such as urothelial carcinoma), kidney cancer (such as renal cell carcinoma), head and neck cancer (such as squamous cell cancer of the head and neck) and Hodgkin lymphoma. Preferably, the proliferative disease is melanoma, or lung cancer (such as non-small cell lung cancer).

Accordingly, in a sixth aspect, the invention related to a method for determining (eg an in vitro method) whether a subject has, or is at risk of, developing a phenotype (eg a disease, disorder or condition) that is associated with the undesired presence of IGSF11/domain-positive cells or cells positive for a variant thereof and/or that is associated with cellular resistance against a cell-mediated immune response and/or that is associated with (eg aberrant) expression or activity of IGSF11 or of an IgC2 (or IgV) domain of IGSF11 (or a variant thereof), the method comprising the step of:
detecting (for example, detecting in vitro), eg protein and/or mRNA of IGSF11 or of an IgC2 (or IgV) domain of IGSF11 (or a variant thereof), in particular the presence (or an amount) of or expression and/or activity of IGSF11 or of an IgC2 (or IgV) domain of IGSF11 (or the variant), in a biological sample from said subject,
wherein the detection of IGSF11 or of an IgC2 (or IgV) domain of IGSF11 (or the variant thereof) in the sample indicates such phenotype (eg such disease, disorder or condition), or a risk of developing such phenotype (eg such disease, disorder or condition), in the subject.

In certain embodiments of such aspect, the detection of the IGSF11 or domain (or the variant) may comprise determining the presence or an amount of the IGSF11 (or the variant), or activity thereof, in the sample, in particular the IGSF11 or domain (or the variant) associated with or of tumour cells (or immune cells present at the site of the tumour) of the subject. In other (alternative or further) embodiments, the detection may comprise determining the presence or an amount of one or more of other applicable biomarkers such as VSIR protein and/or mRNA.

In a preferred embodiment, protein IGSF11 or an IgC2 (or IgV) domain of IGSF11 protein (or a variant thereof) is detected with a ABP of the invention, and in an alternative embodiment mRNA IGSF11 an IgC2 (or IgV) domain of IGSF11 mRNA (or a variant thereof) is detected.

In a preferred embodiment, protein IGSF11 or an IgC2 (or IgV) domain of IGSF11 protein (or a variant thereof) is detected in in a biological sample being plasma (or serum) from said subject. For example, the IgC2 (or IgV) domain of IGSF11 protein may be detected in plasma of a cancer patient where the tumour has shed ECD of IGSF11 into the bloodstream.

In a related aspect, the invention relates to a method for determining the presence or an amount of IGSF11 or of an IgC2 (or IgV) domain of IGSF11 (or a variant thereof) in a biological sample from a subject, the method comprising the steps of:
contacting said sample with an ABP capable of binding to IGSF11 or to an IgC2 (or IgV) domain of IGSF11 (or the variant); and
detecting binding between the IGSF11 or the IgC2 (or IgV) domain of IGSF11 (or the variant) in the biological sample and the ABP.

In a preferred embodiment, protein IGSF11 or an IgC2 (or IgV) domain of IGSF11 protein (or a variant thereof) is detected with a ABP of the invention.

In certain embodiments, a biological sample will (preferably) comprise cells or tissue of the subject, or an extract of such cells or tissue, in particular where such cells are those involved with the proliferative disorder (eg tumour cells such as cells of a solid tumour, or immune cells present at the site of the tumour). The tumour or cell thereof, may be one or, or derived from, one of the tumours described elsewhere herein.

In particular embodiments of such aspect, the method will also comprise a step of:
providing (such as by obtaining) the biological sample from the subject, in particular where such step is conducted prior to the detection step.

In particular embodiments, such detection and/or determination methods can be practiced as a method of diagnosis, such as a method of diagnosis whether a mammalian subject (such as a human subject or patient) has a disease, disorder or condition (such as one described above), in particular a proliferative disorder such as a cancer or tumour (or has a risk of developing such a disease, disorder or condition) that is associated with the undesired presence of IGSF11/domain-positive cells or cells positive for an an IgC2 (or IgV) domain of IGSF11, or for a variant thereof, and/or that is associated with cellular resistance against a cell-mediated immune response and/or that is associated with (eg aberrant) expression or activity of IGSF11 or of an IgC2 (or IgV) domain of IGSF11 (or a variant thereof); in particular a (solid) tumour, such as one having cellular resistance against a cell-mediated immune response.

In certain embodiments of these detection, determination and/or diagnostic methods, the cellular resistance against a cell-mediated immune response is cellular resistance against a T cell-mediated immune response.

In certain embodiments, the biological sample is one obtained from a mammalian subject like a human patient. The term “biological sample” is used in its broadest sense and can refer to a bodily sample obtained from the subject (eg, a human patient). For example, the biological sample can include a clinical sample, i.e., a sample derived from a subject. Such samples can include, but are not limited to: peripheral bodily fluids, which may or may not contain cells, e.g., blood, urine, plasma, mucous, bile pancreatic juice, supernatant fluid, and serum; tissue or fine needle biopsy samples; tumour biopsy samples or sections (or cells thereof), and archival samples with known diagnosis, treatment and/or outcome history. Biological samples may also include sections of tissues, such as frozen sections taken for histological purposes. The term “biological sample” can also encompass any material derived by processing the sample. Derived materials can include, but are not limited to, cells (or their progeny) isolated from the biological sample, nucleic acids and/or proteins extracted from the sample. Processing of the biological sample may involve one or more of, filtration, distillation, extraction, amplification, concentration, fixation, inactivation of interfering components, addition of reagents, and the like. In one embodiment the biological sample is a plasma (or serum) sample (previously taken) from the subject.

In some embodiments, these detection, determination and/or diagnostic methods may be a computer-implemented method, or one that is assisted or supported by a computer. In some embodiments, information reflecting the presence or an amount of the IGSF11 or the domain (or variant thereof) to be determined (or activity thereof) in a sample is obtained by at least one processor, and/or information reflecting the presence or an amount of the IGSF11, domain or variant (or activity thereof) in a sample is provided in user readable format by another processor. The one or more processors may be coupled to random access memory operating under control of or in conjunction with a computer operating system. The processors may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. Processors may communicate with data storage devices, such as a database stored on a hard drive or drive array, to access or store program instructions other data. Processors may further communicate via a network interface, which in turn may communicate via the one or more networks, such as the Internet or other public or private networks, such that a query or other request may be received from a client, or other device or service. In some embodiments, the computer-implemented method of detecting the presence or an amount of the IGSF11, domain or variant (or activity thereof) in a sample is provided as a kit.

Such detection, determination and/or diagnosis methods can be conducted as an in-vitro method, and can be, for example, practiced using the kit of the present invention (or components thereof).

In some embodiments of these detection, determination and/or diagnosis methods, the biological sample is a tissue sample from the subject, such as a sample of a tumour or a cancer from the subject. Such a sample may contain tumour cells and/or blood cells (eg monocytes and T cells). As described above, such tissue sample may be a biopsy sample of the tumour or a cancer such as a needle biopsy samples, or a tumour biopsy sections or an archival sample thereof. Such a tissue sample may comprise living, dead or fixed cells, such as from the tumour or a cancer, and such cells may be suspected of expressing (e.g. aberrantly or localised) the applicable biomarker to be determined.

In other embodiments of these detection, determination and/or diagnosis methods, the biological sample is a blood sample from the subject, such as a sample of immune cells present in blood (eg monocytes and T cells).

In some embodiments, determination and/or diagnosis method of the invention can comprise, such as in a further step, comparing the detected amount (or activity of) of (eg protein or mRNA of) the applicable biomarker (ie IGSF11/domain or a variant thereof) with a standard or cut-off value; wherein a detected amount greater than the standard or cut-off value indicates a phenotype (or a risk of developing a phenotype) that is associated with the undesired presence of IGSF11/domain-positive cells (or cells positive for a variant of IGSF11) and/or that is associated with cellular resistance against the cell-mediated immune response in the subject and/or is associated with (eg aberrant) expression or activity of IGSF11 or of an IgC2 (or IgV) domain of IGSF11 (or the variant) in the subject. Such a standard or cut-off value may be determined from the use of a control assay, or may be pre-determined from one or more values obtained from a study or a plurality of samples having known phenotypes. For example, a cut-off value for a diagnostic test may be determined by the analysis of samples taken from patients in the context of a controlled clinical study, and determination of a cut-off depending on the desired (or obtained) sensitivity and/or specificity of the test.

Examples of methods useful in the detection of (such as the presence or absence of, or an amount of) the applicable biomarker (ie the IGSF11, domain or variant thereof) include immunoassays, such as the enzyme linked immunosorbent assay (ELISA) and the radioimmunoassay (RIA), which employ ABP (eg of the present invention) such as an antibody or an antigen-binding fragment thereof, that specifically binds to such applicable biomarker.

For such methods, a monoclonal antibody or a polyclonal antibody may be employed. Examples of monoclonal antibodies are described elsewhere herein. The term “polyclonal antibody” as used herein refers to a mixture of antibodies which are genetically different since produced by plasma cells derived from multiple somatic recombination and clonal selection events and which, typically, recognise a different epitope of the same antigen.

Alternatively, the presence of the applicable biomarker (ie IGSF11/domain or variant thereof) may be detected by detection of the presence of mRNA that encodes such applicable biomarker, or fragments of such mRNA. Methods to detect the presence of such mRNA (or fragments) can include, PCR (such as quantitative RT-PCR), hybridisation (such as to Illumina chips), nucleic-acid sequencing etc. Such methods may involve or comprise steps using one or more nucleic acids as described herein, such as PCR primers or PCR probes, or hybridisation probes, that bind (eg specifically) to such mRNA.

For such detection, determination or diagnostic applications, the ABP or nucleic acid, typically, will be labelled with a detectable labelling group. In general, labelling groups fall into a variety of classes, depending on the assay in which they are to be detected: a) isotopic labels, which may be radioactive or heavy isotopes; b) magnetic labels (e.g., magnetic particles); c) redox active moieties; d) optical dyes; enzymatic groups (e.g. horseradish peroxidase, beta-galactosidase, luciferase, alkaline phosphatase); e) biotinylated groups; and f) predetermined polypeptide epitopes recognised by a secondary reporter (e.g., leucine zipper pair sequences, binding sites for secondary antibodies, metal binding domains, epitope tags, etc.). Suitable labelling groups include, but are not limited to, the following: radioisotopes or radionuclides (e.g., ³H, ⁴C, ¹⁵N, ³⁵S, ⁹⁰Y, ⁹⁹Tc, ¹¹¹In, ¹²⁵I, 131I), fluorescent groups (e.g., FITC, rhodamine, lanthanide phosphors), enzymatic groups (e.g., horseradish peroxidase, beta-galactosidase, luciferase, alkaline phosphatase), chemiluminescent groups, biotinyl groups, or predetermined polypeptide epitopes recognised by a secondary reporter (eg, leucine zipper pair sequences, binding sites for secondary antibodies, metal binding domains, epitope tags). In some embodiments, the labelling group is coupled to the ABP or nucleic acid via spacer arms of various lengths to reduce potential steric hindrance. Various methods for labelling proteins are known in the art and may be used. For example, the ABP or nucleic acid may be labelled with a secondary reporter (e.g., leucine zipper pair sequences, binding sites for secondary antibodies, metal binding domains, epitope tags, etc.).

Accordingly, in particular embodiments of the detection/diagnostic methods (or the kits therefor), the means (eg ABP or nucleic acid) for the detection (eg detector) of protein or mRNA of the applicable biomarker (eg IGSF11), is labelled, for example is coupled to a detectable label. The term “label” or “labelling group” refers to any detectable label, including those described herein.

In certain embodiments, the detection/diagnostic methods of the invention involve an immunohistochemistry (IHC) assay or an immunocytochemistry (IC) assay. The terms “IHC” and “ICC” are art recognised, and include the meanings of techniques employed to localise antigen expression that are dependent on specific epitope-antibody interactions. IHC typically refers to the use of tissue sections, whereas ICC typically describes the use of cultured cells or cell suspensions. In both methods, positive staining is typically visualised using a molecular label (eg, one which may be fluorescent or chromogenic). Briefly, samples are typically fixed to preserve cellular integrity, and then subjected to incubation with blocking reagents to prevent non-specific binding of the antibodies. Samples are subsequently typically incubated with primary (and sometimes secondary) antibodies, and the signal is visualised for microscopic analysis.

Accordingly, such embodiments of the detection/diagnostic methods of the invention may include a step of preparing a subject IHC or ICC preparation tissue or cells (such as those present in the biological samples obtained from a subject); and preferably wherein the detection of binding of an ABP to the applicable biomarker (ie IGSF11/domain or a variant thereof) expressed by the tissues of cells said IHC or ICC preparation indicates: (i) a phenotype such as a disease, disorder or condition (or a risk of developing such a phenotype) that is associated with the undesired presence of IGSF11/domain-positive cells (or cells positive for an IgC2 (or IgV) domain of IGSF11, or a variant thereof) and/or associated with cellular resistance against the cell-mediated immune response in the subject; and/or (ii) said subject has or has a risk of developing disease, condition or disorder that is associated with (eg aberrant) expression or activity of the IGSF11, domain or variant.

In such IHC/ICC methods is used an ABP that binds to (preferably specifically to) the applicable biomarker (ie IGSF11/domain or a variant thereof) and that does not bind (eg does not detectably bind) to a validation IHC or ICC preparation of mammalian tissues or cells other than to (detectably) bind to the applicable biomarker (ie IGSF11/domain or a variant thereof) that is expressed by the tissue cells or of said validation IHC or ICC preparation.

In certain of such embodiment, said validation and/or subject IHC or ICC preparation is one selected from the list consisting of: a frozen section, a paraffin section, and a resin section, in each case of the tissues and/or cells; and/or wherein the tissues and/or cell comprised in either (or both) said IHC or ICC preparations are fixed. The tissues and/or cells or such IHC or ICC preparation(s) may be fixed by an alcohol, an aldehyde, a mercurial agent, an oxidising agent or a picrate.

In one preferred of such embodiments, said validation and/or subject IHC or ICC preparation is a formalin-fixed paraffin embedded (FFPE) section of said tissues and/or cells; and/or wherein said validation and/or subject IHC or ICC preparation is subjected to antigen retrieval (AR). Such AR may comprise protease-induced epitope retrieval (PIER) or heat-induced epitope retrieval (HIER).

The ABP used in such methods is, preferably, validated. For example, the ABP is validated to (detectably) bind to the applicable biomarker (ie IGSF11/domain or a variant thereof) expressed by the cells and/or tissues of said validation IHC or ICC preparation, but does not (detectably) bind to a control IHC or ICC preparation of control cells and/or tissues that do not express such applicable biomarker. Preferably, said control cells are gene knock-down or gene knock-out cells and/or tissues for the applicable biomarker (ie IGSF11/domain or a variant thereof); more preferably, wherein said gene knock-down or gene knock-out cells and/or tissues are siRNA or shRNA gene knock-down or gene knock-out for such applicable biomarker. Such control cells may comprise control cells and/or tissues that do not express such applicable biomarker (ie IGSF11/domain or a variant thereof) comprise cells of said cell line that have been transfected with a IGSF11 siRNA selected from those of Table A (or transfected with a shRNA as described above); and/or said validation IHC or ICC preparation comprises cells transduced with shIGSF11 lentiviral vectors. Alternatively, the selectivity of such ABP can be determined by binding to recombinant, cell surface expressed IGSF11 or an IgC2 (or IgV) domain of IGSF11, or a variant thereof, versus no binding to the same cell line expressing an irrelevant recombinant antigen.

In such IHC/ICC methods, the ABP is used with said validation and/or subject IHC or ICC preparation at a working concentration of less than about 50 ug/mL, 25 ug/mL, 20 ug/mL, 15 ug/mL, 10 ug/mL, 7.5 ug/mL, 5 ug/mL, 2.5 ug/mL, 1 ug/mL, 0.5 ug/mL, 0.2 ug/ml or 0.1 ug/ml, in particular less than about 5 ug/mL, and more particularly at less than 2.5 ug/mL; preferably, at a concentration that is about 2-fold, 5-fold, 10-fold, 20-fold or 50-fold higher than said working concentration, said ABP does not (detectable) bind to said validation immunohistochemistry (IHC) preparation of mammalian cells or tissues other than to (detectably) bind to the applicable biomarker (ie IGSF11/domain or a variant thereof) expressed by the mammalian cells or tissue of said IHC preparation, in particular at a concentration that is about 2-fold higher than said working concentration, and more particularly at a concentration that is about 5-fold higher than said working concentration

In the detection/diagnostic methods of the invention, the ABP used may be a polyclonal antibody; and preferably may be a rabbit antibody.

In another aspect, the invention relates to a method for determining whether a subject has, or has a risk of developing, a disease, disorder or condition that is associated with the undesired presence of IGSF11-positive cells or cells positive for a variant of IGSF11 and/or that is associated with cellular resistance against a cell-mediated immune response and/or that is associated with (eg aberrant) expression or activity of IGSF11 (or a variant thereof), the method comprising the steps of:
contacting cells of the subject involved with the disease, disorder or condition with an (eg IGSF11/domain inhibitory) ABP of the invention, or a (eg IGSF11/domain inhibitory) product or another (eg IGSF11/domain inhibitory) modulating compound of the invention, in the presence of a cell-mediated immune response, preferably wherein the cell-mediated immune response comprises immune cells selected from the group consisting of: lymphocytes, T-cells, CTLs and TILs; and
determining the cell-mediated immune response against such cells of the subject, wherein an enhancement of the cell-mediated immune response against such cells of the subject indicates that the subject has or has a risk of developing such disease, disorder or condition, such as a proliferative disorder or infectious disease (preferably, a proliferative disorder such as a cancer).

In an alternative aspect, the invention relates to a method for determining whether a subject has, or has a risk of developing, a disease, disorder or condition that is associated with the undesired presence of IGSF11-positive cells or cells positive for a variant of IGSF11 and/or that is associated with (eg aberrant) expression or activity of IGSF11 (or a variant thereof), the method comprising the steps of:
contacting cells of the subject involved with the disease, disorder or condition with an (eg IGSF11/domain activating) ABP of the invention, or a (eg IGSF11/domain activating) product or another (eg IGSF11/domain activating) modulating compound of the invention, in the presence of a cell-mediated immune response, preferably wherein the cell-mediated immune response comprises immune cells selected from the group consisting of: lymphocytes, T-cells, CTLs and TILs; and
determining the cell-mediated immune response against such cells of the subject, wherein a reduction of the cell-mediated immune response against such cells of the subject indicates that the subject has or has a risk of developing such disease, disorder or condition, such as autoimmunity, allergy or inflammatory conditions.

In a related aspect, the invention relates to a method for determining the resistance of a cell involved with a proliferative disease (eg a cancer or tumour) to a cell-mediated immune response, the method comprising the steps or:
contacting such cells with an (eg IGSF11/domain inhibitory) ABP of the invention, or a (eg IGSF11/domain inhibitory) product or another (eg IGSF11/domain inhibitory) modulating compound of the invention, in the presence of a cell-mediated immune response, such as immune cells selected from the group consisting of: lymphocytes, T-cells, CTLs and TILs; and
determining the cell-mediated immune response against such cells;
wherein an enhancement of the cell-mediated immune response against such cells (in the presence of the ABP of the invention or other product or modulating compound of the invention) indicates that such cells have a resistance to a cell-mediated immune response.

In certain embodiments, the cells involved with a proliferative disorder are provided as a biological sample obtained from a subject (such as a human subject or patient) that has (or has a risk of developing) a disease, disorder or condition that is associated with the undesired presence of IGSF11/domain-positive cells (or cells positive for an IgC2 (or IgV) domain of IGSF11, or a variant thereof) and/or associated with cellular resistance against a cell-mediated immune response and/or that is associated with (eg aberrant) expression or activity of IGSF11 or of an IgC2 (or IgV) domain of IGSF11, or a variant thereof.

In certain embodiments, the cells of the subject contacted with the cell-mediated immune response are provided (such as by obtaining) a biological sample from the subject, wherein the sample comprises cells of the subject (such as cells of a tumour or cancer of the subject). Particular embodiments of such method also comprise a step of providing (such as by obtaining) a biological sample from the subject, in particular where such step is conducted prior to the contacting step.

In a related aspect, the detection, a determination and/or diagnostic method may be used as a method for monitoring (or prognosing) the success (or likelihood of success or risk or remission) of treatment of a subject being treated, or intended to be treated, with a treatment method of the invention. For example: (1) if the sample from the subject is determined to contain the presence of (or an indicative amount of) IGSF11 (or an IgC2 (or IgV) domain of IGSF11, or a variant thereof), then this indicates that (a future) treatment with a method of the invention (eg administration of an ABP of the invention) may be successful, or more likely to be successful, for such subject; and/or (2) if, during the course of such treatment (eg administration of an ABP of the invention), a reduction in (such as less than an indicative amount of), the absence of, or the functional inhibition of (eg, by monitoring the phosphorylation status), IGSF11 (or an IgC2 (or IgV) domain of IGSF11, or a variant thereof), or expression (or activity) thereof, is determined in the sample from the subject, then this indicates that such treatment with a method of the invention (eg administration of an ABP of the invention) is or was successful, or is more likely to be successful if continued, for such subject.

The person of ordinary skill will now readily recognise how the detection, determination and/or diagnostic methods of the present invention (and any embodiments thereof) may be practiced or modified so as to use them as part of the monitoring or prognostic methods of the invention.

In another aspect, the invention relates to a method of diagnosing and treating a disease, disorder or condition characterised by the undesired presence of IGSF11-positive cells (or cells positive for a variant of IGSF11) and/or by cellular resistance against a cell-mediated immune response and/or by (eg aberrant) expression or activity of IGSF11 (or a variant thereof), (such as a proliferative disorder, eg a tumour or cancer) in a subject, such as a human patient, comprising:
conducting a detection, determination and/or diagnostic method of the invention (such as one described above), thereby diagnosing if the subject is suffering from such a disease, disorder or condition; and
administering an effective amount of an ABP of the invention (or another modulating compound of the invention), and/or a pharmaceutical composition of the invention, to the so diagnosed subject, in particular practicing a treatment method of the invention on the subject.

In yet another aspect, the invention relates to an ABP binding to (preferably specifically to) protein of the applicable biomarker (ie IGSF11 or an IgC2 (or IgV) domain of IGSF11, or a variant thereof), or a nucleic acid that can bind to (such as specifically to) mRNA of such applicable biomarker, for use in diagnosis, such as in the detection of (or determination of the risk of developing) a disease, disorder or condition in a mammalian subject, such as a human patient, in particular of a disease, disorder or condition that is associated with the undesired presence of IGSF11-positive cells (or cells positive for a variant of IGSF11) and/or that is associated with cellular resistance against a cell-mediated immune response (such as a proliferative disorder, eg a tumour or cancer), and/or that is associated with (eg aberrant) expression or activity of IGSF11 or a variant thereof.

Accordingly, one embodiment of such aspect provides a use of an ABP that is capable of binding to or binds to (eg specifically to) IGSF11 or an IgC2 (or IgV) domain of IGSF11, or a variant thereof (in particular, an ABP of the invention) for/in (eg, in-vitro) diagnosis. In particular is provided an ABP (such as a monoclonal antibody) that binds to (eg specifically to) IGSF11 or an IgC2 (or IgV) domain of IGSF11, or a variant thereof, for use in the diagnosis of a disease, disorder or condition that is associated with the undesired presence of IGSF11-positive cells (or cells positive for an IgC2 (or IgV) domain of IGSF1, or positive for a variant thereof) and/or that is associated with cellular resistance against a cell-mediated immune response (such as a cancer), and/or that is associated with (eg aberrant) expression or activity of IGSF11 or of an IgC2 (or IgV) domain of IGSF11, or a variant thereof.

The ABP or nucleic acid for use for such detection may be any as described elsewhere herein.

Detection/Diagnostic/Monitoring Kits:

In a seventh aspect, herein provided is a kit, such as one for performing the diagnostic methods or the determination methods or the detection methods (or the monitoring or prognostic methods) of the invention, eg, for determining the presence, absence, amount, function, activity and/or expression of the applicable biomarker (ie IGSF11 or an IgC2 (or IgV) domain of IGSF11, or a variant thereof) in a sample (eg a biological sample), such as on cells in a sample. The kit comprises an ABP and/or a nucleic acid as described above and, optionally one or more additional components.

In certain embodiments of the kit, an additional component may comprise instructions describing how to use the ABP or a nucleic acid or kit, for detecting the presence of the applicable biomarker in the sample, such as by detecting binding between the ABP and protein such applicable biomarker, and/or detecting binding between the nucleic acid and mRNA of such applicable biomarker. Such instructions may consist of a printed manual or computer readable memory comprising such instructions, or may comprise instructions as to identify, obtain and/or use one or more other components to be used together with the kit.

In other certain embodiments of the kit, the additional component may comprise one or more other claim, component, reagent or other means useful for the use of the kit or practice of a detection method of the invention, including any such claim, component, reagent or means disclosed herein useful for such practice. For example, the kit may further comprise reaction and/or binding buffers, labels, enzymatic substrates, secondary antibodies and control samples, materials or moieties etc.

In a particular such embodiment, the additional component may comprise means of detecting the presence of protein of the applicable biomarker (ie IGSF11 or an IgC2 (or IgV) domain of IGSF11, or a variant thereof), such as detecting binding between the ABP and such protein.

Various means for indicating (eg indictors) the binding of an ABP can be used. For example, fluorophores, other molecular probes, or enzymes can be linked to the ABP and the presence of the ABP can be observed in a variety of ways. A method for screening for diseases, disorders or conditions can involve the use of the kit, or simply the use of one of the disclosed ABPs and the determination of the extent to which ABP binds to the protein of the applicable biomarker (ie IGSF11 or an IgC2 (or IgV) domain of IGSF11, or a variant thereof), in a sample. As will be appreciated by one of skill in the art, high or elevated levels of protein of such applicable biomarker, will result in larger amounts of the ABP binding thereto in the sample. Thus, degree of ABP binding can be used to determine how much of such applicable biomarker is in a sample. Subjects or samples with an amount of such applicable biomarker that is greater than a predetermined amount (eg, an amount or range that a person without a disorder related to the applicable biomarker (ie IGSF11 or an IgC2 (or IgV) domain of IGSF11, or a variant thereof) can be characterised as having a disease, disorder or condition mediated by IGSF11 or by an IgC2 (or IgV) domain of IGSF11, or a variant thereof (such as one mediated by the (eg aberrant) expression, function, activity and/or stability of IGSF11 or of an IgC2 (or IgV) domain of IGSF11, or the variant), in particular of IGSF11 or of an IgC2 (or IgV) domain of IGSF11, or the variant in tumour cells.

In some embodiments, the kit further comprises one or more of the following: standards of protein or mRNA of the applicable biomarker (ie IGSF11 or an IgC2 (or IgV) domain of IGSF11, or a variant thereof), positive and/or negative controls for ABP or nucleic acid binding, a vessel for collecting a sample, materials for detecting binding of the ABP or nucleic acid to protein or mRNA (as applicable) of such applicable biomarker in said sample, and reagent(s) for performing said detection.

In another aspect herein provided is the use of a kit as described above for performing the (eg in vitro) diagnostic or detection methods of the invention; and, in a related other aspect the invention related to a kit as described above for use in a (eg in vitro) determination/diagnostic method of the present invention.

As described above, kits of the invention may be accompanied by instructions, including those to use them for determining the amount, activity and/or expression of the applicable biomarker (ie IGSF11 or an IgC2 (or IgV) domain of IGSF11, or a variant thereof), such as in tumour cells in a sample.

Screening Aspects of the Invention:

In an eighth aspect of the invention is provided, a method for identifying (and/or characterising) a compound, such as a compound suitable for use in medicine (such as for the treatment of a disease, disorder or condition, eg a proliferative disorder) that is associated with the undesired presence of IGSF11-positive cells or cells positive for a variant of IGSF11 and/or that is characterised by cellular resistance against a cell-mediated immune response and/or one that is characterised by (aberrant) expression or activity of IGSF11 or a variant thereof, the method comprising the steps of:
bringing into contact a first cell and the candidate compound, wherein the first cell expresses IGSF11 or an IgC2 (or IgV) domain of IGSF11, or a variant thereof (eg, a protein or mRNA of the IGSF11, domain or variant); and
determining (i) the expression, activity (eg kinase activity), function and/or stability of the (eg protein or mRNA of) the IGSF11, domain or variant, in the first cell (eg, and ABP may induce internalisation of IGSF11 protein, or such domain of IGSF11 protein, from the surface of the first cell); and/or (ii) the cell-mediated immune response (eg the cytotoxicity or cytokine production) against the first cell,
wherein: (i) a reduced expression, activity function and/or stability of the IGSF11 or domain (or variant), in said first cell contacted with the candidate compound compared to said first cell not contacted with said candidate compound; and/or (ii) an enhancement of the cell-mediated immune response against the first cell contacted with the candidate compound compared to the cell-mediated immune response against the first cell not contacted with the candidate compound; indicates that the candidate compound is a compound suitable for the treatment of a disease, disorder or condition such as a proliferative disorder or an infectious disease (preferably, a proliferative disorder such as a cancer); or
wherein: (i) an enhanced expression, activity function and/or stability of the IGSF11 or domain (or variant), in said first cell contacted with the candidate compound compared to said first cell not contacted with said candidate compound; and/or (ii) an reduction of the cell-mediated immune response against the first cell contacted with the candidate compound compared to the cell-mediated immune response against the first cell not contacted with the candidate compound; indicates that the candidate compound is a compound suitable for the treatment of a disease, disorder or condition such as autoimmunity, allergy or inflammatory conditions.

In certain embodiments of such aspects, the methods also include the step of providing (such as by obtaining) the first cell and/or the candidate compound and/or (components of) the cell-mediated immune response (preferably wherein the cell-mediated immune response comprises immune cells selected from the group consisting of: lymphocytes, T-cells, CTLs and TILs), in particular where each of such steps is conducted prior to the contacting step.

The reduction (or enhancement) of expression, activity function and/or stability or IGSF11 or of an IgC2 (or IgV) domain of IGSF11 (or variant thereof), or the enhancement (or reduction) of the cell-mediated immune response is, preferably, identified by reference to a control method. In one example, the control method may be one practiced in the absence of any candidate compound, or with compound having a known effect on such expression, function, activity and/or stability (such as a positive or negative control), and/or one practiced in the absence of (one or more components of) a cell-mediated immune response.

In particular of such embodiments, the compound having a known effect on such expression, function, activity and/or stability on the IGSF11, domain or the variant is an ABP of the invention (or a product or another modulating compound of the invention).

In certain embodiments of the screening method, the (components of) cell-mediated immune response is a second cell which is a cytotoxic immune cell, for example a cytotoxic T-lymphocyte (CTL), capable of immunologically recognising the first cell. Accordingly, the containing step of such embodiment comprises bringing into contact a first cell and the candidate compound and a second cell, wherein the first cell expresses IGSF11 or an IgC2 (or IgV) domain of IGSF11, or a variant thereof (eg, a protein or mRNA of the IGSF11 or variant) and the second cell is a cytotoxic immune cell, for example a cytotoxic T-lymphocyte (CTL), capable of immunologically recognising the first cell.

In related but alternative embodiments of the screening method, the (components of) cell-mediated immune response is a cell-free medium that has previously contained immunologically stimulated immune cells, for example cytotoxic T-lymphocytes (CTLs). Such immune cells may be stimulated by samples of the first cell and/or by polyclonal stimulants such as CD3-CD28 bead stimulation. Accordingly, the contacting step of such embodiment comprises bringing into contact a first cell and the candidate compound and a cell-free medium, wherein the first cell expresses IGSF11 or an IgC2 (or IgV) domain of IGSF11 (eg, a protein or mRNA of IGSF11 or of an IgC2 (or IgV) domain of IGSF11) and the cell-free medium had previously contained immunologically stimulated immune cells, for example a cytotoxic T-lymphocyte (CTL), such as those capable of immunologically recognising the first cell.

The first cell is preferably a cell involved with a proliferative disorder (such as a tumour), eg a cell derived from a tumour. The tumour or cell thereof, may be one or, or derived from, one of the tumours described elsewhere herein.

The candidate compound used in the screening methods may be one selected from a polypeptide, peptide, glycoprotein, a peptidomimetic, an antibody or antibody-like molecule (such as an intra-body); a nucleic acid such as a DNA or RNA, for example an antisense DNA or RNA, a ribozyme, an RNA or DNA aptamer, siRNA, shRNA and the like, including variants or derivatives thereof such as a peptide nucleic acid (PNA); a genetic construct for targeted gene editing, such as a CRISPR/Cas9 construct and/or guide RNA/DNA (gRNA/gDNA) and/or tracrRNA; a hetero bi-functional compound such as a PROTAC or HyT molecule; a carbohydrate such as a polysaccharide or oligosaccharide and the like, including variants or derivatives thereof; a lipid such as a fatty acid and the like, including variants or derivatives thereof; or a small organic molecules including but not limited to small molecule ligands, or small cell-permeable molecules.

In particular embodiments, the candidate compound is an ABP, such as one described elsewhere herein.

In certain embodiments of such screening aspects, the (candidate) compound, such as an ABP, is identified and/or characterised (as one) for use in medicine. For example, such screening methods may be practiced with the purpose of identifying and/or characterising a compound (such as an ABP) having properties suitable for therapeutic use.

In those methods described herein for identifying and/or characterising a compound, or for producing a compound, (in each case, such as an ABP) for use in medicine, any of such methods may comprise one or more further steps of determining (or of having determined) whether such (candidate) compound has one or more (functional) characteristics, such as any of those described elsewhere herein. For example, such methods may include a step of determining (or of having determined) whether such (candidate) compound is able to induce internalisation (eg induces internalisation, or internalises) IGSF11 protein from the surface of cells (such as tumour cells) that express IGSF11. In another example, such methods may include a step of determining (or of having determined) whether such (candidate) compound is able to enhance or increase killing and/or lysis of tumour cells, preferably cancer cells or cells; and in particular of whether such (candidate) compound is an anti-tumour ABP and/or is able to inhibit tumour growth in-vivo, preferably in a murine model of cancer (such as in a murine model of cancer described herein). A (candidate) compound—such as an ABP—determined to have such (functional) characteristic (or characteristcs), may thereby be determined as one that is for use in medicine.

The terms “of the [present] invention” “in accordance with the invention” “according to the invention” and the like, as used herein are intended to refer to all aspects and embodiments of the invention described and/or claimed herein.

As used herein, the term “comprising” is to be construed as encompassing both “including” and “consisting of”, both meanings being specifically intended, and hence individually disclosed embodiments in accordance with the present invention. Where used herein, “and/or” is to be taken as specific disclosure of each of the two specified features or components with or without the other. For example, “A and/or B” is to be taken as specific disclosure of each of (i) A, (ii) B and (iii) A and B, just as if each is set out individually herein. In the context of the present invention, the terms “about” and “approximately” denote an interval of accuracy that the person skilled in the art will understand to still ensure the technical effect of the feature in question. The term typically indicates deviation from the indicated numerical value by ±20%, ±15%, ±10%, and for example ±5%. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. As will be appreciated by the person of ordinary skill, the specific such deviation for a numerical value for a given technical effect will depend on the nature of the technical effect. For example, a natural or biological technical effect may generally have a larger such deviation than one for a man-made or engineering technical effect. Where an indefinite or definite article is used when referring to a singular noun, e.g. “a”, “an” or “the”, this includes a plural of that noun unless something else is specifically stated.

It is to be understood that application of the teachings of the present invention to a specific problem or environment, and the inclusion of variations of the present invention or additional features thereto (such as further aspects and embodiments), will be within the capabilities of one having ordinary skill in the art in light of the teachings contained herein.

Unless context dictates otherwise, the descriptions and definitions of the features set out above are not limited to any particular aspect or embodiment of the invention and apply equally to all aspects and embodiments which are described.

All references, patents, and publications cited herein are hereby incorporated by reference in their entirety.

### Certain Numbered Embodiments of the Present Invention

In view of the above, it will be appreciated that the present invention also relates to the following itemised embodiments:

Item 1. An isolated antigen binding protein (ABP) which specifically binds to a C2-type immunoglobulin-like (IgC2) domain of IGSF11 (VSIG3) protein (or, in another aspect, specifically binds to a V-type immunoglobulin-like (IgV) domain of IGSF11 (VSIG3) protein) or a variant thereof, and wherein the isolated ABP comprises at least one complementarity determining region (CDR) and, optionally, is able to inhibit the binding of an interacting protein to IGSF11 protein or to an IgC2 domain of IGSF11 protein (or, in the other aspect, to an IgV domain of IGSF11 protein) or, in either case, a variant thereof,

with the proviso that the ABP is not one or more of:
(A) one or more of an antibody (for example that binds to an IgC2 domain of IGSF11 protein (or, in the alternative aspect, for example that binds to an IgV domain of IGSF11 protein)), or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown selected from any of the variable chain combinations Chains-A-001 to Chains-A-037 as described in Table C; and/or
(B) one or more of an antibody (for example that binds to an IgC2 domain of IGSF11 protein (or, in the alternative aspect, for example that binds to an IgV domain of IGSF11 protein)), or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown selected from any of the variable chain combinations Chains-B-001 to Chains-B-008 as described in Table C.1.
   Item 1a. The isolated ABP of item 1, wherein the antibody heavy chain sequence and/or the antibody light chain sequence of the antibody or the antigen binding fragment thereof of proviso (A) and/or (B), in each case independently, has no more than fifteen, fourteen, thirteen, twelve or eleven (eg, for variable light chain), such with no more than ten, nine, eight, seven, six, five, four, preferably no more than three, two or one, amino acid substitution(s), insertion(s) or deletion(s) (in particular, substitution(s)) compared to the antibody heavy chain sequence and/or antibody light chain sequences set forth in item 1.
   Item 1b. The isolated ABP of item 1 or 1a, wherein the ABP is not one or more of:
(C) an antibody that (for example binds to an IgC2 domain of IGSF11 protein (or, in the alternative aspect, that for example binds to an IgV domain of IGSF11 protein)) and is selected from the list consisting of antibodies: #774206, #774208, #774213, #774221, #774226, #973401, #973408, #973422, #973428, #973433 and #973435, each as disclosed in WO 2018/027042 A1 as described in Table D, or an antigen binding fragment thereof.
   Item 1c. The isolated ABP of any one of items 1 to 1b, wherein the ABP is not one or more of:
(D) one or more of an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise complementarity determining region (CDR) CDR1 to CDR3 sequences in a combination selected from any of the following combinations of heavy and/or light chain CDRs, CDRs-A-001 to CDRs-A-037 as described in Table B; and/or
(E) one or more of an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise CDR1 to CDR3 sequences in a combination selected from any of the following combinations of heavy and/or light chain CDRs, CDRs-B-001 to CDRs-B-008 as described in Table B.1.
   Item 1d. The isolated ABP of item 1c, wherein the sequence of each CDR of the antibody or the antigen binding fragment thereof of proviso (D) and/or (E), comprises, in each case independently, no more than five or four (eg, for L-CDR1), or with no more than three or two, preferably no more than one, amino acid substitution(s), insertion(s) or deletion(s) (in particular, substitution(s)) compared to the CDR sequences set forth in item 1c.
   Item 2. The isolated ABP of any one of items 1 to 1d, wherein the ABP is not one or more of:
(F) one or more of an ABP comprising at least one complementarity determining region 3 (CDR3) having an amino acid sequence selected from SEQ ID Nos. 3, 7, 13, 17, 23, 27, 33, 37, 43, 47, 53, 57, 63, 67, 73, 77, 83, 87, 93, 97, 103, 107, 113, 117, 123, 127, 133, 137, 143, 147, 153, 157, 163, 167, 173, 177, 183, 187, 193, 197, 203, 207, 213, 217, 223, 227, 233, 237, 243, 247, 253, 257, 263, 267, 273, 277, 283, 287, 293, 297, 303, 307, 313, 317, 323, 327, 333, 337, 343, 347, 353, 357, 363, and 367.
   Item 2a. The isolated ABP of item 2, wherein the amino acid sequence of the CDR3 of the ABP of proviso (F) has at least 90% sequence identity to, or has no more than three or two, preferably no more than one amino acid substitution(s), deletion(s) or insertion(s) compared to, a CDR3 sequence set forth in item 2.
   Item 2b. The isolated ABP of any one of items 1 to 2a that does not bind (eg, does not substantially, appreciably or detectable bind) to an IgV domain of IGSF11 protein (or, in the other aspect, that does not bind to an IgC2 domain of IGSF11 protein) or a variant of such domain.
   Item 2c. The isolated ABP of any one of items 1 to 2b, wherein the interacting protein is: (i) an endogenous binding partner of IGSF11 protein; or (ii) a biochemical binding partner of IGSF11 protein. Item 2d. The isolated ABP of any one of items 1 to 2c comprising at least one CDR3 having an amino acid sequence with at least 900/sequence identity to, or having no more than three or two, preferably no more than one amino acid substitution(s), deletion(s) or insertion(s) compared to, a sequence selected from SEQ ID Nos.: 683, 687, 693, 697, 703, 707, 713, 717, 723, 727, 733, 737, 743, 747, 753, 757, 763, 767, 773, 777, 783, 787, 793, 797, 803, 807, 813, 817, 823, 827, 833, 837, 843, 847, 853, 857, 863, 867, 873, 877, 883, 887, 893, 897, 903, 907, 913, 917, 923, 927, 933, 937, 943, 947, 953, 957, 963, 967, 973, 977, 983, 987, 993, 997, 1003, 1007, 1013, 1017, 1023, 1027, 1033, 1037, 1043, 1047, 1053, 1057, 1063, and 1067.
   Item 2e. The isolated ABP of any one of items 1 to 2d, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise CDR1 to CDR3 sequences in a combination selected from any of the following combinations of heavy and/or light chain CDRs, CDRs-D-101 to CDRs-D-116 and CDRs-D-201 to CDRs-D-223:

| Combination | Heavy Chain CDR1-CDR3 | | | Light Chain CDR1-CDR3 | | |
|---|---|---|---|---|---|---|
| (ID) | (SEQ ID NO) | | | (SEQ ID NO) | | |
| CDRs-D-101 | 681 | 682 | 683 | 685 | 686 | 687 |
| CDRs-D-102 | 691 | 692 | 693 | 695 | 696 | 697 |
| CDRs-D-103 | 701 | 702 | 703 | 705 | 706 | 707 |
| CDRs-D-104 | 711 | 712 | 713 | 715 | 716 | 717 |
| CDRs-D-105 | 721 | 722 | 723 | 725 | 726 | 727 |
| CDRs-D-106 | 731 | 732 | 733 | 735 | 736 | 737 |
| CDRs-D-107 | 741 | 742 | 743 | 745 | 746 | 747 |
| CDRs-D-108 | 751 | 752 | 753 | 755 | 756 | 757 |
| CDRs-D-109 | 761 | 762 | 763 | 765 | 766 | 767 |
| CDRs-D-110 | 771 | 772 | 773 | 775 | 776 | 777 |
| CDRs-D-111 | 781 | 782 | 783 | 785 | 786 | 787 |
| CDRs-D-112 | 791 | 792 | 793 | 795 | 796 | 797 |
| CDRs-D-113 | 801 | 802 | 803 | 805 | 806 | 807 |
| CDRs-D-114 | 811 | 812 | 813 | 815 | 816 | 817 |
| CDRs-D-115 | 821 | 822 | 823 | 825 | 826 | 827 |
| CDRs-D-116 | 831 | 832 | 833 | 835 | 836 | 837 |
| CDRs-D-201 | 841 | 842 | 843 | 845 | 846 | 847 |
| CDRs-D-202 | 851 | 852 | 853 | 855 | 856 | 857 |
| CDRs-D-203 | 861 | 862 | 863 | 865 | 866 | 867 |
| CDRs-D-204 | 871 | 872 | 873 | 875 | 876 | 877 |
| CDRs-D-205 | 881 | 882 | 883 | 885 | 886 | 887 |
| CDRs-D-206 | 891 | 892 | 893 | 895 | 896 | 897 |
| CDRs-D-207 | 901 | 902 | 903 | 905 | 906 | 907 |
| CDRs-D-208 | 911 | 912 | 913 | 915 | 916 | 917 |
| CDRs-D-209 | 921 | 922 | 923 | 925 | 926 | 927 |
| CDRs-D-210 | 931 | 932 | 933 | 935 | 936 | 937 |
| CDRs-D-211 | 941 | 942 | 943 | 945 | 946 | 947 |
| CDRs-D-212 | 951 | 952 | 953 | 955 | 956 | 957 |
| CDRs-D-213 | 961 | 962 | 963 | 965 | 966 | 967 |
| CDRs-D-214 | 971 | 972 | 973 | 975 | 976 | 977 |
| CDRs-D-215 | 981 | 982 | 983 | 985 | 986 | 987 |
| CDRs-D-216 | 991 | 992 | 993 | 995 | 996 | 997 |
| CDRs-D-217 | 1001 | 1002 | 1003 | 1005 | 1006 | 1007 |
| CDRs-D-218 | 1011 | 1012 | 1013 | 1015 | 1016 | 1017 |
| CDRs-D-219 | 1021 | 1022 | 1023 | 1025 | 1026 | 1027 |
| CDRs-D-220 | 1031 | 1032 | 1033 | 1035 | 1036 | 1037 |
| CDRs-D-221 | 1041 | 1042 | 1043 | 1045 | 1046 | 1047 |
| CDRs-D-222 | 1051 | 1052 | 1053 | 1055 | 1056 | 1057 |
| CDRs-D-223 | 1061 | 1062 | 1063 | 1065 | 1066 | 1067 |

in each case independently, optionally with no more than three or two, preferably no more than one, amino acid substitution(s), insertion(s) or deletion(s) compared to these sequences.
   Item 2f. The isolated ABP of any one of items 1 to 2e, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences each comprises heavy chain CDR1 to CDR3 sequences in the combination CDRs-D-114 or CDRs-D-222, in each case independently, optionally with no more than one amino acid substitution(s), insertion(s) or deletion(s) compared to these sequences, and preferably wherein the ABP is able to inhibit the binding of the interacting protein to IGSF11 protein or to the IgC2 domain of IGSF11 protein or, in either case, a variant thereof, with an IC50 of 50 nM or 10 nM, or 0.5 nM or less, preferably as measured according to example 13 herein.
   Item 2g. The isolated ABP of any one of items 1 to 2f, wherein the ABP is an antibody, or an antigen binding fragment thereof, composed of at least one, preferably two, antibody heavy chain sequences, and at least one, preferably two, antibody light chain sequences, wherein at least one, preferably both, of the antibody light chain sequences each comprises heavy chain CDR1 to CDR3 sequences in the combination CDRs-D-114 or CDRs-D-222, in each case independently, optionally with no more than one amino acid substitution(s), insertion(s) or deletion(s) compared to these sequences, and preferably wherein the ABP is able to inhibit the binding of the interacting protein to IGSF11 protein or to the IgC2 domain of IGSF11 protein or, in either case, a variant thereof, with an IC50 of 50 nM or 10 nM, or 0.5 nM or less, preferably as measured according to example 13 herein.
   Item 2h. The isolated ABP of any one of items 1 to 2h, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences each comprises heavy chain CDR1 to CDR3 sequences in the combination CDRs-D-114 or CDRs-D-222, and at least one, preferably both, of the antibody light chain sequences each comprises light chain CDR1 to CDR3 sequences in the combination, respectively, CDRs-D-114 or CDRs-D-222, in each case independently, optionally with no more than one amino acid substitution(s), insertion(s) or deletion(s) compared to these sequences, and preferably wherein the ABP is able to inhibit the binding of the interacting protein to IGSF11 protein or to the IgV domain of IGSF11 protein or, in either case, a variant thereof, with an IC50 of 50 nM or 10 nM, or 0.5 nM or less, preferably as measured according to example 13 herein.
   Item 3. The isolated ABP of any one of items 1 to 2c comprising at least one CDR3 having an amino acid sequence with at least 90% sequence identity to, or having no more than three or two, preferably no more than one amino acid substitution(s), deletion(s) or insertion(s) compared to, a sequence selected from SEQ ID Nos.: 403, 407, 413, 417, 423, 427, 433, 437, 443, 447, 483, 487, 493, 497, 513, 517, 523, 527, 533, 537, 563, 567, 593, 597, 603, 607, 613 and 617 (or, in the other aspect, compared to, a sequence selected from SEQ ID Nos: 393, 397, 453, 457, 463, 467, 473, 477, 543, 547, 553, 557, 623, 627, 633, 637, 643 and 647).
   Item 3a. The isolated ABP of any one of items 1 to 2c and 3, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise CDR1 to CDR3 sequences in a combination selected from any of the following combinations of heavy and/or light chain CDRs, CDRs-C-001 to CDRs-C-029:

| | Heavy Chain | | | Light Chain | | |
|---|---|---|---|---|---|---|
| Combination | CDR1 to CDR3 | | | CDR1 to CDR3 | | |
| (ID) | (SEQ ID NO) | | | (SEQ ID NO) | | |
| CDRs-C-001 | 391 | 392 | 393 | 395 | 396 | 397 |
| CDRs-C-002 | 401 | 402 | 403 | 405 | 406 | 407 |
| CDRs-C-003 | 411 | 412 | 413 | 415 | 416 | 417 |
| CDRs-C-004 | 421 | 422 | 423 | 425 | 426 | 427 |
| CDRs-C-005 | 431 | 432 | 433 | 435 | 436 | 437 |
| CDRs-C-006 | 441 | 442 | 443 | 445 | 446 | 447 |
| CDRs-C-007 | 451 | 452 | 453 | 455 | 456 | 457 |
| CDRs-C-008 | 461 | 462 | 463 | 465 | 466 | 467 |
| CDRs-C-009 | 471 | 472 | 473 | 475 | 476 | 477 |
| CDRs-C-010 | 481 | 482 | 483 | 485 | 486 | 487 |
| CDRs-C-011 | 491 | 492 | 493 | 495 | 496 | 497 |
| CDRs-C-012 | 501 | 502 | 503 | 505 | 506 | 507 |
| CDRs-C-013 | 511 | 512 | 513 | 515 | 516 | 517 |
| CDRs-C-014 | 521 | 522 | 523 | 525 | 526 | 527 |
| CDRs-C-015 | 531 | 532 | 533 | 535 | 536 | 537 |
| CDRs-C-016 | 541 | 542 | 543 | 545 | 546 | 547 |
| CDRs-C-017 | 551 | 552 | 553 | 555 | 556 | 557 |
| CDRs-C-018 | 561 | 562 | 563 | 565 | 566 | 567 |
| CDRs-C-019 | 571 | 572 | 573 | 575 | 576 | 577 |
| CDRs-C-020 | 581 | 582 | 583 | 585 | 586 | 587 |
| CDRs-C-021 | 591 | 592 | 593 | 595 | 596 | 597 |
| CDRs-C-022 | 601 | 602 | 603 | 605 | 606 | 607 |
| CDRs-C-023 | 611 | 612 | 613 | 615 | 616 | 617 |
| CDRs-C-024 | 621 | 622 | 623 | 625 | 626 | 627 |
| CDRs-C-025 | 631 | 632 | 633 | 635 | 636 | 637 |
| CDRs-C-026 | 641 | 642 | 643 | 645 | 646 | 647 |
| CDRs-C-027 | 651 | 652 | 653 | 655 | 656 | 657 |
| CDRs-C-028 | 661 | 662 | 663 | 665 | 666 | 667 |
| CDRs-C-029 | 671 | 672 | 673 | 675 | 676 | 677 |

in each case independently, optionally with no more than three or two, preferably no more than one, amino acid substitution(s), insertion(s) or deletion(s) compared to these sequences.
   Item 4. The isolated ABP of any one of items 1 to 2c and items 3 or 3a, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences each comprises heavy chain CDR1 to CDR3 sequences in the combination CDRs-C-003 or CDRs-C-004, or in the combination CDRs-C-005, and at least one, preferably both, of the antibody light chain sequences each comprises light chain CDR1 to CDR3 sequences in the combination, respectively, CDRs-C-003 or CDRs-C-004, or in the combination CDRs-C-005, in each case independently, optionally with no more than one amino acid substitution(s), insertion(s) or deletion(s) compared to these sequences, and preferably wherein the ABP is able to inhibit the binding of the interacting protein to IGSF11 protein or to the IgC2 domain of IGSF11 protein or, in either case, a variant thereof, with an IC50 of 50 nM or 10 nM or less.
   Item 4a. The isolated ABP of any one of items 1 to 2c and 3 or 3a, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences each comprises heavy chain CDR1 to CDR3 sequences in the combination C-001 or C-007, and at least one, preferably both, of the antibody light chain sequences each comprises light chain CDR1 to CDR3 sequences in the combination, respectively, C-001 or C-007, in each case independently, optionally with no more than one amino acid substitution(s), insertion(s) or deletion(s) compared to these sequences, and preferably wherein the ABP is able to inhibit the binding of the interacting protein to IGSF11 protein or to the IgV domain of IGSF11 protein or, in either case, a variant thereof, with an IC50 of 50 nM or 10 nM or less.
   Item 5. An isolated ABP which competes with an ABP as recited in any one of items 1 to 4a for binding to an IgC2 domain of IGSF11 protein (or, in another aspect, competes for binding to an IgV domain of IGSF11 protein) or a variant thereof, and, optionally, is able to inhibit the binding of an interacting protein to IGSF11 protein or to an IgC2 domain of IGSF11 protein (or, in the other aspect, to an IgV domain of IGSF11 protein) or, in each case, a variant thereof,

with the proviso that the isolated ABP is not one or more of:
any ABP the subject of proviso (A) of item 1;
any ABP the subject of proviso (B) of item 1;
any ABP the subject of proviso (C) of item 1b;
any ABP the subject of proviso (D) of item 1c;
any ABP the subject of proviso (E) of item 1c; and/or
any ABP the subject of proviso (F) of item 2.
   Item 5a. The isolated ABP of any one of items 1 to 5, wherein the interacting protein is VSIR (VISTA) protein or a variant thereof.
   Item 5b. The isolated ABP of any one of items 1 to 5a that is able to enhance or increase killing and/or lysis of cells expressing IGSF11 or an IgC2 domain (or an IgV domain) of IGSF11, or a variant thereof.
   Item 5c. The isolated ABP of any one of items 1 to 5b that is able to enhance or increase killing and/or lysis of tumour cells, preferably cancer cell or cells that originate from a tumour cell and/or cells that express IGSF11 or an IgC2 domain (or an IgV domain) of IGSF11, or a variant thereof.
   Item 5d. The isolated ABP of any one of items 1 to 5c that is an anti-tumour ABP.
   Item 5e. The isolated ABP of any one of items 1 to 5e that is able to inhibit tumour growth in-vivo, preferably in a murine model of cancer.
   Item 6. The isolated ABP of any one of items 1 to 5e that enhances killing and/or lysis of cells expressing IGSF11, or a variant of IGSF11, by cytotoxic T cells and/or TILs.
   Item 7. The isolated ABP of any one of items 1 to 6 that (i) enhances a cell-mediated immune response, such as that mediated by an activated cytotoxic T-cell (CTL), to a mammalian cell expressing said IGSF11 or the variant of IGSF11; and/or (ii) increases immune cell, such as T-cell, activity and/or survival in the presence of a mammalian cell expressing said IGSF11 or the variant of IGSF11.
   Item 7a. The isolated ABP of any one of items 1 to 7 that modifies the microenvironment of a tumour, in particular modulates the number and/or type of immune cells present in the tumour, and more suitably reduces the number of intra-tumoural myeloid-derived suppressor cells (MDSCs) and/or increases the number of intra-tumoural CTLs.
   Item 7b. The isolated ABP of any one of items 1 to 7a that decreases (the number of M2) tumour-associated macrophages (TAMs) and/or increases the number of (intra-tumoural) CTLs, optionally, in each case, within the tumour microenvironment.
   Item 7c. The isolated ABP of any one of items 1 to 7b, wherein the ABP is able to inhibit the binding of an interacting protein to IGSF11 protein or to an IgC domain or (an IgV domain) of IGSF11 protein or, in either case, a variant thereof; optionally with an IC50 of 50 nM or 10 nM or less.
   Item 7d. The isolated ABP of any one of items 1 to 7c, wherein the ABP does not inhibit the interaction between VSIR (VISTA) protein or a variant thereof and IGSF11 protein or the IgC2 domain (or IgV domain) of IGSF11 protein or a variant thereof.
   Item 8. The isolated ABP of any one of items 1 to 7d that is an antibody or an antigen binding fragment thereof, wherein the antibody is a monoclonal antibody, or wherein the antigen binding fragment is a fragment of a monoclonal antibody.
   Item 9. The isolated ABP of any one of items 1 to 8 that is an antibody or an antigen binding fragment thereof, wherein the antibody is a human antibody a humanised antibody or a chimeric-human antibody, or wherein the antigen binding fragment is a fragment of a human antibody a humanised antibody or a chimeric-human antibody.
   Item 9a. The isolated ABP of any one of items 1 to 9 that is multi-specific, in particular is bi-specific (such as a bispecific T-cell engager (BiTE) ABP or antibody).
   Item 10. An isolated nucleic acid encoding for an ABP, or for an antigen binding fragment or a monomer of an ABP, wherein the ABP is one of any one of items 1 to 9a.
   Item 11. A recombinant host cell comprising a nucleic acid recited in item 10.
   Item 12. A pharmaceutical composition comprising:

(X):
(i) an ABP of any one of items 1 to 9a; or
(ii) a nucleic acid recited in item 10 or a recombinant host cell of item 11, in particular a T cell comprising a nucleic acid expressing an ABP comprising a chimeric antigen receptor (CAR); or
(iii) a compound that is an inhibitor of the expression, function, activity and/or stability of immunoglobulin superfamily member 11 (IGSF11, or VSIG3), or of a C2-type immunoglobulin-like (IgC2) domain of IGSF11 (or, in another aspect, is an inhibitor of the expression, function, activity and/or stability of a V-type immunoglobulin-like (IgV) domain of IGSF11) or of a variant thereof,
with the proviso that the compound is not one or more of:
   any ABP the subject of proviso (A) of item 1;
   any ABP the subject of proviso (B) of item 1;
   any ABP the subject of proviso (C) of item 1b;
   any ABP the subject of proviso (D) of item 1c;
   any ABP the subject of proviso (E) of item 1c; and/or
   any ABP the subject of proviso (F) of item 2, and

(Y):
a pharmaceutically acceptable carrier, stabiliser and/or excipient.
   Item 13. A product for use in medicine, wherein the product is selected from the list consisting of:
(i) an isolated ABP of any one of items 1 to 9a, and
(ii) an isolated nucleic acid recited in item 10 or a recombinant host cell of item 11, in particular T cell comprising a nucleic acid expressing an ABP comprising a chimeric antigen receptor (CAR), and
(iii) a compound that is an inhibitor of the expression, function, activity and/or stability of immunoglobulin superfamily member 11 (IGSF11, or VSIG3), or of a C2-type immunoglobulin-like (IgC2) domain of IGSF11 (or, in another aspect, is an inhibitor of the expression, function, activity and/or stability of a V-type immunoglobulin-like (IgV) domain of IGSF11 (VSIG3)) or of a variant thereof,
with the proviso that the compound is not one or more of:
   any ABP the subject of proviso (A) of item 1;
   any ABP the subject of proviso (B) of item 1;
   any ABP the subject of proviso (C) of item 1b;
   any ABP the subject of proviso (D) of item 1c;
   any ABP the subject of proviso (E) of item 1c; and/or
   any ABP the subject of proviso (F) of item 2.
      Item 14. The product for use in medicine of item 13 wherein the product is for use in the treatment of a proliferative disorder that is associated with the undesired presence of IGSF11-positive cells or cells positive for a variant of IGSF11 and/or that is associated with cellular resistance against a cell-mediated immune response and/or that is associated with expression or activity of IGSF11 or a variant thereof of IGSF11.
      Item 15. The product for use in medicine of item 14, wherein cells involved in the proliferative disorder are resistant to a cell-mediated immune response.
      Item 16. The product for use in medicine of any one of items 13 to 15, wherein the product is for use in enhancing an immune response in a mammalian subject, preferably for use in aiding a cell-mediated immune response in the subject such as the subject's T cell mediated immune response, for example for treating a proliferative disease, such as a cancer disease, or for treating an infectious disease.
      Item 17. The product for use in medicine of any one of items 13 to 16, wherein the product is for use in the treatment of a proliferative disorder resistant and/or refractory to PD1/PDL1 blockade therapy and/or to CTLA4 blockade therapy.
      Item 17a. The product for use in medicine of any one of items 13 to 16, wherein the product is for use in the treatment of a proliferative disorder in combination with a different anti-proliferative therapy.
      Item 17b. The product for use in medicine of any one of items 13 to 16, wherein the product is for use in the treatment of a cancer in combination with immunotherapy with a ligand to an immune checkpoint molecule.
      Item 17c. The product for use in medicine of item 17b, wherein the ligand is one that binds to an immune checkpoint molecule selected from the group consisting of: A2AR, B7-H3, B7-H4, CTLA-4, IDO, KIR, LAG3, PD-1 (or one of its ligands PD-L1 and PD-L2), TIM-3 (or its ligand galectin-9), TIGIT and VISTA.
      Item 17d. The product for use in medicine of item 17b or 17c, wherein the ligand binds to an immune checkpoint molecule selected from CTLA-4, PD-1 and PD-L1.
      Item 17e. The product for use in medicine of any one of items 17b to 17d, wherein the ligand is an antibody selected from the group consisting of: ipilimumab, nivolumab, pembrolizumab, BGB-A317, atezolizumab, avelumab and durvaluma; in particular an antibody selected from the group consisting of: ipilimumab (YERVOY), nivolumab (OPDIVO), pembrolizumab (KEYTRUDA) and atezolizumab (TECENTRIQ).
      Item 18. An in-vitro method for determining whether a subject has, or is at risk of, developing a disease, disorder or condition that is associated with the undesired presence of IGSF11-positive cells (or cells positive for a variant of IGSF11) and/or that is associated with cellular resistance against a cell-mediated immune response and/or that is associated with expression or activity of IGSF11 (or a variant thereof), the method comprising the step of:
detecting a C2-type immunoglobulin-like (IgC2) domain of IGSF11 (or, in another aspect, detecting a V-type immunoglobulin-like (IgV) domain of IGSF11) (or a variant of such domain), in particular the presence (or an amount) of or expression and/or activity of such domain of IGSF11 (or the variant thereof), in a biological sample from said subject,
   wherein the detection of such domain of IGSF11 (or the variant thereof) in the sample indicates such disease, disorder or condition, or a risk of developing such disease, disorder or condition, in the subject; and optionally, wherein such domain of the IGSF11 (or variant thereof) is detected with an ABP of any one of items 1 to 9a.
   Item 19. An in-vitro method for determining whether a subject has, or has a risk of developing, a disease, disorder or condition that is associated with the undesired presence of IGSF11-positive cells (or cells positive for a variant of IGSF11) and/or that is associated with cellular resistance against a cell-mediated immune response and/or that is associated with expression or activity of IGSF11 (or a variant thereof), the method comprising the steps of:
contacting cells of the subject involved with the disease, disorder or condition with an ABP of any one of items 1 to 9a, and/or with a product recited in any one of items 13 to 17e, in the presence of a cell-mediated immune response, preferably wherein the cell-mediated immune response comprises immune cells selected from the group consisting of: lymphocytes, T-cells, CTLs and TILs; and
determining the cell-mediated immune response against such cells of the subject, wherein an enhancement of the cell-mediated immune response against such cells of the subject indicates that the subject has or has a risk of developing a disease, disorder or condition that is selected from a proliferative disorder or an infectious disease.
   Item 20. An in-vitro method for identifying and/or characterising a compound suitable for the treatment of a disease, disorder or condition that is associated with the undesired presence of IGSF11-positive cells (or cells positive for a variant of IGSF11) and/or that is characterised by cellular resistance against a cell-mediated immune response and/or one that is characterised by expression or activity of IGSF11 (or a variant thereof), the method comprising the steps of:
(a) bringing into contact a first cell expressing a protein comprising a C2-type immunoglobulin-like (IgC2) domain of IGSF11 (or, in another aspect, expressing a protein comprising a V-type immunoglobulin-like (IgV) domain of IGSF11) (or a variant of such domain) and (x) the candidate compound, or (y) the candidate compound and a cell-mediated immune response, preferably wherein the cell-mediated immune response comprises immune cells selected from the group consisting of: lymphocytes, T-cells, CTLs and TILs; and
(b) determining (i) the expression, activity, function and/or stability of the (eg protein or mRNA of) such domain of IGSF11 (or variant), in the first cell; and/or (ii) the cell-mediated immune response against the first cell,
   wherein: (i) a reduced expression, activity function and/or stability of such domain of IGSF11 (or variant), in said first cell contacted with the candidate compound compared to said first cell not contacted with said candidate compound; and/or (ii) an enhancement of the cell-mediated immune response against the first cell contacted with the candidate compound compared to the cell-mediated immune response against the first cell not contacted with the candidate compound; indicates that the candidate compound is a compound suitable for the treatment of a disease, disorder or condition that is selected from a proliferative disorder or an infectious disease; and optionally, wherein the reduction of expression, activity function and/or stability of such domain of IGSF11 (eg, induction of internalisation of IGSF11 protein or such domain of IGSF11 protein) and/or the enhancement of the cell-mediated immune response is identified by reference to a control method practised with a compound having a known effect on such expression, function, activity and/or stability, in particular a positive or negative control; and wherein the compound having a known effect on such expression, function, activity and/or stability is an ABP of any one of items 1 to 9a and/or is a product recited in any one of items 13 to 17e.
   Item 20a. The method of item 20, wherein the protein expressed by the first cell does not comprise the IgV domain of IGSF11 (or, in the other aspect, does not comprise the IgC2 domain of IGSF).
   Item 21. A method for identifying and/or characterising an ABP as one specifically binding to a C2-type immunoglobulin-like (IgC2) domain of IGSF11 (VSIG3) protein (or, in another aspect, as one specifically binding to a V-type immunoglobulin-like (IgV) domain of IGSF11 (VSIG3) protein) or a variant thereof, the method comprising the step of:
detecting binding of the ABP to an epitope of (or comprised in) such domain of IGSF11 protein (or variant thereof),
   thereby identifying and/or characterising the ABP as one that specifically binds to the IgC2 domain of IGSF11 protein (or, in the other aspect as one that specifically binds to the IgV domain of IGSF11 protein), or variant thereof.
   Item 22. The method of item 21, further comprising the step of:
testing for binding of the ABP to an epitope of (or comprised in) an IgV domain of IGSF11 protein (or, in the other aspect, to an epitope of, or comprised in, an IgV domain of IGSF11 protein) or, optionally, a variant thereof, wherein, absence of detectable binding of the ABP to the epitope of (or comprised in) such domain of IGSF11 protein (or variant thereof) further characterises the ABP as one that specifically binds to the IgC2 domain of IGSF11 protein (or, in the other aspect as one that specifically binds to the IgV domain of IGSF11 protein) or variant thereof.
   Item 23. The method of item 21 or 22, wherein:
the detecting step of item 21 comprises detecting binding of the ABP to a first test protein, wherein the first test protein: (i) comprises the IgC2 domain of IGSF11 or a variant or fragment of such domain; and (ii) does not comprise an IgV domain of IGSF11 (or, in the other aspect: (i) comprises the IgV domain of IGSF11 or a fragment of such domain; and (ii) does not comprise an IgC2 domain of IGSF11) or, optionally, a variant thereof; and/or
the testing step of item 22 comprises testing for binding of the ABP to a second test protein, wherein the second test protein: (a) comprises the IgV domain of IGSF11 or a variant or fragment of such domain; and (b) does not comprise the IgC2 domain of IGSF11, or a variant or fragment of such domain (or, in the other aspect, (a) comprises the IgC2 domain of IGSF11 or a variant or fragment of such domain thereof; and (b) does not comprise the IgV domain of IGSF11, or a variant or fragment of such domain).
   Item 24. The method of item 23, wherein:
the first test protein does not comprise an IgV domain of IGSF11 (or, in the other aspect, does not comprise an IgC2 domain of IGSF11) or a variant or fragment of such domain; and/or
the second test protein comprises the IgV domain of IGSF11 (or, in the other aspect, comprises the IgC2 domain of IGSF11) or, optionally, a variant thereof.
   Item 25. The method of any one of items 21 to 24, wherein the ABP and the optional first test protein are provided prior to the detecting step and/or the ABP and the optional second test protein are provided prior to the testing step.
   Item 26. The method of any one of items 21 to 25, wherein the ABP that is identified and/or characterised as one that specifically binds to the IgC2 domain of IGSF11 protein (or, in the other aspect, as one that specifically binds to the IgV domain of IGSF11) or variant thereof is further (in particular, is thereby) identified and/or characterised as one for use in medicine.
   Item 26a. The method of any one of items 21 to 26, wherein the ABP is identified and/or characterised for use in medicine.
   Item 27. A method for identifying and/or characterising an ABP for use in medicine, the method comprising the steps of:
providing an ABP that binds to IGSF11 protein (or a variant thereof); and
identifying and/or characterising the provided ABP as one that specifically binds to an IgC2 domain of IGSF11 protein (or, in another aspect, as one that specifically binds to an IgV domain of IGSF11 protein) or a variant thereof,
   thereby identifying and/or characterising the ABP for use in medicine.
   Item 28. A method for producing an ABP for use in medicine, the method comprising the steps of:
providing a hybridoma or (host) cell capable of expressing an ABP that binds to IGSF11 protein (or a variant thereof), for example a recombinant cell line comprising at least one genetic construct comprising coding sequence(s) encoding said ABP; and
culturing said hybridoma or host cell under conditions that allow for the expression of the ABP;
optionally, isolating the ABP expressed by said hybridoma or host cell; and
identifying and/or characterising the expressed ABP as one that specifically binds to an IgC2 domain of IGSF11 protein (or, in another aspect, as one that specifically binds to an IgV domain of IGSF11 protein) or a variant thereof,
   thereby producing the ABP for use in medicine.
   Item 29. The method of item 27 or 28, wherein the identifying and/or characterising step comprises a method of any one of items 21 to 25.
   Item 29a. The method of any one of items 26a to 29, further comprising the step of: determining or having determined, that the ABP has one or more of the functional characteristics as set forth in any one of items 5b to 7d, preferably in any of items 5b to 5e; optionally, wherein an ABP determined to have one or more of such functional characteristics is for use in medicine.
   Item 30. A use of an IgC2 domain of IGSF11 protein (or, in another aspect, of an IgV domain of IGSF protein) or a variant or fragment (eg, at least one epitope) of such domain to identify, characterise and/or produce an ABP for use in medicine, suitably wherein the ABP specifically binds to such domain of IGSF11 protein (or variant thereof).
   Item 31. The use of item 30, further comprising the use of an IgV domain of IGSF11 protein (or, in the other aspect, the use of an IgC2 domain of IGSF11 protein) or, optionally, a variant thereof, suitably wherein the ABP does not bind to such domain of IGSF11 protein (or variant thereof).
   Item 32. The use of item 30 or 31, wherein the use comprises the use of:
a first test protein, wherein the test protein: (i) comprises the IgC2 domain of IGSF11 or a variant or fragment of such domain; and (ii) does not comprise an IgV domain of IGSF11 (or, in the other aspect, (i) comprises the IgV domain of IGSF11 or a variant or fragment of such domain; and (ii) does not comprise an IgC2 domain of IGSF11) or, optionally, a variant thereof; and/or
a second test protein, wherein the second test protein: (a) comprises an IgV domain of IGSF11 or a variant or fragment of such domain thereof; and (b) does not comprise the IgC2 domain of IGSF11, or a fragment of such domain (or, in the other aspect, (a) comprises an IgC2 domain of IGSF11 or a variant or fragment of such domain thereof; and (b) does not comprise the IgC2 domain of IGSF11, or a fragment of such domain) or, optionally, a variant thereof.
   Item 33. The use of item 32, wherein:
the first test protein does not comprise an IgV domain of IGSF11 or a variant or fragment of such domain (or, in the other aspect, does not comprise an IgC2 domain of IGSF11 or a variant or fragment of such domain); and/or
the second test protein comprises the IgV domain of IGSF11 (or, in the other aspect, comprises the IgC2 domain of IGSF11) or a variant thereof.
   Item 34. The method of any one of items 26 to 29, or the use of any one of items 30 to 33, wherein the ABP for use in medicine is:
an ABP for use in the treatment of a proliferative disorder that is associated with the undesired presence of IGSF11-positive cells or cells positive for a variant of IGSF11 and/or that is associated with cellular resistance against a cell-mediated immune response and/or that is associated with expression or activity of IGSF11 or a variant thereof of IGSF11, suitable wherein cells involved in the proliferative disorder are resistant to a cell-mediated immune response;
an ABP for use in enhancing an immune response in a mammalian subject, preferably for use in aiding a cell-mediated immune response in a subject such as the subject's T cell mediated immune response, for example for treating a proliferative disease, such as a cancer disease, of for treating an infectious disease; and/or
an ABP for use in the treatment of a proliferative disorder resistant and/or refractory to PD1/PDL1 and/or CTLA4 blockade therapy.
   Item 35. The method of any one of items 21 to 29 and 34, or the use of any one of items 30 to 34, wherein the ABP:
is capable of enhancing or increasing killing and/or lysis of cells expressing IGSF11 or an IgC2 domain (or IgV domain) of IGSF11, or a variant thereof;
is capable of enhancing or increasing killing and/or lysis of tumour cells, preferably cancer cell or cells that originate from a tumour cell and/or cells that express IGSF11 or an IgC2 domain (or IgV domain) of IGSF11, or a variant thereof;
is a therapeutic antibody able to treat, ameliorate and/or delay progression of a disease, disorder or condition, in particular a disease, disorder or condition mentioned herein elsewhere;
is an anti-tumour antibody;
is capable of inhibiting tumour growth in-vivo, preferably in a murine model of cancer;
is able to inhibit the binding of an interacting protein to IGSF11 protein or a variant thereof, suitably: (i) wherein the interacting protein is VSIR (VISTA) protein or a variant thereof; or, alternatively (ii) wherein the interacting protein is not VSIR (VISTA) protein or a variant thereof;
is able to inhibit (eg, inhibits) the interaction between VSIR (VISTA) protein or a variant thereof and the IgC2 domain (or the IgV domain) of IGSF11 protein or a variant thereof or, alternatively (ii) is not able to inhibit (eg, does not inhibit) the interaction between VSIR (VISTA) protein or a variant thereof and the IgC2 domain (or the IgV domain) of IGSF11 protein or a variant thereof;
enhances killing and/or lysis of cells expressing IGSF11, or a variant of IGSF11, by cytotoxic T cells and/or TIL;
enhances a cell-mediated immune response, such as that mediated by an activated cytotoxic T-cell (CTL), to a mammalian cell expressing said IGSF11 or the variant of IGSF11;
increases immune cell, such as T-cell, activity and/or survival in the presence of a mammalian cell expressing said IGSF11 or the variant of IGSF11;
modifies the microenvironment of a tumour, suitably increases the number and/or type of immune cells present in the tumour, and more suitably reduces the number of intra-tumoural MDSCs and/or increases the number of intra-tumoural CTLs;
recruits and/or activates NK cells and/or mediates antibody-dependent cellular cytotoxicity (ADCC);
recruits and/or activates macrophages and/or mediates antibody-dependent cellular phagocytosis (ADCP);
recruits complement and/or mediates complement dependent cytotoxicity (CDC); and/or
decreases (the number of) M2 tumour-associated macrophages (TAMs) and/or increases the number of (intra-tumoural) CTLs, optionally, in each case, within the tumour microenvironment: and/or
induces internalisation of IGSF11 protein from the surface of cells (such as tumour cells that express IGSF11).
   Item 36. The method of any one of items 21 to 29, 34 and 35, or the use of any one of items 30 to 35, wherein the ABP is an antibody, or an antigen binding fragment thereof.
   Item 37. The method or use of item 36, wherein the antibody is a monoclonal antibody, or wherein the antigen binding fragment is a fragment of a monoclonal antibody.
   Item 38. The method or use of item 36 or 37, wherein the antibody is a human antibody a humanised antibody or a chimeric-human antibody, or wherein the antigen binding fragment is a fragment of a human antibody a humanised antibody or a chimeric-human antibody.
   Item 39. A method for inhibiting the interaction between IGSF11 protein and an interacting protein of IGSF11 protein, such as an interacting protein that binds to an IgC2 domain of IGSF11 protein (or, in another aspect, that binds to an IgV domain of IGSF11 protein) or a variant thereof, the method comprising the step of:
exposing IGSF11 protein (or a variant thereof) to a compound that is an inhibitor of the expression, function, activity and/or stability of an IgC2 domain of IGSF11 protein (or, in the other aspect, is an inhibitor of the expression, function, activity and/or stability of an IgV domain of IGSF11 protein) or a variant thereof,

with the proviso that the compound is not one or more of:
any ABP the subject of proviso (A) of item 1;
any ABP the subject of proviso (B) of item 1;
any ABP the subject of proviso (C) of item 1b;
any ABP the subject of proviso (D) of item 1c;
any ABP the subject of proviso (E) of item 1c; and/or
any ABP the subject of proviso (F) of item 2,
   thereby, inhibiting the interaction between IGSF11 protein and an interacting protein of IGSF11 protein.
   Item 39a. The method of item 39, as an in-vitro method
   Item 40. A method for treating a subject in need thereof, said treatment comprising inhibiting the interaction between IGSF11 protein and an interacting protein of IGSF11 protein, such as an interacting protein that binds to an IgC2 domain of the IGSF11 protein (or, in another aspect, that binds to an IgV domain of IGSF11 protein), the method comprising the step of:
administering to the subject a (eg, therapeutically effective amount of a) compound that is an inhibitor of the expression, function, activity and/or stability of an IgC2 domain of IGSF11 protein (or, in the other aspect, that is an inhibitor of the expression, function, activity and/or stability of an IgV domain of IGSF11 protein) or a variant thereof,

with the proviso that the compound is not one or more of:
any ABP the subject of proviso (A) of item 1;
any ABP the subject of proviso (B) of item 1;
any ABP the subject of proviso (C) of item 1b;
any ABP the subject of proviso (D) of item 1c;
any ABP the subject of proviso (E) of item 1c; and/or
any ABP the subject of proviso (F) of item 2,
   to inhibit the interaction between IGSF11 protein and an interacting protein of IGSF11 protein.
   Item 41. The method of any one of items 30 to 40, wherein the compound is an ABP of any one of items 1 to 9a.
   Item 42. The method of any one of items 39 to 41, wherein the interacting protein of IGSF11 protein is an endogenous binding partner of IGSF11 protein.
   Item 43. The method of any one of items 39 to 43, wherein the interacting protein of IGSF11 protein is VSIR (VISTA) protein or a variant thereof.
   Item 44. A method for identifying, generating and/or producing an ABP that specifically binds to an IgC2 domain of IGSF11 (or to an IgV domain of IGSF11) or a variant thereof, the method comprising the use of such domain or an epitope of (or comprised in) such domain: (i) to screen a display library of a plurality of ABPs; or (ii) to immunise an animal.
   Item 45. The method of item 44, wherein the use comprises the use of a protein that comprises at least one epitope of (or comprised in) the IgC2 domain of IGSF11 (or variant thereof), wherein the protein does not comprise an IgV domain of IGSF11 (or a variant or epitope thereof) (or, wherein the use comprises the use of a protein comprising at least one epitope of (or comprised in) the IgV domain of IGSF11 (or variant or epitope thereof), wherein the protein does not comprise an IgC2 domain of IGSF11 (or a variant or epitope thereof)).
   Item 46. The method of item 44, wherein the use comprises the use of a nucleic acid that encodes a protein comprising at least one epitope of (or comprised in) the IgC2 domain of IGSF11 (or variant thereof), wherein the nucleic acid does not encode a protein comprising an IgV domain of IGSF11 (or a variant or epitope thereof thereof) (or, wherein the use comprises the use of a nucleic acid encoding a protein comprising at least one epitope of (or comprised in) the IgV domain of IGSF11 (or variant thereof), wherein the nucleic acid does not encode a protein comprising the IgC2 domain of IGSF11 (or variant or epitope thereof thereof)).
   Item 47. The method of item 44, comprising the step of immunising an animal (in particular a mammal, eg, a mouse, rat, rabbit, goat, camel, or llama) with a protein recited in item 45 or with the nucleic acid recited in item 46.
   Item 47a. The method of item 47, comprising a step of administering to the animal an immunisation composition comprising a protein recited in item 45 or a nucleic acid recited in item 46, and optionally together with a pharmaceutically acceptable carrier and/or excipient.
   Item 48. The method of item 47 or 47a, further comprising the step of isolating from the animal: (i) sera that comprises an ABP that specifically binds to said domain of IGSF11 (or variant thereof); and/or (ii) B cells that express an ABP that specifically binds to said domain of IGSF11 (or variant thereof).
   Item 49. The method of item 44, comprising the steps of screening a display library (eg, a phage display library) that displays a plurality of ABPs with a protein of item 45, and identifying an ABP that specifically binds to the said domain of IGSF11 (or variant thereof).
   Item 50. The method of item 48 or 49, further comprising the step of isolating (eg, purifying) the ABP that specifically binds to the said domain of IGSF11 (or variant thereof).
   Item 51. The method of any one of items 44 to 50, for identifying, generating and/or producing an ABP for use in medicine.
   Item 52. The method of item 51, further comprising the step of: determining or having determined, that the ABP has one or more of the functional characteristics as set forth in any one of items 5b to 7d, preferably in any of items 5b to 5e; optionally, wherein an ABP determined to have one or more of such functional characteristics is for use in medicine.

In addition, it will also be appreciated that the present invention also relates to the following further itemised embodiments:

Item A1. A method for identifying, generating and/or producing an ABP that specifically binds to a C2-type immunoglobulin-like (IgC2) domain of IGSF11 (VSIG3) protein or a variant thereof, the method comprising the use of such IgC2 domain of IGSF11 (or variant or epitope thereof): (i) to screen a display library of a plurality of ABPs; or (ii) to immunise an animal, in particular a mammal,
wherein, the use comprises the use of a protein that comprises at least one epitope of (or comprised in) the IgC2 domain of IGSF11 (or variant thereof) and does not comprise an IgV domain of IGSF11 or a variant or epitope thereof; or
wherein, the use comprises the uses of a nucleic acid that encodes a protein that comprises at least one epitope of (or comprised in) the IgC2 domain of IGSF11 (or variant thereof) and does not encode a protein that comprises an IgV domain of IGSF11 or a variant or epitope thereof.
   Item A2. The method of item A1, comprising the steps of:

(X):
screening a display library, in particular a phage display library, that displays a plurality of ABPs with the protein; and
identifying an ABP that specifically binds to the IgC2 domain of IGSF11 or variant thereof, or

(Y):
administering to the animal an immunisation composition comprising the protein or the nucleic acid, and optionally together with a pharmaceutically acceptable carrier and/or excipient; and
isolating from the animal: (i) sera that comprises an ABP that specifically binds to the IgC2 domain of IGSF11 or variant thereof; and/or (ii) B cells that express an ABP that specifically binds the IgC2 domain of IGSF11 or variant thereof, and
further comprising the step of isolating, in particular purifying, an ABP that specifically binds to the IgC2 domain of IGSF11 or variant thereof.
   Item A3. A method for identifying and/or characterising an ABP as one specifically binding to a C2-type immunoglobulin-like (IgC2) domain of IGSF11 (VSIG3) protein or a variant thereof, the method comprising the step of:
detecting binding of the ABP to an epitope of (or comprised in) the IgC2 domain of IGSF11 protein (or variant thereof),
thereby identifying and/or characterising the ABP as one that specifically binds to the IgC2 domain of IGSF11 protein, or variant thereof.
   Item A4. The method of item A3, further comprising the step of:
testing for binding of the ABP to an epitope of (or comprised in) an IgV domain of IGSF11 protein or, optionally, a variant thereof,
wherein, absence of detectable binding of the ABP to the epitope of (or comprised in) such IgV domain of IGSF11 protein (or variant thereof) further characterises the ABP as one that specifically binds to the IgC2 domain of IGSF11 protein, or variant thereof.
   Item A5. The method of item A3 or A4, wherein:
the detecting step of item A3 comprises detecting binding of the ABP to a first test protein, wherein the first test protein: (i) comprises the IgC2 domain of IGSF11 or a variant or fragment of such domain; and (ii) does not comprise the IgV domain of IGSF11 or, optionally, a variant thereof; and/or
the testing step of item A4 comprises testing for binding of the ABP to a second test protein, wherein the second test protein: (a) comprises the IgV domain of IGSF11 or a variant or fragment of such domain; and (b) does not comprise the IgC2 domain of IGSF11 or a variant or fragment of such domain
   Item A6. The method of item A5, wherein:
the first test protein does not comprise an IgV domain of IGSF11 or a variant or fragment of such domain; and/or
the second test protein comprises the IgV domain of IGSF11 or, optionally, a variant thereof.
   Item A7. The method of any one of items A1 to A6, wherein the ABP that that specifically binds to the IgC2 domain of IGSF11 a variant thereof is, in particular further and/or thereby identified and/or characterised as, one for use in medicine.
   Item A8. An isolated antigen binding protein (ABP) which specifically binds to a C2-type immunoglobulin-like (IgC2) domain of IGSF11 (VSIG3) protein or a variant thereof, and wherein the isolated ABP comprises at least one complementarity determining region (CDR) and, optionally, is able to inhibit the binding of an interacting protein to IGSF11 protein or to an IgC2 domain of IGSF11 protein or, in either case, a variant thereof,

with the proviso that the ABP is not one or more of:
(A) one or more of an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown selected from any of the variable chain combinations Chains-A-001 to Chains-A-037 as described in Table C; and/or
(B) one or more of an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown selected from any of the variable chain combinations Chains-B-001 to Chains-B-008 as described in Table C.1.
   Item A9. The isolated ABP of item A8, wherein the ABP is not one or more of:
(C) an antibody selected from the list consisting of antibodies: #774206, #774208, #774213, #774221, #774226, #973401, #973408, #973422, #973428, #973433 and #973435, each as described in Table D, or an antigen binding fragment thereof.
   Item A10. The isolated ABP of item A8 or A9, wherein the ABP is not one or more of:
(F) one or more of an ABP comprising at least one complementarity determining region 3 (CDR3) having an amino acid sequence selected from SEQ ID Nos. 3, 7, 13, 17, 23, 27, 33, 37, 43, 47, 53, 57, 63, 67, 73, 77, 83, 87, 93, 97, 103, 107, 113, 117, 123, 127, 133, 137, 143, 147, 153, 157, 163, 167, 173, 177, 183, 187, 193, 197, 203, 207, 213, 217, 223, 227, 233, 237, 243, 247, 253, 257, 263, 267, 273, 277, 283, 287, 293, 297, 303, 307, 313, 317, 323, 327, 333, 337, 343, 347, 353, 357, 363, and 367.
   Item A11. The isolated ABP of any one of items A8 to A10 comprising at least one CDR3 having an amino acid sequence with at least 90% sequence identity to, or having no more than three or two, preferably no more than one amino acid substitution(s), deletion(s) or insertion(s) compared to, a sequence selected from SEQ ID Nos.: 403, 407, 413, 417, 423, 427, 433, 437, 443, 447, 483, 487, 493, 497, 513, 517, 523, 527, 533, 537, 563, 567, 593, 597, 603, 607, 613 and 617.
   Item A12. The isolated ABP of any one of items A8 to A11, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise CDR1 to CDR3 sequences in a combination selected from any of the following combinations of heavy and/or light chain CDRs: CDRs-C-002, CDRs-C-003, CDRs-C-004, CDRs-C-005, CDRs-C-006, CDRs-C-010, CDRs-C-011, CDRs-C-013, CDRs-C-014, CDRs-C-015, CDRs-C-018, CDRs-C-021, CDRs-C-022 and CDRs-C-023,

| | Heavy Chain | | | Light Chain | | |
|---|---|---|---|---|---|---|
| Combination | CDR1 to CDR3 | | | CDR1 to CDR3 | | |
| (ID) | (SEQ ID NO) | | | (SEQ ID NO) | | |
| CDRs-C-002 | 401 | 402 | 403 | 405 | 406 | 407 |
| CDRs-C-003 | 411 | 412 | 413 | 415 | 416 | 417 |
| CDRs-C-004 | 421 | 422 | 423 | 425 | 426 | 427 |
| CDRs-C-005 | 431 | 432 | 433 | 435 | 436 | 437 |
| CDRs-C-006 | 441 | 442 | 443 | 445 | 446 | 447 |
| CDRs-C-010 | 481 | 482 | 483 | 485 | 486 | 487 |
| CDRs-C-011 | 491 | 492 | 493 | 495 | 496 | 497 |
| CDRs-C-013 | 511 | 512 | 513 | 515 | 516 | 517 |
| CDRs-C-014 | 521 | 522 | 523 | 525 | 526 | 527 |
| CDRs-C-015 | 531 | 532 | 533 | 535 | 536 | 537 |
| CDRs-C-018 | 561 | 562 | 563 | 565 | 566 | 567 |
| CDRs-C-021 | 591 | 592 | 593 | 595 | 596 | 597 |
| CDRs-C-022 | 601 | 602 | 603 | 605 | 606 | 607 |
| CDRs-C-023 | 611 | 612 | 613 | 615 | 616 | 617 |

in each case independently, optionally with no more than three or two, preferably no more than one, amino acid substitution(s), insertion(s) or deletion(s) compared to these sequences.
   Item A13. The isolated ABP of any one of items A8 to A12, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences each comprises heavy chain CDR1 to CDR3 sequences in the combination CDRs-C-003 or CDRs-C-004, or in the combination CDRs-C-005, and at least one, preferably both, of the antibody light chain sequences each comprises light chain CDR1 to CDR3 sequences in the combination, respectively, CDRs-C-003 or CDRs-C-004, or in the combination CDRs-C-005, in each case independently, optionally with no more than one amino acid substitution(s), insertion(s) or deletion(s) compared to these sequences, and preferably wherein the ABP is able to inhibit the binding of the interacting protein to IGSF11 protein or to the IgC2 domain of IGSF11 protein or, in either case, a variant thereof, with an IC50 of 50 nM or 10 nM or less.
   Item A14. An isolated ABP which competes with an ABP as recited in any one of items A8 to A13 for binding to an IgC2 domain of IGSF11 protein or a variant thereof, and, optionally, is able to inhibit the binding of an interacting protein to IGSF11 protein or to an IgC2 domain of IGSF11 protein or, in each case, a variant thereof, with the proviso that the isolated ABP is not one or more of:
any ABP the subject of proviso (A) of item A8;
any ABP the subject of proviso (B) of item A8;
any ABP the subject of proviso (C) of item A9; and/or
any ABP the subject of proviso (F) of item A10.
   Item A15. The isolated ABP of any one of items A8 to A14, wherein the interacting protein is VSIR (VISTA) protein or a variant thereof.
   Item A16. The isolated ABP of any one of items A8 to A15 that:
enhances killing and/or lysis of cells expressing IGSF11, or a variant of IGSF11, by cytotoxic T cells and/or TILs; and/or
(i) enhances a cell-mediated immune response, such as that mediated by an activated cytotoxic T-cell (CTL), to a mammalian cell expressing said IGSF11 or the variant of IGSF11; and/or (ii) increases immune cell, such as T-cell, activity and/or survival in the presence of a mammalian cell expressing said IGSF11 or the variant of IGSF11; and/or
modifies the microenvironment of a tumour, in particular modulates the number and/or type of immune cells present in the tumour, and more suitably reduces the number of intra-tumoural myeloid-derived suppressor cells (MDSCs) and/or increases the number of intra-tumoural CTLs.
   Item A17. The isolated ABP of any one of items A8 to A16 that is an antibody or an antigen binding fragment thereof, wherein the antibody is:
a monoclonal antibody, or wherein the antigen binding fragment is a fragment of a monoclonal antibody; and/or
a human antibody a humanised antibody or a chimeric-human antibody, or wherein the antigen binding fragment is a fragment of a human antibody a humanised antibody or a chimeric-human antibody.
   Item A18. A product for use in medicine, wherein the product is selected from the list consisting of:
(i) an ABP recited in any one of items A8 to A17; or
(ii) a nucleic acid encoding for an ABP, or for an antigen binding fragment or a monomer of an ABP, wherein the ABP is one recited in any one of items A8 to A17.
   Item A19. The product for use of item A18, wherein the product is for use in:
the treatment of a proliferative disorder that is associated with the undesired presence of IGSF11-positive cells or cells positive for a variant of IGSF11 and/or that is associated with cellular resistance against a cell-mediated immune response and/or that is associated with expression or activity of IGSF11 or a variant thereof of IGSF11, and optionally wherein cells involved in the proliferative disorder are resistant to a cell-mediated immune response; and or
enhancing an immune response in a mammalian subject, preferably for use in aiding a cell-mediated immune response in the subject such as the subject's T cell mediated immune response, for example for treating a proliferative disease, such as a cancer disease, or for treating an infectious disease.
   Item A20. The product for use of item A18 or A19, wherein the product is for use in the treatment of a proliferative disorder resistant and/or refractory to PD1/PDL1 blockade therapy and/or to CTLA4 blockade therapy.

Certain aspects and embodiments of the invention will now be illustrated by way of example and with reference to the description, figures and tables set out herein. Such examples of the methods, uses and other aspects of the present invention are representative only, and should not be taken to limit the scope of the present invention to only such representative examples.

The examples show:

### COMPARATIVE EXAMPLE 1: IGSF11 (VSIG3) KNOCKDOWN SENSITISES TUMOUR CELLS TOWARDS TIL-MEDIATED CYTOTOXICITY

Knockdown of IGFS11 (VSIG3) expression and hence function/activity by inhibitory nucleic acids causes a sensitisation of tumour cells to a cell-mediated immune response, even in a lung cancer cell that remains insensitive to tumour infiltrating lymphocytes (TIL)-mediated cytotoxicity despite knockdown of PD-L1. In the presence of TILs, the viability of lung cancer cells is significantly (P=0.0008) reduced for those cells treated with IGSF11 (VSIG3) siRNA (siGENOME, SMARTpool siRNA, Dharmacon, GE Healthcare) compared to those treated with negative control (Ctrl) siRNAs; and even treatment with PD-L1 siRNA shows no decrease in cell viability in the presence of TILs (FIG. 2A; CEACAM-6 siRNA as positive control), even though these cells express PD-L1 (data not shown). This observed reduction in viability was not observed in comparable experiments without TILs (FIG. 2B), and hence sensitivity of the lung cancer cells to the cytotoxic effects of TILs is therefore significantly increased and/or enhanced by the knockdown of IGSF11 (VSIG3).

Cells from the H23 non-small cell lung cancer (NSCLC) cell line (acquired from DSMZ, Germany) were stably transfected with pEGFP-luc plasmid as described in Khandelwal et al, 2015 (EMBO Mol Med 7:450). Approximately 2,000 tumour cells per well were reverse transfected with the described siRNA type (25 nM) using RNAiMAX transfection reagent (Thermo Fisher Scientific) as described before by Khandelwal et al (2015). 72 hours after siRNA transfection, the cells were co-cultured with medium alone or approximately 10,000 TILs (derived from a lung adenocarcinoma patient) for an additional 18 hours. The remaining luciferase activity associated with live tumour cells was then read out using a Tecan Spark 20M luminescence reader.

The mRNA expression of IGSF11 (VSIG3) was investigated using qPCR across a number of generally available cell lines, including those from lung (eg DMS 273 and A549), and melanoma (eg WM938B, SK-MEL-28 and M579). FIG. 7A shows that IGSF11 is relatively highly expressed by the lung cancer cell line DMS 273 and by the melanoma cell lines WM938B, SK-MEL-28 and M579-A2, while expressed at a lower level by the lung cancer cell line A549. Indeed, IGSF11 expression is noted across several tumour types in the TCGA pan-cancer genome expression database as shown by RNA expression (by RNA deep-sequencing) (FIG. 7B; figure/data from the TCGA pan-cancer genome expression database, and analysed using cBioportal for Cancer Genomics: Gao et al 2013, Sci Signal 6:pl1: Cerami et al 2012, Cancer Discov 2:401). Notably, glioblastoma (GBM), low grade glioma (LGG), uveal melanoma, melanoma, lung cancer, ovarian, pancreatic cancer etc, all show high expression of IGSF11. IGSF11 (VSIG3) is confirmed to be expressed in the H23 cells at the mRNA level (eg, by qPCR).

At least two non-overlapping IGSF11-specific siRNAs (or the siRNA pool) are tested to confirm they induced an efficient IGSF11 (VSIG3) knockdown at mRNA level as compared to scrambled control siRNA (with data normalised to eg GAPDH). Indeed, using the melanoma cell line M579-A2 (expressed high levels of IGSF11), three of the four individual siRNAs were shown to significantly reduce mRNA expression of IGSF11 by this melanoma cell line (FIG. 8A). A similar effect was also seen using the lung cancer cell line A549 (FIG. 8B), although in this case the mRNA levels measured were close to the detection limit of the assay.

Western blot and/or flow cytometry (FC) analysis can further confirm knockdown of IGSF11 (VSIG3) at the protein level by the same IGSF11-specific individual and pool siRNAs, using anti-IGSF11 antibody (eg, as described in the further examples; or anti-IGSF11 sheep polyclonal Ab cat #: AF4915 R&D Systems) and a labelled secondary antibody (eg, respectively, APC-labelled anti-human IgG, or APC-labelled anti-sheep IgG cat #: F0127 R&D Systems). The expression level of PD-L1 and CEACAM-6 in H23 cells can analogously be confirmed at the mRNA and/or protein level after treatment with gene-specific siRNA or control siRNA. For example, for western blot analysis CEACAM6 and PD-L1 can be detected with the following antibodies: anti-CEACAM6 (Abcam, Cat No: ab98109) with anti-rabbit IgG-HRP as secondary antibody (Abcam, ab97051); anti-PD-Li (R&D system; Cat. N. 130021), with secondary antibody: anti-mouse IgG-HRP (Abcam, ab6789). siRNAs which can be used to knock down IGSF11 (VSIG3), PD-L1 and/or CEACAM-6 in H23 cells, and the control siRNA are shown in Table A.

The sensitisation of H23 cell line to TIL-mediated cytotoxicity upon inhibition of IGSF11 (VSIG3) expression and/or function/activity (eg by treatment with IGSF11-specific siRNAs) can be confirmed using other assays. For example, such data are generated from: (i) classical chromium release assays conducted to directly measure specific lysis of H23 cells after co-culture with (eg, patient derived) TILs, using an assay as described by Khandelwal et al (2015); or (ii) real-time live-cell microscopy (Incucyte Zoom—Essen Bioscience) for the evaluation of tumour cell death using YOYO-1 dye, and measuring (eg, after 72h of culture) the area of YOYO-1+ cells/well as a measure of cell death. In particular, the chromium release assay can be used to investigate IGSF11-mediated sensitivity to cell-based immune responses over a range of effector (E) cell to tumour (T) cell ratios, which can confirm that eg infiltrating lymphocytes show weak cytotoxic activity against tumour cells, even at high E:T ratios, when co-cultured in the absence of IGSF11 (VSIG3) knockdown. However, IGSF11 (VSIG3) down-regulation (eg, inhibition of expression and/or activity/function) can dramatically increase TIL-mediated killing of tumour cells. Such data can confirm that the increase in T cell-mediated cytotoxicity, which is dependent on on-target gene silencing of IGSF11 (VSIG3), is observed across a wide range of E:T ratios.

Alternatively, IGSF11 (VSIG3) can be knocked-down using CRISPR/CAS9 technology, briefly as follows: IGSF11-specific guide RNAs (gRNAs) are designed using the online algorithm developed by the Broad Institute (https://portals.broadinstitute.org/gpp/public/analysis-tools/sgrna-design). Approximately 50,000 tumour cells (M579-luc, A549-luc, MCF7-luc, H23-luc etc, as applicable) are reverse transfected with the 10 nM ribonucleoprotein (RNP) mix, containing purified gRNA complexed with Cas9 protein (GeneArt Platinum, Invitrogen, Thermo), using the Lipofectamine RNAiMax transfection reagent (Thermo Fisher) in a 96-well plate. Non-targeting gRNAs and luc-specific gRNAs are used as negative and positive controls, respectively. Cells are incubated for 2 days at 37° C. followed by co-incubation with specific T cells (at 10:1 or 5:1 E:T ratio) for an additional 18 hours. Knockdown of IGSF11 expression can be confirmed at the mRNA or protein level as described above and tumour lysis induced by T cells can be measured using one or more of the assays described above, including the Luc-CTL assay as described earlier (Khandelwal et al., 2015 EMBO Mol Med).

### COMPARATIVE EXAMPLE 2: IGSF11 (VSIG3) INHIBITION SENSITISES TUMOUR CELLS OF VARIOUS CANCER TYPES TO THE ANTI-TUMOUR EFFECTS OF A CELL-MEDIATED IMMUNE RESPONSE

IGSF11 (VSIG3) plays a role in the mediation of resistance to an immune response in other tumour types, and not just the lung cell line used in Example 1. Inhibition of IGSF11 (VSIG3) eg by siRNA-based knockdown of IGSF11 (VSIG3) expression and/or function/activity in one or more of the following tumour cell lines: breast (MCF-7, MDA-MB-231, BT-474), colorectal (SW480, HTC-116), pancreatic (PANC-1), ovarian (OVCAR-3), melanoma (M579-A2), lung (A549) and myeloma (KMM1), and the subsequent challenge with survivin-specific T cells, influenza peptide-specific-T cells or (eg HLA-matched) TILs, results in significantly increased tumour cell death compared to co-culture in the absence of the T cells. Such effects can be confirmed using one or more of the assay read-out approaches described in Example 1 (eg, luc-based, chromium-release or real-time live-cell microscopy), or other suitable assays. Indeed, when tested against three different CTL types (flu-specific T cells [E:T 5:1], TIL 209 [E:T 10:1] and TIL 412 [E:T 5:1]), cells of the melanoma cell line M579-A2 expressing luciferase showed increased cytotoxicity in such a luc-based assay when treated with IGSF11 siRNAs (pool and deconvoluted individual siRNAs), compared to mock transfections or transfections with scrambled control siRNA (FIG. 9A), and often greater than the same cells treated with PD-L1 siRNA as positive control. Convincingly, the ineffective siRNA (s4) that failed to downregulate the IGSF11 mRNA levels (FIG. 8A) also failed to induce significant T cell cytotoxicity against the M579 melanoma cells. Tumour-infiltrating lymphocytes (TIL) 412 and 209 microcultures were expanded from an inguinal lymph node of a melanoma patient as described in Dudley et al (2010; Clin Cancer Res 16:6122). M579-A2-luc cells were produced from M579-A2 as described in PCT/EP2017/078856. In contrast, the only slight increases in cytotoxicity (FIG. 9B) were seen for siRNAs 1 to 3 in an analogous assay testing flu-specific T cells against cells of A459-luc (E:T ration of 5:1), a luciferase-expressing lung cancer cell line A459, that expresses only low levels of IGSF11 (see FIG. 7A). The A549 human lung cancer cell line stably expressing luciferase (A549-luc) was obtained from Gentarget. Analogous to the M579-A2-luc luciferase assay described in PCT/EP2017/078856, cells were transfected with IGSF11 siRNA, pulsed with flu (influenza-specific) peptide and co-cultured with flu-specific T cells for 20h. Subsequently, residual luciferase activity was measured as a marker of tumour cell numbers.

The expression level of IGSF11 (VSIG3) found in the various tumour cell lines tested can be determined at the mRNA or protein level (eg by qPCR or western blot as described in Example 1), and IGSF11-expression correlated to the sensitivity of each tumour cell line to TIL-mediated cytotoxicity upon treatment with IGSF11-specific siRNA. It can be shown that cell lines that do not express IGSF11 (VSIG3) do not show a decrease in viability (eg, an increase in cytotoxicity/lysis) when co-cultured with TILs after treatment with IGSF11-specific siRNA. In contrast, it can be shown that cells that do express IGSF11 (VSIG3) are—typically—more sensitive to TIL-mediated cytotoxicity after treatment with IGSF11-specific siRNA.

### EXAMPLE A: ANTI-TUMOUR PROPERTIES OF ANTI-IGSF11 ABPS DESCRIBED HEREIN

The inventors show that ABPs described herein (eg, those of Example 13) are surprisingly able to inhibit the growth of tumour in-vivo as a single agent.

ABP D-214 or D-222, in mouse IgG2a format, is evaluated for inhibiting the tumour growth of B16-F10 (C57BL6/N), Clone M3 (DBA/2N), Hepa1-6 (C57BL6/N), MC38 wt (C57BL6/N), and RENCA (BALB/c) cells implanted into the mammary fat pad of the respective strain of female mice (strain as specified in parenthesis). The study consists of 5 different tumour models. Each tumour model includes 4 experimental groups, each containing 10 female mice after randomisation.

On Day 0, tumour cells (0.2×10e6 B16-F10 cells/1.0×10e6 Clone M3 cells/2.0×10e6 Hepa1-6/1.0×10e6 MC38 wt cells/1.0×10e6 RENCA cells all in 100 ul PBS) are implanted into the mammary fat pad of each mouse. Primary tumour volumes are determined by calliper measurement. Tumour sizes are calculated according to the formula W2×L/2 (L=length and W=the perpendicular width of the tumour, L>W). Animals are allocated in treatment groups with an average tumour volume between 100-150 mm3, and treatments initiated on the same day. Test compound D-214 or D-222 and anti-PD-1 mAb (clone: RMP1-14, BioXcell) or their corresponding controls mIgG2a_ctrl. or ratIgG2a_ctrl. (clone: 2A3, BioXcell) are administered two times weekly at 15 mg/kg or 10 mg/kg respectively according to Table A.4 starting at the day of group assignment.

TABLE A.4
Treatment groups for tumour growth kinetics in B16-F10, Clone M3,
Hepa1-6, MC38wt and RENCA

| | | | Dose | | | | Number |
|---|---|---|---|---|---|---|---|
| | | Dose | Volume | Administration | Dosing | | of |
| Group | Test specimen | (mg/kg)* | (ml/kg) | route** | frequency | Vehicle | animals |
| 1 | anti-ratIgG2a-ctrl + | 10.0 + 15.0 | 10.0 + 10.0 | i.p. + i.p. | 2 times per | PBS | 10 |
| | anti-mIgG2a-ctrl + | | | | week | | |
| 2 | anti-mPD-1 | 10.0 + 15.0 | 10.0 + 10.0 | i.p. + i.p. | 2 times per | PBS | 10 |
| | Anti-mIgG2a-ctrl | | | | week | | |
| 3 | anti-ratIgG2a-ctrl + | 10.0 + 15.0 | 10.0 + 10.0 | i.p. + i.p. | 2 times per | PBS | 10 |
| | D-214 or D-222 | | | | week | | |
| 4 | anti-mPD-1 + | 10.0 + 15.0 | 10.0 + 10.0 | i.p. + i.p. | 2 times per | PBS | 10 |
| | D-214 or D-222 | | | | week | | |

*Based on last bodyweight measurement;
**i.p. = intraperitoneal

Individual animals are euthanised due to ethical abortion criteria prior study end without any necropsy. A final necropsy of the animals of all groups is performed latest when the 4th mouse of any group needs to be euthanised due to ethical abortion criteria. At final necropsy, animals are weighed, and in vivo tumour volume measurement is performed. A final bleeding is performed from the 6 animals of which the tumour is used for flow cytometry analysis. Finally, all animals are euthanised by cervical dislocation.

Primary tumour tissues are collected, and wet weight and tumour volume determined. Selected tumours (6 tumours of Group 1-4) are prepared for flow cytometry analysis. The selection of tumours for flow cytometry represents the overall distribution of tumour sizes in each treatment group. Tumour tissues of tumours not selected for flow cytometry are snap-frozen in liquid nitrogen, transferred to polypropylene tubes and stored appropriately at −80° C.

At necropsy, animals of which the tumour is used for flow cytometry analysis are anaesthetised by isoflurane and blood taken with micro capillaries via retro-orbital vein puncture (terminal blood sampling) slightly rotated, and immediately transferred into EDTA coated tubes (K2E tubes) on ice. To obtained EDTA-plasma, tubes are centrifuged at 4° C. for 10 min at 8000 rpm (6800 g). After centrifugation, the supernatant is transferred to a new polypropylene tube labelled and stored at −80° C. EDTA plasma samples are used for drug level determination via ELISA or Bio-Layer Interferometry.

Tumours for flow cytometry analysis are collected and processed for analysis, after primary tumour wet weights and volumes have been determined. Primary tumour material (approx. 200-300 mg) is disrupted using gentleMACS™ C Tubes containing the enzyme mix of the Tumor Dissociation Kit according to the manufacturer instructions (Miltenyi Biotec, Germany). Erythrocytes are removed with the Red Blood Cell Lysis Solution (Miltenyi Biotec, Germany). Obtained single cell suspensions from tumour are counted and dispensed in 96 well plates.

Staining A: Single cells are washed with PBS and stained for living cells for 30 min (FVS780, Becton Dickinson). After washing and centrifugation (400 g) the samples are incubated with 50 ul/well Fc block (anti-Mouse CD16/CD32, 1:50) for 15 min in FACS buffer. Thereafter, a 2× concentrated master antibody mix (Table A.5 CD3, CD4, CD8a, CD45, CD25, CD11b, Ly6C, Ly6G, F4/80, CD11c, MHC class II, CD206, CD335, CD49b, B220) is added to each well (50 ul) and incubated for 30 min in the dark. After washing, intracellular staining is primed by adding 100 ul fix/perm buffer (one-part fixation/permeabilisation concentrate to three parts fixation/permeabilisation diluent) for 30 min. After centrifugation at 840 g, the cell pellet is resuspended in 1× permeabilisation buffer containing the anti-FoxP3 antibody and incubated for 30 min in the dark. After washing with 1× permeabilisation buffer cells are washed with FACS buffer. The cells are resuspended in FACS buffer and kept at 4° C. in the dark until analysis no later than 5 days after preparation. The samples will be analysed by flow cytometry using a LSR Fortessa (Becton Dickinson).

TABLE A.5
Myeloid and lymphoid immune cell panel

| Antibody | Color | Catalogue # |
|---|---|---|
| Fixable Viability Stain 780 | | 565388 |
| Hamster Anti-Mouse CD3e | BV786 | 564379 |
| Rat Anti-Mouse CD4 | BUV496 | 564667 |
| Rat Anti-Mouse CD8a | APC-R700 | 564983 |
| Rat Anti-Mouse CD45 | BUV395 | 565967 |
| Rat Anti-Mouse CD25 | BB515 | 564424 |
| Rat Anti-CD11b | BV711 | 563168 |
| Rat Anti-Mouse Ly-6C | PE-Cy ™ 7 | 560593 |
| Rat Anti-Mouse LY-6G | BV605 | 563005 |
| Rat Anti-Mouse F4/80 | PE-CF594 | 565613 |
| Hamster Anti-Mouse CD11c | PE | 557401 |
| Rat Anti-Mouse I-A/I-E | PerCP-Cy ™ 5.5 | 562363 |
| Rat Anti-Mouse CD206 | Alexa Fluor ® 647 | 565250 |
| Rat Anti-Mouse CD335 | BV650 | 740627 |
| (NKp46) | | |
| Hamster Anti-Mouse CD49b | BV510 | 740133 |
| Rat Anti-Mouse CD45R/B220 | BV421 | 562922 |
| anti-mouse Foxp3 | PE | 12-5773-80 |

Staining B: Single cells are stimulated in 200 ul with PMA (5 ng/ml)/Ionomycin (500 ng/ml)/Golgiplug for 4 hours in complete RPMI medium (10% FCS, β-mercaptoethanol (55 uM, 1:260.000 dilution of a 14.3M solution)) at 37° C. Thereafter stimulated cells are washed twice with PBS and stained for living cells for 15 min (Zombie Aqua™ Fixable Viability Kit, cat #423102, Biolegend). After washing in FACS buffer and centrifugation (400 g) the samples are incubated with 50 ul/well Fc block (anti-Mouse CD16/CD32, 1:20) for 10 min in FACS buffer. Thereafter, a 2× concentrated master antibody mix (Table A.6: CD3e, CD69, CD45, CD11b, CD4, CD8, CD25, CD107a) is added to each well (50 ul) and incubated for 30 min in the dark on ice. After washing, intracellular staining is primed by adding 100 ul fix/perm buffer (one-part fixation/permeabilisation concentrate to three parts fixation/permeabilisation diluent) for 30 min. After centrifugation at 840 g, the cell pellet will be resuspended in 1× permeabilisation buffer containing a master antibody mix directed against IFN-gamma, Granzyme B, and FoxP3 and incubated for 30 min in the dark. After washing with 1× permeabilisation buffer cells are washed with FACS buffer. The cells are resuspended in FACS buffer and kept at 4° C. in the dark until analysis no later than 5 days after preparation. The samples are analysed by flow cytometry using a LSR Fortessa (Becton Dickinson).

Statistical analysis of flow cytometry analysis is performed by One-way ANOVA using Tukeys multiple comparisons test. Statistical analysis of tumour growth kinetic data is performed by Two-way ANOVA using Sidaks multiple comparisons test.

TABLE A.6
Activated T cell panel

| Antibody | Color | Catalogue # |
|---|---|---|
| Live/dead | AquaZombie | 423102 |
| CD3e | Alexa 488 | 557666 |
| CD69 | PerCp-Cy5.5 | 104522 |
| CD45 | AF700 | 103128 |
| CD11b | APC Cy7 | 557657 |
| CD4 | eF450 | 48-0042-82 |
| CD8 | BV650 | 100741 |
| CD25 | PE | 553866 |
| CD107a | PE-CY7 | 121620 |
| IFNg | BV605 | 505840 |
| GranzymeB | PE-CF594 | 562462 |
| FoxP3 | APC | 17-5773-82 |

### EXAMPLE B: EXPRESSION OF IGSF11 ON IMMUNE AND TUMOUR CELLS

The inventors show that although VISTA expression is predominantly detected on myeloid cells in peripheral blood, no VISTA expression is observed on in vitro differentiated macrophages. Following subsequent investigation IGSF11 expression is not (typically and/or reliably) detected on healthy human donor PBMCs or in-vitro differentiated macrophages (FIG. 25). Expression of IGSF11 was detected on various immune cells essentially as described in Comparative Example 6. VISTA expression on such cells was detected analogously but using of an anti-VISTA primary antibody.

Using an improved FISH technology (RNAscope; Advanced cell diagnostics) on tissue microarrays, expression of IGSF11 was shown exclusively on tumour cells (FIGS. 26A and B) and not on infiltrating stroma (FIG. 26C) or corresponding healthy tissue (data not shown). In particular, IGSF11 was shown to be overexpressed on cells from solid tumour such as lung, melanoma, head and neck squamous cell carcinoma (HNSCC), bladder, thymoma and ovarian cancer. Expression of IGSF11 in healthy tissue was restricted to immune-privileged organs such as cerebellum, testis, and ovary tissue (data not shown).

### EXAMPLE C: EXPRESSION OF IGSF11 IN CANCER PATIENTS TREATED IN CLINICAL TRIALS WITH ANTI-PD1 CHECKPOINT INHIBITORS

Using data published by Riaz et al (2017, Cell 171:934), IGSF11 expression in 33 melanoma patients treated with the anti-PD1 inhibitor nivolumab (OPDIVO) was analysed. The results of such analysis demonstrate that base-line expression of IGSF11 was increased in non-responding patients (progressive disease or stable disease) compared to those who responded (complete or partial response) to nivolumab treatment (FIG. 27A). Indeed, upon treatment with nivolumab the difference in IGSF11 expression between these responding and non-responding patients was highly increased (FIG. 27B).

Analogous analysis of IGSF11 expression in 144 melanoma patients treated with either nivolumab or another anti-PD1 inhibitor (pembrolizumab; KEYTRUDA) published by Liu et al (2019, Nat Med 25:1916), or in 35 clear cell renal cell carcinoma (ccRCC) patients treated with nivolumab published by Miao et al (2018, Science 359:801) provides further evidence and supports the finding that IGSF11 expression is higher in those patients who do not respond to anti-PD1 treatment, compared to those who respond to anti-PD1 treatment (data not shown).

Such evidence supports the treatment of cancer patients with IGSF11 modulators, such as anti-IGSF11 ABPs of the invention, in combination with anti-PD1 checkpoint inhibitors, or the treatment of cancer patients who have not responded to anti-PD1 treatment with IGSF11 modulators, such as anti-IGSF11 ABPs of the invention, as sole agent.

Furthermore, a negative correlation of IGSF expression with a measure of tumour inflammation was demonstrated (eg, FIG. 27C for data from Riaz et al 2017).

Gene expression datasets from melanoma (Riaz et al 2017; Liu et al 2019) and renal cell carcinoma (Miao et al 2018) patients treated with nivolumab or pembrolizumab were downloaded as fastq from Sequence Read Archive (SRA). Reads for each sample were aligned using HISAT2 and gene counts were calculated by StringTie. Response categories (PD: progressive disease, SD: stable disease, CR/PR: complete response and partial response or CR/PR/MR: complete response, partial response and mixed response) were collected from clinical data associated with the expression data. A multi-gene marker for immune cell infiltration and activity was calculated for each tumour sample similarly as described by Damotte et al (2019, J Trans Med 17:357) and correlated with IGSF11 expression using the ggscatter package of R.

### Example D: Receptor Internalisation by ABPs of the Invention

The inventors investigate internalisation of surface-expressed IGSF11 protein on tumour cells by ABPs of the invention that bind to the IgV domain of IGSF11 (eg, C-001) and (eg, compared to) those that bind to the IgC2 domain of IGSF11 (eg D-214 or D-222.

The internalisation assay is performed as follows. Briefly, endogenous IGFS11 expressing Colo741 cells are seeded at 100,000 cells/well into a 96 well plate. FC blocking is performed using ChromPure human IgG blocking solution for 30 min on ice. Cells are washed once and 0.5 ug/ml APBs of the invention are added to respective wells. Cells are incubated for 30, 60, 120, and 240 min both at 4° C. (control sample) and 37° C. (internalisation sample). After the respective incubation time, cells are washed twice and labelled with 1.25 ug/ml secondary anti-human IgG F(ab′)2 antibody for 30 min on ice in the dark. Cells are again washed twice and resuspended in diluted 7-AAD immediately before acquisition on the iQue flow cytometer. % Internalisation is calculated using the following formula: % Internalisation=100−((Mean internalisation sample/Mean control sample)*100).

### COMPARATIVE EXAMPLE 3: GENERATION OF ANTIBODIES THAT BIND TO HUMAN IGSF11 (VSIG3)

Human scFv antibodies that bind human IGSF11 (VSIG3) were identified. Two universal human scFv antibody-phage libraries (Yumab GmbH, Braunschweig, Germany), consisting either solely of human kappa or lambda antibodies and comprising a diversity of more than 1×10e10 different antibody sequences, were screened for binding to the extracellular domain (ECD) of human IGSF11 (VSIG3). Conventional phage-display and panning protocols were used by Yumab in different selection strategies, each strategy comprising three rounds of selection using different variants of the biotinylated ECDs of human or murine IGSF11 protein and/or transfected HEK cells expressing human IGSF11 protein. Panning-derived hits were further selected for preferential binding to the (streptavidin-captured) positive antigens, human and mouse IGSF11, compared to a negative antigen of streptavidin and/or murine-Fc domain. Binding of certain of such hits to cynomolgus monkey IGSF11 can also be tested.

scFv antibodies of Comparative Example 3 that selectively bind the ECD of human and murine IGSF11 protein over streptavidin and murine-Fc domain are identified and described in Tables 1, showing for each such antibody the heavy chain and light chain CDR sequences and variable region sequences comprised in each such antibody as well as nucleic acid sequences encoding for such variable regions (Table 1A), and the identification of the human germ-line genes for the variable regions (Table 1B and/or Table 1B.1). The degree of binding of each such antibody to human and murine IGSF11 protein (and to irrelevant antigen), as determined by ELISA, and to human IGSF11 protein expressed by cells, as determined by flow cytometry (FC), is shown in Table 2.

TABLE 1A
Amino acid sequences of CDR and variable regions of ABPs of Comparative Example 3, as well
as nucleic acid sequences encoding variable regions of ABPs of Comparative Example 3.

| Anti- | | Sequence | SEQ |
|---|---|---|---|
| body | Region | 1.......10.......20.......30.......40.......50 | ID NO. |
| A-001 | H-CDR1 | SYSMN | 1 |
| | H-CDR2 | SISSSSSYIYYADSVKG | 2 |
| | H-CDR3 | SIIVQALGITSVFDI | 3 |
| | VH (aa) | EVQLVESGGGLVKPGGSLRLSCAASGFTFSSYSMNWVRQAPGKGLEWVSSI | 4 |
| | | SSSSSYIYYADSVKGRFTISRDNAKNSLYLQMNSLRAEDTAVYYCVRSIIV | |
| | | QALGITSVFDIWGQGTMVTVSS | |
| | L-CDR1 | RASQIISNHLN | 5 |
| | L-CDR2 | AASRLQT | 6 |
| | L-CDR3 | QQSYSNPRT | 7 |
| | VL (aa) | AIRLTQSPSSLSASVGDRVTISCRASQIISNHLNWYQQKPGKAPKLLIYAA | 8 |
| | | SRLQTGVPSRFSGSGSETDYTLTISSLQPEDFATYYCQQSYSNPRTFGHGT | |
| | | KVEIK | |
| | VH | GAGGTGCAGCTGGTGGAGTCTGGGGGAGGCCTGGTCAAGCCTGGGGGGTCC | 9 |
| | (DNA) | CTGAGACTCTCCTGTGCAGCCTCTGGATTCACCTTCAGTAGCTATAGCATG | |
| | | AACTGGGTCCGCCAGGCTCCAGGGAAGGGGCTGGAGTGGGTCTCATCCATT | |
| | | AGTAGTAGTAGTAGTTACATATACTACGCAGACTCAGTGAAGGGCCGATTC | |
| | | ACCATCTCCAGAGACAACGCCAAGAACTCACTGTATCTGCAAATGAACAGC | |
| | | CTGAGAGCCGAGGACACGGCTGTATATTACTGTGTCAGGTCTATTATAGTT | |
| | | CAAGCACTTGGCATAACGTCCGTTTTTGATATCTGGGGCCAAGGGACAATG | |
| | | GTCACCGTCTCTTCA | |
| | VL | GCCATCCGGTTGACCCAGTCTCCATCCTCCCTGTCTGCATCTGTAGGAGAC | 10 |
| | (DNA) | AGAGTCACCATCTCTTGCCGGGCAAGTCAGATCATTAGCAATCATTTAAAT | |
| | | TGGTATCAGCAGAAACCAGGAAAAGCCCCTAAGCTCCTGATCTATGCTGCA | |
| | | TCCAGATTGCAAACTGGGGTCCCATCAAGGTTCAGTGGCAGTGGCTCTGAG | |
| | | ACAGACTACACTCTCACCATCAGCAGTCTGCAACCTGAAGATTTTGCAACT | |
| | | TACTACTGTCAACAGAGTTACAGTAACCCCCGGACGTTCGGCCACGGGACC | |
| | | AAGGTGGAAATCAAA | |
| A-002 | H-CDR1 | SNYMS | 11 |
| | H-CDR2 | VIYSGGSTYYADSVKG | 12 |
| | H-CDR3 | GNPYYYGDLQVNFDY | 13 |
| | VH (aa) | EVQLVESGGGLIQPGGSLRLSCAASGFTVSSNYMSWVRQAPGKGLEWVSVI | 14 |
| | | YSGGSTYYADSVKGRFTISRDNSKNTLYLQMNSLRAEDTAVYYCARGNPYY | |
| | | YGDLQVNFDYWGQGTLVTVSS | |
| | L-CDR1 | GSSTGAVTSGNLPN | 15 |
| | L-CDR2 | STSNKHS | 16 |
| | L-CDR3 | LLYYGGAWV | 17 |
| | VL (aa) | QAVVTQEPSLTVSPGGTVTPTCGSSTGAVTSGNLPNWFQQKPGQAPRALIY | 18 |
| | | STSNKHSWTPARISGSLLGGKAALTLSGVQPEDEAEYYCLLYYGGAWVFGG | |
| | | GTKLTVL | |
| | VH | GAAGTGCAGCTGGTGGAGTCTGGAGGAGGCTTGATCCAGCCTGGGGGGTCC | 19 |
| | (DNA) | CTGAGACTCTCCTGTGCAGCCTCTGGGTTCACCGTCAGTAGCAACTACATG | |
| | | AGCTGGGTCCGCCAGGCTCCAGGGAAGGGGCTGGAGTGGGTCTCAGTTATT | |
| | | TATAGCGGTGGTAGCACATACTACGCAGACTCCGTGAAGGGCCGATTCACC | |
| | | ATCTCCAGAGACAATTCCAAGAACACGCTGTATCTTCAAATGAACAGCCTG | |
| | | AGAGCCGAGGACACGGCCGTGTATTACTGTGCGAGAGGTAACCCATATTAC | |
| | | TACGGTGACCTCCAGGTGAACTTTGACTACTGGGGCCAGGGAACCCTGGTC | |
| | | ACCGTCTCCTCA | |
| | VL | CAGGCTGTGGTGACTCAGGAGCCCTCACTGACTGTGTCCCCAGGAGGGACA | 20 |
| | (DNA) | GTCACTCCCACCTGTGGTTCCAGCACTGGAGCAGTCACCAGTGGTAACCTT | |
| | | CCAAACTGGTTCCAGCAGAAACCTGGACAAGCACCCAGGGCACTGATTTAT | |
| | | AGTACAAGCAACAAACACTCCTGGACCCCTGCCCGGATCTCAGGCTCCCTC | |
| | | CTTGGGGGCAAAGCTGCCCTGACACTGTCAGGTGTGCAGCCTGAGGACGAG | |
| | | GCTGAATATTACTGTCTACTCTATTATGGTGGTGCTTGGGTGTTCGGCGGA | |
| | | GGGACCAAGCTGACCGTCCTA | |
| A-003 | H-CDR1 | SYAIS | 21 |
| | H-CDR2 | GIIPIFGTANYAQKFQG | 22 |
| | H-CDR3 | PRIQLWAAGGFDY | 23 |
| | VH (aa) | VQLVQSGAEVKKPGSSVKVSCKASGGTFSSYAISWVRQAPGQGLEWMGGII | 24 |
| | | PIFGTANYAQKFQGRVTITADESTSTAYMELSSLRSEDTAVYYCASPRIQL | |
| | | WAAGGFDYWGQGTLVTVSS | |
| | L-CDR1 | TGTSSDVGGYNLVS | 25 |
| | L-CDR2 | DVSNRPS | 26 |
| | L-CDR3 | SSFTTSTTLV | 27 |
| | VL (aa) | QSALTQPASVSGSPGQSITISCTGTSSDVGGYNLVSWYQQHPGKAPKLMIY | 28 |
| | | DVSNRPSGVSNRFSGSKSGNTASLTISGLQAEDEADYYCSSFTTSTTLVFG | |
| | | GGTKLTVL | |
| | VH | GTGCAGCTGGTGCAGTCTGGGGCTGAGGTGAAGAAGCCTGGGTCCTCGGTG | 29 |
| | (DNA) | AAGGTCTCCTGCAAGGCTTCTGGAGGCACCTTCAGCAGCTATGCTATCAGC | |
| | | TGGGTGCGACAGGCCCCTGGACAAGGGCTTGAGTGGATGGGAGGGATCATC | |
| | | CCTATCTTTGGTACAGCAAACTACGCACAGAAGTTCCAGGGCAGAGTCACG | |
| | | ATTACCGCGGACGAATCCACGAGCACAGCCTACATGGAGCTGAGCAGCCTG | |
| | | AGATCTGAGGACACGGCCGTGTATTACTGTGCGAGCCCCAGGATACAGCTA | |
| | | TGGGCCGCCGGGGGCTTTGACTACTGGGGCCAGGGAACCCTGGTCACTGTC | |
| | | TCCTCA | |
| | VL | CAGTCTGCCCTGACTCAGCCTGCCTCCGTGTCTGGGTCTCCTGGACAGTCG | 30 |
| | (DNA) | ATCACCATCTCCTGCACTGGAACCAGCAGTGACGTTGGTGGTTATAACCTT | |
| | | GTCTCCTGGTACCAACAGCACCCAGGCAAAGCCCCCAAACTCATGATTTAT | |
| | | GATGTCAGTAATCGGCCCTCAGGGGTTTCTAATCGCTTCTCTGGCTCCAAG | |
| | | TCTGGCAACACGGCCTCCCTGACCATCTCTGGGCTCCAGGCTGAGGACGAG | |
| | | GCTGATTATTATTGCAGCTCATTTACAACTAGCACCACTCTAGTATTCGGC | |
| | | GGAGGGACCAAACTGACCGTCCTA | |
| A-004 | H-CDR1 | SYSMN | 31 |
| | H-CDR2 | SISSSSSYIYYADSVKG | 32 |
| | H-CDR3 | GQLLWFGESALIDAFDI | 33 |
| | VH (aa) | EVQLVESGGGLVKPGGSLRLSCAASGFTFSSYSMNWVRQAPGKGLEWVSSI | 34 |
| | | SSSSSYIYYADSVKGRFTISRDNAKNSLYLQMNSLRAEDTAVYYCARGQLL | |
| | | WFGESALIDAFDIWGQGTMVTVSS | |
| | L-CDR1 | RASQSISSYLN | 35 |
| | L-CDR2 | AASILQS | 36 |
| | L-CDR3 | QQSYSTPRT | 37 |
| | VL (aa) | EIVLTQSPSSLSASVGDRVTITCRASQSISSYLNWYQLKPGKAPKLLIYAA | 38 |
| | | SILQSGVPSRFSGSGSGTDFTLTISSLQPEDFAAYYCQQSYSTPRTFGQGT | |
| | | KLEIK | |
| | VH | GAAGTGCAGCTGGTGGAGTCTGGGGGAGGCCTGGTCAAGCCTGGGGGGTCC | 39 |
| | (DNA) | CTGAGACTCTCCTGTGCAGCCTCTGGATTCACCTTCAGTAGCTATAGCATG | |
| | | AACTGGGTCCGCCAGGCTCCAGGGAAGGGGCTGGAGTGGGTCTCATCCATT | |
| | | AGTAGTAGTAGTAGTTACATATACTACGCAGACTCAGTGAAGGGCCGATTC | |
| | | ACCATCTCCAGAGACAACGCCAAGAACTCACTGTATCTGCAAATGAACAGC | |
| | | CTGAGAGCCGAGGACACGGCTGTGTATTACTGTGCGAGAGGTCAATTACTA | |
| | | TGGTTCGGGGAGTCAGCACTGATTGATGCTTTTGATATCTGGGGCCAAGGG | |
| | | ACAATGGTCACCGTCTCTTCA | |
| | VL | GAAATTGTGTTGACGCAGTCTCCATCCTCCCTGTCTGCATCTGTAGGAGAC | 40 |
| | (DNA) | AGAGTCACCATCACTTGCCGGGCAAGTCAGAGCATTAGCAGCTATTTAAAT | |
| | | TGGTATCAGCTGAAACCAGGGAAAGCCCCTAAACTCCTGATCTATGCTGCA | |
| | | TCCATTCTGCAAAGTGGGGTCCCATCAAGGTTCAGTGGCAGTGGATCTGGG | |
| | | ACAGATTTCACTCTCACCATCAGCAGTCTGCAACCTGAAGATTTTGCAGCT | |
| | | TACTACTGTCAACAGAGTTACAGTACCCCTCGGACTTTTGGCCAGGGGACC | |
| | | AAGCTGGAGATCAAA | |
| A-005 | H-CDR1 | SNYMS | 41 |
| | H-CDR2 | VIYSGGSTYYADSVKG | 42 |
| | H-CDR3 | GNPYYYGDLQVNFDY | 43 |
| | VH (aa) | EVQLVESGGGLIQPGGSLRLSCAASGFTVSSNYMSWVRQAPGKGLEWVSVI | 44 |
| | | YSGGSTYYADSVKGRFTISRDNSKNTLYLQMNSLRAEDTAVYYCARGNPYY | |
| | | YGDLQVNFDYWGQGTLVTVSS | |
| | L-CDR1 | ASSTGAVTSGYYPN | 45 |
| | L-CDR2 | STSNKDS | 46 |
| | L-CDR3 | LLYYGGAWV | 47 |
| | VL (aa) | QAVVTQEPSLTVSPGETVTLTCASSTGAVTSGYYPNWFQQRPGQAPRALIY | 48 |
| | | STSNKDSWTPARFSGSLLGGKAALTLSGVQPEDEAEYYCLLYYGGAWVFGG | |
| | | GTKLTVL | |
| | VH | GAGGTGCAGCTGGTGGAGTCTGGAGGAGGCTTGATCCAGCCTGGGGGGTCC | 49 |
| | (DNA) | CTGAGACTCTCCTGTGCAGCCTCTGGGTTCACCGTCAGTAGCAACTACATG | |
| | | AGCTGGGTCCGCCAGGCTCCAGGGAAGGGGCTGGAGTGGGTCTCAGTTATT | |
| | | TATAGCGGTGGTAGCACATACTACGCAGACTCCGTGAAGGGCCGATTCACC | |
| | | ATCTCCAGAGACAATTCCAAGAACACGCTGTATCTTCAAATGAACAGCCTG | |
| | | AGAGCCGAGGACACGGCCGTGTATTACTGTGCGAGAGGTAACCCATATTAC | |
| | | TACGGTGACCTCCAGGTGAACTTTGACTACTGGGGCCAGGGAACCCTGGTC | |
| | | ACCGTCTCCTCA | |
| | VL | CAGGCTGTGGTGACTCAGGAGCCCTCACTGACTGTGTCCCCAGGAGAGACG | 50 |
| | (DNA) | GTCACTCTCACCTGTGCTTCCAGCACTGGAGCAGTCACCAGTGGTTACTAT | |
| | | CCAAACTGGTTCCAGCAGAGACCTGGACAAGCACCCAGGGCACTGATTTAT | |
| | | AGTACAAGCAACAAAGACTCCTGGACCCCTGCCCGGTTCTCAGGCTCCCTC | |
| | | CTTGGGGGCAAAGCTGCCCTGACACTGTCAGGTGTGCAGCCTGAGGACGAG | |
| | | GCTGAGTATTACTGCCTGCTCTACTATGGTGGTGCTTGGGTGTTCGGCGGA | |
| | | GGGACCAAGCTGACCGTCCTA | |
| A-006 | H-CDR1 | SNYMS | 51 |
| | H-CDR2 | VIYSGGSTYYADSVKG | 52 |
| | H-CDR3 | GNPYYYGDLQVNFDY | 53 |
| | VH (aa) | EVQLVESGGGLIQPGGSLRLSCAASGFTVSSNYMSWVRQAPGKGLEWVSVI | 54 |
| | | YSGGSTYYADSVKGRFTISRDNSKNTLYLQMNSLRAEDTAVYYCARGNPYY | |
| | | YGDLQVNFDYWGQGTLVTVSS | |
| | L-CDR1 | ASSTGAVTSGYYPN | 55 |
| | L-CDR2 | STSNKHS | 56 |
| | L-CDR3 | LLYYGGDWV | 57 |
| | VL (aa) | QAWTQEPSLTVSPGGTVTLTCASSTGAVTSGYYPNWFQQKPGQAPRPLIY | 58 |
| | | STSNKHSWTPARFSGSLLGGKAALTLSGVQPDDEAEYYCLLYYGGDWVFGG | |
| | | GTKLTVL | |
| | VH | GAGGTGCAGCTGGTGGAGTCTGGAGGAGGCTTGATCCAGCCTGGGGGGTCC | 59 |
| | (DNA) | CTGAGACTCTCCTGTGCAGCCTCTGGGTTCACCGTCAGTAGCAACTACATG | |
| | | AGCTGGGTCCGCCAGGCTCCAGGGAAGGGGCTGGAGTGGGTCTCAGTTATT | |
| | | TATAGCGGTGGTAGCACATACTACGCAGACTCCGTGAAGGGCCGATTCACC | |
| | | ATCTCCAGAGACAATTCCAAGAACACGCTGTATCTTCAAATGAACAGCCTG | |
| | | AGAGCCGAGGACACGGCCGTGTATTACTGTGCGAGAGGTAACCCATATTAC | |
| | | TACGGTGACCTCCAGGTGAACTTTGACTACTGGGGCCAGGGAACCCTGGTC | |
| | | ACCGTCTCCTCA | |
| | VL | CAGGCTGTGGTGACTCAGGAGCCCTCACTGACTGTGTCCCCAGGAGGGACA | 60 |
| | (DNA) | GTCACTCTCACCTGTGCTTCCAGCACTGGAGCAGTCACCAGTGGTTACTAT | |
| | | CCAAACTGGTTCCAGCAGAAACCTGGACAAGCACCCAGGCCACTGATTTAT | |
| | | AGTACAAGCAACAAACACTCCTGGACCCCTGCCCGGTTCTCAGGCTCCCTC | |
| | | CTTGGGGGCAAAGCTGCCCTGACACTGTCAGGTGTGCAGCCTGACGACGAG | |
| | | GCTGAATATTACTGCCTGCTCTACTATGGTGGTGACTGGGTGTTCGGCGGA | |
| | | GGGACCAAGCTGACCGTCCTA | |
| A-007 | H-CDR1 | GYYMH | 61 |
| | H-CDR2 | WINPNSGGTNYAQKFQG | 62 |
| | H-CDR3 | GSNFDY | 63 |
| | VH (aa) | QVQLVQSGAEVKKPGASVKVSCKASGYTFTGYYMHWVRQAPGQGLEWMGW1 | 64 |
| | | NPNSGGTNYAQKFQGRVTMTRDTSISTAYMELSRLRSDDTAVYYCAAGSNF | |
| | | DYWGQGTLVTVSS | |
| | L-CDR1 | TGSSSDIGSFSYVS | 65 |
| | L-CDR2 | GVNNRPL | 66 |
| | L-CDR3 | SSYTRRSTVI | 67 |
| | VL (aa) | QSALTQPASVSGSPGQSITIACTGSSSDIGSFSYVSWYQQRPGKAPTLIIY | 68 |
| | | GVNNRPLGVSRRFSGSKSGNTASLSISGLQAEDEADYYCSSYTRRSTVIFG | |
| | | GGTKLTVL | |
| | VH | CAGGTTCAGCTGGTGCAGTCTGGGGCTGAGGTGAAGAAGCCTGGGGCCTCA | 69 |
| | (DNA) | GTGAAGGTCTCCTGCAAGGCTTCTGGATACACCTTCACCGGCTACTATATG | |
| | | CACTGGGTGCGACAGGCCCCTGGACAAGGGCTTGAGTGGATGGGATGGATC | |
| | | AACCCTAACAGTGGTGGCACAAACTATGCACAGAAGTTTCAGGGCAGGGTC | |
| | | ACCATGACCAGGGACACGTCCATCAGCACAGCCTACATGGAGCTGAGCAGG | |
| | | CTGAGATCTGACGACACGGCCGTGTATTACTGTGCTGCGGGGTCTAACTTT | |
| | | GACTACTGGGGCCAGGGAACCCTGGTCACCGTCTCCTCA | |
| | VL | CAGTCTGCCCTGACTCAGCCTGCCTCCGTGTCTGGGTCTCCGGGACAATCG | 70 |
| | (DNA) | ATCACCATCGCCTGCACTGGAAGCAGTAGTGACATTGGTAGTTTTTCTTAT | |
| | | GTCTCCTGGTACCAACAGCGCCCCGGCAAAGCCCCCACACTCATCATTTAT | |
| | | GGGGTCAATAATCGACCCTTAGGGGTGTCTCGGCGCTTCTCTGGCTCCAAG | |
| | | TCTGGCAACACGGCCTCCCTAAGCATCTCTGGGCTCCAGGCTGAGGACGAG | |
| | | GCTGATTATTACTGCAGTTCCTATACACGCAGAAGCACTGTGATCTTCGGC | |
| | | GGCGGGACCAAGTTGACCGTCCTA | |
| A-008 | H-CDR1 | SSGYYWG | 71 |
| | H-CDR2 | SIYYSGSTYYNPSLKS | 72 |
| | H-CDR3 | HRVRFGEFDAFDI | 73 |
| | VH (aa) | QLQLQESGPGLVKPSETLSLTCTVSGGSISSSGYYWGWIRQPPGKGLEWIG | 74 |
| | | SIYYSGSTYYNPSLKSRVTISVDTSKNQFSLKLSSVTAADTAVYYCARHRV | |
| | | RFGEFDAFDIWGQGTMVTVSS | |
| | L-CDR1 | TGTSSNVGADFDVH | 75 |
| | L-CDR2 | GSDNRPS | 76 |
| | L-CDR3 | QAYDVRLSGWV | 77 |
| | VL (aa) | QAVLTQPPSVSGAPGERVTLSCTGTSSNVGADFDVHWYQQFPGTAPRLLIF | 78 |
| | | GSDNRPSGVPDRFSGSKSGTSASLAITGLQVEDEADYYCQAYDVRLSGWVF | |
| | | GGGTKLTVL | |
| | VH | CAGCTGCAGCTGCAGGAGTCGGGCCCAGGACTGGTGAAGCCTTCGGAGACC | 79 |
| | (DNA) | CTGTCCCTCACCTGCACCGTCTCTGGTGGCTCCATCAGCAGTAGTGGTTAC | |
| | | TACTGGGGCTGGATCCGCCAGCCCCCAGGGAAGGGGCTGGAGTGGATTGGG | |
| | | AGTATCTATTATAGTGGGAGCACCTACTACAACCCGTCCCTCAAGAGTCGA | |
| | | GTCACCATATCCGTAGACACGTCTAAGAACCAGTTCTCCCTGAAGCTGAGC | |
| | | TCTGTGACCGCCGCAGACACGGCTGTGTATTACTGTGCGAGACATAGGGTA | |
| | | CGGTTCGGGGAGTTCGATGCTTTTGATATCTGGGGCCAAGGGACAATGGTC | |
| | | ACCGTCTCTTCA | |
| | VL | CAGGCTGTGCTGACTCAGCCACCCTCAGTGTCTGGGGCCCCCGGAGAGAGG | 80 |
| | (DNA) | GTCACCCTCTCTTGTACAGGGACCAGCTCGAACGTCGGGGCAGATTTTGAT | |
| | | GTACATTGGTACCAGCAGTTTCCAGGAACAGCCCCCAGACTCCTCATCTTT | |
| | | GGTTCCGACAATCGGCCCTCAGGAGTCCCTGACCGATTCTCTGGCTCCAAG | |
| | | TCTGGCACCTCAGCCTCCCTGGCCATCACTGGGCTCCAGGTTGAGGATGAG | |
| | | GCTGATTATTATTGCCAGGCTTATGACGTCAGGCTGAGTGGCTGGGTGTTC | |
| | | GGCGGGGGGACCAAGCTGACCGTCCTA | |
| A-009 | H-CDR1 | SYAIS | 81 |
| | H-CDR2 | GIIPIFGTANYAQKFQG | 82 |
| | H-CDR3 | GRGFGELYFDY | 83 |
| | VH (aa) | QVQLVQSGAEVKKPGSSVKVSCKASGGTFSSYAISWVRQAPGQGLEWMGGI | 84 |
| | | IPIFGTANYAQKFQGRVTITADESTSTAYMELSSLRSEDTAVYYCARGRGF | |
| | | GELYFDYWGQGTLVTVSS | |
| | L-CDR1 | RASQGVRSNIA | 85 |
| | L-CDR2 | DSSTRAT | 86 |
| | L-CDR3 | QQYKNWPRT | 87 |
| | VL (aa) | ETTLTQSPATLSVSPGERATLSCRASQGVRSNIAWYQQKPGQAPRLLIYDS | 88 |
| | | STRATGIPARFSGSGSGTEFTLTISSLQSEDFAVYSCQQYKNWPRTFGQGT | |
| | | KVEIK | |
| | VH | CAGGTTCAGCTGGTGCAGTCTGGGGCTGAGGTGAAGAAGCCTGGGTCCTCG | 89 |
| | (DNA) | GTGAAGGTCTCCTGCAAGGCTTCTGGAGGCACCTTCAGCAGCTATGCTATC | |
| | | AGCTGGGTGCGACAGGCCCCTGGACAAGGGCTTGAGTGGATGGGAGGGATC | |
| | | ATCCCTATCTTTGGTACAGCAAACTACGCACAGAAGTTCCAGGGCAGAGTC | |
| | | ACGATTACCGCGGACGAATCCACGAGCACAGCCTACATGGAGCTGAGCAGC | |
| | | CTGAGATCTGAGGACACGGCCGTGTATTACTGTGCGAGAGGGCGAGGGTTC | |
| | | GGGGAGTTATACTTTGACTACTGGGGCCAGGGAACCCTGGTCACCGTCTCC | |
| | | TCA | |
| | VL | GAAACGACACTCACGCAGTCTCCAGCCACCCTGTCTGTGTCTCCAGGGGAA | 90 |
| | (DNA) | AGAGCCACCCTCTCCTGCAGGGCCAGTCAGGGTGTTAGAAGCAATATAGCC | |
| | | TGGTACCAGCAGAAACCTGGCCAGGCTCCCAGGCTCCTCATCTATGATTCA | |
| | | TCCACCAGGGCCACTGGTATCCCAGCCAGGTTCAGTGGCAGTGGGTCTGGG | |
| | | ACAGAGTTCACTCTCACCATCAGCAGCCTGCAGTCTGAAGATTTTGCAGTT | |
| | | TATTCCTGTCAGCAGTATAAGAACTGGCCTCGGACGTTCGGCCAAGGGACC | |
| | | AAGGTGGAAATCAAA | |
| A-010 | H-CDR1 | SYGIS | 91 |
| | H-CDR2 | WISAYNGNTNYAQKLQG | 92 |
| | H-CDR3 | VPAWSGQFDY | 93 |
| | VH (aa) | QVQLVQSGAEVKKPGASVKVSCKASGYTFTSYGISWVRQAPGQGLEWMGWI | 94 |
| | | SAYNGNTNYAQKLQGRVTMTTDTSTSTAYMELRSLRSDDTAVYYCARVPAW | |
| | | SGQFDYWGQGTLVTVSS | |
| | L-CDR1 | TGSSSDVGGYNYVS | 95 |
| | L-CDR2 | DVRSRPS | 96 |
| | L-CDR3 | TSYTSSNTLVI | 97 |
| | VL (aa) | QSALTQPASVSGSPGQSITISCTGSSSDVGGYNYVSWYQQYPGKAPKLMIY | 98 |
| | | DVRSRPSGVSNRFSGSKSGNTASLTISGLQAEDEADYYCTSYTSSNTLVIF | |
| | | GGGTKVTVL | |
| | VH | CAGGTTCAGCTGGTGCAGTCTGGAGCTGAGGTGAAGAAGCCTGGGGCCTCA | 99 |
| | (DNA) | GTGAAGGTCTCCTGCAAGGCTTCTGGTTACACCTTTACCAGCTATGGTATC | |
| | | AGCTGGGTGCGACAGGCCCCTGGACAAGGGCTTGAGTGGATGGGATGGATC | |
| | | AGCGCCTACAATGGTAACACAAACTATGCACAGAAGCTCCAGGGCAGAGTC | |
| | | ACCATGACCACAGACACATCCACGAGCACAGCCTACATGGAGCTGAGGAGC | |
| | | CTGAGATCTGACGACACGGCCGTGTATTACTGTGCGAGAGTCCCTGCGTGG | |
| | | AGTGGTCAATTTGACTACTGGGGCCAGGGAACCCTGGTCACCGTCTCCTCA | |
| | VL | CAGTCTGCCCTGACTCAGCCTGCCTCCGTGTCTGGGTCTCCTGGACAGTCG | 100 |
| | (DNA) | ATCACCATCTCCTGCACTGGATCCAGCAGTGACGTTGGTGGTTATAACTAT | |
| | | GTCTCCTGGTACCAGCAGTACCCAGGCAAAGCCCCCAAACTCATGATTTAT | |
| | | GATGTCAGAAGTCGGCCCTCAGGGGTTTCTAATCGCTTCTCTGGCTCCAAG | |
| | | TCTGGCAACACGGCCTCCCTGACCATCTCTGGACTCCAGGCTGAGGACGAG | |
| | | GCTGATTATTACTGCACCTCATATACAAGCAGCAACACTCTTGTGATATTC | |
| | | GGCGGAGGGACCAAGGTGACCGTCCTA | |
| A-011 | H-CDR1 | SYAMH | 101 |
| | H-CDR2 | VISYDGSNKYYADSVKG | 102 |
| | H-CDR3 | GGALNYYGMDV | 103 |
| | VH (aa) | QVQLVESGGGVVQPGRSLRLSCAASGFTFSSYAMHWVRQAPGKGLEWVAVI | 104 |
| | | SYDGSNKYYADSVKGRFTISRDNSKNTLYLQMNSLRAEDTAVYYCAEGGAL | |
| | | NYYGMDVWGQGTTVTVSs | |
| | L-CDR1 | QGDSLRHFYAT | 105 |
| | L-CDR2 | GKNNRPS | 106 |
| | L-CDR3 | QSRDPRNNHLI | 107 |
| | VL (aa) | SSELTQDPGVSVALGQTVRITCQGDSLRHFYATWYQQKPGQAPILVIYGKN | 108 |
| | | NRPSGIPDRFSGSSSEDTASLTITGAQAEDEADYYCQSRDPRNNHLIFGGG | |
| | | TKLIVL | |
| | VH | CAGGTGCAGCTGGTGGAGTCTGGGGGAGGCGTGGTCCAGCCTGGGAGGTCC | 109 |
| | (DNA) | CTGAGACTCTCCTGTGCAGCCTCTGGATTCACCTTCAGTAGCTATGCTATG | |
| | | CACTGGGTCCGCCAGGCTCCAGGCAAGGGGCTGGAGTGGGTGGCAGTTATA | |
| | | TCATATGATGGAAGCAATAAATACTACGCAGACTCCGTGAAGGGCCGATTC | |
| | | ACCATCTCCAGAGACAATTCCAAGAACACGCTGTATCTGCAAATGAACAGC | |
| | | CTGAGAGCTGAGGACACGGCTGTGTATTACTGTGCGGAGGGCGGAGCGTTG | |
| | | AACTACTACGGTATGGACGTCTGGGGCCAAGGGACCACGGTCACCGTCTCC | |
| | | TCA | |
| | VL | TCTTCTGAGCTGACTCAGGACCCTGGTGTGTCTGTGGCCTTGGGACAGACA | 110 |
| | (DNA) | GTCAGGATCACATGCCAGGGAGACAGCCTCAGACACTTTTATGCAACCTGG | |
| | | TACCAGCAGAAGCCAGGACAGGCCCCTATACTTGTCATCTATGGTAAAAAC | |
| | | AACCGGCCCTCAGGGATCCCAGACCGATTCTCTGGCTCCAGTTCTGAAGAC | |
| | | ACAGCCTCCTTGACCATCACTGGGGCTCAGGCGGAGGATGAGGCTGACTAC | |
| | | TACTGTCAATCCCGGGACCCCAGGAATAACCATTTGATTTTCGGCGGCGGG | |
| | | ACCAAACTGATCGTCCTC | |
| A-012 | H-CDR1 | SNYMS | 111 |
| | H-CDR2 | VIYSGGSTYYADSVKG | 112 |
| | H-CDR3 | GNPYYYGDLQVNFDY | 113 |
| | VH (aa) | EVQLLESGGGLIQPGGSLRLSCAVSGFTVSSNYMSWVRQAPGKGLEWVSVI | 114 |
| | | YSGGSTYYADSVKGRFTISRDNSKNTLYLQMNSLRAEDTAVYYCARGNPYY | |
| | | YGDLQVNFDYWGQGTLVTVSS | |
| | L-CDR1 | ASSTGAVTSGYYPN | 115 |
| | L-CDR2 | STSNKHS | 116 |
| | L-CDR3 | LLYYGGAWV | 117 |
| | VL (aa) | QAVVTQEPSLTVSPGGTVTLTCASSTGAVTSGYYPNWFQQKPGQAPRALIY | 118 |
| | | STSNKHSWTPARFSGSLLGGKAALTLSGVQPEDEAEYYCLLYYGGAWVFGG | |
| | | GTKLTVL | |
| | VH | GAGGTGCAGCTGTTGGAGTCTGGAGGAGGCTTGATCCAGCCTGGGGGGTCC | 119 |
| | (DNA) | CTGAGACTCTCCTGTGCAGTCTCTGGGTTCACCGTCAGTAGCAACTACATG | |
| | | AGCTGGGTCCGCCAGGCTCCAGGGAAGGGGCTGGAGTGGGTCTCAGTTATT | |
| | | TATAGCGGTGGTAGCACATACTACGCAGACTCCGTGAAGGGCCGATTCACC | |
| | | ATCTCCAGAGACAATTCCAAGAACACGCTGTATCTTCAAATGAACAGCCTG | |
| | | AGAGCCGAGGACACGGCCGTGTATTACTGTGCGAGAGGTAACCCATATTAC | |
| | | TACGGTGACCTCCAGGTGAACTTTGACTACTGGGGCCAGGGAACCCTGGTC | |
| | | ACCGTCTCCTCA | |
| | VL | CAGGCTGTGGTGACTCAGGAGCCCTCACTGACTGTGTCCCCAGGAGGGACA | 120 |
| | (DNA) | GTCACTCTCACCTGTGCTTCCAGCACTGGAGCAGTCACCAGTGGTTACTAT | |
| | | CCAAACTGGTTCCAGCAGAAACCTGGACAAGCACCCAGGGCACTGATTTAT | |
| | | AGTACAAGCAACAAACACTCCTGGACCCCTGCCCGGTTCTCAGGCTCCCTC | |
| | | CTTGGGGGCAAAGCTGCCCTGACACTGTCAGGTGTGCAGCCTGAGGACGAG | |
| | | GCTGAGTATTACTGCCTGCTCTACTATGGTGGTGCTTGGGTGTTCGGCGGA | |
| | | GGGACCAAGCTGACCGTCCTA | |
| A-013 | H-CDR1 | SNYMS | 121 |
| | H-CDR2 | VIYSGGSTYYADSVKG | 122 |
| | H-CDR3 | GNPYYYGDLQVNFDY | 123 |
| | VH (aa) | EVQLVESGGGLIQPGGSLRLSCAASGFTVSSNYMSWVRQAPGKGLEWVSVI | 124 |
| | | YSGGSTYYADSVKGRFTISRDNSKNTLYLQMNSLRAEDTAVYYCARGNPYY | |
| | | YGDLQVNFDYWGQGTLVTVSS | |
| | L-CDR1 | ASSTGAVTSAYYPN | 125 |
| | L-CDR2 | STSNKHS | 126 |
| | L-CDR3 | LLYYGGAWV | 127 |
| | VL (aa) | QAVVTQEPSLTVSPGGTVTLTCASSTGAVTSAYYPNWFQQKPGQAPRALIY | 128 |
| | | STSNKHSWTPARFSGSLLGGKAALTLSGVQPEDEADYYCLLYYGGAWVFGG | |
| | | GTKLTVL | |
| | VH | GAAGTGCAGCTGGTGGAGTCTGGAGGAGGCTTGATCCAGCCTGGGGGGTCC | 129 |
| | (DNA) | CTGAGACTCTCCTGTGCAGCCTCTGGGTTCACCGTCAGTAGCAACTACATG | |
| | | AGCTGGGTCCGCCAGGCTCCAGGGAAGGGGCTGGAGTGGGTCTCAGTTATT | |
| | | TATAGCGGTGGTAGCACATACTACGCAGACTCCGTGAAGGGCCGATTCACC | |
| | | ATCTCCAGAGACAATTCCAAGAACACGCTGTATCTTCAAATGAACAGCCTG | |
| | | AGAGCCGAGGACACGGCCGTGTATTACTGTGCGAGAGGTAACCCATATTAC | |
| | | TACGGTGACCTCCAGGTGAACTTTGACTACTGGGGCCAGGGAACCCTGGTC | |
| | | ACCGTCTCCTCA | |
| | VL | CAGGCTGTGGTGACTCAGGAGCCCTCACTGACTGTGTCCCCAGGAGGGACA | 130 |
| | (DNA) | GTCACTCTCACCTGTGCTTCCAGCACTGGAGCAGTCACCAGTGCTTACTAT | |
| | | CCAAACTGGTTCCAGCAGAAACCTGGACAAGCACCCAGGGCACTGATTTAT | |
| | | AGTACAAGCAACAAACACTCCTGGACCCCTGCCCGGTTCTCAGGCTCCCTC | |
| | | CTTGGGGGCAAAGCTGCCCTGACACTGTCAGGTGTGCAGCCTGAGGACGAG | |
| | | GCTGACTATTACTGCCTGCTCTACTATGGTGGTGCTTGGGTGTTCGGCGGA | |
| | | GGGACCAAGCTGACCGTCCTA | |
| A-014 | H-CDR1 | DYYIH | 131 |
| | H-CDR2 | WFNPSTGGANYAQKFQG | 132 |
| | H-CDR3 | GNSPDLDY | 133 |
| | VH (aa) | QVQLVQSGAEVKKPGASVKVSCKASGYTFNDYYIHWVRQAPGQGLEWMGWF | 134 |
| | | NPSTGGANYAQKFQGRVTMTRDTSISTAYLEVSSLRSDDTAVYYCARGNSP | |
| | | DLDYWGQGTLVTVSS | |
| | L-CDR1 | KSSQSLLHSSNNKNYLA | 135 |
| | L-CDR2 | WASTRQS | 136 |
| | L-CDR3 | QQYYTTTPNT | 137 |
| | VL (aa) | DIQMTQSPDSLTVSLGERATINCKSSQSLLHSSNNKNYLAWYQQRPGQPPK | 138 |
| | | LLIYWASTRQSGVPDRFSGSGSGTDFTLTINSLQADDMAVYYCQQYYTTTP | |
| | | NTFGQGTKLEIK | |
| | VH | CAAGTCCAGCTGGTGCAGTCTGGGGCTGAGGTGAAGAAGCCTGGGGCCTCA | 139 |
| | (DNA) | GTGAAGGTCTCCTGCAAGGCTTCTGGATACACCTTCAACGACTACTATATA | |
| | | CACTGGGTGCGGCAGGCCCCTGGACAAGGGCTTGAGTGGATGGGGTGGTTC | |
| | | AACCCTAGCACTGGTGGCGCAAATTATGCACAGAAGTTTCAGGGCAGGGTC | |
| | | ACCATGACCAGGGACACGTCCATCAGCACAGCCTACTTGGAAGTGAGCAGC | |
| | | CTGAGATCTGACGACACGGCCGTGTATTACTGTGCGAGAGGTAATAGCCCG | |
| | | GACCTTGACTACTGGGGCCAGGGAACCCTGGTCACCGTCTCCTCA | |
| | VL | GACATCCAGATGACCCAGTCTCCAGACTCCCTGACTGTGTCTCTGGGCGAG | 140 |
| | (DNA) | AGGGCCACCATCAACTGCAAGTCGAGCCAGAGTCTTTTACACAGCTCCAAC | |
| | | AATAAGAATTACTTGGCTTGGTACCAGCAGAGACCAGGACAGCCTCCTAAA | |
| | | CTGCTCATTTACTGGGCATCCACCCGGCAATCCGGGGTCCCGGACCGCTTC | |
| | | AGTGGCAGCGGGTCTGGGACAGATTTCACTCTCACCATCAACAGCCTGCAG | |
| | | GCTGACGACATGGCAGTTTATTACTGCCAGCAGTATTATACTACTACTCCG | |
| | | AACACTTTTGGCCAGGGGACCAAGCTGGAGATCAAA | |
| A-015 | H-CDR1 | GYYMH | 141 |
| | H-CDR2 | WINPNSGGTNYAQKFQG | 142 |
| | H-CDR3 | DPDGSGGSSRWFDP | 143 |
| | VH (aa) | QVQLVQSGAEVKKPGASVKVSCKASGYTFTGYYMHWVRQAPGQGLEWMGW1 | 144 |
| | | NPNSGGTNYAQKFQGRVTMTRDTSISTAYMELSRLRSDDTAVYYCARDPDG | |
| | | SGGSSRWFDPWGQGTLVTVSS | |
| | L-CDR1 | TLSSGHTNYAIA | 145 |
| | L-CDR2 | LNSDGSHTRGG | 146 |
| | L-CDR3 | MIWHNNAWV | 147 |
| | VL (aa) | QPVLTQSPSASASLGASVKLTCTLSSGHTNYAIAWRQQQPGKAPRYLMLLN | 148 |
| | | SDGSHTRGGGIPDRFSGSSSGAERYLTISSLQSEDEADYYCMIWHNNAWVF | |
| | | GGGTKLAVL | |
| | VH | CAAGTGCAGCTGGTGCAGTCTGGGGCTGAGGTGAAGAAGCCTGGGGCCTCA | 149 |
| | (DNA) | GTGAAGGTCTCCTGCAAGGCTTCTGGATACACCTTCACCGGCTACTATATG | |
| | | CACTGGGTGCGACAGGCCCCTGGACAAGGGCTTGAGTGGATGGGATGGATC | |
| | | AACCCTAACAGTGGTGGCACAAACTATGCACAGAAGTTTCAGGGCAGGGTC | |
| | | ACCATGACCAGGGACACGTCCATCAGCACAGCCTACATGGAGCTGAGCAGG | |
| | | CTGAGATCTGACGACACGGCCGTGTATTACTGTGCGAGAGATCCTGATGGG | |
| | | AGTGGTGGTAGTTCCCGGTGGTTCGACCCCTGGGGCCAGGGAACCCTGGTC | |
| | | ACCGTCTCCTCA | |
| | VL | CAGCCTGTGCTGACTCAATCGCCCTCTGCCTCTGCCTCCCTGGGAGCCTCG | 150 |
| | (DNA) | GTCAAGCTCACCTGCACTCTGAGCAGTGGGCACACCAACTACGCCATCGCT | |
| | | TGGCGTCAGCAACAGCCTGGGAAGGCCCCTCGATATTTGATGCTGCTTAAC | |
| | | AGTGATGGCAGCCACACGAGGGGGGGCGGGATCCCTGATCGCTTCTCAGGC | |
| | | TCCAGTTCTGGGGCTGAGCGCTACCTCACCATCTCCAGCCTCCAGTCTGAG | |
| | | GATGAGGCTGACTATTACTGTATGATTTGGCACAACAACGCTTGGGTGTTC | |
| | | GGCGGAGGGACCAAGCTGGCCGTCCTT | |
| A-016 | H-CDR1 | GYYMH | 151 |
| | H-CDR2 | GIIPIFGTANYAQKFQG | 152 |
| | H-CDR3 | AGMELTRSGAYYYYGMDV | 153 |
| | VH (aa) | QVQLQESGAEVKKPGASVKVSCKASGYTFTGYYMHWVRQAPGQGLEWMGGI | 154 |
| | | IPIFGTANYAQKFQGRVTITADESTSTAYMELSSLRSEDTAVYYCAGAGME | |
| | | LTRSGAYYYYGMDVWGQGTTVTVSS | |
| | L-CDR1 | TGTSSDIGGYTFVS | 155 |
| | L-CDR2 | DVNNRPS | 156 |
| | L-CDR3 | SSVTSTNTYV | 157 |
| | VL (aa) | QSALTQPASVSGSPGQSITISCTGTSSDIGGYTFVSWYQQHPGKAPKVMIH | 158 |
| | | DVNNRPSGISNRFSGSKSGNTASLTISGLQAEDEADYYCSSVTSTNTYVFG | |
| | | TGTKVTVL | |
| | VH | CAGGTGCAGCTGCAGGAGTCGGGGGCTGAGGTGAAGAAGCCTGGGGCCTCA | 159 |
| | (DNA) | GTGAAGGTCTCCTGCAAGGCTTCTGGATACACCTTCACCGGCTACTATATG | |
| | | CACTGGGTGCGACAGGCCCCTGGACAAGGGCTTGAGTGGATGGGAGGGATC | |
| | | ATCCCTATCTTTGGTACAGCAAACTACGCACAGAAGTTCCAGGGCAGAGTC | |
| | | ACGATTACCGCGGACGAATCCACGAGCACAGCCTACATGGAGCTGAGCAGC | |
| | | CTGAGATCTGAGGACACGGCCGTGTATTACTGTGCCGGGGCGGGGATGGAG | |
| | | CTTACTCGCTCGGGTGCTTACTACTACTACGGTATGGACGTCTGGGGCCAA | |
| | | GGGACCACGGTCACCGTCTCCTCA | |
| | VL | CAGTCTGCCCTGACTCAGCCTGCCTCCGTGTCTGGGTCTCCTGGACAGTCG | 160 |
| | (DNA) | ATCACCATCTCCTGCACTGGAACCAGCAGTGACATTGGTGGTTATACCTTT | |
| | | GTCTCCTGGTACCAACAACACCCAGGCAAAGCCCCCAAAGTCATGATTCAT | |
| | | GATGTCAATAATCGGCCCTCAGGGATTTCTAATCGCTTCTCTGGCTCCAAG | |
| | | TCTGGCAACACGGCCTCCCTGACCATCTCTGGGCTCCAGGCTGAGGACGAG | |
| | | GCTGATTATTACTGCAGCTCAGTTACAAGCACCAACACTTATGTCTTCGGA | |
| | | ACTGGGACAAAGGTCACCGTCCTA | |
| A-017 | H-CDR1 | GYYMH | 161 |
| | H-CDR2 | WINPNSGNTGYAQKFQG | 162 |
| | H-CDR3 | GGQQQLVLDDY | 163 |
| | VH (aa) | QVQLVQSGAEVKKPGASVKVSCKASGYTFTGYYMHWVRQAPGQGLEWMGW1 | 164 |
| | | NPNSGNTGYAQKFQGRVTMTRNTSISTAYMELSSLRSEDTAVYYCARGGQQ | |
| | | QLVLDDYWGQGTLVTVSS | |
| | L-CDR1 | QASQDIRHHLN | 165 |
| | L-CDR2 | DSSNLET | 166 |
| | L-CDR3 | QQYDSLPRT | 167 |
| | VL (aa) | DIVLTQSPASLSASVGDRITITCQASQDIRHHLNWFQHKPGKAPKLLISDS | 168 |
| | | SNLETGVPSRFSGSGSGTDFSFTISRLQPEDIATYYCQQYDSLPRTFGQGT | |
| | | KLEIK | |
| | VH | CAGGTGCAGCTGGTGCAATCTGGGGCTGAGGTGAAGAAGCCTGGGGCCTCA | 169 |
| | (DNA) | GTGAAGGTCTCCTGCAAGGCTTCTGGATACACCTTCACCGGCTACTATATG | |
| | | CACTGGGTGCGACAGGCCCCTGGACAAGGGCTTGAGTGGATGGGATGGATC | |
| | | AACCCTAACAGTGGTAACACAGGCTATGCACAGAAGTTCCAGGGCAGAGTC | |
| | | ACCATGACCAGGAACACCTCCATAAGCACAGCCTACATGGAGCTGAGCAGC | |
| | | CTGAGATCTGAGGACACGGCCGTGTATTACTGTGCGAGAGGCGGACAGCAG | |
| | | CAGCTGGTACTGGACGACTACTGGGGCCAGGGAACCCTGGTCACCGTCTCC | |
| | | TCA | |
| | VL | GATATTGTGCTGACACAGTCTCCAGCCTCCCTGTCTGCATCTGTCGGCGAC | 170 |
| | (DNA) | AGGATCACCATCACCTGCCAGGCGAGTCAGGACATTAGGCATCATTTAAAT | |
| | | TGGTTTCAGCACAAACCAGGGAAAGCCCCCAAGCTCCTGATCTCCGATTCA | |
| | | TCCAACCTGGAAACAGGAGTCCCGTCAAGGTTCAGTGGAAGTGGGTCTGGG | |
| | | ACAGATTTTTCTTTCACCATCAGCCGCCTGCAGCCTGAAGATATTGCGACT | |
| | | TATTACTGTCAACAATATGATAGTCTGCCTCGAACCTTTGGCCAGGGGACC | |
| | | AAACTGGAGATCAAA | |
| A-018 | H-CDR1 | SYAMH | 171 |
| | H-CDR2 | VISYDGSNKYYADSVKG | 172 |
| | H-CDR3 | ASPSQWLVLGHY | 173 |
| | VH (aa) | QVQLVESGGGVVQPGRSLRLSCAASGFTFSSYAMHWVRQAPGKGLEWVAVI | 174 |
| | | SYDGSNKYYADSVKGRFTISRDNSKNTLYLQMNSLRAEDTAVYYCARASPS | |
| | | QWLVLGHYWGQGTLVTVSS | |
| | L-CDR1 | RASQSVSSSYLA | 175 |
| | L-CDR2 | GASSRAT | 176 |
| | L-CDR3 | QQYGSSPWT | 177 |
| | VL (aa) | ETTLTQSPGTLSLSPGERATLSCRASQSVSSSYLAWYQQKPGQAPRLLIYG | 178 |
| | | ASSRATGIPDRFSGSGSGTDFTLTISRLEPEDFAVYYCQQYGSSPWTFGQG | |
| | | TKVEIK | |
| | VH | CAGGTGCAGCTGGTGGAGTCTGGGGGAGGCGTGGTCCAGCCTGGGAGGTCC | 179 |
| | (DNA) | CTGAGACTCTCCTGTGCAGCCTCTGGATTCACCTTCAGTAGCTATGCTATG | |
| | | CACTGGGTCCGCCAGGCTCCAGGCAAGGGGCTGGAGTGGGTGGCAGTTATA | |
| | | TCATATGATGGAAGCAATAAATACTACGCAGACTCCGTGAAGGGCCGATTC | |
| | | ACCATCTCCAGAGACAATTCCAAGAACACGCTGTATCTGCAAATGAACAGC | |
| | | CTGAGAGCTGAGGACACGGCTGTGTATTACTGTGCGAGAGCCTCTCCATCA | |
| | | CAGTGGCTGGTACTCGGGCACTACTGGGGCCAGGGAACCCTGGTCACCGTC | |
| | | TCCTCA | |
| | VL | GAAACGACACTCACGCAGTCTCCAGGCACCCTGTCTTTGTCTCCAGGGGAA | 180 |
| | (DNA) | AGAGCCACCCTCTCCTGCAGGGCCAGTCAGAGTGTTAGCAGCAGCTACTTA | |
| | | GCCTGGTACCAGCAGAAACCTGGCCAGGCTCCCAGGCTCCTCATCTATGGT | |
| | | GCATCCAGCAGGGCCACTGGCATCCCAGACAGGTTCAGTGGCAGTGGGTCT | |
| | | GGGACAGACTTCACTCTCACCATCAGCAGACTGGAGCCTGAAGATTTTGCA | |
| | | GTGTATTACTGTCAGCAGTATGGTAGCTCACCGTGGACGTTCGGCCAAGGG | |
| | | ACCAAGGTGGAAATCAAA | |
| A-019 | H-CDR1 | GYYMH | 181 |
| | H-CDR2 | WINPNSGNTGYAQKFQG | 182 |
| | H-CDR3 | GGQQQLVLDDY | 183 |
| | VH (aa) | QVQLVQSGAEVKKPGASVKVSCKASGYTFTGYYMHWVRQAPGQGLEWMGW1 | 184 |
| | | NPNSGNTGYAQKFQGRVTMTRNTSISTAYMELSSLRSEDTAVYYCARGGQQ | |
| | | QLVLDDYWGQGTLVTVSS | |
| | L-CDR1 | RASQSVSSNLA | 185 |
| | L-CDR2 | GASTRAT | 186 |
| | L-CDR3 | QQYNNWPWT | 187 |
| | VL (aa) | ETTLTQSPATLSVSPGERATLSCRASQSVSSNLAWYQQKPGQAPRLLIYGA | 188 |
| | | STRATGIPARFSGSGSGTEFTLTISSLQSEDFAVYYCQQYNNWPWTFGQGT | |
| | | KVETK | |
| | VH | CAGGTGCAGCTGGTGCAGTCTGGGGCTGAGGTGAAGAAGCCTGGGGCCTCA | 189 |
| | (DNA) | GTGAAGGTCTCCTGCAAGGCTTCTGGATACACCTTCACCGGCTACTATATG | |
| | | CACTGGGTGCGACAGGCCCCTGGACAAGGGCTTGAGTGGATGGGATGGATC | |
| | | AACCCTAACAGTGGTAACACAGGCTATGCACAGAAGTTCCAGGGCAGAGTC | |
| | | ACCATGACCAGGAACACCTCCATAAGCACAGCCTACATGGAGCTGAGCAGC | |
| | | CTGAGATCTGAGGACACGGCCGTGTATTACTGTGCGAGAGGCGGACAGCAG | |
| | | CAGCTGGTACTGGACGACTACTGGGGCCAGGGAACCCTGGTCACCGTCTCC | |
| | | TCA | |
| | VL | GAAACGACACTCACGCAGTCTCCAGCCACCCTGTCTGTGTCTCCAGGGGAA | 190 |
| | (DNA) | AGAGCCACCCTCTCCTGCAGGGCCAGTCAGAGTGTTAGCAGCAACTTAGCC | |
| | | TGGTACCAGCAGAAACCTGGCCAGGCTCCCAGGCTCCTCATCTATGGTGCA | |
| | | TCCACCAGGGCCACTGGTATCCCAGCCAGGTTCAGTGGCAGTGGGTCTGGG | |
| | | ACAGAGTTCACTCTCACCATCAGCAGCCTGCAGTCTGAAGATTTTGCAGTT | |
| | | TATTACTGTCAGCAGTATAATAACTGGCCTTGGACGTTCGGCCAAGGGACC | |
| | | AAGGTGGAAACCAAA | |
| A-020 | H-CDR1 | SGGYYWS | 191 |
| | H-CDR2 | YIYYSGSTYYNPSLKS | 192 |
| | H-CDR3 | GGISPSGSSIYYYYGMDV | 193 |
| | VH (aa) | QLQLQESGPGLVKPSQTLSLTCTVSGGSISSGGYYWSWIRQHPGKGLEWIG | 194 |
| | | YIYYSGSTYYNPSLKSRVTISVDTSKNQFSLKLSSVTAADTAVYYCARGGI | |
| | | SPSGSSIYYYYGMDVWGQGTTVTVSS | |
| | L-CDR1 | TGTSNDVGGYNYVS | 195 |
| | L-CDR2 | EVNKRPS | 196 |
| | L-CDR3 | SSYAGTKEV | 197 |
| | VL (aa) | QPVLTQPPSASGSPGQSVTISCTGTSNDVGGYNYVSWYQQHPGKAPKLIIY | 198 |
| | | EVNKRPSGVPDRFSGSKSGNTASLTVSGLQAVDESDYYCSSYAGTKEVFGG | |
| | | GTKLTVL | |
| | VH | CAGCTGCAGCTGCAGGAGTCCGGCCCAGGACTGGTGAAGCCTTCACAGACC | 199 |
| | (DNA) | CTGTCCCTCACCTGCACTGTCTCTGGTGGCTCCATCAGCAGTGGTGGTTAC | |
| | | TACTGGAGCTGGATCCGCCAGCACCCAGGGAAGGGCCTGGAGTGGATTGGG | |
| | | TACATCTATTACAGTGGGAGCACCTACTACAACCCGTCCCTCAAGAGTCGA | |
| | | GTTACCATATCAGTAGACACGTCTAAGAACCAGTTCTCCCTGAAGCTGAGC | |
| | | TCTGTGACTGCCGCGGACACGGCCGTGTATTACTGTGCGAGAGGTGGGATA | |
| | | TCACCCTCCGGGTCCTCGATCTACTACTACTACGGTATGGACGTCTGGGGC | |
| | | CAAGGGACCACGGTCACCGTCTCCTCA | |
| | VL | CAGCCTGTGCTGACTCAGCCACCCTCCGCGTCCGGGTCTCCTGGACAGTCA | 200 |
| | (DNA) | GTCACCATCTCCTGCACTGGAACCAGCAATGACGTTGGTGGTTATAACTAT | |
| | | GTCTCCTGGTACCAACAACACCCAGGCAAAGCCCCCAAACTCATAATTTAT | |
| | | GAGGTCAATAAGCGGCCCTCAGGGGTCCCTGATCGCTTCTCTGGCTCCAAG | |
| | | TCTGGCAACACGGCCTCCCTGACCGTCTCTGGGCTCCAGGCTGTGGATGAG | |
| | | TCTGATTATTACTGCAGCTCATATGCAGGCACCAAGGAGGTCTTCGGCGGA | |
| | | GGGACCAAGCTGACCGTCCTA | |
| A-021 | H-CDR1 | SYGIS | 201 |
| | H-CDR2 | WISAYNGNTNYAQKLQG | 202 |
| | H-CDR3 | VPAWSGQFDY | 203 |
| | VH (aa) | QVQLVQSGAEVKKPGASVKVSCKASGYTFTSYGISWVRQAPGQGLEWMGWI | 204 |
| | | SAYNGNTNYAQKLQGRVTMTTDTSTSTAYMELRSLRSDDTAVYYCARVPAW | |
| | | SGQFDYWGQGTLVTVSS | |
| | L-CDR1 | TGTSSDVGGYNYVS | 205 |
| | L-CDR2 | EVTNRPS | 206 |
| | L-CDR3 | NSYTSGPTYVL | 207 |
| | VL (aa) | QSALTQPASVSGSPGQSITISCTGTSSDVGGYNYVSWYQQHPGKAPKLMIY | 208 |
| | | EVTNRPSGVSDRFSGSKSANTASLTISELQAEDEAVYYCNSYTSGPTYVLF | |
| | | GGGTQLTVL | |
| | VH | CAGGTGCAGCTGGTGCAGTCTGGAGCTGAGGTGAAGAAGCCTGGGGCCTCA | 209 |
| | (DNA) | GTGAAGGTCTCCTGCAAGGCTTCTGGTTACACCTTTACCAGCTATGGTATC | |
| | | AGCTGGGTGCGACAGGCCCCTGGACAAGGGCTTGAGTGGATGGGATGGATC | |
| | | AGCGCTTACAATGGTAACACAAACTATGCACAGAAGCTCCAGGGCAGAGTC | |
| | | ACCATGACCACAGACACATCCACGAGCACAGCCTACATGGAGCTGAGGAGC | |
| | | CTGAGATCTGACGACACGGCCGTGTATTACTGTGCGAGAGTCCCTGCGTGG | |
| | | AGTGGTCAATTTGACTACTGGGGCCAGGGAACCCTGGTCACCGTCTCCTCA | |
| | VL | CAGTCTGCCCTGACTCAGCCTGCCTCCGTGTCTGGGTCTCCTGGACAGTCG | 210 |
| | (DNA) | ATCACCATCTCCTGCACTGGAACCAGCAGTGACGTTGGTGGTTATAACTAT | |
| | | GTCTCCTGGTACCAACAGCACCCAGGCAAAGCCCCCAAACTCATGATTTAT | |
| | | GAGGTCACTAATCGGCCCTCAGGGGTTTCCGATCGCTTCTCTGGCTCCAAG | |
| | | TCTGCCAACACGGCCTCCCTGACCATCTCTGAGCTCCAGGCTGAAGACGAG | |
| | | GCTGTTTATTACTGCAACTCATACACAAGCGGCCCCACTTATGTGCTGTTC | |
| | | GGCGGAGGGACCCAGCTGACCGTCCTA | |
| A-022 | H-CDR1 | SGGYYWS | 211 |
| | H-CDR2 | YIYYSGSTYYNPSLKS | 212 |
| | H-CDR3 | ASRSTDYYFDY | 213 |
| | VH (aa) | EVQLLESGPGLVKPSQTLSLTCTVSGGSISSGGYYWSWIRQHPGKGLEWIG | 214 |
| | | YIYYSGSTYYNPSLKSRVTISVDTSKNQFSLKLSSVTAADTAVYYCARASR | |
| | | STDYYFDYWGQGTLVTVSS | |
| | L-CDR1 | TGNRNNIGDQGAA | 215 |
| | L-CDR2 | RNNNGPS | 216 |
| | L-CDR3 | SAWDSSLRAWV | 217 |
| | VL (aa) | QPGLTQPPSMSYGLGQTATLTCTGNRNNIGDQGAAWLQQHQGHPPKLLSYR | 218 |
| | | NNNGPSGISERLSASRSGNTASLTISGLQPEDEADYYCSAWDSSLRAWVFG | |
| | | GGTKLTVL | |
| | VH | GAAGTGCAGCTGTTGGAGTCTGGCCCAGGACTGGTGAAGCCTTCACAGACC | 219 |
| | (DNA) | CTGTCCCTCACCTGCACTGTCTCTGGTGGCTCCATCAGCAGTGGTGGTTAC | |
| | | TACTGGAGCTGGATCCGCCAGCACCCAGGGAAGGGCCTGGAGTGGATTGGG | |
| | | TACATCTATTACAGTGGGAGCACCTACTACAACCCGTCCCTCAAGAGTCGA | |
| | | GTTACCATATCAGTAGACACGTCTAAGAACCAGTTCTCCCTGAAGCTGAGC | |
| | | TCTGTGACTGCCGCGGACACGGCCGTGTATTACTGTGCGAGGGCTTCACGA | |
| | | TCGACCGATTACTACTTTGACTACTGGGGCCAGGGAACCCTGGTCACCGTC | |
| | | TCCTCA | |
| | VL | CAGCCAGGGCTGACTCAGCCACCCTCGATGTCCTACGGCTTGGGACAGACC | 220 |
| | (DNA) | GCCACACTCACCTGCACTGGGAACAGAAACAATATTGGCGACCAAGGAGCA | |
| | | GCTTGGCTGCAGCAGCACCAGGGCCACCCTCCCAAACTCCTATCCTACAGG | |
| | | AATAACAACGGGCCCTCAGGGATCTCAGAGAGATTATCTGCATCCAGGTCA | |
| | | GGAAACACAGCCTCCCTGACCATTAGTGGACTCCAGCCTGAGGACGAGGCT | |
| | | GACTATTACTGCTCAGCATGGGACAGCAGCCTCAGGGCTTGGGTGTTCGGC | |
| | | GGAGGGACCAAGCTGACCGTCCTC | |
| A-023 | H-CDR1 | SYAIS | 221 |
| | H-CDR2 | GIIPIFGTANYAQKFQG | 222 |
| | H-CDR3 | PKYSSGWFYYYGMDV | 223 |
| | VH (aa) | QVQLVQSGAEVKKPGSSVKVSCKASGGTFSSYAISWVRQAPGQGLEWMGGI | 224 |
| | | IPIFGTANYAQKFQGRVTITADESTSTAYMELSSLRSEDTAVYYCARPKYS | |
| | | SGWFYYYGMDVWGQGTTVTVSS | |
| | L-CDR1 | SGSSSNIGNNYVS | 225 |
| | L-CDR2 | DNNKRPS | 226 |
| | L-CDR3 | GTWDSSLSW | 227 |
| | VL (aa) | QSVLTQPPSVSAAPGQKVTISCSGSSSNIGNNYVSWYQQLPGTAPKLLIYD | 228 |
| | | NNKRPSGIPDRFSGSKSGTSATLGITGLQTGDEADYYCGTWDSSLSVVFGG | |
| | | GTKLTVL | |
| | VH | CAGGTCCAGCTGGTGCAGTCTGGGGCTGAGGTGAAGAAGCCTGGGTCCTCG | 229 |
| | (DNA) | GTGAAGGTCTCCTGCAAGGCTTCTGGAGGCACCTTCAGCAGCTATGCTATC | |
| | | AGCTGGGTGCGACAGGCCCCTGGACAAGGGCTTGAGTGGATGGGAGGGATC | |
| | | ATCCCTATCTTTGGTACAGCAAACTACGCACAGAAGTTCCAGGGCAGAGTC | |
| | | ACGATTACCGCGGACGAATCCACGAGCACAGCCTACATGGAGCTGAGCAGC | |
| | | CTGAGATCTGAGGACACGGCCGTGTATTACTGTGCGAGACCAAAGTATAGC | |
| | | AGTGGCTGGTTCTACTACTACGGTATGGACGTCTGGGGCCAAGGGACCACG | |
| | | GTCACCGTCTCCTCA | |
| | VL | CAGTCTGTGCTGACTCAGCCACCCTCAGTGTCTGCGGCCCCAGGACAGAAG | 230 |
| | (DNA) | GTCACCATCTCCTGCTCTGGAAGCAGCTCCAACATTGGGAATAATTATGTA | |
| | | TCCTGGTACCAGCAGCTCCCAGGAACAGCCCCCAAACTCCTCATTTATGAC | |
| | | AATAATAAGCGACCCTCAGGGATTCCTGACCGATTCTCTGGCTCCAAGTCT | |
| | | GGCACGTCAGCCACCCTGGGCATCACCGGACTCCAGACTGGGGACGAGGCC | |
| | | GATTATTACTGCGGAACATGGGATAGCAGCCTGAGTGTGGTATTCGGCGGA | |
| | | GGGACCAAGCTGACCGTCCTA | |
| A-024 | H-CDR1 | SYAIS | 231 |
| | H-CDR2 | WISAYNGNTNYAQKLQG | 232 |
| | H-CDR3 | LGSYGYTGAFDI | 233 |
| | VH (aa) | QMQLVQSGAEVKKPGSSVKVSCKASGGTFSSYAISWVRQAPGQGLEWMGWI | 234 |
| | | SAYNGNTNYAQKLQGRVTMTTDTSTSTAYMELRSLRSDDTAVYYCASLGSY | |
| | | GYTGAFDIWGQGTMATVSS | |
| | L-CDR1 | TGTSSDVGGYNYVS | 235 |
| | L-CDR2 | EVSNRPS | 236 |
| | L-CDR3 | SSYTSSSTLW | 237 |
| | VL (aa) | QSALTQPASVSGSPGQSITISCTGTSSDVGGYNYVSWYQQHPGKAPKLMIY | 238 |
| | | EVSNRPSGVSNRFSGSKSGNTASLTISGLQAEDEADYYCSSYTSSSTLVVF | |
| | | GGGTKLTVL | |
| | VH | CAAATGCAGCTGGTACAATCTGGGGCTGAGGTGAAGAAGCCTGGGTCCTCG | 239 |
| | (DNA) | GTGAAGGTCTCCTGCAAGGCTTCTGGAGGCACCTTCAGCAGCTATGCTATC | |
| | | AGCTGGGTGCGACAGGCCCCTGGACAAGGGCTTGAGTGGATGGGATGGATC | |
| | | AGCGCTTACAATGGTAACACAAACTATGCACAGAAGCTCCAGGGCAGAGTC | |
| | | ACCATGACCACAGACACATCCACGAGCACAGCCTACATGGAGCTGAGGAGC | |
| | | CTGAGATCTGACGACACGGCCGTGTATTACTGTGCGAGCCTGGGGAGCTAT | |
| | | GGTTATACAGGGGCTTTTGATATCTGGGGCCAAGGGACAATGGCCACCGTC | |
| | | TCTTCA | |
| | VL | CAGTCTGCCCTGACTCAGCCTGCCTCCGTGTCTGGGTCTCCTGGACAGTCG | 240 |
| | (DNA) | ATCACCATCTCCTGCACTGGAACCAGCAGTGACGTTGGTGGTTATAACTAT | |
| | | GTCTCCTGGTACCAACAGCACCCAGGCAAAGCCCCCAAACTCATGATTTAT | |
| | | GAGGTCAGTAATCGGCCCTCAGGGGTTTCTAATCGCTTCTCTGGCTCCAAG | |
| | | TCTGGCAACACGGCCTCCCTGACCATCTCTGGGCTCCAGGCTGAGGACGAG | |
| | | GCTGATTATTACTGCAGCTCATATACAAGCAGCAGCACTCTCGTGGTATTC | |
| | | GGCGGAGGGACCAAGCTGACCGTCCTA | |
| A-025 | H-CDR1 | SYAIS | 241 |
| | H-CDR2 | GIIPIFGTANYAQKFQG | 242 |
| | H-CDR3 | GGWLRQNWFDP | 243 |
| | VH (aa) | QVQLVQSGAEVKKPGSSVKVSCKASGGTFSSYAISWVRQAPGQGLEWMGGI | 244 |
| | | IPIFGTANYAQKFQGRVTITADESTSTAYMELTSLRSEDTAVYYCARGGWL | |
| | | RQNWFDPWGQGTLVTVSS | |
| | L-CDR1 | QGDSLRSYYAS | 245 |
| | L-CDR2 | GKNNRPS | 246 |
| | L-CDR3 | NSRDSSGNHPRV | 247 |
| | VL (aa) | SSELTQDPAVSVALGQTVRITCQGDSLRSYYASWYQQKPGQAPVLVIYGKN | 248 |
| | | NRPSGIPDRFSGSSSGNTASLTITGAQAEDEADYYCNSRDSSGNHPRVFGG | |
| | | GTKLTVL | |
| | VH | CAGGTGCAGCTGGTGCAGTCTGGGGCTGAGGTGAAGAAGCCTGGGTCCTCG | 249 |
| | (DNA) | GTGAAGGTCTCCTGCAAGGCTTCTGGAGGCACCTTCAGCAGCTATGCTATC | |
| | | AGCTGGGTGCGACAGGCCCCTGGACAAGGGCTTGAGTGGATGGGAGGGATC | |
| | | ATCCCTATCTTTGGTACAGCAAACTACGCACAGAAGTTCCAGGGCAGAGTC | |
| | | ACGATAACCGCGGACGAATCCACGAGCACAGCCTACATGGAGCTGACCAGC | |
| | | CTGAGATCTGAGGACACGGCCGTGTATTACTGTGCGAGAGGGGGGTGGCTA | |
| | | CGACAGAACTGGTTCGACCCCTGGGGCCAGGGAACCCTGGTCACCGTCTCC | |
| | | TCA | |
| | VL | TCTTCTGAGCTGACTCAGGACCCTGCTGTGTCTGTGGCCTTGGGACAAACA | 250 |
| | (DNA) | GTCAGGATCACATGCCAAGGAGACAGCCTCAGAAGGTATTATGCAAGCTGG | |
| | | TACCAGCAGAAGCCAGGACAGGCCCCTGTACTTGTCATCTATGGTAAAAAC | |
| | | AACCGGCCCTCAGGGATCCCAGACCGATTCTCTGGCTCCAGCTCAGGAAAC | |
| | | ACAGCTTCCTTGACCATCACTGGGGCTCAGGCGGAAGATGAGGCTGACTAT | |
| | | TACTGTAACTCCCGGGACAGCAGTGGTAACCATCCAAGGGTATTCGGCGGA | |
| | | GGGACCAAGCTGACCGTCCTA | |
| A-026 | H-CDR1 | SGGYYWS | 251 |
| | H-CDR2 | YIYYSGSTYYNPSLKS | 252 |
| | H-CDR3 | WSLGTSNHGWFDP | 253 |
| | VH (aa) | QVQLQESGPGLVKPSQTLSLTCTVSGGSISSGGYYWSWIRQHPGKGLEWIG | 254 |
| | | YIYYSGSTYYNPSLKSRVTISVDTSKNQFSLKLSSVTAADTAVYYCASWSL | |
| | | GTSNHGWFDPWGQGTLVTVSS | |
| | L-CDR1 | SGSSSNIGSNTVN | 255 |
| | L-CDR2 | DNNNRPS | 256 |
| | L-CDR3 | QSYDSNLSGWV | 257 |
| | VL (aa) | QSVLTQPPSASGTPGQRVTISCSGSSSNIGSNTVNWYQQLPGTAPKLLISD | 258 |
| | | NNNRPSGVPDRFSGSKSGTSASLAITGLQAEDEADYYCQSYDSNLSGWVFG | |
| | | GGTKLTVL | |
| | VH | CAGGTGCAACTGCAGGAGTCGGGCCCAGGACTGGTGAAGCCTTCACAGACC | 259 |
| | (DNA) | CTGTCCCTCACCTGCACTGTCTCTGGTGGCTCCATCAGCAGTGGTGGTTAC | |
| | | TACTGGAGCTGGATCCGCCAGCACCCAGGGAAGGGCCTGGAGTGGATTGGG | |
| | | TACATCTATTACAGTGGGAGCACCTACTACAACCCGTCCCTCAAGAGTCGA | |
| | | GTTACCATATCAGTAGACACGTCTAAGAACCAGTTCTCCCTGAAGCTGAGC | |
| | | TCTGTGACCGCCGCAGACACGGCTGTGTATTACTGTGCGAGCTGGTCCCTA | |
| | | GGTACCAGCAACCATGGTTGGTTCGACCCCTGGGGCCAGGGAACCCTGGTC | |
| | | ACCGTCTCCTCA | |
| | VL | CAGTCTGTGCTGACTCAGCCACCCTCAGCGTCTGGGACCCCCGGGCAGAGG | 260 |
| | (DNA) | GTCACCATCTCTTGTTCTGGAAGCAGCTCCAACATCGGAAGTAATACTGTA | |
| | | AACTGGTACCAGCAGCTTCCAGGAACAGCCCCCAAACTCCTCATCTCTGAT | |
| | | AATAACAATCGGCCCTCAGGGGTCCCTGACCGATTCTCTGGCTCCAAGTCT | |
| | | GGCACCTCAGCCTCCCTGGCCATCACTGGGCTCCAGGCTGAGGATGAGGCT | |
| | | GATTATTACTGCCAGTCCTATGACAGTAACCTGAGTGGTTGGGTGTTCGGC | |
| | | GGAGGGACCAAGCTGACCGTCCTA | |
| A-027 | H-CDR1 | SYAIS | 261 |
| | H-CDR2 | GIIPIFGTANYAQKFQG | 262 |
| | H-CDR3 | ARGSTWGYFDY | 263 |
| | VH (aa) | QVQLVQSGAEVKKPGSSVKVSCKASGGTFSSYAISWVRQAPGQGLEWMGGI | 264 |
| | | IPIFGTANYAQKFQGRVTITADKSTSTAYMELSSLRSEDTAVYYCARARGS | |
| | | TWGYFDYWGQGTLVTVSS | |
| | L-CDR1 | RASQSVSNYLA | 265 |
| | L-CDR2 | DASNRAT | 266 |
| | L-CDR3 | QQRDNWPLT | 267 |
| | VL (aa) | EIVLTQSPASLSLSPGERVTLSCRASQSVSNYLAWYQQKPGQAPRLLIYDA | 268 |
| | | SNRATGIPARFSGSGSGTDFTLTISSLEPEDFAVYYCQQRDNWPLTFGGGT | |
| | | KVEIK | |
| | VH | CAGGTGCAGCTGGTACAATCTGGGGCTGAGGTGAAGAAGCCTGGGTCCTCG | 269 |
| | (DNA) | GTGAAGGTCTCCTGCAAGGCTTCTGGAGGCACCTTCAGCAGCTATGCTATC | |
| | | AGCTGGGTGCGACAGGCCCCTGGACAAGGGCTTGAGTGGATGGGAGGGATC | |
| | | ATCCCTATCTTTGGTACAGCAAACTACGCACAGAAGTTCCAGGGCAGAGTC | |
| | | ACGATTACCGCGGACAAATCCACGAGCACAGCCTACATGGAGCTGAGCAGC | |
| | | CTGAGATCTGAGGACACGGCCGTGTATTACTGTGCGAGAGCGAGGGGCAGC | |
| | | ACCTGGGGCTACTTTGACTACTGGGGCCAGGGAACCCTGGTCACCGTCTCC | |
| | | TCA | |
| | VL | GAAATAGTGTTGACGCAGTCTCCAGCCTCCCTGTCTTTGTCTCCAGGGGAA | 270 |
| | (DNA) | AGAGTCACCCTCTCCTGCAGGGCCAGTCAGAGTGTTAGCAATTACTTAGCC | |
| | | TGGTATCAACAGAAACCTGGCCAGGCTCCCAGGCTCCTCATCTATGATGCA | |
| | | TCCAACAGGGCCACTGGCATCCCAGCCAGGTTCAGTGGCAGTGGGTCTGGG | |
| | | ACAGACTTCACTCTCACCATCAGCAGCCTAGAGCCCGAAGATTTTGCAGTT | |
| | | TATTACTGTCAGCAGCGTGACAACTGGCCCCTCACTTTCGGCGGAGGGACC | |
| | | AAGGTGGAGATCAAA | |
| A-028 | H-CDR1 | SYAIS | 271 |
| | H-CDR2 | GIIPIFGTANYAQKFQG | 272 |
| | H-CDR3 | VGVEYQLLWYFDY | 273 |
| | VH (aa) | QVQLVQSGAEVKKPGSSVKVSCKASGGTFSSYAISWVRQAPGQGLEWMGGI | 274 |
| | | IPIFGTANYAQKFQGRVTITADESTSTAYMELSSLRSEDTAVYYCARVGVE | |
| | | YQLLWYFDYWGQGTLVTVSS | |
| | L-CDR1 | RTSQTISNYLN | 275 |
| | L-CDR2 | AASNLQS | 276 |
| | L-CDR3 | QQSYNAS | 277 |
| | VL (aa) | DIQMTQSPSSLSASVGDRVTLTCRTSQTISNYLNWYQQKPGKAPKLLVYAA | 278 |
| | | SNLQSWVPSRFSGSGSGTDFTLTISSLQPEDFATYYCQQSYNASFGGGTKV | |
| | | EFK | |
| | VH | CAAGTGCAGCTGGTACAGTCTGGGGCTGAGGTGAAGAAGCCTGGGTCCTCG | 279 |
| | (DNA) | GTGAAGGTCTCCTGCAAGGCTTCTGGAGGCACCTTCAGCAGCTATGCAATC | |
| | | AGCTGGGTGCGACAGGCCCCTGGACAAGGGCTTGAGTGGATGGGAGGGATC | |
| | | ATCCCTATCTTTGGTACAGCAAACTACGCACAGAAGTTCCAGGGCAGAGTC | |
| | | ACGATTACCGCGGACGAATCCACGAGCACAGCCTACATGGAGCTGAGCAGC | |
| | | CTGAGATCTGAGGACACGGCCGTGTATTACTGTGCGAGAGTAGGGGTTGAG | |
| | | TACCAGCTGCTATGGTACTTTGACTACTGGGGCCAGGGAACCCTAGTCACC | |
| | | GTCTCCTCA | |
| | VL | GACATCCAGATGACCCAGTCTCCATCCTCCCTGTCTGCATCTGTAGGAGAC | 280 |
| | (DNA) | AGAGTCACCCTCACTTGCCGGACAAGTCAGACCATTAGCAACTATTTAAAT | |
| | | TGGTATCAGCAGAAACCAGGGAAAGCCCCTAAGCTCCTGGTCTATGCTGCA | |
| | | TCCAATTTGCAAAGTTGGGTCCCATCAAGGTTCAGTGGCAGTGGGTCTGGG | |
| | | ACAGATTTCACTCTCACCATCAGCAGTCTGCAGCCTGAAGATTTTGCAACT | |
| | | TACTACTGTCAACAGAGTTACAATGCCTCTTTCGGCGGAGGGACCAAGGTG | |
| | | GAGTTCAAA | |
| A-029 | H-CDR1 | SYAIS | 281 |
| | H-CDR2 | GIIPIFGTANYAQKFQG | 282 |
| | H-CDR3 | SYYYYYGMDV | 283 |
| | VH (aa) | QVQLVQSGAEVKKPGSSVKVSCKASGGTFSSYAISWVRQAPGQGLEWMGGI | 284 |
| | | IPIFGTANYAQKFQGRVTITADESTGTAYMELSSLRSEDTAVYYCARSYYY | |
| | | YYGMDVWGQGTTVTVSS | |
| | L-CDR1 | RSSQSLLHSNGYNYLD | 285 |
| | L-CDR2 | LGSNRAS | 286 |
| | L-CDR3 | MQAVDTPRT | 287 |
| | VL (aa) | DIQMTQSPLSLPVTPGEPASISCRSSQSLLHSNGYNYLDWYLQKPGQSPQL | 288 |
| | | LIHLGSNRASGVPDRFSGSGSGTDFTLKISRVEAEDVGVYYCMQAVDTPRT | |
| | | FGQGTKVDIK | |
| | VH | CAGGTCCAGCTGGTGCAGTCTGGGGCTGAGGTGAAGAAGCCTGGGTCCTCG | 289 |
| | (DNA) | GTGAAGGTCTCCTGCAAGGCTTCTGGAGGCACCTTCAGCAGCTATGCTATC | |
| | | AGCTGGGTGCGACAGGCCCCTGGACAAGGGCTTGAGTGGATGGGAGGGATC | |
| | | ATCCCTATCTTTGGTACAGCAAACTACGCACAGAAGTTCCAGGGCAGAGTC | |
| | | ACGATTACCGCGGACGAATCCACGGGCACAGCCTACATGGAGCTGAGCAGC | |
| | | CTGAGATCTGAGGACACGGCCGTGTATTACTGTGCGAGAAGTTACTACTAC | |
| | | TACTACGGTATGGACGTCTGGGGCCAAGGGACCACGGTCACCGTCTCCTCA | |
| | VL | GACATCCAGATGACCCAGTCTCCACTCTCCCTGCCCGTCACCCCTGGAGAG | 290 |
| | (DNA) | CCGGCCTCCATCTCCTGCAGGTCTAGTCAGAGCCTCCTGCATAGTAATGGA | |
| | | TACAACTATTTGGATTGGTACCTGCAGAAGCCAGGGCAGTCACCACAGCTC | |
| | | CTGATCCATTTGGGTTCTAATCGGGCCTCCGGGGTCCCTGACAGGTTCAGT | |
| | | GGCAGTGGATCAGGCACAGATTTTACACTGAAAATTAGCAGAGTGGAGGCT | |
| | | GAGGATGTTGGGGTTTATTACTGCATGCAAGCTGTAGATACTCCTCGGACG | |
| | | TTCGGCCAAGGGACCAAGGTGGACATCAAA | |
| A-030 | H-CDR1 | GYYMH | 291 |
| | H-CDR2 | WINPNSGNTGYAQKFQG | 292 |
| | H-CDR3 | GGQQQLVLDDY | 293 |
| | VH (aa) | QVQLVQSGAEVKKPGASVKVSCKASGYTFTGYYMHWVRQAPGQGLEWMGW1 | 294 |
| | | NPNSGNTGYAQKFQGRVTMTRNTSISTAYMELSSLRSEDTAVYYCARGGQQ | |
| | | QLVLDDYWGQGTLVTVSS | |
| | L-CDR1 | RASQSIGGWLA | 295 |
| | L-CDR2 | AASSLQS | 296 |
| | L-CDR3 | RQSYSTPPT | 297 |
| | VL (aa) | DIQLTQSPSTLSASVGDRVTITCRASQSIGGWLAWYQHKPGKAPKLLIYAA | 298 |
| | | SSLQSGVPSRFSGSGSGTDFTLTISSLQPEDFATYYCRQSYSTPPTFGQGT | |
| | | KVEIK | |
| | VH | CAGGTGCAGCTGGTGCAGTCTGGGGCTGAGGTGAAGAAGCCTGGGGCCTCA | 299 |
| | (DNA) | GTGAAGGTCTCCTGCAAGGCTTCTGGATACACCTTCACCGGCTACTATATG | |
| | | CACTGGGTGCGACAGGCCCCTGGACAAGGGCTTGAGTGGATGGGATGGATC | |
| | | AACCCTAACAGTGGTAACACAGGCTATGCACAGAAGTTCCAGGGCAGAGTC | |
| | | ACCATGACCAGGAACACCTCCATAAGCACAGCCTACATGGAGCTGAGCAGC | |
| | | CTGAGATCTGAGGACACGGCCGTGTATTACTGTGCGAGAGGCGGACAGCAG | |
| | | CAGCTGGTACTGGACGACTACTGGGGCCAGGGAACCCTGGTCACCGTCTCC | |
| | | TCA | |
| | VL | GACATCCAGTTGACCCAGTCTCCTTCCACCCTGTCAGCATCTGTAGGCGAC | 300 |
| | (DNA) | AGAGTCACCATCACTTGCCGGGCCAGTCAGTCTATTGGTGGTTGGTTGGCC | |
| | | TGGTATCAGCACAAACCAGGGAAAGCCCCCAAGCTCCTGATCTATGCTGCA | |
| | | TCCAGTTTGCAAAGTGGGGTCCCATCAAGGTTCAGTGGCAGTGGATCTGGG | |
| | | ACAGATTTCACTCTCACCATCAGCAGTCTGCAACCTGAAGATTTTGCAACT | |
| | | TACTACTGTCGACAGAGTTACAGTACCCCTCCGACGTTCGGCCAAGGGACC | |
| | | AAGGTGGAAATCAAA | |
| A-031 | H-CDR1 | SYAMH | 301 |
| | H-CDR2 | VISYDGSNKYYADSVKG | 302 |
| | H-CDR3 | VVAAADLTRYFDY | 303 |
| | VH (aa) | QVQLVESGGGVVQPGRSLRLSCAASGFTFSSYAMHWVRQAPGKGLEWVAVI | 304 |
| | | SYDGSNKYYADSVKGRFTISRDNSKNTLYLQMNSLRAEDTAVYYCARVVAA | |
| | | ADLTRYFDYWGQGTLVTVSS | |
| | L-CDR1 | RASQSVPKNYLA | 305 |
| | L-CDR2 | TASSRAP | 306 |
| | L-CDR3 | QQYGTSPNT | 307 |
| | VL (aa) | EIVLTQSPGTVSLSPGERATLSCRASQSVPKNYLAWFQQKPGQAPRLVIHT | 308 |
| | | ASSRAPGIPDRFTGSGSGTDFTLTISRLEPEDFAVYYCQQYGTSPNTFGQG | |
| | | TKLDIK | |
| | VH | CAGGTGCAGCTGGTGGAGTCTGGGGGAGGCGTGGTCCAGCCTGGGAGGTCC | 309 |
| | (DNA) | CTGAGACTCTCCTGTGCAGCCTCTGGATTCACCTTCAGTAGCTATGCTATG | |
| | | CACTGGGTCCGCCAGGCTCCAGGCAAGGGGCTGGAGTGGGTGGCAGTTATA | |
| | | TCATATGATGGAAGCAATAAATACTACGCAGACTCCGTGAAGGGCCGATTC | |
| | | ACCATCTCCAGAGACAATTCCAAGAACACGCTGTATCTGCAAATGAACAGC | |
| | | CTGAGAGCTGAGGACACGGCTGTGTATTACTGTGCGAGAGTCGTAGCAGCA | |
| | | GCTGACCTCACTCGCTACTTTGACTACTGGGGCCAGGGAACCCTGGTCACC | |
| | | GTCTCCTCA | |
| | VL | GAAATTGTGTTGACGCAGTCTCCAGGCACCGTGTCTTTGTCTCCAGGGGAA | 310 |
| | (DNA) | AGAGCCACCCTCTCCTGCAGGGCCAGTCAGAGTGTTCCTAAGAACTACTTA | |
| | | GCCTGGTTCCAGCAGAAACCTGGCCAGGCTCCCAGGCTCGTCATCCATACT | |
| | | GCATCCAGCAGGGCCCCTGGCATCCCAGACAGGTTCACTGGCAGCGGGTCT | |
| | | GGGACAGACTTCACTCTTACCATCAGCAGACTGGAGCCTGAAGATTTTGCA | |
| | | GTATATTACTGTCAGCAGTATGGCACCTCACCAAACACTTTTGGCCAGGGG | |
| | | ACCAAGCTGGACATCAAA | |
| A-032 | H-CDR1 | GYYMH | 311 |
| | H-CDR2 | WMNPNSGNTGYAQKFQG | 312 |
| | H-CDR3 | GGQQQLVLDDY | 313 |
| | VH (aa) | QVQLVQSGAEVKKPGASVKVSCKASGYTFTGYYMHWVRQAPGQGLEWMGWM | 314 |
| | | NPNSGNTGYAQKFQGRVTMTRNTSISTAYMELSSLRSEDTAVYYCARGGQQ | |
| | | QLVLDDYWGQGTLVTVSS | |
| | L-CDR1 | RSSQSLLHSNGYNYLD | 315 |
| | L-CDR2 | LGSNRAS | 316 |
| | L-CDR3 | MQALQTPRT | 317 |
| | VL (aa) | DIVMTQSPLSLPVTPGEPASISCRSSQSLLHSNGYNYLDWYLQKPGQSPQL | 318 |
| | | LIYLGSNRASGVPDRFSGSGSGTDFTLKISRVEAEDVGVYYCMQALQTPRT | |
| | | FGPGTKVDIK | |
| | VH | CAAGTGCAGCTGGTACAGTCTGGGGCTGAGGTGAAGAAGCCTGGGGCCTCA | 319 |
| | (DNA) | GTGAAGGTCTCCTGCAAGGCTTCTGGATACACCTTCACCGGCTACTATATG | |
| | | CACTGGGTGCGACAGGCCCCTGGACAAGGGCTTGAGTGGATGGGGTGGATG | |
| | | AATCCTAACAGTGGCAACACAGGCTATGCACAGAAGTTCCAGGGCAGAGTC | |
| | | ACCATGACCAGGAACACCTCCATAAGCACAGCCTACATGGAGCTGAGCAGC | |
| | | CTGAGATCTGAGGACACGGCCGTGTATTACTGTGCGAGAGGCGGACAGCAG | |
| | | CAGCTGGTACTGGACGACTACTGGGGCCAGGGAACCCTGGTCACCGTCTCC | |
| | | TCA | |
| | VL | GACATCGTGATGACCCAGTCTCCACTCTCCCTGCCCGTCACCCCTGGAGAG | 320 |
| | (DNA) | CCGGCCTCCATCTCCTGCAGGTCTAGTCAGAGCCTCCTGCATAGTAATGGA | |
| | | TACAACTATTTGGATTGGTACCTGCAGAAGCCAGGGCAGTCTCCACAGCTC | |
| | | CTGATCTATTTGGGTTCTAATCGGGCCTCCGGGGTCCCTGACAGGTTCAGT | |
| | | GGCAGTGGATCAGGCACAGATTTTACACTGAAAATCAGCAGAGTGGAGGCT | |
| | | GAGGATGTTGGGGTTTATTACTGCATGCAAGCTCTACAAACCCCTCGGACT | |
| | | TTCGGCCCTGGGACCAAAGTGGATATCAAA | |
| A-033 | H-CDR1 | SYGIS | 321 |
| | H-CDR2 | WISAYNGNTNYAQKLQG | 322 |
| | H-CDR3 | DHSIVGATTFDY | 323 |
| | VH (aa) | QVQLVQSGAEVKKPGASVKVSCKASGYTFTSYGISWVRQAPGQGLEWMGWI | 324 |
| | | SAYNGNTNYAQKLQGRVTMTTDTSTSTAYMELRSLRSDDTAVYYCARDHSI | |
| | | VGATTFDYWGQGTLVTVSS | |
| | L-CDR1 | RASQSISSWLA | 325 |
| | L-CDR2 | DASSLES | 326 |
| | L-CDR3 | QQYNSYPWT | 327 |
| | VL (aa) | DIVMTQSPSTLSASVGDRVTITCRASQSISSWLAWYQQKPGKAPKLLIYDA | 328 |
| | | SSLESGVPSRFSGSGSGTEFTLTISSLQPDDFATYYCQQYNSYPWTFGQGT | |
| | | KVEIK | |
| | VH | CAGGTCCAGCTGGTGCAGTCTGGAGCTGAGGTGAAGAAGCCTGGGGCCTCA | 329 |
| | (DNA) | GTGAAGGTCTCCTGCAAGGCTTCTGGTTACACCTTTACCAGCTACGGTATC | |
| | | AGCTGGGTGCGACAGGCCCCTGGACAAGGGCTTGAGTGGATGGGATGGATC | |
| | | AGCGCTTACAATGGTAACACAAACTATGCACAGAAGCTCCAGGGCAGAGTC | |
| | | ACCATGACCACAGACACATCCACGAGCACAGCCTACATGGAGCTGAGGAGC | |
| | | CTGAGATCTGACGACACGGCCGTGTATTACTGTGCGAGAGATCACAGTATA | |
| | | GTGGGAGCTACTACGTTTGACTACTGGGGCCAGGGAACCCTGGTCACCGTC | |
| | | TCCTCA | |
| | VL | GACATCGTGATGACCCAGTCTCCTTCCACCCTGTCTGCATCTGTAGGAGAC | 330 |
| | (DNA) | AGAGTCACCATCACTTGCCGGGCCAGTCAGAGTATTAGTAGCTGGTTGGCC | |
| | | TGGTATCAGCAGAAACCAGGGAAAGCCCCTAAGCTCCTGATCTATGATGCC | |
| | | TCCAGTTTGGAAAGTGGGGTCCCATCAAGGTTCAGCGGCAGTGGATCTGGG | |
| | | ACAGAATTCACTCTCACCATCAGCAGCCTGCAGCCTGATGATTTTGCAACT | |
| | | TATTACTGCCAACAGTACAATAGTTATCCGTGGACGTTCGGCCAAGGGACC | |
| | | AAGGTGGAAATCAAA | |
| A-034 | H-CDR1 | SYTMN | 331 |
| | H-CDR2 | SISSIGTYIYYADSVKG | 332 |
| | H-CDR3 | VLLSGSYYGYFDS | 333 |
| | VH (aa) | QMQLVQSGGGLVKPGGSLRLSCAASGFTFSSYTMNWVRQAPGKGLEWVSSI | 334 |
| | | SSIGTYIYYADSVKGRFTISRDNAKNSLYLQMNSLRAEDTAVYYCARVLLS | |
| | | GSYYGYFDSWGQGTLVTVSS | |
| | L-CDR1 | TGSSSNIGAGYDVH | 335 |
| | L-CDR2 | GNSNRPS | 336 |
| | L-CDR3 | QSYDSSLSGYV | 337 |
| | VL (aa) | QSVLTQPPSVSGAPGQRVTISCTGSSSNIGAGYDVHWYQQLPGTAPKLFIY | 338 |
| | | GNSNRPSGVPDRFSGSKSGTSASLAITGLQAEDEADYYCQSYDSSLSGYVF | |
| | | GTGTKVTVL | |
| | VH | CAGATGCAGCTGGTACAGTCTGGGGGAGGCCTGGTCAAGCCTGGGGGGTCC | 339 |
| | (DNA) | CTGAGACTCTCCTGTGCAGCCTCTGGATTCACCTTCAGTAGCTATACCATG | |
| | | AACTGGGTCCGCCAGGCTCCAGGGAAGGGGCTGGAGTGGGTCTCATCCATT | |
| | | AGTAGTATTGGTACTTACATATACTACGCAGACTCAGTGAAGGGCCGATTC | |
| | | ACCATCTCCAGAGACAACGCCAAGAACTCACTGTATCTGCAAATGAACAGC | |
| | | CTGAGAGCCGAGGACACGGCTGTGTATTACTGTGCGAGAGTCCTGTTAAGT | |
| | | GGGAGCTACTACGGCTACTTTGACTCCTGGGGCCAGGGAACCCTGGTCACC | |
| | | GTCTCCTCA | |
| | VL | CAGTCTGTGCTGACTCAGCCACCCTCAGTGTCTGGGGCCCCAGGGCAGAGG | 340 |
| | (DNA) | GTCACCATCTCCTGCACTGGGAGCAGCTCCAACATCGGGGCAGGTTATGAT | |
| | | GTGCACTGGTACCAGCAGCTTCCAGGAACAGCCCCCAAACTCTTCATCTAT | |
| | | GGTAACAGCAATCGGCCCTCAGGGGTCCCTGACCGATTCTCTGGCTCCAAG | |
| | | TCTGGCACCTCAGCCTCCCTGGCCATCACTGGGCTCCAGGCTGAGGATGAG | |
| | | GCTGATTATTACTGCCAGTCCTATGACAGCAGCCTGAGTGGTTATGTCTTC | |
| | | GGAACTGGGACCAAGGTCACCGTCCTA | |
| A-035 | H-CDR1 | GYYMH | 341 |
| | H-CDR2 | WINPNSGNTGYAQKFQG | 342 |
| | H-CDR3 | GRLERGYWYFDL | 343 |
| | VH (aa) | QVQLVQSGAEVKKPGASVKVSCKASGYTFTGYYMHWVRQAPGQGLEWMGW1 | 344 |
| | | NPNSGNTGYAQKFQGRVTMTRNTSISTAYMELSSLRSEDTAVYYCARGRLE | |
| | | RGYWYFDLWGRGTLVTVSS | |
| | L-CDR1 | TRSSGSITSNYVQ | 345 |
| | L-CDR2 | EDKERPS | 346 |
| | L-CDR3 | QSYGGTSQGVL | 347 |
| | VL (aa) | NFMLTQPHSVSESPGRTVTISCTRSSGSITSNYVQWYQQRPGSAPTILIYE | 348 |
| | | DKERPSEVPDRFSGSIDISSNSASLTISGLKTEDEADYYCQSYGGTSQGVL | |
| | | FGGGTKVTVL | |
| | VH | CAGGTCCAGCTGGTGCAGTCTGGGGCTGAGGTGAAGAAGCCTGGGGCCTCA | 349 |
| | (DNA) | GTGAAGGTCTCCTGCAAGGCTTCTGGATACACCTTCACCGGCTACTATATG | |
| | | CACTGGGTGCGACAGGCCCCTGGACAAGGGCTTGAGTGGATGGGATGGATC | |
| | | AACCCTAACAGTGGTAACACAGGCTATGCACAGAAGTTCCAGGGCAGAGTC | |
| | | ACCATGACCAGGAACACCTCCATAAGCACAGCCTACATGGAGCTGAGCAGC | |
| | | CTGAGATCTGAGGACACGGCCGTGTATTACTGTGCGAGAGGCCGTCTGGAA | |
| | | CGCGGATACTGGTACTTCGATCTCTGGGGCCGTGGCACCCTGGTCACCGTC | |
| | | TCCTCA | |
| | VL | AATTTTATGCTGACTCAGCCCCACTCTGTGTCGGAGTCTCCGGGGAGGACG | 350 |
| | (DNA) | GTAACCATCTCCTGCACCCGCAGCAGTGGCAGCATTACCAGCAACTATGTC | |
| | | CAGTGGTACCAGCAGCGCCCGGGCAGTGCCCCTACCATCCTAATCTATGAG | |
| | | GATAAGGAAAGACCCTCTGAGGTCCCTGATCGCTTCTCTGGCTCCATCGAC | |
| | | ATTTCCTCCAACTCTGCCTCCCTCACCATCTCTGGCCTGAAGACGGAGGAC | |
| | | GAGGCTGACTACTACTGTCAGTCTTATGGTGGCACCAGTCAAGGGGTGTTA | |
| | | TTCGGCGGAGGGACCAAGGTGACCGTCCTA | |
| A-036 | H-CDR1 | GYYMH | 351 |
| | H-CDR2 | WINPNSGGTNYAQKFQG | 352 |
| | H-CDR3 | GGQQQLVLDDY | 353 |
| | VH (aa) | QVQLVQSGAEVKKPGASVKVSGKASGYTFTGYYMHWVRQAPGQGLEWMGW1 | 354 |
| | | NPNSGGTNYAQKFQGRVTMTRNTSISTAYMELSSLRSEDTAVYYCARGGQQ | |
| | | QLVLDDYWGQGTLVTVSS | |
| | L-CDR1 | RASQSISSYLN | 355 |
| | L-CDR2 | AASSLQS | 356 |
| | L-CDR3 | QQSYSTPRT | 357 |
| | VL (aa) | DIVMTQSPSSLSASVGDRVTITCRASQSISSYLNWYQQKPGKAPKLLIYAA | 358 |
| | | SSLQSGVPSRFSGSGSGTDFTLTISSLQPEDFATYYCQQSYSTPRTFGQGT | |
| | | KVEIK | |
| | VH | CAGGTGCAGCTGGTGCAATCTGGGGCTGAGGTGAAGAAGCCTGGGGCCTCA | 359 |
| | (DNA) | GTGAAGGTCTCCTGCAAGGCTTCTGGATACACCTTCACCGGCTACTATATG | |
| | | CACTGGGTGCGACAGGCCCCTGGACAAGGGCTTGAGTGGATGGGATGGATC | |
| | | AACCCTAACAGTGGTGGCACAAACTATGCACAGAAGTTCCAGGGCAGAGTC | |
| | | ACCATGACCAGGAACACCTCCATAAGCACAGCCTACATGGAGCTGAGCAGC | |
| | | CTGAGATCTGAGGACACGGCCGTGTATTACTGTGCGAGAGGCGGACAGCAG | |
| | | CAGCTGGTACTGGACGACTACTGGGGCCAGGGAACCCTGGTCACCGTCTCC | |
| | | TCA | |
| | VL | GACATCGTGATGACCCAGTCTCCATCCTCCCTGTCTGCATCTGTAGGAGAC | 360 |
| | (DNA) | AGAGTCACCATCACTTGCCGGGCAAGTCAGAGCATTAGCAGCTATTTAAAT | |
| | | TGGTATCAGCAGAAACCAGGGAAAGCCCCTAAGCTCCTGATCTATGCTGCA | |
| | | TCCAGTTTGCAAAGTGGGGTCCCATCAAGGTTCAGTGGCAGTGGATCTGGG | |
| | | ACAGATTTCACTCTCACCATCAGCAGTCTGCAACCTGAAGATTTTGCAACT | |
| | | TACTACTGTCAACAGAGTTACAGTACCCCTCGAACGTTCGGCCAAGGGACC | |
| | | AAGGTGGAAATCAAA | |
| A-037 | H-CDR1 | GYYMH | 361 |
| | H-CDR2 | WINPNSGNTGYAQKFQG | 362 |
| | H-CDR3 | GGQQQLVLDDY | 363 |
| | VH (aa) | QVQLVQSGAEVKKPGASVKVSCKASGYTFTGYYMHWVRQAPGQGLEWMGW1 | 364 |
| | | NPNSGNTGYAQKFQGRVTMTRNTSISTAYMELSSLRSEDTAVYYCARGGQQ | |
| | | QLVLDDYWGQGTLVTVSS | |
| | L-CDR1 | TGSSGSIASNYVQ | 365 |
| | L-CDR2 | EDNQRPS | 366 |
| | L-CDR3 | QSYDSSNQRV | 367 |
| | VL (aa) | NFMLTQPHSVSESPGKTVTISCTGSSGSIASNYVQWYQQRPGSAPTTVIYE | 368 |
| | | DNQRPSGVPDRFSGSIDSSSNSASLTISGLKTEDEADYYCQSYDSSNQRVF | |
| | | GGGTKLTVL | |
| | VH | CAGGTTCAGCTGGTGCAGTCTGGGGCTGAGGTGAAGAAGCCTGGGGCCTCA | 369 |
| | (DNA) | GTGAAGGTCTCCTGCAAGGCTTCTGGATACACCTTCACCGGCTACTATATG | |
| | | CACTGGGTGCGACAGGCCCCTGGACAAGGGCTTGAGTGGATGGGATGGATC | |
| | | AACCCTAACAGTGGTAACACAGGCTATGCACAGAAATTCCAGGGCAGAGTC | |
| | | ACCATGACCAGGAACACCTCCATAAGCACAGCCTACATGGAGCTGAGCAGC | |
| | | CTGAGATCTGAGGACACGGCCGTGTATTACTGTGCGAGAGGCGGACAGCAG | |
| | | CAGCTGGTACTGGACGACTACTGGGGCCAGGGAACCCTGGTCACCGTCTCC | |
| | | TCA | |
| | VL | AATTTTATGCTGACTCAGCCCCACTCTGTGTCGGAGTCTCCGGGGAAGACG | 370 |
| | (DNA) | GTAACCATCTCCTGCACCGGCAGCAGTGGCAGCATTGCCAGCAACTATGTG | |
| | | CAGTGGTACCAGCAGCGCCCGGGCAGTGCCCCCACCACTGTGATCTATGAG | |
| | | GATAACCAAAGACCCTCTGGGGTCCCTGATCGGTTCTCTGGCTCCATCGAC | |
| | | AGCTCCTCCAACTCTGCCTCCCTCACCATCTCTGGACTGAAGACTGAGGAC | |
| | | GAGGCTGACTACTACTGTCAGTCTTATGATAGCAGCAATCAGAGGGTGTTC | |
| | | GGCGGAGGGACCAAGCTGACCGTCCTA | |

TABLE IB
Germline of VH and VL for ABPs of Comparative Example 3.

| Antibody | Germline of VH | Germline of VL |
|---|---|---|
| A-001 | IGHV3-21*03 | IGKV1-39*01 |
| A-002 | IGHV3-53*01 | IGLV7-43*01 |
| A-003 | IGHV1-69D*01 | IGLV2-14*01 |
| A-004 | IGHV3-21*03 | IGKV1-39*01 |
| A-005 | IGHV3-53*01 | IGLV7-43*01 |
| A-006 | IGHV3-53*01 | IGLV7-43*01 |
| A-007 | IGHV1-2*02 | IGLV2-14*01 |
| A-008 | IGHV4-39*07 | IGLV1-40*01 |
| A-009 | IGHV1-69D*01 | IGKV3D-15*01 |
| A-010 | IGHV1-18*04 | IGLV2-14*01 |
| A-011 | IGHV3-30*11 | IGLV3-19*01 |
| A-012 | IGHV3-53*01 | IGLV7-43*01 |
| A-013 | IGHV3-53*01 | IGLV7-43*01 |
| A-014 | IGHV1-2*02 | IGKV4-l*01 |
| A-015 | IGHV1-2*02 | IGLV4-2*01 |
| A-016 | IGHV1-69D*01 | IGLV2-14*01 |
| A-017 | IGHV1-2*02 | IGKV1D-33*01 |
| A-018 | IGHV3-30*11 | IGKV3-20*01 |
| A-019 | IGHV1-2*02 | IGKV3D-15*01 |
| A-020 | IGHV4-31*02 | IGLV2-8*01 |
| A-021 | IGHV1-18*04 | IGLV2-14*01 |
| A-022 | IGHV4-31*02 | IGLV10-54*01 |
| A-023 | IGHV1-69D*01 | IGLV1-51*01 |
| A-024 | IGHV1-18*04 | IGLV2-14*01 |
| A-025 | IGHV1-69D*01 | IGLV3-19*01 |
| A-026 | IGHV4-31*02 | IGLV1-44*01 |
| A-027 | IGHV1-69*14 | IGKV3-11*01 |
| A-028 | IGHV1-69D*01 | IGKV1-39*01 |
| A-029 | IGHV1-69D*01 | IGKV2-28*01 |
| A-030 | IGHV1-2*02 | IGKV1-39*01 |
| A-031 | IGHV3-30*11 | IGKV3-20*01 |
| A-032 | IGHV1-8*01 | IGKV2-28*01 |
| A-033 | IGHV1-18*04 | IGKV1-5*01 |
| A-034 | IGHV3-21*03 | IGLV1-40*01 |
| A-035 | IGHV1-2*02 | IGLV6-57*02 |
| A-036 | IGHV1-2*02 | IGKV1-39*01 |
| A-037 | IGHV1-2*02 | IGLV6-57*02 |

TABLE 1B.1
Germline of VH and VL for further
ABPs of Comparative Example 3.

| | | |
|---|---|---|
| B-001 | IGHV3-53*01 | IGLV7-43*01 |
| B-002 | IGHV3-53*01 | IGLV7-43*01 |
| B-003 | IGHV3-53*01 | IGLV7-43*01 |
| B-004 | IGHV3-53*01 | IGLV7-43*01 |
| B-005 | IGHV3-53*01 | IGLV7-43*01 |
| B-006 | IGHV3-53*01 | IGLV7-43*01 |
| B-007 | IGHV3-53*01 | IGLV7-43*01 |
| B-008 | IGHV3-53*01 | IGLV7-43*01 |

TABLE 2
Binding of ABPs of Comparative Example 3 to various antigens.

| | Human | Strept- | Mouse | Mouse | Cell | |
|---|---|---|---|---|---|---|
| Antibody | IGSF11 | avidin | IGSF11 | Fc | binding | MFI |
| A-001 | *** | − | *** | − | + + + | ND |
| A-002 | ** | − | * | − | + | ND |
| A-003 | * | − | * | − | + | ND |
| A-004 | ** | − | *** | − | + + + | ND |
| A-005 | ** | − | * | − | + + | ND |
| A-006 | ** | − | ** | − | + + | ND |
| A-007 | ** | − | ** | − | + | ND |
| A-008 | *** | − | *** | − | + + | ND |
| A-009 | ** | − | * | − | + + | ND |
| A-010 | ** | − | ** | − | + + | ND |
| A-011 | * | − | *** | − | + + | ND |
| A-012 | ** | − | ** | − | + | ND |
| A-013 | ** | − | ** | − | + | ND |
| A-014 | ** | − | * | − | + + | ND |
| A-015 | ** | − | ** | − | + + + | ND |
| A-016 | *** | − | ** | − | + + + | ND |
| A-017 | ** | − | * | − | + + | ND |
| A-018 | ** | − | ** | − | + + | ND |
| A-019 | *** | − | ** | − | + + | ND |
| A-020 | *** | − | ** | − | + + + | ### |
| A-021 | ** | − | * | − | + + + | ### |
| A-022 | ** | − | *** | − | + + | ## |
| A-023 | ** | − | * | − | + + + | ### |
| A-024 | ** | − | * | − | + + + | ### |
| A-025 | * | − | * | − | + + + | ## |
| A-026 | *** | − | *** | − | + + | ## |
| A-027 | * | − | * | − | + + | ## |
| A-028 | * | − | * | − | + + + | ### |
| A-029 | * | − | * | − | + + | ## |
| A-030 | * | − | * | − | + + | ## |
| A-031 | * | − | * | − | + + | ## |
| A-032 | * | − | * | − | + + | # |
| A-033 | * | − | * | − | + + | # |
| A-034 | *** | − | ** | − | + + | # |
| A-035 | ** | − | *** | − | + | # |
| A-036 | * | − | * | − | + | # |
| A-037 | * | − | * | − | + + | ## |

Binding (relative fluorescent units, RFU) indicated by:
″***″ = >1.0;
″**″ = approx 0.5 to 1.0;
″*″ = approx 0.1 to 0.5;
″—″ = <0.025;
″−″ = <0.05;
Cell binding (% positive c (ells in FACS) indicated by:
″+++″ = >50%;
″++″ = approx 35% to 50%;
″+″ = approx <25%;
mean fluorescent intensity (MFI) (RFU by FACS) indicated by:
″###″ = >150;
″##″ = approx 100 to 150;
″#″ = approx <100.

Alignments of sequences of the variable regions of antibodies of Comparative Example 3 demonstrated that amino acid substitutions are permitted, within either or both of the hypervariable CDR and/or the framework sequences of the variable regions; and indeed, indicated that amino acid insertions and/or deletions would also be permitted within the sequences of the variable regions disclosed herein (FIG. 10). Thus, antibodies having one or more variable regions comprising one or more amino acid substitutions, insertions and/or deletions compared to the variable domains disclosed herein, can also be considered antibodies of Comparative Example 3. Indeed, that amino acid deletions in the hypervariable CDR and/or the framework sequences of the variable regions can be permitted is further supported by the sequence of the VH domain of A-003 as follows. Based on its germline homology, such VH should begin with a “Q”, and re-sequencing of the original phage clone and the resulting scFv clone confirmed that the initial Q″ was indeed missing (compared to corresponding germline sequence). The original phage clone was found to bind to IGSF11, and the scFv produced following recloning (both missing the “Q”) was also found to bind to IGSF11.

### COMPARATIVE EXAMPLE 4: FUNCTIONAL CHARACTERISATION OF ABPS OF COMPARATIVE EXAMPLE 3 BY INHIBITION OF THE INTERACTION BETWEEN HUMAN IGSF11 (VSIG3) AND VSIR/VISTA)

Antibodies of Comparative Example 3 that bind to IGSF11 (VSIG3) are also found to function as inhibitors of the interaction between IGSF11 (VSIG3) and VSIR (VISTA), ie the binding of (eg a function and/or activity of) IGSF11 (VSIG3) to VSIR (VISTA).

An ELISA assay was established to measure the inhibition of the binding of IGSF11 (VSIG3) to VSIR (VISTA) by antibodies (eg of the invention) that bind to IGSF11 (VSIG3). FIG. 3 demonstrates that this assay can detect the inhibition of binding of VSIR (VISTA) by competition for binding to IGSF11 (VSIG3). Purified and immobilised ECD of human IGSF11 (VSIG3) (HIS6-tagged) can interact with VSIR (VISTA), and this interaction is detected, briefly as follows: recombinant, purified and biotinylated IGSF11 (VSIG3) is immobilised on a streptavidin-coated plate at 5 pg/mL (in PBS); purified Fc-tagged VSIR (VISTA) (R&D Systems, Cat #7126-B7) is added for binding (eg at 1.8 ug/mL; approx. 20 nM bivalent VSIR-FC), and after incubation for 1 hour at room temperature, unbound VSIR is removed by washing 3 times with PBS/Tween 0.05%; remaining VSIR bound to IGSF11 is detected using an appropriately labelled antibody against the Fc-tag (eg horseradish peroxidase-conjugated goat anti-human IgG, Jackson ImmunoResearch, Cat #115-036-098). This interaction can be blocked with soluble ECD of IGSF11, and as the interaction appears relatively weak (KD estimated by Yang et al (2017) to be at the level of 10e-5 M which belongs to low affinity protein—protein interactions between cell surface receptors) it can also easily be blocked by a commercial anti-VSIR (anti-VISTA) antibody (eg, R&D Systems, Cat #: MAB71261, monoclonal mouse IgG2b, or AF7126, polyclonal sheep).

IGSF11-binding antibodies of Comparative Example 3 from those shown in Tables 1 were tested in scFv-format for their ability to inhibit the interaction between IGSF11 (VSIG3) and with VSIR (VISTA) in this ELISA assay, and the degree of such inhibition is shown in Table 3. Briefly, E. coli-culture supernatant of scFv-producing phage-infected bacteria is added to surface-immobilised ECD of IGFS11 (VSIG3), and the unbound scFv washed away. The binding of VSIR (VISTA) to the scFv-treated IGSF11 is then assessed as described above

TABLE 3
Inhibition of the interaction between IGSF11 (VSIG3) and
with VSIR (VISTA) by IGSF11-binding antibodies
of Comparative Example 3 (ScFv format).

| | Binding |
|---|---|
| Antibody | inhibition |
| A-001 | — |
| A-002 | — |
| A-003 | * |
| A-004 | * |
| A-005 | — |
| A-006 | ** |
| A-007 | *** |
| A-008 | * |
| A-009 | * |
| A-010 | *** |
| A-011 | ** |
| A-012 | ** |
| A-013 | *** |
| A-014 | *** |
| A-015 | *** |
| A-016 | * |
| A-017 | — |
| A-018 | * |
| A-019 | * |

Inhibition of binding indicated by % or remaining bound VSIR as follows:
“***” = approx <55%;
“**” = approx 55% to 75%;
“*” = approx 75% to 95%;
“—” = >95%.

### COMPARATIVE EXAMPLE 5: FUNCTIONAL CHARACTERISATION OF SCFV-FC-FORMAT ABPS OF COMPARATIVE EXAMPLE 3

Antibodies of Comparative Example 3 in scFv-format are re-cloned, genetically fused to mouse Fc domain for mouse IgG2A and expressed in a HEK293 based expression system. The ability of such scFv-Fc-format ABPs of Comparative Example 3 to inhibit the interaction between IGSF11 (VSIG3) and VSIR (VISTA) can be tested in an ELISA-format assay as follows: (1) Recombinant purified human IGSF11 (VSIG3) ECD (HIS-tagged for purification) was immobilised on an ELISA plate (Nunc MaxiSorp) at 5 μg/mL (in PBS), and the plates were then washed and blocked with 2% BSA in PBS/Tween (0.05%); (2) A dilution series (10 ug/mL starting concentration, with a set of seven 5-fold dilutions) of anti-IGSF11 scFv-Fc (mouse IgG2A), or control scFv-Fc (mouse IgG2A) antibody of irrelevant specificity, was added for binding, and after plates were then washed of unbound antibody; (3) A dilution series (cross-dilution, 20 ug/mL starting concentration, with a set of seven 3-fold dilutions) of human VSIR-Fc (human IgG1) was added (R&D Systems, Cat #7126-B7), and after binding, unbound VSIR-Fc was removed by washing; (4) VSIR-Fc bound to immobilised IGSF11 was detected with a horseradish peroxidase-conjugated goat anti-human IgG (Fc specific, minimal species cross-reactive to mouse IgG2A of the IGSF11-Fc) (Jackson ImmunoResearch, Cat #115-036-098), and after washing the ELISA signal was developed with 3,3,5,5′-Tetramethylbenzidine (TMB) substrate. All binding steps were for 1 hour at room temperature, and all washing steps were three times washing with PBS/Tween (0.05%).

Indeed, at least one ABP of this Comparative Example is shown to inhibit the IGSF11-VSIR interaction with an IC50 of less than 1.5 nM in such an assay, where Fc-VSIR was added at a concentration of approximately 6.6 ug/mL (approximately 74 nM divalent Fc-VSIR concentration) (FIG. 4A). Indeed, the IC50 of this scFv-Fc-format ABP of the invention estimated in this assay, ranged from about 2.2 mM to 1.6 mM when Fc-VSIR was added at a concentration ranging from about 20 ug/mL to 0.75 ug/mL (about 222 nM to 8.2 nM dimer concentration), respectively (FIG. 4B).

Analogously, antibodies of Comparative Example 3 in scFv-format are re-cloned and expressed in a human IgG1-format. scFv-Fc-format and/or IgG1-format antibodies re-cloned from those in Table 3 are found to also inhibit the interaction between IGSF11 (VSIG3) and VSIR (VISTA) in the ELISA assay described in Example 4. Alternatively, the IgG1-format antibodies could be tested in an alternative ELISA set-up, where VSIR (VISTA) is immobilised, the antibody/ies for testing are bound to IGSF11 (VSIG3) in solution, the resulting complex is added to the immobilized VSIR (VISTA) and any residual binding of IGSF11 (VSIG3) (eg, His-tagged IGSF11 or IGSF11-Fc fusion protein) to VSIR (VISTA) is detected after washing to detect VSIR-bound IGSF11 using an appropriate anti-IGSF11 primary antibody (eg, anti-IGSF11 sheep polyclonal Ab cat #: AF4915 R&D Systems, or anti-His tag antibody) and a labelled secondary antibody suitable for the primary antibody. As in the set-up described above, reduced ELISA signals indicate antibodies of the Comparative Examples which inhibit the interaction between IGSF11 and VSIR (eg, a function and/or activity of IGSF11).

More specifically, the antibodies set forth in Table 5.1 are recloned from scFv-format into IgG1-format human, and are tested in ELISA assays for binding to IGSF11-HIS and IGSF11-hFc; briefly as described as follows: (1) antigen coating (IGSF11-HIS or IGSF11-hFc) at 2 ug/mL in PBS (o/n) and respective controls (blocking, streptavidin or counter antigens); (2) blocking with PBS+0.05% (v/v) Tween+2% (w/v) BSA; (3) plate washed 3× with PBS+0.05% (v/v) Tween; (4) addition of dilution series of respective antibody (in PBS+0.05% (v/v) Tween+2% (w/v) BSA) and incubation for 1h; (5) plate washed 3× with PBS+0.05% (v/v) Tween; (6) detection with either anti-human (Fc-specific) antibody-HRP conjugate or anti-human (Fab-specific) antibody-HRP conjugate diluted in PBS+0.05% (v/v) Tween+2% (w/v) BSA (1 h); (7) plate washed 3× with PBS+0.05% (v/v) Tween; and (8) ELISA developed with TMB substrate and reaction stopped after max 30 min with 1N HCl.

Furthermore, the binding affinities of IgG1-format antibodies of the Comparative Examples to IGSF11 are estimated using surface plasmon resonance (SPR; Biacore) and/or bio-layer interferometry (BLI; Octet) techniques.

IgG1-format antibodies of the Comparative Examples are also tested for their ability to inhibit binding between IGSF11 (VSIG3) and VSIR (VISTA) in the alternative binding assay as summarised above, and described in more detail as follows: (1) recombinant purified human VSIR-Fc (human IgG1) (R&D Systems, Cat #7126-B7) was immobilised on an ELISA plate (Nunc MaxiSorp) at 2 ug/mL (in PBS), and the plates were then washed and blocked with 2% BSA in PBS/Tween (0.050/); (2) a dilution series (500 nM starting concentration, with a set of nine 4-fold dilutions) of anti-IGSF11 IgG, or control IgG antibody of irrelevant specificity, was pre-incubated with 200 nM IGSF11 (VSIG3) ECD (his-tagged, SinoBiological, Cat #13094-H08H) for 30 minutes; (3) IGSF11-antibody complexes were added to the immobilised VSIR-Fc (human IgG1) for binding, and plates were then washed to remove unbound IGSF11 (VSIG3) ECD (his-tagged); (4) IGSF11 (VSIG3) ECD (his-tagged) bound to immobilized VSIR-Fc (human IgG1) was detected with a horseradish peroxidase-conjugated goat anti-hexahistidine antibody (Abcam, Cat #Ab1269), and after washing the ELISA signal was developed with 3,3,5,5′-Tetramethylbenzidine (TMB) substrate. All binding steps were for 1 hour at room temperature, and all washing steps were three times washing with PBS/Tween (0.05%).

Table 5.1 summarises the data obtained for IgG1-format antibodies of the Comparative Examples.

TABLE 5.1
Characterisation of antibodies of the Comparative
Examples to IGSF11 (VSIG3) inhibition of binding
between IGSF11 (VSIG3) and VSIR (VISTA)

| | Binding: | | | | |
|---|---|---|---|---|---|
| | HIS- | Binding: | Affinity: | Affinity: | Inhibition: |
| | IGSF11 | hFc-IGSF11 | SPR | BLI | IGSF11- |
| Antibody | EC50 (nM) | EC50 (nM) | Kd (nM) | Kd (nM) | VSIR |
| A-026 | *** | not | >500 | 20-30 | +++ |
| | | saturated at | | | |
| | | 100 nM | | | |
| A-004 | **** | | 100-250 | | − |
| A-013 | **** | ### | 100-250 | | +++ |
| A-012 | *** | ### | 250-500 | 1-10 | +++ |
| A-011 | **** | ## | 250-500 | 10-20 | +++ |
| A-006 | **** | *** | 250-500 | 1-10 | +++ |
| A-034 | ** | | 250-500 | | − |
| A-024 | *** | | >500 | n.d. | − |
| A-022 | **** | ### | n.d. | | +++ |
| A-035 | *** | # | n.d. | | ++ |
| A-015 | * | weak | | | + |
| | | binding | | | |
| A-007 | **** | ### | n.d. | | +++ |
| A-010 | **** | | | | − |
| A-014 | | weak | | | + |
| | | binding | | | |
| A-001 | **** | | | | − |
| A-018 | * | | | | n.d. |
| A-023 | ** | | | | − |
| A-025 | ** | | | | − |
| A-033 | *** | | | | − |
| A-031 | * | | | | n.d. |
| A-020 | *** | | | | − |
| A-027 | * | # | | | + + |
| A-003 | ** | | | | − |
| A-008 | *** | weak | | | + |
| | | binding | | | |
| A-009 | ** | | | | − |
| A-016 | **** | weak | | | + |
| | | binding | | | |
| A-028 | | | | | − |
| A-029 | ** | | | | − |
| A-037 | not | | | | − |
| | saturated | | | | |
| | at 100 nM | | | | |
| A-019 | not | | | | − |
| | saturated | | | | |
| | at 100 nM | | | | |

* = > 1 nM;
** = 0.05-1 nM;
*** 0.02-0.05 nM;
**** = <0.02 nM
# = >10 nM;
## = 1-10 nM;
### = <1 nM
n.d. = not determinable
− = no inhibition;
+ = inhibition;
++ = medium inhibition;
+++ = strong inhibition

In addition to a direct conversation of each scFv-format antibody to an IgG format, certain IgG1-format antibodies of the Comparative Examples were also generated to comprise combinations of heavy-chain and light-chain variable domains that were not previously combined. Briefly, the IgG expression system is a binary vector system, with one vector encoding the heavy chain and the other vector encoding the light chain. For transfection, the two vectors are mixed in a defined molar ratio and transfection is performed using standard methods. In order to allow chain swapping the different combinations of heavy chain- and light chain-encoding vectors were mixed and transfected using standard methods. Such combinations of heavy-chain and light-chain variable domains were shown to bind as strongly to His-tagged IGSF11 as a native combination of heavy-chain and light-chain variable domains (FIG. 11), and also to inhibit the interaction between IGSF11 (VSIG3) and VSIR (VISTA) (FIG. 12).

The combination of heavy-chain and light-chain variable domains (and corresponding CDRs) for these antibodies of the invention are respectively set forth in Tables C and B above, and Table 5.2 summarises the binding and inhibition characteristics of these chain-swapped antibodies of the Comparative Examples, as determined by the same assays as summarised in Table 5.1.

TABLE 5.2
Characterisation of chain-swapped antibodies of the
Comparative Examples to IGSF11 (VSIG3) inhibition
of binding between IGSF11 (VSIG3) and VSIR (VISTA)

| | Binding: | | |
|---|---|---|---|
| | HIS- | | |
| | IGSF11 | Affinity: | Inhibition: |
| | EC50 | SPR | IGSF11- |
| Antibody | (nM) | Kd (nM) | VSIR |
| B1 | **** | 250-500 | +++ |
| B2 | **** | 100-250 | +++ |
| B3 | **** | NT | +++ |
| B4 | **** | NT | +++ |
| B5 | **** | NT | +++ |
| B6 | **** | NT | +++ |
| B7 | **** | NT | +++ |
| B8 | **** | NT | +++ |

* = >1 nM:
** = 0.05-1 nM;
*** = 0.02-0.05 nM;
**** = <0.02 nM
− = no inhibition;
+ = inhibition;
++ = medium inhibition;
+++ = strong inhibition
NT = not tested

### COMPARATIVE EXAMPLE 6: DETECTION OF IGSF11 (VSIG3) USING AN ANTIBODY OF THE COMPARATIVE EXAMPLES

The antibodies of the Comparative Examples can detect IGSF11 expressed on the surface of cells.

Antibodies of the Comparative Examples in IgG1- or scFv-FC-format (eg, those from Table 3 recloned as described in Example 5) can also be used to specifically detect IGSF11 (VSIG3) expressed on the surface of tumour cells. FACS detection of lung H23 cells transiently transfected with negative control siRNA or IGSF11 knock-down siRNA is conducted, using a APC-labelled anti-human IgG as a secondary antibody. Cells knocked-down for IGSF11 (VSIG3) show reduced fluorescence compared to wild-type (ie, IGSF11-positive) cells. Alternatively, HEK-Freestyle or Expi293 cells (Invitrogen) are transfected with empty plasmid construct or plasmid constructs expressing the cDNA of IGSF11. Antibodies specific for IGSF11 show positive surface staining of the IGSF11-overexpressing HEK cells, compared to the control cells mock-transfected with empty plasmid.

Such antibodies of the Comparative Examples can be used to investigate the protein expression of IGSF11 (VSIG3) on the surface of one or more of the other tumour cell lines tested in Example 2 (eg, by FC/FACS or immunohistochemistry), and the amount of IGSF11 (VSIG3) detected by such antibodies can be associated with the degree of resistance each cell lines shows to cell-mediated immune response.

In particular, IgG1-format antibodies of the Comparative Examples were detected by FACS to bind to tumour cells known to express IGSF11 (eg lung cancer cell line DMS 273 and melanoma cell line M579-A2-luc), and binding was not detected if the cells were treated with IGSF11 siRNA (FIG. 13). No such binding (or IGSF11 siRNA-effect) was observed for the cancer cell line CL-11 which does not express IGSF11 (see FIG. 7).

Using such FACS binding assay, EC50s of binding of antibodies of the Comparative Examples to DMS 273 and to recombinant HEK cells expressing IGSF11 (“HEK-OE”) were determined (Table 6.1; “NA” EC50 not available from curve).

TABLE 6.1
EC50 of binding of antibodies of the Comparative
Examples to IGSF11-exressing cells

| | Binding: | Binding: |
|---|---|---|
| | DMS 273 | HEX-OE |
| | EC50 | EC50 |
| Antibody | (nM) | (nM) |
| A-015 | ND | ND |
| A-006 | >50 | 10-50 |
| A-007 | <10 | <10 |
| A-011 | ND | >50 |
| A-012 | 10-50 | 10-50 |
| A-024 | <10 | 10-50 |
| A-026 | ND | >50 |
| A-027 | ND | >50 |
| B-001 | ND | 10-50 |
| B-002 | >50 | 10-50 |
| A-013 | >50 | 10-50 |
| A-022 | 10-50 | 10-50 |
| A-035 | NA | >50 |

ND = not determinable

Such results demonstrate the ability of antibodies of the Comparative Examples to detected the expression of IGSF11 (VSIG3) protein and their utility to determine increased resistance of a cell against an immune response, such as of a cancer cell.

IGSF11 can be expressed by immune cells (eg monocytes from the PBMC of a healthy donor; FIG. 6). Monocytes from the PBMC of two healthy donors (A and B) were stained with the described concentration of the anti-IGSF11 scFv format of antibody A-015 (see Example 5) or mouse IgG2a isotype control antibody for 30 min at 4° C., and then detected by FACS using a fluorescently-labeled secondary antibody (gated on CD14 monocytic marker). Analogous methodology can be used to investigate (eg to detect) IGSF11 expression on macrophages (especially TAMs, but also MDSCs, immature DCs etc) present in samples (eg tumour samples) from cancer patients.

### EXAMPLE 7: IMMUNO-MODULATORY FUNCTION OF ANTIBODIES THAT BIND TO HUMAN IGSF11 (VSIG3)

Antibodies that bind IGSF11, in particular that bind the IgC2 domain (or IgV domain) of IGSF11 in human IgG1-format (eg, those from Comparative Example 5) are found to sensitise human tumour cells towards TIL-mediated cytotoxicity. Analogously to the methodology described in Comparative Example 1 for siRNA knockdown, human tumour cells from one or more of the following cell lines: MCF7, SW480, M579-A2, KMM1, PANC-1, CaCo2, MDA-MB-231, HCT-116, H23, A54 (each, containing a luc reporter) are treated with 0.1, 1, 5, 10, 30 or 60 ug of IgG1- or scFv-FC-format antibodies (eg from those described in Comparative Example 5) and 72h thereafter are co-cultured with cytotoxic T cells (eg TILs). Compared to samples exposed to non-specific control IgG antibodies, tumour cell lines that are exposed to the IgG1-format antibodies that bind IGSF11, in particular that bind the IgC2 domain (or IgV domain) of IGSF11 display increased cytotoxicity (eg, decreased viability).

In addition, addition of antibodies that bind IGSF11, in particular that bind the IgC2 domain (or IgV domain) of IGSF11 to a co-culture of CD3+ T cells and luciferase-expressing MDA-MB-231 breast cancer cells that were transfected to express IGSF11 (and further including an anti-EPCAM-Anti CD3 bi-specific antigen-binding construct consisting of two scFvs, “BiTE”), was shown to induce increased lysis of the cancer cells; where such antibodies are about as active as addition of an anti-PD-Li checkpoint inhibitor antibody, and almost as active as an anti-VISTA antibody, the T cell receptor of IGSF11 (FIG. 14).

For this assay, 6,000 IGSF11-expressing MDA-MB-231-luc cells were seeded into every well of a flat bottom 96-well plate and incubated for 24 h. Then 1×10⁵ naive CD3+ T cells (freshly isolated from PBMCs) were added and co-cultured with the tumour cells in the presence of 2-8 ng/mL EpCAM×CD3 BiTE (solitomab, AmGen) and 40 ug/mL of the test monoclonal antibody. Antibodies against PD-Li and VISTA (atezolizumab, Roche; VSTB112, Janssen; respectively) were included as a positive control; VISTA was included as a positive control for the IGSF11/VISTA axis, as VISTA is the receptor for IGSF11 on the T cell side). As a negative control a human IgG1 isotype control antibody was used. The tested anti-IGSF11 that bind IGSF11, in particular that bind the IgC2 domain, eg A-006 and A-012, are shown to block the IGSF11/VISTA interaction in ELISA (see Comparative Example 5). After 3 days of co-culture, the amount of living MDA-MB-231-luc IGSF11-expressing tumour cells was quantified via luciferase read-out. For this, the supernatants were removed and the plate was washed once with PBS. The cells were lysed by addition of 40 uL cell lysis buffer for 15 min at room temperature. Then 45 uL of luc buffer containing the luciferase substrate D-luciferin were added to the lysed tumour cells and the bioluminescent signal was detected via a Tecan Spark 20M plate reader at an integration time of 100 ms.

IGSF11-expressing MDA-MB-231-luc cells were generated by transfection of an IGSF11-encoding lentiviral vector based on p443MYCIN (proQinase). Expression of IGSF11, EpCAM and PD-L1 by the transfected MDA-MB-231-luc cells was confirmed by FACS staining with the applicable primary and secondary antibodies (FIG. 15).

Such results further demonstrate the ability of antibodies that bind IGSF11, in particular that bind the IgC2 domain (or IgV domain) of IGSF11 to sensitise a cell to an immune response, such as to sensitise a cancer cell to a cell-mediated immune response.

### EXAMPLE 8 (PROPHETIC): ANTIBODIES THAT BIND TO HUMAN IGSF11 (VSIG3) ATTENUATE THE INHIBITION OF CYTOKINE AND CHEMOKINE PRODUCTION BY HUMAN PBMCS

IGSF11, particular IgC2 domain (or IgV domain) of IGSF11, (Fc protein) can inhibit the production of IL-2 by stimulated T cells. 96-well culture plates were coated with anti-CD3 antibody (clone OKT3; 2.5 ug/mL; eBioscience, Cat #16-0037-81) along with 10 ug/mL of the respective recombinant Fc-proteins: either IGSF11-Fc (R&D systems; #9229-VS-050) or PD-L1-Fc (R&D systems; #156-B7) or IgG1-Fc control (R&D systems, #110-HG-100). Approximately 50,000 T cells, purified from PBMC of a healthy donor, were added to each well to test the inhibitory or stimulatory effect of Fc proteins on T cell activation. After 2 days of incubation at 37° C., plates were centrifuged and the supernatant was collected to measure IL-2 production (eg, human IL-2 Quantikine ELISA Kit, R&D Systems, Cat #: D2050). Compared to unstimulated cells (no addition of anti-CD3 antibody or Fc proteins) showed basal level of IL-2 production, which was markedly increased in cells that were stimulated with anti-CD3 antibody alone. In comparison, addition of IGSF11-Fc protein significantly (P<0.05) decreased the IL-2 production from activated T cells, even more than the decrease in IL-2 production upon addition of PD-L1-Fc (P<0.01) (FIG. 5).

Wang et al (2017) described the inhibition of cytokine and chemokine production by PBMCs, in particular of CCL5/RANTES, MIP-1 alpha/beta, IL-17A and CXCL11/I-TAC. Therefore, using such an assay, it can be further shown that pre-treatment of recombinant human soluble IGSF11 (VSIG3) (ECD-IgG1-Fc fusion, in particular Fc fusions of the IgC2 domain (or IgV domain) of IGSF11) with IgG1-format antibodies that bind IGSF11, in particular that bind the IgC2 domain (or IgV domain) of IGSF11, attenuates the inhibition of cytokine and chemokine production by anti-CD3 activated human PBMCs when exposed to such pre-treated IGSF11 (VSIG3); in particular, such pre-treatment increases the relative production of CCL5/RANTES, MIP-1alpha/beta, IL-17A and/or CXCL11/I-TAC of human PBMCs, as measured using the Proteome Profiler™ Human XL Cytokine Array Kit (R&D Systems, Catalog #ARY022B) and/or Quantikine® ELISA Kits (R&D Systems, Catalog #D1700, DRN00B, DCX110, and DMA00).

Such results further demonstrate the ability of antibodies that bind IGSF11, in particular that bind the IgC2 domain (or IgV domain) of IGSF11, to sensitise a cell to an immune response, such as to sensitise a cancer cell to a cell-mediated immune response.

### EXAMPLE 9 (PROPHETIC): ANTIBODIES THAT BIND TO HUMAN IGSF11 (VSIG3) ATTENUATE THE INHIBITION OF HUMAN T CELL ACTIVATION BY IGSF11 (VSIG3)

Wang et al (2017) described that IGSF11 (VSIG3) inhibited anti-CD3 induced IL-2, IFN-g, and IL-17 production by human CD3+ T cells in a dose-dependent manner. Analogous to the experiment described by Wang et al, by pre-treatment of the IGSF11 (VSIG3) with IgG1-format antibodies that bind IGSF11, in particular that bind the IgC2 domain (or IgV domain) of IGSF11, it can be shown that such antibodies attenuate the inhibition (eg, activate) the production of one or more cytokines by human CD+ T cells, for example pro-inflammatory cytokines such as IL-2, IFN-gamma, and IL-17, as well as attenuate the inhibition of (eg, activate) human CD3+ T cell proliferation.

Such results further demonstrate the ability of antibodies that bind IGSF11, in particular that bind the IgC2 domain (or IgV domain) of IGSF11, to sensitise a cell to an immune response, such as to sensitise a cancer cell to a cell-mediated immune response.

### EXAMPLE 10 (PROPHETIC): TUMOUR RESISTANCE TO ADOPTIVE T CELL TRANSFER CAN BE OVERCOME BY IGSF11 (VSIG3) INHIBITION IN VIVO

Inhibition of IGSF11 (VSIG3) can be used to overcome resistance of tumour cells to anti-tumour immune response(s) in an in vivo model. IGSF11 (VSIG3) is stably knocked down in a primary cancer cell line (eg, H23, A549 or M579-A2) using IGSF11-specific shRNA (shIGSF11) or the control non-targeting shRNA sequence (shCtrl). The transduced cell lines are produced briefly as follows: lentiviral transduction particles expressing an shRNA targeting IGSF11 mRNA or control shRNA (Sigma-Aldrich, eg The RNAi Consortium (TRC) numbers: TRCN0000431895, TRCN0000428521 or TRCN0000425839 for IGSF11 CDS, and SHC002 for control) are used for transduction. 5×10e4 eg H23-Luc cells are seeded in a 6 well plate in DMEM 10% FCS 1% P/S. After 24h, lentiviral particles are added using multiplicity of infection (MOI)=2. 48h from transduction, cells are put under positive selection using 0.4 μg/ml puromycin. First, the shIGSF11 or shCtrl transduced tumour cells are co-cultured with HLA-matched TILs, and the extent of T cell-mediated killing monitored using in vitro real-time live-cell microscopy. TILs that show increased killing efficacy towards shIGSF11 depleted tutor cells compared to shCtrl are identified. Second, the shIGSF11 and shCtrl transduced tumour cells are subcutaneously injected into the left and the right flank, respectively, of NSG immune deficient mice, and adoptive cell transfer of the identified TIL or PBS injection is applied i.v. once per week. TIL-treatment causes retardation of tumour growth in IGSF11-impaired tumour cells compared to shCtrl-transduced cells. Consistent with the in vitro data, no difference in the tumour growth kinetic between shCtrl and shIGSF11 is observed in PBS-treated mice.

### EXAMPLE 11 (PROPHETIC): TUMOUR RESISTANCE TO ADOPTIVE T CELL TRANSFER CAN BE OVERCOME, IN VIVO, BY TREATMENT WITH AN ANTIBODY THAT BINDS IGSF11

Analogous to Example 10, H23 cells are subcutaneously injected into a flank of NSG immune deficient mice, and adoptive cell transfer of the identified TIL or PBS injection is applied i.v. once per week. Mice then received 50 ug/dose or 200 ug/dose of an antibody of the invention, or vehicle control, twice per week for 4 weeks. Antibody treatment causes retardation of tumour growth in this adoptive T cell transfer model compared to administration of vehicle alone.

Such results further demonstrate the ability of antibodies that bind IGSF11, in particular that bind the IgC2 domain (or IgV domain) of IGSF11, to sensitise a cell to an immune response, such as to sensitise a cancer cell to a cell-mediated immune response.

### EXAMPLE 12: RELEVANCE OF IGSF11 TO TUMOUR GROWTH IN VIVO, AND INHIBITION OF TUMOUR GROWTH IN VIVO BY TREATMENT WITH AN ANTIBODY THAT BINDS IGSF11

To further validate IGSF11 as an immune-checkpoint molecule, an experiment was conducted to monitor tumour growth in a syngeneic mouse model using MC38 murine tumour cells (which naturally expresses murine IGSF11). MC38 tumour cell lines were generated that carry either an IGSF11 CRISPR-knockout (“KO”), or are transduced with murine IGSF11 to stably overexpress murine IGSF11 (“OE”) or with the empty vector (mock transduction) to generate a wild-type murine IGSF11 cell line (“WNT”), as briefly described in the following:

Knockout of IGSF11 in MC38 cells was done by CRISPR-Cas9. Guide RNAs (gRNA) were designed to target the exon-intron junction of a common target exon to generate insertion-deletions (Indels) resulting in frameshifts or exon skipping. MC38 cells were nucleofected with plasmids coding for Cas9 and gRNA. Single clones were isolated by limiting dilution and Indels were verified by next-generation sequencing.

IGSF11 overexpressing and wild-type cell lines were generated by by ProQinase GmbH (Germany). MC38 cells (Kerafast, Cat #ENH204-FP) were transduced with lentivirus encoding for murine IGSF11 (Uniprot #POC673) as well as with the empty vector (mock transduction) using the standard ProQinase transduction procedure. Briefly, HEK293T packaging cells were transfected with p443MYCIN-IGSF11 mM or empty vector p443MYCIN (containing neomycin resistance). 48 h post-transfection sterile-filtered HEK293T-supernatants containing the respective lentivirus were added to parental MC38 cells. After 3 days, transduced MC38 cells were split and subjected to 3 mg/mL Geneticin to select for transduced cells. After 3 split cycles, cells were tested for IGSF11 expression by flow cytometry.

These three types of tumour cells were each injected into immune-competent mice as follows: Ten animals (female C57BL/6N mice, Charles River Laboratories, Germany) per tumour cell line were anesthetized and received 1×10\hat{ }6 tumour cells of the respective cell line (100 ul of a suspension in PBS with 50% Matrigel) by injection into the left flank. The absolute tumour volumes were determined on the day of injection and twice weekly. All animals were sacrificed 15 days after tumour cell implantation. Tumour volume was determined and five tumours from each treatment group were sampled for flow cytometry analysis.

As an immune checkpoint molecule (associated with resistance of tumour cells to an immune response), the tumour growth curves of the WT, KO and OE cells separate, wherein KO tumours are rejected better by the immune systems and IGSF11 overexpressing cells (OE) show a stronger growth as they suppress the immune system of the mouse. Indeed, the KO cell line shows about 40% tumour growth inhibition compared to the WT mock-transduction control (FIG. 16A). Analysis of the tumours showed a significant decrease of intra-tumoural (CD11b+/Ly6G+) granulocytic subsets of myeloid-derived suppressor cells (gMDSCs) in tumours of mice injected with KO tumour cells compared to those injected with either OE tumour cells or WT (mock control) tumour cells (FIG. 16B), as well as an increase of intra-tumoural (CD8+) cytotoxic T cells in tumours of mice injected with KO tumour cells compared to those injected with OE tumour cells. (FIG. 16C). Further investigation into mechanistic understanding (by e.g. tumour cytokine analysis, T cell depletion, combination with PD-1) is conducted. This model is then shown to be responsive to treatment with an anti-VISTA antibody (eg, VSTB112, Janssen), as well as to treatment with antibodies that bind IGSF11, in particular that bind the IgC2 domain (or IgV domain) of IGSF11, (cross reactive to murine IGSF11, see Table 2 and Table 13.2).

### EXAMPLE 13: GENERATION OF FURTHER ANTIBODIES THAT BIND TO HUMAN IGSF11 (VSIG3)

The inventors identified human Fab antibodies that bind human IGSF11 (VSIG3) by selection, using phage display, from a fully-human antibody gene library using recombinant protein and IGSF11 expressing cell lines.

The fully-human antibody gene library displays fully human antibodies in Fab format on M13 phage by having the antibody Fd region genetically fused to the N-terminus of the phage gene3. Gene3 and the used master genes are encoded on a phagemid. The antibody sequences encompass the variable regions of several selected human heavy chain, kappa and lambda germline genes, wherein CDR-H3s and -L3s are diversified according to the amino acid compositions of rearranged human antibody repertoire. Due to the mainly synthetic nature of the library the occurrence of known sequence hot spots is reduced. The total library size is ca. 10\hat{ }10 different antibodies.

Briefly, the phage library was blocked with 2×ChemiBLOCKER (Merck Millipore), biotinylated recombinant human IGSF11 (EC domain) was added at a concentration of 40-50 nM and incubated for 1 h at room temperature. Antibody-expressing phage bound to recombinant ECD-IGSF11 were separated using streptavidin magnetic beads (Dynabeads M-280, ThermoFisher) and washed with PBST. Anti-IGFS11 antibody-expressing phage were eluted from the beads using 10 ug/mL Trypsin and used to infect mid-logarithmic E. coli TG1 for phage amplification.

Two or three rounds of enrichment using biotinylated recombinant ECD-IGSF11 were performed, whereas the first round of enrichment was performed with human ECD-IGSF11, the second round on murine ECD-IGSF11 and the third round on human ECD-IGFS11.

In order to identify IGSF11-specific binding ABPs, monoclonal Fabs expressed from E. coli were generated from the enriched set of anti-IGFS11 antibody-expressing phage. After bacterial lysis, the Fabs were tested for their binding properties and cross-reactivity profile on recombinant ECD-IGSF11 (human, mouse and cynomolgus monkey) by standard ELISA. Briefly, biotinylated recombinant ECD-IGSF11 from human, mouse, or cynomolgus monkey were immobilized at 2 ug/mL on a streptavidin-coated 384-well Maxisorp plate. The surface was blocked with 2% (w/v) skim milk powder in PBST. After three wash cycles with PBST, a bacterial lysate of each of the anti-IGSF11 Fabs in 2% (w/v) skim milk powder was applied to the immobilized IGSF11 and incubated for 1 h. After removing all unbound Fab by 3 wash cycles with PBST, bound Fab antibodies were detected with a goat anti-human Fab antibody conjugated with horseradish peroxidase. After three wash cycles with PBST the ELISA was developed with TMB substrate.

ABPs of this example were converted to IgG briefly as follows: individual heavy and light chain variable-region sequences from the Fab-format ABPs were genetically fused to wildtype human IgG1 and kappa or lambda constant-regions, respectively. Expi293 cells (ThermoFisher) were transiently transfected with DNA sequences encoding the applicable combination of heavy and light chains of IgG1, according to the manufacturer's instructions, and cultured under conditions suitable to express IgG1-format ABP (antibody). Cell supernatants were harvested 5 days post transfection and the expressed antibody purified via Protein A affinity chromatography (GE Healthcare). Purified antibodies (IgG1-format ABPs of this example) were re-buffered into PBS pH7.4.

Cell binding of ABP in IgG1 format on recombinantly overexpressing and endogenously expressing tumour cell lines was tested by standard flow cytometry (FC). Briefly, the cells were stained with a dilution series of the IgG1-format antibodies. Unbound antibodies were removed by washing the cells three time with FACS buffer. Bound antibodies were detected with a mouse anti-human IgG antibody conjugated with AlexaFluor647.

Fab antibodies of this Example that selectively bind the ECD of human, murine and cynomolgus monkey IGSF11 protein over streptavidin are identified and described in Tables 13, showing for each such antibody the heavy chain and light chain CDR sequences and variable region sequences comprised in each such antibody as well as nucleic acid sequences encoding for such variable regions (Table 13.1A), and the identification of the human germ-line genes for the variable regions (Table 13.1B). The degree of binding of each such antibody to human, murine and cynomolgus monkey IGSF11 protein (and to irrelevant antigen), as determined by the ELISA, and to mouse IGSF11 protein expressed by cells, as determined by flow cytometry (FC), is shown in Table 13.2.

TABLE 13.1A
Amino acid sequences of CDR and variable regions of ABPs of this Example, as well as
nucleic acid sequences encoding variable regions of ABPs of this Example.

| | | Sequence | SEQ |
|---|---|---|---|
| Antibody | Region | 1.......10.......20.......30.......40.......50 | ID NO. |
| C-001 | H-CDR1 | SYAMS | 391 |
| | H-CDR2 | AISGSGGSTYYADSVKG | 392 |
| | H-CDR3 | IHRPLDV | 393 |
| | VH (aa) | EVQLLESGGGLVQPGGSLRLSCAASGFTFSSYAMSWVRQAPGKGLEWVSA | 394 |
| | | ISGSGGSTYYADSVKGRFTISRDNSKNTLYLQMNSLRAEDTAVYYCARIH | |
| | | RPLDVWGQGTLVTVSS | |
| | L-CDR1 | SGSSSNIGNNYVS | 395 |
| | L-CDR2 | DNNKRPS | 396 |
| | L-CDR3 | GSWLEERSQYV | 397 |
| | VL (aa) | ASVLTQPPSVSAAPGQKVTISCSGSSSNIGNNYVSWYQQLPGTAPKLLIY | 398 |
| | | DNNKRPSGIPDRFSGSKSGTSATLGITGLQAEDEADYYCGSWLEERSQYV | |
| | | FGGGTKLTVL | |
| | VH (DNA) | GAAGTTCAGCTGCTGGAAAGCGGTGGTGGTCTGGTTCAGCCTGGTGGTAG | 399 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTCTATTATTGTGCGCGCATCCAT | |
| | | CGTCCACTGGATGTTTGGGGCCAGGGCACCCTGGTTACTGTCTCGAGC | |
| | VL (DNA) | GCGAGCGTTCTGACCCAGCCTCCGAGCGTTAGCGCAGCACCGGGTCAGAA | 400 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCGGTTCTTGGCTGGAAGAACGTTCTCAGTACGTG | |
| | | TTCGGCGGTGGTACCAAGTTAACCGTGCTG | |
| C-002 | H-CDR1 | SYAMS | 401 |
| | H-CDR2 | AISGSGGSTYYADSVKG | 402 |
| | H-CDR3 | DLSSGWGHAFDI | 403 |
| | VH (aa) | EVQLLESGGGLVQPGGSLRLSCAASGFTFSSYAMSWVRQAPGKGLEWVSA | 404 |
| | | ISGSGGSTYYADSVKGRFTISRDNSKNTLYLQMNSLRAEDTAVYYCARDL | |
| | | SSGWGHAFDIWGQGTMVPVSS | |
| | L-CDR1 | SGSSSNIGNNYVS | 405 |
| | L-CDR2 | DNNKRPS | 406 |
| | L-CDR3 | LSYTTSEHHLV | 407 |
| | VL (aa) | ASVLTQPPSVSAAPGQKVTISCSGSSSNIGNNYVSWYQQLPGTAPKLLIY | 408 |
| | | DNNKRPSGIPDRFSGSKSGTSATLGITGLQAEDEADYYCLSYTTSEHHLV | |
| | | FGGGTKLTVL | |
| | VH (DNA) | GAAGTTCAGCTGCTGGAAAGCGGTGGTGGTCTGGTTCAGCCTGGTGGTAG | 409 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTGTATTACTGTGCCAGAGACTTA | |
| | | AGTAGTGGTTGGGGTCATGCTTTTGATATCTGGGGCCAGGGGACAATGGT | |
| | | CCCCGTCTCGAGC | |
| | VL (DNA) | GCGAGCGTTCTGACCCAGCCTCCGAGCGTTAGCGCAGCACCGGGTCAGAA | 410 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGTCTTACACTACTTCTGAACATCATCTGGTG | |
| | | TTCGGCGGTGGTACCAAGTTAACCGTGCTG | |
| C-003 | H-CDR1 | SYAMS | 411 |
| | H-CDR2 | AISGSGGSTYYADSVKG | 412 |
| | H-CDR3 | DSRDAYGVAFDL | 413 |
| | VH (aa) | EVQLLESGGGLVQPGGSLRLSCAASGFTFSSYAMSWVRQAPGKGLEWVSA | 414 |
| | | ISGSGGSTYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 415 |
| | L-CDR2 | DNNKRPS | 416 |
| | L-CDR3 | LSYTSSQYV | 417 |
| | VL (aa) | ASVLTQPPSVSAAPGQKVTISCSGSSSNIGNNYVSWYQQLPGTAPKLLIY | 418 |
| | | DNNKRPSGIPDRFSGSKSGTSATLGITGLQAEDEADYYCLSYTSSQYVFG | |
| | | GGTKLTVL | |
| | VH (DNA) | GAAGTTCAGCTGCTGGAAAGCGGTGGTGGTCTGGTTCAGCCTGGTGGTAG | 419 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTATATTACTGTGCGAGAGACTCA | |
| | | AGAGATGCCTACGGGGTTGCTTTTGATCTCTGGGGCCAAGGGACAATGGT | |
| | | CACCGTCTCGAGC | |
| | VL (DNA) | GCGAGCGTTCTGACCCAGCCTCCGAGCGTTAGCGCAGCACCGGGTCAGAA | 420 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGTCTTACACTTCTTCTCAGTACGTGTTCGGC | |
| | | GGTGGTACCAAGTTAACCGTGCTG | |
| C-004 | H-CDR1 | SYAMS | 421 |
| | H-CDR2 | AISGSGGSTYYADSVKG | 422 |
| | H-CDR3 | DSRDAYGVAFDL | 423 |
| | VH (aa) | EVQLLESGGGLVQPGGSLRLSCAASGFTFSSYAMSWVRQAPGKGLEWVSA | 424 |
| | | ISGSGGSTYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 425 |
| | L-CDR2 | DNNKRPS | 426 |
| | L-CDR3 | LTWTGAGRIFV | 427 |
| | VL (aa) | ASVLTQPPSVSAAPGQKVTISCSGSSSNIGNNYVSWYQQLPGTAPKLLIY | 428 |
| | | DNNKRPSGIPDRFSGSKSGTSATLGITGLQAEDEADYYCLTWTGAGRIFV | |
| | | FGGGTKLTVL | |
| | VH (DNA) | GAAGTTCAGCTGCTGGAAAGCGGTGGTGGTCTGGTTCAGCCTGGTGGTAG | 429 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTATATTACTGTGCGAGAGACTCA | |
| | | AGAGATGCCTACGGGGTTGCTTTTGATCTCTGGGGCCAAGGGACAATGGT | |
| | | CACCGTCTCGAGC | |
| | VL (DNA) | GCGAGCGTTCTGACCCAGCCTCCGAGCGTTAGCGCAGCACCGGGTCAGAA | 430 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGACTTGGACTGGTGCAGGTCGTATCTTTGTG | |
| | | TTCGGCGGTGGTACCAAGTTAACCGTGCTG | |
| C-005 | H-CDR1 | NAWMS | 431 |
| | H-CDR2 | RIKSKTDGGTTDYAAPVKG | 432 |
| | H-CDR3 | LGIYSGFDY | 433 |
| | VH (aa) | EVQLVESGGGLVKPGGSLRLSCAASGFTFSNAWMSWVRQAPGKGLEWVGR | 434 |
| | | IKSKTDGGTTDYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 435 |
| | L-CDR2 | QDSKRPS | 436 |
| | L-CDR3 | HSYTGKPSQW | 437 |
| | VL (aa) | SYELTQPPSVSVSPGQTASITCSGDKLGDKYASWYQQKPGQSPVLVIYQD | 438 |
| | | SKRPSGIPERFSGSNSGNTATLTISGTQAEDEADYYCHSYTGKPSQVVFG | |
| | | GGTKLTVL | |
| | VH (DNA) | GAAGTTCAGCTGGTTGAAAGCGGTGGTGGTCTGGTTAAACCTGGTGGTAG | 439 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTATCTACTCTGGTTTTGATTACTGGGGCCAGGGCACCCTGGTTAC | |
| | | TGTCTCGAGC | |
| | VL (DNA) | AGCTATGAACTGACCCAGCCTCCGAGCGTTAGCGTTAGTCCGGGTCAGAC | 440 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| C-006 | H-CDR1 | SYAMS | 441 |
| | H-CDR2 | AISGSGGSTYYADSVKG | 442 |
| | H-CDR3 | HWVSYGPFDY | 443 |
| | VH (aa) | EVQLLESGGGLVQPGGSLRLSCAASGFTFSSYAMSWVRQAPGKGLEWVSA | 444 |
| | | ISGSGGSTYYADSVKGRFTISRDNSKNTLYLQMNSLRAEDTAVYYCARHW | |
| | | VSYGPFDYWGQGTLVTVSS | |
| | L-CDR1 | RASQSISSYLN | 445 |
| | L-CDR2 | AASSLQS | 446 |
| | L-CDR3 | QQSHQSPPIT | 447 |
| | VL (aa) | DIQMTQSPSSLSASVGDRVTITCRASQSISSYLNWYQQKPGKAPKLLIYA | 448 |
| | | ASSLQSGVPSRFSGSGSGTDFTLTISSLQPEDFATYYCQQSHQSPPITFG | |
| | | QGTKVEIK | |
| | VH (DNA) | GAAGTTCAGCTGCTGGAAAGCGGTGGTGGTCTGGTTCAGCCTGGTGGTAG | 449 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTATATTACTGTGCGAGACATTGG | |
| | | GTCAGCTATGGCCCTTTTGACTACTGGGGCCAGGGCACCCTGGTCACCGT | |
| | | CTCGAGC | |
| | VL (DNA) | GATATTCAGATGACCCAGAGTCCGAGCAGCCTGAGCGCAAGCGTTGGTGA | 450 |
| | | TCGTGTTACCATTACCTGTCGTGCAAGCCAGAGCATTAGCAGCTATCTGA | |
| | | ATTGGTATCAGCAGAAACCGGGTAAAGCACCGAAACTGCTGATTTATGCA | |
| | | GCAAGCAGCCTGCAGAGCGGTGTTCCGAGCCGTTTTAGCGGATCCGGTAG | |
| | | CGGCACCGATTTTACCCTGACCATTAGCAGTCTGCAGCCGGAAGACTTTG | |
| | | CCACCTATTATTGCCAGCAGTCTCATCAGTCTCCGCCGATCACTTTCGGC | |
| | | CAGGGTACCAAAGTGGAAATTAAG | |
| C-007 | H-CDR1 | SYAMS | 451 |
| | H-CDR2 | AISGSGGSTYYADSVKG | 452 |
| | H-CDR3 | IYRAFDY | 453 |
| | VH (aa) | EVQLLESGGGLVQPGGSLRLSCAASGFTFSSYAMSWVRQAPGKGLEWVSA | 454 |
| | | ISGSGGSTYYADSVKGRFTISRDNSKNTLYLQMNSLRAEDTAVYYCARIY | |
| | | RAFDYWGQGTLVTVSS | |
| | L-CDR1 | SGSSSNIGNNYVS | 455 |
| | L-CDR2 | DNNKRPS | 456 |
| | L-CDR3 | QLYEEEHSTWV | 457 |
| | VL (aa) | ASVLTQPPSVSAAPGQKVTISCSGSSSNIGNNYVSWYQQLPGTAPKLLIY | 458 |
| | | DNNKRPSGIPDRFSGSKSGTSATLGITGLQAEDEADYYCQLYEEEHSTWV | |
| | | FGGGTKLTVL | |
| | VH (DNA) | GAAGTTCAGCTGCTGGAAAGCGGTGGTGGTCTGGTTCAGCCTGGTGGTAG | 459 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTCTATTATTGTGCGCGCATCTAC | |
| | | CGTGCATTTGATTACTGGGGCCAGGGCACCCTGGTTACTGTCTCGAGC | |
| | VL (DNA) | GCGAGCGTTCTGACCCAGCCTCCGAGCGTTAGCGCAGCACCGGGTCAGAA | 460 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCAGCTGTACGAAGAAGAACATTCTACTTGGGTG | |
| | | TTCGGCGGTGGTACCAAGTTAACCGTGCTG | |
| C-008 | H-CDR1 | SYAMS | 461 |
| | H-CDR2 | AISGSGGSTYYADSVKG | 462 |
| | H-CDR3 | DNGHNRD | 463 |
| | VH (aa) | EVQLLESGGGLVQPGGSLRLSCAASGFTFSSYAMSWVRQAPGKGLEWVSA | 464 |
| | | ISGSGGSTYYADSVKGRFTISRDNSKNTLYLQMNSLRAEDTAVYYCTTDN | |
| | | GHNRDWGQGTLVTVSS | |
| | L-CDR1 | SGSSSNIGNNYVS | 465 |
| | L-CDR2 | DNNKRPS | 466 |
| | L-CDR3 | GTWTRSSGV | 467 |
| | VL (aa) | ASVLTQPPSVSAAPGQKVTISCSGSSSNIGNNYVSWYQQLPGTAPKLLIY | 468 |
| | | DNNKRPSGIPDRFSGSKSGTSATLGITGLQAEDEADYYCGTWTRSSGVFG | |
| | | GGTKLTVL | |
| | VH (DNA) | GAAGTTCAGCTGCTGGAAAGCGGTGGTGGTCTGGTTCAGCCTGGTGGTAG | 469 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTGTATTACTGTACCACAGATAAT | |
| | | GGCCACAATAGGGACTGGGGCCAGGGCACCCTGGTCACCGTCTCGAGC | |
| | VL (DNA) | GCGAGCGTTCTGACCCAGCCTCCGAGCGTTAGCGCAGCACCGGGTCAGAA | 470 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCGGTACTTGGACTCGTTCTTCTGGTGTGTTCGGC | |
| | | GGTGGTACCAAGTTAACCGTGCTG | |
| C-009 | H-CDR1 | SYAMS | 471 |
| | H-CDR2 | AISGSGGSTYYADSVKG | 472 |
| | H-CDR3 | GHHHGVYYFYAMDL | 473 |
| | VH (aa) | EVQLLESGGGLVQPGGSLRLSCAASGFTFSSYAMSWVRQAPGKGLEWVSA | 474 |
| | | ISGSGGSTYYADSVKGRFTISRDNSKNTLYLQMNSLRAEDTAVYYCARGH | |
| | | HHGVYYFYAMDLWGQGTLVTVSS | |
| | L-CDR1 | SGDKLGDKYAS | 475 |
| | L-CDR2 | QDSKRPS | 476 |
| | L-CDR3 | QSYSGSSTLHV | 477 |
| | VL (aa) | SYELTQPPSVSVSPGQTASITCSGDKLGDKYASWYQQKPGQSPVLVIYQD | 478 |
| | | SKRPSGIPERFSGSNSGNTATLTISGTQAEDEADYYCQSYSGSSTLHVFG | |
| | | GGTKLTVL | |
| | VH (DNA) | GAAGTTCAGCTGCTGGAAAGCGGTGGTGGTCTGGTTCAGCCTGGTGGTAG | 479 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTCTATTATTGTGCGCGCGGTCAT | |
| | | CATCATGGTGTTTACTACTTTTACGCAATGGATCTGTGGGGCCAGGGCAC | |
| | | CCTGGTTACTGTCTCGAGC | |
| | VL (DNA) | AGCTATGAACTGACCCAGCCTCCGAGCGTTAGCGTTAGTCCGGGTCAGAC | 480 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCAGTCTTACTCTGGTTCTTCTACTCTGCATGTGTTCGGC | |
| | | GGTGGTACCAAGTTAACCGTGCTG | |
| C-010 | H-CDR1 | SYAIS | 481 |
| | H-CDR2 | GIIPIFGTANYAQKFQG | 482 |
| | H-CDR3 | GYGEYYPAFDV | 483 |
| | VH (aa) | QVQLVQSGAEVKKPGSSVKVSCKASGGTFSSYAISWVRQAPGQGLEWMGG | 484 |
| | | IIPIFGTANYAQKFQGRVTITADESTSTAYMELSSLRSEDTAVYYCARGY | |
| | | GEYYPAFDVWGQGTLVTVSS | |
| | L-CDR1 | SGDKLGDKYAS | 485 |
| | L-CDR2 | QDSKRPS | 486 |
| | L-CDR3 | ASYAHTHSTWV | 487 |
| | VL (aa) | SYELTQPPSVSVSPGQTASITCSGDKLGDKYASWYQQKPGQSPVLVIYQD | 488 |
| | | SKRPSGIPERFSGSNSGNTATLTISGTQAEDEADYYCASYAHTHSTWVFG | |
| | | GGTKLTVL | |
| | VH (DNA) | CAGGTTCAGCTGGTTCAGAGCGGTGCAGAAGTTAAAAAACCGGGTAGCAG | 489 |
| | | CGTTAAAGTTAGCTGTAAAGCAAGCGGTGGCACCTTTAGCAGCTATGCAA | |
| | | TTAGCTGGGTTCGTCAGGCACCTGGTCAAGGTCTGGAATGGATGGGTGGT | |
| | | ATTATTCCGATTTTTGGCACCGCAAATTATGCCCAGAAATTTCAGGGTCG | |
| | | TGTTACCATTACCGCAGATGAAAGCACCAGCACCGCATATATGGAACTGA | |
| | | GCAGCCTGCGTAGCGAAGATACGGCCGTCTATTATTGTGCGCGCGGTTAC | |
| | | GGTGAATACTACCCAGCATTTGATGTTTGGGGCCAGGGCACCCTGGTTAC | |
| | | TGTCTCGAGC | |
| | VL (DNA) | AGCTATGAACTGACCCAGCCTCCGAGCGTTAGCGTTAGTCCGGGTCAGAC | 490 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCGCATCTTACGCACATACTCATTCTACTTGGGTGTTCGGC | |
| | | GGTGGTACCAAGTTAACCGTGCTG | |
| C-011 | H-CDR1 | SYAIS | 491 |
| | H-CDR2 | GIIPIFGTANYAQKFQG | 492 |
| | H-CDR3 | HSTPSFLQY | 493 |
| | VH (aa) | QVQLVQSGAEVKKPGSSVKVSCKASGGTFSSYAISWVRQAPGQGLEWMGG | 494 |
| | | IIPIFGTANYAQKFQGRVTITADESTSTAYMELSSLRSEDTAVYYCARHS | |
| | | TPSFLQYWGQGTLVTVSS | |
| | L-CDR1 | SGDKLGDKYAS | 495 |
| | L-CDR2 | QDSKRPS | 496 |
| | L-CDR3 | AVYPAHASARWV | 497 |
| | VL (aa) | SYELTQPPSVSVSPGQTASITCSGDKLGDKYASWYQQKPGQSPVLVIYQD | 498 |
| | | SKRPSGIPERFSGSNSGNTATLTISGTQAEDEADYYCAVYPAHASARWVF | |
| | | GGGTKLTVL | |
| | VH (DNA) | CAGGTTCAGCTGGTTCAGAGCGGTGCAGAAGTTAAAAAACCGGGTAGCAG | 499 |
| | | CGTTAAAGTTAGCTGTAAAGCAAGCGGTGGCACCTTTAGCAGCTATGCAA | |
| | | TTAGCTGGGTTCGTCAGGCACCTGGTCAAGGTCTGGAATGGATGGGTGGT | |
| | | ATTATTCCGATTTTTGGCACCGCAAATTATGCCCAGAAATTTCAGGGTCG | |
| | | TGTTACCATTACCGCAGATGAAAGCACCAGCACCGCATATATGGAACTGA | |
| | | GCAGCCTGCGTAGCGAAGATACGGCCGTCTATTATTGTGCGCGCCATTCT | |
| | | ACTCCATCTTTTCTGCAGTACTGGGGCCAGGGCACCCTGGTTACTGTCTC | |
| | | GAGC | |
| | VL (DNA) | AGCTATGAACTGACCCAGCCTCCGAGCGTTAGCGTTAGTCCGGGTCAGAC | 500 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCGCAGTTTACCCAGCACATGCATCTGCACGTTGGGTGTTC | |
| | | GGCGGTGGTACCAAGTTAACCGTGCTG | |
| C-012 | H-CDR1 | SYAMS | 501 |
| | H-CDR2 | AISGSGGSTYYADSVKG | 502 |
| | H-CDR3 | HWVSYGPFDY | 503 |
| | VH (aa) | EVQLLESGGGLVQPGGSLRLSCAASGFTFSSYAMSWVRQAPGKGLEWVSA | 504 |
| | | ISGSGGSTYYADSVKGRFTISRDNSKNTLYLQMNSLRAEDTAVYYCARHW | |
| | | VSYGPFDYWGQGTLVTVSS | |
| | L-CDR1 | RASQSISSYLN | 505 |
| | L-CDR2 | AASSLQS | 506 |
| | L-CDR3 | QQSHQSPPIT | 507 |
| | VL (aa) | DIQMTQSPSSLSASVGDRVTITCRASQSISSYLNWYQQKPGKAPKLLIYA | 508 |
| | | ASSLQSGVPSRFSGSGSGTDFTLTISSLQPEDFATYYCQQSHQSPPITFG | |
| | | QGTKVEIK | |
| | VH (DNA) | GAAGTTCAGCTGCTGGAAAGCGGTGGTGGTCTGGTTCAGCCTGGTGGTAG | 509 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTATATTACTGTGCGAGACATTGG | |
| | | GTCAGCTATGGCCCTTTTGACTACTGGGGCCAGGGCACCCTGGTCACCGT | |
| | | CTCGAGC | |
| | VL (DNA) | GATATTCAGATGACCCAGAGTCCGAGCAGCCTGAGCGCAAGCGTTGGTGA | 510 |
| | | TCGTGTTACCATTACCTGTCGTGCAAGCCAGAGCATTAGCAGCTATCTGA | |
| | | ATTGGTATCAGCAGAAACCGGGTAAAGCACCGAAACTGCTGATTTATGCA | |
| | | GCAAGCAGCCTGCAGAGCGGTGTTCCGAGCCGTTTTAGCGGATCCGGTAG | |
| | | CGGCACCGATTTTACCCTGACCATTAGCAGTCTGCAGCCGGAAGACTTTG | |
| | | CCACCTATTATTGCCAGCAGTCTCATCAGTCTCCGCCGATCACTTTCGGC | |
| | | CAGGGTACCAAAGTGGAAATTAAG | |
| C-013 | H-CDR1 | NAWMS | 511 |
| | H-CDR2 | RIKSKTDGGTTDYAAPVKG | 512 |
| | H-CDR3 | IEGSHGFDY | 513 |
| | VH (aa) | EVQLVESGGGLVKPGGSLRLSCAASGFTFSNAWMSWVRQAPGKGLEWVGR | 514 |
| | | IKSKTDGGTTDYAAPVKGRFTISRDDSKNTLYLQMNSLKTEDTAVYYCAR | |
| | | IEGSHGFDYWGQGTLVTVSS | |
| | L-CDR1 | SGDKLGDKYAS | 515 |
| | L-CDR2 | QDSKRPS | 516 |
| | L-CDR3 | ASYLHTPKQFV | 517 |
| | VL (aa) | SYELTQPPSVSVSPGQTASITCSGDKLGDKYASWYQQKPGQSPVLVIYQD | 518 |
| | | SKRPSGIPERFSGSNSGNTATLTISGTQAEDEADYYCASYLHTPKQFVFG | |
| | | GGTKLTVL | |
| | VH (DNA) | GAAGTTCAGCTGGTTGAAAGCGGTGGTGGTCTGGTTAAACCTGGTGGTAG | 519 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | ATCGAAGGTTCTCATGGTTTTGATTACTGGGGCCAGGGCACCCTGGTTAC | |
| | | TGTCTCGAGC | |
| | VL (DNA) | AGCTATGAACTGACCCAGCCTCCGAGCGTTAGCGTTAGTCCGGGTCAGAC | 520 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCGCATCTTACCTGCATACTCCAAAACAGTTTGTGTTCGGC | |
| | | GGTGGTACCAAGTTAACCGTGCTG | |
| C-014 | H-CDR1 | NAWMS | 521 |
| | H-CDR2 | RIKSKTDGGTTDYAAPVKG | 522 |
| | H-CDR3 | LGSYEGFDY | 523 |
| | VH (aa) | EVQLVESGGGLVKPGGSLRLSCAASGFTFSNAWMSWVRQAPGKGLEWVGR | 524 |
| | | IKSKTDGGTTDYAAPVKGRFTISRDDSKNTLYLQMNSLKTEDTAVYYCAR | |
| | | LGSYEGFDYWGQGTLVTVSS | |
| | L-CDR1 | SGDKLGDKYAS | 525 |
| | L-CDR2 | QDSKRPS | 526 |
| | L-CDR3 | STYTVTSSVW | 527 |
| | VL (aa) | SYELTQPPSVSVSPGQTASITCSGDKLGDKYASWYQQKPGQSPVLVIYQD | 528 |
| | | SKRPSGIPERFSGSNSGNTATLTISGTQAEDEADYYCSTYTVTSSVVVFG | |
| | | GGTKLTVL | |
| | VH (DNA) | GAAGTTCAGCTGGTTGAAAGCGGTGGTGGTCTGGTTAAACCTGGTGGTAG | 529 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTTCTTACGAAGGTTTTGATTACTGGGGCCAGGGCACCCTGGTTAC | |
| | | TGTCTCGAGC | |
| | VL (DNA) | AGCTATGAACTGACCCAGCCTCCGAGCGTTAGCGTTAGTCCGGGTCAGAC | 530 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCTCTACTTACACTGTTACTTCTTCTGTTGTTGTGTTCGGC | |
| | | GGTGGTACCAAGTTAACCGTGCTG | |
| C-015 | H-CDR1 | NAWMS | 531 |
| | H-CDR2 | RIKSKTDGGTTDYAAPVKG | 532 |
| | H-CDR3 | VAHGGYSGGLDP | 533 |
| | VH (aa) | EVQLVESGGGLVKPGGSLRLSCAASGFTFSNAWMSWVRQAPGKGLEWVGR | 534 |
| | | IKSKTDGGTTDYAAPVKGRFTISRDDSKNTLYLQMNSLKTEDTAVYYCAR | |
| | | VAHGGYSGGLDPWGQGTLVTVSS | |
| | L-CDR1 | SGDKLGDKYAS | 535 |
| | L-CDR2 | QDSKRPS | 536 |
| | L-CDR3 | SSTGTAHTLAV | 537 |
| | VL (aa) | SYELTQPPSVSVSPGQTASITCSGDKLGDKYASWYQQKPGQSPVLVIYQD | 538 |
| | | SKRPSGIPERFSGSNSGNTATLTISGTQAEDEADYYCSSTGTAHTLAVFG | |
| | | GGTKLTVL | |
| | VH (DNA) | GAAGTTCAGCTGGTTGAAAGCGGTGGTGGTCTGGTTAAACCTGGTGGTAG | 539 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | GTTGCACATGGTGGTTACTCTGGTGGTCTGGATCCATGGGGCCAGGGCAC | |
| | | CCTGGTTACTGTCTCGAGC | |
| | VL (DNA) | AGCTATGAACTGACCCAGCCTCCGAGCGTTAGCGTTAGTCCGGGTCAGAC | 540 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCTCTTCTACTGGTACTGCACATACTCTGGCAGTGTTCGGC | |
| | | GGTGGTACCAAGTTAACCGTGCTG | |
| C-016 | H-CDR1 | SYAMS | 541 |
| | H-CDR2 | AISGSGGSTYYADSVKG | 542 |
| | H-CDR3 | VYRAFDY | 543 |
| | VH (aa) | EVQLLESGGGLVQPGGSLRLSCAASGFTFSSYAMSWVRQAPGKGLEWVSA | 544 |
| | | ISGSGGSTYYADSVKGRFTISRDNSKNTLYLQMNSLRAEDTAVYYCARVY | |
| | | RAFDYWGQGTLVTVSS | |
| | L-CDR1 | SGSSSNIGNNYVS | 545 |
| | L-CDR2 | DNNKRPS | 546 |
| | L-CDR3 | HLYTEAESHWV | 547 |
| | VL (aa) | ASVLTQPPSVSAAPGQKVTISCSGSSSNIGNNYVSWYQQLPGTAPKLLIY | 548 |
| | | DNNKRPSGIPDRFSGSKSGTSATLGITGLQAEDEADYYCHLYTEAESHWV | |
| | | FGGGTKLTVL | |
| | VH (DNA) | GAAGTTCAGCTGCTGGAAAGCGGTGGTGGTCTGGTTCAGCCTGGTGGTAG | 549 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTCTATTATTGTGCGCGCGTTTAC | |
| | | CGTGCATTTGATTACTGGGGCCAGGGCACCCTGGTTACTGTCTCGAGC | |
| | VL (DNA) | GCGAGCGTTCTGACCCAGCCTCCGAGCGTTAGCGCAGCACCGGGTCAGAA | 550 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCATCTGTACACTGAAGCAGAATCTCATTGGGTG | |
| | | TTCGGCGGTGGTACCAAGTTAACCGTGCTG | |
| C-017 | H-CDR1 | SYAMS | 551 |
| | H-CDR2 | AISGSGGSTYYADSVKG | 552 |
| | H-CDR3 | DGSGPTLDL | 553 |
| | VH (aa) | EVQLLESGGGLVQPGGSLRLSCAASGFTFSSYAMSWVRQAPGKGLEWVSA | 554 |
| | | ISGSGGSTYYADSVKGRFTISRDNSKNTLYLQMNSLRAEDTAVYYCARDG | |
| | | SGPTLDLWGRGTLVTVSS | |
| | L-CDR1 | RASQSISSYLN | 555 |
| | L-CDR2 | AASSLQS | 556 |
| | L-CDR3 | QQHRYIPPWT | 557 |
| | VL (aa) | DIQMTQSPSSLSASVGDRVTITCRASQSISSYLNWYQQKPGKAPKLLIYA | 558 |
| | | ASSLQSGVPSRFSGSGSGTDFTLTISSLQPEDFATYYCQQHRYIPPWTFG | |
| | | QGTKVEIK | |
| | VH (DNA) | GAAGTTCAGCTGCTGGAAAGCGGTGGTGGTCTGGTTCAGCCTGGTGGTAG | 559 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTGTATTACTGTGCGAGAGATGGT | |
| | | TCCGGCCCCACTCTCGATCTCTGGGGCCGTGGCACCCTGGTCACTGTCTC | |
| | | GAGC | |
| | VL (DNA) | GATATTCAGATGACCCAGAGTCCGAGCAGCCTGAGCGCAAGCGTTGGTGA | 560 |
| | | TCGTGTTACCATTACCTGTCGTGCAAGCCAGAGCATTAGCAGCTATCTGA | |
| | | ATTGGTATCAGCAGAAACCGGGTAAAGCACCGAAACTGCTGATTTATGCA | |
| | | GCAAGCAGCCTGCAGAGCGGTGTTCCGAGCCGTTTTAGCGGATCCGGTAG | |
| | | CGGCACCGATTTTACCCTGACCATTAGCAGTCTGCAGCCGGAAGACTTTG | |
| | | CCACCTATTATTGCCAGCAGCATCGTTACATCCCGCCGTGGACTTTCGGC | |
| | | CAGGGTACCAAAGTGGAAATTAAG | |
| C-018 | H-CDR1 | SYAMS | 561 |
| | H-CDR2 | AISGSGGSTYYADSVKG | 562 |
| | H-CDR3 | STPGYYYVHYGFDI | 563 |
| | VH (aa) | EVQLLESGGGLVQPGGSLRLSCAASGFTFSSYAMSWVRQAPGKGLEWVSA | 564 |
| | | ISGSGGSTYYADSVKGRFTISRDNSKNTLYLQMNSLRAEDTAVYYCARST | |
| | | PGYYYVHYGFDIWGQGTLVTVSS | |
| | L-CDR1 | SGDKLGDKYAS | 565 |
| | L-CDR2 | QDSKRPS | 566 |
| | L-CDR3 | QAWSTSTHSWV | 567 |
| | VL (aa) | SYELTQPPSVSVSPGQTASITCSGDKLGDKYASWYQQKPGQSPVLVIYQD | 568 |
| | | SKRPSGIPERFSGSNSGNTATLTISGTQAEDEADYYCQAWSTSTHSWVFG | |
| | | GGTKLTVL | |
| | VH (DNA) | GAAGTTCAGCTGCTGGAAAGCGGTGGTGGTCTGGTTCAGCCTGGTGGTAG | 569 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTCTATTATTGTGCGCGCTCTACT | |
| | | CCAGGTTACTACTACGTTCATTACGGTTTTGATATCTGGGGCCAGGGCAC | |
| | | CCTGGTTACTGTCTCGAGC | |
| | VL (DNA) | AGCTATGAACTGACCCAGCCTCCGAGCGTTAGCGTTAGTCCGGGTCAGAC | 570 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCAGGCATGGTCTACTTCTACTCATTCTTGGGTGTTCGGC | |
| | | GGTGGTACCAAGTTAACCGTGCTG | |
| C-019 | H-CDR1 | SYAMS | 571 |
| | H-CDR2 | AISGSGGSTYYADSVKG | 572 |
| | H-CDR3 | GSPYWGVFDY | 573 |
| | VH (aa) | EVQLLESGGGLVQPGGSLRLSCAASGFTFSSYAMSWVRQAPGKGLEWVSA | 574 |
| | | ISGSGGSTYYADSVKGRFTISRDNSKNTLYLQMNSLRAEDTAVYYCARGS | |
| | | PYVVGVFDYWGQGTLVTVSS | |
| | L-CDR1 | RASQSISSYLN | 575 |
| | L-CDR2 | AASSLQS | 576 |
| | L-CDR3 | QQWQHEPPYT | 577 |
| | VL (aa) | DIQMTQSPSSLSASVGDRVTITCRASQSISSYLNWYQQKPGKAPKLLIYA | 578 |
| | | ASSLQSGVPSRFSGSGSGTDFTLTISSLQPEDFATYYCQQWQHEPPYTFG | |
| | | QGTKVEIK | |
| | VH (DNA) | GAAGTTCAGCTGCTGGAAAGCGGTGGTGGTCTGGTTCAGCCTGGTGGTAG | 579 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTCTATTATTGTGCGCGCGGTTCT | |
| | | CCATACGTTGTTGGTGTTTTTGATTACTGGGGCCAGGGCACCCTGGTTAC | |
| | | TGTCTCGAGC | |
| | VL (DNA) | GATATTCAGATGACCCAGAGTCCGAGCAGCCTGAGCGCAAGCGTTGGTGA | 580 |
| | | TCGTGTTACCATTACCTGTCGTGCAAGCCAGAGCATTAGCAGCTATCTGA | |
| | | ATTGGTATCAGCAGAAACCGGGTAAAGCACCGAAACTGCTGATTTATGCA | |
| | | GCAAGCAGCCTGCAGAGCGGTGTTCCGAGCCGTTTTAGCGGATCCGGTAG | |
| | | CGGCACCGATTTTACCCTGACCATTAGCAGTCTGCAGCCGGAAGACTTTG | |
| | | CCACCTATTATTGCCAGCAGTGGCAGCATGAACCGCCGTACACTTTCGGC | |
| | | CAGGGTACCAAAGTGGAAATTAAG | |
| C-020 | H-CDR1 | NAWMS | 581 |
| | H-CDR2 | RIKSKTDGGTTDYAAPVKG | 582 |
| | H-CDR3 | LHIYGPFDY | 583 |
| | VH (aa) | EVQLVESGGGLVKPGGSLRLSCAASGFTFSNAWMSWVRQAPGKGLEWVGR | 584 |
| | | IKSKTDGGTTDYAAPVKGRFTISRDDSKNTLYLQMNSLKTEDTAVYYCAR | |
| | | LHIYGPFDYWGQGTLVTVSS | |
| | L-CDR1 | SGDKLGDKYAS | 585 |
| | L-CDR2 | QDSKRPS | 586 |
| | L-CDR3 | LSYLARSGSW | 587 |
| | VL (aa) | SYELTQPPSVSVSPGQTASITCSGDKLGDKYASWYQQKPGQSPVLVIYQD | 588 |
| | | SKRPSGIPERFSGSNSGNTATLTISGTQAEDEADYYCLSYLARSGSVVFG | |
| | | GGTKLTVL | |
| | VH (DNA) | GAAGTTCAGCTGGTTGAAAGCGGTGGTGGTCTGGTTAAACCTGGTGGTAG | 589 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGCATATCTACGGTCCATTTGATTACTGGGGCCAGGGCACCCTGGTTAC | |
| | | TGTCTCGAGC | |
| | VL (DNA) | AGCTATGAACTGACCCAGCCTCCGAGCGTTAGCGTTAGTCCGGGTCAGAC | 590 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCTGTCTTACCTGGCACGTTCTGGTTCTGTTGTGTTCGGC | |
| | | GGTGGTACCAAGTTAACCGTGCTG | |
| C-021 | H-CDR1 | SYAMS | 591 |
| | H-CDR2 | AISGSGGSTYYADSVKG | 592 |
| | H-CDR3 | HEVFGTSSGYHLYAFDI | 593 |
| | VH (aa) | EVQLLESGGGLVQPGGSLRLSCAASGFTFSSYAMSWVRQAPGKGLEWVSA | 594 |
| | | ISGSGGSTYYADSVKGRFTISRDNSKNTLYLQMNSLRAEDTAVYYCARHE | |
| | | VFGTSSGYHLYAFDIWGQGTLVTVSS | |
| | L-CDR1 | RASQSISSYLN | 595 |
| | L-CDR2 | AASSLQS | 596 |
| | L-CDR3 | QQWSGLPLT | 597 |
| | VL (aa) | DIQMTQSPSSLSASVGDRVTITCRASQSISSYLNWYQQKPGKAPKLLIYA | 598 |
| | | ASSLQSGVPSRFSGSGSGTDFTLTISSLQPEDFATYYCQQWSGLPLTFGQ | |
| | | GTKVEIK | |
| | VH (DNA) | GAAGTTCAGCTGCTGGAAAGCGGTGGTGGTCTGGTTCAGCCTGGTGGTAG | 599 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTCTATTATTGTGCGCGCCATGAA | |
| | | GTTTTTGGTACTTCTTCTGGTTACCATCTGTACGCATTTGATATCTGGGG | |
| | | CCAGGGCACCCTGGTTACTGTCTCGAGC | |
| | VL (DNA) | GATATTCAGATGACCCAGAGTCCGAGCAGCCTGAGCGCAAGCGTTGGTGA | 600 |
| | | TCGTGTTACCATTACCTGTCGTGCAAGCCAGAGCATTAGCAGCTATCTGA | |
| | | ATTGGTATCAGCAGAAACCGGGTAAAGCACCGAAACTGCTGATTTATGCA | |
| | | GCAAGCAGCCTGCAGAGCGGTGTTCCGAGCCGTTTTAGCGGATCCGGTAG | |
| | | CGGCACCGATTTTACCCTGACCATTAGCAGTCTGCAGCCGGAAGACTTTG | |
| | | CCACCTATTATTGCCAGCAGTGGTCTGGTCTGCCGCTGACTTTCGGCCAG | |
| | | GGTACCAAAGTGGAAATTAAG | |
| C-022 | H-CDR1 | SYAMS | 601 |
| | H-CDR2 | AISGSGGSTYYADSVKG | 602 |
| | H-CDR3 | MAAGASWGTFDY | 603 |
| | VH (aa) | EVQLLESGGGLVQPGGSLRLSCAASGFTFSSYAMSWVRQAPGKGLEWVSA | 604 |
| | | ISGSGGSTYYADSVKGRFTISRDNSKNTLYLQMNSLRAEDTAAYYCARMA | |
| | | AGASWGTFDYWSQGTLVTVSS | |
| | L-CDR1 | RASQSISSYLN | 605 |
| | L-CDR2 | AASSLQS | 606 |
| | L-CDR3 | QQRSGSPLT | 607 |
| | VL (aa) | DIQMTQSPSSLSASVGDRVTITCRASQSISSYLNWYQQKPGKAPKLLIYA | 608 |
| | | ASSLQSGVPSRFSGSGSGTDFTLTISSLQPEDFATYYCQQRSGSPLTFGQ | |
| | | GTKVEIK | |
| | VH (DNA) | GAAGTTCAGCTGCTGGAAAGCGGTGGTGGTCTGGTTCAGCCTGGTGGTAG | 609 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGCATATTACTGTGCACGGATGGCT | |
| | | GCAGGCGCCAGTTGGGGGACCTTCGACTACTGGAGCCAGGGAACCCTGGT | |
| | | CACCGTCTCGAGC | |
| | VL (DNA) | GATATTCAGATGACCCAGAGTCCGAGCAGCCTGAGCGCAAGCGTTGGTGA | 610 |
| | | TCGTGTTACCATTACCTGTCGTGCAAGCCAGAGCATTAGCAGCTATCTGA | |
| | | ATTGGTATCAGCAGAAACCGGGTAAAGCACCGAAACTGCTGATTTATGCA | |
| | | GCAAGCAGCCTGCAGAGCGGTGTTCCGAGCCGTTTTAGCGGATCCGGTAG | |
| | | CGGCACCGATTTTACCCTGACCATTAGCAGTCTGCAGCCGGAAGACTTTG | |
| | | CCACCTATTATTGCCAGCAGCGTTCTGGTTCTCCGCTGACTTTCGGCCAG | |
| | | GGTAGCAAAGTGGAAATTAAG | |
| C-023 | H-CDR1 | NAWMS | 611 |
| | H-CDR2 | RIKSKTDGGTTDYAAPVKG | 612 |
| | H-CDR3 | LGVFSGFDY | 613 |
| | VH (aa) | EVQLVESGGGLVKPGGSLRLSCAASGFTFSNAWMSWVRQAPGKGLEWVGR | 614 |
| | | IKSKTDGGTTDYAAPVKGRFTISRDDSKNTLYLQMNSLKTEDTAVYYCAR | |
| | | LGVFSGFDYWGQGTLVTVSS | |
| | L-CDR1 | SGDKLGDKYAS | 615 |
| | L-CDR2 | QDSKRPS | 616 |
| | L-CDR3 | HTWTHHSLAVV | 617 |
| | VL (aa) | SYELTQPPSVSVSPGQTASITCSGDKLGDKYASWYQQKPGQSPVLVIYQD | 618 |
| | | SKRPSGIPERFSGSNSGNTATLTISGTQAEDEADYYCHTWTHHSLAVVFG | |
| | | GGTKLTVL | |
| | VH (DNA) | GAAGTTCAGCTGGTTGAAAGCGGTGGTGGTCTGGTTAAACCTGGTGGTAG | 619 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTGTTTTTTCTGGTTTTGATTACTGGGGCCAGGGCACCCTGGTTAC | |
| | | TGTCTCGAGC | |
| | VL (DNA) | AGCTATGAACTGACCCAGCCTCCGAGCGTTAGCGTTAGTCCGGGTCAGAC | 620 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATACTTGGACTCATCATTCTCTGGCAGTTGTGTTCGGC | |
| | | GGTGGTACCAAGTTAACCGTGCTG | |
| C-024 | H-CDR1 | SYAMS | 621 |
| | H-CDR2 | AISGSGGSTYYADSVKG | 622 |
| | H-CDR3 | HEYLGFYFDV | 623 |
| | VH (aa) | EVQLLESGGGLVQPGGSLRLSCAASGFTFSSYAMSWVRQAPGKGLEWVSA | 624 |
| | | ISGSGGSTYYADSVKGRFTISRDNSKNTLYLQMNSLRAEDTAVYYCARHE | |
| | | YLGFYFDVWGQGTLVTVSS | |
| | L-CDR1 | SGDKLGDKYAS | 625 |
| | L-CDR2 | QDSKRPS | 626 |
| | L-CDR3 | LASHRLTLNYV | 627 |
| | VL (aa) | SYELTQPPSVSVSPGQTASITCSGDKLGDKYASWYQQKPGQSPVLVIYQD | 628 |
| | | SKRPSGIPERFSGSNSGNTATLTISGTQAEDEADYYCLASHRLTLNYVFG | |
| | | GGTKLTVL | |
| | VH (DNA) | GAAGTTCAGCTGCTGGAAAGCGGTGGTGGTCTGGTTCAGCCTGGTGGTAG | 629 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTCTATTATTGTGCGCGCCATGAA | |
| | | TACCTGGGTTTTTACTTTGATGTTTGGGGCCAGGGCACCCTGGTTACTGT | |
| | | CTCGAGC | |
| | VL (DNA) | AGCTATGAACTGACCCAGCCTCCGAGCGTTAGCGTTAGTCCGGGTCAGAC | 630 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCTGGCATCTCATCGTCTGACTCTGAACTACGTGTTCGGC | |
| | | GGTGGTACCAAGTTAACCGTGCTG | |
| C-025 | H-CDR1 | SYAMS | 631 |
| | H-CDR2 | AISGSGGSTYYADSVKG | 632 |
| | H-CDR3 | HYTVGVYVYEYFDY | 633 |
| | VH (aa) | EVQLLESGGGLVQPGGSLRLSCAASGFTFSSYAMSWVRQAPGKGLEWVSA | 634 |
| | | ISGSGGSTYYADSVKGRFTISRDNSKNTLYLQMNSLRAEDTAVYYCARHY | |
| | | TVGVYVYEYFDYWGQGTLVTVSS | |
| | L-CDR1 | SGDKLGDKYAS | 635 |
| | L-CDR2 | QDSKRPS | 636 |
| | L-CDR3 | QSYATAGFV | 637 |
| | VL (aa) | SYELTQPPSVSVSPGQTASITCSGDKLGDKYASWYQQKPGQSPVLVIYQD | 638 |
| | | SKRPSGIPERFSGSNSGNTATLTISGTQAEDEADYYCQSYATAGFVFGGG | |
| | | TKLTVL | |
| | VH (DNA) | GAAGTTCAGCTGCTGGAAAGCGGTGGTGGTCTGGTTCAGCCTGGTGGTAG | 639 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTCTATTATTGTGCGCGCCATTAC | |
| | | ACTGTTGGTGTTTACGTTTACGAATACTTTGATTACTGGGGCCAGGGCAC | |
| | | CCTGGTTACTGTCTCGAGC | |
| | VL (DNA) | AGCTATGAACTGACCCAGCCTCCGAGCGTTAGCGTTAGTCCGGGTCAGAC | 640 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCAGTCTTACGCAACTGCAGGTTTTGTGTTCGGCGGTGGT | |
| | | ACCAAGTTAACCGTGCTG | |
| C-026 | H-CDR1 | SYAMS | 641 |
| | H-CDR2 | AISGSGGSTYYADSVKG | 642 |
| | H-CDR3 | LHKVFEFYHYTYAFDY | 643 |
| | VH (aa) | EVQLLESGGGLVQPGGSLRLSCAASGFTFSSYAMSWVRQAPGKGLEWVSA | 644 |
| | | ISGSGGSTYYADSVKGRFTISRDNSKNTLYLQMNSLRAEDTAVYYCARLH | |
| | | KVFEFYHYTYAFDYWGQGTLVTVSS | |
| | L-CDR1 | SGSSSNIGNNYVS | 645 |
| | L-CDR2 | DNNKRPS | 646 |
| | L-CDR3 | ASYSSETSGWV | 647 |
| | VL (aa) | ASVLTQPPSVSAAPGQKVTISCSGSSSNIGNNYVSWYQQLPGTAPKLLIY | 648 |
| | | DNNKRPSGIPDRFSGSKSGTSATLGITGLQAEDEADYYCASYSSETSGWV | |
| | | FGGGTKLTVL | |
| | VH (DNA) | GAAGTTCAGCTGCTGGAAAGCGGTGGTGGTCTGGTTCAGCCTGGTGGTAG | 649 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTCTATTATTGTGCGCGCCTGCAT | |
| | | AAAGTTTTTGAATTTTACCATTACACTTACGCATTTGATTACTGGGGCCA | |
| | | GGGCACCCTGGTTACTGTCTCGAGC | |
| | VL (DNA) | GCGAGCGTTCTGACCCAGCCTCCGAGCGTTAGCGCAGCACCGGGTCAGAA | 650 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCGCATCTTACTCTTCTGAAACTTCTGGTTGGGTG | |
| | | TTCGGCGGCGGTACCAAGTTAACCGTGCTG | |
| C-027 | H-CDR1 | SYAIS | 651 |
| | H-CDR2 | GIIPIFGTANYAQKFQG | 652 |
| | H-CDR3 | ENIPSYYDSSGRQDAFDI | 653 |
| | VH (aa) | QVQLVQSGAEVKKPGSSVKVSCKASGGTFSSYAISWVRQAPGQGLEWMGG | 654 |
| | | IIPIFGTANYAQKFQGRVTITADESTSTAYMELSSLRSEDTAVYYCAREN | |
| | | IPSYYDSSGRQDAFDIWGQGTMVTVSS | |
| | L-CDR1 | SGDKLGDKYAS | 655 |
| | L-CDR2 | QDSKRPS | 656 |
| | L-CDR3 | QSYLHKSHGAV | 657 |
| | VL (aa) | SYELTQPPSVSVSPGQTASITCSGDKLGDKYASWYQQKPGQSPVLVIYQD | 658 |
| | | SKRPSGIPERFSGSNSGNTATLTISGTQAEDEADYYCQSYLHKSHGAVFG | |
| | | GGTKLTVL | |
| | VH (DNA) | CAGGTTCAGCTGGTTCAGAGCGGTGCAGAAGTTAAAAAACCGGGTAGCAG | 659 |
| | | CGTTAAAGTTAGCTGTAAAGCAAGCGGTGGCACCTTTAGCAGCTATGCAA | |
| | | TTAGCTGGGTTCGTCAGGCACCTGGTCAAGGTCTGGAATGGATGGGTGGT | |
| | | ATTATTCCGATTTTTGGCACCGCAAATTATGCCCAGAAATTTCAGGGTCG | |
| | | TGTTACCATTACCGCAGATGAAAGCACCAGCACCGCATATATGGAACTGA | |
| | | GCAGCCTGCGTAGCGAAGATACGGCCGTATATTACTGTGCGAGAGAAAAT | |
| | | ATCCCTAGTTACTATGATAGTAGTGGCCGCCAGGATGCTTTTGATATCTG | |
| | | GGGCCAAGGGACAATGGTCACCGTCTCGAGC | |
| | VL (DNA) | AGCTATGAACTGACCCAGCCTCCGAGCGTTAGCGTTAGTCCGGGTCAGAC | 660 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCAGTCTTACCTGCATAAATCTCATGGTGCAGTGTTCGGC | |
| | | GGTGGTACCAAGTTAACCGTGCTG | |
| C-028 | H-CDR1 | SYAMS | 661 |
| | H-CDR2 | AISGSGGSTYYADSVKG | 662 |
| | H-CDR3 | GQYVSGTYYSYGYWYFDL | 663 |
| | VH (aa) | EVQLLESGGGLVQPGGSLRLSCAASGFTFSSYAMSWVRQAPGKGLEWVSA | 664 |
| | | ISGSGGSTYYADSVKGRFTISRDNSKNTLYLQMNSLRAEDTAVYYCARGQ | |
| | | YVSGTYYSYGYWYFDLWGRGTLVTVSS | |
| | L-CDR1 | SGDKLGDKYAS | 665 |
| | L-CDR2 | QDSKRPS | 666 |
| | L-CDR3 | QVSRGHTSAGV | 667 |
| | VL (aa) | SYELTQPPSVSVSPGQTASITCSGDKLGDKYASWYQQKPGQSPVLVIYQD | 668 |
| | | SKRPSGIPERFSGSNSGNTATLTISGTQAEDEADYYCQVSRGHTSAGVFG | |
| | | GGTKLTVL | |
| | VH (DNA) | GAAGTTCAGCTGCTGGAAAGCGGTGGTGGTCTGGTTCAGCCTGGTGGTAG | 669 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTGTATTACTGTGCGAGAGGGCAA | |
| | | TATGTGTCGGGGACTTATTATTCCTACGGATACTGGTACTTCGATCTCTG | |
| | | GGGCCGTGGCACCCTGGTCACTGTCTCGAGC | |
| | VL (DNA) | AGCTATGAACTGACCCAGCCTCCGAGCGTTAGCGTTAGTCCGGGTCAGAC | 670 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCAGGTTTCTCGTGGTCATACTTCTGCAGGTGTGTTCGGC | |
| | | GGTGGTACCAAGTTAACCGTGCTG | |
| C-029 | H-CDR1 | SYAMS | 671 |
| | H-CDR2 | AISGSGGSTYYADSVKG | 672 |
| | H-CDR3 | HHGHGIYVHYYLDY | 673 |
| | VH (aa) | EVQLLESGGGLVQPGGSLRLSCAASGFTFSSYAMSWVRQAPGKGLEWVSA | 674 |
| | | ISGSGGSTYYADSVKGRFTISRDNSKNTLYLQMNSLRAEDTAVYYCARHH | |
| | | GHGIYVHYYLDYWGQGTLVTVSS | |
| | L-CDR1 | SGDKLGDKYAS | 675 |
| | L-CDR2 | QDSKRPS | 676 |
| | L-CDR3 | QTWAGTRLVV | 677 |
| | VL (aa) | SYELTQPPSVSVSPGQTASITCSGDKLGDKYASWYQQKPGQSPVLVIYQD | 678 |
| | | SKRPSGIPERFSGSNSGNTATLTISGTQAEDEADYYCQTWAGTRLVVFGG | |
| | | GTKLTVL | |
| | VH (DNA) | GAAGTTCAGCTGCTGGAAAGCGGTGGTGGTCTGGTTCAGCCTGGTGGTAG | 679 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTCTATTATTGTGCGCGCCATCAT | |
| | | GGTCATGGTATCTACGTTCATTACTACCTGGATTACTGGGGCCAGGGCAC | |
| | | CCTGGTTACTGTCTCGAGC | |
| | VL (DNA) | AGCTATGAACTGACCCAGCCTCCGAGCGTTAGCGTTAGTCCGGGTCAGAC | 680 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCAGACTTGGGCAGGTACTCGTCTGGTTGTGTTCGGCGGT | |
| | | GGTACCAAGTTAACCGTGCTG | |

Analysis of these sequences shows that the heavy-chain CDRs of ABPs C-003 and C-004 are the same, but have distinct light chain CDRs, that align as show in FIG. 24.

TABLE 13.1B:
Germline of VH and VLfor ABPs of this Example.

| Antibody | Germline of VH | Germline of VL |
|---|---|---|
| C-001 | IGHV3-23D\|IGHJ4*02 | IGLV1-51\|IGLJ3*02 |
| C-002 | IGHV3-23D\|IGHJ3*02 | IGLV1-51\|IGLJ3*02 |
| C-003 | IGHV3-23D\|IGHJ3*02 | IGLV1-51\|IGLJ3*02 |
| C-004 | IGHV3-23D\|IGHJ3*02 | IGLV1-51\|IGLJ3*02 |
| C-005 | IGHV3-15\|IGHJ4*02 | IGLV3-1\|IGLJ3*02 |
| C-006 | IGHV3-23D\|IGHJ4*02 | IGKV1D-39\|IGKJ1*01 |
| C-007 | IGHV3-23D\|IGHJ4*02 | IGLV1-51\|IGLJ3*02 |
| C-008 | IGHV3-23D\|IGHJ4*02 | IGLV1-51\|IGLJ3*02 |
| C-009 | IGHV3-23D\|IGHJ4*02 | IGLV3-1\|IGLJ3*02 |
| C-010 | IGHV1-69D\|IGHJ4*02 | IGLV3-1\|IGLJ3*02 |
| C-011 | IGHV1-69\|IGHJ4*02 | IGLV3-1\|IGLJ3*02 |
| C-012 | IGHV3-23D\|IGHJ4*02 | IGKV1-39\|IGKJ1*01 |
| C-013 | IGHV3-15\|IGHJ4*02 | IGLV3-1\|IGLJ3*02 |
| C-014 | IGHV3-15\|IGHJ4*02 | IGLV3-1\|IGLJ3*02 |
| C-015 | IGHV3-15\|IGHJ4*02 | IGLV3-1\|IGLJ3*02 |
| C-016 | IGHV3-23D\|IGHJ4*02 | IGLV1-51\|IGLJ3*02 |
| C-017 | IGHV3-23D\|IGHJ2*01 | IGKV1-39\|IGKJ1*01 |
| C-018 | IGHV3-23D\|IGHJ4*02 | IGLV3-1\|IGLJ3*02 |
| C-019 | IGHV3-23D\|IGHJ4*02 | IGKV1D-39\|IGKJ1*01 |
| C-020 | IGHV3-15\|IGHJ4*02 | IGLV3-1\|IGLJ3*02 |
| C-021 | IGHV3-23D\|IGHJ4*02 | IGKV1D-39\|IGKJ1*01 |
| C-022 | IGHV3-23D\|IGHJ4*02 | IGKV1D-39\|IGKJ1*01 |
| C-023 | IGHV3-15\|IGHJ4*02 | IGLV3-1\|IGLJ3*02 |
| C-024 | IGHV3-23D\|IGHJ4*02 | IGLV3-1\|IGLJ3*02 |
| C-025 | IGHV3-23D\|IGHJ4*02 | IGLV3-1\|IGLJ3*02 |
| C-026 | IGHV3-23D\|IGHJ4*02 | IGLV1-51\|IGLJ3*02 |
| C-027 | IGHV1-69D\|IGHJ3*02 | IGLV3-1\|IGLJ3*02 |
| C-028 | IGHV3-23D\|IGHJ2*01 | IGLV3-1\|IGLJ3*02 |
| C-029 | IGHV3-23\|IGHJ4*02 | IGLV3-1\|IGLJ3*02 |

TABLE 13.2
Binding signal (ELISA/A450) of binding of Fab-format ABPs of this
Example to various antigens and EC50 (nM) of binding (FACS) of
IgG-format ABPs of this Example to cell lines.

| | | Protein-binding (ELISA) | | | | Cell-binding | |
|---|---|---|---|---|---|---|---|
| | | | | | MDA- | (FACS) | |
| | | | Cynomolgus | | MB-231- | MC38- | CT26- |
| | Human | Mouse | monkey | | IGSF11 | IGSF11 | IGSF11 |
| Antibody | IGSF11 | IGSF11 | IGSF11 | Streptavidin | (human) | (murine) | (murine) |
| C-001 | 2-3 | 1-2 | 2-3 | <0.1 | <10 | 50-100 | 20-50 |
| C-002 | 1-2 | <1 | 1-2 | <0.1 | | | |
| C-003 | 2-3 | 1-2 | 2-3 | 0.1-0.2 | 10-15 | <20 | <20 |
| C-004 | <1 | <1 | <1 | <0.1 | | | |
| C-005 | 2-3 | 2-3 | 2-3 | <0.1 | <10 | <20 | <20 |
| C-006 | 1-2 | <1 | <1 | 0.1-0.2 | | | |
| C-007 | 2-3 | 1-2 | 2-3 | <0.1 | <10 | <20 | 20-50 |
| C-008 | 2-3 | 2-3 | 2-3 | <0.1 | | | |
| C-009 | 1-2 | <1 | 1-2 | <0.1 | | | |
| C-010 | 1-2 | <1 | <1 | <0.1 | | | |
| C-011 | 2-3 | 1-2 | 2-3 | 0.1-0.2 | | | |
| C-012 | 1-2 | <1 | <1 | 0.1-0.2 | | | |
| C-013 | <1 | <1 | 1-2 | <0.1 | | | |
| C-014 | 1-2 | 1-2 | 1-2 | 0.1-0.2 | | | |
| C-015 | 1-2 | <1 | 1-2 | <0.1 | | | |
| C-016 | <1 | <1 | <1 | <0.1 | 15-25 | 50-100 | 20-50 |
| C-017 | 1-2 | <1 | 1-2 | 0.2-1 | 15-25 | 50-100 | 20-50 |
| C-018 | <1 | <1 | <1 | <0.1 | | | |
| C-019 | <1 | <1 | <1 | <0.1 | | | |
| C-020 | 1-2 | <1 | 1-2 | 0.1-0.2 | | | |
| C-021 | 1-2 | <1 | <1 | <0.1 | | | |
| C-022 | 2-3 | 1-2 | 1-2 | <0.1 | | | |
| C-023 | 1-2 | <1 | 1-2 | <0.1 | | | |
| C-024 | 1-2 | <1 | 1-2 | 0.1-0.2 | | | |
| C-025 | 2-3 | 1-2 | 2-3 | 0.1-0.2 | | | |
| C-026 | 2-3 | <1 | 2-3 | <0.1 | | | |
| C-027 | <1 | <1 | <1 | <0.1 | | | |
| C-028 | 1-2 | <1 | <1 | 0.2-1 | | | |
| C-029 | 2-3 | 2-3 | 2-3 | 0.2-1 | | | |

Further ABPs of the invention that bind to the IgC2 domain of IGSF11 were affinity maturated as described below, and particularly high affinity ABPs D-114 and D-222 were identified.

Maturation Library Construction

Affinity-improved IGSF11 ABPs were selected by phage display from antibody gene libraries based on the parental V gene sequence with diversified CDR-H1/H2 and CDR-L3, respectively. For each parental sequence, two diversified libraries were constructed keeping the light chain constant and diversifying CDR-H1 and CDR-H2 and keeping the heavy chain constant and diversifying CDR-L3. Each library contained more than 10e8 derivatives of the respective parental sequence.

Selection of Affinity-Improved Binders

To select higher affinity binders, optimized selection conditions were applied. Briefly, the diversified antibody phage libraries were blocked with 2× ChemiBLOCKER (Merck Millipore), in the first panning round the biotinylated recombinant protein was added at a concentration of 50 nM and incubated for 1 h at room temperature. Antibody phage bound to recombinant IGSF11 were separated using Streptavidin magnetic beads (Dynabeads M-280, ThermoFisher) and washed with PBST. The antibody phage were eluted using 10 ug/mL Trypsin and used to infect mid-logarithmic E. coli TG1 for phage amplification.

Panning round two and three were performed equivalently to panning round one with the following modifications to increase the selection pressure for higher affinity: The concentration of biotinylated IGSF11 recombinant protein was limited (5 and 0.5 nM in panning round two and 0.5 and 0.05 nM in panning round three) and incubation at room temperature was prolonged (2h in panning round two and 5h in panning round three). After capturing the biotinylated antigen with the bound antibody phage on Streptavidin magnetic beads (Dynabeads M-280, ThermoFisher), an initial washing step with PBST was performed. To increase the stringency of the washing and select for slower dissociation rates, the beads were suspended in 500 uL of PBST containing 500 nM of non-biotinylated IGSF11 recombinant competitor protein and incubated between 5 and 20h at room temperature.

Enrichment of higher affinity binders and the optimal selection stringency was monitored by determining the phage titers in the selection output of each condition and panning round.

ELISA Screen

To identify affinity-improved and specific IGSF11 binders, monoclonal Fabs were expressed in E. coli after the second and third panning round. After bacterial lysis, the Fabs were tested for their binding properties and cross-reactivity profile on recombinant IGSF11 (human, mouse and cynomolgus monkey) by standard ELISA in a 1:200 dilution. Briefly, biotinylated recombinant IGSF11 from human, mouse, or cynomolgus monkey were immobilized at 1 ug/mL on a Streptavidin-coated 384-well Maxisorp plate. The surface was blocked with 2% (w/v) BSA in PBST. After three wash cycles with PBST, the bacterial lysates in 2% (w/v) BSA were applied to the immobilized IGSF11 and incubated for 1.5h. After removing all unbound antibodies by 3 wash cycles with PBST, bound Fab antibodies were detected with a goat anti-human Fab antibody conjugated with horseradish peroxidase. After three wash cycles with PBST the ELISA was developed with TMB substrate.

FACS Screen

ELISA-positive hits with desired cross-reactivity profile and improved binding over the parent clone were subsequently analyzed for their cell binding properties by standard multiplex flow cytometry. Briefly, MDA-MB-231 overexpressing human IGSF11 and wildtype MDA-MB-231 (not expressing IGSF11) were stained with different concentrations (500 nM and unstained) of CellTrace™ violet (Invitrogen) to perform multiplex flow cytometry analysis. The differently labeled cell lines were mixed in a 1:1 ratio and 30,000 cells were stained with E. coli lysates containing the monoclonal Fabs in 384 well format. Unbound antibodies were removed by washing the cells three time with FACS buffer. Bound antibodies were detected with a mouse anti-human Fab antibody conjugated with AlexaFluor647. Dead cells were excluded by 7-AAD or Zombie Green Dye (Biolegend) staining and the MFI of AlexaFluor647 was analyzed for the two differently CellTrace™ violet (Invitrogen) stained cell populations to determine specific binding to cellular expressed IGSF11.

Off-Rate Screen

The dissociation rates (off-rate) of the best cell binders were analyzed by biolayer interferometry (OctetRED96e) in a standard kinetic experiment using recombinant human and mouse IGSF11 protein. Briefly, biotinylated recombinant IGSF11 was immobilized on streptavidin coated biosensors. The sensors were dipped in E. coli lysates containing the monoclonal Fabs and antibodies were associated for 180s. Subsequently, the sensors were dipped into kinetics buffer to measure the dissociation for 240-300s. All sensorgrams were double referenced against kinetic buffer and unloaded streptavidin sensors to subtract sensor drifts and potential reference binding of the bacterial lysates and dissociation rates were fitted using a 1:1 binding model.

Recombination of Improved Heavy and Light Chains

Based on ELISA, FACS, off-rate and CDR sequence, those heavy chains and light chains with improved affinity were selected for each of the parent antibodies. The antibody V genes were amplified by PCR and recombined in a pool cloning approach using restriction enzyme independent DNA assembly. All unique heavy-light chain combinations were identified by DNA sequencing and expressed in E. coli as Fabs. Cell binding of the heavy-light chain combinations were analyzed by flow cytometry and off-rates were determined by biolayer interferometry as described before. Heavy-light chain combinations were converted into IgG format.

Affinity maturated antibodies of this Example are described in Table 13.3, showing for each such antibody the heavy chain and light chain CDR sequences and variable region sequences comprised in each such antibody as well as nucleic acid sequences encoding for such variable regions. The degree of binding of each such antibody to human, murine and cynomolgus monkey IGSF11 protein (and to irrelevant antigen), as determined by the ELISA, and to mouse IGSF11 protein expressed by cells, as determined by flow cytometry (FC), was determined generally as described above. All maturated mAbs exhibited highly specific binding to human, mouse and cynomolgus monkey IGSF11 ECD in ELISA (data not shown), and binding to IGSF11 protein expressed by cells is shown in Table 13.4, with better EC50 of cell binding compared to a parental ABP (C-005) which had an EC50 of about 2 nM to each cell line

TABLE 13.3
Amino acid sequences of CDR and variable regions of affinity maturated ABPs of this Example, as well
as nucleic acid sequences encoding variable regions of ABPs of this Example.

| | | | SEQ |
|---|---|---|---|
| | | Sequence | ID |
| Antibody | Region | 1.......10........20........30........40........50 | NO. |
| D-101 | H-CDR1 | FSALS | 681 |
| | H-CDR2 | AISYGGGSKYYADSVKG | 682 |
| | H-CDR3 | DSRDAYGVAFDL | 683 |
| | VH (aa) | EVQLLESGGGLVQPGGSLRLSCAASGFTFQFSALSWVRQAPGKGLEWVSA | 684 |
| | | ISYGGGSKYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 685 |
| | L-CDR2 | DNNKRPS | 686 |
| | L-CDR3 | LTWTGAGRIFV | 687 |
| | VL (aa) | ASVLTQPPSVSAAPGQKVTISCSGSSSNIGNNYVSWYQQLPGTAPKLLIY | 688 |
| | | DNNKRPSGIPDRFSGSKSGTSATLGITGLQAEDEADYYCLTWTGAGRIFV | |
| | | FGGGTKLTVL | |
| | VH (DNA) | GAAGTGCAGCTGCTTGAATCTGGCGGAGGACTGGTTCAGCCTGGCGGATC | 689 |
| | | TCTGAGACTGTCTTGTGCCGCCAGCGGCTTCACCTTCCAGTTTTCTGCCC | |
| | | TGAGCTGGGTCCGACAGGCCCCTGGAAAAGGACTGGAATGGGTGTCCGCC | |
| | | ATCTCTTACGGCGGAGGCAGCAAGTACTACGCCGACTCTGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCCTTTGATCTGTGGGGCCAGGGCACAATGGT | |
| | | CACAGTCTCGAGC | |
| | VL (DNA) | GCGAGCGTTCTGACCCAGCCTCCGAGCGTTAGCGCAGCACCGGGTCAGAA | 690 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGACTTGGACTGGTGCAGGTCGTATCTTTGTG | |
| | | TTCGGCGGTGGTACCAAGTTAACCGTGCTG | |
| D-102 | H-CDR1 | KYSLS | 691 |
| | H-CDR2 | AISYYGGGTLYADSVKG | 692 |
| | H-CDR3 | DSRDAYGVAFDL | 693 |
| | VH (aa) | EVQLLESGGGLVQPGGSLRLSCAASGFTFSKYSLSWVRQAPGKGLEWVSA | 694 |
| | | ISYYGGGTLYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 695 |
| | L-CDR2 | DNNKRPS | 696 |
| | L-CDR3 | LTWTGAGRIFV | 697 |
| | VL (aa) | ASVLTQPPSVSAAPGQKVTISCSGSSSNIGNNYVSWYQQLPGTAPKLLIY | 698 |
| | | DNNKRPSGIPDRFSGSKSGTSATLGITGLQAEDEADYYCLTWTGAGRIFV | |
| | | FGGGTKLTVL | |
| | VH (DNA) | GAAGTGCAGCTGCTTGAATCTGGCGGAGGACTGGTTCAACCTGGCGGCTC | 699 |
| | | TCTGAGACTGTCTTGTGCCGCCAGCGGCTTCACCTTTAGCAAGTATTCTC | |
| | | TGAGCTGGGTCCGACAGGCCCCTGGAAAAGGACTTGAATGGGTGTCCGCC | |
| | | ATCAGCTACTATGGCGGCGGAACACTTTACGCCGATAGCGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCATTTGATCTGTGGGGCCAGGGCACAATGGT | |
| | | CACAGTCTCGAGC | |
| | VL (DNA) | GCGAGCGTTCTGACCCAGCCTCCGAGCGTTAGCGCAGCACCGGGTCAGAA | 700 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGACTTGGACTGGTGCAGGTCGTATCTTTGTG | |
| | | TTCGGCGGTGGTACCAAGTTAACCGTGCTG | |
| D-103 | H-CDR1 | HSAIS | 701 |
| | H-CDR2 | AISYGGGSQYYADSVKG | 702 |
| | H-CDR3 | DSRDAYGVAFDL | 703 |
| | VH (aa) | EVQLLESGGGLVQPGGSLRLSCAASGFTFKHSAISWVRQAPGKGLEWVSA | 704 |
| | | ISYGGGSQYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 705 |
| | L-CDR2 | DNNKRPS | 706 |
| | L-CDR3 | LTWTGAGRIFV | 707 |
| | VL (aa) | ASVLTQPPSVSAAPGQKVTISCSGSSSNIGNNYVSWYQQLPGTAPKLLIY | 708 |
| | | DNNKRPSGIPDRFSGSKSGTSATLGITGLQAEDEADYYCLTWTGAGRIFV | |
| | | FGGGTKLTVL | |
| | VH (DNA) | GAAGTGCAGCTGCTTGAATCTGGCGGAGGACTGGTTCAACCTGGCGGCTC | 709 |
| | | TCTGAGACTGTCTTGTGCCGCCAGCGGCTTCACCTTTAAGCACAGCGCCA | |
| | | TTAGCTGGGTCCGACAGGCCCCTGGAAAAGGACTTGAATGGGTGTCCGCC | |
| | | ATCAGCTATGGCGGCGGAAGCCAATATTACGCCGATAGCGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCATTTGATCTGTGGGGCCAGGGCACAATGGT | |
| | | CACAGTCTCGAGC | |
| | VL (DNA) | GCGAGCGTTCTGACCCAGCCTCCGAGCGTTAGCGCAGCACCGGGTCAGAA | 710 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGACTTGGACTGGTGCAGGTCGTATCTTTGTG | |
| | | TTCGGCGGTGGTACCAAGTTAACCGTGCTG | |
| D-104 | H-CDR1 | FTTLS | 711 |
| | H-CDR2 | AISSAGGSSYYSDSVKG | 712 |
| | H-CDR3 | DSRDAYGVAFDL | 713 |
| | VH (aa) | EVQLLESGGGLVQPGGSLRLSCAASGFTFSFTTLSWVRQAPGKGLEWVSA | 714 |
| | | ISSAGGSSYYSDSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 715 |
| | L-CDR2 | DNNKRPS | 716 |
| | L-CDR3 | LTWTGAGRIFV | 717 |
| | VL (aa) | ASVLTQPPSVSAAPGQKVTISCSGSSSNIGNNYVSWYQQLPGTAPKLLIY | 718 |
| | | DNNKRPSGIPDRFSGSKSGTSATLGITGLQAEDEADYYCLTWTGAGRIFV | |
| | | FGGGTKLTVL | |
| | VH (DNA) | GAAGTGCAGCTGCTTGAATCTGGCGGAGGACTGGTTCAACCTGGCGGCTC | 719 |
| | | TCTGAGACTGTCTTGTGCCGCCAGCGGCTTCACCTTTAGCTTCACAACAC | |
| | | TGAGCTGGGTCCGACAGGCCCCTGGAAAAGGACTTGAATGGGTGTCCGCC | |
| | | ATCAGCTCTGCCGGCGGAAGCAGCTATTACAGCGATAGCGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCATTTGATCTGTGGGGCCAGGGCACAATGGT | |
| | | CACAGTCTCGAGC | |
| | VL (DNA) | GCGAGCGTTCTGACCCAGCCTCCGAGCGTTAGCGCAGCACCGGGTCAGAA | 720 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGACTTGGACTGGTGCAGGTCGTATCTTTGTG | |
| | | TTCGGCGGTGGTACCAAGTTAACCGTGCTG | |
| D-105 | H-CDR1 | TSSLS | 721 |
| | H-CDR2 | AASYSGSSQYYADSVKG | 722 |
| | H-CDR3 | DSRDAYGVAFDL | 723 |
| | VH (aa) | EVQLLESGGGLVQPGGSLRLSCAASGFTFTTSSLSWVRQAPGKGLEWVSA | 724 |
| | | ASYSGSSQYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 725 |
| | L-CDR2 | DNNKRPS | 726 |
| | L-CDR3 | LTWTGAGRIFV | 727 |
| | VL (aa) | ASVLTQPPSVSAAPGQKVTISCSGSSSNIGNNYVSWYQQLPGTAPKLLIY | 728 |
| | | DNNKRPSGIPDRFSGSKSGTSATLGITGLQAEDEADYYCLTWTGAGRIFV | |
| | | FGGGTKLTVL | |
| | VH (DNA) | GAAGTGCAGCTGCTTGAATCTGGCGGAGGACTGGTTCAACCTGGCGGCTC | 729 |
| | | TCTGAGACTGTCTTGTGCCGCCAGCGGCTTCACCTTTACAACAAGCAGCC | |
| | | TTAGCTGGGTCCGACAGGCCCCTGGAAAAGGACTTGAATGGGTGTCCGCC | |
| | | GCCAGCTACTCTGGCTCTAGCCAATATTACGCCGATAGCGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCATTTGATCTGTGGGGCCAGGGCACAATGGT | |
| | | CACAGTCTCGAGC | |
| | VL (DNA) | GCGAGCGTTCTGACCCAGCCTCCGAGCGTTAGCGCAGCACCGGGTCAGAA | 730 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGACTTGGACTGGTGCAGGTCGTATCTTTGTG | |
| | | TTCGGCGGTGGTACCAAGTTAACCGTGCTG | |
| D-106 | H-CDR1 | TSALS | 731 |
| | H-CDR2 | AISYAGSGQYYADSVKG | 732 |
| | H-CDR3 | DSRDAYGVAFDL | 733 |
| | VH (aa) | EVQLLESGGGLVQPGGSLRLSCAASGFTFSTSALSWVRQAPGKGLEWVSA | 734 |
| | | ISYAGSGQYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 735 |
| | L-CDR2 | DNNKRPS | 736 |
| | L-CDR3 | LTWTGAGRIFV | 737 |
| | VL (aa) | ASVLTQPPSVSAAPGQKVTISCSGSSSNIGNNYVSWYQQLPGTAPKLLIY | 738 |
| | | DNNKRPSGIPDRFSGSKSGTSATLGITGLQAEDEADYYCLTWTGAGRIFV | |
| | | FGGGTKLTVL | |
| | VH (DNA) | GAAGTGCAGCTGCTTGAATCTGGCGGAGGACTGGTTCAACCTGGCGGCTC | 739 |
| | | TCTGAGACTGTCTTGTGCCGCCAGCGGCTTCACCTTTAGCACAAGCGCCC | |
| | | TTAGCTGGGTCCGACAGGCCCCTGGAAAAGGACTTGAATGGGTGTCCGCC | |
| | | ATCAGCTATGCCGGCTCTGGCCAATATTACGCCGATAGCGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCATTTGATCTGTGGGGCCAGGGCACAATGGT | |
| | | CACAGTCTCGAGC | |
| | VL (DNA) | GCGAGCGTTCTGACCCAGCCTCCGAGCGTTAGCGCAGCACCGGGTCAGAA | 740 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGACTTGGACTGGTGCAGGTCGTATCTTTGTG | |
| | | TTCGGCGGTGGTACCAAGTTAACCGTGCTG | |
| D-107 | H-CDR1 | TSALS | 741 |
| | H-CDR2 | AISYGGGSHYYADSVKG | 742 |
| | H-CDR3 | DSRDAYGVAFDL | 743 |
| | VH (aa) | EVQLLESGGGLVQPGGSLRLSCAASGFTFATSALSWVRQAPGKGLEWVSA | 744 |
| | | ISYGGGSHYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 745 |
| | L-CDR2 | DNNKRPS | 746 |
| | L-CDR3 | LTWTGAGRIFV | 747 |
| | VL (aa) | ASVLTQPPSVSAAPGQKVTISCSGSSSNIGNNYVSWYQQLPGTAPKLLIY | 748 |
| | | DNNKRPSGIPDRFSGSKSGTSATLGITGLQAEDEADYYCLTWTGAGRIFV | |
| | | FGGGTKLTVL | |
| | VH (DNA) | GAAGTGCAGCTGCTTGAATCTGGCGGAGGACTGGTTCAACCTGGCGGCTC | 749 |
| | | TCTGAGACTGTCTTGTGCCGCCAGCGGCTTCACCTTTGCTACAAGCGCCC | |
| | | TTAGCTGGGTCCGACAGGCCCCTGGAAAAGGACTTGAATGGGTGTCCGCC | |
| | | ATCAGCTATGGCGGCGGAAGCCATTATTACGCCGATAGCGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCATTTGATCTGTGGGGCCAGGGCACAATGGT | |
| | | CACAGTCTCGAGC | |
| | VL (DNA) | GCGAGCGTTCTGACCCAGCCTCCGAGCGTTAGCGCAGCACCGGGTCAGAA | 750 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGACTTGGACTGGTGCAGGTCGTATCTTTGTG | |
| | | TTCGGCGGTGGTACCAAGTTAACCGTGCTG | |
| D-108 | H-CDR1 | FYSLS | 751 |
| | H-CDR2 | AISGGGGGSYYADSVKG | 752 |
| | H-CDR3 | DSRDAYGVAFDL | 753 |
| | VH (aa) | EVQLLESGGGLVQPGGSLRLSCAASGFTFSFYSLSWVRQAPGKGLEWVSA | 754 |
| | | ISGGGGGSYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 755 |
| | L-CDR2 | DNNKRPS | 756 |
| | L-CDR3 | LTWTGAGRIFV | 757 |
| | VL (aa) | ASVLTQPPSVSAAPGQKVTISCSGSSSNIGNNYVSWYQQLPGTAPKLLIY | 758 |
| | | DNNKRPSGIPDRFSGSKSGTSATLGITGLQAEDEADYYCLTWTGAGRIFV | |
| | | FGGGTKLTVL | |
| | VH (DNA) | GAAGTGCAGCTGCTTGAATCTGGCGGAGGACTGGTTCAACCTGGCGGCTC | 759 |
| | | TCTGAGACTGTCTTGTGCCGCCAGCGGCTTCACCTTTAGCTTCTATTCTC | |
| | | TGAGCTGGGTCCGACAGGCCCCTGGAAAAGGACTTGAATGGGTGTCCGCC | |
| | | ATCAGCGGCGGAGGCGGAGGAAGCTATTACGCCGATAGCGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCATTTGATCTGTGGGGCCAGGGCACAATGGT | |
| | | CACAGTCTCGAGC | |
| | VL (DNA) | GCGAGCGTTCTGACCCAGCCTCCGAGCGTTAGCGCAGCACCGGGTCAGAA | 760 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGACTTGGACTGGTGCAGGTCGTATCTTTGTG | |
| | | TTCGGCGGTGGTACCAAGTTAACCGTGCTG | |
| D-109 | H-CDR1 | QTAMS | 761 |
| | H-CDR2 | AASYSGYSTYYADSVKG | 762 |
| | H-CDR3 | DSRDAYGVAFDL | 763 |
| | VH (aa) | EVQLLESGGGLVQPGGSLRLSCAASGFTFSQTAMSWVRQAPGKGLEWVSA | 764 |
| | | ASYSGYSTYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 765 |
| | L-CDR2 | DNNKRPS | 766 |
| | L-CDR3 | LTWTGAGRIFV | 767 |
| | VL (aa) | ASVLTQPPSVSAAPGQKVTISCSGSSSNIGNNYVSWYQQLPGTAPKLLIY | 768 |
| | | DNNKRPSGIPDRFSGSKSGTSATLGITGLQAEDEADYYCLTWTGAGRIFV | |
| | | FGGGTKLTVL | |
| | VH (DNA) | GAAGTGCAGCTGCTTGAATCTGGCGGAGGACTGGTTCAACCTGGCGGCTC | 769 |
| | | TCTGAGACTGTCTTGTGCCGCCAGCGGCTTCACCTTTAGCCAAACAGCCA | |
| | | TGAGCTGGGTCCGACAGGCCCCTGGAAAAGGACTTGAATGGGTGTCCGCC | |
| | | GCCAGCTACTCTGGCTACAGCACATATTACGCCGATAGCGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCATTTGATCTGTGGGGCCAGGGCACAATGGT | |
| | | CACAGTCTCGAGC | |
| | VL (DNA) | GCGAGCGTTCTGACCCAGCCTCCGAGCGTTAGCGCAGCACCGGGTCAGAA | 770 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGACTTGGACTGGTGCAGGTCGTATCTTTGTG | |
| | | TTCGGCGGTGGTACCAAGTTAACCGTGCTG | |
| D-110 | H-CDR1 | SSAVS | 771 |
| | H-CDR2 | AISYYGGAQYYADSVKG | 772 |
| | H-CDR3 | DSRDAYGVAFDL | 773 |
| | VH (aa) | EVQLLESGGGLVQPGGSLRLSCAASGFTFSSSAVSWVRQAPGKGLEWVSA | 774 |
| | | ISYYGGAQYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 775 |
| | L-CDR2 | DNNKRPS | 776 |
| | L-CDR3 | LTWTGAGRIFV | 777 |
| | VL (aa) | ASVLTQPPSVSAAPGQKVTISCSGSSSNIGNNYVSWYQQLPGTAPKLLIY | 778 |
| | | DNNKRPSGIPDRFSGSKSGTSATLGITGLQAEDEADYYCLTWTGAGRIFV | |
| | | FGGGTKLTVL | |
| | VH (DNA) | GAAGTGCAGCTGCTTGAATCTGGCGGAGGACTGGTTCAACCTGGCGGCTC | 779 |
| | | TCTGAGACTGTCTTGTGCCGCCAGCGGCTTCACCTTTAGCAGCTCTGCCG | |
| | | TTAGCTGGGTCCGACAGGCCCCTGGAAAAGGACTTGAATGGGTGTCCGCC | |
| | | ATCAGCTACTACGGCGGAGCACAGTATTACGCCGATAGCGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCATTTGATCTGTGGGGCCAGGGCACAATGGT | |
| | | CACAGTCTCGAGC | |
| | VL (DNA) | GCGAGCGTTCTGACCCAGCCTCCGAGCGTTAGCGCAGCACCGGGTCAGAA | 780 |
| | | AGTTACCATTAGCTGTAGCGGTAGCAGCAGCAATATTGGTAATAACTATG | |
| | | TTAGCTGGTATCAGCAGCTGCCTGGCACCGCACCGAAACTGCTGATTTAT | |
| | | GATAATAACAAACGTCCGAGCGGTATTCCGGATCGTTTTAGCGGTAGTAA | |
| | | AAGCGGCACCAGCGCAACCCTGGGTATTACCGGTCTGCAGGCAGAAGACG | |
| | | AGGCTGATTATTATTGCCTGACTTGGACTGGTGCAGGTCGTATCTTTGTG | |
| | | TTCGGCGGTGGTACCAAGTTAACCGTGCTG | |
| D-111 | H-CDR1 | SYAMS | 781 |
| | H-CDR2 | AISGSGGSTYYADSVKG | 782 |
| | H-CDR3 | DSRDAYGVAFDL | 783 |
| | VH (aa) | EVQLLESGGGLVQPGGSLRLSCAASGFTFSSYAMSWVRQAPGKGLEWVSA | 784 |
| | | ISGSGGSTYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 785 |
| | L-CDR2 | DNNKRPS | 786 |
| | L-CDR3 | LSYKLSPGAYV | 787 |
| | VL (aa) | ASVLTQPPSVSAAPGQKVTISCSGSSSNIGNNYVSWYQQLPGTAPKLLIY | 788 |
| | | DNNKRPSGIPDRFSGSKSGTSATLGITGLQAEDEADYYCLSYKLSPGAYV | |
| | | FGGGTKLTVL | |
| | VH (DNA) | GAAGTTCAGCTGCTGGAAAGCGGTGGTGGTCTGGTTCAGCCTGGTGGTAG | 789 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTATATTACTGTGCGAGAGACTCA | |
| | | AGAGATGCCTACGGGGTTGCTTTTGATCTCTGGGGCCAAGGGACAATGGT | |
| | | CACCGTCTCGAGC | |
| | VL (DNA) | GCCTCTGTGCTGACACAGCCTCCATCCGTTTCTGCTGCCCCTGGCCAGAA | 790 |
| | | AGTGACCATCAGCTGTAGCGGCAGCAGCAGCAACATCGGCAACAACTACG | |
| | | TGTCCTGGTATCAGCAGCTGCCCGGCACAGCTCCCAAACTGCTGATCTAC | |
| | | GACAACAACAAGCGGCCCAGCGGCATCCCCGATAGATTTTCTGGCAGCAA | |
| | | GAGCGGCACCAGCGCCACACTGGGAATTACAGGACTGCAGGCCGAGGACG | |
| | | AGGCCGACTACTACTGTCTGAGCTACAAGCTGAGCCCTGGCGCCTATGTG | |
| | | TTTGGCGGAGGTACCAAGCTGACAGTGCTG | |
| D-112 | H-CDR1 | SYAMS | 791 |
| | H-CDR2 | AISGSGGSTYYADSVKG | 792 |
| | H-CDR3 | DSRDAYGVAFDL | 793 |
| | VH (aa) | EVQLLESGGGLVQPGGSLRLSCAASGFTFSSYAMSWVRQAPGKGLEWVSA | 794 |
| | | ISGSGGSTYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 795 |
| | L-CDR2 | DNNKRPS | 796 |
| | L-CDR3 | QSYGHRSFV | 797 |
| | VL (aa) | ASVLTQPPSVSAAPGQKVTISCSGSSSNIGNNYVSWYQQLPGTAPKLLIY | 798 |
| | | DNNKRPSGIPDRFSGSKSGTSATLGITGLQAEDEADYYCQSYGHRSFVFG | |
| | | GGTKLTVL | |
| | VH (DNA) | GAAGTTCAGCTGCTGGAAAGCGGTGGTGGTCTGGTTCAGCCTGGTGGTAG | 799 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTATATTACTGTGCGAGAGACTCA | |
| | | AGAGATGCCTACGGGGTTGCTTTTGATCTCTGGGGCCAAGGGACAATGGT | |
| | | CACCGTCTCGAGC | |
| | VL (DNA) | GCCTCTGTGCTGACACAGCCTCCATCTGTTTCTGCTGCCCCTGGCCAGAA | 800 |
| | | AGTGACCATCAGCTGTAGCGGCAGCAGCAGCAACATCGGCAACAACTACG | |
| | | TGTCCTGGTATCAGCAGCTGCCCGGAACAGCCCCTAAACTGCTGATCTAC | |
| | | GACAACAACAAGCGGCCCAGCGGCATCCCTGATAGATTTTCTGGCAGCAA | |
| | | GAGCGGCACAAGCGCCACACTGGGAATTACAGGACTGCAGGCCGAGGACG | |
| | | AGGCCGATTACTACTGTCAGTCTTACGGCCACCGGTCCTTCGTGTTCGGC | |
| | | GGCGGTACCAAGCTGACAGTGCTG | |
| D-113 | H-CDR1 | SYAMS | 801 |
| | H-CDR2 | AISGSGGSTYYADSVKG | 802 |
| | H-CDR3 | DSRDAYGVAFDL | 803 |
| | VH (aa) | EVQLLESGGGLVQPGGSLRLSCAASGFTFSSYAMSWVRQAPGKGLEWVSA | 804 |
| | | ISGSGGSTYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 805 |
| | L-CDR2 | DNNKRPS | 806 |
| | L-CDR3 | QSYESRLFV | 807 |
| | VL (aa) | ASVLTQPPSVSAAPGQKVTISCSGSSSNIGNNYVSWYQQLPGTAPKLLIY | 808 |
| | | DNNKRPSGIPDRFSGSKSGTSATLGITGLQAEDEADYYCQSYESRLFVFG | |
| | | GGTKLTVL | |
| | VH (DNA) | GAAGTTCAGCTGCTGGAAAGCGGTGGTGGTCTGGTTCAGCCTGGTGGTAG | 809 |
| | | CCTGCGTCTGAGCTGTGCAGCAAGCGGTTTTACCTTTAGCAGCTATGCAA | |
| | | TGAGCTGGGTTCGTCAGGCACCTGGTAAAGGTCTGGAATGGGTTAGCGCA | |
| | | ATTAGCGGTAGCGGTGGTAGCACCTATTATGCAGATAGCGTTAAAGGTCG | |
| | | CTTTACCATTAGCCGTGATAATAGCAAAAATACCCTGTACCTGCAGATGA | |
| | | ATAGTCTGCGTGCAGAAGATACGGCCGTATATTACTGTGCGAGAGACTCA | |
| | | AGAGATGCCTACGGGGTTGCTTTTGATCTCTGGGGCCAAGGGACAATGGT | |
| | | CACCGTCTCGAGC | |
| | VL (DNA) | GCCTCTGTGCTGACACAGCCTCCATCTGTTTCTGCTGCCCCTGGCCAGAA | 810 |
| | | AGTGACCATCAGCTGTAGCGGCAGCAGCAGCAACATCGGCAACAACTACG | |
| | | TGTCCTGGTATCAGCAGCTGCCCGGAACAGCCCCTAAACTGCTGATCTAC | |
| | | GACAACAACAAGCGGCCCAGCGGCATCCCTGATAGATTTTCTGGCAGCAA | |
| | | GAGCGGCACAAGCGCCACACTGGGAATTACAGGACTGCAGGCCGAGGACG | |
| | | AGGCCGATTACTACTGTCAGAGCTACGAGAGCCGGCTGTTCGTGTTCGGC | |
| | | GGCGGTACCAAGCTGACAGTGCTG | |
| D-114 | H-CDR1 | FSALS | 811 |
| | H-CDR2 | AISYGGGSKYYADSVKG | 812 |
| | H-CDR3 | DSRDAYGVAFDL | 813 |
| | VH (aa) | EVQLLESGGGLVQPGGSLRLSCAASGFTFQFSALSWVRQAPGKGLEWVSA | 814 |
| | | ISYGGGSKYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 815 |
| | L-CDR2 | DNNKRPS | 816 |
| | L-CDR3 | LSYKLSPGAYV | 817 |
| | VL (aa) | ASVLTQPPSVSAAPGQKVTISCSGSSSNIGNNYVSWYQQLPGTAPKLLIY | 818 |
| | | DNNKRPSGIPDRFSGSKSGTSATLGITGLQAEDEADYYCLSYKLSPGAYV | |
| | | FGGGTKLTVL | |
| | VH (DNA) | GAAGTGCAGCTGCTTGAATCTGGCGGAGGACTGGTTCAGCCTGGCGGATC | 819 |
| | | TCTGAGACTGTCTTGTGCCGCCAGCGGCTTCACCTTCCAGTTTTCTGCCC | |
| | | TGAGCTGGGTCCGACAGGCCCCTGGAAAAGGACTGGAATGGGTGTCCGCC | |
| | | ATCTCTTACGGCGGAGGCAGCAAGTACTACGCCGACTCTGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCCTTTGATCTGTGGGGCCAGGGCACAATGGT | |
| | | CACAGTCTCGAGC | |
| | VL (DNA) | GCCTCTGTGCTGACACAGCCTCCATCCGTTTCTGCTGCCCCTGGCCAGAA | 820 |
| | | AGTGACCATCAGCTGTAGCGGCAGCAGCAGCAACATCGGCAACAACTACG | |
| | | TGTCCTGGTATCAGCAGCTGCCCGGCACAGCTCCCAAACTGCTGATCTAC | |
| | | GACAACAACAAGCGGCCCAGCGGCATCCCCGATAGATTTTCTGGCAGCAA | |
| | | GAGCGGCACCAGCGCCACACTGGGAATTACAGGACTGCAGGCCGAGGACG | |
| | | AGGCCGACTACTACTGTCTGAGCTACAAGCTGAGCCCTGGCGCCTATGTG | |
| | | TTTGGCGGAGGTACCAAGCTGACAGTGCTG | |
| D-115 | H-CDR1 | FSALS | 821 |
| | H-CDR2 | AISYGGGSKYYADSVKG | 822 |
| | H-CDR3 | DSRDAYGVAFDL | 823 |
| | VH (aa) | EVQLLESGGGLVQPGGSLRLSCAASGFTFQFSALSWVRQAPGKGLEWVSA | 824 |
| | | ISYGGGSKYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 825 |
| | L-CDR2 | DNNKRPS | 826 |
| | L-CDR3 | QSYESRLFV | 827 |
| | VL (aa) | ASVLTQPPSVSAAPGQKVTISCSGSSSNIGNNYVSWYQQLPGTAPKLLIY | 828 |
| | | DNNKRPSGIPDRFSGSKSGTSATLGITGLQAEDEADYYCQSYESRLFVFG | |
| | | GGTKLTVL | |
| | VH (DNA) | GAAGTGCAGCTGCTTGAATCTGGCGGAGGACTGGTTCAGCCTGGCGGATC | 829 |
| | | TCTGAGACTGTCTTGTGCCGCCAGCGGCTTCACCTTCCAGTTTTCTGCCC | |
| | | TGAGCTGGGTCCGACAGGCCCCTGGAAAAGGACTGGAATGGGTGTCCGCC | |
| | | ATCTCTTACGGCGGAGGCAGCAAGTACTACGCCGACTCTGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCCTTTGATCTGTGGGGCCAGGGCACAATGGT | |
| | | CACAGTCTCGAGC | |
| | VL (DNA) | GCCTCTGTGCTGACACAGCCTCCATCTGTTTCTGCTGCCCCTGGCCAGAA | 830 |
| | | AGTGACCATCAGCTGTAGCGGCAGCAGCAGCAACATCGGCAACAACTACG | |
| | | TGTCCTGGTATCAGCAGCTGCCCGGAACAGCCCCTAAACTGCTGATCTAC | |
| | | GACAACAACAAGCGGCCCAGCGGCATCCCTGATAGATTTTCTGGCAGCAA | |
| | | GAGCGGCACAAGCGCCACACTGGGAATTACAGGACTGCAGGCCGAGGACG | |
| | | AGGCCGATTACTACTGTCAGAGCTACGAGAGCCGGCTGTTCGTGTTCGGC | |
| | | GGCGGTACCAAGCTGACAGTGCTG | |
| D-116 | H-CDR1 | HSAIS | 831 |
| | H-CDR2 | AISYGGGSQYYADSVKG | 832 |
| | H-CDR3 | DSRDAYGVAFDL | 833 |
| | VH (aa) | EVQLLESGGGLVQPGGSLRLSCAASGFTFKHSAISWVRQAPGKGLEWVSA | 834 |
| | | ISYGGGSQYYADSVKGRFTISRDNSKNTLYLQMNSLRAEDTAVYYCARDS | |
| | | RDAYGVAFDLWGQGTMVTVSS | |
| | L-CDR1 | SGSSSNIGNNYVS | 835 |
| | L-CDR2 | DNNKRPS | 836 |
| | L-CDR3 | QSYGHRSFV | 837 |
| | VL (aa) | ASVLTQPPSVSAAPGQKVTISCSGSSSNIGNNYVSWYQQLPGTAPKLLIY | 838 |
| | | DNNKRPSGIPDRFSGSKSGTSATLGITGLQAEDEADYYCQSYGHRSFVFG | |
| | | GGTKLTVL | |
| | VH (DNA) | GAAGTGCAGCTGCTTGAATCTGGCGGAGGACTGGTTCAACCTGGCGGCTC | 839 |
| | | TCTGAGACTGTCTTGTGCCGCCAGCGGCTTCACCTTTAAGCACAGCGCCA | |
| | | TTAGCTGGGTCCGACAGGCCCCTGGAAAAGGACTTGAATGGGTGTCCGCC | |
| | | ATCAGCTATGGCGGCGGAAGCCAATATTACGCCGATAGCGTGAAGGGCAG | |
| | | ATTCACCATCAGCCGGGACAACAGCAAGAACACCCTGTACCTGCAGATGA | |
| | | ACAGCCTGAGAGCCGAGGACACCGCCGTGTACTACTGTGCCAGAGACAGC | |
| | | AGAGATGCCTACGGCGTGGCATTTGATCTGTGGGGCCAGGGCACAATGGT | |
| | | CACAGTCTCGAGC | |
| | VL (DNA) | GCCTCTGTGCTGACACAGCCTCCATCTGTTTCTGCTGCCCCTGGCCAGAA | 840 |
| | | AGTGACCATCAGCTGTAGCGGCAGCAGCAGCAACATCGGCAACAACTACG | |
| | | TGTCCTGGTATCAGCAGCTGCCCGGAACAGCCCCTAAACTGCTGATCTAC | |
| | | GACAACAACAAGCGGCCCAGCGGCATCCCTGATAGATTTTCTGGCAGCAA | |
| | | GAGCGGCACAAGCGCCACACTGGGAATTACAGGACTGCAGGCCGAGGACG | |
| | | AGGCCGATTACTACTGTCAGTCTTACGGCCACCGGTCCTTCGTGTTCGGC | |
| | | GGCGGTACCAAGCTGACAGTGCTG | |
| D-201 | H-CDR1 | HAWIS | 841 |
| | H-CDR2 | QIKGGPGSGGTSYAEPVKG | 842 |
| | H-CDR3 | LGIYSGFDY | 843 |
| | VH (aa) | EVQLVESGGGLVKPGGSLRLSCAASGFTFSHAWISWVRQAPGKGLEWVGQ | 844 |
| | | IKGGPGSGGTSYAEPVKGRFTISRDDSKNTLYLQMNSLKTEDTAVYYCAR | |
| | | LGIYSGFDYWGQGTLVTVSS | |
| | L-CDR1 | SGDKLGDKYAS | 845 |
| | L-CDR2 | QDSKRPS | 846 |
| | L-CDR3 | HSYTGKPSQVV | 847 |
| | VL (aa) | SYELTQPPSVSVSPGQTASITCSGDKLGDKYASWYQQKPGQSPVLVIYQD | 848 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTGGTTAAGCCTGGCGGATC | 849 |
| | | TCTGAGACTGAGCTGTGCCGCCAGCGGCTTCACATTTTCTCACGCCTGGA | |
| | | TCAGCTGGGTCCGACAGGCTCCTGGAAAAGGCCTGGAATGGGTCGGACAG | |
| | | ATCAAAGGCGGACCTGGCTCTGGCGGAACAAGCTATGCCGAGCCTGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGTGCCAGA | |
| | | CTGGGCATCTACTCCGGCTTCGATTATTGGGGCCAGGGCACCCTGGTTAC | |
| | | AGTCTCGAGC | |
| | VL (DNA) | AGCTATGAACTGACCCAGCCTCCGAGCGTTAGCGTTAGTCCGGGTCAGAC | 850 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-202 | H-CDR1 | YAWIS | 851 |
| | H-CDR2 | QIKSGSDASQTSYAAPVKG | 852 |
| | H-CDR3 | LGIYSGFDY | 853 |
| | VH (aa) | EVQLVESGGGLVKPGGSLRLSCAASGFTFTYAWISWVRQAPGKGLEWVGQ | 854 |
| | | IKSGSDASQTSYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 855 |
| | L-CDR2 | QDSKRPS | 856 |
| | L-CDR3 | HSYTGKPSQVV | 857 |
| | VL (aa) | SYELTQPPSVSVSPGQTASITCSGDKLGDKYASWYQQKPGQSPVLVIYQD | 858 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTTGTGAAACCTGGCGGCTC | 859 |
| | | TCTGAGACTGTCTTGTGCCGCCAGCGGCTTCACCTTCACATACGCCTGGA | |
| | | TTAGCTGGGTTCGACAGGCCCCTGGAAAAGGCCTGGAATGGGTCGGACAG | |
| | | ATCAAGAGCGGAAGCGACGCCAGCCAGACATCTTACGCTGCTCCAGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGCGCCAGA | |
| | | CTGGGCATCTACAGCGGCTTCGACTACTGGGGCCAGGGCACCCTGGTGAC | |
| | | CGTCTCGAGC | |
| | VL (DNA) | AGCTATGAACTGACCCAGCCTCCGAGCGTTAGCGTTAGTCCGGGTCAGAC | 860 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-203 | H-CDR1 | SLWMS | 861 |
| | H-CDR2 | QIKSSTSGSGTSYGAPVKG | 862 |
| | H-CDR3 | LGIYSGFDY | 863 |
| | VH (aa) | EVQLVESGGGLVKPGGSLRLSCAASGFTFSSLWMSWVRQAPGKGLEWVGQ | 864 |
| | | IKSSTSGSGTSYGAPVKGRFTISRDDSKNTLYLQMNSLKTEDTAVYYCAR | |
| | | LGIYSGFDYWGQGTLVTVSS | |
| | L-CDR1 | SGDKLGDKYAS | 865 |
| | L-CDR2 | QDSKRPS | 866 |
| | L-CDR3 | HSYTGKPSQVV | 867 |
| | VL (aa) | SYELTQPPSVSVSPGQTASITCSGDKLGDKYASWYQQKPGQSPVLVIYQD | 868 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTTGTGAAACCTGGCGGCTC | 869 |
| | | TCTGAGACTGTCTTGTGCCGCCAGCGGCTTCACCTTCAGCAGTCTTTGGA | |
| | | TGAGCTGGGTTCGACAGGCCCCTGGAAAAGGCCTGGAATGGGTCGGACAA | |
| | | ATCAAGAGCAGTACCAGCGGCTCTGGCACCTCTTACGGTGCTCCAGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGCGCCAGA | |
| | | CTGGGCATCTACAGCGGCTTCGACTACTGGGGCCAGGGCACCCTGGTGAC | |
| | | CGTCTCGAGC | |
| | VL (DNA) | AGCTATGAACTGACCCAGCCTCCGAGCGTTAGCGTTAGTCCGGGTCAGAC | 870 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-204 | H-CDR1 | SAWIS | 871 |
| | H-CDR2 | QIKSKSEASSTTYAAPVKG | 872 |
| | H-CDR3 | LGIYSGFDY | 873 |
| | VH (aa) | EVQLVESGGGLVKPGGSLRLSCAASGFTFESAWISWVRQAPGKGLEWVGQ | 874 |
| | | IKSKSEASSTTYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 875 |
| | L-CDR2 | QDSKRPS | 876 |
| | L-CDR3 | HSYTGKPSQVV | 877 |
| | VL (aa) | SYELTQPPSVSVSPGQTASITCSGDKLGDKYASWYQQKPGQSPVLVIYQD | 878 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTTGTGAAACCTGGCGGCTC | 879 |
| | | TCTGAGACTGTCTTGTGCCGCCAGCGGCTTCACCTTCGAAAGCGCCTGGA | |
| | | TTAGCTGGGTTCGACAGGCCCCTGGAAAAGGCCTGGAATGGGTCGGACAG | |
| | | ATCAAGAGCAAGAGCGAGGCCAGCAGCACCACATACGCTGCTCCAGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGCGCCAGA | |
| | | CTGGGCATCTACAGCGGCTTCGACTACTGGGGCCAGGGCACCCTGGTGAC | |
| | | CGTCTCGAGC | |
| | VL (DNA) | AGCTATGAACTGACCCAGCCTCCGAGCGTTAGCGTTAGTCCGGGTCAGAC | 880 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-205 | H-CDR1 | HAWMS | 881 |
| | H-CDR2 | QIKSKSDASKTTYAAPVKG | 882 |
| | H-CDR3 | LGIYSGFDY | 883 |
| | VH (aa) | EVQLVESGGGLVKPGGSLRLSCAASGFTFSHAWMSWVRQAPGKGLEWVGQ | 884 |
| | | IKSKSDASKTTYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 885 |
| | L-CDR2 | QDSKRPS | 886 |
| | L-CDR3 | HSYTGKPSQVV | 887 |
| | VL (aa) | SYELTQPPSVSVSPGQTASITCSGDKLGDKYASWYQQKPGQSPVLVIYQD | 888 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTTGTGAAACCTGGCGGCTC | 889 |
| | | TCTGAGACTGTCTTGTGCCGCCAGCGGCTTCACCTTCAGCCATGCCTGGA | |
| | | TGAGCTGGGTTCGACAGGCCCCTGGAAAAGGCCTGGAATGGGTCGGACAG | |
| | | ATCAAGAGCAAGAGCGACGCCAGCAAGACCACATACGCTGCTCCAGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGCGCCAGA | |
| | | CTGGGCATCTACAGCGGCTTCGACTACTGGGGCCAGGGCACCCTGGTGAC | |
| | | CGTCTCGAGC | |
| | VL (DNA) | AGCTATGAACTGACCCAGCCTCCGAGCGTTAGCGTTAGTCCGGGTCAGAC | 890 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-206 | H-CDR1 | YTWIS | 891 |
| | H-CDR2 | QIKSTTSASSIDYASPVKG | 892 |
| | H-CDR3 | LGIYSGFDY | 893 |
| | VH (aa) | EVQLVESGGGLVKPGGSLRLSCAASGFTFSYTWISWVRQAPGKGLEWVGQ | 894 |
| | | IKSTTSASSIDYASPVKGRFTISRDDSKNTLYLQMNSLKTEDTAVYYCAR | |
| | | LGIYSGFDYWGQGTLVTVSS | |
| | L-CDR1 | SGDKLGDKYAS | 895 |
| | L-CDR2 | QDSKRPS | 896 |
| | L-CDR3 | HSYTGKPSQVV | 897 |
| | VL (aa) | SYELTQPPSVSVSPGQTASITCSGDKLGDKYASWYQQKPGQSPVLVIYQD | 898 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTTGTGAAACCTGGCGGCTC | 899 |
| | | TCTGAGACTGTCTTGTGCCGCCAGCGGCTTCACCTTCAGCTACACATGGA | |
| | | TCAGCTGGGTTCGACAGGCCCCTGGAAAAGGCCTGGAATGGGTCGGACAA | |
| | | ATCAAGAGCACCACCAGCGCCAGCAGCATCGATTACGCTTCTCCAGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGCGCCAGA | |
| | | CTGGGCATCTACAGCGGCTTCGACTACTGGGGCCAGGGCACCCTGGTGAC | |
| | | CGTCTCGAGC | |
| | VL (DNA) | AGCTATGAACTGACCCAGCCTCCGAGCGTTAGCGTTAGTCCGGGTCAGAC | 900 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-207 | H-CDR1 | YAYMY | 901 |
| | H-CDR2 | HIKSSTDGSGKEYSAPVKG | 902 |
| | H-CDR3 | LGIYSGFDY | 903 |
| | VH (aa) | EVQLVESGGGLVKPGGSLRLSCAASGFTFTYAYMYWVRQAPGKGLEWVGH | 904 |
| | | IKSSTDGSGKEYSAPVKGRFTISRDDSKNTLYLQMNSLKTEDTAVYYCAR | |
| | | LGIYSGFDYWGQGTLVTVSS | |
| | L-CDR1 | SGDKLGDKYAS | 905 |
| | L-CDR2 | QDSKRPS | 906 |
| | L-CDR3 | HSYTGKPSQVV | 907 |
| | VL (aa) | SYELTQPPSVSVSPGQTASITCSGDKLGDKYASWYQQKPGQSPVLVIYQD | 908 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTTGTGAAACCTGGCGGCTC | 909 |
| | | TCTGAGACTGTCTTGTGCCGCCAGCGGCTTCACCTTCACCTACGCCTACA | |
| | | TGTACTGGGTTCGACAGGCCCCTGGAAAAGGCCTGGAATGGGTCGGACAT | |
| | | ATCAAGAGCAGTACCGACGGCAGCGGCAAAGAATACTCTGCTCCAGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGCGCCAGA | |
| | | CTGGGCATCTACAGCGGCTTCGACTACTGGGGCCAGGGCACCCTGGTGAC | |
| | | CGTCTCGAGC | |
| | VL (DNA) | AGCTATGAACTGACCCAGCCTCCGAGCGTTAGCGTTAGTCCGGGTCAGAC | 910 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-208 | H-CDR1 | YAWIS | 911 |
| | H-CDR2 | QIKSSSDASSTTYAAPVKG | 912 |
| | H-CDR3 | LGIYSGFDY | 913 |
| | VH (aa) | EVQLVESGGGLVKPGGSLRLSCAASGFTFSYAWISWVRQAPGKGLEWVGQ | 914 |
| | | IKSSSDASSTTYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 915 |
| | L-CDR2 | QDSKRPS | 916 |
| | L-CDR3 | HSYTGKPSQVV | 917 |
| | VL (aa) | SYELTQPPSVSVSPGQTASITCSGDKLGDKYASWYQQKPGQSPVLVIYQD | 918 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTTGTGAAACCTGGCGGCTC | 919 |
| | | TCTGAGACTGTCTTGTGCCGCCAGCGGCTTCACCTTCAGCTATGCCTGGA | |
| | | TTAGCTGGGTTCGACAGGCCCCTGGAAAAGGCCTGGAATGGGTCGGACAG | |
| | | ATCAAGAGCAGCAGCGACGCCAGCTCTACCACATACGCTGCTCCAGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGCGCCAGA | |
| | | CTGGGCATCTACAGCGGCTTCGACTACTGGGGCCAGGGCACCCTGGTGAC | |
| | | CGTCTCGAGC | |
| | VL (DNA) | AGCTATGAACTGACCCAGCCTCCGAGCGTTAGCGTTAGTCCGGGTCAGAC | 920 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-209 | H-CDR1 | HAWIT | 921 |
| | H-CDR2 | QIKSSSDASETSYAAPVKG | 922 |
| | H-CDR3 | LGIYSGFDY | 923 |
| | VH (aa) | EVQLVESGGGLVKPGGSLRLSCAASGFTFSHAWITWVRQAPGKGLEWVGQ | 924 |
| | | IKSSSDASETSYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 925 |
| | L-CDR2 | QDSKRPS | 926 |
| | L-CDR3 | HSYTGKPSQVV | 927 |
| | VL (aa) | SYELTQPPSVSVSPGQTASITCSGDKLGDKYASWYQQKPGQSPVLVIYQD | 928 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTTGTGAAACCTGGCGGCTC | 929 |
| | | TCTGAGACTGTCTTGTGCCGCCAGCGGCTTCACCTTCAGCCATGCCTGGA | |
| | | TTACATGGGTTCGACAGGCCCCTGGAAAAGGCCTGGAATGGGTCGGACAG | |
| | | ATCAAGAGCAGCAGCGACGCCAGCGAGACATCTTACGCTGCTCCAGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGCGCCAGA | |
| | | CTGGGCATCTACAGCGGCTTCGACTACTGGGGCCAGGGCACCCTGGTGAC | |
| | | CGTCTCGAGC | |
| | VL (DNA) | AGCTATGAACTGACCCAGCCTCCGAGCGTTAGCGTTAGTCCGGGTCAGAC | 930 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-210 | H-CDR1 | SAWVS | 931 |
| | H-CDR2 | QIKGYTSGGTITYAAPVKG | 932 |
| | H-CDR3 | LGIYSGFDY | 933 |
| | VH (aa) | EVQLVESGGGLVKPGGSLRLSCAASGFTFSSAWVSWVRQAPGKGLEWVGQ | 934 |
| | | IKGYTSGGTITYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 935 |
| | L-CDR2 | QDSKRPS | 936 |
| | L-CDR3 | HSYTGKPSQVV | 937 |
| | VL (aa) | SYELTQPPSVSVSPGQTASITCSGDKLGDKYASWYQQKPGQSPVLVIYQD | 938 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTTGTGAAACCTGGCGGCTC | 939 |
| | | TCTGAGACTGTCTTGTGCCGCCAGCGGCTTCACCTTCAGCTCTGCCTGGG | |
| | | TTAGCTGGGTTCGACAGGCCCCTGGAAAAGGCCTGGAATGGGTCGGACAG | |
| | | ATCAAGGGCTATACCAGCGGCGGCACCATCACATACGCTGCTCCAGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGCGCCAGA | |
| | | CTGGGCATCTACAGCGGCTTCGACTACTGGGGCCAGGGCACCCTGGTGAC | |
| | | CGTCTCGAGC | |
| | VL (DNA) | AGCTATGAACTGACCCAGCCTCCGAGCGTTAGCGTTAGTCCGGGTCAGAC | 940 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-211 | H-CDR1 | HAWIS | 941 |
| | H-CDR2 | RIKGSTEASQTDYAAPVKG | 942 |
| | H-CDR3 | LGIYSGFDY | 943 |
| | VH (aa) | EVQLVESGGGLVKPGGSLRLSCAASGFTFQHAWISWVRQAPGKGLEWVGR | 944 |
| | | IKGSTEASQTDYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 945 |
| | L-CDR2 | QDSKRPS | 946 |
| | L-CDR3 | HSYTGKPSQVV | 947 |
| | VL (aa) | SYELTQPPSVSVSPGQTASITCSGDKLGDKYASWYQQKPGQSPVLVIYQD | 948 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTTGTGAAACCTGGCGGCTC | 949 |
| | | TCTGAGACTGTCTTGTGCCGCCAGCGGCTTCACCTTCCAACATGCCTGGA | |
| | | TTAGCTGGGTTCGACAGGCCCCTGGAAAAGGCCTGGAATGGGTCGGAAGA | |
| | | ATCAAGGGCAGCACCGAGGCCAGCCAGACAGATTACGCTGCTCCAGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGCGCCAGA | |
| | | CTGGGCATCTACAGCGGCTTCGACTACTGGGGCCAGGGCACCCTGGTGAC | |
| | | CGTCTCGAGC | |
| | VL (DNA) | AGCTATGAACTGACCCAGCCTCCGAGCGTTAGCGTTAGTCCGGGTCAGAC | 950 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-212 | H-CDR1 | SLWIS | 951 |
| | H-CDR2 | QIKGKTESSSTTYEAPVKG | 952 |
| | H-CDR3 | LGIYSGFDY | 953 |
| | VH (aa) | EVQLVESGGGLVKPGGSLRLSCAASGFTFSSLWISWVRQAPGKGLEWVGQ | 954 |
| | | IKGKTESSSTTYEAPVKGRFTISRDDSKNTLYLQMNSLKTEDTAVYYCAR | |
| | | LGIYSGFDYWGQGTLVTVSS | |
| | L-CDR1 | SGDKLGDKYAS | 955 |
| | L-CDR2 | QDSKRPS | 956 |
| | L-CDR3 | HSYTGKPSQVV | 957 |
| | VL (aa) | SYELTQPPSVSVSPGQTASITCSGDKLGDKYASWYQQKPGQSPVLVIYQD | 958 |
| | | SKRPSGIPERFSGSNSGNTATLTISGTQAEDEADYYCHSYTGKPSQVVFG | |
| | | GGTKLTVL | |
| | VH (DNA) | GAAGTGCAGCTGGTTGAATCTGGCGGCGGACTTGTGAAACCTGGCGGCTC | 959 |
| | | TCTGAGACTGTCTTGTGCCGCCAGCGGCTTCACCTTCAGCAGTCTGTGGA | |
| | | TTAGCTGGGTTCGACAGGCCCCTGGAAAAGGCCTGGAATGGGTCGGACAG | |
| | | ATCAAGGGCAAGACCGAGAGCAGCAGCACCACATACGAAGCTCCAGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGCGCCAGA | |
| | | CTGGGCATCTACAGCGGCTTCGACTACTGGGGCCAGGGCACCCTGGTGAC | |
| | | CGTCTCGAGC | |
| | VL (DNA) | AGCTATGAACTGACCCAGCCTCCGAGCGTTAGCGTTAGTCCGGGTCAGAC | 960 |
| | | CGCAAGCATTACCTGTAGCGGTGATAAACTGGGTGATAAATATGCAAGCT | |
| | | GGTATCAGCAGAAACCGGGTCAGTCACCGGTTCTGGTTATTTATCAGGAT | |
| | | AGCAAACGTCCGAGCGGTATTCCGGAACGTTTTAGCGGATCCAATAGTGG | |
| | | TAATACCGCAACACTGACCATTAGCGGCACCCAGGCTGAAGACGAGGCTG | |
| | | ATTATTATTGCCATTCTTACACTGGTAAACCATCTCAGGTTGTGTTCGGC | |
| | | GGTGGTACCAAGTTAACCGTGCTG | |
| D-213 | H-CDR1 | NAWMS | 961 |
| | H-CDR2 | RIKSKTDGGTTDYAAPVKG | 962 |
| | H-CDR3 | LGIYSGFDY | 963 |
| | VH (aa) | EVQLVESGGGLVKPGGSLRLSCAASGFTFSNAWMSWVRQAPGKGLEWVGR | 964 |
| | | IKSKTDGGTTDYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 965 |
| | L-CDR2 | QDSKRPS | 966 |
| | L-CDR3 | HTYSHRPEIVV | 967 |
| | VL (aa) | SYELTQPPSVSVSPGQTASITCSGDKLGDKYASWYQQKPGQSPVLVIYQD | 968 |
| | | SKRPSGIPERFSGSNSGNTATLTISGTQAEDEADYYCHTYSHRPEIVVFG | |
| | | GGTKLTVL | |
| | VH (DNA) | GAAGTTCAGCTGGTTGAAAGCGGTGGTGGTCTGGTTAAACCTGGTGGTAG | 969 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTATCTACTCTGGTTTTGATTACTGGGGCCAGGGCACCCTGGTTAC | |
| | | TGTCTCGAGC | |
| | VL (DNA) | AGCTACGAGCTGACACAGCCTCCAAGCGTGTCCGTGTCTCCTGGACAGAC | 970 |
| | | AGCCAGCATCACCTGTAGCGGCGATAAGCTGGGCGATAAGTACGCCAGCT | |
| | | GGTATCAGCAGAAGCCCGGCCAGTCTCCTGTGCTGGTCATCTACCAGGAC | |
| | | AGCAAGAGGCCTAGCGGCATCCCCGAGAGATTCAGCGGCAGCAATAGCGG | |
| | | CAATACCGCCACACTGACCATCAGCGGAACACAGGCCGAGGACGAGGCCG | |
| | | ATTACTACTGCCACACCTACAGCCACCGGCCTGAGATCGTGGTTTTTGGC | |
| | | GGAGGTACCAAGCTGACAGTGCTG | |
| D-214 | H-CDR1 | NAWMS | 971 |
| | H-CDR2 | RIKSKTDGGTTDYAAPVKG | 972 |
| | H-CDR3 | LGIYSGFDY | 973 |
| | VH (aa) | EVQLVESGGGLVKPGGSLRLSCAASGFTFSNAWMSWVRQAPGKGLEWVGR | 974 |
| | | IKSKTDGGTTDYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 975 |
| | L-CDR2 | QDSKRPS | 976 |
| | L-CDR3 | HSYLHRPSVTV | 977 |
| | VL (aa) | SYELTQPPSVSVSPGQTASITCSGDKLGDKYASWYQQKPGQSPVLVIYQD | 978 |
| | | SKRPSGIPERFSGSNSGNTATLTISGTQAEDEADYYCHSYLHRPSVTVFG | |
| | | GGTKLTVL | |
| | VH (DNA) | GAAGTTCAGCTGGTTGAAAGCGGTGGTGGTCTGGTTAAACCTGGTGGTAG | 979 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTATCTACTCTGGTTTTGATTACTGGGGCCAGGGCACCCTGGTTAC | |
| | | TGTCTCGAGC | |
| | VL (DNA) | AGCTACGAGCTGACACAGCCTCCTAGCGTTTCCGTGTCTCCTGGCCAGAC | 980 |
| | | AGCCAGCATCACATGTTCTGGCGACAAGCTGGGCGATAAGTACGCCAGCT | |
| | | GGTATCAGCAGAAGCCCGGACAGTCTCCCGTGCTGGTCATCTACCAGGAT | |
| | | AGCAAGAGGCCTAGCGGCATCCCTGAGAGATTCAGCGGCAGCAATAGCGG | |
| | | CAATACCGCCACACTGACAATCAGCGGAACACAGGCCGAGGACGAGGCCG | |
| | | ATTACTACTGTCACAGCTACCTGCACAGACCCAGCGTGACAGTGTTCGGC | |
| | | GGCGGTACCAAGCTGACAGTGCTG | |
| D-215 | H-CDR1 | NAWMS | 981 |
| | H-CDR2 | RIKSKTDGGTTDYAAPVKG | 982 |
| | H-CDR3 | LGIYSGFDY | 983 |
| | VH (aa) | EVQLVESGGGLVKPGGSLRLSCAASGFTFSNAWMSWVRQAPGKGLEWVGR | 984 |
| | | IKSKTDGGTTDYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 985 |
| | L-CDR2 | QDSKRPS | 986 |
| | L-CDR3 | HTYLHLPSLVV | 987 |
| | VL (aa) | SYELTQPPSVSVSPGQTASITCSGDKLGDKYASWYQQKPGQSPVLVIYQD | 988 |
| | | SKRPSGIPERFSGSNSGNTATLTISGTQAEDEADYYCHTYLHLPSLVVFG | |
| | | GGTKLTVL | |
| | VH (DNA) | GAAGTTCAGCTGGTTGAAAGCGGTGGTGGTCTGGTTAAACCTGGTGGTAG | 989 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTATCTACTCTGGTTTTGATTACTGGGGCCAGGGCACCCTGGTTAC | |
| | | TGTCTCGAGC | |
| | VL (DNA) | AGCTACGAGCTGACACAGCCTCCTAGCGTTTCCGTGTCTCCTGGCCAGAC | 990 |
| | | AGCCAGCATCACATGTTCTGGCGACAAGCTGGGCGATAAGTACGCCAGCT | |
| | | GGTATCAGCAGAAGCCCGGACAGTCTCCCGTGCTGGTCATCTACCAGGAT | |
| | | AGCAAGAGGCCTAGCGGCATCCCTGAGAGATTCAGCGGCAGCAATAGCGG | |
| | | CAATACCGCCACACTGACAATCAGCGGAACACAGGCCGAGGACGAGGCCG | |
| | | ATTACTACTGTCACACATACCTGCATCTGCCCAGCCTTGTGGTGTTCGGC | |
| | | GGCGGTACCAAGCTGACAGTGCTG | |
| D-216 | H-CDR1 | NAWMS | 991 |
| | H-CDR2 | RIKSKTDGGTTDYAAPVKG | 992 |
| | H-CDR3 | LGIYSGFDY | 993 |
| | VH (aa) | EVQLVESGGGLVKPGGSLRLSCAASGFTFSNAWMSWVRQAPGKGLEWVGR | 994 |
| | | IKSKTDGGTTDYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 995 |
| | L-CDR2 | QDSKRPS | 996 |
| | L-CDR3 | HSYLHRPETVV | 997 |
| | VL (aa) | SYELTQPPSVSVSPGQTASITCSGDKLGDKYASWYQQKPGQSPVLVIYQD | 998 |
| | | SKRPSGIPERFSGSNSGNTATLTISGTQAEDEADYYCHSYLHRPETVVFG | |
| | | GGTKLTVL | |
| | VH (DNA) | GAAGTTCAGCTGGTTGAAAGCGGTGGTGGTCTGGTTAAACCTGGTGGTAG | 999 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTATCTACTCTGGTTTTGATTACTGGGGCCAGGGCACCCTGGTTAC | |
| | | TGTCTCGAGC | |
| | VL (DNA) | AGCTACGAGCTGACACAGCCTCCTAGCGTTTCCGTGTCTCCTGGCCAGAC | 1000 |
| | | AGCCAGCATCACATGTTCTGGCGACAAGCTGGGCGATAAGTACGCCAGCT | |
| | | GGTATCAGCAGAAGCCCGGACAGTCTCCCGTGCTGGTCATCTACCAGGAT | |
| | | AGCAAGAGGCCTAGCGGCATCCCTGAGAGATTCAGCGGCAGCAATAGCGG | |
| | | CAATACCGCCACACTGACAATCAGCGGAACACAGGCCGAGGACGAGGCCG | |
| | | ATTACTACTGTCACAGCTACCTGCACAGACCCGAGACAGTGGTGTTCGGC | |
| | | GGCGGTACCAAGCTGACAGTGCTG | |
| D-217 | H-CDR1 | NAWMS | 1001 |
| | H-CDR2 | RIKSKTDGGTTDYAAPVKG | 1002 |
| | H-CDR3 | LGIYSGFDY | 1003 |
| | VH (aa) | EVQLVESGGGLVKPGGSLRLSCAASGFTFSNAWMSWVRQAPGKGLEWVGR | 1004 |
| | | IKSKTDGGTTDYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 1005 |
| | L-CDR2 | QDSKRPS | 1006 |
| | L-CDR3 | HSYSHRPEVVV | 1007 |
| | VL (aa) | SYELTQPPSVSVSPGQTASITCSGDKLGDKYASWYQQKPGQSPVLVIYQD | 1008 |
| | | SKRPSGIPERFSGSNSGNTATLTISGTQAEDEADYYCHSYSHRPEVVVFG | |
| | | GGTKLTVL | |
| | VH (DNA) | GAAGTTCAGCTGGTTGAAAGCGGTGGTGGTCTGGTTAAACCTGGTGGTAG | 1009 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTATCTACTCTGGTTTTGATTACTGGGGCCAGGGCACCCTGGTTAC | |
| | | TGTCTCGAGC | |
| | VL (DNA) | AGCTACGAGCTGACACAGCCTCCTAGCGTTTCCGTGTCTCCTGGCCAGAC | 1010 |
| | | AGCCAGCATCACATGTTCTGGCGACAAGCTGGGCGATAAGTACGCCAGCT | |
| | | GGTATCAGCAGAAGCCCGGACAGTCTCCCGTGCTGGTCATCTACCAGGAT | |
| | | AGCAAGAGGCCTAGCGGCATCCCTGAGAGATTCAGCGGCAGCAATAGCGG | |
| | | CAATACCGCCACACTGACAATCAGCGGAACACAGGCCGAGGACGAGGCCG | |
| | | ATTACTACTGTCACAGCTACTCTCACAGACCCGAGGTTGTGGTGTTCGGC | |
| | | GGCGGTACCAAGCTGACAGTGCTG | |
| D-218 | H-CDR1 | NAWMS | 1011 |
| | H-CDR2 | RIKSKTDGGTTDYAAPVKG | 1012 |
| | H-CDR3 | LGIYSGFDY | 1013 |
| | VH (aa) | EVQLVESGGGLVKPGGSLRLSCAASGFTFSNAWMSWVRQAPGKGLEWVGR | 1014 |
| | | IKSKTDGGTTDYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 1015 |
| | L-CDR2 | QDSKRPS | 1016 |
| | L-CDR3 | HSYLHLPPTVV | 1017 |
| | VL (aa) | SYELTQPPSVSVSPGQTASITCSGDKLGDKYASWYQQKPGQSPVLVIYQD | 1018 |
| | | SKRPSGIPERFSGSNSGNTATLTISGTQAEDEADYYCHSYLHLPPTVVFG | |
| | | GGTKLTVL | |
| | VH (DNA) | GAAGTTCAGCTGGTTGAAAGCGGTGGTGGTCTGGTTAAACCTGGTGGTAG | 1019 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTATCTACTCTGGTTTTGATTACTGGGGCCAGGGCACCCTGGTTAC | |
| | | TGTCTCGAGC | |
| | VL (DNA) | AGCTACGAGCTGACACAGCCTCCTAGCGTTTCCGTGTCTCCTGGCCAGAC | 1020 |
| | | AGCCAGCATCACATGTTCTGGCGACAAGCTGGGCGATAAGTACGCCAGCT | |
| | | GGTATCAGCAGAAGCCCGGACAGTCTCCCGTGCTGGTCATCTACCAGGAT | |
| | | AGCAAGAGGCCTAGCGGCATCCCTGAGAGATTCAGCGGCAGCAATAGCGG | |
| | | CAATACCGCCACACTGACAATCAGCGGAACACAGGCCGAGGACGAGGCCG | |
| | | ATTACTACTGTCACAGCTACCTGCATCTGCCCCCTACAGTGGTGTTCGGC | |
| | | GGCGGTACCAAGCTGACAGTGCTG | |
| D-219 | H-CDR1 | NAWMS | 1021 |
| | H-CDR2 | RIKSKTDGGTTDYAAPVKG | 1022 |
| | H-CDR3 | LGIYSGFDY | 1023 |
| | VH (aa) | EVQLVESGGGLVKPGGSLRLSCAASGFTFSNAWMSWVRQAPGKGLEWVGR | 1024 |
| | | IKSKTDGGTTDYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 1025 |
| | L-CDR2 | QDSKRPS | 1026 |
| | L-CDR3 | HAYHWKPTPIVV | 1027 |
| | VL (aa) | SYELTQPPSVSVSPGQTASITCSGDKLGDKYASWYQQKPGQSPVLVIYQD | 1028 |
| | | SKRPSGIPERFSGSNSGNTATLTISGTQAEDEADYYCHAYHWKPTPIVVF | |
| | | GGGTKLTVL | |
| | VH (DNA) | GAAGTTCAGCTGGTTGAAAGCGGTGGTGGTCTGGTTAAACCTGGTGGTAG | 1029 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTATCTACTCTGGTTTTGATTACTGGGGCCAGGGCACCCTGGTTAC | |
| | | TGTCTCGAGC | |
| | VL (DNA) | AGCTACGAGCTGACACAGCCTCCTAGCGTTTCCGTGTCTCCTGGCCAGAC | 1030 |
| | | AGCCAGCATCACATGTTCTGGCGACAAGCTGGGCGATAAGTACGCCAGCT | |
| | | GGTATCAGCAGAAGCCCGGACAGTCTCCCGTGCTGGTCATCTACCAGGAT | |
| | | AGCAAGAGGCCTAGCGGCATCCCTGAGAGATTCAGCGGCAGCAATAGCGG | |
| | | CAATACCGCCACACTGACAATCAGCGGAACACAGGCCGAGGACGAGGCCG | |
| | | ATTACTACTGTCACGCTTACCACTGGAAGCCCACACCTATTGTGGTGTTC | |
| | | GGCGGCGGTACCAAGCTGACAGTGCTG | |
| D-220 | H-CDR1 | NAWMS | 1031 |
| | H-CDR2 | RIKSKTDGGTTDYAAPVKG | 1032 |
| | H-CDR3 | LGIYSGFDY | 1033 |
| | VH (aa) | EVQLVESGGGLVKPGGSLRLSCAASGFTFSNAWMSWVRQAPGKGLEWVGR | 1034 |
| | | IKSKTDGGTTDYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 1035 |
| | L-CDR2 | QDSKRPS | 1036 |
| | L-CDR3 | HTYSHLPPTVV | 1037 |
| | VL (aa) | SYELTQPPSVSVSPGQTASITCSGDKLGDKYASWYQQKPGQSPVLVIYQD | 1038 |
| | | SKRPSGIPERFSGSNSGNTATLTISGTQAEDEADYYCHTYSHLPPTVVFG | |
| | | GGTKLTVL | |
| | VH (DNA) | GAAGTTCAGCTGGTTGAAAGCGGTGGTGGTCTGGTTAAACCTGGTGGTAG | 1039 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTATCTACTCTGGTTTTGATTACTGGGGCCAGGGCACCCTGGTTAC | |
| | | TGTCTCGAGC | |
| | VL (DNA) | AGCTACGAGCTGACACAGCCTCCTAGCGTTTCCGTGTCTCCTGGCCAGAC | 1040 |
| | | AGCCAGCATCACATGTTCTGGCGACAAGCTGGGCGATAAGTACGCCAGCT | |
| | | GGTATCAGCAGAAGCCCGGACAGTCTCCCGTGCTGGTCATCTACCAGGAT | |
| | | AGCAAGAGGCCTAGCGGCATCCCTGAGAGATTCAGCGGCAGCAATAGCGG | |
| | | CAATACCGCCACACTGACAATCAGCGGAACACAGGCCGAGGACGAGGCCG | |
| | | ATTACTACTGTCACACCTACTCTCATCTGCCCCCTACAGTGGTGTTCGGC | |
| | | GGCGGTACCAAGCTGACAGTGCTG | |
| D-221 | H-CDR1 | NAWMS | 1041 |
| | H-CDR2 | RIKSKTDGGTTDYAAPVKG | 1042 |
| | H-CDR3 | LGIYSGFDY | 1043 |
| | VH (aa) | EVQLVESGGGLVKPGGSLRLSCAASGFTFSNAWMSWVRQAPGKGLEWVGR | 1044 |
| | | IKSKTDGGTTDYAAPVKGRFTISRDDSKNTLYLQMNSLKTEDTAVYYCAR | |
| | | LGIYSGFDYWGQGTLVTVSS | |
| | L-CDR1 | SGDKLGDKYAS | 1045 |
| | L-CDR2 | QDSKRPS | 1046 |
| | L-CDR3 | HTYTTLKPSW | 1047 |
| | VL (aa) | SYELTQPPSVSVSPGQTASITCSGDKLGDKYASWYQQKPGQSPVLVIYQD | 1048 |
| | | SKRPSGIPERFSGSNSGNTATLTISGTQAEDEADYYCHTYTTLKPSVVFG | |
| | | GGTKLTVL | |
| | VH (DNA) | GAAGTTCAGCTGGTTGAAAGCGGTGGTGGTCTGGTTAAACCTGGTGGTAG | 1049 |
| | | CCTGCGTCTGAGCTGTGCAGCAAGCGGTTTTACCTTTAGCAATGCATGGA | |
| | | TGAGCTGGGTTCGTCAGGCACCTGGTAAAGGTCTGGAATGGGTTGGTCGT | |
| | | ATTAAAAGCAAAACCGATGGTGGCACCACCGATTATGCAGCTCCGGTTAA | |
| | | AGGTCGTTTTACCATTAGTCGTGATGACAGCAAAAATACCCTGTACCTGC | |
| | | AGATGAATAGCCTGAAAACCGAAGATACGGCCGTCTATTATTGTGCGCGC | |
| | | CTGGGTATCTACTCTGGTTTTGATTACTGGGGCCAGGGCACCCTGGTTAC | |
| | | TGTCTCGAGC | |
| | VL (DNA) | AGCTACGAGCTGACACAGCCTCCTAGCGTTTCCGTGTCTCCTGGCCAGAC | 1050 |
| | | AGCCAGCATCACATGTTCTGGCGACAAGCTGGGCGATAAGTACGCCAGCT | |
| | | GGTATCAGCAGAAGCCCGGACAGTCTCCCGTGCTGGTCATCTACCAGGAT | |
| | | AGCAAGAGGCCTAGCGGCATCCCTGAGAGATTCAGCGGCAGCAATAGCGG | |
| | | CAATACCGCCACACTGACAATCAGCGGAACACAGGCCGAGGACGAGGCCG | |
| | | ATTACTACTGTCACACCTACACCACACTGAAGCCCAGCGTGGTGTTCGGC | |
| | | GGCGGTACCAAGCTGACAGTGCTG | |
| D-222 | H-CDR1 | HAWIS | 1051 |
| | H-CDR2 | QIKGGPGSGGTSYAEPVKG | 1052 |
| | H-CDR3 | LGIYSGFDY | 1053 |
| | VH (aa) | EVQLVESGGGLVKPGGSLRLSCAASGFTFSHAWISWVRQAPGKGLEWVGQ | 1054 |
| | | IKGGPGSGGTSYAEPVKGRFTISRDDSKNTLYLQMNSLKTEDTAVYYCAR | |
| | | LGIYSGFDYWGQGTLVTVSS | |
| | L-CDR1 | SGDKLGDKYAS | 1055 |
| | L-CDR2 | QDSKRPS | 1056 |
| | L-CDR3 | HTYSHRPEIVV | 1057 |
| | VL (aa) | SYELTQPPSVSVSPGQTASITCSGDKLGDKYASWYQQKPGQSPVLVIYQD | 1058 |
| | | SKRPSGIPERFSGSNSGNTATLTISGTQAEDEADYYCHTYSHRPEIVVFG | |
| | | GGTKLTVL | |
| | VH (DNA) | GAAGTGCAGCTGGTTGAATCTGGCGGCGGACTGGTTAAGCCTGGCGGATC | 1059 |
| | | TCTGAGACTGAGCTGTGCCGCCAGCGGCTTCACATTTTCTCACGCCTGGA | |
| | | TCAGCTGGGTCCGACAGGCTCCTGGAAAAGGCCTGGAATGGGTCGGACAG | |
| | | ATCAAAGGCGGACCTGGCTCTGGCGGAACAAGCTATGCCGAGCCTGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGTGCCAGA | |
| | | CTGGGCATCTACTCCGGCTTCGATTATTGGGGCCAGGGCACCCTGGTTAC | |
| | | AGTCTCGAGC | |
| | VL (DNA) | AGCTACGAGCTGACACAGCCTCCAAGCGTGTCCGTGTCTCCTGGACAGAC | 1060 |
| | | AGCCAGCATCACCTGTAGCGGCGATAAGCTGGGCGATAAGTACGCCAGCT | |
| | | GGTATCAGCAGAAGCCCGGCCAGTCTCCTGTGCTGGTCATCTACCAGGAC | |
| | | AGCAAGAGGCCTAGCGGCATCCCCGAGAGATTCAGCGGCAGCAATAGCGG | |
| | | CAATACCGCCACACTGACCATCAGCGGAACACAGGCCGAGGACGAGGCCG | |
| | | ATTACTACTGCCACACCTACAGCCACCGGCCTGAGATCGTGGTTTTTGGC | |
| | | GGAGGTACCAAGCTGACAGTGCTG | |
| D-223 | H-CDR1 | HAWIS | 1061 |
| | H-CDR2 | QIKGGPGSGGTSYAEPVKG | 1062 |
| | H-CDR3 | LGIYSGFDY | 1063 |
| | VH (aa) | EVQLVESGGGLVKPGGSLRLSCAASGFTFSHAWISWVRQAPGKGLEWVGQ | 1064 |
| | | IKGGPGSGGTSYAEPVKGRFTISRDDSKNTLYLQMNSLKTEDTAVYYCAR | |
| | | LGIYSGFDYWGQGTLVTVSS | |
| | L-CDR1 | SGDKLGDKYAS | 1065 |
| | L-CDR2 | QDSKRPS | 1066 |
| | L-CDR3 | HSYLHLPPTVV | 1067 |
| | VL (aa) | SYELTQPPSVSVSPGQTASITCSGDKLGDKYASWYQQKPGQSPVLVIYQD | 1068 |
| | | SKRPSGIPERFSGSNSGNTATLTISGTQAEDEADYYCHSYLHLPPTVVFG | |
| | | GGTKLTVL | |
| | VH (DNA) | GAAGTGCAGCTGGTTGAATCTGGCGGCGGACTGGTTAAGCCTGGCGGATC | 1069 |
| | | TCTGAGACTGAGCTGTGCCGCCAGCGGCTTCACATTTTCTCACGCCTGGA | |
| | | TCAGCTGGGTCCGACAGGCTCCTGGAAAAGGCCTGGAATGGGTCGGACAG | |
| | | ATCAAAGGCGGACCTGGCTCTGGCGGAACAAGCTATGCCGAGCCTGTGAA | |
| | | GGGCAGATTCACCATCAGCCGGGACGACAGCAAGAACACCCTGTACCTGC | |
| | | AGATGAACAGCCTGAAAACCGAGGACACCGCCGTGTACTACTGTGCCAGA | |
| | | CTGGGCATCTACTCCGGCTTCGATTATTGGGGCCAGGGCACCCTGGTTAC | |
| | | AGTCTCGAGC | |
| | VL (DNA) | AGCTACGAGCTGACACAGCCTCCTAGCGTTTCCGTGTCTCCTGGCCAGAC | 1070 |
| | | AGCCAGCATCACATGTTCTGGCGACAAGCTGGGCGATAAGTACGCCAGCT | |
| | | GGTATCAGCAGAAGCCCGGACAGTCTCCCGTGCTGGTCATCTACCAGGAT | |
| | | AGCAAGAGGCCTAGCGGCATCCCTGAGAGATTCAGCGGCAGCAATAGCGG | |
| | | CAATACCGCCACACTGACAATCAGCGGAACACAGGCCGAGGACGAGGCCG | |
| | | ATTACTACTGTCACAGCTACCTGCATCTGCCCCCTACAGTGGTGTTCGGC | |
| | | GGCGGTACCAAGCTGACAGTGCTG | |

TABLE 13.4
EC50 (nM) of binding (FACS) of IgG-format ABPs of
maturated ABPs of this Example to cell lines.

| | Cell-binding (FACS) | | | |
|---|---|---|---|---|
| | | MDA-MB-231- | MC38- | |
| | Colo741 | IGSF11 | IGSF11 | |
| Antibody | (human) | (human) | (murine) | MC38-wt |
| D-114 | <0.5 | | | <0.5 |
| D-115 | <0.1 | | | |
| D-116 | <0.5 | | | <0.1 |
| D-222 | <0.5 | <0.5 | <0.5 | |
| D-223 | <0.5 | | | <0.5 |

### EXAMPLE 14: APPARENT AFFINITY OF ABPS

The inventors determined the apparent affinity of various ABPs described herein (and/or those disclosed in WO 2018/027042 A1) (eg, in IgG format), including those described in Example 13, by bioloayer interferometry (BLI) on a ForteBio OctetRed96e (Table 14). Briefly, various APBs were loaded on an optical biosensor via a commercially available Capture System (AHC sensors, ForteBio). The test APBs on the biosensor surface were bound by IGSF11 (ECD domain) at a single concentration (100 nM), and the apparent affinity was determined from analysis of the resulting association/dissociations curves of IGSF11 (ECD domain).

TABLE 14
Apparent affinity of ABPs to IGSF11,
including those of Example 13

| Antibody | Affinity (nM) |
|---|---|
| A-006 | <15 |
| C-001 | <75 |
| C-003 | <15 |
| C-005 | <15 |
| C-007 | <125 |
| C-016 | n.d. |
| C-017 | weak |

n.d. = not determinable

The inventors demonstrated that they could significantly improve the affinity of APBs of the invention that bind to the IGC2 domain of IGSF11 by maturation. Surprising high affinities could be detected for the maturated APBs, in particular for the APBs D-114 and D-222 (Table 14.1). Indeed, the affinity of APB D-114 was too high to be measured with a binding curve using a CBP concentration below the KD and only allowed a KD estimation based on repeated affinity measurement at 15 pM CBP.

TABLE 14.1
Affinity of maturated ABPs of Example 13 to IGSF11

| | Affinity (KD) | | |
|---|---|---|---|
| Antibody | Best fit KD | CBP concentration | Analysis |
| D-114 | ND, <10 pM (est) | 15 pM | Single binding curve |
| D-222 | <150 pM | 80 pM, 800 pM | N-curve |

ND = not determinable

Solution-based binding affinities were determined using the Kinetic Exclusion Assay (KinExA) 4000 system as described below, where binding partners were combined and allowed to reach equilibrium prior to measurement.

Briefly, anti-IGSF11 ABPs of the invention as Fabs were used as Constant Binding Partner (CBP) and human recombinant IGSF11 ECD was used as Titrant. After reaching equilibrium, free CBP were detected using PMMA beads coated with IGSF11 ECD or IGSF11 IgC2-domain Fc-fusion protein and an anti-human F(ab′)2 antibody conjugated with AlexaFluor647. CBP concentrations above and below the expected KD (95% CI) of each molecule were selected to provide a full concentration response. The CBP was incubated with different Titrant concentrations for 16 h (or 72-90 h) to reach equilibrium. The equilibrium binding curves of different CBP concentrations were measured in duplicates and drift correction was applied. The n-curve analysis tool within the KinExA Pro Software version 4.4.26 was applied to obtain one KD value per set of binding curves by finding the best fit of a 1:1 binding model to the data.

### EXAMPLE 15: ABPS BINDING TO THE IGC2 DOMAIN OR THE IGV DOMAIN OF IGSF11, AND INHIBITION OF THE INTERACTION BETWEEN IGSF11 AND VSIR

The inventors identified that certain anti-IGSF11 ABPs bind to the IgC2 domain of IGSF11, and others bind to the IgV domain of IGSF11. Table 15.1 of ELISA data showing that despite all IgG antibodies tested were confirmed to bind the full-length ECD of IGSF11 (FIG. 17A), there were those that bound to the IgC2 domain of IGSF11 (FIG. 17B) and others that bound to the IgV domain of IGSF11 (FIG. 17C).

The inventors showed that, surprisingly, the ability of an ABP to inhibit the interaction between IGSF11 and VSIR (eg, as determined according to Comparative Example 5) was associated with such ABP binding to the IgC2 domain of IGSF11, and not to the IgV domain of IGSF11. Correspondingly, ABPs binding to the IgV domain of IGSF11 are not associated with inhibition of the interaction between IGSF11 and VSIR.

The inhibition of IGSF11 binding to VSIR was tested as follows: Recombinant purified human VSIR-Fc (human IgG1) (R&D Systems, Cat #7126-B7) was immobilised on an ELISA plate (Nunc MaxiSorp) at 2 ug/mL (in PBS), and the plates were then washed and blocked with 2% BSA in PBS/Tween (0.05%); a dilution series of anti-IGSF11 ABPs (IgG), or control IgG antibody of irrelevant specificity, was pre-incubated with 200 nM IGSF11 ECD (his-tagged, SinoBiological, Cat #13094-H08H) for 30 minutes; IGSF11-antibody complexes were added to the immobilised VSIR-Fc for binding, and plates were then washed to remove unbound IGSF11 ECD; IGSF11 ECD bound to immobilized VSIR-Fc was detected with a horseradish peroxidase-conjugated goat anti-hexahistidine antibody (Abcam, Cat #Ab1269), and after washing the ELISA signal was developed with 3,3,5,5′-Tetramethylbenzidine (TMB) substrate. All binding steps were for 1 hour at room temperature, and all washing steps were three times washing with PBS/Tween (0.05%).

TABLE 15.1
EC50 (nM) of binding of ABPs of the Comparative Examples
to the full-length ECD, IgC2 domain and IgV domain of IGSF11.

| | | | | | Inhibition: |
|---|---|---|---|---|---|
| | | IgC2 | IgV | Domain | IGSF11- |
| Antibody | ECD | domain | domain | specificity | VSIR |
| A-006 | <1 | <1 | n.d. | C2 | +++ |
| A-022 | <1 | <1 | n.d. | C2 | +++ |
| A-011 | <1 | <1 | n.d. | C2 | +++ |
| A-027 | 1-20 | 1-20 | n.d. | C2 | ++ |
| A-026 | 1-20 | 1-20 | n.d. | C2 | +++ |
| A-035 | 1-20 | 20-100 | n.d. | C2 | ++ |
| A-024 | <1 | n.d. | <1 | V | − |
| A-020 | <1 | n.d. | <1 | V | − |
| A-010 | <1 | n.d. | <1 | V | − |
| A-004 | 1-20 | n.d. | 1-20 | V | − |

n.d. not determinable
− = no inhibition; + = inhibition; ++ = medium inhibition; +++ = strong inhibition

The further ABPs of Example 13 are similarly tested for domain specificity by ELISA, and inhibition of IGSF11-binding by VISIR was tested by BLI. Briefly, biotinylated IGSF11 (or domain thereof) is loaded on an optical biosensor via a streptavidin surface. The IGSF11 (or domain thereof) on the biosensor surface is bound by the ABP and simultaneous binding of the VSIR multimer is tested. The multimer of VSIR protein (“VISTA.COMP”, a stable pentameric construct of the IgV domain of VSIR (VISTA) fused to the pentamerization domain from the cartilage oligomeric matrix protein (COMP)) was described in Prodeus et al 2017, JCI Insight 2 (18):e94308. The inhibition of the interaction between IGSF11 and VSIR is further associated with such ABPs that bind to the IgC2 domain of IGSF11 (Table 15.2). In particular, it is found that those ABPs of Example 13 that bind to the IgC2 domain of IGSF11 inhibit the binding between IGSF11 and VSIR. In contrast, those ABPs of Example 13 found to bind to the IgV domain of IGSF11 lack the ability to inhibit the binding between IGSF11 and VSIR.

TABLE 15.2
EC50 (nM) of binding of ABPs of Example 13 to the
full-length ECD, IgC2 domain and IgV domain of IGSF11.

| | | | | Domain |
|---|---|---|---|---|
| Antibody | ECD | IgC2 | IgV | specificity |
| C-001 | <1 | n.d. | <1 | V |
| C-002 | 1-20 | <1 | n.d. | C2 |
| C-003 | <1 | <1 | n.d. | C2 |
| C-004 | | | | C2 |
| C-005 | <1 | <1 | n.d. | C2 |
| C-006 | 1-20 | <1 | n.d. | C2 |
| C-007 | <1 | n.d. | <1 | V |
| C-008 | <1 | n.d. | <1 | V |
| C-009 | 1-20 | n.d. | <1 | V |
| C-010 | 20-100 | 1-20 | n.d. | C2 |
| C-011 | <1 | <1 | n.d. | C2 |
| C-012 | | | | NT |
| C-013 | 1-20 | <1 | n.d. | C2 |
| C-014 | 1-20 | <1 | n.d. | C2 |
| C-015 | 20-100 | 1-20 | n.d. | C2 |
| C-016 | 1-20 | n.d. | 1-20 | V |
| C-017 | 1-20 | n.d. | 1-20 | V |
| C-018 | 1-20 | <1 | n.d. | C2 |
| C-019 | | | | NT |
| C-020 | | | | NT |
| C-021 | <1 | <1 | n.d. | C2 |
| C-022 | 1-20 | 1-20 | n.d. | C2 |
| C-023 | <1 | <1 | n.d. | C2 |
| C-024 | 1-20 | n.d. | 1-20 | V |
| C-025 | 1-20 | n.d. | <1 | V |
| C-026 | 1-20 | n.d. | <1 | V |
| C-027 | | | | NT |
| C-028 | | | | NT |
| C-029 | | | | NT |

n.d. not determinable; NT = not tested

The IGSF11 domain specificity of the ABPs described herein was further supported by biolayer interferometry (BLI) experiments on a ForteBio OctetTed96e (FIG. 17D) to test for binding between A-006 (“A-006-like”) or A-024 (“A-024-like”) ABPs (in IgG1 format) to either extra-cellular, to IgV or to IgC2 domain of IGSF11. As shown in FIG. 17D the A-006-like IgG1 (left column) binds to the entire ECD of IGSF11 (top row) and to the IgC2 domain of IGSF11 (bottom row) but does not bind to the IgV domain of IGSF11 (middle row). In contrast, the A-024-like IgG1 (right column) binds to the entire ECD of IGSF11 (top row) and to the IgV domain of IGSF11 (middle row) but does not bind to the IgC2 domain of IGSF11 (bottom row).

Indeed, and surprisingly, the inventors demonstrated that it is the IgC2 domain of IGSF11 that interacts with the VSIR protein (FIG. 17E). In a BLI assay analogous to the preceding assay, a VSIR multimer protein was tested for binding to the either extra-cellular, to IgV and to IgC2 domain of IGSF11. Surprisingly, this VSIR-multimer binds to the entire ECD of IGSF11 (top row) and to the IgC2 domain of IGSF11 (bottom row) but does not bind to the IgV domain of IGSF11 (middle row). Briefly, IGSF11 fused to murine-Fc (either the ECD or domains thereof) was loaded on an optical biosensor via a capture system (CaptureSelect, ThermoFisher. Binding of IGSF11 (ECD or domains thereof) on the biosensor surface by either the A-006-like/A-024-likeABPs (FIG. 17D) or the the VSIR multimer was tested.

IgC2 and IgV domains of IGSF11 were separately produced as murine-Fc (mFc) fusions, briefly as follows:

Human IGSF11 IgV-like (amino acids 23-143; SEQ ID NO. 388) and IgC2-like amino acids 137-241; SEQ ID NO. 389) domain sequences (amino acids with reference to UNIPROT identifier Q5DX21-1/ISOFORM 1/SEQ ID 371) were genetically fused to a murine IgG2a Fc-region. Expi293 cells (ThermoFisher) were transiently transfected with DNA sequences encoding the applicable IGSF11 domain-mFc fusions according to the manufacturer's instructions, and cultured under conditions suitable to the IGSF11 domain fusion. Cell supernatants were harvested 5 days post transfection and the expressed Fc-protein purified via Protein A affinity chromatography (GE Healthcare). IGSF11 domain Fc-fusion proteins were re-buffered into PBS pH7.4. Binding of IgG antibodies to each of the full-length ECD of IGSF11, to the IgV domain of IGSF11 and to the IgC2 domain of IGSF11, was tested in the ELISA assay as follows: briefly, recombinant domains of Fc-fusion IGSF11 proteins were coated at 2 ug/mL on a 384-well Maxisorp plate. The surface was blocked with 2% (w/v) skim milk powder in PBST. After three wash cycles with PBST, a dilution series of IgG-format ABPs was transferred to the immobilized the Fc-fusion IGSF11 ECD or the IGSF11 domains and incubated for 1h. After removing all unbound antibodies by 3 wash cycles with PBST, bound IgG ABPs were detected with a goat anti-human IgG antibody conjugated with horseradish peroxidase. After three wash cycles with PBST the ELISA was developed with TMB substrate.

Generally, A-006 (“A-006-like”) ABPs, including the ABPs of Example 13: C-002, C-003, C-004, C-005, C-006, C-010, C-011, C-013, C-014, C-015, C-018, C-021, C-022, and C-023, are found to bind the IgC2 domain (FIG. 18A) and inhibit the interaction between IGSF11 and VSIR, shown by either bio-layer interferometry (BLI) with bound IGSF11 (FIG. 18B) or ELISA with bound VSIR (FIG. 19). In contrast, A-024 (“A-024-like”), ABPs including the ABPs of Example 13: C-001, C-007, C-008, C-009, C-016, C-017, C-024, C-025, and C-026), are concluded to bind the IgV domain (FIG. 18C) but do not inhibit the interaction between IGSF11 and VISTA, shown by either bio-layer interferometry (BLI) with bound IGSF11 (FIG. 18D) or ELISA with bound VSIR (FIG. 19). In particular, ABP C-004 that binds to the IgC2 domain of IGSF11 was shown in this assay to compete with VSIR for binding to surface-bound IGSF11 (FIG. 18E, upper), as was shown for other IgC2 domain-binding ABPs disclosed herein including A-006 and C-005 (data not shown). In contrast, ABP C-001 that binds to the IgV domain of IGSF11 was shown in this assay to not compete with VSIR for binding to surface-bound IGSF11 (FIG. 18E, lower).

Certain ABPs disclosed herein were investigated by cross-competition for epitope binning. Results from this epitope binning further supports the two different groups of IGSF11-binding ABPs (Table 15.3). Such binning experiments were performed by biolayer interferometry on a ForteBio OctetRed96e. Briefly, biotinylated IGSF11 was loaded on an optical biosensor via a streptavidin surface. IGSF11 on the biosensor surface was bound by the primary ABP and simultaneous binding of a secondary ABP was tested. Other IGSF11-binding ABPs (such as those disclosed in WO 2018/027042 A1) are similarly tested for cross competition with ABPs of the Comparative Examples or the Examples.

TABLE 15.3
Cross competition of two groups of ABPS

| Primary | Secondary ABP | | | | | | | |
|---|---|---|---|---|---|---|---|---|
| ABP | A-006 | C-001 | C-005 | C-003 | C-004 | C-007 | C-016 | C-017 |
| A-006 | Competing | Not | Competing | Competing | Competing | Not | Not | Not |
| C-001 | Not | Competing | Not | Not | Not | Competing | Competing | Competing |

For several APBs described herein (eg C-001, D-114 and D-222) a surprisingly strong IgG binding response could be demonstrated by the inventors. However, as described above, inhibition of binding between IGSF11 and VISTA could only be detected for those APBs that bound to the IGC2 domain of IGSF11 (eg D-114 and D-222; FIGS. 28 A & B). Accordingly, for C-001, the ABP that binds to the IgV domain of IGSF11, no inhibition of IGSF11/VISTA binding could be observed (FIG. 28 C).

Competition experiments with recombinant VISTA were performed by biolayer interferometry on a ForteBio OctetRed96e. Briefly, biotinylated IGSF11 was loaded on an optical biosensor via a streptavidin surface. The IGSF11-loaded biosensor was dipped into the samples with different concentrations of anti-IGSF11 ABP, and ABPs were bound for 600s. Simultaneous binding of a VISTA multimer (pentameric VISTA.COMP) was tested by dipping the biosensors with pre-complexed IGSF11 into the VISTA samples. The additional binding response of VISTA was analyzed after 400s. The binding response of simultaneously binding VISTA was normalized to the binding response of VISTA without prior antibody binding.

### EXAMPLE 16: A-006-LIKE ABPS THAT BIND THE IGC2 DOMAIN OF IGSF11 SHOW ENHANCED T CELL-MEDIATED TUMOUR CELL KILLING

The Inventors show that, unlike (IgV-binding) A-024-like ABPs, A-006-like ABPs that bind the IgC2 domain of IGSF11 show substantial, dose-dependent and robust T cell-mediated killing of a tumour cell line (MDA-MB-231) engineered to over express IGSF11 (FIG. 20A), using T cells from a healthy donor. This tumour cell killing correlated with T cell activation, shown by expression of CD69 (FIG. 20B). Of note is that these functional differences between the IgC2 domain-binding and the IgV domain-binding ABPs is not an affinity affect: although the IgV domain-binding A-024-like ABP (triangles) binds to cells with a better affinity than the IgC2 domain-binding A-006-like ABP (squares)—for example as shown using a FACS binding assay to determine the EC50 of binding of antibodies to lung cancer cell line DMS 273, analogous to Comparative Example 6 (Table 6.1)—it is the IgC2 domain-binding A-006-like ABP that shows substantially enhanced tumour cell killing in this assay.

Indeed, the T cell-mediated tumour cell killing that is induced by an IgC2 domain-binding A-006-like ABP was abolished by increasing concentrations of soluble ECD His-tagged IGSF11; by competing for binding of the A-006-like ABP with the tumour cell-expressed IGSF11 (FIG. 21A). Furthermore, increasing concentrations of soluble ECD His-tagged IGSF11 inhibited tumour cell-binding of the A-006-like ABP (FIG. 21B).

This anti-EpCam×CD3 “BiTE”-based assay was conducted, generally as described in Comparative Example 7 to generate FIG. 14, except that tumour cell lysis was monitored using CellTiter Glo instead of by a luciferase read-out.

Generally, the assay is described in more detail in the following For this assay, 6,000 IGSF11-expressing MDA-MB-231-luc cells were seeded into every well of a flat bottom 96-well plate and incubated for 24h. Then 1×10\hat{ }5 naive CD3+ T cells (freshly isolated from PBMCs) were added and co-cultured with the tumour cells in the presence of 2 ng/mL EpCAM×CD3 BiTE (solitomab, AmGen) and the ABPs (A-006-like and A-024-like), either in increasing concentrations (0.32-200 ug/ml, FIGS. 20A & B) or at 20 ug/ml (FIGS. 21A & B). To block the IGSF11-specific tumour cell binding of the A-006-like ABP, increasing concentrations of recombinant human IGSF11-ECD-His protein (Sino Biologicals) were added to the assay wells (1.56-25 ug/mL, FIGS. 21A & B). After 3 days of co-culture tumour cell lysis was monitored using CellTiter Glo (Promega) read-out (FIGS. 20A & 21A), T cells were analysed for T cell activation marker expression via flow cytometry (FIG. 20B) and assay supernatants were tested for their binding capacity to IGSF11+ tumour cells via flow cytometry (FIG. 21B).

For measuring tumour cell lysis using CellTiter Glo, either the T cells (FIG. 20A) or the supernatants (FIG. 21A) were removed, and the tumour cells in the assay plate was washed once with PBS. PBS was removed and 50 uL of fresh medium was added together with 50 uL of freshly prepared CellTiter Glo reagent (Promega). The plate was incubated for 2 min on a plate shaker. Then the supernatants were transferred to a new 96-well white flat-bottom plate and incubated for another 10 min to stabilize the luminescent signal. Luminescence was then measured at a Tecan Spark 20M plate reader.

For T cell activation marker expression analysis (FIG. 20B), the collected T cells were washed once with FACS buffer (PBS+3% FBS), triplicate wells were pooled and subsequently stained using anti-CD3-APC and anti-CD69-BV711 FACS antibodies or respective isotype control antibodies (all from Biolegend). T cells were incubated with the FACS antibodies for 30 min on ice in the dark. Cells were then washed three times with 150 uL FACS buffer, finally diluted in FACS buffer containing 7-AAD live/dead cell marker and measured using an iQue Screener Plus (IntelliCyt) flow cytometer. FACS data analysis of 7-AAD-CD3+CD69+ cells was conducted using FlowJo software.

For cell binding analysis (FIG. 21B), the assay supernatants from triplicate wells were pooled. Supernatants were cooled at 4° C. for 15 min prior to staining. 5×10∝fresh MDA-MB-231-luc/IGSF11 cells were seeded into a 96-well V-bottom plate. The plate was centrifuged, supernatants were removed and 50 uL of pre-cooled assay supernatants were added to the cells and incubated for 1h on ice. Then the cells were washed three times with 150 uL FACS buffer and the cell supernatants were removed. Cells were then resuspended in 50 uL of a 1:80 dilution of secondary antibody (Alexa Fluor® 647 anti-human IgG Fc; Biolegend) in FACS buffer. Secondary antibody was incubated for 30 min on ice in the dark. Cells were then washed three times with 150 uL FACS buffer, finally diluted in FACS buffer containing 7-AAD live/dead cell marker and measured using an iQue Screener Plus (IntelliCyt) flow cytometer. FACS data analysis of 7-AAD-IGSF11+ cells was conducted using FlowJo software.

### EXAMPLE 17: A-006-LIKE ABPS THAT BIND THE IGC2 DOMAIN OF IGSF11 SHOW ENHANCED T CELL MEDIATED KILLING OF CELLS OF A WILD-TYPE TUMOUR CELL LINE

The inventors show that A-006-like ABPs that bind the IgC2 domain of IGSF11 show substantially enhanced T cell-mediated killing of a tumour cell line that naturally expresses IGSF11 (COLO-741) compared to A-024-like ABPs that bind the IgV domain of IGSF11, or compared to isotype control ABP (Ref001). Indeed, this enhanced T cell-mediated tumour cell killing is shown in the absence of any T cell engaging bispecific Bite (FIG. 22A).

Surprisingly, in this assay COLO-741 cells were found not to be sensitive to exposure to an anti-PDL1 antibody (despite PDL1 expression), yet were still sensitive to the IgC2 domain binding A-006-like ABPs, indicating that ABPs binding to the IgC2 domain of IGSF11 have particular utility to treat cancers that are resistant to anti-PDL1 (or anti PD1) therapy (FIG. 22). The respective antigen of each antibody used was detected on the surface of the Colo-741 cells, as demonstrated by FACS staining (FIG. 22B).

This assay was conducted, generally as the “BiTE” assay described in Example 16, except that instead of the MDA-MB-231 cell line engineered to over express IGSF11, a tumour cell line that naturally express IGSF11 (COLO-471) was used, and that the anti EpCam×CD3 “BiTE” was not included (as the tumour cell line did not express EpCam, data not shown).

Briefly as described in the following: 15,000 COLO-741 cells were seeded into a flat bottom 96-well plate and incubated for 24h. Then 1×10\hat{ }5 human naive CD3+ T cells (freshly isolated from PBMCs of healthy donors) were added and co-cultured with the tumour cells in the presence of 40 ug/mL of the A-006-like or A-024-like ABPs, the anti-PDL1 antibody (atezolizumab), or isotype control antibody, respectively. After 3 days of co-culture tumour cell lysis was monitored using CellTiter Glo (Promega) read-out as described above for FIG. 21A.

### EXAMPLE 18: TUMOUR CELL KILLING BY IGC2 DOMAIN BINDING A-006-LIKE ABPS IS MEDIATED BY THE PRESENCE OF AND CONTACT BY T CELLS

The inventors show that tumour cell killing by A-006-Ike ABPs that bind the IgC2 domain of IGSF11 requires the presence of (ie, the contact by) T cells, and not just the addition of supernatant from cytotoxic T cells (FIG. 23). Briefly, 15,000 COLO-741 cells were seeded into a flat bottom 96-well plate and incubated for 24h. Then either increasing concentrations of A-006-like and A-024-like ABP (antibody only) were added, or 1×10\hat{ }5 naive CD3+ T cells (freshly isolated from PBMCs) were added and co-cultured with the tumour cells in the presence of increasing concentrations of the A-006-like or A-024-like ABPs (antibody plus T cells), or increasing concentrations of the A-006-like or A-024-like ABPs, plus 50 uL of CD3/CD28-bead activated T cell supernatants were added (antibody plus T cell supernatants). After 3 days of co-culture tumour cell lysis was monitored using CellTiter Glo (Promega) read-out as described above.

Activated T cell supernatants were generated by incubating isolated CD3+ T cells at a density of 2×10\hat{ }6 cells/well with CD3/CD28 Dynabeads (Invitrogen) at a cell:bead ratio of 1:1. After 48h of incubation, Dynabeads were removed from the T cells by magnetic separation and T cells were pelleted by 10 min centrifugation at 300×g. The cell-free T cell supernatant was then aliquoted and stored at −20° C. for later use.

The sequences show:

SEQ ID NOs. 1 to 370 (Amino acid sequences of CDR and variable regions of ABPs of the Comparative Example 3, as well as nucleic acid sequences encoding variable regions of ABPs of the Comparative Example 3): See Table 1A.

```
SEQ ID NO. 371
(Human IGSF11 protein isoform 1; UniProt identifier Q5DX21-1):
                10 20 30 40 50
MTSQRSPLAP LLLLSLHGVA ASLEVSESPG SIQVARGQPA VLPCTFTTSA
                60 70 80 90 100
ALINLNVIWM VTPLSNANQP EQVILYQGGQ MFDGAPRFHG RVGFTGTMPA
              110 120 130 140 150
TNVSIFINNT QLSDTGTYQC LVNNLPDIGG RNIGVTGLTV LVPPSAPHCQ
              160 170 180 190 200
IQGSQDIGSD VILLCSSEEG IPRPTYLWEK LDNTLKLPPT ATQDQVQGTV
              210 220 230 240 250
TIRNISALSS GLYQCVASNA IGTSTCLLDL QVISPQPRNI GLIAGAIGTG
              260 270 280 290 300
AVIIIFCIAL ILGAFFYWRS KNKEEEEEEI PNEIREDDLP PKCSSAKAFH
              310 320 330 340 350
TEISSSDNNT LTSSNAYNSR YWSNNPKVHR NTESVSHFSD LGQSFSFHSG
              360 370 380 390 400
NANIPSIYAN GTHLVPGQHK TLVVTANRGS SPQVMSRSNG SVSRKPRPPH
              410 420 430
THSYTISHAT LERIGAVPVM VPAQSRAGSL V
SEQ ID NO. 372
(Human IGSF11 protein isoform 2; UniProt identifier Q5DX21-2):
                10 20 30 40 50
MSLVELLLWW NCFSRTGVAA SLEVSESPGS IQVARGQPAV LPCTFTTSAA
                60 70 80 90 100
LINLNVIWMV TPLSNANQPE QVILYQGGQM FDGAPRFHGR VGFTGTMPAT
              110 120 130 140 150
NVSIFINNTQ LSDTGTYQCL VNNLPDIGGR NIGVTGLTVL VPPSAPHCQI
              160 170 180 190 200
QGSQDIGSDV ILLCSSEEGI PRPTYLWEKL DNTLKLPPTA TQDQVQGTVT
              210 220 230 240 250
IRNISALSSG LYQCVASNAI GTSTCLLDLQ VISPQPRNIG LIAGAIGTGA
              260 270 280 290 300
VIIIFCIALI LGAFFYWRSK NKEEEEEEIP NEIREDDLPP KCSSAKAFHT
              310 320 330 340 350
EISSSDNNTL TSSNAYNSRY WSNNPKVHRN TESVSHFSDL GQSFSFHSGN
              360 370 380 390 400
ANIPSIYANG THLVPGQHKT LVVTANRGSS PQVMSRSNGS VSRKPRPPHT
              410 420 430
HSYTISHATL ERIGAVPVMV PAQSRAGSLV
SEQ ID NO. 373
(Human IGSF11 protein isoform 3; UniProt identifier Q5DX21-3):
                10 20 30 40 50
MSLVELLLWW NCFSRTGVAA SLEVSESPGS IQVARGQPAV LPCTFTTSAA
                60 70 80 90 100
LINLNVIWMV TPLSNANQPE QVILYQGGQM FDGAPRFHGR VGFTGTMPAT
              110 120 130 140 150
NVSIFINNTQ LSDTGTYQCL VNNLPDIGGR NIGVTGLTVL VPPSAPHCQI
              160 170 180 190 200
QGSQDIGSDV ILLCSSEEGI PRPTYLWEKL DNTLKLPPTA TQDQVQGTVT
              210 220 230 240 250
IRNISALSSA QPRNIGLIAG AIGTGAVIII FCIALILGAF FYWRSKNKEE
              260 270 280 290 300
EEEEIPNEIR EDDLPPKCSS AKAFHTEISS SDNNTLTSSN AYNSRYWSNN
              310 320 330 340 350
PKVHRNTESV SHFSDLGQSF SFHSGNANIP SIYANGTHLV PGQHKTLVVT
              360 370 380 390 400
ANRGSSPQVM SRSNGSVSRK PRPPHTHSYT ISHATLERIG AVPVMVPAQS
RAGSLV
SEQ ID NO. 374
(ECD of human IGSF11 protein; UniProt identifier Q5DX21):
                10 20 30 40 50
LEVSESPGSI QVARGQPAVL PCTFTTSAAL INLNVIWMVT PLSNANQPEQ
                60 70 80 90 100
VILYQGGQMF DGAPRFHGRV GFTGTMPATN VSIFINNTQL SDTGTYQCLV
              110 120 130 140 150
NNLPDIGGRN IGVTGLTVLV PPSAPHCQIQ GSQDIGSDVI LLCSSEEGIP
              160 170 180 190 200
RPTYLWEKLD NTLKLPPTAT QDQVQGTVTI RNISALSSGL YQCVASNAIG
              210
TSTCLLDLQV ISPQPRNIG
SEQ ID NO. 375
(Ig-like V-type domain of human IGSF11 protein; UniProt identifier Q5DX21):
                10 20 30 40 50
LEVSESPGSI QVARGQPAVL PCTFTTSAAL INLNVIWMVT PLSNANQPEQ
                60 70 80 90 100
VILYQGGQMF DGAPRFHGRV GFTGTMPATN VSIFINNTQL SDTGTYQCLV
              110
NNLPDIGGRN IGVT
SEQ ID NO. 376
(Ig-like C2-type domain of human IGSF11 protein; UniProt identifier Q5DX21):
                10 20 30 40 50
PSAPHCQIQG SQDIGSDVIL LCSSEEGIPR PTYLWEKLDN TLKLPPTATQ
                60 70 80 90
DQVQGTVTIR NISALSSGLY QCVASNAIGT STCLLDLQVI S
SEQ ID NO. 377
(Cynomolgus monkey IGSF11 protein; UniProt identifier G7NXN0):
                10 20 30 40 50
MTSRRSPLAP LLLLSLHGVA ASLEVSESPG SIQVARGQTA VLPCTFTTSA
                60 70 80 90 100
ALINLNVIWM VTPLSNANQP EQVILYQGGQ MFDGAPRFHG RVGFTGTMPA
              110 120 130 140 150
TNVSVFINNT QLSDTGTYQC LVNNLPDIGG RNIGVTGLTV LVPPSAPHCQ
              160 170 180 190 200
IQGSQDIGSD VILLCSSEEG IPRPTYLWEK LDNTLKLPPT ATQDQVQGTV
              210 220 230 240 250
TIRNISTLTS GLYQCVASNA IGTSTCLLDL QVISPQPRNI GLIAGAVGTG
              260 270 280 290 300
AVIIIFCIAL ILGAFFYWRS KNKEEEEEEI PNEIREDDLP PKCSSAKAFH
              310 320 330 340 350
TEISSSDNNT LTSSNTYNSR YWSNNPKVHR NTESVNHFSD LGQSFSLRSG
              360 370 380 390 400
NASIPSIYAN GSHLLPGQHK TLVVTANRGS SPQVMSRSNG SVSRKPRPPH
              410 420 430
SHSYTISHAT LERIGAVPVM VPAQSRAGSL V
SEQ ID NO. 378
(Murine IGSF11 protein; UniProt identifier P0C673):
                10 20 30 40 50
MTRRRSAPAS WLLVSLLGVA TSLEVSESPG SVQVARGQTA VLPCAFSTSA
                60 70 80 90 100
ALLNLNVIWM VIPLSNANQP EQVILYQGGQ MFDGALRFHG RVGFTGTMPA
              110 120 130 140 150
TNVSIFINNT QLSDTGTYQC LVNNLPDRGG RNIGVTGLTV LVPPSAPQCQ
              160 170 180 190 200
IQGSQDLGSD VILLCSSEEG IPRPTYLWEK LDNTLKLPPT ATQDQVQGTV
              210 220 230 240 250
TIRNISALSS GLYQCVASNA IGTSTCLLDL QVISPQPRSV GVIAGAVGTG
              260 270 280 290 300
AVLIVICLAL ISGAFFYWRS KNKEEEEEEI PNEIREDDLP PKCSSAKAFH
              310 320 330 340 350
TEISSSENNT LTSSNTYNSR YWNNNPKPHR NTESFNHFSD LRQSFSGNAV
              360 370 380 390 400
IPSIYANGNH LVLGPHKTLV VTANRGSSPQ VLPRNNGSVS RKPWPQHTHS
              410 420
YTVSQMTLER IGAVPVMVPA QSRAGSLV
SEQ ID NO. 379
(Human VSIR protein; UniProt identifier Q9H7M9):
                10 20 30 40 50
MGVPTALEAG SWRWGSLLFA LFLAASLGPV AAFKVATPYS LYVCPEGQNV
                60 70 80 90 100
TLTCRLLGPV DKGHDVTFYK TWYRSSRGEV QTCSERRPIR NLTFQDLHLH
              110 120 130 140 150
HGGHQAANTS HDLAQRHGLE SASDHHGNFS ITMRNLTLLD SGLYCCLVVE
              160 170 180 190 200
IRHHHSEHRV HGAMELQVQT GKDAPSNCVV YPSSSQDSEN ITAAALATGA
              210 220 230 240 250
CIVGILCLPL ILLLVYKQRQ AASNRRAQEL VRMDSNIQGI ENPGFEASPP
              260 270 280 290 300
AQGIPEAKVR HPLSYVAQRQ PSESGRHLLS EPSTPLSPPG PGDVFFPSLD
              310
PVPDSPNFEV I
SEQ ID NO. 380
(ECD of human VSIR protein; UniProt identifier Q9H7M9):
                10 20 30 40 50
FKVATPYSLY VCPEGQNVTL TCRLLGPVDK GHDVTFYKTW YRSSRGEVQT
                60 70 80 90 100
CSERRPIRNL TFQDLHLHHG GHQAANTSHD LAQRHGLESA SDHHGNFSIT
              110 120 130 140 150
MRNLTLLDSG LYCCLVVEIR HHHSEHRVHG AMELQVQTGK DAPSNCVVYP
              160
SSSQDSENIT AA
SEQ ID NO. 381
(Ig-like V-type domain of human VSIR protein; UniProt identifier Q9H7M9):
                10 20 30 40 50
FKVATPYSLY VCPEGQNVTL TCRLLGPVDK GHDVTFYKTW YRSSRGEVQT
                60 70 80 90 100
CSERRPIRNL TFQDLHLHHG GHQAANTSHD LAQRHGLESA SDHHGNFSIT
              110 120 130
MRNLTLLDSG LYCCLVVEIR HHHSEHRVHG AMELQV
SEQ ID NO. 382
(Rhesus monkey VSIR protein; UniProt identifier F7GVN3):
                10 20 30 40 50
MGVPTAPEAG CWRWGSLLFA LFLAASLGPV AAFKVATLYS LYVCPEGQNV
                60 70 80 90 100
TLTCRFFGPV DKGHDVTFYK TWYRSSRGEV QTCSERRPIR NLTFQDLHLH
              110 120 130 140 150
HGGHQAANTS HDLAQRHGLE SASDHHGNFS ITMRNLTLLD SGLYCCLVVE
              160 170 180 190 200
IRHHHSEHRV HGAMELQVQT GKDAPSSCVA YPSSSQESEN ITAAALATGA
              210 220 230 240 250
CIVGILCLPL ILLLVYKQRQ AASNRRDNTQ GIENPGFEAS SPAQGILEAK
              260 270 280 290 300
VRHPLSYVAQ RQPSESGRHL LSEPGTPLSP PGPGDVFFPS LDPVPDSPNF
EVI
SEQ ID NO. 383
(Murine VSIR protein; UniProt identifier Q9D659):
                10 20 30 40 50
MGVPAVPEAS SPRWGTLLLA IFLAASRGLV AAFKVTTPYS LYVCPEGQNA
                60 70 80 90 100
TLTCRILGPV SKGHDVTIYK TWYLSSRGEV QMCKEHRPIR NFTLQHLQHH
              110 120 130 140 150
GSHLKANASH DQPQKHGLEL ASDHHGNFSI TLRNVTPRDS GLYCCLVIEL
              160 170 180 190 200
KNHHPEQRFY GSMELQVQAG KGSGSTCMAS NEQDSDSITA AALATGACIV
              210 220 230 240 250
GILCLPLILL LVYKQRQVAS HRRAQELVRM DSNTQGIENP GFETTPPFQG
              260 270 280 290 300
MPEAKTRPPL SYVAQRQPSE SGRYLLSDPS TPLSPPGPGD VFFPSLDPVP
DSPNSEAI
SEQ ID NO. 384
(siRNA sequence targeting human IGSF11):
                10
CAACAUACCA UCCAUUUAU
SEQ ID NO. 385
(siRNA sequence targeting human IGSF11):
                10
GGAACGAAUU GGUGCAGUA
SEQ ID NO. 386
(siRNA sequence targeting human IGSF11):
                10
GAACAUCAGU GCCCUGUCU
SEQ ID NO. 387
(siRNA sequence targeting human IGSF11):
                10
CAGGAACAUU GGACUAAUA
SEQ ID NO. 388
(an IgC2 domain of human IGSF11 protein):
                10 20 30 40 50
GLTVLVPPSA PHCQIQGSQD IGSDVILLCS SEEGIPRPTY LWEKLDNTLK
                60 70 80 90 100
LPPTATQDQV QGTVTIRNIS ALSSGLYQCV ASNAIGTSTC LLDLQVISPQ
              110
PRNIG
SEQ ID NO. 389
(an IgV domain of human IGSF11 protein):
                10 20 30 40 50
LEVSESPGSI QVARGQPAVL PCTFTTSAAL INLNVIWMVT PLSNANQPEQ
                60 70 80 90 100
VILYQGGQMF DGAPRFHGRV GFTGTMPATN VSIFINNTQL SDTGTYQCLV
              110 120
NNLPDIGGRN IGVTGLTVLV P
SEQ ID NO. 390
(an IgC2 domain of human IGSF11 protein described by Wang et al, 2018):
                10 20 30 40 50
PSAPHCQIQG SQDIGSDVIL LCSSEEGIPR PTYLWEKLDN TLKLPPTATQ
                60 70 80 90
DQVQGTVTIR NISALSSGLY QCVASNAIGT STCLLDLQVI SPQPRNIG
```

SEQ ID NOs. 391 to 680 (Amino acid sequences of CDR and variable regions of ABPs of the invention, as well as nucleic acid sequences encoding variable regions of ABPs of the invention): See Table 13.1A.

SEQ ID NOs. 681 to 1070 (Amino acid sequences of CDR and variable regions of ABPs of the invention, as well as nucleic acid sequences encoding variable regions of ABPs of the invention): See Table 13.3.

1. A method for identifying, generating and/or producing an ABP that specifically binds to a C2-type immunoglobulin-like (IgC2) domain of IGSF11 (VSIG3) protein or a variant thereof, the method comprising the use of such IgC2 domain of IGSF11 (or variant or epitope thereof): (i) to screen a display library of a plurality of ABPs; or (ii) to immunise an animal, in particular a mammal,
wherein, the use comprises the use of a protein that comprises at least one epitope of (or comprised in) the IgC2 domain of IGSF11 (or variant thereof) and does not comprise an IgV domain of IGSF11 or a variant or epitope thereof; or
wherein, the use comprises the uses of a nucleic acid that encodes a protein that comprises at least one epitope of (or comprised in) the IgC2 domain of IGSF11 (or variant thereof) and does not encode a protein that comprises an IgV domain of IGSF11 or a variant or epitope thereof.

2. The method of claim 1, comprising the steps of:
X):
screening a display library, in particular a phage display library, that displays a plurality of ABPs with the protein; and
identifying an ABP that specifically binds to the IgC2 domain of IGSF11 or variant thereof, or
(Y):
administering to the animal an immunisation composition comprising the protein or the nucleic acid, and optionally together with a pharmaceutically acceptable carrier and/or excipient; and
isolating from the animal: (i) sera that comprises an ABP that specifically binds to the IgC2 domain of IGSF11 or variant thereof; and/or (ii) B cells that express an ABP that specifically binds the IgC2 domain of IGSF11 or variant thereof, and
further comprising the step of isolating, in particular purifying, an ABP that specifically binds to the IgC2 domain of IGSF11 or variant thereof.

3. A method for identifying and/or characterising an ABP as one specifically binding to a C2-type immunoglobulin-like (IgC2) domain of IGSF11 (VSIG3) protein or a variant thereof, the method comprising the step of:
detecting binding of the ABP to an epitope of (or comprised in) the IgC2 domain of IGSF11 protein (or variant thereof),
thereby identifying and/or characterising the ABP as one that specifically binds to the IgC2 domain of IGSF11 protein, or variant thereof.

4. The method of claim 3, further comprising the step of:
testing for binding of the ABP to an epitope of (or comprised in) an IgV domain of IGSF11 protein or, optionally, a variant thereof,
wherein, absence of detectable binding of the ABP to the epitope of (or comprised in) such IgV domain of IGSF11 protein (or variant thereof) further characterises the ABP as one that specifically binds to the IgC2 domain of IGSF11 protein, or variant thereof.

5. The method of claim 3 or 4, wherein:
the detecting step of claim 3 comprises detecting binding of the ABP to a first test protein, wherein the first test protein: (i) comprises the IgC2 domain of IGSF11 or a variant or fragment of such domain; and (ii) does not comprise the IgV domain of IGSF11 or, optionally, a variant thereof; and/or
the testing step of claim 4 comprises testing for binding of the ABP to a second test protein, wherein the second test protein: (a) comprises the IgV domain of IGSF11 or a variant or fragment of such domain; and (b) does not comprise the IgC2 domain of IGSF11 or a variant or fragment of such domain

6. The method of claim 5, wherein:
the first test protein does not comprise an IgV domain of IGSF11 or a variant or fragment of such domain; and/or
the second test protein comprises the IgV domain of IGSF11 or, optionally, a variant thereof.

7. The method of any one of claims claim 1 to 6, wherein the ABP that that specifically binds to the IgC2 domain of IGSF11 a variant thereof is, in particular further and/or thereby identified and/or characterised as, one for use in medicine.

8. The method of any one of claims claim 1 to 7, comprising the step of determining whether such ABP is able to enhance or increase killing and/or lysis of tumour cells, preferably cancer cells or cells; and in particular of whether such ABP is an anti-tumour ABP and/or is able to inhibit tumour growth in-vivo, preferably in a murine model of cancer.

9. The method of claim 8, wherein an ABP determined to have such (functional) characteristic (or characteristcs) is thereby determined as one that is for use in medicine.

10. The method of claim 9 further comprising the steps of producing (or having produced) an isolated ABP determined to have such (functional) characteristic (or characteristcs), and formulating (or having formulated) said ABP as a pharmaceutical composition.

11. An isolated antigen binding protein (ABP) which specifically binds to a C2-type immunoglobulin-like (IgC2) domain of IGSF11 (VSIG3) protein or a variant thereof, and wherein the isolated ABP comprises at least one complementarity determining region (CDR) and, optionally, is able to inhibit the binding of an interacting protein to IGSF11 protein or to an IgC2 domain of IGSF11 protein or, in either case, a variant thereof; optionally,
with the proviso that the ABP is not one or more of:
(A) one or more of an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown selected from any of the variable chain combinations Chains-A-001 to Chains-A-037 as described in Table C; and/or
(B) one or more of an antibody, or an antigen binding fragment thereof, composed of at least one, preferably two, antibody heavy chain sequence, and at least one, preferably two, antibody light chain sequence, wherein the antibody heavy chain sequence and the antibody light chain sequence each comprises a variable region sequence in a combination of heavy and light chain variable domain shown selected from any of the variable chain combinations Chains-B-001 to Chains-B-008 as described in Table C.1.

12. The isolated ABP of claim 11 comprising at least one CDR3 having an amino acid sequence with at least 90% sequence identity to, or having no more than three or two, preferably no more than one amino acid substitution(s), deletion(s) or insertion(s) compared to, a sequence selected from SEQ ID Nos.: 403, 407, 413, 417, 423, 427, 433, 437, 443, 447, 483, 487, 493, 497, 513, 517, 523, 527, 533, 537, 563, 567, 593, 597, 603, 607, 613 and 617.

13. The isolated ABP of claim 11 or 12 comprising at least one (heavy chain) complementarity determining region 3 (CDR3) having an amino acid sequence with at least 90% sequence identity to, or having no more than three or two, preferably no more than one amino acid substitution(s), deletion(s) or insertion(s) (in particular, substitution(s)) compared to a sequence selected from those (heavy chain) CDR3 sequences selected from any one sequence of the group consisting of SEQ ID NO: 403, 413, 423, 433, 443, 483, 493, 513, 523, 533, 563, 593, 603, and 613 (preferably compared to SEQ ID NO: 413 or 433).

14. The isolated ABP of any one of claims 11 to 13, further comprising at least one (heavy chain) CDR1 and at least one (heavy chain) CDR2, such as one from an antibody, in particular from a human antibody.

15. The isolated ABP of claim 14, wherein the least one (heavy chain) CDR1 and the at least one (heavy chain) CDR2, have an amino acid sequence with no more than five or four, such as having no more than three or two, preferably no more than one amino acid substitution(s), deletion(s) or insertion(s) (in particular, substitution(s)) compared to, a sequence selected from the corresponding (heavy chain) CDR1 and (heavy chain) CDR2 sequences shown in Table 13.1A or Table 13.3.

16. The isolated ABP of any one of claims 11 to 15, comprising an antibody heavy chain variable region CDR1, CDR2, and CDR3, and an antibody light chain variable region CDR1, CDR2, and CDR3.

17. The isolated ABP of any one of claims 11 to 16, which is an antibody or antigen binding fragment thereof.

18. The isolated ABP of any one of claims 11 to 17, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise CDR1 to CDR3 sequences in a combination selected from any of the following combinations of heavy and/or light chain CDRs: CDRs-C-002, CDRs-C-003, CDRs-C-004, CDRs-C-005, CDRs-C-006, CDRs-C-010, CDRs-C-011, CDRs-C-013, CDRs-C-014, CDRs-C-015, CDRs-C-018, CDRs-C-021, CDRs-C-022 and CDRs-C-023,

| | Heavy Chain | | | Light Chain | | |
|---|---|---|---|---|---|---|
| Combination | CDR1 to CDR3 | | | CDR1 to CDR3 | | |
| (ID) | (SEQ ID NO) | | | (SEQ ID NO) | | |
| CDRs-C-002 | 401 | 402 | 403 | 405 | 406 | 407 |
| CDRs-C-003 | 411 | 412 | 413 | 415 | 416 | 417 |
| CDRs-C-004 | 421 | 422 | 423 | 425 | 426 | 427 |
| CDRs-C-005 | 431 | 432 | 433 | 435 | 436 | 437 |
| CDRs-C-006 | 441 | 442 | 443 | 445 | 446 | 447 |
| CDRs-C-010 | 481 | 482 | 483 | 485 | 486 | 487 |
| CDRs-C-011 | 491 | 492 | 493 | 495 | 496 | 497 |
| CDRs-C-013 | 511 | 512 | 513 | 515 | 516 | 517 |
| CDRs-C-014 | 521 | 522 | 523 | 525 | 526 | 527 |
| CDRs-C-015 | 531 | 532 | 533 | 535 | 536 | 537 |
| CDRs-C-018 | 561 | 562 | 563 | 565 | 566 | 567 |
| CDRs-C-021 | 591 | 592 | 593 | 595 | 596 | 597 |
| CDRs-C-022 | 601 | 602 | 603 | 605 | 606 | 607 |
| CDRs-C-023 | 611 | 612 | 613 | 615 | 616 | 617 |

in each case independently, optionally with no more than three or two, preferably no more than one, amino acid substitution(s), insertion(s) or deletion(s) compared to these sequences.

19. The isolated ABP of any one of claims 11 to 18, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences each comprises heavy chain CDR1 to CDR3 sequences in the combination CDRs-C-003 or CDRs-C-004, or in the combination CDRs-C-005, and at least one, preferably both, of the antibody light chain sequences each comprises light chain CDR1 to CDR3 sequences in the combination, respectively, CDRs-C-003 or CDRs-C-004, or in the combination CDRs-C-005, in each case independently, optionally with no more than one amino acid substitution(s), insertion(s) or deletion(s) compared to these sequences, and preferably wherein the ABP is able to inhibit the binding of the interacting protein to IGSF11 protein or to the IgC2 domain of IGSF11 protein or, in either case, a variant thereof, with an IC50 of 50 nM or 10 nM or less.

20. The isolated ABP of any one of claims 11 to 17, wherein the ABP is an antibody, or an antigen binding fragment thereof, composed of at least one, preferably two, antibody heavy chain sequences, and at least one, preferably two, antibody light chain sequences, wherein
(A) the at least one, preferably two, antibody heavy chain sequences comprise (i) an antibody heavy chain CDR3 having not more than one amino acid substitution, insertion or deletion compared to a heavy chain CDR3 sequence selected from the group consisting of SEQ ID NO: 403, 413, 423, 433, 443, 483, 493, 513, 523, 533, 563, 593, 603, and 613 (preferably compared to SEQ ID NO: 413 or 433); and comprise (ii) an antibody heavy chain CDR1 having no more than five or four, amino acid substitution(s), deletion(s) or insertion(s) (in particular, substitution(s)) compared to, a sequence selected from SEQ ID NOs. 401, 411, 421, 431, 441, 481, 491, 511, 521, 531, 561, 591, 601, and 611 (preferably compared to SEQ ID NO: 411 or 431)); and comprise (iii) an antibody heavy chain CDR2 having no more than five or four amino acid substitution(s), deletion(s) or insertion(s) (in particular, substitution(s)) compared to, a sequence selected from SEQ ID NOs. 402, 412, 422, 432, 442, 482, 492, 512, 522, 532, 562, 592, 602, and 612 (preferably compared to SEQ ID NO: 412 or 432);
(B) the at least one, preferably two, antibody light chain sequences comprise (i) an antibody light chain CDR3 having no more than eight, seven, six, five or four, such as having no more than three or two, amino acid substitution(s), deletion(s) or insertion(s) compared to, the light chain CDR3 sequence selected from the group consisting of SEQ ID NO: 407, 417, 427, 437, 447, 487, 497, 517, 527, 537, 567, 597, 607, and 617 (preferably compared to SEQ ID NO: 417 or 437[); and comprise (ii) an antibody light chain CDR1 having no more than one amino acid substitution, deletion or insertion (in particular, substitution) compared to, a sequence selected from SEQ ID NOs. 405, 415, 425, 435, 445, 485, 495, 515, 525, 535, 565, 595, 605, and 615 (preferably compared to SEQ ID NO: 415 or 435)); and comprise (iii) an antibody light chain CDR2 having no more than one amino acid substitution, deletion or insertion (in particular, substitution) compared to, a sequence selected from SEQ ID NOs. 406, 416, 426, 436, 446, 486, 496, 516, 526, 536, 566, 596, 606, and 616 (preferably compared to SEQ ID NO: 416 or 436).

21. The isolated ABP of any one of claims 11 to 20, wherein the ABP is an antibody, or an antigen binding fragment thereof, composed of at least one, preferably two, antibody heavy chain sequences, and at least one, preferably two, antibody light chain sequences, wherein:
(X) the at least one, preferably two, antibody heavy chain sequence comprises a variable region sequence selected from the sequences according to SEQ ID NO: 414 or 434, and wherein the least one, preferably two, antibody light chain sequence comprises a light chain variable domain shown in Table C.2; in each case independently, optionally with no more than fifteen, fourteen, thirteen, twelve or eleven (eg, for variable light chain), or with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences; and/or
(Y) the at least one, preferably two, antibody light chain sequence comprises a variable region sequence selected from the sequences according to SEQ ID NO: 418 or 438; and wherein the least one, preferably two, antibody heavy chain sequence comprises a heavy chain variable domain shown in Table C.2; in each case independently, optionally with no more than fifteen, fourteen, thirteen, twelve or eleven (eg, for variable light chain), or with no more than about 20, 18, 16, 14 or 12, or no more than ten, nine, eight, seven, six, five, four, preferably no more than three, two or one, amino acid substitution(s), insertion(s) or deletion(s) (in particular, substitution(s)) compared to these sequences.

22. The isolated ABP of any one of claims 11 to 21, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences and at least one, preferably both, of the antibody light chain sequences comprise CDR1 to CDR3 sequences in a combination selected from any of the following combinations of heavy and/or light chain CDRs, CDRs-D-101 to CDRs-D-116 and CDRs-D-201 to CDRs-D-223:

| | Heavy Chain | | | Light Chain | | |
|---|---|---|---|---|---|---|
| Combination | CDR1 to CDR3 | | | CDR1 to CDR3 | | |
| (ID) | (SEQ ID NO) | | | (SEQ ID NO) | | |
| CDRs-D-101 | 681 | 682 | 683 | 685 | 686 | 687 |
| CDRs-D-102 | 691 | 692 | 693 | 695 | 696 | 697 |
| CDRs-D-103 | 701 | 702 | 703 | 705 | 706 | 707 |
| CDRs-D-104 | 711 | 712 | 713 | 715 | 716 | 717 |
| CDRs-D-105 | 721 | 722 | 723 | 725 | 726 | 727 |
| CDRs-D-106 | 731 | 732 | 733 | 735 | 736 | 737 |
| CDRs-D-107 | 741 | 742 | 743 | 745 | 746 | 747 |
| CDRs-D-108 | 751 | 752 | 753 | 755 | 756 | 757 |
| CDRs-D-109 | 761 | 762 | 763 | 765 | 766 | 767 |
| CDRs-D-110 | 771 | 772 | 773 | 775 | 776 | 777 |
| CDRs-D-111 | 781 | 782 | 783 | 785 | 786 | 787 |
| CDRs-D-112 | 791 | 792 | 793 | 795 | 796 | 797 |
| CDRs-D-113 | 801 | 802 | 803 | 805 | 806 | 807 |
| CDRs-D-114 | 811 | 812 | 813 | 815 | 816 | 817 |
| CDRs-D-115 | 821 | 822 | 823 | 825 | 826 | 827 |
| CDRs-D-116 | 831 | 832 | 833 | 835 | 836 | 837 |
| CDRs-D-201 | 841 | 842 | 843 | 845 | 846 | 847 |
| CDRs-D-202 | 851 | 852 | 853 | 855 | 856 | 857 |
| CDRs-D-203 | 861 | 862 | 863 | 865 | 866 | 867 |
| CDRs-D-204 | 871 | 872 | 873 | 875 | 876 | 877 |
| CDRs-D-205 | 881 | 882 | 883 | 885 | 886 | 887 |
| CDRs-D-206 | 891 | 892 | 893 | 895 | 896 | 897 |
| CDRs-D-207 | 901 | 902 | 903 | 905 | 906 | 907 |
| CDRs-D-208 | 911 | 912 | 913 | 915 | 916 | 917 |
| CDRs-D-209 | 921 | 922 | 923 | 925 | 926 | 927 |
| CDRs-D-210 | 931 | 932 | 933 | 935 | 936 | 937 |
| CDRs-D-211 | 941 | 942 | 943 | 945 | 946 | 947 |
| CDRs-D-212 | 951 | 952 | 953 | 955 | 956 | 957 |
| CDRs-D-213 | 961 | 962 | 963 | 965 | 966 | 967 |
| CDRs-D-214 | 971 | 972 | 973 | 975 | 976 | 977 |
| CDRs-D-215 | 981 | 982 | 983 | 985 | 986 | 987 |
| CDRs-D-216 | 991 | 992 | 993 | 995 | 996 | 997 |
| CDRs-D-217 | 1001 | 1002 | 1003 | 1005 | 1006 | 1007 |
| CDRs-D-218 | 1011 | 1012 | 1013 | 1015 | 1016 | 1017 |
| CDRs-D-219 | 1021 | 1022 | 1023 | 1025 | 1026 | 1027 |
| CDRs-D-220 | 1031 | 1032 | 1033 | 1035 | 1036 | 1037 |
| CDRs-D-221 | 1041 | 1042 | 1043 | 1045 | 1046 | 1047 |
| CDRs-D-222 | 1051 | 1052 | 1053 | 1055 | 1056 | 1057 |
| CDRs-D-223 | 1061 | 1062 | 1063 | 1065 | 1066 | 1067 |

in each case independently, optionally with no more than three or two, preferably no more than one, amino acid substitution(s), insertion(s) or deletion(s) compared to these sequences.

23. The isolated ABP of any one of claims 11 to 22, wherein the ABP comprises:
an antibody heavy chain sequence comprising a heavy chain variable domain sequence of SEQ ID Nos: 414, optionally with no more than ten, nine, eight, seven, six, five, four, preferably no more than three, two or one, amino acid substitution(s), insertion(s) or deletion(s) compared to this sequence, or an antigen binding fragment thereof, wherein the antibody heavy chain sequence or antigen binding fragment thereof comprises:
a CDR3 having the heavy chain CDR3 sequence SEQ ID No: 413, or having no more than one amino acid substitution(s), deletion(s) or insertion(s) compared to, the heavy chain CDR3 sequence SEQ ID No: 413;
a CDR1 having the heavy chain CDR1 sequence SEQ ID No: 411, or having no more than four or three, such as having no more than two or one, amino acid substitution(s), deletion(s) or insertion(s) compared to, the heavy chain CDR1 sequence SEQ ID No: 411; and
a CDR2 having the heavy chain CDR2 SEQ ID No: 412, or having no more than five or four, or three, such as having no more than three or two, or having no more than one, amino acid substitution(s), deletion(s) or insertion(s) compared to, the heavy chain CDR2 SEQ ID No: 412, and
an antibody light chain sequence comprising a light chain variable domain sequence of SEQ ID Nos: 418, optionally with no more than ten, nine, eight, seven, six, five, four, preferably no more than three, two or one, amino acid substitution(s), insertion(s) or deletion(s) compared to this sequence, or an antigen binding fragment thereof, wherein the antibody light chain sequence or antigen binding fragment thereof comprises:
a CDR3 having the light chain CDR3 sequence SEQ ID No: 417, or having no more than nine, eight, seven, six, five or four, such as having no more than three or two, or having no more than one, amino acid substitution(s), deletion(s) or insertion(s) compared to, the light chain CDR3 sequence SEQ ID No: 417;
a CDR1 having the light chain CDR1 sequence SEQ ID No: 415, or having no more than one amino acid substitution(s), deletion(s) or insertion(s) compared to, the light chain CDR1 sequence SEQ ID No: 415; and
a CDR2 having the light chain CDR2 sequence SEQ ID No: 416, or having no more than one amino acid substitution(s), deletion(s) or insertion(s) compared to, the light chain CDR2 sequence SEQ ID No: 416.

24. The isolated ABP of any one of claims 11 to 22, wherein the ABP comprises:
an antibody heavy chain sequence comprising a heavy chain variable domain sequence of SEQ ID Nos: 434, optionally with no more than ten, nine, eight, seven, six, five, four, preferably no more than three, two or one, amino acid substitution(s), insertion(s) or deletion(s) compared to this sequence, or an antigen binding fragment thereof, wherein the antibody heavy chain sequence or antigen binding fragment thereof comprises:
a CDR3 having the heavy chain CDR3 sequence SEQ ID No: 433, or having no more than one amino acid substitution(s), deletion(s) or insertion(s) compared to, the heavy chain CDR3 sequence SEQ ID No: 433;
a CDR1 having the heavy chain CDR1 sequence SEQ ID No: 431, or having no more than three or two, or having no more than one, amino acid substitution(s), deletion(s) or insertion(s) compared to, the heavy chain CDR1 sequence SEQ ID No: 431; and
a CDR2 having the heavy chain CDR2 SEQ ID No: 432, or having no more than nine, eight, seven, six, five or four, such as having no more than three or two, or having no more than one, amino acid substitution(s), deletion(s) or insertion(s) compared to, the heavy chain CDR2 SEQ ID No: 432, and
an antibody light chain sequence comprising a light chain variable domain sequence of SEQ ID Nos: 438, optionally with no more than ten, nine, eight, seven, six, five, four, preferably no more than three, two or one, amino acid substitution(s), insertion(s) or deletion(s) compared to this sequence, or an antigen binding fragment thereof, wherein the antibody light chain sequence or antigen binding fragment thereof comprises:
a CDR3 having the light chain CDR3 sequence SEQ ID No: 437, or having no more than six, or five or four, such as having no more than three or two, or having no more than one, amino acid substitution(s), deletion(s) or insertion(s) compared to, the light chain CDR3 sequence SEQ ID No: 437;
a CDR1 having the light chain CDR1 sequence SEQ ID No: 435, or having no more than one amino acid substitution(s), deletion(s) or insertion(s) compared to, the light chain CDR1 sequence SEQ ID No: 435; and
a CDR2 having the light chain CDR2 sequence SEQ ID No: 436, or having no more than one amino acid substitution(s), deletion(s) or insertion(s) compared to, the light chain CDR2 sequence SEQ ID No: 436.

25. The isolated ABP of any one of claims 1 to 24, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences each comprises heavy chain CDR1 to CDR3 sequences in the combination CDRs-D-114 or CDRs-D-222, in each case independently, optionally with no more than one amino acid substitution(s), insertion(s) or deletion(s) compared to these sequences, and preferably wherein the ABP is able to inhibit the binding of the interacting protein to IGSF11 protein or to the IgC2 domain of IGSF11 protein or, in either case, a variant thereof, with an IC50 of 50 nM or 10 nM, or 0.5 nM or less, preferably as measured according to example 13 herein.

26. The isolated ABP of any one of claims 11 to 25, wherein the ABP is an antibody, or an antigen binding fragment thereof, composed of at least one, preferably two, antibody heavy chain sequences, and at least one, preferably two, antibody light chain sequences, wherein at least one, preferably both, of the antibody light chain sequences each comprises heavy chain CDR1 to CDR3 sequences in the combination CDRs-D-114 or CDRs-D-222, in each case independently, optionally with no more than one amino acid substitution(s), insertion(s) or deletion(s) compared to these sequences, and preferably wherein the ABP is able to inhibit the binding of the interacting protein to IGSF11 protein or to the IgC2 domain of IGSF11 protein or, in either case, a variant thereof, with an IC50 of 50 nM or 10 nM, or 0.5 nM or less, preferably as measured according to example 13 herein.

27. The isolated ABP of any one of claims 11 to 28, wherein the ABP is an antibody, or an antigen binding fragment thereof, composed of at least one, preferably two, antibody heavy chain sequences, and at least one, preferably two, antibody light chain sequences, wherein at least one, preferably both, of the antibody heavy chain sequences each comprises heavy chain CDR1 to CDR3 sequences in the combination CDRs-D-114 or CDRs-D-222, and at least one, preferably both, of the antibody light chain sequences each comprises light chain CDR1 to CDR3 sequences in the combination, respectively, CDRs-D-114 or CDRs-D-222, in each case independently, optionally with no more than one amino acid substitution(s), insertion(s) or deletion(s) compared to these sequences, and preferably wherein the ABP is able to inhibit the binding of the interacting protein to IGSF11 protein or to the IgV domain of IGSF11 protein or, in either case, a variant thereof, with an IC50 of 50 nM or 10 nM, or 0.5 nM or less, preferably as measured according to example 13 herein.

28. The isolated ABP of any one of claims 11 to 27, wherein the ABP comprises:
an antibody heavy chain sequence comprising a heavy chain variable domain sequence of SEQ ID Nos: 814, optionally with no more than ten, nine, eight, seven, six, five, four, preferably no more than three, two or one, amino acid substitution(s), insertion(s) or deletion(s) compared to this sequence, or an antigen binding fragment thereof, wherein the antibody heavy chain sequence or antigen binding fragment thereof comprises:
a CDR3 having the heavy chain CDR3 sequence SEQ ID No: 813, or having no more than one amino acid substitution(s), deletion(s) or insertion(s) compared to, and preferably having no amino acid substitution(s), insertion(s) or deletion(s) compared to, the heavy chain CDR3 sequence SEQ ID No: 813;
a CDR1 having the heavy chain CDR1 sequence SEQ ID No: 811, or having no more than four, three or two, or having no more than one, amino acid substitution(s), deletion(s) or insertion(s) compared to, and preferably having no amino acid substitution(s), insertion(s) or deletion(s) compared to, the heavy chain CDR1 sequence SEQ ID No: 811; and
a CDR2 having the heavy chain CDR2 SEQ ID No: 812, or having no more than five or four, such as having no more than three or two, or having no more than one, amino acid substitution(s), deletion(s) or insertion(s) compared to, and preferably having no amino acid substitution(s), insertion(s) or deletion(s) compared to, the heavy chain CDR2 SEQ ID No: 812, and
an antibody light chain sequence comprising a light chain variable domain sequence of SEQ ID Nos: 818, optionally with no more than ten, nine, eight, seven, six, five, four, preferably no more than three, two or one, amino acid substitution(s), insertion(s) or deletion(s) compared to this sequence, or an antigen binding fragment thereof, wherein the antibody light chain sequence or antigen binding fragment thereof comprises:
a CDR3 having the light chain CDR3 sequence SEQ ID No: 817, or having no more than nine, eight, seven, six, or five, such as having no more than four, three or two, or having no more than one, amino acid substitution(s), deletion(s) or insertion(s) compared to, and preferably having no amino acid substitution(s), insertion(s) or deletion(s) compared to, the light chain CDR3 sequence SEQ ID No: 817;
a CDR1 having the light chain CDR1 sequence SEQ ID No: 815, or having no more than one amino acid substitution(s), deletion(s) or insertion(s) compared to, and preferably having no amino acid substitution(s), insertion(s) or deletion(s) compared to, the light chain CDR1 sequence SEQ ID No: 815; and
a CDR2 having the light chain CDR2 sequence SEQ ID No: 816, or having no more than one amino acid substitution(s), deletion(s) or insertion(s) compared to, and preferably having no amino acid substitution(s), insertion(s) or deletion(s) compared to, the light chain CDR2 sequence SEQ ID No: 816.

29. The isolated ABP of any one of claims 11 to 27, wherein the ABP comprises:
an antibody heavy chain sequence comprising a heavy chain variable domain sequence of SEQ ID Nos: 1054, optionally with no more than ten, nine, eight, seven, six, five, four, preferably no more than three, two or one, amino acid substitution(s), insertion(s) or deletion(s) compared to this sequence, or an antigen binding fragment thereof, wherein the antibody heavy chain sequence or antigen binding fragment thereof comprises:
a CDR3 having the heavy chain CDR3 sequence SEQ ID No: 1053, or having no more than one amino acid substitution(s), deletion(s) or insertion(s) compared to, and preferably having no amino acid substitution(s), insertion(s) or deletion(s) compared to, the heavy chain CDR3 sequence SEQ ID No: 1053;
a CDR1 having the heavy chain CDR1 sequence SEQ ID No: 1051, or having no more than three or two, or having no more than one, amino acid substitution(s), deletion(s) or insertion(s) compared to, and preferably having no amino acid substitution(s), insertion(s) or deletion(s) compared to, the heavy chain CDR1 sequence SEQ ID No: 1051; and
a CDR2 having the heavy chain CDR2 SEQ ID No: 1052, or having no more than nine, eight, seven, six, five, four, preferably no more than three, two or one, amino acid substitution(s), deletion(s) or insertion(s) compared to, and preferably having no amino acid substitution(s), insertion(s) or deletion(s) compared to, the heavy chain CDR2 SEQ ID No: 1052, and
an antibody light chain sequence comprising a light chain variable domain sequence of SEQ ID Nos: 1058, optionally with no more than ten, nine, eight, seven, six, five, four, preferably no more than three, two or one, amino acid substitution(s), insertion(s) or deletion(s) compared to this sequence, or an antigen binding fragment thereof, wherein the antibody light chain sequence or antigen binding fragment thereof comprises:
a CDR3 having the light chain CDR3 sequence SEQ ID No: 1057, or having no more than six, five or four, such as having no more than three or two, or having no more than one, amino acid substitution(s), deletion(s) or insertion(s) compared to, and preferably having no amino acid substitution(s), insertion(s) or deletion(s) compared to, the light chain CDR3 sequence SEQ ID No: 1057;
a CDR1 having the light chain CDR1 sequence SEQ ID No: 1055, or having no more than one amino acid substitution(s), deletion(s) or insertion(s) compared to, and preferably having no amino acid substitution(s), insertion(s) or deletion(s) compared to, the light chain CDR1 sequence SEQ ID No: 1055; and
a CDR2 having the light chain CDR2 sequence SEQ ID No: 1056, or having no more than one amino acid substitution(s), deletion(s) or insertion(s) compared to, and preferably having no amino acid substitution(s), insertion(s) or deletion(s) compared to, the light chain CDR2 sequence SEQ ID No: 1056.

30. The isolated ABP according to any one of claims 1 to 29, that binds to an IgC2 domain of IGSF11 with a KD that is less than about 1 nM, preferably less than 150 pM or less than 100 pM, even more preferably with a KD that is less than 10 pM; and optionally as measured according to example 14 herein, such as using a kinetic exclusion assay.

31. An isolated ABP which competes with an ABP as recited in any one of claims 11 to 30 for binding to an IgC2 domain of IGSF11 protein or a variant thereof, and, optionally, is able to inhibit the binding of an interacting protein to IGSF11 protein or to an IgC2 domain of IGSF11 protein or, in each case, a variant thereof,
with the proviso that the isolated ABP is not one or more of:
any ABP the subject of proviso (A) of claim 11;
any ABP the subject of proviso (B) of claim 11.

32. The isolated ABP of any one of claims 11 to 31, wherein the interacting protein is VSIR (VISTA) protein or a variant thereof.

33. The isolated ABP of any one of claims 11 to 32 that is able to enhance or increase killing and/or lysis of cells expressing IGSF11 or an IgC2 domain of IGSF11, or a variant thereof.

34. The isolated ABP of any one of claims 11 to 33 that is able to enhance or increase killing and/or lysis of tumour cells, preferably cancer cell or cells that originate from a tumour cell and/or cells that express IGSF11 or an IgC2 domain of IGSF11, or a variant thereof.

35. The isolated ABP of any one of claims 11 to 34 that is an anti-tumour ABP.

36. The isolated ABP of any one of claims 11 to 35 that is able to inhibit tumour growth in-vivo, preferably in a murine model of cancer.

37. The isolated ABP of any one of claims 11 to 36 that enhances killing and/or lysis of cells expressing IGSF11, or a variant of IGSF11, by cytotoxic T cells and/or TILs.

38. The isolated ABP of any one of claims 11 to 37 that (i) enhances a cell-mediated immune response, such as that mediated by an activated cytotoxic T-cell (CTL), to a mammalian cell expressing said IGSF11 or the variant of IGSF11; and/or (ii) increases immune cell, such as T-cell, activity and/or survival in the presence of a mammalian cell expressing said IGSF11 or the variant of IGSF11.

39. The isolated ABP of any one of claims 11 to 38 that modifies the microenvironment of a tumour, in particular modulates the number and/or type of immune cells present in the tumour, and more suitably reduces the number of intra-tumoural myeloid-derived suppressor cells (MDSCs) and/or increases the number of intra-tumoural CTLs.

40. The isolated ABP of any one of claims 11 to 39 that decreases (the number of M2) tumour-associated macrophages (TAMs) and/or increases the number of (intra-tumoural) CTLs, optionally, in each case, within the tumour microenvironment.

41. The isolated ABP of any one of claims 11 to 40, wherein the ABP is able to inhibit the binding of an interacting protein to IGSF11 protein or to an IgC domain or of IGSF11 protein or, in either case, a variant thereof; optionally with an IC50 of 50 nM or 10 nM or less, or preferably 0.5 nM or less.

42. The isolated ABP of any one of claims 11 to 41, wherein the ABP does not inhibit the interaction between VSIR (VISTA) protein or a variant thereof and IGSF11 protein or the IgC2 domain of IGSF11 protein or a variant thereof.

43. The isolated ABP of any one of claims 11 to 42 that is an antibody or an antigen binding fragment thereof, wherein the antibody is a monoclonal antibody, or wherein the antigen binding fragment is a fragment of a monoclonal antibody.

44. The isolated ABP of any one of claims 11 to 43 that is multi-specific, in particular is bi-specific (such as a bispecific T-cell engager (BiTE) ABP or antibody).

45. The isolated ABP of any one of claims 11 to 44 that is a chimeric antigen receptor (CAR).

46. An isolated nucleic acid encoding for an ABP, or for an antigen binding fragment or a monomer of an ABP, wherein the ABP is one of any one of claims 11 to 45.

47. A recombinant host cell comprising a nucleic acid recited in claim 46.

48. A pharmaceutical composition comprising:
(X):
(i) an ABP of any one of claims 11 to 45; or
(ii) a nucleic acid recited in claim 46 or a recombinant host cell of claim 47, in particular a T cell comprising a nucleic acid expressing an ABP comprising a chimeric antigen receptor (CAR); or
(iii) a compound that is an inhibitor of the expression, function, activity and/or stability of immunoglobulin superfamily member 11 (IGSF11, or VSIG3), or of a C2-type immunoglobulin-like (IgC2) domain of IGSF11 or of a variant thereof,
with the proviso that the compound is not one or more of:
any ABP the subject of proviso (A) of claim 11;
any ABP the subject of proviso (B) of claim 11;
(Y):
a pharmaceutically acceptable carrier, stabiliser and/or excipient.

49. A product for use in medicine, wherein the product is selected from the list consisting of:
(i) an isolated ABP of any one of claims 11 to 45, and
(ii) an isolated nucleic acid recited in claim 46 or a recombinant host cell of claim 47, in particular T cell comprising a nucleic acid expressing an ABP comprising a chimeric antigen receptor (CAR), and
(iii) a compound that is an inhibitor of the expression, function, activity and/or stability of immunoglobulin superfamily member 11 (IGSF11, or VSIG3), or of a C2-type immunoglobulin-like (IgC2) domain of IGSF11) or of a variant thereof,
with the proviso that the compound is not one or more of:
any ABP the subject of proviso (A) of claim 11;
any ABP the subject of proviso (B) of claim 11;

50. The product for use in medicine of claim 49 wherein the product is for use in the treatment of a proliferative disorder that is associated with the undesired presence of IGSF11-positive cells or cells positive for a variant of IGSF11 and/or that is associated with cellular resistance against a cell-mediated immune response and/or that is associated with expression or activity of IGSF11 or a variant thereof of IGSF11.

51. The product for use in medicine of claim 50, wherein cells involved in the proliferative disorder are resistant to a cell-mediated immune response.

52. The product for use in medicine of any one of claims 49 to 51, wherein the product is for use in enhancing an immune response in a mammalian subject, preferably for use in aiding a cell-mediated immune response in the subject such as the subject's T cell mediated immune response, for example for treating a proliferative disease, such as a cancer disease, or for treating an infectious disease.

53. The product for use in medicine of any one of claims 49 to 52, wherein the product is for use in the treatment of a proliferative disorder resistant and/or refractory to PD1/PDL1 blockade therapy and/or to CTLA4 blockade therapy.

54. The product for use in medicine of any one of claims 49 to 53, wherein the product is for use in the treatment of a proliferative disorder in combination with a different anti-proliferative therapy.

55. The product for use in medicine of any one of claims 49 to 54, wherein the product is for use in the treatment of a cancer in combination with immunotherapy with a ligand to an immune checkpoint molecule, preferably the ligand is one that binds to an immune checkpoint molecule selected from the group consisting of: A2AR, B7-H3, B7-H4, CTLA-4, IDO, KIR, LAG3, PD-1 (or one of its ligands PD-L1 and PD-L2), TIM-3 (or its ligand galectin-9), TIGIT and VISTA.

56. The product for use in medicine of claim 55, wherein the ligand binds to an immune checkpoint molecule selected from CTLA-4, PD-1 and PD-L1.

57. An in-vitro method for determining whether a subject has, or is at risk of, developing a disease, disorder or condition that is associated with the undesired presence of IGSF11-positive cells (or cells positive for a variant of IGSF11) and/or that is associated with cellular resistance against a cell-mediated immune response and/or that is associated with expression or activity of IGSF11 (or a variant thereof), the method comprising the step of:
detecting a C2-type immunoglobulin-like (IgC2) domain of IGSF11 (or a variant of such domain), in particular the presence (or an amount) of or expression and/or activity of such domain of IGSF11 (or the variant thereof), in a biological sample from said subject,
wherein the detection of such domain of IGSF11 (or the variant thereof) in the sample indicates such disease, disorder or condition, or a risk of developing such disease, disorder or condition, in the subject; and
optionally, wherein such domain of the IGSF11 (or variant thereof) is detected with an ABP of any one of claims 11 to 45.

58. An in-vitro method for determining whether a subject has, or has a risk of developing, a disease, disorder or condition that is associated with the undesired presence of IGSF11-positive cells (or cells positive for a variant of IGSF11) and/or that is associated with cellular resistance against a cell-mediated immune response and/or that is associated with expression or activity of IGSF11 (or a variant thereof), the method comprising the steps of:
contacting cells of the subject involved with the disease, disorder or condition with an ABP of any one of claims 11 to 45, and/or with a product recited in any one of claims 49 to 56, in the presence of a cell-mediated immune response, preferably wherein the cell-mediated immune response comprises immune cells selected from the group consisting of: lymphocytes, T-cells, CTLs and TILs; and
determining the cell-mediated immune response against such cells of the subject,
wherein an enhancement of the cell-mediated immune response against such cells of the subject indicates that the subject has or has a risk of developing a disease, disorder or condition that is selected from a proliferative disorder or an infectious disease.

59. An in-vitro method for identifying and/or characterising a compound suitable for the treatment of a disease, disorder or condition that is associated with the undesired presence of IGSF11-positive cells (or cells positive for a variant of IGSF11) and/or that is characterised by cellular resistance against a cell-mediated immune response and/or one that is characterised by expression or activity of IGSF11 (or a variant thereof), the method comprising the steps of:
(a) bringing into contact a first cell expressing a protein comprising a C2-type immunoglobulin-like (IgC2) domain of IGSF11 (or a variant of such domain) and (x) the candidate compound, or (y) the candidate compound and a cell-mediated immune response, preferably wherein the cell-mediated immune response comprises immune cells selected from the group consisting of: lymphocytes, T-cells, CTLs and TILs; and
(b) determining (i) the expression, activity, function and/or stability of the (eg protein or mRNA of) such domain of IGSF11 (or variant), in the first cell; and/or (ii) the cell-mediated immune response against the first cell,
wherein: (i) a reduced expression, activity function and/or stability of such domain of IGSF11 (or variant), in said first cell contacted with the candidate compound compared to said first cell not contacted with said candidate compound; and/or (ii) an enhancement of the cell-mediated immune response against the first cell contacted with the candidate compound compared to the cell-mediated immune response against the first cell not contacted with the candidate compound; indicates that the candidate compound is a compound suitable for the treatment of a disease, disorder or condition that is selected from a proliferative disorder or an infectious disease; and
optionally, wherein the reduction of expression, activity function and/or stability of such domain of IGSF11 (eg, induction of internalisation of IGSF11 protein or such domain of IGSF11 protein) and/or the enhancement of the cell-mediated immune response is identified by reference to a control method practised with a compound having a known effect on such expression, function, activity and/or stability, in particular a positive or negative control; and wherein the compound having a known effect on such expression, function, activity and/or stability is an ABP of any one of claims 11 to 45 and/or is a product recited in any one of claims 49 to 56.

60. The method of claim 59, wherein the protein expressed by the first cell does not comprise the IgV domain of IGSF11.

61. A method for identifying and/or characterising an ABP as one specifically binding to a C2-type immunoglobulin-like (IgC2) domain of IGSF11 (VSIG3) protein or a variant thereof, the method comprising the step of:
detecting binding of the ABP to an epitope of (or comprised in) such domain of IGSF11 protein (or variant thereof),
thereby identifying and/or characterising the ABP as one that specifically binds to the IgC2 domain of IGSF11 protein, or variant thereof.

62. A method for identifying and/or characterising an ABP for use in medicine, the method comprising the steps of:
providing an ABP that binds to IGSF11 protein (or a variant thereof); and
identifying and/or characterising the provided ABP as one that specifically binds to an IgC2 domain of IGSF11 protein or a variant thereof,
thereby identifying and/or characterising the ABP for use in medicine.

63. A method for producing an ABP for use in medicine, the method comprising the steps of:
providing a hybridoma or (host) cell capable of expressing an ABP that binds to IGSF11 protein (or a variant thereof), for example a recombinant cell line comprising at least one genetic construct comprising coding sequence(s) encoding said ABP; and
culturing said hybridoma or host cell under conditions that allow for the expression of the ABP;
optionally, isolating the ABP expressed by said hybridoma or host cell; and
identifying and/or characterising the expressed ABP as one that specifically binds to an IgC2 domain of IGSF11 protein or a variant thereof,
thereby producing the ABP for use in medicine.

64. A use of an IgC2 domain of IGSF11 protein or a variant or fragment (eg, at least one epitope) of such domain to identify, characterise and/or produce an ABP for use in medicine, suitably wherein the ABP specifically binds to such domain of IGSF11 protein (or variant thereof).

65. The use of claim 64, further comprising the use of an IgV domain of IGSF11 protein or, optionally, a variant thereof, suitably wherein the ABP does not bind to such domain of IGSF11 protein (or variant thereof).

66. The use of claim 64 or 65, wherein the use comprises the use of:
a first test protein, wherein the test protein: (i) comprises the IgC2 domain of IGSF11 or a variant or fragment of such domain; and (ii) does not comprise an IgV domain of IGSF11 or, optionally, a variant thereof; and/or
a second test protein, wherein the second test protein: (a) comprises an IgV domain of IGSF11 or a variant or fragment of such domain thereof; and (b) does not comprise the IgC2 domain of IGSF11, or a fragment of such domain or, optionally, a variant thereof.

67. The use of claim 66, wherein:
the first test protein does not comprise an IgV domain of IGSF11 or a variant or fragment of such domain; and/or
the second test protein comprises the IgV domain of IGSF11 or a variant thereof.

68. The method of any one of claim 62 or 63, or the use of any one of claims 64 to 67, wherein the ABP for use in medicine is:
an ABP for use in the treatment of a proliferative disorder that is associated with the undesired presence of IGSF11-positive cells or cells positive for a variant of IGSF11 and/or that is associated with cellular resistance against a cell-mediated immune response and/or that is associated with expression or activity of IGSF11 or a variant thereof of IGSF11, suitable wherein cells involved in the proliferative disorder are resistant to a cell-mediated immune response;
an ABP for use in enhancing an immune response in a mammalian subject, preferably for use in aiding a cell-mediated immune response in a subject such as the subject's T cell mediated immune response, for example for treating a proliferative disease, such as a cancer disease, of for treating an infectious disease; and/or
an ABP for use in the treatment of a proliferative disorder resistant and/or refractory to PD1/PDL1 and/or CTLA4 blockade therapy.

69. The method of any one of claims 62, 63 or 68, or the use of any one of claims 64 to 67, wherein the ABP:
is capable of enhancing or increasing killing and/or lysis of cells expressing IGSF11 or an IgC2 domain (or IgV domain) of IGSF11, or a variant thereof;
is capable of enhancing or increasing killing and/or lysis of tumour cells, preferably cancer cell or cells that originate from a tumour cell and/or cells that express IGSF11 or an IgC2 domain (or IgV domain) of IGSF11, or a variant thereof;
is a therapeutic antibody able to treat, ameliorate and/or delay progression of a disease, disorder or condition, in particular a disease, disorder or condition mentioned herein elsewhere;
is an anti-tumour antibody;
is capable of inhibiting tumour growth in-vivo, preferably in a murine model of cancer;
is able to inhibit the binding of an interacting protein to IGSF11 protein or a variant thereof, suitably: (i) wherein the interacting protein is VSIR (VISTA) protein or a variant thereof; or, alternatively (ii) wherein the interacting protein is not VSIR (VISTA) protein or a variant thereof;
is able to inhibit (eg, inhibits) the interaction between VSIR (VISTA) protein or a variant thereof and the IgC2 domain (or the IgV domain) of IGSF11 protein or a variant thereof or, alternatively (ii) is not able to inhibit (eg, does not inhibit) the interaction between VSIR (VISTA) protein or a variant thereof and the IgC2 domain (or the IgV domain) of IGSF11 protein or a variant thereof;
enhances killing and/or lysis of cells expressing IGSF11, or a variant of IGSF11, by cytotoxic T cells and/or TIL;
enhances a cell-mediated immune response, such as that mediated by an activated cytotoxic T-cell (CTL), to a mammalian cell expressing said IGSF11 or the variant of IGSF11;
increases immune cell, such as T-cell, activity and/or survival in the presence of a mammalian cell expressing said IGSF11 or the variant of IGSF11;
modifies the microenvironment of a tumour, suitably increases the number and/or type of immune cells present in the tumour, and more suitably reduces the number of intra-tumoural MDSCs and/or increases the number of intra-tumoural CTLs;
recruits and/or activates NK cells and/or mediates antibody-dependent cellular cytotoxicity (ADCC);
recruits and/or activates macrophages and/or mediates antibody-dependent cellular phagocytosis (ADCP);
recruits complement and/or mediates complement dependent cytotoxicity (CDC); and/or
decreases (the number of) M2 tumour-associated macrophages (TAMs) and/or increases the number of (intra-tumoural) CTLs, optionally, in each case, within the tumour microenvironment: and/or
induces internalisation of IGSF11 protein from the surface of cells (such as tumour cells that express IGSF11).

70. The method of any one of claims 62, 63, 68 or 69, or the use of any one of claims 64 to 69, further comprising the step of: determining or having determined, that the ABP has one or more of the functional characteristics as set forth in any one of claims 30 to 42 or claim 69.

71. The method of any one of claims 62, 63, 68 to 70, or the use of any one of claims 64 to 70, wherein the ABP is an antibody, or an antigen binding fragment thereof, such as a monoclonal antibody, or wherein the antigen binding fragment is a fragment of a monoclonal antibody.

72. The method or use of claim 71, wherein the antibody is a human antibody a humanised antibody or a chimeric-human antibody, or wherein the antigen binding fragment is a fragment of a human antibody a humanised antibody or a chimeric-human antibody.

73. A method for treating a subject in need thereof, said treatment comprising inhibiting the interaction between IGSF11 protein and an interacting protein of IGSF11 protein, such as an interacting protein that binds to an IgC2 domain of the IGSF11 protein, the method comprising the step of:
administering to the subject a (eg, therapeutically effective amount of a) compound that is an inhibitor of the expression, function, activity and/or stability of an IgC2 domain of IGSF11 protein or a variant thereof,
with the proviso that the compound is not one or more of:
any ABP the subject of proviso (A) of claim 11;
any ABP the subject of proviso (B) of claim 11;
to inhibit the interaction between IGSF11 protein and an interacting protein of IGSF11 protein.

74. The method of claim 73, wherein the compound is an ABP of any one of claims 11 to 45.

75. The method of claim 73 or 74, wherein the interacting protein of IGSF11 protein is an endogenous binding partner of IGSF11 protein, and preferably is VSIR (VISTA) protein or a variant thereof.

76. A method for identifying, generating and/or producing an ABP that specifically binds to an IgC2 domain of IGSF11 or a variant thereof, the method comprising the use of such domain or an epitope of (or comprised in) such domain: (i) to screen a display library of a plurality of ABPs; or (ii) to immunise an animal.

77. The method of claim 76, wherein the use comprises the use of a protein that comprises at least one epitope of (or comprised in) the IgC2 domain of IGSF11 (or variant thereof), wherein the protein does not comprise an IgV domain of IGSF11 (or a variant or epitope thereof).

78. The method of claim 77, wherein the use comprises the use of a nucleic acid that encodes a protein comprising at least one epitope of (or comprised in) the IgC2 domain of IGSF11 (or variant thereof), wherein the nucleic acid does not encode a protein comprising an IgV domain of IGSF11 (or a variant or epitope thereof thereof).

79. The method of claim 78, comprising the step of immunising an animal (in particular a mammal, eg, a mouse, rat, rabbit, goat, camel, or llama) with a protein recited in claim 77 or with the nucleic acid recited in claim 78.

80. The method of claim 76, comprising a step of administering to the animal an immunisation composition comprising a protein recited in claim 77 or a nucleic acid recited in claim 78, and optionally together with a pharmaceutically acceptable carrier and/or excipient.

81. The method of claim 76, further comprising the step of isolating from the animal: (i) sera that comprises an ABP that specifically binds to said domain of IGSF11 (or variant thereof); and/or (ii) B cells that express an ABP that specifically binds to said domain of IGSF11 (or variant thereof).

82. The method of claim 76, comprising the steps of screening a display library (eg, a phage display library) that displays a plurality of ABPs with a protein of claim 77, and identifying an ABP that specifically binds to the said domain of IGSF11 (or variant thereof).

83. The method of claim 81 or 82, further comprising the step of isolating (eg, purifying) the ABP that specifically binds to the said domain of IGSF11 (or variant thereof).

84. The method of any one of claims 76 to 83, for identifying, generating and/or producing an ABP for use in medicine.

85. The method of claim 834 further comprising the step of: determining or having determined, that the ABP has one or more of the functional characteristics as set forth in any one of claims 30 to 42 or claim 69; optionally, wherein an ABP determined to have one or more of such functional characteristics is for use in medicine.


SEQUENCE LISTING (1070 sequences)

SEQ ID NO: 1 - protein, 5 aa, Homo sapiens
SYSMN

SEQ ID NO: 2 - protein, 17 aa, Homo sapiens
SISSSSSYIYYADSVKG

SEQ ID NO: 3 - protein, 15 aa, Homo sapiens
SIIVQALGITSVFDI

SEQ ID NO: 4 - protein, 124 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCVRSIIVQALGITSVFDIWGQGTMVTVSS

SEQ ID NO: 5 - protein, 11 aa, Homo sapiens
RASQIISNHLN

SEQ ID NO: 6 - protein, 7 aa, Homo sapiens
AASRLQT

SEQ ID NO: 7 - protein, 9 aa, Homo sapiens
QQSYSNPRT

SEQ ID NO: 8 - protein, 107 aa, Homo sapiens
AIRLTQSPSSLSASVGDRVTISCRASQIISNHLNWYQQKPGKAPKLLIYAASRLQTGVPSRFSGSGSETDYTLTISSLQPEDFATYYCQQSYSNPRTFGHGTKVEIK

SEQ ID NO: 9 - DNA, 372 bp, Homo sapiens
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtatattactgtgtcaggtctattatagttcaagcacttggcataacgtccgtttttgatatctggggccaagggacaatggtcaccgtctcttca

SEQ ID NO: 10 - DNA, 321 bp, Homo sapiens
gccatccggttgacccagtctccatcctccctgtctgcatctgtaggagacagagtcaccatctcttgccgggcaagtcagatcattagcaatcatttaaattggtatcagcagaaaccaggaaaagcccctaagctcctgatctatgctgcatccagattgcaaactggggtcccatcaaggttcagtggcagtggctctgagacagactacactctcaccatcagcagtctgcaacctgaagattttgcaacttactactgtcaacagagttacagtaacccccggacgttcggccacgggaccaaggtggaaatcaaa

SEQ ID NO: 11 - protein, 5 aa, Homo sapiens
SNYMS

SEQ ID NO: 12 - protein, 16 aa, Homo sapiens
VIYSGGSTYYADSVKG

SEQ ID NO: 13 - protein, 15 aa, Homo sapiens
GNPYYYGDLQVNFDY

SEQ ID NO: 14 - protein, 123 aa, Homo sapiens
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCARGNPYYYGDLQVNFDYWGQGTLVTVSS

SEQ ID NO: 15 - protein, 14 aa, Homo sapiens
GSSTGAVTSGNLPN

SEQ ID NO: 16 - protein, 7 aa, Homo sapiens
STSNKHS

SEQ ID NO: 17 - protein, 9 aa, Homo sapiens
LLYYGGAWV

SEQ ID NO: 18 - protein, 109 aa, Homo sapiens
QAVVTQEPSLTVSPGGTVTPTCGSSTGAVTSGNLPNWFQQKPGQAPRALIYSTSNKHSWTPARISGSLLGGKAALTLSGVQPEDEAEYYCLLYYGGAWVFGGGTKLTVL

SEQ ID NO: 19 - DNA, 369 bp, Homo sapiens
gaagtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagaggtaacccatattactacggtgacctccaggtgaactttgactactggggccagggaaccctggtcaccgtctcctca

SEQ ID NO: 20 - DNA, 327 bp, Homo sapiens
caggctgtggtgactcaggagccctcactgactgtgtccccaggagggacagtcactcccacctgtggttccagcactggagcagtcaccagtggtaaccttccaaactggttccagcagaaacctggacaagcacccagggcactgatttatagtacaagcaacaaacactcctggacccctgcccggatctcaggctccctccttgggggcaaagctgccctgacactgtcaggtgtgcagcctgaggacgaggctgaatattactgtctactctattatggtggtgcttgggtgttcggcggagggaccaagctgaccgtccta

SEQ ID NO: 21 - protein, 5 aa, Homo sapiens
SYAIS

SEQ ID NO: 22 - protein, 17 aa, Homo sapiens
GIIPIFGTANYAQKFQG

SEQ ID NO: 23 - protein, 13 aa, Homo sapiens
PRIQLWAAGGFDY

SEQ ID NO: 24 - protein, 121 aa, Homo sapiens
VQLVQSGAEVKKPGSSVKVSCKASGGTFSSYAISWVRQAPGQGLEWMGGIIPIFGTANYAQKFQGRVTITADESTSTAYMELSSLRSEDTAVYYCASPRIQLWAAGGFDYWGQGTLVTVSS

SEQ ID NO: 25 - protein, 14 aa, Homo sapiens
TGTSSDVGGYNLVS

SEQ ID NO: 26 - protein, 7 aa, Homo sapiens
DVSNRPS

SEQ ID NO: 27 - protein, 10 aa, Homo sapiens
SSFTTSTTLV

SEQ ID NO: 28 - protein, 110 aa, Homo sapiens
QSALTQPASVSGSPGQSITISCTGTSSDVGGYNLVSWYQQHPGKAPKLMIYDVSNRPSGVSNRFSGSKSGNTASLTISGLQAEDEADYYCSSFTTSTTLVFGGGTKLTVL

SEQ ID NO: 29 - DNA, 363 bp, Homo sapiens
gtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagccccaggatacagctatgggccgccgggggctttgactactggggccagggaaccctggtcactgtctcctca

SEQ ID NO: 30 - DNA, 330 bp, Homo sapiens
cagtctgccctgactcagcctgcctccgtgtctgggtctcctggacagtcgatcaccatctcctgcactggaaccagcagtgacgttggtggttataaccttgtctcctggtaccaacagcacccaggcaaagcccccaaactcatgatttatgatgtcagtaatcggccctcaggggtttctaatcgcttctctggctccaagtctggcaacacggcctccctgaccatctctgggctccaggctgaggacgaggctgattattattgcagctcatttacaactagcaccactctagtattcggcggagggaccaaactgaccgtccta

SEQ ID NO: 31 - protein, 5 aa, Homo sapiens
SYSMN

SEQ ID NO: 32 - protein, 17 aa, Homo sapiens
SISSSSSYIYYADSVKG

SEQ ID NO: 33 - protein, 17 aa, Homo sapiens
GQLLWFGESALIDAFDI

SEQ ID NO: 34 - protein, 126 aa, Homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGQLLWFGESALIDAFDIWGQGTMVTVSS

SEQ ID NO: 35 - protein, 11 aa, Homo sapiens
RASQSISSYLN

SEQ ID NO: 36 - protein, 7 aa, Homo sapiens
AASILQS

SEQ ID NO: 37 - protein, 9 aa, Homo sapiens
QQSYSTPRT

SEQ ID NO: 38 - protein, 107 aa, Homo sapiens
EIVLTQSPSSLSASVGDRVTITCRASQSISSYLNWYQLKPGKAPKLLIYAASILQSGVPSRFSGSGSGTDFTLTISSLQPEDFAAYYCQQSYSTPRTFGQGTKLEIK

SEQ ID NO: 39 - DNA, 378 bp, Homo sapiens
gaagtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaggtcaattactatggttcggggagtcagcactgattgatgcttttgatatctggggccaagggacaatggtcaccgtctcttca

SEQ ID NO: 40 - DNA, 321 bp, Homo sapiens
gaaattgtgttgacgcagtctccatcctccctgtctgcatctgtaggagacagagtcaccatcacttgccgggcaagtcagagcattagcagctatttaaattggtatcagctgaaaccagggaaagcccctaaactcctgatctatgctgcatccattctgcaaagtggggtcccatcaaggttcagtggcagtggatctgggacagatttcactctcaccatcagcagtctgcaacctgaagattttgcagcttactactgtcaacagagttacagtacccctcggacttttggccaggggaccaagctggagatcaaa

SEQ ID NO: 41 - protein, 5 aa, Homo sapiens
SNYMS

SEQ ID NO: 42 - protein, 16 aa, Homo sapiens
VIYSGGSTYYADSVKG

SEQ ID NO: 43 - protein, 15 aa, Homo sapiens
GNPYYYGDLQVNFDY

SEQ ID NO: 44 - protein, 123 aa, Homo sapiens; identical to SEQ ID NO: 14

SEQ ID NO: 45 - protein, 14 aa, Homo sapiens
ASSTGAVTSGYYPN

SEQ ID NO: 46 - protein, 7 aa, Homo sapiens
STSNKDS

SEQ ID NO: 47 - protein, 9 aa, Homo sapiens
LLYYGGAWV

SEQ ID NO: 48 - protein, 109 aa, Homo sapiens
QAVVTQEPSLTVSPGETVTLTCASSTGAVTSGYYPNWFQQRPGQAPRALIYSTSNKDSWTPARFSGSLLGGKAALTLSGVQPEDEAEYYCLLYYGGAWVFGGGTKLTVL

SEQ ID NO: 49 - DNA, 369 bp, Homo sapiens
gaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagaggtaacccatattactacggtgacctccaggtgaactttgactactggggccagggaaccctggtcaccgtctcctca

SEQ ID NO: 50 - DNA, 327 bp, Homo sapiens
caggctgtggtgactcaggagccctcactgactgtgtccccaggagagacggtcactctcacctgtgcttccagcactggagcagtcaccagtggttactatccaaactggttccagcagagacctggacaagcacccagggcactgatttatagtacaagcaacaaagactcctggacccctgcccggttctcaggctccctccttgggggcaaagctgccctgacactgtcaggtgtgcagcctgaggacgaggctgagtattactgcctgctctactatggtggtgcttgggtgttcggcggagggaccaagctgaccgtccta

SEQ ID NO: 51 - protein, 5 aa, Homo sapiens
SNYMS

SEQ ID NO: 52 - protein, 16 aa, Homo sapiens
VIYSGGSTYYADSVKG

SEQ ID NO: 53 - protein, 15 aa, Homo sapiens
GNPYYYGDLQVNFDY

SEQ ID NO: 54 - protein, 123 aa, Homo sapiens; identical to SEQ ID NO: 14

SEQ ID NO: 55 - protein, 14 aa, Homo sapiens
ASSTGAVTSGYYPN

SEQ ID NO: 56 - protein, 7 aa, Homo sapiens
STSNKHS

SEQ ID NO: 57 - protein, 9 aa, Homo sapiens
LLYYGGDWV

SEQ ID NO: 58 - protein, 109 aa, Homo sapiens
QAVVTQEPSLTVSPGGTVTLTCASSTGAVTSGYYPNWFQQKPGQAPRPLIYSTSNKHSWTPARFSGSLLGGKAALTLSGVQPDDEAEYYCLLYYGGDWVFGGGTKLTVL

SEQ ID NO: 59 - DNA, 369 bp, Homo sapiens; identical to SEQ ID NO: 49

SEQ ID NO: 60 - DNA, 327 bp, Homo sapiens
caggctgtggtgactcaggagccctcactgactgtgtccccaggagggacagtcactctcacctgtgcttccagcactggagcagtcaccagtggttactatccaaactggttccagcagaaacctggacaagcacccaggccactgatttatagtacaagcaacaaacactcctggacccctgcccggttctcaggctccctccttgggggcaaagctgccctgacactgtcaggtgtgcagcctgacgacgaggctgaatattactgcctgctctactatggtggtgactgggtgttcggcggagggaccaagctgaccgtccta

SEQ ID NO: 61 - protein, 5 aa, Homo sapiens
GYYMH

SEQ ID NO: 62 - protein, 17 aa, Homo sapiens
WINPNSGGTNYAQKFQG

SEQ ID NO: 63 - protein, 6 aa, Homo sapiens
GSNFDY

SEQ ID NO: 64 - protein, 115 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAAGSNFDYWGQGTLVTVSS

SEQ ID NO: 65 - protein, 14 aa, Homo sapiens
TGSSSDIGSFSYVS

SEQ ID NO: 66 - protein, 7 aa, Homo sapiens
GVNNRPL

SEQ ID NO: 67 - protein, 10 aa, Homo sapiens
SSYTRRSTVI

SEQ ID NO: 68 - protein, 110 aa, Homo sapiens
QSALTQPASVSGSPGQSITIACTGSSSDIGSFSYVSWYQQRPGKAPTLIIYGVNNRPLGVSRRFSGSKSGNTASLSISGLQAEDEADYYCSSYTRRSTVIFGGGTKLTVL

SEQ ID NO: 69 - DNA, 345 bp, Homo sapiens
caggttcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgctgcggggtctaactttgactactggggccagggaaccctggtcaccgtctcctca

SEQ ID NO: 70 - DNA, 330 bp, Homo sapiens
cagtctgccctgactcagcctgcctccgtgtctgggtctccgggacaatcgatcaccatcgcctgcactggaagcagtagtgacattggtagtttttcttatgtctcctggtaccaacagcgccccggcaaagcccccacactcatcatttatggggtcaataatcgacccttaggggtgtctcggcgcttctctggctccaagtctggcaacacggcctccctaagcatctctgggctccaggctgaggacgaggctgattattactgcagttcctatacacgcagaagcactgtgatcttcggcggcgggaccaagttgaccgtccta

SEQ ID NO: 71 - protein, 7 aa, Homo sapiens
SSGYYWG

SEQ ID NO: 72 - protein, 16 aa, Homo sapiens
SIYYSGSTYYNPSLKS

SEQ ID NO: 73 - protein, 13 aa, Homo sapiens
HRVRFGEFDAFDI

SEQ ID NO: 74 - protein, 123 aa, Homo sapiens
QLQLQESGPGLVKPSETLSLTCTVSGGSISSSGYYWGWIRQPPGKGLEWIGSIYYSGSTYYNPSLKSRVTISVDTSKNQFSLKLSSVTAADTAVYYCARHRVRFGEFDAFDIWGQGTMVTVSS

SEQ ID NO: 75 - protein, 14 aa, Homo sapiens
TGTSSNVGADFDVH

SEQ ID NO: 76 - protein, 7 aa, Homo sapiens
GSDNRPS

SEQ ID NO: 77 - protein, 11 aa, Homo sapiens
QAYDVRLSGWV

SEQ ID NO: 78 - protein, 111 aa, Homo sapiens
QAVLTQPPSVSGAPGERVTLSCTGTSSNVGADFDVHWYQQFPGTAPRLLIFGSDNRPSGVPDRFSGSKSGTSASLAITGLQVEDEADYYCQAYDVRLSGWVFGGGTKLTVL

SEQ ID NO: 79 - DNA, 369 bp, Homo sapiens
cagctgcagctgcaggagtcgggcccaggactggtgaagccttcggagaccctgtccctcacctgcaccgtctctggtggctccatcagcagtagtggttactactggggctggatccgccagcccccagggaaggggctggagtggattgggagtatctattatagtgggagcacctactacaacccgtccctcaagagtcgagtcaccatatccgtagacacgtctaagaaccagttctccctgaagctgagctctgtgaccgccgcagacacggctgtgtattactgtgcgagacatagggtacggttcggggagttcgatgcttttgatatctggggccaagggacaatggtcaccgtctcttca

SEQ ID NO: 80 - DNA, 333 bp, Homo sapiens
caggctgtgctgactcagccaccctcagtgtctggggcccccggagagagggtcaccctctcttgtacagggaccagctcgaacgtcggggcagattttgatgtacattggtaccagcagtttccaggaacagcccccagactcctcatctttggttccgacaatcggccctcaggagtccctgaccgattctctggctccaagtctggcacctcagcctccctggccatcactgggctccaggttgaggatgaggctgattattattgccaggcttatgacgtcaggctgagtggctgggtgttcggcggggggaccaagctgaccgtccta

SEQ ID NO: 81 - protein, 5 aa, Homo sapiens
SYAIS

SEQ ID NO: 82 - protein, 17 aa, Homo sapiens
GIIPIFGTANYAQKFQG

SEQ ID NO: 83 - protein, 11 aa, Homo sapiens
GRGFGELYFDY

SEQ ID NO: 84 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGRGFGELYFDYWGQGTLVTVSS

SEQ ID NO: 85 - protein, 11 aa, Homo sapiens
RASQGVRSNIA

SEQ ID NO: 86 - protein, 7 aa, Homo sapiens
DSSTRAT

SEQ ID NO: 87 - protein, 9 aa, Homo sapiens
QQYKNWPRT

SEQ ID NO: 88 - protein, 107 aa, Homo sapiens
ETTLTQSPATLSVSPGERATLSCRASQGVRSNIAWYQQKPGQAPRLLIYDSSTRATGIPARFSGSGSGTEFTLTISSLQSEDFAVYSCQQYKNWPRTFGQGTKVEIK

SEQ ID NO: 89 - DNA, 360 bp, Homo sapiens
caggttcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagggcgagggttcggggagttatactttgactactggggccagggaaccctggtcaccgtctcctca

SEQ ID NO: 90 - DNA, 321 bp, Homo sapiens
gaaacgacactcacgcagtctccagccaccctgtctgtgtctccaggggaaagagccaccctctcctgcagggccagtcagggtgttagaagcaatatagcctggtaccagcagaaacctggccaggctcccaggctcctcatctatgattcatccaccagggccactggtatcccagccaggttcagtggcagtgggtctgggacagagttcactctcaccatcagcagcctgcagtctgaagattttgcagtttattcctgtcagcagtataagaactggcctcggacgttcggccaagggaccaaggtggaaatcaaa

SEQ ID NO: 91 - protein, 5 aa, Homo sapiens
SYGIS

SEQ ID NO: 92 - protein, 17 aa, Homo sapiens
WISAYNGNTNYAQKLQG

SEQ ID NO: 93 - protein, 10 aa, Homo sapiens
VPAWSGQFDY

SEQ ID NO: 94 - protein, 119 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARVPAWSGQFDYWGQGTLVTVSS

SEQ ID NO: 95 - protein, 14 aa, Homo sapiens
TGSSSDVGGYNYVS

SEQ ID NO: 96 - protein, 7 aa, Homo sapiens
DVRSRPS

SEQ ID NO: 97 - protein, 11 aa, Homo sapiens
TSYTSSNTLVI

SEQ ID NO: 98 - protein, 111 aa, Homo sapiens
QSALTQPASVSGSPGQSITISCTGSSSDVGGYNYVSWYQQYPGKAPKLMIYDVRSRPSGVSNRFSGSKSGNTASLTISGLQAEDEADYYCTSYTSSNTLVIFGGGTKVTVL

SEQ ID NO: 99 - DNA, 357 bp, Homo sapiens
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcctacaatggtaacacaaactatgcacagaagctccagggcagagtcaccatgaccacagacacatccacgagcacagcctacatggagctgaggagcctgagatctgacgacacggccgtgtattactgtgcgagagtccctgcgtggagtggtcaatttgactactggggccagggaaccctggtcaccgtctcctca

SEQ ID NO: 100 - DNA, 333 bp, Homo sapiens
cagtctgccctgactcagcctgcctccgtgtctgggtctcctggacagtcgatcaccatctcctgcactggatccagcagtgacgttggtggttataactatgtctcctggtaccagcagtacccaggcaaagcccccaaactcatgatttatgatgtcagaagtcggccctcaggggtttctaatcgcttctctggctccaagtctggcaacacggcctccctgaccatctctggactccaggctgaggacgaggctgattattactgcacctcatatacaagcagcaacactcttgtgatattcggcggagggaccaaggtgaccgtccta

SEQ ID NO: 101 - protein, 5 aa, Homo sapiens
SYAMH

SEQ ID NO: 102 - protein, 17 aa, Homo sapiens
VISYDGSNKYYADSVKG

SEQ ID NO: 103 - protein, 11 aa, Homo sapiens
GGALNYYGMDV

SEQ ID NO: 104 - protein, 120 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAEGGALNYYGMDVWGQGTTVTVSS

SEQ ID NO: 105 - protein, 11 aa, Homo sapiens
QGDSLRHFYAT

SEQ ID NO: 106 - protein, 7 aa, Homo sapiens
GKNNRPS

SEQ ID NO: 107 - protein, 11 aa, Homo sapiens
QSRDPRNNHLI

SEQ ID NO: 108 - protein, 108 aa, Homo sapiens
SSELTQDPGVSVALGQTVRITCQGDSLRHFYATWYQQKPGQAPILVIYGKNNRPSGIPDRFSGSSSEDTASLTITGAQAEDEADYYCQSRDPRNNHLIFGGGTKLIVL

SEQ ID NO: 109 - DNA, 360 bp, Homo sapiens
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcggagggcggagcgttgaactactacggtatggacgtctggggccaagggaccacggtcaccgtctcctca

SEQ ID NO: 110 - DNA, 324 bp, Homo sapiens
tcttctgagctgactcaggaccctggtgtgtctgtggccttgggacagacagtcaggatcacatgccagggagacagcctcagacacttttatgcaacctggtaccagcagaagccaggacaggcccctatacttgtcatctatggtaaaaacaaccggccctcagggatcccagaccgattctctggctccagttctgaagacacagcctccttgaccatcactggggctcaggcggaggatgaggctgactactactgtcaatcccgggaccccaggaataaccatttgattttcggcggcgggaccaaactgatcgtcctc

SEQ ID NO: 111 - protein, 5 aa, Homo sapiens
SNYMS

SEQ ID NO: 112 - protein, 16 aa, Homo sapiens
VIYSGGSTYYADSVKG

SEQ ID NO: 113 - protein, 15 aa, Homo sapiens
GNPYYYGDLQVNFDY

SEQ ID NO: 114 - protein, 123 aa, Homo sapiens
EVQLLESGGGLIQPGGSLRLSCAVSGFTVSSNYMSWVRQAPGKGLEWVSVIYSGGSTYYADSVKGRFTISRDNSKNTLYLQMNSLRAEDTAVYYCARGNPYYYGDLQVNFDYWGQGTLVTVSS

SEQ ID NO: 115 - protein, 14 aa, Homo sapiens
ASSTGAVTSGYYPN

SEQ ID NO: 116 - protein, 7 aa, Homo sapiens
STSNKHS

SEQ ID NO: 117 - protein, 9 aa, Homo sapiens
LLYYGGAWV

SEQ ID NO: 118 - protein, 109 aa, Homo sapiens
QAVVTQEPSLTVSPGGTVTLTCASSTGAVTSGYYPNWFQQKPGQAPRALIYSTSNKHSWTPARFSGSLLGGKAALTLSGVQPEDEAEYYCLLYYGGAWVFGGGTKLTVL

SEQ ID NO: 119 - DNA, 369 bp, Homo sapiens
gaggtgcagctgttggagtctggaggaggcttgatccagcctggggggtccctgagactctcctgtgcagtctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagaggtaacccatattactacggtgacctccaggtgaactttgactactggggccagggaaccctggtcaccgtctcctca

SEQ ID NO: 120 - DNA, 327 bp, Homo sapiens
caggctgtggtgactcaggagccctcactgactgtgtccccaggagggacagtcactctcacctgtgcttccagcactggagcagtcaccagtggttactatccaaactggttccagcagaaacctggacaagcacccagggcactgatttatagtacaagcaacaaacactcctggacccctgcccggttctcaggctccctccttgggggcaaagctgccctgacactgtcaggtgtgcagcctgaggacgaggctgagtattactgcctgctctactatggtggtgcttgggtgttcggcggagggaccaagctgaccgtccta

SEQ ID NO: 121 - protein, 5 aa, Homo sapiens
SNYMS

SEQ ID NO: 122 - protein, 16 aa, Homo sapiens
VIYSGGSTYYADSVKG

SEQ ID NO: 123 - protein, 15 aa, Homo sapiens
GNPYYYGDLQVNFDY

SEQ ID NO: 124 - protein, 123 aa, Homo sapiens; identical to SEQ ID NO: 14

SEQ ID NO: 125 - protein, 14 aa, Homo sapiens
ASSTGAVTSAYYPN

SEQ ID NO: 126 - protein, 7 aa, Homo sapiens
STSNKHS

SEQ ID NO: 127 - protein, 9 aa, Homo sapiens
LLYYGGAWV

SEQ ID NO: 128 - protein, 109 aa, Homo sapiens
QAVVTQEPSLTVSPGGTVTLTCASSTGAVTSAYYPNWFQQKPGQAPRALIYSTSNKHSWTPARFSGSLLGGKAALTLSGVQPEDEADYYCLLYYGGAWVFGGGTKLTVL

SEQ ID NO: 129 - DNA, 369 bp, Homo sapiens; identical to SEQ ID NO: 19

SEQ ID NO: 130 - DNA, 327 bp, Homo sapiens
caggctgtggtgactcaggagccctcactgactgtgtccccaggagggacagtcactctcacctgtgcttccagcactggagcagtcaccagtgcttactatccaaactggttccagcagaaacctggacaagcacccagggcactgatttatagtacaagcaacaaacactcctggacccctgcccggttctcaggctccctccttgggggcaaagctgccctgacactgtcaggtgtgcagcctgaggacgaggctgactattactgcctgctctactatggtggtgcttgggtgttcggcggagggaccaagctgaccgtccta

SEQ ID NO: 131 - protein, 5 aa, Homo sapiens
DYYIH

SEQ ID NO: 132 - protein, 17 aa, Homo sapiens
WFNPSTGGANYAQKFQG

SEQ ID NO: 133 - protein, 8 aa, Homo sapiens
GNSPDLDY

SEQ ID NO: 134 - protein, 117 aa, Homo sapiens
QVQLVQSGAEVKKPGASVKVSCKASGYTFNDYYIHWVRQAPGQGLEWMGWFNPSTGGANYAQKFQGRVTMTRDTSISTAYLEVSSLRSDDTAVYYCARGNSPDLDYWGQGTLVTVSS

SEQ ID NO: 135 - protein, 17 aa, Homo sapiens
KSSQSLLHSSNNKNYLA

SEQ ID NO: 136 - protein, 7 aa, Homo sapiens
WASTRQS

SEQ ID NO: 137 - protein, 10 aa, Homo sapiens
QQYYTTTPNT

SEQ ID NO: 138 - protein, 114 aa, Homo sapiens
DIQMTQSPDSLTVSLGERATINCKSSQSLLHSSNNKNYLAWYQQRPGQPPKLLIYWASTRQSGVPDRFSGSGSGTDFTLTINSLQADDMAVYYCQQYYTTTPNTFGQGTKLEIK

SEQ ID NO: 139 - DNA, 351 bp, Homo sapiens
caagtccagctggtgcagtctggggctgaggtgaagaagcctggggcctcagtgaaggtctcctgcaaggcttctggatacaccttcaacgactactatatacactgggtgcggcaggcccctggacaagggcttgagtggatggggtggttcaaccctagcactggtggcgcaaattatgcacagaagtttcagggcagggtcaccatgaccagggacacgtccatcagcacagcctacttggaagtgagcagcctgagatctgacgacacggccgtgtattactgtgcgagaggtaatagcccggaccttgactactggggccagggaaccctggtcaccgtctcctca

SEQ ID NO: 140 - DNA, 342 bp, Homo sapiens
gacatccagatgacccagtctccagactccctgactgtgtctctgggcgagagggccaccatcaactgcaagtcgagccagagtcttttacacagctccaacaataagaattacttggcttggtaccagcagagaccaggacagcctcctaaactgctcatttactgggcatccacccggcaatccggggtcccggaccgcttcagtggcagcgggtctgggacagatttcactctcaccatcaacagcctgcaggctgacgacatggcagtttattactgccagcagtattatactactactccgaacacttttggccaggggaccaagctggagatcaaa

SEQ ID NO: 141 - protein, 5 aa, Homo sapiens
GYYMH

SEQ ID NO: 142 - protein, 17 aa, Homo sapiens
WINPNSGGTNYAQKFQG

SEQ ID NO: 143 - protein, 14 aa, Homo sapiens
DPDGSGGSSRWFDP

SEQ ID NO: 144 - protein, 123 aa, Homo sapiens
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDPDGSGGSSRWFDPWGQGTLVTVSS

SEQ ID NO: 145 - protein, 12 aa, Homo sapiens
TLSSGHTNYAIA

SEQ ID NO: 146 - protein, 11 aa, Homo sapiens
LNSDGSHTRGG

SEQ ID NO: 147 - protein, 9 aa, Homo sapiens
MIWHNNAWV

SEQ ID NO: 148 - protein, 111 aa, Homo sapiens
QPVLTQSPSASASLGASVKLTCTLSSGHTNYAIAWRQQQPGKAPRYLMLLNSDGSHTRGGGIPDRFSGSSSGAERYLTISSLQSEDEADYYCMIWHNNAWVFGGGTKLAVL

SEQ ID NO: 149 - DNA, 369 bp, Homo sapiens
caagtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagatcctgatgggagtggtggtagttcccggtggttcgacccctggggccagggaaccctggtcaccgtctcctca

SEQ ID NO: 150 - DNA, 333 bp, Homo sapiens
cagcctgtgctgactcaatcgccctctgcctctgcctccctgggagcctcggtcaagctcacctgcactctgagcagtgggcacaccaactacgccatcgcttggcgtcagcaacagcctgggaaggcccctcgatatttgatgctgcttaacagtgatggcagccacacgagggggggcgggatccctgatcgcttctcaggctccagttctggggctgagcgctacctcaccatctccagcctccagtctgaggatgaggctgactattactgtatgatttggcacaacaacgcttgggtgttcggcggagggaccaagctggccgtcctt

SEQ ID NO: 151 - protein, 5 aa, Homo sapiens
GYYMH

SEQ ID NO: 152 - protein, 17 aa, Homo sapiens
GIIPIFGTANYAQKFQG

SEQ ID NO: 153 - protein, 18 aa, Homo sapiens
AGMELTRSGAYYYYGMDV

SEQ ID NO: 154 - protein, 127 aa, Homo sapiens
QVQLQESGAEVKKPGASVKVSCKASGYTFTGYYMHWVRQAPGQGLEWMGGIIPIFGTANYAQKFQGRVTITADESTSTAYMELSSLRSEDTAVYYCAGAGMELTRSGAYYYYGMDVWGQGTTVTVSS

SEQ ID NO: 155 - protein, 14 aa, Homo sapiens
TGTSSDIGGYTFVS

SEQ ID NO: 156 - protein, 7 aa, Homo sapiens
DVNNRPS

SEQ ID NO: 157 - protein, 10 aa, Homo sapiens
SSVTSTNTYV

SEQ ID NO: 158 - protein, 110 aa, Homo sapiens
QSALTQPASVSGSPGQSITISCTGTSSDIGGYTFVSWYQQHPGKAPKVMIHDVNNRPSGISNRFSGSKSGNTASLTISGLQAEDEADYYCSSVTSTNTYVFGTGTKVTVL

SEQ ID NO: 159 - DNA, 381 bp, Homo sapiens
caggtgcagctgcaggagtcgggggctgaggtgaagaagcctggggcctcagtgaaggtctcctgcaaggcttctggatacaccttcaccggctactatatgcactgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgccggggcggggatggagcttactcgctcgggtgcttactactactacggtatggacgtctggggccaagggaccacggtcaccgtctcctca

SEQ ID NO: 160 - DNA, 330 bp, Homo sapiens
cagtctgccctgactcagcctgcctccgtgtctgggtctcctggacagtcgatcaccatctcctgcactggaaccagcagtgacattggtggttatacctttgtctcctggtaccaacaacacccaggcaaagcccccaaagtcatgattcatgatgtcaataatcggccctcagggatttctaatcgcttctctggctccaagtctggcaacacggcctccctgaccatctctgggctccaggctgaggacgaggctgattattactgcagctcagttacaagcaccaacacttatgtcttcggaactgggacaaaggtcaccgtccta

SEQ ID NO: 161 - protein, 5 aa, Homo sapiens
GYYMH

SEQ ID NO: 162 - protein, 17 aa, Homo sapiens
WINPNSGNTGYAQKFQG

SEQ ID NO: 163 - protein, 11 aa, Homo sapiens
GGQQQLVLDDY

SEQ ID NO: 164 - protein, 120 aa, Homo sapiens
QVQLVQSGAEVKKPGASVKVSCKASGYTFTGYYMHWVRQAPGQGLEWMGWINPNSGNTGYAQKFQGRVTMTRNTSISTAYMELSSLRSEDTAVYYCARGGQQQLVLDDYWGQGTLVTVSS

SEQ ID NO: 165 - protein, 11 aa, Homo sapiens
QASQDIRHHLN

SEQ ID NO: 166 - protein, 7 aa, Homo sapiens
DSSNLET

SEQ ID NO: 167 - protein, 9 aa, Homo sapiens
QQYDSLPRT

SEQ ID NO: 168 - protein, 107 aa, Homo sapiens
DIVLTQSPASLSASVGDRITITCQASQDIRHHLNWFQHKPGKAPKLLISDSSNLETGVPSRFSGSGSGTDFSFTISRLQPEDIATYYCQQYDSLPRTFGQGTKLEIK

SEQ ID NO: 169 - DNA, 360 bp, Homo sapiens
caggtgcagctggtgcaatctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtaacacaggctatgcacagaagttccagggcagagtcaccatgaccaggaacacctccataagcacagcctacatggagctgagcagcctgagatctgaggacacggccgtgtattactgtgcgagaggcggacagcagcagctggtactggacgactactggggccagggaaccctggtcaccgtctcctca

SEQ ID NO: 170 - DNA, 321 bp, Homo sapiens
gatattgtgctgacacagtctccagcctccctgtctgcatctgtcggcgacaggatcaccatcacctgccaggcgagtcaggacattaggcatcatttaaattggtttcagcacaaaccagggaaagcccccaagctcctgatctccgattcatccaacctggaaacaggagtcccgtcaaggttcagtggaagtgggtctgggacagatttttctttcaccatcagccgcctgcagcctgaagatattgcgacttattactgtcaacaatatgatagtctgcctcgaacctttggccaggggaccaaactggagatcaaa

SEQ ID NO: 171 - protein, 5 aa, Homo sapiens
SYAMH

SEQ ID NO: 172 - protein, 17 aa, Homo sapiens
VISYDGSNKYYADSVKG

SEQ ID NO: 173 - protein, 12 aa, Homo sapiens
ASPSQWLVLGHY

SEQ ID NO: 174 - protein, 121 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARASPSQWLVLGHYWGQGTLVTVSS

SEQ ID NO: 175 - protein, 12 aa, Homo sapiens
RASQSVSSSYLA

SEQ ID NO: 176 - protein, 7 aa, Homo sapiens
GASSRAT

SEQ ID NO: 177 - protein, 9 aa, Homo sapiens
QQYGSSPWT

SEQ ID NO: 178 - protein, 108 aa, Homo sapiens
ETTLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 179 - DNA, 363 bp, Homo sapiens
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagcctctccatcacagtggctggtactcgggcactactggggccagggaaccctggtcaccgtctcctca

SEQ ID NO: 180 - DNA, 324 bp, Homo sapiens
gaaacgacactcacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaa

SEQ ID NO: 181 - protein, 5 aa, Homo sapiens
GYYMH

SEQ ID NO: 182 - protein, 17 aa, Homo sapiens
WINPNSGNTGYAQKFQG

SEQ ID NO: 183 - protein, 11 aa, Homo sapiens
GGQQQLVLDDY

SEQ ID NO: 184 - protein, 120 aa, Homo sapiens; identical to SEQ ID NO: 164

SEQ ID NO: 185 - protein, 11 aa, Homo sapiens
RASQSVSSNLA

SEQ ID NO: 186 - protein, 7 aa, Homo sapiens
GASTRAT

SEQ ID NO: 187 - protein, 9 aa, Homo sapiens
QQYNNWPWT

SEQ ID NO: 188 - protein, 107 aa, Homo sapiens
ETTLTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVETK

SEQ ID NO: 189 - DNA, 360 bp, Homo sapiens
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtaacacaggctatgcacagaagttccagggcagagtcaccatgaccaggaacacctccataagcacagcctacatggagctgagcagcctgagatctgaggacacggccgtgtattactgtgcgagaggcggacagcagcagctggtactggacgactactggggccagggaaccctggtcaccgtctcctca

SEQ ID NO: 190 - DNA, 321 bp, Homo sapiens
gaaacgacactcacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccttggacgttcggccaagggaccaaggtggaaaccaaa

SEQ ID NO: 191 - protein, 7 aa, Homo sapiens
SGGYYWS

SEQ ID NO: 192 - protein, 16 aa, Homo sapiens
YIYYSGSTYYNPSLKS

SEQ ID NO: 193 - protein, 18 aa, Homo sapiens
GGISPSGSSIYYYYGMDV

SEQ ID NO: 194 - protein, 128 aa, Homo sapiens
QLQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARGGISPSGSSIYYYYGMDVWGQGTTVTVSS

SEQ ID NO: 195 - protein, 14 aa, Homo sapiens
TGTSNDVGGYNYVS

SEQ ID NO: 196 - protein, 7 aa, Homo sapiens
EVNKRPS

SEQ ID NO: 197 - protein, 9 aa, Homo sapiens
SSYAGTKEV

SEQ ID NO: 198 - protein, 109 aa, Homo sapiens
QPVLTQPPSASGSPGQSVTISCTGTSNDVGGYNYVSWYQQHPGKAPKLIIYEVNKRPSGVPDRFSGSKSGNTASLTVSGLQAVDESDYYCSSYAGTKEVFGGGTKLTVL

SEQ ID NO: 199 - DNA, 384 bp, Homo sapiens
cagctgcagctgcaggagtccggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagaggtgggatatcaccctccgggtcctcgatctactactactacggtatggacgtctggggccaagggaccacggtcaccgtctcctca

SEQ ID NO: 200 - DNA, 327 bp, Homo sapiens
cagcctgtgctgactcagccaccctccgcgtccgggtctcctggacagtcagtcaccatctcctgcactggaaccagcaatgacgttggtggttataactatgtctcctggtaccaacaacacccaggcaaagcccccaaactcataatttatgaggtcaataagcggccctcaggggtccctgatcgcttctctggctccaagtctggcaacacggcctccctgaccgtctctgggctccaggctgtggatgagtctgattattactgcagctcatatgcaggcaccaaggaggtcttcggcggagggaccaagctgaccgtccta

SEQ ID NO: 201 - protein, 5 aa, Homo sapiens
SYGIS

SEQ ID NO: 202 - protein, 17 aa, Homo sapiens
WISAYNGNTNYAQKLQG

SEQ ID NO: 203 - protein, 10 aa, Homo sapiens
VPAWSGQFDY

SEQ ID NO: 204 - protein, 119 aa, Homo sapiens; identical to SEQ ID NO: 94

SEQ ID NO: 205 - protein, 14 aa, Homo sapiens
TGTSSDVGGYNYVS

SEQ ID NO: 206 - protein, 7 aa, Homo sapiens
EVTNRPS

SEQ ID NO: 207 - protein, 11 aa, Homo sapiens
NSYTSGPTYVL

SEQ ID NO: 208 - protein, 111 aa, Homo sapiens
QSALTQPASVSGSPGQSITISCTGTSSDVGGYNYVSWYQQHPGKAPKLMIYEVTNRPSGVSDRFSGSKSANTASLTISELQAEDEAVYYCNSYTSGPTYVLFGGGTQLTVL

SEQ ID NO: 209 - DNA, 357 bp, Homo sapiens
caggtgcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagtccctgcgtggagtggtcaatttgactactggggccagggaaccctggtcaccgtctcctca

SEQ ID NO: 210 - DNA, 333 bp, Homo sapiens
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcactaatcggccctcaggggtttccgatcgcttctctggctccaagtctgccaacacggcctccctgaccatctctgagctccaggctgaagacgaggctgtttattactgcaactcatacacaagcggccccacttatgtgctgttcggcggagggacccagctgaccgtccta

SEQ ID NO: 211 - protein, 7 aa, Homo sapiens
SGGYYWS

SEQ ID NO: 212 - protein, 16 aa, Homo sapiens
YIYYSGSTYYNPSLKS

SEQ ID NO: 213 - protein, 11 aa, Homo sapiens
ASRSTDYYFDY

SEQ ID NO: 214 - protein, 121 aa, Homo sapiens
EVQLLESGPGLVKPSQTLSLTCTVSGGSISSGGYYWSWIRQHPGKGLEWIGYIYYSGSTYYNPSLKSRVTISVDTSKNQFSLKLSSVTAADTAVYYCARASRSTDYYFDYWGQGTLVTVSS

SEQ ID NO: 215 - protein, 13 aa, Homo sapiens
TGNRNNIGDQGAA

SEQ ID NO: 216 - protein, 7 aa, Homo sapiens
RNNNGPS

SEQ ID NO: 217 - protein, 11 aa, Homo sapiens
SAWDSSLRAWV

SEQ ID NO: 218 - protein, 110 aa, Homo sapiens
QPGLTQPPSMSYGLGQTATLTCTGNRNNIGDQGAAWLQQHQGHPPKLLSYRNNNGPSGISERLSASRSGNTASLTISGLQPEDEADYYCSAWDSSLRAWVFGGGTKLTVL

SEQ ID NO: 219 - DNA, 363 bp, Homo sapiens
gaagtgcagctgttggagtctggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagggcttcacgatcgaccgattactactttgactactggggccagggaaccctggtcaccgtctcctca

SEQ ID NO: 220 - DNA, 330 bp, Homo sapiens
cagccagggctgactcagccaccctcgatgtcctacggcttgggacagaccgccacactcacctgcactgggaacagaaacaatattggcgaccaaggagcagcttggctgcagcagcaccagggccaccctcccaaactcctatcctacaggaataacaacgggccctcagggatctcagagagattatctgcatccaggtcaggaaacacagcctccctgaccattagtggactccagcctgaggacgaggctgactattactgctcagcatgggacagcagcctcagggcttgggtgttcggcggagggaccaagctgaccgtcctc

SEQ ID NO: 221 - protein, 5 aa, Homo sapiens
SYAIS

SEQ ID NO: 222 - protein, 17 aa, Homo sapiens
GIIPIFGTANYAQKFQG

SEQ ID NO: 223 - protein, 15 aa, Homo sapiens
PKYSSGWFYYYGMDV

SEQ ID NO: 224 - protein, 124 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARPKYSSGWFYYYGMDVWGQGTTVTVSS

SEQ ID NO: 225 - protein, 13 aa, Homo sapiens
SGSSSNIGNNYVS

SEQ ID NO: 226 - protein, 7 aa, Homo sapiens
DNNKRPS

SEQ ID NO: 227 - protein, 10 aa, Homo sapiens
GTWDSSLSVV

SEQ ID NO: 228 - protein, 109 aa, Homo sapiens
QSVLTQPPSVSAAPGQKVTISCSGSSSNIGNNYVSWYQQLPGTAPKLLIYDNNKRPSGIPDRFSGSKSGTSATLGITGLQTGDEADYYCGTWDSSLSVVFGGGTKLTVL

SEQ ID NO: 229 - DNA, 372 bp, Homo sapiens
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaccaaagtatagcagtggctggttctactactacggtatggacgtctggggccaagggaccacggtcaccgtctcctca

SEQ ID NO: 230 - DNA, 327 bp, Homo sapiens
cagtctgtgctgactcagccaccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcaccggactccagactggggacgaggccgattattactgcggaacatgggatagcagcctgagtgtggtattcggcggagggaccaagctgaccgtccta

SEQ ID NO: 231 - protein, 5 aa, Homo sapiens
SYAIS

SEQ ID NO: 232 - protein, 17 aa, Homo sapiens
WISAYNGNTNYAQKLQG

SEQ ID NO: 233 - protein, 12 aa, Homo sapiens
LGSYGYTGAFDI

SEQ ID NO: 234 - protein, 121 aa, Homo sapiens
QMQLVQSGAEVKKPGSSVKVSCKASGGTFSSYAISWVRQAPGQGLEWMGWISAYNGNTNYAQKLQGRVTMTTDTSTSTAYMELRSLRSDDTAVYYCASLGSYGYTGAFDIWGQGTMATVSS

SEQ ID NO: 235 - protein, 14 aa, Homo sapiens
TGTSSDVGGYNYVS

SEQ ID NO: 236 - protein, 7 aa, Homo sapiens
EVSNRPS

SEQ ID NO: 237 - protein, 11 aa, Homo sapiens
SSYTSSSTLVV

SEQ ID NO: 238 - protein, 111 aa, Homo sapiens
QSALTQPASVSGSPGQSITISCTGTSSDVGGYNYVSWYQQHPGKAPKLMIYEVSNRPSGVSNRFSGSKSGNTASLTISGLQAEDEADYYCSSYTSSSTLVVFGGGTKLTVL

SEQ ID NO: 239 - DNA, 363 bp, Homo sapiens
caaatgcagctggtacaatctggggctgaggtgaagaagcctgggtcctcggtgaaggtctcctgcaaggcttctggaggcaccttcagcagctatgctatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagcctggggagctatggttatacaggggcttttgatatctggggccaagggacaatggccaccgtctcttca

SEQ ID NO: 240 - DNA, 333 bp, Homo sapiens
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcagcagcactctcgtggtattcggcggagggaccaagctgaccgtccta

SEQ ID NO: 241 - protein, 5 aa, Homo sapiens
SYAIS

SEQ ID NO: 242 - protein, 17 aa, Homo sapiens
GIIPIFGTANYAQKFQG

SEQ ID NO: 243 - protein, 11 aa, Homo sapiens
GGWLRQNWFDP

SEQ ID NO: 244 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELTSLRSEDTAVYYCARGGWLRQNWFDPWGQGTLVTVSS

SEQ ID NO: 245 - protein, 11 aa, Homo sapiens
QGDSLRSYYAS

SEQ ID NO: 246 - protein, 7 aa, Homo sapiens
GKNNRPS

SEQ ID NO: 247 - protein, 12 aa, Homo sapiens
NSRDSSGNHPRV

SEQ ID NO: 248 - protein, 109 aa, Homo sapiens
SSELTQDPAVSVALGQTVRITCQGDSLRSYYASWYQQKPGQAPVLVIYGKNNRPSGIPDRFSGSSSGNTASLTITGAQAEDEADYYCNSRDSSGNHPRVFGGGTKLTVL

SEQ ID NO: 249 - DNA, 360 bp, Homo sapiens
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgataaccgcggacgaatccacgagcacagcctacatggagctgaccagcctgagatctgaggacacggccgtgtattactgtgcgagaggggggtggctacgacagaactggttcgacccctggggccagggaaccctggtcaccgtctcctca

SEQ ID NO: 250 - DNA, 327 bp, Homo sapiens
tcttctgagctgactcaggaccctgctgtgtctgtggccttgggacaaacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccatccaagggtattcggcggagggaccaagctgaccgtccta

SEQ ID NO: 251 - protein, 7 aa, Homo sapiens
SGGYYWS

SEQ ID NO: 252 - protein, 16 aa, Homo sapiens
YIYYSGSTYYNPSLKS

SEQ ID NO: 253 - protein, 13 aa, Homo sapiens
WSLGTSNHGWFDP

SEQ ID NO: 254 - protein, 123 aa, Homo sapiens
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCASWSLGTSNHGWFDPWGQGTLVTVSS

SEQ ID NO: 255 - protein, 13 aa, Homo sapiens
SGSSSNIGSNTVN

SEQ ID NO: 256 - protein, 7 aa, Homo sapiens
DNNNRPS

SEQ ID NO: 257 - protein, 11 aa, Homo sapiens
QSYDSNLSGWV

SEQ ID NO: 258 - protein, 110 aa, Homo sapiens
QSVLTQPPSASGTPGQRVTISCSGSSSNIGSNTVNWYQQLPGTAPKLLISDNNNRPSGVPDRFSGSKSGTSASLAITGLQAEDEADYYCQSYDSNLSGWVFGGGTKLTVL

SEQ ID NO: 259 - DNA, 369 bp, Homo sapiens
caggtgcaactgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgaccgccgcagacacggctgtgtattactgtgcgagctggtccctaggtaccagcaaccatggttggttcgacccctggggccagggaaccctggtcaccgtctcctca

SEQ ID NO: 260 - DNA, 330 bp, Homo sapiens
cagtctgtgctgactcagccaccctcagcgtctgggacccccgggcagagggtcaccatctcttgttctggaagcagctccaacatcggaagtaatactgtaaactggtaccagcagcttccaggaacagcccccaaactcctcatctctgataataacaatcggccctcaggggtccctgaccgattctctggctccaagtctggcacctcagcctccctggccatcactgggctccaggctgaggatgaggctgattattactgccagtcctatgacagtaacctgagtggttgggtgttcggcggagggaccaagctgaccgtccta

SEQ ID NO: 261 - protein, 5 aa, Homo sapiens
SYAIS

SEQ ID NO: 262 - protein, 17 aa, Homo sapiens
GIIPIFGTANYAQKFQG

SEQ ID NO: 263 - protein, 11 aa, Homo sapiens
ARGSTWGYFDY

SEQ ID NO: 264 - protein, 120 aa, Homo sapiens
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCARARGSTWGYFDYWGQGTLVTVSS

SEQ ID NO: 265 - protein, 11 aa, Homo sapiens
RASQSVSNYLA

SEQ ID NO: 266 - protein, 7 aa, Homo sapiens
DASNRAT

SEQ ID NO: 267 - protein, 9 aa, Homo sapiens
QQRDNWPLT

SEQ ID NO: 268 - protein, 107 aa, Homo sapiens
EIVLTQSPASLSLSPGERVTLSCRASQSVSNYLAWYQQKPGQAPRLLIYDASNRATGIPARFSGSGSGTDFTLTISSLEPEDFAVYYCQQRDNWPLTFGGGTKVEIK

SEQ ID NO: 269 - DNA, 360 bp, Homo sapiens
caggtgcagctggtacaatctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtgtattactgtgcgagagcgaggggcagcacctggggctactttgactactggggccagggaaccctggtcaccgtctcctca

SEQ ID NO: 270 - DNA, 321 bp, Homo sapiens
gaaatagtgttgacgcagtctccagcctccctgtctttgtctccaggggaaagagtcaccctctcctgcagggccagtcagagtgttagcaattacttagcctggtatcaacagaaacctggccaggctcccaggctcctcatctatgatgcatccaacagggccactggcatcccagccaggttcagtggcagtgggtctgggacagacttcactctcaccatcagcagcctagagcccgaagattttgcagtttattactgtcagcagcgtgacaactggcccctcactttcggcggagggaccaaggtggagatcaaa

SEQ ID NO: 271 - protein, 5 aa, Homo sapiens
SYAIS

SEQ ID NO: 272 - protein, 17 aa, Homo sapiens
GIIPIFGTANYAQKFQG

SEQ ID NO: 273 - protein, 13 aa, Homo sapiens
VGVEYQLLWYFDY

SEQ ID NO: 274 - protein, 122 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARVGVEYQLLWYFDYWGQGTLVTVSS

SEQ ID NO: 275 - protein, 11 aa, Homo sapiens
RTSQTISNYLN

SEQ ID NO: 276 - protein, 7 aa, Homo sapiens
AASNLQS

SEQ ID NO: 277 - protein, 7 aa, Homo sapiens
QQSYNAS

SEQ ID NO: 278 - protein, 105 aa, Homo sapiens
DIQMTQSPSSLSASVGDRVTLTCRTSQTISNYLNWYQQKPGKAPKLLVYAASNLQSWVPSRFSGSGSGTDFTLTISSLQPEDFATYYCQQSYNASFGGGTKVEFK

SEQ ID NO: 279 - DNA, 366 bp, Homo sapiens
caagtgcagctggtacagtctggggctgaggtgaagaagcctgggtcctcggtgaaggtctcctgcaaggcttctggaggcaccttcagcagctatgcaatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagagtaggggttgagtaccagctgctatggtactttgactactggggccagggaaccctagtcaccgtctcctca

SEQ ID NO: 280 - DNA, 315 bp, Homo sapiens
gacatccagatgacccagtctccatcctccctgtctgcatctgtaggagacagagtcaccctcacttgccggacaagtcagaccattagcaactatttaaattggtatcagcagaaaccagggaaagcccctaagctcctggtctatgctgcatccaatttgcaaagttgggtcccatcaaggttcagtggcagtgggtctgggacagatttcactctcaccatcagcagtctgcagcctgaagattttgcaacttactactgtcaacagagttacaatgcctctttcggcggagggaccaaggtggagttcaaa

SEQ ID NO: 281 - protein, 5 aa, Homo sapiens
SYAIS

SEQ ID NO: 282 - protein, 17 aa, Homo sapiens
GIIPIFGTANYAQKFQG

SEQ ID NO: 283 - protein, 10 aa, Homo sapiens
SYYYYYGMDV

SEQ ID NO: 284 - protein, 119 aa, Homo sapiens
QVQLVQSGAEVKKPGSSVKVSCKASGGTFSSYAISWVRQAPGQGLEWMGGIIPIFGTANYAQKFQGRVTITADESTGTAYMELSSLRSEDTAVYYCARSYYYYYGMDVWGQGTTVTVSS

SEQ ID NO: 285 - protein, 16 aa, Homo sapiens
RSSQSLLHSNGYNYLD

SEQ ID NO: 286 - protein, 7 aa, Homo sapiens
LGSNRAS

SEQ ID NO: 287 - protein, 9 aa, Homo sapiens
MQAVDTPRT

SEQ ID NO: 288 - protein, 112 aa, Homo sapiens
DIQMTQSPLSLPVTPGEPASISCRSSQSLLHSNGYNYLDWYLQKPGQSPQLLIHLGSNRASGVPDRFSGSGSGTDFTLKISRVEAEDVGVYYCMQAVDTPRTFGQGTKVDIK

SEQ ID NO: 289 - DNA, 357 bp, Homo sapiens
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgggcacagcctacatggagctgagcagcctgagatctgaggacacggccgtgtattactgtgcgagaagttactactactactacggtatggacgtctggggccaagggaccacggtcaccgtctcctca

SEQ ID NO: 290 - DNA, 336 bp, Homo sapiens
gacatccagatgacccagtctccactctccctgcccgtcacccctggagagccggcctccatctcctgcaggtctagtcagagcctcctgcatagtaatggatacaactatttggattggtacctgcagaagccagggcagtcaccacagctcctgatccatttgggttctaatcgggcctccggggtccctgacaggttcagtggcagtggatcaggcacagattttacactgaaaattagcagagtggaggctgaggatgttggggtttattactgcatgcaagctgtagatactcctcggacgttcggccaagggaccaaggtggacatcaaa

SEQ ID NO: 291 - protein, 5 aa, Homo sapiens
GYYMH

SEQ ID NO: 292 - protein, 17 aa, Homo sapiens
WINPNSGNTGYAQKFQG

SEQ ID NO: 293 - protein, 11 aa, Homo sapiens
GGQQQLVLDDY

SEQ ID NO: 294 - protein, 120 aa, Homo sapiens; identical to SEQ ID NO: 164

SEQ ID NO: 295 - protein, 11 aa, Homo sapiens
RASQSIGGWLA

SEQ ID NO: 296 - protein, 7 aa, Homo sapiens
AASSLQS

SEQ ID NO: 297 - protein, 9 aa, Homo sapiens
RQSYSTPPT

SEQ ID NO: 298 - protein, 107 aa, Homo sapiens
DIQLTQSPSTLSASVGDRVTITCRASQSIGGWLAWYQHKPGKAPKLLIYAASSLQSGVPSRFSGSGSGTDFTLTISSLQPEDFATYYCRQSYSTPPTFGQGTKVEIK

SEQ ID NO: 299 - DNA, 360 bp, Homo sapiens; identical to SEQ ID NO: 189

SEQ ID NO: 300 - DNA, 321 bp, Homo sapiens
gacatccagttgacccagtctccttccaccctgtcagcatctgtaggcgacagagtcaccatcacttgccgggccagtcagtctattggtggttggttggcctggtatcagcacaaaccagggaaagcccccaagctcctgatctatgctgcatccagtttgcaaagtggggtcccatcaaggttcagtggcagtggatctgggacagatttcactctcaccatcagcagtctgcaacctgaagattttgcaacttactactgtcgacagagttacagtacccctccgacgttcggccaagggaccaaggtggaaatcaaa

SEQ ID NO: 301 - protein, 5 aa, Homo sapiens
SYAMH

SEQ ID NO: 302 - protein, 17 aa, Homo sapiens
VISYDGSNKYYADSVKG

SEQ ID NO: 303 - protein, 13 aa, Homo sapiens
VVAAADLTRYFDY

SEQ ID NO: 304 - protein, 122 aa, Homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARVVAAADLTRYFDYWGQGTLVTVSS

SEQ ID NO: 305 - protein, 12 aa, Homo sapiens
RASQSVPKNYLA

SEQ ID NO: 306 - protein, 7 aa, Homo sapiens
TASSRAP

SEQ ID NO: 307 - protein, 9 aa, Homo sapiens
QQYGTSPNT

SEQ ID NO: 308 - protein, 108 aa, Homo sapiens
EIVLTQSPGTVSLSPGERATLSCRASQSVPKNYLAWFQQKPGQAPRLVIHTASSRAPGIPDRFTGSGSGTDFTLTISRLEPEDFAVYYCQQYGTSPNTFGQGTKLDIK

SEQ ID NO: 309 - DNA, 366 bp, Homo sapiens
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagtcgtagcagcagctgacctcactcgctactttgactactggggccagggaaccctggtcaccgtctcctca

SEQ ID NO: 310 - DNA, 324 bp, Homo sapiens
gaaattgtgttgacgcagtctccaggcaccgtgtctttgtctccaggggaaagagccaccctctcctgcagggccagtcagagtgttcctaagaactacttagcctggttccagcagaaacctggccaggctcccaggctcgtcatccatactgcatccagcagggcccctggcatcccagacaggttcactggcagcgggtctgggacagacttcactcttaccatcagcagactggagcctgaagattttgcagtatattactgtcagcagtatggcacctcaccaaacacttttggccaggggaccaagctggacatcaaa

SEQ ID NO: 311 - protein, 5 aa, Homo sapiens
GYYMH

SEQ ID NO: 312 - protein, 17 aa, Homo sapiens
WMNPNSGNTGYAQKFQG

SEQ ID NO: 313 - protein, 11 aa, Homo sapiens
GGQQQLVLDDY

SEQ ID NO: 314 - protein, 120 aa, Homo sapiens
QVQLVQSGAEVKKPGASVKVSCKASGYTFTGYYMHWVRQAPGQGLEWMGWMNPNSGNTGYAQKFQGRVTMTRNTSISTAYMELSSLRSEDTAVYYCARGGQQQLVLDDYWGQGTLVTVSS

SEQ ID NO: 315 - protein, 16 aa, Homo sapiens
RSSQSLLHSNGYNYLD

SEQ ID NO: 316 - protein, 7 aa, Homo sapiens
LGSNRAS

SEQ ID NO: 317 - protein, 9 aa, Homo sapiens
MQALQTPRT

SEQ ID NO: 318 - protein, 112 aa, Homo sapiens
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPRTFGPGTKVDIK

SEQ ID NO: 319 - DNA, 360 bp, Homo sapiens
caagtgcagctggtacagtctggggctgaggtgaagaagcctggggcctcagtgaaggtctcctgcaaggcttctggatacaccttcaccggctactatatgcactgggtgcgacaggcccctggacaagggcttgagtggatggggtggatgaatcctaacagtggcaacacaggctatgcacagaagttccagggcagagtcaccatgaccaggaacacctccataagcacagcctacatggagctgagcagcctgagatctgaggacacggccgtgtattactgtgcgagaggcggacagcagcagctggtactggacgactactggggccagggaaccctggtcaccgtctcctca

SEQ ID NO: 320 - DNA, 336 bp, Homo sapiens
gacatcgtgatgacccagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaacccctcggactttcggccctgggaccaaagtggatatcaaa

SEQ ID NO: 321 - protein, 5 aa, Homo sapiens
SYGIS

SEQ ID NO: 322 - protein, 17 aa, Homo sapiens
WISAYNGNTNYAQKLQG

SEQ ID NO: 323 - protein, 12 aa, Homo sapiens
DHSIVGATTFDY

SEQ ID NO: 324 - protein, 121 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDHSIVGATTFDYWGQGTLVTVSS

SEQ ID NO: 325 - protein, 11 aa, Homo sapiens
RASQSISSWLA

SEQ ID NO: 326 - protein, 7 aa, Homo sapiens
DASSLES

SEQ ID NO: 327 - protein, 9 aa, Homo sapiens
QQYNSYPWT

SEQ ID NO: 328 - protein, 107 aa, Homo sapiens
DIVMTQSPSTLSASVGDRVTITCRASQSISSWLAWYQQKPGKAPKLLIYDASSLESGVPSRFSGSGSGTEFTLTISSLQPDDFATYYCQQYNSYPWTFGQGTKVEIK

SEQ ID NO: 329 - DNA, 363 bp, Homo sapiens
caggtccagctggtgcagtctggagctgaggtgaagaagcctggggcctcagtgaaggtctcctgcaaggcttctggttacacctttaccagctacggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatcacagtatagtgggagctactacgtttgactactggggccagggaaccctggtcaccgtctcctca

SEQ ID NO: 330 - DNA, 321 bp, Homo sapiens
gacatcgtgatgacccagtctccttccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgccaacagtacaatagttatccgtggacgttcggccaagggaccaaggtggaaatcaaa

SEQ ID NO: 331 - protein, 5 aa, Homo sapiens
SYTMN

SEQ ID NO: 332 - protein, 17 aa, Homo sapiens
SISSIGTYIYYADSVKG

SEQ ID NO: 333 - protein, 13 aa, Homo sapiens
VLLSGSYYGYFDS

SEQ ID NO: 334 - protein, 122 aa, Homo sapiens
QMQLVQSGGGLVKPGGSLRLSCAASGFTFSSYTMNWVRQAPGKGLEWVSSISSIGTYIYYADSVKGRFTISRDNAKNSLYLQMNSLRAEDTAVYYCARVLLSGSYYGYFDSWGQGTLVTVSS

SEQ ID NO: 335 - protein, 14 aa, Homo sapiens
TGSSSNIGAGYDVH

SEQ ID NO: 336 - protein, 7 aa, Homo sapiens
GNSNRPS

SEQ ID NO: 337 - protein, 11 aa, Homo sapiens
QSYDSSLSGYV

SEQ ID NO: 338 - protein, 111 aa, Homo sapiens
QSVLTQPPSVSGAPGQRVTISCTGSSSNIGAGYDVHWYQQLPGTAPKLFIYGNSNRPSGVPDRFSGSKSGTSASLAITGLQAEDEADYYCQSYDSSLSGYVFGTGTKVTVL

SEQ ID NO: 339 - DNA, 366 bp, Homo sapiens
cagatgcagctggtacagtctgggggaggcctggtcaagcctggggggtccctgagactctcctgtgcagcctctggattcaccttcagtagctataccatgaactgggtccgccaggctccagggaaggggctggagtgggtctcatccattagtagtattggtacttacatatactacgcagactcagtgaagggccgattcaccatctccagagacaacgccaagaactcactgtatctgcaaatgaacagcctgagagccgaggacacggctgtgtattactgtgcgagagtcctgttaagtgggagctactacggctactttgactcctggggccagggaaccctggtcaccgtctcctca

SEQ ID NO: 340 - DNA, 333 bp, Homo sapiens
cagtctgtgctgactcagccaccctcagtgtctggggccccagggcagagggtcaccatctcctgcactgggagcagctccaacatcggggcaggttatgatgtgcactggtaccagcagcttccaggaacagcccccaaactcttcatctatggtaacagcaatcggccctcaggggtccctgaccgattctctggctccaagtctggcacctcagcctccctggccatcactgggctccaggctgaggatgaggctgattattactgccagtcctatgacagcagcctgagtggttatgtcttcggaactgggaccaaggtcaccgtccta

SEQ ID NO: 341 - protein, 5 aa, Homo sapiens
GYYMH

SEQ ID NO: 342 - protein, 17 aa, Homo sapiens
WINPNSGNTGYAQKFQG

SEQ ID NO: 343 - protein, 12 aa, Homo sapiens
GRLERGYWYFDL

SEQ ID NO: 344 - protein, 121 aa, Homo sapiens
QVQLVQSGAEVKKPGASVKVSCKASGYTFTGYYMHWVRQAPGQGLEWMGWINPNSGNTGYAQKFQGRVTMTRNTSISTAYMELSSLRSEDTAVYYCARGRLERGYWYFDLWGRGTLVTVSS

SEQ ID NO: 345 - protein, 13 aa, Homo sapiens
TRSSGSITSNYVQ

SEQ ID NO: 346 - protein, 7 aa, Homo sapiens
EDKERPS

SEQ ID NO: 347 - protein, 11 aa, Homo sapiens
QSYGGTSQGVL

SEQ ID NO: 348 - protein, 112 aa, Homo sapiens
NFMLTQPHSVSESPGRTVTISCTRSSGSITSNYVQWYQQRPGSAPTILIYEDKERPSEVPDRFSGSIDISSNSASLTISGLKTEDEADYYCQSYGGTSQGVLFGGGTKVTVL

SEQ ID NO: 349 - DNA, 363 bp, Homo sapiens
caggtccagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtaacacaggctatgcacagaagttccagggcagagtcaccatgaccaggaacacctccataagcacagcctacatggagctgagcagcctgagatctgaggacacggccgtgtattactgtgcgagaggccgtctggaacgcggatactggtacttcgatctctggggccgtggcaccctggtcaccgtctcctca

SEQ ID NO: 350 - DNA, 336 bp, Homo sapiens
aattttatgctgactcagccccactctgtgtcggagtctccggggaggacggtaaccatctcctgcacccgcagcagtggcagcattaccagcaactatgtccagtggtaccagcagcgcccgggcagtgcccctaccatcctaatctatgaggataaggaaagaccctctgaggtccctgatcgcttctctggctccatcgacatttcctccaactctgcctccctcaccatctctggcctgaagacggaggacgaggctgactactactgtcagtcttatggtggcaccagtcaaggggtgttattcggcggagggaccaaggtgaccgtccta

SEQ ID NO: 351 - protein, 5 aa, Homo sapiens
GYYMH

SEQ ID NO: 352 - protein, 17 aa, Homo sapiens
WINPNSGGTNYAQKFQG

SEQ ID NO: 353 - protein, 11 aa, Homo sapiens
GGQQQLVLDDY

SEQ ID NO: 354 - protein, 120 aa, Homo sapiens
QVQLVQSGAEVKKPGASVKVSCKASGYTFTGYYMHWVRQAPGQGLEWMGWINPNSGGTNYAQKFQGRVTMTRNTSISTAYMELSSLRSEDTAVYYCARGGQQQLVLDDYWGQGTLVTVSS

SEQ ID NO: 355 - protein, 11 aa, Homo sapiens
RASQSISSYLN

SEQ ID NO: 356 - protein, 7 aa, Homo sapiens
AASSLQS

SEQ ID NO: 357 - protein, 9 aa, Homo sapiens
QQSYSTPRT

SEQ ID NO: 358 - protein, 107 aa, Homo sapiens
DIVMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 359 - DNA, 360 bp, Homo sapiens
caggtgcagctggtgcaatctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagttccagggcagagtcaccatgaccaggaacacctccataagcacagcctacatggagctgagcagcctgagatctgaggacacggccgtgtattactgtgcgagaggcggacagcagcagctggtactggacgactactggggccagggaaccctggtcaccgtctcctca

SEQ ID NO: 360 - DNA, 321 bp, Homo sapiens
gacatcgtgatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctcgaacgttcggccaagggaccaaggtggaaatcaaa

SEQ ID NO: 361 - protein, 5 aa, Homo sapiens
GYYMH

SEQ ID NO: 362 - protein, 17 aa, Homo sapiens
WINPNSGNTGYAQKFQG

SEQ ID NO: 363 - protein, 11 aa, Homo sapiens
GGQQQLVLDDY

SEQ ID NO: 364 - protein, 120 aa, Homo sapiens; identical to SEQ ID NO: 164

SEQ ID NO: 365 - protein, 13 aa, Homo sapiens
TGSSGSIASNYVQ

SEQ ID NO: 366 - protein, 7 aa, Homo sapiens
EDNQRPS

SEQ ID NO: 367 - protein, 10 aa, Homo sapiens
QSYDSSNQRV

SEQ ID NO: 368 - protein, 111 aa, Homo sapiens
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDSSNQRVFGGGTKLTVL

SEQ ID NO: 369 - DNA, 360 bp, Homo sapiens
caggttcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtaacacaggctatgcacagaaattccagggcagagtcaccatgaccaggaacacctccataagcacagcctacatggagctgagcagcctgagatctgaggacacggccgtgtattactgtgcgagaggcggacagcagcagctggtactggacgactactggggccagggaaccctggtcaccgtctcctca

SEQ ID NO: 370 - DNA, 333 bp, Homo sapiens
aattttatgctgactcagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaatcagagggtgttcggcggagggaccaagctgaccgtccta

SEQ ID NO: 371 - protein, 431 aa, Homo sapiens
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 372 - protein, 430 aa, Homo sapiens
MSLVELLLWWNCFSRTGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 373 - protein, 406 aa, Homo sapiens
MSLVELLLWWNCFSRTGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSAQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 374 - protein, 219 aa, Homo sapiens
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIG

SEQ ID NO: 375 - protein, 114 aa, Homo sapiens
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVT

SEQ ID NO: 376 - protein, 91 aa, Homo sapiens
PSAPHCQIQGSQDIGSDVILLCSSEEGIPRPTYLWEKLDNTLKLPPTATQDQVQGTVTIRNISALSSGLYQCVASNAIGTSTCLLDLQVIS

SEQ ID NO: 377 - protein, 431 aa, Homo sapiens
MTSRRSPLAPLLLLSLHGVAASLEVSESPGSIQVARGQTAVLPCTFTTSAALINLNVIWMVTPLSNANQPEQVILYQGGQMFDGAPRFHGRVGFTGTMPATNVSVFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISTLTSGLYQCVASNAIGTSTCLLDLQVISPQPRNIGLIAGAVGTGAVIIIFCIALILGAFFYWRSKNKEEEEEEIPNEIREDDLPPKCSSAKAFHTEISSSDNNTLTSSNTYNSRYWSNNPKVHRNTESVNHFSDLGQSFSLRSGNASIPSIYANGSHLLPGQHKTLVVTANRGSSPQVMSRSNGSVSRKPRPPHSHSYTISHATLERIGAVPVMVPAQSRAGSLV

SEQ ID NO: 378 - protein, 428 aa, Homo sapiens
MTRRRSAPASWLLVSLLGVATSLEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPQPRSVGVIAGAVGTGAVLIVICLALISGAFFYWRSKNKEEEEEEIPNEIREDDLPPKCSSAKAFHTEISSSENNTLTSSNTYNSRYWNNNPKPHRNTESFNHFSDLRQSFSGNAVIPSIYANGNHLVLGPHKTLVVTANRGSSPQVLPRNNGSVSRKPWPQHTHSYTVSQMTLERIGAVPVMVPAQSRAGSLV

SEQ ID NO: 379 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 380 - protein, 162 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAA

SEQ ID NO: 381 - protein, 136 aa, Homo sapiens
FKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQV

SEQ ID NO: 382 - protein, 303 aa, Homo sapiens
MGVPTAPEAGCWRWGSLLFALFLAASLGPVAAFKVATLYSLYVCPEGQNVTLTCRFFGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSSCVAYPSSSQESENITAAALATGACIVGILCLPLILLLVYKQRQAASNRRDNTQGIENPGFEASSPAQGILEAKVRHPLSYVAQRQPSESGRHLLSEPGTPLSPPGPGDVFFPSLDPVPDSPNFEVI

SEQ ID NO: 383 - protein, 308 aa, Homo sapiens
MGVPAVPEASSPRWGTLLLAIFLAASRGLVAAFKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITAAALATGACIVGILCLPLILLLVYKQRQVASHRRAQELVRMDSNTQGIENPGFETTPPFQGMPEAKTRPPLSYVAQRQPSESGRYLLSDPSTPLSPPGPGDVFFPSLDPVPDSPNSEAI

SEQ ID NO: 384 - RNA, 19 nt, Artificial
  siRNA
caacauaccauccauuuau

SEQ ID NO: 385 - RNA, 19 nt, Artificial
  siRNA
ggaacgaauuggugcagua

SEQ ID NO: 386 - RNA, 19 nt, Artificial
  siRNA
gaacaucagugcccugucu

SEQ ID NO: 387 - RNA, 19 nt, Artificial
  siRNA
caggaacauuggacuaaua

SEQ ID NO: 388 - protein, 105 aa, Homo sapiens
GLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIG

SEQ ID NO: 389 - protein, 121 aa, Homo sapiens
LEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVP

SEQ ID NO: 390 - protein, 98 aa, Homo sapiens
PSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIG

SEQ ID NO: 391 - protein, 5 aa, Homo sapiens
SYAMS

SEQ ID NO: 392 - protein, 17 aa, Homo sapiens
AISGSGGSTYYADSVKG

SEQ ID NO: 393 - protein, 7 aa, Homo sapiens
IHRPLDV

SEQ ID NO: 394 - protein, 116 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARIHRPLDVWGQGTLVTVSS

SEQ ID NO: 395 - protein, 13 aa, Homo sapiens
SGSSSNIGNNYVS

SEQ ID NO: 396 - protein, 7 aa, Homo sapiens
DNNKRPS

SEQ ID NO: 397 - protein, 11 aa, Homo sapiens
GSWLEERSQYV

SEQ ID NO: 398 - protein, 110 aa, Homo sapiens
ASVLTQPPSVSAAPGQKVTISCSGSSSNIGNNYVSWYQQLPGTAPKLLIYDNNKRPSGIPDRFSGSKSGTSATLGITGLQAEDEADYYCGSWLEERSQYVFGGGTKLTVL

SEQ ID NO: 399 - DNA, 348 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtctattattgtgcgcgcatccatcgtccactggatgtttggggccagggcaccctggttactgtctcgagc

SEQ ID NO: 400 - DNA, 330 bp, Homo sapiens
gcgagcgttctgacccagcctccgagcgttagcgcagcaccgggtcagaaagttaccattagctgtagcggtagcagcagcaatattggtaataactatgttagctggtatcagcagctgcctggcaccgcaccgaaactgctgatttatgataataacaaacgtccgagcggtattccggatcgttttagcggtagtaaaagcggcaccagcgcaaccctgggtattaccggtctgcaggcagaagacgaggctgattattattgcggttcttggctggaagaacgttctcagtacgtgttcggcggtggtaccaagttaaccgtgctg

SEQ ID NO: 401 - protein, 5 aa, Homo sapiens
SYAMS

SEQ ID NO: 402 - protein, 17 aa, Homo sapiens
AISGSGGSTYYADSVKG

SEQ ID NO: 403 - protein, 12 aa, Homo sapiens
DLSSGWGHAFDI

SEQ ID NO: 404 - protein, 121 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDLSSGWGHAFDIWGQGTMVPVSS

SEQ ID NO: 405 - protein, 13 aa, Homo sapiens
SGSSSNIGNNYVS

SEQ ID NO: 406 - protein, 7 aa, Homo sapiens
DNNKRPS

SEQ ID NO: 407 - protein, 11 aa, Homo sapiens
LSYTTSEHHLV

SEQ ID NO: 408 - protein, 110 aa, Homo sapiens
ASVLTQPPSVSAAPGQKVTISCSGSSSNIGNNYVSWYQQLPGTAPKLLIYDNNKRPSGIPDRFSGSKSGTSATLGITGLQAEDEADYYCLSYTTSEHHLVFGGGTKLTVL

SEQ ID NO: 409 - DNA, 363 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtgtattactgtgccagagacttaagtagtggttggggtcatgcttttgatatctggggccaggggacaatggtccccgtctcgagc

SEQ ID NO: 410 - DNA, 330 bp, Homo sapiens
gcgagcgttctgacccagcctccgagcgttagcgcagcaccgggtcagaaagttaccattagctgtagcggtagcagcagcaatattggtaataactatgttagctggtatcagcagctgcctggcaccgcaccgaaactgctgatttatgataataacaaacgtccgagcggtattccggatcgttttagcggtagtaaaagcggcaccagcgcaaccctgggtattaccggtctgcaggcagaagacgaggctgattattattgcctgtcttacactacttctgaacatcatctggtgttcggcggtggtaccaagttaaccgtgctg

SEQ ID NO: 411 - protein, 5 aa, Homo sapiens
SYAMS

SEQ ID NO: 412 - protein, 17 aa, Homo sapiens
AISGSGGSTYYADSVKG

SEQ ID NO: 413 - protein, 12 aa, Homo sapiens
DSRDAYGVAFDL

SEQ ID NO: 414 - protein, 121 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDSRDAYGVAFDLWGQGTMVTVSS

SEQ ID NO: 415 - protein, 13 aa, Homo sapiens
SGSSSNIGNNYVS

SEQ ID NO: 416 - protein, 7 aa, Homo sapiens
DNNKRPS

SEQ ID NO: 417 - protein, 9 aa, Homo sapiens
LSYTSSQYV

SEQ ID NO: 418 - protein, 108 aa, Homo sapiens
ASVLTQPPSVSAAPGQKVTISCSGSSSNIGNNYVSWYQQLPGTAPKLLIYDNNKRPSGIPDRFSGSKSGTSATLGITGLQAEDEADYYCLSYTSSQYVFGGGTKLTVL

SEQ ID NO: 419 - DNA, 363 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtatattactgtgcgagagactcaagagatgcctacggggttgcttttgatctctggggccaagggacaatggtcaccgtctcgagc

SEQ ID NO: 420 - DNA, 324 bp, Homo sapiens
gcgagcgttctgacccagcctccgagcgttagcgcagcaccgggtcagaaagttaccattagctgtagcggtagcagcagcaatattggtaataactatgttagctggtatcagcagctgcctggcaccgcaccgaaactgctgatttatgataataacaaacgtccgagcggtattccggatcgttttagcggtagtaaaagcggcaccagcgcaaccctgggtattaccggtctgcaggcagaagacgaggctgattattattgcctgtcttacacttcttctcagtacgtgttcggcggtggtaccaagttaaccgtgctg

SEQ ID NO: 421 - protein, 5 aa, Homo sapiens
SYAMS

SEQ ID NO: 422 - protein, 17 aa, Homo sapiens
AISGSGGSTYYADSVKG

SEQ ID NO: 423 - protein, 12 aa, Homo sapiens
DSRDAYGVAFDL

SEQ ID NO: 424 - protein, 121 aa, Homo sapiens; identical to SEQ ID NO: 414

SEQ ID NO: 425 - protein, 13 aa, Homo sapiens
SGSSSNIGNNYVS

SEQ ID NO: 426 - protein, 7 aa, Homo sapiens
DNNKRPS

SEQ ID NO: 427 - protein, 11 aa, Homo sapiens
LTWTGAGRIFV

SEQ ID NO: 428 - protein, 110 aa, Homo sapiens
ASVLTQPPSVSAAPGQKVTISCSGSSSNIGNNYVSWYQQLPGTAPKLLIYDNNKRPSGIPDRFSGSKSGTSATLGITGLQAEDEADYYCLTWTGAGRIFVFGGGTKLTVL

SEQ ID NO: 429 - DNA, 363 bp, Homo sapiens; identical to SEQ ID NO: 419

SEQ ID NO: 430 - DNA, 330 bp, Homo sapiens
gcgagcgttctgacccagcctccgagcgttagcgcagcaccgggtcagaaagttaccattagctgtagcggtagcagcagcaatattggtaataactatgttagctggtatcagcagctgcctggcaccgcaccgaaactgctgatttatgataataacaaacgtccgagcggtattccggatcgttttagcggtagtaaaagcggcaccagcgcaaccctgggtattaccggtctgcaggcagaagacgaggctgattattattgcctgacttggactggtgcaggtcgtatctttgtgttcggcggtggtaccaagttaaccgtgctg

SEQ ID NO: 431 - protein, 5 aa, Homo sapiens
NAWMS

SEQ ID NO: 432 - protein, 19 aa, Homo sapiens
RIKSKTDGGTTDYAAPVKG

SEQ ID NO: 433 - protein, 9 aa, Homo sapiens
LGIYSGFDY

SEQ ID NO: 434 - protein, 120 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCARLGIYSGFDYWGQGTLVTVSS

SEQ ID NO: 435 - protein, 11 aa, Homo sapiens
SGDKLGDKYAS

SEQ ID NO: 436 - protein, 7 aa, Homo sapiens
QDSKRPS

SEQ ID NO: 437 - protein, 11 aa, Homo sapiens
HSYTGKPSQVV

SEQ ID NO: 438 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCHSYTGKPSQVVFGGGTKLTVL

SEQ ID NO: 439 - DNA, 360 bp, Homo sapiens
gaagttcagctggttgaaagcggtggtggtctggttaaacctggtggtagcctgcgtctgagctgtgcagcaagcggttttacctttagcaatgcatggatgagctgggttcgtcaggcacctggtaaaggtctggaatgggttggtcgtattaaaagcaaaaccgatggtggcaccaccgattatgcagctccggttaaaggtcgttttaccattagtcgtgatgacagcaaaaataccctgtacctgcagatgaatagcctgaaaaccgaagatacggccgtctattattgtgcgcgcctgggtatctactctggttttgattactggggccagggcaccctggttactgtctcgagc

SEQ ID NO: 440 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgccattcttacactggtaaaccatctcaggttgtgttcggcggtggtaccaagttaaccgtgctg

SEQ ID NO: 441 - protein, 5 aa, Homo sapiens
SYAMS

SEQ ID NO: 442 - protein, 17 aa, Homo sapiens
AISGSGGSTYYADSVKG

SEQ ID NO: 443 - protein, 10 aa, Homo sapiens
HWVSYGPFDY

SEQ ID NO: 444 - protein, 119 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHWVSYGPFDYWGQGTLVTVSS

SEQ ID NO: 445 - protein, 11 aa, Homo sapiens
RASQSISSYLN

SEQ ID NO: 446 - protein, 7 aa, Homo sapiens
AASSLQS

SEQ ID NO: 447 - protein, 10 aa, Homo sapiens
QQSHQSPPIT

SEQ ID NO: 448 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSHQSPPITFGQGTKVEIK

SEQ ID NO: 449 - DNA, 357 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtatattactgtgcgagacattgggtcagctatggcccttttgactactggggccagggcaccctggtcaccgtctcgagc

SEQ ID NO: 450 - DNA, 324 bp, Homo sapiens
gatattcagatgacccagagtccgagcagcctgagcgcaagcgttggtgatcgtgttaccattacctgtcgtgcaagccagagcattagcagctatctgaattggtatcagcagaaaccgggtaaagcaccgaaactgctgatttatgcagcaagcagcctgcagagcggtgttccgagccgttttagcggatccggtagcggcaccgattttaccctgaccattagcagtctgcagccggaagactttgccacctattattgccagcagtctcatcagtctccgccgatcactttcggccagggtaccaaagtggaaattaag

SEQ ID NO: 451 - protein, 5 aa, Homo sapiens
SYAMS

SEQ ID NO: 452 - protein, 17 aa, Homo sapiens
AISGSGGSTYYADSVKG

SEQ ID NO: 453 - protein, 7 aa, Homo sapiens
IYRAFDY

SEQ ID NO: 454 - protein, 116 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARIYRAFDYWGQGTLVTVSS

SEQ ID NO: 455 - protein, 13 aa, Homo sapiens
SGSSSNIGNNYVS

SEQ ID NO: 456 - protein, 7 aa, Homo sapiens
DNNKRPS

SEQ ID NO: 457 - protein, 11 aa, Homo sapiens
QLYEEEHSTWV

SEQ ID NO: 458 - protein, 110 aa, Homo sapiens
ASVLTQPPSVSAAPGQKVTISCSGSSSNIGNNYVSWYQQLPGTAPKLLIYDNNKRPSGIPDRFSGSKSGTSATLGITGLQAEDEADYYCQLYEEEHSTWVFGGGTKLTVL

SEQ ID NO: 459 - DNA, 348 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtctattattgtgcgcgcatctaccgtgcatttgattactggggccagggcaccctggttactgtctcgagc

SEQ ID NO: 460 - DNA, 330 bp, Homo sapiens
gcgagcgttctgacccagcctccgagcgttagcgcagcaccgggtcagaaagttaccattagctgtagcggtagcagcagcaatattggtaataactatgttagctggtatcagcagctgcctggcaccgcaccgaaactgctgatttatgataataacaaacgtccgagcggtattccggatcgttttagcggtagtaaaagcggcaccagcgcaaccctgggtattaccggtctgcaggcagaagacgaggctgattattattgccagctgtacgaagaagaacattctacttgggtgttcggcggtggtaccaagttaaccgtgctg

SEQ ID NO: 461 - protein, 5 aa, Homo sapiens
SYAMS

SEQ ID NO: 462 - protein, 17 aa, Homo sapiens
AISGSGGSTYYADSVKG

SEQ ID NO: 463 - protein, 7 aa, Homo sapiens
DNGHNRD

SEQ ID NO: 464 - protein, 116 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCTTDNGHNRDWGQGTLVTVSS

SEQ ID NO: 465 - protein, 13 aa, Homo sapiens
SGSSSNIGNNYVS

SEQ ID NO: 466 - protein, 7 aa, Homo sapiens
DNNKRPS

SEQ ID NO: 467 - protein, 9 aa, Homo sapiens
GTWTRSSGV

SEQ ID NO: 468 - protein, 108 aa, Homo sapiens
ASVLTQPPSVSAAPGQKVTISCSGSSSNIGNNYVSWYQQLPGTAPKLLIYDNNKRPSGIPDRFSGSKSGTSATLGITGLQAEDEADYYCGTWTRSSGVFGGGTKLTVL

SEQ ID NO: 469 - DNA, 348 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtgtattactgtaccacagataatggccacaatagggactggggccagggcaccctggtcaccgtctcgagc

SEQ ID NO: 470 - DNA, 324 bp, Homo sapiens
gcgagcgttctgacccagcctccgagcgttagcgcagcaccgggtcagaaagttaccattagctgtagcggtagcagcagcaatattggtaataactatgttagctggtatcagcagctgcctggcaccgcaccgaaactgctgatttatgataataacaaacgtccgagcggtattccggatcgttttagcggtagtaaaagcggcaccagcgcaaccctgggtattaccggtctgcaggcagaagacgaggctgattattattgcggtacttggactcgttcttctggtgtgttcggcggtggtaccaagttaaccgtgctg

SEQ ID NO: 471 - protein, 5 aa, Homo sapiens
SYAMS

SEQ ID NO: 472 - protein, 17 aa, Homo sapiens
AISGSGGSTYYADSVKG

SEQ ID NO: 473 - protein, 14 aa, Homo sapiens
GHHHGVYYFYAMDL

SEQ ID NO: 474 - protein, 123 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGHHHGVYYFYAMDLWGQGTLVTVSS

SEQ ID NO: 475 - protein, 11 aa, Homo sapiens
SGDKLGDKYAS

SEQ ID NO: 476 - protein, 7 aa, Homo sapiens
QDSKRPS

SEQ ID NO: 477 - protein, 11 aa, Homo sapiens
QSYSGSSTLHV

SEQ ID NO: 478 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCQSYSGSSTLHVFGGGTKLTVL

SEQ ID NO: 479 - DNA, 369 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtctattattgtgcgcgcggtcatcatcatggtgtttactacttttacgcaatggatctgtggggccagggcaccctggttactgtctcgagc

SEQ ID NO: 480 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgccagtcttactctggttcttctactctgcatgtgttcggcggtggtaccaagttaaccgtgctg

SEQ ID NO: 481 - protein, 5 aa, Homo sapiens
SYAIS

SEQ ID NO: 482 - protein, 17 aa, Homo sapiens
GIIPIFGTANYAQKFQG

SEQ ID NO: 483 - protein, 11 aa, Homo sapiens
GYGEYYPAFDV

SEQ ID NO: 484 - protein, 120 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYGEYYPAFDVWGQGTLVTVSS

SEQ ID NO: 485 - protein, 11 aa, Homo sapiens
SGDKLGDKYAS

SEQ ID NO: 486 - protein, 7 aa, Homo sapiens
QDSKRPS

SEQ ID NO: 487 - protein, 11 aa, Homo sapiens
ASYAHTHSTWV

SEQ ID NO: 488 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCASYAHTHSTWVFGGGTKLTVL

SEQ ID NO: 489 - DNA, 360 bp, Homo sapiens
caggttcagctggttcagagcggtgcagaagttaaaaaaccgggtagcagcgttaaagttagctgtaaagcaagcggtggcacctttagcagctatgcaattagctgggttcgtcaggcacctggtcaaggtctggaatggatgggtggtattattccgatttttggcaccgcaaattatgcccagaaatttcagggtcgtgttaccattaccgcagatgaaagcaccagcaccgcatatatggaactgagcagcctgcgtagcgaagatacggccgtctattattgtgcgcgcggttacggtgaatactacccagcatttgatgtttggggccagggcaccctggttactgtctcgagc

SEQ ID NO: 490 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgcgcatcttacgcacatactcattctacttgggtgttcggcggtggtaccaagttaaccgtgctg

SEQ ID NO: 491 - protein, 5 aa, Homo sapiens
SYAIS

SEQ ID NO: 492 - protein, 17 aa, Homo sapiens
GIIPIFGTANYAQKFQG

SEQ ID NO: 493 - protein, 9 aa, Homo sapiens
HSTPSFLQY

SEQ ID NO: 494 - protein, 118 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARHSTPSFLQYWGQGTLVTVSS

SEQ ID NO: 495 - protein, 11 aa, Homo sapiens
SGDKLGDKYAS

SEQ ID NO: 496 - protein, 7 aa, Homo sapiens
QDSKRPS

SEQ ID NO: 497 - protein, 12 aa, Homo sapiens
AVYPAHASARWV

SEQ ID NO: 498 - protein, 109 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCAVYPAHASARWVFGGGTKLTVL

SEQ ID NO: 499 - DNA, 354 bp, Homo sapiens
caggttcagctggttcagagcggtgcagaagttaaaaaaccgggtagcagcgttaaagttagctgtaaagcaagcggtggcacctttagcagctatgcaattagctgggttcgtcaggcacctggtcaaggtctggaatggatgggtggtattattccgatttttggcaccgcaaattatgcccagaaatttcagggtcgtgttaccattaccgcagatgaaagcaccagcaccgcatatatggaactgagcagcctgcgtagcgaagatacggccgtctattattgtgcgcgccattctactccatcttttctgcagtactggggccagggcaccctggttactgtctcgagc

SEQ ID NO: 500 - DNA, 327 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgcgcagtttacccagcacatgcatctgcacgttgggtgttcggcggtggtaccaagttaaccgtgctg

SEQ ID NO: 501 - protein, 5 aa, Homo sapiens
SYAMS

SEQ ID NO: 502 - protein, 17 aa, Homo sapiens
AISGSGGSTYYADSVKG

SEQ ID NO: 503 - protein, 10 aa, Homo sapiens
HWVSYGPFDY

SEQ ID NO: 504 - protein, 119 aa, Homo sapiens; identical to SEQ ID NO: 444

SEQ ID NO: 505 - protein, 11 aa, Homo sapiens
RASQSISSYLN

SEQ ID NO: 506 - protein, 7 aa, Homo sapiens
AASSLQS

SEQ ID NO: 507 - protein, 10 aa, Homo sapiens
QQSHQSPPIT

SEQ ID NO: 508 - protein, 108 aa, Homo sapiens; identical to SEQ ID NO: 448

SEQ ID NO: 509 - DNA, 357 bp, Homo sapiens; identical to SEQ ID NO: 449

SEQ ID NO: 510 - DNA, 324 bp, Homo sapiens; identical to SEQ ID NO: 450

SEQ ID NO: 511 - protein, 5 aa, Homo sapiens
NAWMS

SEQ ID NO: 512 - protein, 19 aa, Homo sapiens
RIKSKTDGGTTDYAAPVKG

SEQ ID NO: 513 - protein, 9 aa, Homo sapiens
IEGSHGFDY

SEQ ID NO: 514 - protein, 120 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCARIEGSHGFDYWGQGTLVTVSS

SEQ ID NO: 515 - protein, 11 aa, Homo sapiens
SGDKLGDKYAS

SEQ ID NO: 516 - protein, 7 aa, Homo sapiens
QDSKRPS

SEQ ID NO: 517 - protein, 11 aa, Homo sapiens
ASYLHTPKQFV

SEQ ID NO: 518 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCASYLHTPKQFVFGGGTKLTVL

SEQ ID NO: 519 - DNA, 360 bp, Homo sapiens
gaagttcagctggttgaaagcggtggtggtctggttaaacctggtggtagcctgcgtctgagctgtgcagcaagcggttttacctttagcaatgcatggatgagctgggttcgtcaggcacctggtaaaggtctggaatgggttggtcgtattaaaagcaaaaccgatggtggcaccaccgattatgcagctccggttaaaggtcgttttaccattagtcgtgatgacagcaaaaataccctgtacctgcagatgaatagcctgaaaaccgaagatacggccgtctattattgtgcgcgcatcgaaggttctcatggttttgattactggggccagggcaccctggttactgtctcgagc

SEQ ID NO: 520 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgcgcatcttacctgcatactccaaaacagtttgtgttcggcggtggtaccaagttaaccgtgctg

SEQ ID NO: 521 - protein, 5 aa, Homo sapiens
NAWMS

SEQ ID NO: 522 - protein, 19 aa, Homo sapiens
RIKSKTDGGTTDYAAPVKG

SEQ ID NO: 523 - protein, 9 aa, Homo sapiens
LGSYEGFDY

SEQ ID NO: 524 - protein, 120 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCARLGSYEGFDYWGQGTLVTVSS

SEQ ID NO: 525 - protein, 11 aa, Homo sapiens
SGDKLGDKYAS

SEQ ID NO: 526 - protein, 7 aa, Homo sapiens
QDSKRPS

SEQ ID NO: 527 - protein, 11 aa, Homo sapiens
STYTVTSSVVV

SEQ ID NO: 528 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCSTYTVTSSVVVFGGGTKLTVL

SEQ ID NO: 529 - DNA, 360 bp, Homo sapiens
gaagttcagctggttgaaagcggtggtggtctggttaaacctggtggtagcctgcgtctgagctgtgcagcaagcggttttacctttagcaatgcatggatgagctgggttcgtcaggcacctggtaaaggtctggaatgggttggtcgtattaaaagcaaaaccgatggtggcaccaccgattatgcagctccggttaaaggtcgttttaccattagtcgtgatgacagcaaaaataccctgtacctgcagatgaatagcctgaaaaccgaagatacggccgtctattattgtgcgcgcctgggttcttacgaaggttttgattactggggccagggcaccctggttactgtctcgagc

SEQ ID NO: 530 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgctctacttacactgttacttcttctgttgttgtgttcggcggtggtaccaagttaaccgtgctg

SEQ ID NO: 531 - protein, 5 aa, Homo sapiens
NAWMS

SEQ ID NO: 532 - protein, 19 aa, Homo sapiens
RIKSKTDGGTTDYAAPVKG

SEQ ID NO: 533 - protein, 12 aa, Homo sapiens
VAHGGYSGGLDP

SEQ ID NO: 534 - protein, 123 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCARVAHGGYSGGLDPWGQGTLVTVSS

SEQ ID NO: 535 - protein, 11 aa, Homo sapiens
SGDKLGDKYAS

SEQ ID NO: 536 - protein, 7 aa, Homo sapiens
QDSKRPS

SEQ ID NO: 537 - protein, 11 aa, Homo sapiens
SSTGTAHTLAV

SEQ ID NO: 538 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCSSTGTAHTLAVFGGGTKLTVL

SEQ ID NO: 539 - DNA, 369 bp, Homo sapiens
gaagttcagctggttgaaagcggtggtggtctggttaaacctggtggtagcctgcgtctgagctgtgcagcaagcggttttacctttagcaatgcatggatgagctgggttcgtcaggcacctggtaaaggtctggaatgggttggtcgtattaaaagcaaaaccgatggtggcaccaccgattatgcagctccggttaaaggtcgttttaccattagtcgtgatgacagcaaaaataccctgtacctgcagatgaatagcctgaaaaccgaagatacggccgtctattattgtgcgcgcgttgcacatggtggttactctggtggtctggatccatggggccagggcaccctggttactgtctcgagc

SEQ ID NO: 540 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgctcttctactggtactgcacatactctggcagtgttcggcggtggtaccaagttaaccgtgctg

SEQ ID NO: 541 - protein, 5 aa, Homo sapiens
SYAMS

SEQ ID NO: 542 - protein, 17 aa, Homo sapiens
AISGSGGSTYYADSVKG

SEQ ID NO: 543 - protein, 7 aa, Homo sapiens
VYRAFDY

SEQ ID NO: 544 - protein, 116 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARVYRAFDYWGQGTLVTVSS

SEQ ID NO: 545 - protein, 13 aa, Homo sapiens
SGSSSNIGNNYVS

SEQ ID NO: 546 - protein, 7 aa, Homo sapiens
DNNKRPS

SEQ ID NO: 547 - protein, 11 aa, Homo sapiens
HLYTEAESHWV

SEQ ID NO: 548 - protein, 110 aa, Homo sapiens
ASVLTQPPSVSAAPGQKVTISCSGSSSNIGNNYVSWYQQLPGTAPKLLIYDNNKRPSGIPDRFSGSKSGTSATLGITGLQAEDEADYYCHLYTEAESHWVFGGGTKLTVL

SEQ ID NO: 549 - DNA, 348 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtctattattgtgcgcgcgtttaccgtgcatttgattactggggccagggcaccctggttactgtctcgagc

SEQ ID NO: 550 - DNA, 330 bp, Homo sapiens
gcgagcgttctgacccagcctccgagcgttagcgcagcaccgggtcagaaagttaccattagctgtagcggtagcagcagcaatattggtaataactatgttagctggtatcagcagctgcctggcaccgcaccgaaactgctgatttatgataataacaaacgtccgagcggtattccggatcgttttagcggtagtaaaagcggcaccagcgcaaccctgggtattaccggtctgcaggcagaagacgaggctgattattattgccatctgtacactgaagcagaatctcattgggtgttcggcggtggtaccaagttaaccgtgctg

SEQ ID NO: 551 - protein, 5 aa, Homo sapiens
SYAMS

SEQ ID NO: 552 - protein, 17 aa, Homo sapiens
AISGSGGSTYYADSVKG

SEQ ID NO: 553 - protein, 9 aa, Homo sapiens
DGSGPTLDL

SEQ ID NO: 554 - protein, 118 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARDGSGPTLDLWGRGTLVTVSS

SEQ ID NO: 555 - protein, 11 aa, Homo sapiens
RASQSISSYLN

SEQ ID NO: 556 - protein, 7 aa, Homo sapiens
AASSLQS

SEQ ID NO: 557 - protein, 10 aa, Homo sapiens
QQHRYIPPWT

SEQ ID NO: 558 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQHRYIPPWTFGQGTKVEIK

SEQ ID NO: 559 - DNA, 354 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtgtattactgtgcgagagatggttccggccccactctcgatctctggggccgtggcaccctggtcactgtctcgagc

SEQ ID NO: 560 - DNA, 324 bp, Homo sapiens
gatattcagatgacccagagtccgagcagcctgagcgcaagcgttggtgatcgtgttaccattacctgtcgtgcaagccagagcattagcagctatctgaattggtatcagcagaaaccgggtaaagcaccgaaactgctgatttatgcagcaagcagcctgcagagcggtgttccgagccgttttagcggatccggtagcggcaccgattttaccctgaccattagcagtctgcagccggaagactttgccacctattattgccagcagcatcgttacatcccgccgtggactttcggccagggtaccaaagtggaaattaag

SEQ ID NO: 561 - protein, 5 aa, Homo sapiens
SYAMS

SEQ ID NO: 562 - protein, 17 aa, Homo sapiens
AISGSGGSTYYADSVKG

SEQ ID NO: 563 - protein, 14 aa, Homo sapiens
STPGYYYVHYGFDI

SEQ ID NO: 564 - protein, 123 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARSTPGYYYVHYGFDIWGQGTLVTVSS

SEQ ID NO: 565 - protein, 11 aa, Homo sapiens
SGDKLGDKYAS

SEQ ID NO: 566 - protein, 7 aa, Homo sapiens
QDSKRPS

SEQ ID NO: 567 - protein, 11 aa, Homo sapiens
QAWSTSTHSWV

SEQ ID NO: 568 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCQAWSTSTHSWVFGGGTKLTVL

SEQ ID NO: 569 - DNA, 369 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtctattattgtgcgcgctctactccaggttactactacgttcattacggttttgatatctggggccagggcaccctggttactgtctcgagc

SEQ ID NO: 570 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgccaggcatggtctacttctactcattcttgggtgttcggcggtggtaccaagttaaccgtgctg

SEQ ID NO: 571 - protein, 5 aa, Homo sapiens
SYAMS

SEQ ID NO: 572 - protein, 17 aa, Homo sapiens
AISGSGGSTYYADSVKG

SEQ ID NO: 573 - protein, 11 aa, Homo sapiens
GSPYVVGVFDY

SEQ ID NO: 574 - protein, 120 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGSPYVVGVFDYWGQGTLVTVSS

SEQ ID NO: 575 - protein, 11 aa, Homo sapiens
RASQSISSYLN

SEQ ID NO: 576 - protein, 7 aa, Homo sapiens
AASSLQS

SEQ ID NO: 577 - protein, 10 aa, Homo sapiens
QQWQHEPPYT

SEQ ID NO: 578 - protein, 108 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQWQHEPPYTFGQGTKVEIK

SEQ ID NO: 579 - DNA, 360 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtctattattgtgcgcgcggttctccatacgttgttggtgtttttgattactggggccagggcaccctggttactgtctcgagc

SEQ ID NO: 580 - DNA, 324 bp, Homo sapiens
gatattcagatgacccagagtccgagcagcctgagcgcaagcgttggtgatcgtgttaccattacctgtcgtgcaagccagagcattagcagctatctgaattggtatcagcagaaaccgggtaaagcaccgaaactgctgatttatgcagcaagcagcctgcagagcggtgttccgagccgttttagcggatccggtagcggcaccgattttaccctgaccattagcagtctgcagccggaagactttgccacctattattgccagcagtggcagcatgaaccgccgtacactttcggccagggtaccaaagtggaaattaag

SEQ ID NO: 581 - protein, 5 aa, Homo sapiens
NAWMS

SEQ ID NO: 582 - protein, 19 aa, Homo sapiens
RIKSKTDGGTTDYAAPVKG

SEQ ID NO: 583 - protein, 9 aa, Homo sapiens
LHIYGPFDY

SEQ ID NO: 584 - protein, 120 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCARLHIYGPFDYWGQGTLVTVSS

SEQ ID NO: 585 - protein, 11 aa, Homo sapiens
SGDKLGDKYAS

SEQ ID NO: 586 - protein, 7 aa, Homo sapiens
QDSKRPS

SEQ ID NO: 587 - protein, 11 aa, Homo sapiens
LSYLARSGSVV

SEQ ID NO: 588 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCLSYLARSGSVVFGGGTKLTVL

SEQ ID NO: 589 - DNA, 360 bp, Homo sapiens
gaagttcagctggttgaaagcggtggtggtctggttaaacctggtggtagcctgcgtctgagctgtgcagcaagcggttttacctttagcaatgcatggatgagctgggttcgtcaggcacctggtaaaggtctggaatgggttggtcgtattaaaagcaaaaccgatggtggcaccaccgattatgcagctccggttaaaggtcgttttaccattagtcgtgatgacagcaaaaataccctgtacctgcagatgaatagcctgaaaaccgaagatacggccgtctattattgtgcgcgcctgcatatctacggtccatttgattactggggccagggcaccctggttactgtctcgagc

SEQ ID NO: 590 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgcctgtcttacctggcacgttctggttctgttgtgttcggcggtggtaccaagttaaccgtgctg

SEQ ID NO: 591 - protein, 5 aa, Homo sapiens
SYAMS

SEQ ID NO: 592 - protein, 17 aa, Homo sapiens
AISGSGGSTYYADSVKG

SEQ ID NO: 593 - protein, 17 aa, Homo sapiens
HEVFGTSSGYHLYAFDI

SEQ ID NO: 594 - protein, 126 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHEVFGTSSGYHLYAFDIWGQGTLVTVSS

SEQ ID NO: 595 - protein, 11 aa, Homo sapiens
RASQSISSYLN

SEQ ID NO: 596 - protein, 7 aa, Homo sapiens
AASSLQS

SEQ ID NO: 597 - protein, 9 aa, Homo sapiens
QQWSGLPLT

SEQ ID NO: 598 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQWSGLPLTFGQGTKVEIK

SEQ ID NO: 599 - DNA, 378 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtctattattgtgcgcgccatgaagtttttggtacttcttctggttaccatctgtacgcatttgatatctggggccagggcaccctggttactgtctcgagc

SEQ ID NO: 600 - DNA, 321 bp, Homo sapiens
gatattcagatgacccagagtccgagcagcctgagcgcaagcgttggtgatcgtgttaccattacctgtcgtgcaagccagagcattagcagctatctgaattggtatcagcagaaaccgggtaaagcaccgaaactgctgatttatgcagcaagcagcctgcagagcggtgttccgagccgttttagcggatccggtagcggcaccgattttaccctgaccattagcagtctgcagccggaagactttgccacctattattgccagcagtggtctggtctgccgctgactttcggccagggtaccaaagtggaaattaag

SEQ ID NO: 601 - protein, 5 aa, Homo sapiens
SYAMS

SEQ ID NO: 602 - protein, 17 aa, Homo sapiens
AISGSGGSTYYADSVKG

SEQ ID NO: 603 - protein, 12 aa, Homo sapiens
MAAGASWGTFDY

SEQ ID NO: 604 - protein, 121 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAAYYCARMAAGASWGTFDYWSQGTLVTVSS

SEQ ID NO: 605 - protein, 11 aa, Homo sapiens
RASQSISSYLN

SEQ ID NO: 606 - protein, 7 aa, Homo sapiens
AASSLQS

SEQ ID NO: 607 - protein, 9 aa, Homo sapiens
QQRSGSPLT

SEQ ID NO: 608 - protein, 107 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQRSGSPLTFGQGTKVEIK

SEQ ID NO: 609 - DNA, 363 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgcatattactgtgcacggatggctgcaggcgccagttgggggaccttcgactactggagccagggaaccctggtcaccgtctcgagc

SEQ ID NO: 610 - DNA, 321 bp, Homo sapiens
gatattcagatgacccagagtccgagcagcctgagcgcaagcgttggtgatcgtgttaccattacctgtcgtgcaagccagagcattagcagctatctgaattggtatcagcagaaaccgggtaaagcaccgaaactgctgatttatgcagcaagcagcctgcagagcggtgttccgagccgttttagcggatccggtagcggcaccgattttaccctgaccattagcagtctgcagccggaagactttgccacctattattgccagcagcgttctggttctccgctgactttcggccagggtaccaaagtggaaattaag

SEQ ID NO: 611 - protein, 5 aa, Homo sapiens
NAWMS

SEQ ID NO: 612 - protein, 19 aa, Homo sapiens
RIKSKTDGGTTDYAAPVKG

SEQ ID NO: 613 - protein, 9 aa, Homo sapiens
LGVFSGFDY

SEQ ID NO: 614 - protein, 120 aa, Homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCARLGVFSGFDYWGQGTLVTVSS

SEQ ID NO: 615 - protein, 11 aa, Homo sapiens
SGDKLGDKYAS

SEQ ID NO: 616 - protein, 7 aa, Homo sapiens
QDSKRPS

SEQ ID NO: 617 - protein, 11 aa, Homo sapiens
HTWTHHSLAVV

SEQ ID NO: 618 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCHTWTHHSLAVVFGGGTKLTVL

SEQ ID NO: 619 - DNA, 360 bp, Homo sapiens
gaagttcagctggttgaaagcggtggtggtctggttaaacctggtggtagcctgcgtctgagctgtgcagcaagcggttttacctttagcaatgcatggatgagctgggttcgtcaggcacctggtaaaggtctggaatgggttggtcgtattaaaagcaaaaccgatggtggcaccaccgattatgcagctccggttaaaggtcgttttaccattagtcgtgatgacagcaaaaataccctgtacctgcagatgaatagcctgaaaaccgaagatacggccgtctattattgtgcgcgcctgggtgttttttctggttttgattactggggccagggcaccctggttactgtctcgagc

SEQ ID NO: 620 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgccatacttggactcatcattctctggcagttgtgttcggcggtggtaccaagttaaccgtgctg

SEQ ID NO: 621 - protein, 5 aa, Homo sapiens
SYAMS

SEQ ID NO: 622 - protein, 17 aa, Homo sapiens
AISGSGGSTYYADSVKG

SEQ ID NO: 623 - protein, 10 aa, Homo sapiens
HEYLGFYFDV

SEQ ID NO: 624 - protein, 119 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHEYLGFYFDVWGQGTLVTVSS

SEQ ID NO: 625 - protein, 11 aa, Homo sapiens
SGDKLGDKYAS

SEQ ID NO: 626 - protein, 7 aa, Homo sapiens
QDSKRPS

SEQ ID NO: 627 - protein, 11 aa, Homo sapiens
LASHRLTLNYV

SEQ ID NO: 628 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCLASHRLTLNYVFGGGTKLTVL

SEQ ID NO: 629 - DNA, 357 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtctattattgtgcgcgccatgaatacctgggtttttactttgatgtttggggccagggcaccctggttactgtctcgagc

SEQ ID NO: 630 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgcctggcatctcatcgtctgactctgaactacgtgttcggcggtggtaccaagttaaccgtgctg

SEQ ID NO: 631 - protein, 5 aa, Homo sapiens
SYAMS

SEQ ID NO: 632 - protein, 17 aa, Homo sapiens
AISGSGGSTYYADSVKG

SEQ ID NO: 633 - protein, 14 aa, Homo sapiens
HYTVGVYVYEYFDY

SEQ ID NO: 634 - protein, 123 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHYTVGVYVYEYFDYWGQGTLVTVSS

SEQ ID NO: 635 - protein, 11 aa, Homo sapiens
SGDKLGDKYAS

SEQ ID NO: 636 - protein, 7 aa, Homo sapiens
QDSKRPS

SEQ ID NO: 637 - protein, 9 aa, Homo sapiens
QSYATAGFV

SEQ ID NO: 638 - protein, 106 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCQSYATAGFVFGGGTKLTVL

SEQ ID NO: 639 - DNA, 369 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtctattattgtgcgcgccattacactgttggtgtttacgtttacgaatactttgattactggggccagggcaccctggttactgtctcgagc

SEQ ID NO: 640 - DNA, 318 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgccagtcttacgcaactgcaggttttgtgttcggcggtggtaccaagttaaccgtgctg

SEQ ID NO: 641 - protein, 5 aa, Homo sapiens
SYAMS

SEQ ID NO: 642 - protein, 17 aa, Homo sapiens
AISGSGGSTYYADSVKG

SEQ ID NO: 643 - protein, 16 aa, Homo sapiens
LHKVFEFYHYTYAFDY

SEQ ID NO: 644 - protein, 125 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARLHKVFEFYHYTYAFDYWGQGTLVTVSS

SEQ ID NO: 645 - protein, 13 aa, Homo sapiens
SGSSSNIGNNYVS

SEQ ID NO: 646 - protein, 7 aa, Homo sapiens
DNNKRPS

SEQ ID NO: 647 - protein, 11 aa, Homo sapiens
ASYSSETSGWV

SEQ ID NO: 648 - protein, 110 aa, Homo sapiens
ASVLTQPPSVSAAPGQKVTISCSGSSSNIGNNYVSWYQQLPGTAPKLLIYDNNKRPSGIPDRFSGSKSGTSATLGITGLQAEDEADYYCASYSSETSGWVFGGGTKLTVL

SEQ ID NO: 649 - DNA, 375 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtctattattgtgcgcgcctgcataaagtttttgaattttaccattacacttacgcatttgattactggggccagggcaccctggttactgtctcgagc

SEQ ID NO: 650 - DNA, 330 bp, Homo sapiens
gcgagcgttctgacccagcctccgagcgttagcgcagcaccgggtcagaaagttaccattagctgtagcggtagcagcagcaatattggtaataactatgttagctggtatcagcagctgcctggcaccgcaccgaaactgctgatttatgataataacaaacgtccgagcggtattccggatcgttttagcggtagtaaaagcggcaccagcgcaaccctgggtattaccggtctgcaggcagaagacgaggctgattattattgcgcatcttactcttctgaaacttctggttgggtgttcggcggcggtaccaagttaaccgtgctg

SEQ ID NO: 651 - protein, 5 aa, Homo sapiens
SYAIS

SEQ ID NO: 652 - protein, 17 aa, Homo sapiens
GIIPIFGTANYAQKFQG

SEQ ID NO: 653 - protein, 18 aa, Homo sapiens
ENIPSYYDSSGRQDAFDI

SEQ ID NO: 654 - protein, 127 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARENIPSYYDSSGRQDAFDIWGQGTMVTVSS

SEQ ID NO: 655 - protein, 11 aa, Homo sapiens
SGDKLGDKYAS

SEQ ID NO: 656 - protein, 7 aa, Homo sapiens
QDSKRPS

SEQ ID NO: 657 - protein, 11 aa, Homo sapiens
QSYLHKSHGAV

SEQ ID NO: 658 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCQSYLHKSHGAVFGGGTKLTVL

SEQ ID NO: 659 - DNA, 381 bp, Homo sapiens
caggttcagctggttcagagcggtgcagaagttaaaaaaccgggtagcagcgttaaagttagctgtaaagcaagcggtggcacctttagcagctatgcaattagctgggttcgtcaggcacctggtcaaggtctggaatggatgggtggtattattccgatttttggcaccgcaaattatgcccagaaatttcagggtcgtgttaccattaccgcagatgaaagcaccagcaccgcatatatggaactgagcagcctgcgtagcgaagatacggccgtatattactgtgcgagagaaaatatccctagttactatgatagtagtggccgccaggatgcttttgatatctggggccaagggacaatggtcaccgtctcgagc

SEQ ID NO: 660 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgccagtcttacctgcataaatctcatggtgcagtgttcggcggtggtaccaagttaaccgtgctg

SEQ ID NO: 661 - protein, 5 aa, Homo sapiens
SYAMS

SEQ ID NO: 662 - protein, 17 aa, Homo sapiens
AISGSGGSTYYADSVKG

SEQ ID NO: 663 - protein, 18 aa, Homo sapiens
GQYVSGTYYSYGYWYFDL

SEQ ID NO: 664 - protein, 127 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGQYVSGTYYSYGYWYFDLWGRGTLVTVSS

SEQ ID NO: 665 - protein, 11 aa, Homo sapiens
SGDKLGDKYAS

SEQ ID NO: 666 - protein, 7 aa, Homo sapiens
QDSKRPS

SEQ ID NO: 667 - protein, 11 aa, Homo sapiens
QVSRGHTSAGV

SEQ ID NO: 668 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCQVSRGHTSAGVFGGGTKLTVL

SEQ ID NO: 669 - DNA, 381 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtgtattactgtgcgagagggcaatatgtgtcggggacttattattcctacggatactggtacttcgatctctggggccgtggcaccctggtcactgtctcgagc

SEQ ID NO: 670 - DNA, 324 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgccaggtttctcgtggtcatacttctgcaggtgtgttcggcggtggtaccaagttaaccgtgctg

SEQ ID NO: 671 - protein, 5 aa, Homo sapiens
SYAMS

SEQ ID NO: 672 - protein, 17 aa, Homo sapiens
AISGSGGSTYYADSVKG

SEQ ID NO: 673 - protein, 14 aa, Homo sapiens
HHGHGIYVHYYLDY

SEQ ID NO: 674 - protein, 123 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARHHGHGIYVHYYLDYWGQGTLVTVSS

SEQ ID NO: 675 - protein, 11 aa, Homo sapiens
SGDKLGDKYAS

SEQ ID NO: 676 - protein, 7 aa, Homo sapiens
QDSKRPS

SEQ ID NO: 677 - protein, 10 aa, Homo sapiens
QTWAGTRLVV

SEQ ID NO: 678 - protein, 107 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCQTWAGTRLVVFGGGTKLTVL

SEQ ID NO: 679 - DNA, 369 bp, Homo sapiens
gaagttcagctgctggaaagcggtggtggtctggttcagcctggtggtagcctgcgtctgagctgtgcagcaagcggttttacctttagcagctatgcaatgagctgggttcgtcaggcacctggtaaaggtctggaatgggttagcgcaattagcggtagcggtggtagcacctattatgcagatagcgttaaaggtcgctttaccattagccgtgataatagcaaaaataccctgtacctgcagatgaatagtctgcgtgcagaagatacggccgtctattattgtgcgcgccatcatggtcatggtatctacgttcattactacctggattactggggccagggcaccctggttactgtctcgagc

SEQ ID NO: 680 - DNA, 321 bp, Homo sapiens
agctatgaactgacccagcctccgagcgttagcgttagtccgggtcagaccgcaagcattacctgtagcggtgataaactgggtgataaatatgcaagctggtatcagcagaaaccgggtcagtcaccggttctggttatttatcaggatagcaaacgtccgagcggtattccggaacgttttagcggatccaatagtggtaataccgcaacactgaccattagcggcacccaggctgaagacgaggctgattattattgccagacttgggcaggtactcgtctggttgtgttcggcggtggtaccaagttaaccgtgctg

SEQ ID NO: 681 - protein, 5 aa, Homo sapiens
FSALS

SEQ ID NO: 682 - protein, 17 aa, Homo sapiens
AISYGGGSKYYADSVKG

SEQ ID NO: 683 - protein, 12 aa, Homo sapiens
DSRDAYGVAFDL

SEQ ID NO: 684 - protein, 121 aa, Homo sapiens
EVQLLESGGGLVQPGGSLRLSCAASGFTFQFSALSWVRQAPGKGLEWVSAISYGGGSKYYADSVKGRFTISRDNSKNTLYLQMNSLRAEDTAVYYCARDSRDAYGVAFDLWGQGTMVTVSS

SEQ ID NO: 685 - protein, 13 aa, Homo sapiens
SGSSSNIGNNYVS

SEQ ID NO: 686 - protein, 7 aa, Homo sapiens
DNNKRPS

SEQ ID NO: 687 - protein, 11 aa, Homo sapiens
LTWTGAGRIFV

SEQ ID NO: 688 - protein, 110 aa, Homo sapiens; identical to SEQ ID NO: 428

SEQ ID NO: 689 - DNA, 363 bp, Homo sapiens
gaagtgcagctgcttgaatctggcggaggactggttcagcctggcggatctctgagactgtcttgtgccgccagcggcttcaccttccagttttctgccctgagctgggtccgacaggcccctggaaaaggactggaatgggtgtccgccatctcttacggcggaggcagcaagtactacgccgactctgtgaagggcagattcaccatcagccgggacaacagcaagaacaccctgtacctgcagatgaacagcctgagagccgaggacaccgccgtgtactactgtgccagagacagcagagatgcctacggcgtggcctttgatctgtggggccagggcacaatggtcacagtctcgagc

SEQ ID NO: 690 - DNA, 330 bp, Homo sapiens; identical to SEQ ID NO: 430

SEQ ID NO: 691 - protein, 5 aa, Homo sapiens
KYSLS

SEQ ID NO: 692 - protein, 17 aa, Homo sapiens
AISYYGGGTLYADSVKG

SEQ ID NO: 693 - protein, 12 aa, Homo sapiens
DSRDAYGVAFDL

SEQ ID NO: 694 - protein, 121 aa, Homo sapiens
EVQLLESGGGLVQPGGSLRLSCAASGFTFSKYSLSWVRQAPGKGLEWVSAISYYGGGTLYADSVKGRFTISRDNSKNTLYLQMNSLRAEDTAVYYCARDSRDAYGVAFDLWGQGTMVTVSS

SEQ ID NO: 695 - protein, 13 aa, Homo sapiens
SGSSSNIGNNYVS

SEQ ID NO: 696 - protein, 7 aa, Homo sapiens
DNNKRPS

SEQ ID NO: 697 - protein, 11 aa, Homo sapiens
LTWTGAGRIFV

SEQ ID NO: 698 - protein, 110 aa, Homo sapiens; identical to SEQ ID NO: 428

SEQ ID NO: 699 - DNA, 363 bp, Homo sapiens
gaagtgcagctgcttgaatctggcggaggactggttcaacctggcggctctctgagactgtcttgtgccgccagcggcttcacctttagcaagtattctctgagctgggtccgacaggcccctggaaaaggacttgaatgggtgtccgccatcagctactatggcggcggaacactttacgccgatagcgtgaagggcagattcaccatcagccgggacaacagcaagaacaccctgtacctgcagatgaacagcctgagagccgaggacaccgccgtgtactactgtgccagagacagcagagatgcctacggcgtggcatttgatctgtggggccagggcacaatggtcacagtctcgagc

SEQ ID NO: 700 - DNA, 330 bp, Homo sapiens; identical to SEQ ID NO: 430

SEQ ID NO: 701 - protein, 5 aa, Homo sapiens
HSAIS

SEQ ID NO: 702 - protein, 17 aa, Homo sapiens
AISYGGGSQYYADSVKG

SEQ ID NO: 703 - protein, 12 aa, Homo sapiens
DSRDAYGVAFDL

SEQ ID NO: 704 - protein, 121 aa, Homo sapiens
EVQLLESGGGLVQPGGSLRLSCAASGFTFKHSAISWVRQAPGKGLEWVSAISYGGGSQYYADSVKGRFTISRDNSKNTLYLQMNSLRAEDTAVYYCARDSRDAYGVAFDLWGQGTMVTVSS

SEQ ID NO: 705 - protein, 13 aa, Homo sapiens
SGSSSNIGNNYVS

SEQ ID NO: 706 - protein, 7 aa, Homo sapiens
DNNKRPS

SEQ ID NO: 707 - protein, 11 aa, Homo sapiens
LTWTGAGRIFV

SEQ ID NO: 708 - protein, 110 aa, Homo sapiens; identical to SEQ ID NO: 428

SEQ ID NO: 709 - DNA, 363 bp, Homo sapiens
gaagtgcagctgcttgaatctggcggaggactggttcaacctggcggctctctgagactgtcttgtgccgccagcggcttcacctttaagcacagcgccattagctgggtccgacaggcccctggaaaaggacttgaatgggtgtccgccatcagctatggcggcggaagccaatattacgccgatagcgtgaagggcagattcaccatcagccgggacaacagcaagaacaccctgtacctgcagatgaacagcctgagagccgaggacaccgccgtgtactactgtgccagagacagcagagatgcctacggcgtggcatttgatctgtggggccagggcacaatggtcacagtctcgagc

SEQ ID NO: 710 - DNA, 330 bp, Homo sapiens; identical to SEQ ID NO: 430

SEQ ID NO: 711 - protein, 5 aa, Homo sapiens
FTTLS

SEQ ID NO: 712 - protein, 17 aa, Homo sapiens
AISSAGGSSYYSDSVKG

SEQ ID NO: 713 - protein, 12 aa, Homo sapiens
DSRDAYGVAFDL

SEQ ID NO: 714 - protein, 121 aa, Homo sapiens
EVQLLESGGGLVQPGGSLRLSCAASGFTFSFTTLSWVRQAPGKGLEWVSAISSAGGSSYYSDSVKGRFTISRDNSKNTLYLQMNSLRAEDTAVYYCARDSRDAYGVAFDLWGQGTMVTVSS

SEQ ID NO: 715 - protein, 13 aa, Homo sapiens
SGSSSNIGNNYVS

SEQ ID NO: 716 - protein, 7 aa, Homo sapiens
DNNKRPS

SEQ ID NO: 717 - protein, 11 aa, Homo sapiens
LTWTGAGRIFV

SEQ ID NO: 718 - protein, 110 aa, Homo sapiens; identical to SEQ ID NO: 428

SEQ ID NO: 719 - DNA, 363 bp, Homo sapiens
gaagtgcagctgcttgaatctggcggaggactggttcaacctggcggctctctgagactgtcttgtgccgccagcggcttcacctttagcttcacaacactgagctgggtccgacaggcccctggaaaaggacttgaatgggtgtccgccatcagctctgccggcggaagcagctattacagcgatagcgtgaagggcagattcaccatcagccgggacaacagcaagaacaccctgtacctgcagatgaacagcctgagagccgaggacaccgccgtgtactactgtgccagagacagcagagatgcctacggcgtggcatttgatctgtggggccagggcacaatggtcacagtctcgagc

SEQ ID NO: 720 - DNA, 330 bp, Homo sapiens; identical to SEQ ID NO: 430

SEQ ID NO: 721 - protein, 5 aa, Homo sapiens
TSSLS

SEQ ID NO: 722 - protein, 17 aa, Homo sapiens
AASYSGSSQYYADSVKG

SEQ ID NO: 723 - protein, 12 aa, Homo sapiens
DSRDAYGVAFDL

SEQ ID NO: 724 - protein, 121 aa, Homo sapiens
EVQLLESGGGLVQPGGSLRLSCAASGFTFTTSSLSWVRQAPGKGLEWVSAASYSGSSQYYADSVKGRFTISRDNSKNTLYLQMNSLRAEDTAVYYCARDSRDAYGVAFDLWGQGTMVTVSS

SEQ ID NO: 725 - protein, 13 aa, Homo sapiens
SGSSSNIGNNYVS

SEQ ID NO: 726 - protein, 7 aa, Homo sapiens
DNNKRPS

SEQ ID NO: 727 - protein, 11 aa, Homo sapiens
LTWTGAGRIFV

SEQ ID NO: 728 - protein, 110 aa, Homo sapiens; identical to SEQ ID NO: 428

SEQ ID NO: 729 - DNA, 363 bp, Homo sapiens
gaagtgcagctgcttgaatctggcggaggactggttcaacctggcggctctctgagactgtcttgtgccgccagcggcttcacctttacaacaagcagccttagctgggtccgacaggcccctggaaaaggacttgaatgggtgtccgccgccagctactctggctctagccaatattacgccgatagcgtgaagggcagattcaccatcagccgggacaacagcaagaacaccctgtacctgcagatgaacagcctgagagccgaggacaccgccgtgtactactgtgccagagacagcagagatgcctacggcgtggcatttgatctgtggggccagggcacaatggtcacagtctcgagc

SEQ ID NO: 730 - DNA, 330 bp, Homo sapiens; identical to SEQ ID NO: 430

SEQ ID NO: 731 - protein, 5 aa, Homo sapiens
TSALS

SEQ ID NO: 732 - protein, 17 aa, Homo sapiens
AISYAGSGQYYADSVKG

SEQ ID NO: 733 - protein, 12 aa, Homo sapiens
DSRDAYGVAFDL

SEQ ID NO: 734 - protein, 121 aa, Homo sapiens
EVQLLESGGGLVQPGGSLRLSCAASGFTFSTSALSWVRQAPGKGLEWVSAISYAGSGQYYADSVKGRFTISRDNSKNTLYLQMNSLRAEDTAVYYCARDSRDAYGVAFDLWGQGTMVTVSS

SEQ ID NO: 735 - protein, 13 aa, Homo sapiens
SGSSSNIGNNYVS

SEQ ID NO: 736 - protein, 7 aa, Homo sapiens
DNNKRPS

SEQ ID NO: 737 - protein, 11 aa, Homo sapiens
LTWTGAGRIFV

SEQ ID NO: 738 - protein, 110 aa, Homo sapiens; identical to SEQ ID NO: 428

SEQ ID NO: 739 - DNA, 363 bp, Homo sapiens
gaagtgcagctgcttgaatctggcggaggactggttcaacctggcggctctctgagactgtcttgtgccgccagcggcttcacctttagcacaagcgcccttagctgggtccgacaggcccctggaaaaggacttgaatgggtgtccgccatcagctatgccggctctggccaatattacgccgatagcgtgaagggcagattcaccatcagccgggacaacagcaagaacaccctgtacctgcagatgaacagcctgagagccgaggacaccgccgtgtactactgtgccagagacagcagagatgcctacggcgtggcatttgatctgtggggccagggcacaatggtcacagtctcgagc

SEQ ID NO: 740 - DNA, 330 bp, Homo sapiens; identical to SEQ ID NO: 430

SEQ ID NO: 741 - protein, 5 aa, Homo sapiens
TSALS

SEQ ID NO: 742 - protein, 17 aa, Homo sapiens
AISYGGGSHYYADSVKG

SEQ ID NO: 743 - protein, 12 aa, Homo sapiens
DSRDAYGVAFDL

SEQ ID NO: 744 - protein, 121 aa, Homo sapiens
EVQLLESGGGLVQPGGSLRLSCAASGFTFATSALSWVRQAPGKGLEWVSAISYGGGSHYYADSVKGRFTISRDNSKNTLYLQMNSLRAEDTAVYYCARDSRDAYGVAFDLWGQGTMVTVSS

SEQ ID NO: 745 - protein, 13 aa, Homo sapiens
SGSSSNIGNNYVS

SEQ ID NO: 746 - protein, 7 aa, Homo sapiens
DNNKRPS

SEQ ID NO: 747 - protein, 11 aa, Homo sapiens
LTWTGAGRIFV

SEQ ID NO: 748 - protein, 110 aa, Homo sapiens; identical to SEQ ID NO: 428

SEQ ID NO: 749 - DNA, 363 bp, Homo sapiens
gaagtgcagctgcttgaatctggcggaggactggttcaacctggcggctctctgagactgtcttgtgccgccagcggcttcacctttgctacaagcgcccttagctgggtccgacaggcccctggaaaaggacttgaatgggtgtccgccatcagctatggcggcggaagccattattacgccgatagcgtgaagggcagattcaccatcagccgggacaacagcaagaacaccctgtacctgcagatgaacagcctgagagccgaggacaccgccgtgtactactgtgccagagacagcagagatgcctacggcgtggcatttgatctgtggggccagggcacaatggtcacagtctcgagc

SEQ ID NO: 750 - DNA, 330 bp, Homo sapiens; identical to SEQ ID NO: 430

SEQ ID NO: 751 - protein, 5 aa, Homo sapiens
FYSLS

SEQ ID NO: 752 - protein, 17 aa, Homo sapiens
AISGGGGGSYYADSVKG

SEQ ID NO: 753 - protein, 12 aa, Homo sapiens
DSRDAYGVAFDL

SEQ ID NO: 754 - protein, 121 aa, Homo sapiens
EVQLLESGGGLVQPGGSLRLSCAASGFTFSFYSLSWVRQAPGKGLEWVSAISGGGGGSYYADSVKGRFTISRDNSKNTLYLQMNSLRAEDTAVYYCARDSRDAYGVAFDLWGQGTMVTVSS

SEQ ID NO: 755 - protein, 13 aa, Homo sapiens
SGSSSNIGNNYVS

SEQ ID NO: 756 - protein, 7 aa, Homo sapiens
DNNKRPS

SEQ ID NO: 757 - protein, 11 aa, Homo sapiens
LTWTGAGRIFV

SEQ ID NO: 758 - protein, 110 aa, Homo sapiens; identical to SEQ ID NO: 428

SEQ ID NO: 759 - DNA, 363 bp, Homo sapiens
gaagtgcagctgcttgaatctggcggaggactggttcaacctggcggctctctgagactgtcttgtgccgccagcggcttcacctttagcttctattctctgagctgggtccgacaggcccctggaaaaggacttgaatgggtgtccgccatcagcggcggaggcggaggaagctattacgccgatagcgtgaagggcagattcaccatcagccgggacaacagcaagaacaccctgtacctgcagatgaacagcctgagagccgaggacaccgccgtgtactactgtgccagagacagcagagatgcctacggcgtggcatttgatctgtggggccagggcacaatggtcacagtctcgagc

SEQ ID NO: 760 - DNA, 330 bp, Homo sapiens; identical to SEQ ID NO: 430

SEQ ID NO: 761 - protein, 5 aa, Homo sapiens
QTAMS

SEQ ID NO: 762 - protein, 17 aa, Homo sapiens
AASYSGYSTYYADSVKG

SEQ ID NO: 763 - protein, 12 aa, Homo sapiens
DSRDAYGVAFDL

SEQ ID NO: 764 - protein, 121 aa, Homo sapiens
EVQLLESGGGLVQPGGSLRLSCAASGFTFSQTAMSWVRQAPGKGLEWVSAASYSGYSTYYADSVKGRFTISRDNSKNTLYLQMNSLRAEDTAVYYCARDSRDAYGVAFDLWGQGTMVTVSS

SEQ ID NO: 765 - protein, 13 aa, Homo sapiens
SGSSSNIGNNYVS

SEQ ID NO: 766 - protein, 7 aa, Homo sapiens
DNNKRPS

SEQ ID NO: 767 - protein, 11 aa, Homo sapiens
LTWTGAGRIFV

SEQ ID NO: 768 - protein, 110 aa, Homo sapiens; identical to SEQ ID NO: 428

SEQ ID NO: 769 - DNA, 363 bp, Homo sapiens
gaagtgcagctgcttgaatctggcggaggactggttcaacctggcggctctctgagactgtcttgtgccgccagcggcttcacctttagccaaacagccatgagctgggtccgacaggcccctggaaaaggacttgaatgggtgtccgccgccagctactctggctacagcacatattacgccgatagcgtgaagggcagattcaccatcagccgggacaacagcaagaacaccctgtacctgcagatgaacagcctgagagccgaggacaccgccgtgtactactgtgccagagacagcagagatgcctacggcgtggcatttgatctgtggggccagggcacaatggtcacagtctcgagc

SEQ ID NO: 770 - DNA, 330 bp, Homo sapiens; identical to SEQ ID NO: 430

SEQ ID NO: 771 - protein, 5 aa, Homo sapiens
SSAVS

SEQ ID NO: 772 - protein, 17 aa, Homo sapiens
AISYYGGAQYYADSVKG

SEQ ID NO: 773 - protein, 12 aa, Homo sapiens
DSRDAYGVAFDL

SEQ ID NO: 774 - protein, 121 aa, Homo sapiens
EVQLLESGGGLVQPGGSLRLSCAASGFTFSSSAVSWVRQAPGKGLEWVSAISYYGGAQYYADSVKGRFTISRDNSKNTLYLQMNSLRAEDTAVYYCARDSRDAYGVAFDLWGQGTMVTVSS

SEQ ID NO: 775 - protein, 13 aa, Homo sapiens
SGSSSNIGNNYVS

SEQ ID NO: 776 - protein, 7 aa, Homo sapiens
DNNKRPS

SEQ ID NO: 777 - protein, 11 aa, Homo sapiens
LTWTGAGRIFV

SEQ ID NO: 778 - protein, 110 aa, Homo sapiens; identical to SEQ ID NO: 428

SEQ ID NO: 779 - DNA, 363 bp, Homo sapiens
gaagtgcagctgcttgaatctggcggaggactggttcaacctggcggctctctgagactgtcttgtgccgccagcggcttcacctttagcagctctgccgttagctgggtccgacaggcccctggaaaaggacttgaatgggtgtccgccatcagctactacggcggagcacagtattacgccgatagcgtgaagggcagattcaccatcagccgggacaacagcaagaacaccctgtacctgcagatgaacagcctgagagccgaggacaccgccgtgtactactgtgccagagacagcagagatgcctacggcgtggcatttgatctgtggggccagggcacaatggtcacagtctcgagc

SEQ ID NO: 780 - DNA, 330 bp, Homo sapiens; identical to SEQ ID NO: 430

SEQ ID NO: 781 - protein, 5 aa, Homo sapiens
SYAMS

SEQ ID NO: 782 - protein, 17 aa, Homo sapiens
AISGSGGSTYYADSVKG

SEQ ID NO: 783 - protein, 12 aa, Homo sapiens
DSRDAYGVAFDL

SEQ ID NO: 784 - protein, 121 aa, Homo sapiens; identical to SEQ ID NO: 414

SEQ ID NO: 785 - protein, 13 aa, Homo sapiens
SGSSSNIGNNYVS

SEQ ID NO: 786 - protein, 7 aa, Homo sapiens
DNNKRPS

SEQ ID NO: 787 - protein, 11 aa, Homo sapiens
LSYKLSPGAYV

SEQ ID NO: 788 - protein, 110 aa, Homo sapiens
ASVLTQPPSVSAAPGQKVTISCSGSSSNIGNNYVSWYQQLPGTAPKLLIYDNNKRPSGIPDRFSGSKSGTSATLGITGLQAEDEADYYCLSYKLSPGAYVFGGGTKLTVL

SEQ ID NO: 789 - DNA, 363 bp, Homo sapiens; identical to SEQ ID NO: 419

SEQ ID NO: 790 - DNA, 330 bp, Homo sapiens
gcctctgtgctgacacagcctccatccgtttctgctgcccctggccagaaagtgaccatcagctgtagcggcagcagcagcaacatcggcaacaactacgtgtcctggtatcagcagctgcccggcacagctcccaaactgctgatctacgacaacaacaagcggcccagcggcatccccgatagattttctggcagcaagagcggcaccagcgccacactgggaattacaggactgcaggccgaggacgaggccgactactactgtctgagctacaagctgagccctggcgcctatgtgtttggcggaggtaccaagctgacagtgctg

SEQ ID NO: 791 - protein, 5 aa, Homo sapiens
SYAMS

SEQ ID NO: 792 - protein, 17 aa, Homo sapiens
AISGSGGSTYYADSVKG

SEQ ID NO: 793 - protein, 12 aa, Homo sapiens
DSRDAYGVAFDL

SEQ ID NO: 794 - protein, 121 aa, Homo sapiens; identical to SEQ ID NO: 414

SEQ ID NO: 795 - protein, 13 aa, Homo sapiens
SGSSSNIGNNYVS

SEQ ID NO: 796 - protein, 7 aa, Homo sapiens
DNNKRPS

SEQ ID NO: 797 - protein, 9 aa, Homo sapiens
QSYGHRSFV

SEQ ID NO: 798 - protein, 108 aa, Homo sapiens
ASVLTQPPSVSAAPGQKVTISCSGSSSNIGNNYVSWYQQLPGTAPKLLIYDNNKRPSGIPDRFSGSKSGTSATLGITGLQAEDEADYYCQSYGHRSFVFGGGTKLTVL

SEQ ID NO: 799 - DNA, 363 bp, Homo sapiens; identical to SEQ ID NO: 419

SEQ ID NO: 800 - DNA, 324 bp, Homo sapiens
gcctctgtgctgacacagcctccatctgtttctgctgcccctggccagaaagtgaccatcagctgtagcggcagcagcagcaacatcggcaacaactacgtgtcctggtatcagcagctgcccggaacagcccctaaactgctgatctacgacaacaacaagcggcccagcggcatccctgatagattttctggcagcaagagcggcacaagcgccacactgggaattacaggactgcaggccgaggacgaggccgattactactgtcagtcttacggccaccggtccttcgtgttcggcggcggtaccaagctgacagtgctg

SEQ ID NO: 801 - protein, 5 aa, Homo sapiens
SYAMS

SEQ ID NO: 802 - protein, 17 aa, Homo sapiens
AISGSGGSTYYADSVKG

SEQ ID NO: 803 - protein, 12 aa, Homo sapiens
DSRDAYGVAFDL

SEQ ID NO: 804 - protein, 121 aa, Homo sapiens; identical to SEQ ID NO: 414

SEQ ID NO: 805 - protein, 13 aa, Homo sapiens
SGSSSNIGNNYVS

SEQ ID NO: 806 - protein, 7 aa, Homo sapiens
DNNKRPS

SEQ ID NO: 807 - protein, 9 aa, Homo sapiens
QSYESRLFV

SEQ ID NO: 808 - protein, 108 aa, Homo sapiens
ASVLTQPPSVSAAPGQKVTISCSGSSSNIGNNYVSWYQQLPGTAPKLLIYDNNKRPSGIPDRFSGSKSGTSATLGITGLQAEDEADYYCQSYESRLFVFGGGTKLTVL

SEQ ID NO: 809 - DNA, 363 bp, Homo sapiens; identical to SEQ ID NO: 419

SEQ ID NO: 810 - DNA, 324 bp, Homo sapiens
gcctctgtgctgacacagcctccatctgtttctgctgcccctggccagaaagtgaccatcagctgtagcggcagcagcagcaacatcggcaacaactacgtgtcctggtatcagcagctgcccggaacagcccctaaactgctgatctacgacaacaacaagcggcccagcggcatccctgatagattttctggcagcaagagcggcacaagcgccacactgggaattacaggactgcaggccgaggacgaggccgattactactgtcagagctacgagagccggctgttcgtgttcggcggcggtaccaagctgacagtgctg

SEQ ID NO: 811 - protein, 5 aa, Homo sapiens
FSALS

SEQ ID NO: 812 - protein, 17 aa, Homo sapiens
AISYGGGSKYYADSVKG

SEQ ID NO: 813 - protein, 12 aa, Homo sapiens
DSRDAYGVAFDL

SEQ ID NO: 814 - protein, 121 aa, Homo sapiens; identical to SEQ ID NO: 684

SEQ ID NO: 815 - protein, 13 aa, Homo sapiens
SGSSSNIGNNYVS

SEQ ID NO: 816 - protein, 7 aa, Homo sapiens
DNNKRPS

SEQ ID NO: 817 - protein, 11 aa, Homo sapiens
LSYKLSPGAYV

SEQ ID NO: 818 - protein, 110 aa, Homo sapiens; identical to SEQ ID NO: 788

SEQ ID NO: 819 - DNA, 363 bp, Homo sapiens; identical to SEQ ID NO: 689

SEQ ID NO: 820 - DNA, 330 bp, Homo sapiens; identical to SEQ ID NO: 790

SEQ ID NO: 821 - protein, 5 aa, Homo sapiens
FSALS

SEQ ID NO: 822 - protein, 17 aa, Homo sapiens
AISYGGGSKYYADSVKG

SEQ ID NO: 823 - protein, 12 aa, Homo sapiens
DSRDAYGVAFDL

SEQ ID NO: 824 - protein, 121 aa, Homo sapiens; identical to SEQ ID NO: 684

SEQ ID NO: 825 - protein, 13 aa, Homo sapiens
SGSSSNIGNNYVS

SEQ ID NO: 826 - protein, 7 aa, Homo sapiens
DNNKRPS

SEQ ID NO: 827 - protein, 9 aa, Homo sapiens
QSYESRLFV

SEQ ID NO: 828 - protein, 108 aa, Homo sapiens; identical to SEQ ID NO: 808

SEQ ID NO: 829 - DNA, 363 bp, Homo sapiens; identical to SEQ ID NO: 689

SEQ ID NO: 830 - DNA, 324 bp, Homo sapiens; identical to SEQ ID NO: 810

SEQ ID NO: 831 - protein, 5 aa, Homo sapiens
HSAIS

SEQ ID NO: 832 - protein, 17 aa, Homo sapiens
AISYGGGSQYYADSVKG

SEQ ID NO: 833 - protein, 12 aa, Homo sapiens
DSRDAYGVAFDL

SEQ ID NO: 834 - protein, 121 aa, Homo sapiens; identical to SEQ ID NO: 704

SEQ ID NO: 835 - protein, 13 aa, Homo sapiens
SGSSSNIGNNYVS

SEQ ID NO: 836 - protein, 7 aa, Homo sapiens
DNNKRPS

SEQ ID NO: 837 - protein, 9 aa, Homo sapiens
QSYGHRSFV

SEQ ID NO: 838 - protein, 108 aa, Homo sapiens; identical to SEQ ID NO: 798

SEQ ID NO: 839 - DNA, 363 bp, Homo sapiens; identical to SEQ ID NO: 709

SEQ ID NO: 840 - DNA, 324 bp, Homo sapiens; identical to SEQ ID NO: 800

SEQ ID NO: 841 - protein, 5 aa, Homo sapiens
HAWIS

SEQ ID NO: 842 - protein, 19 aa, Homo sapiens
QIKGGPGSGGTSYAEPVKG

SEQ ID NO: 843 - protein, 9 aa, Homo sapiens
LGIYSGFDY

SEQ ID NO: 844 - protein, 120 aa, Homo sapiens
EVQLVESGGGLVKPGGSLRLSCAASGFTFSHAWISWVRQAPGKGLEWVGQIKGGPGSGGTSYAEPVKGRFTISRDDSKNTLYLQMNSLKTEDTAVYYCARLGIYSGFDYWGQGTLVTVSS

SEQ ID NO: 845 - protein, 11 aa, Homo sapiens
SGDKLGDKYAS

SEQ ID NO: 846 - protein, 7 aa, Homo sapiens
QDSKRPS

SEQ ID NO: 847 - protein, 11 aa, Homo sapiens
HSYTGKPSQVV

SEQ ID NO: 848 - protein, 108 aa, Homo sapiens; identical to SEQ ID NO: 438

SEQ ID NO: 849 - DNA, 360 bp, Homo sapiens
gaagtgcagctggttgaatctggcggcggactggttaagcctggcggatctctgagactgagctgtgccgccagcggcttcacattttctcacgcctggatcagctgggtccgacaggctcctggaaaaggcctggaatgggtcggacagatcaaaggcggacctggctctggcggaacaagctatgccgagcctgtgaagggcagattcaccatcagccgggacgacagcaagaacaccctgtacctgcagatgaacagcctgaaaaccgaggacaccgccgtgtactactgtgccagactgggcatctactccggcttcgattattggggccagggcaccctggttacagtctcgagc

SEQ ID NO: 850 - DNA, 324 bp, Homo sapiens; identical to SEQ ID NO: 440

SEQ ID NO: 851 - protein, 5 aa, Homo sapiens
YAWIS

SEQ ID NO: 852 - protein, 19 aa, Homo sapiens
QIKSGSDASQTSYAAPVKG

SEQ ID NO: 853 - protein, 9 aa, Homo sapiens
LGIYSGFDY

SEQ ID NO: 854 - protein, 120 aa, Homo sapiens
EVQLVESGGGLVKPGGSLRLSCAASGFTFTYAWISWVRQAPGKGLEWVGQIKSGSDASQTSYAAPVKGRFTISRDDSKNTLYLQMNSLKTEDTAVYYCARLGIYSGFDYWGQGTLVTVSS

SEQ ID NO: 855 - protein, 11 aa, Homo sapiens
SGDKLGDKYAS

SEQ ID NO: 856 - protein, 7 aa, Homo sapiens
QDSKRPS

SEQ ID NO: 857 - protein, 11 aa, Homo sapiens
HSYTGKPSQVV

SEQ ID NO: 858 - protein, 108 aa, Homo sapiens; identical to SEQ ID NO: 438

SEQ ID NO: 859 - DNA, 360 bp, Homo sapiens
gaagtgcagctggttgaatctggcggcggacttgtgaaacctggcggctctctgagactgtcttgtgccgccagcggcttcaccttcacatacgcctggattagctgggttcgacaggcccctggaaaaggcctggaatgggtcggacagatcaagagcggaagcgacgccagccagacatcttacgctgctccagtgaagggcagattcaccatcagccgggacgacagcaagaacaccctgtacctgcagatgaacagcctgaaaaccgaggacaccgccgtgtactactgcgccagactgggcatctacagcggcttcgactactggggccagggcaccctggtgaccgtctcgagc

SEQ ID NO: 860 - DNA, 324 bp, Homo sapiens; identical to SEQ ID NO: 440

SEQ ID NO: 861 - protein, 5 aa, Homo sapiens
SLWMS

SEQ ID NO: 862 - protein, 19 aa, Homo sapiens
QIKSSTSGSGTSYGAPVKG

SEQ ID NO: 863 - protein, 9 aa, Homo sapiens
LGIYSGFDY

SEQ ID NO: 864 - protein, 120 aa, Homo sapiens
EVQLVESGGGLVKPGGSLRLSCAASGFTFSSLWMSWVRQAPGKGLEWVGQIKSSTSGSGTSYGAPVKGRFTISRDDSKNTLYLQMNSLKTEDTAVYYCARLGIYSGFDYWGQGTLVTVSS

SEQ ID NO: 865 - protein, 11 aa, Homo sapiens
SGDKLGDKYAS

SEQ ID NO: 866 - protein, 7 aa, Homo sapiens
QDSKRPS

SEQ ID NO: 867 - protein, 11 aa, Homo sapiens
HSYTGKPSQVV

SEQ ID NO: 868 - protein, 108 aa, Homo sapiens; identical to SEQ ID NO: 438

SEQ ID NO: 869 - DNA, 360 bp, Homo sapiens
gaagtgcagctggttgaatctggcggcggacttgtgaaacctggcggctctctgagactgtcttgtgccgccagcggcttcaccttcagcagtctttggatgagctgggttcgacaggcccctggaaaaggcctggaatgggtcggacaaatcaagagcagtaccagcggctctggcacctcttacggtgctccagtgaagggcagattcaccatcagccgggacgacagcaagaacaccctgtacctgcagatgaacagcctgaaaaccgaggacaccgccgtgtactactgcgccagactgggcatctacagcggcttcgactactggggccagggcaccctggtgaccgtctcgagc

SEQ ID NO: 870 - DNA, 324 bp, Homo sapiens; identical to SEQ ID NO: 440

SEQ ID NO: 871 - protein, 5 aa, Homo sapiens
SAWIS

SEQ ID NO: 872 - protein, 19 aa, Homo sapiens
QIKSKSEASSTTYAAPVKG

SEQ ID NO: 873 - protein, 9 aa, Homo sapiens
LGIYSGFDY

SEQ ID NO: 874 - protein, 120 aa, Homo sapiens
EVQLVESGGGLVKPGGSLRLSCAASGFTFESAWISWVRQAPGKGLEWVGQIKSKSEASSTTYAAPVKGRFTISRDDSKNTLYLQMNSLKTEDTAVYYCARLGIYSGFDYWGQGTLVTVSS

SEQ ID NO: 875 - protein, 11 aa, Homo sapiens
SGDKLGDKYAS

SEQ ID NO: 876 - protein, 7 aa, Homo sapiens
QDSKRPS

SEQ ID NO: 877 - protein, 11 aa, Homo sapiens
HSYTGKPSQVV

SEQ ID NO: 878 - protein, 108 aa, Homo sapiens; identical to SEQ ID NO: 438

SEQ ID NO: 879 - DNA, 360 bp, Homo sapiens
gaagtgcagctggttgaatctggcggcggacttgtgaaacctggcggctctctgagactgtcttgtgccgccagcggcttcaccttcgaaagcgcctggattagctgggttcgacaggcccctggaaaaggcctggaatgggtcggacagatcaagagcaagagcgaggccagcagcaccacatacgctgctccagtgaagggcagattcaccatcagccgggacgacagcaagaacaccctgtacctgcagatgaacagcctgaaaaccgaggacaccgccgtgtactactgcgccagactgggcatctacagcggcttcgactactggggccagggcaccctggtgaccgtctcgagc

SEQ ID NO: 880 - DNA, 324 bp, Homo sapiens; identical to SEQ ID NO: 440

SEQ ID NO: 881 - protein, 5 aa, Homo sapiens
HAWMS

SEQ ID NO: 882 - protein, 19 aa, Homo sapiens
QIKSKSDASKTTYAAPVKG

SEQ ID NO: 883 - protein, 9 aa, Homo sapiens
LGIYSGFDY

SEQ ID NO: 884 - protein, 120 aa, Homo sapiens
EVQLVESGGGLVKPGGSLRLSCAASGFTFSHAWMSWVRQAPGKGLEWVGQIKSKSDASKTTYAAPVKGRFTISRDDSKNTLYLQMNSLKTEDTAVYYCARLGIYSGFDYWGQGTLVTVSS

SEQ ID NO: 885 - protein, 11 aa, Homo sapiens
SGDKLGDKYAS

SEQ ID NO: 886 - protein, 7 aa, Homo sapiens
QDSKRPS

SEQ ID NO: 887 - protein, 11 aa, Homo sapiens
HSYTGKPSQVV

SEQ ID NO: 888 - protein, 108 aa, Homo sapiens; identical to SEQ ID NO: 438

SEQ ID NO: 889 - DNA, 360 bp, Homo sapiens
gaagtgcagctggttgaatctggcggcggacttgtgaaacctggcggctctctgagactgtcttgtgccgccagcggcttcaccttcagccatgcctggatgagctgggttcgacaggcccctggaaaaggcctggaatgggtcggacagatcaagagcaagagcgacgccagcaagaccacatacgctgctccagtgaagggcagattcaccatcagccgggacgacagcaagaacaccctgtacctgcagatgaacagcctgaaaaccgaggacaccgccgtgtactactgcgccagactgggcatctacagcggcttcgactactggggccagggcaccctggtgaccgtctcgagc

SEQ ID NO: 890 - DNA, 324 bp, Homo sapiens; identical to SEQ ID NO: 440

SEQ ID NO: 891 - protein, 5 aa, Homo sapiens
YTWIS

SEQ ID NO: 892 - protein, 19 aa, Homo sapiens
QIKSTTSASSIDYASPVKG

SEQ ID NO: 893 - protein, 9 aa, Homo sapiens
LGIYSGFDY

SEQ ID NO: 894 - protein, 120 aa, Homo sapiens
EVQLVESGGGLVKPGGSLRLSCAASGFTFSYTWISWVRQAPGKGLEWVGQIKSTTSASSIDYASPVKGRFTISRDDSKNTLYLQMNSLKTEDTAVYYCARLGIYSGFDYWGQGTLVTVSS

SEQ ID NO: 895 - protein, 11 aa, Homo sapiens
SGDKLGDKYAS

SEQ ID NO: 896 - protein, 7 aa, Homo sapiens
QDSKRPS

SEQ ID NO: 897 - protein, 11 aa, Homo sapiens
HSYTGKPSQVV

SEQ ID NO: 898 - protein, 108 aa, Homo sapiens; identical to SEQ ID NO: 438

SEQ ID NO: 899 - DNA, 360 bp, Homo sapiens
gaagtgcagctggttgaatctggcggcggacttgtgaaacctggcggctctctgagactgtcttgtgccgccagcggcttcaccttcagctacacatggatcagctgggttcgacaggcccctggaaaaggcctggaatgggtcggacaaatcaagagcaccaccagcgccagcagcatcgattacgcttctccagtgaagggcagattcaccatcagccgggacgacagcaagaacaccctgtacctgcagatgaacagcctgaaaaccgaggacaccgccgtgtactactgcgccagactgggcatctacagcggcttcgactactggggccagggcaccctggtgaccgtctcgagc

SEQ ID NO: 900 - DNA, 324 bp, Homo sapiens; identical to SEQ ID NO: 440

SEQ ID NO: 901 - protein, 5 aa, Homo sapiens
YAYMY

SEQ ID NO: 902 - protein, 19 aa, Homo sapiens
HIKSSTDGSGKEYSAPVKG

SEQ ID NO: 903 - protein, 9 aa, Homo sapiens
LGIYSGFDY

SEQ ID NO: 904 - protein, 120 aa, Homo sapiens
EVQLVESGGGLVKPGGSLRLSCAASGFTFTYAYMYWVRQAPGKGLEWVGHIKSSTDGSGKEYSAPVKGRFTISRDDSKNTLYLQMNSLKTEDTAVYYCARLGIYSGFDYWGQGTLVTVSS

SEQ ID NO: 905 - protein, 11 aa, Homo sapiens
SGDKLGDKYAS

SEQ ID NO: 906 - protein, 7 aa, Homo sapiens
QDSKRPS

SEQ ID NO: 907 - protein, 11 aa, Homo sapiens
HSYTGKPSQVV

SEQ ID NO: 908 - protein, 108 aa, Homo sapiens; identical to SEQ ID NO: 438

SEQ ID NO: 909 - DNA, 360 bp, Homo sapiens
gaagtgcagctggttgaatctggcggcggacttgtgaaacctggcggctctctgagactgtcttgtgccgccagcggcttcaccttcacctacgcctacatgtactgggttcgacaggcccctggaaaaggcctggaatgggtcggacatatcaagagcagtaccgacggcagcggcaaagaatactctgctccagtgaagggcagattcaccatcagccgggacgacagcaagaacaccctgtacctgcagatgaacagcctgaaaaccgaggacaccgccgtgtactactgcgccagactgggcatctacagcggcttcgactactggggccagggcaccctggtgaccgtctcgagc

SEQ ID NO: 910 - DNA, 324 bp, Homo sapiens; identical to SEQ ID NO: 440

SEQ ID NO: 911 - protein, 5 aa, Homo sapiens
YAWIS

SEQ ID NO: 912 - protein, 19 aa, Homo sapiens
QIKSSSDASSTTYAAPVKG

SEQ ID NO: 913 - protein, 9 aa, Homo sapiens
LGIYSGFDY

SEQ ID NO: 914 - protein, 120 aa, Homo sapiens
EVQLVESGGGLVKPGGSLRLSCAASGFTFSYAWISWVRQAPGKGLEWVGQIKSSSDASSTTYAAPVKGRFTISRDDSKNTLYLQMNSLKTEDTAVYYCARLGIYSGFDYWGQGTLVTVSS

SEQ ID NO: 915 - protein, 11 aa, Homo sapiens
SGDKLGDKYAS

SEQ ID NO: 916 - protein, 7 aa, Homo sapiens
QDSKRPS

SEQ ID NO: 917 - protein, 11 aa, Homo sapiens
HSYTGKPSQVV

SEQ ID NO: 918 - protein, 108 aa, Homo sapiens; identical to SEQ ID NO: 438

SEQ ID NO: 919 - DNA, 360 bp, Homo sapiens
gaagtgcagctggttgaatctggcggcggacttgtgaaacctggcggctctctgagactgtcttgtgccgccagcggcttcaccttcagctatgcctggattagctgggttcgacaggcccctggaaaaggcctggaatgggtcggacagatcaagagcagcagcgacgccagctctaccacatacgctgctccagtgaagggcagattcaccatcagccgggacgacagcaagaacaccctgtacctgcagatgaacagcctgaaaaccgaggacaccgccgtgtactactgcgccagactgggcatctacagcggcttcgactactggggccagggcaccctggtgaccgtctcgagc

SEQ ID NO: 920 - DNA, 324 bp, Homo sapiens; identical to SEQ ID NO: 440

SEQ ID NO: 921 - protein, 5 aa, Homo sapiens
HAWIT

SEQ ID NO: 922 - protein, 19 aa, Homo sapiens
QIKSSSDASETSYAAPVKG

SEQ ID NO: 923 - protein, 9 aa, Homo sapiens
LGIYSGFDY

SEQ ID NO: 924 - protein, 120 aa, Homo sapiens
EVQLVESGGGLVKPGGSLRLSCAASGFTFSHAWITWVRQAPGKGLEWVGQIKSSSDASETSYAAPVKGRFTISRDDSKNTLYLQMNSLKTEDTAVYYCARLGIYSGFDYWGQGTLVTVSS

SEQ ID NO: 925 - protein, 11 aa, Homo sapiens
SGDKLGDKYAS

SEQ ID NO: 926 - protein, 7 aa, Homo sapiens
QDSKRPS

SEQ ID NO: 927 - protein, 11 aa, Homo sapiens
HSYTGKPSQVV

SEQ ID NO: 928 - protein, 108 aa, Homo sapiens; identical to SEQ ID NO: 438

SEQ ID NO: 929 - DNA, 360 bp, Homo sapiens
gaagtgcagctggttgaatctggcggcggacttgtgaaacctggcggctctctgagactgtcttgtgccgccagcggcttcaccttcagccatgcctggattacatgggttcgacaggcccctggaaaaggcctggaatgggtcggacagatcaagagcagcagcgacgccagcgagacatcttacgctgctccagtgaagggcagattcaccatcagccgggacgacagcaagaacaccctgtacctgcagatgaacagcctgaaaaccgaggacaccgccgtgtactactgcgccagactgggcatctacagcggcttcgactactggggccagggcaccctggtgaccgtctcgagc

SEQ ID NO: 930 - DNA, 324 bp, Homo sapiens; identical to SEQ ID NO: 440

SEQ ID NO: 931 - protein, 5 aa, Homo sapiens
SAWVS

SEQ ID NO: 932 - protein, 19 aa, Homo sapiens
QIKGYTSGGTITYAAPVKG

SEQ ID NO: 933 - protein, 9 aa, Homo sapiens
LGIYSGFDY

SEQ ID NO: 934 - protein, 120 aa, Homo sapiens
EVQLVESGGGLVKPGGSLRLSCAASGFTFSSAWVSWVRQAPGKGLEWVGQIKGYTSGGTITYAAPVKGRFTISRDDSKNTLYLQMNSLKTEDTAVYYCARLGIYSGFDYWGQGTLVTVSS

SEQ ID NO: 935 - protein, 11 aa, Homo sapiens
SGDKLGDKYAS

SEQ ID NO: 936 - protein, 7 aa, Homo sapiens
QDSKRPS

SEQ ID NO: 937 - protein, 11 aa, Homo sapiens
HSYTGKPSQVV

SEQ ID NO: 938 - protein, 108 aa, Homo sapiens; identical to SEQ ID NO: 438

SEQ ID NO: 939 - DNA, 360 bp, Homo sapiens
gaagtgcagctggttgaatctggcggcggacttgtgaaacctggcggctctctgagactgtcttgtgccgccagcggcttcaccttcagctctgcctgggttagctgggttcgacaggcccctggaaaaggcctggaatgggtcggacagatcaagggctataccagcggcggcaccatcacatacgctgctccagtgaagggcagattcaccatcagccgggacgacagcaagaacaccctgtacctgcagatgaacagcctgaaaaccgaggacaccgccgtgtactactgcgccagactgggcatctacagcggcttcgactactggggccagggcaccctggtgaccgtctcgagc

SEQ ID NO: 940 - DNA, 324 bp, Homo sapiens; identical to SEQ ID NO: 440

SEQ ID NO: 941 - protein, 5 aa, Homo sapiens
HAWIS

SEQ ID NO: 942 - protein, 19 aa, Homo sapiens
RIKGSTEASQTDYAAPVKG

SEQ ID NO: 943 - protein, 9 aa, Homo sapiens
LGIYSGFDY

SEQ ID NO: 944 - protein, 120 aa, Homo sapiens
EVQLVESGGGLVKPGGSLRLSCAASGFTFQHAWISWVRQAPGKGLEWVGRIKGSTEASQTDYAAPVKGRFTISRDDSKNTLYLQMNSLKTEDTAVYYCARLGIYSGFDYWGQGTLVTVSS

SEQ ID NO: 945 - protein, 11 aa, Homo sapiens
SGDKLGDKYAS

SEQ ID NO: 946 - protein, 7 aa, Homo sapiens
QDSKRPS

SEQ ID NO: 947 - protein, 11 aa, Homo sapiens
HSYTGKPSQVV

SEQ ID NO: 948 - protein, 108 aa, Homo sapiens; identical to SEQ ID NO: 438

SEQ ID NO: 949 - DNA, 360 bp, Homo sapiens
gaagtgcagctggttgaatctggcggcggacttgtgaaacctggcggctctctgagactgtcttgtgccgccagcggcttcaccttccaacatgcctggattagctgggttcgacaggcccctggaaaaggcctggaatgggtcggaagaatcaagggcagcaccgaggccagccagacagattacgctgctccagtgaagggcagattcaccatcagccgggacgacagcaagaacaccctgtacctgcagatgaacagcctgaaaaccgaggacaccgccgtgtactactgcgccagactgggcatctacagcggcttcgactactggggccagggcaccctggtgaccgtctcgagc

SEQ ID NO: 950 - DNA, 324 bp, Homo sapiens; identical to SEQ ID NO: 440

SEQ ID NO: 951 - protein, 5 aa, Homo sapiens
SLWIS

SEQ ID NO: 952 - protein, 19 aa, Homo sapiens
QIKGKTESSSTTYEAPVKG

SEQ ID NO: 953 - protein, 9 aa, Homo sapiens
LGIYSGFDY

SEQ ID NO: 954 - protein, 120 aa, Homo sapiens
EVQLVESGGGLVKPGGSLRLSCAASGFTFSSLWISWVRQAPGKGLEWVGQIKGKTESSSTTYEAPVKGRFTISRDDSKNTLYLQMNSLKTEDTAVYYCARLGIYSGFDYWGQGTLVTVSS

SEQ ID NO: 955 - protein, 11 aa, Homo sapiens
SGDKLGDKYAS

SEQ ID NO: 956 - protein, 7 aa, Homo sapiens
QDSKRPS

SEQ ID NO: 957 - protein, 11 aa, Homo sapiens
HSYTGKPSQVV

SEQ ID NO: 958 - protein, 108 aa, Homo sapiens; identical to SEQ ID NO: 438

SEQ ID NO: 959 - DNA, 360 bp, Homo sapiens
gaagtgcagctggttgaatctggcggcggacttgtgaaacctggcggctctctgagactgtcttgtgccgccagcggcttcaccttcagcagtctgtggattagctgggttcgacaggcccctggaaaaggcctggaatgggtcggacagatcaagggcaagaccgagagcagcagcaccacatacgaagctccagtgaagggcagattcaccatcagccgggacgacagcaagaacaccctgtacctgcagatgaacagcctgaaaaccgaggacaccgccgtgtactactgcgccagactgggcatctacagcggcttcgactactggggccagggcaccctggtgaccgtctcgagc

SEQ ID NO: 960 - DNA, 324 bp, Homo sapiens; identical to SEQ ID NO: 440

SEQ ID NO: 961 - protein, 5 aa, Homo sapiens
NAWMS

SEQ ID NO: 962 - protein, 19 aa, Homo sapiens
RIKSKTDGGTTDYAAPVKG

SEQ ID NO: 963 - protein, 9 aa, Homo sapiens
LGIYSGFDY

SEQ ID NO: 964 - protein, 120 aa, Homo sapiens; identical to SEQ ID NO: 434

SEQ ID NO: 965 - protein, 11 aa, Homo sapiens
SGDKLGDKYAS

SEQ ID NO: 966 - protein, 7 aa, Homo sapiens
QDSKRPS

SEQ ID NO: 967 - protein, 11 aa, Homo sapiens
HTYSHRPEIVV

SEQ ID NO: 968 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCHTYSHRPEIVVFGGGTKLTVL

SEQ ID NO: 969 - DNA, 360 bp, Homo sapiens; identical to SEQ ID NO: 439

SEQ ID NO: 970 - DNA, 324 bp, Homo sapiens
agctacgagctgacacagcctccaagcgtgtccgtgtctcctggacagacagccagcatcacctgtagcggcgataagctgggcgataagtacgccagctggtatcagcagaagcccggccagtctcctgtgctggtcatctaccaggacagcaagaggcctagcggcatccccgagagattcagcggcagcaatagcggcaataccgccacactgaccatcagcggaacacaggccgaggacgaggccgattactactgccacacctacagccaccggcctgagatcgtggtttttggcggaggtaccaagctgacagtgctg

SEQ ID NO: 971 - protein, 5 aa, Homo sapiens
NAWMS

SEQ ID NO: 972 - protein, 19 aa, Homo sapiens
RIKSKTDGGTTDYAAPVKG

SEQ ID NO: 973 - protein, 9 aa, Homo sapiens
LGIYSGFDY

SEQ ID NO: 974 - protein, 120 aa, Homo sapiens; identical to SEQ ID NO: 434

SEQ ID NO: 975 - protein, 11 aa, Homo sapiens
SGDKLGDKYAS

SEQ ID NO: 976 - protein, 7 aa, Homo sapiens
QDSKRPS

SEQ ID NO: 977 - protein, 11 aa, Homo sapiens
HSYLHRPSVTV

SEQ ID NO: 978 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCHSYLHRPSVTVFGGGTKLTVL

SEQ ID NO: 979 - DNA, 360 bp, Homo sapiens; identical to SEQ ID NO: 439

SEQ ID NO: 980 - DNA, 324 bp, Homo sapiens
agctacgagctgacacagcctcctagcgtttccgtgtctcctggccagacagccagcatcacatgttctggcgacaagctgggcgataagtacgccagctggtatcagcagaagcccggacagtctcccgtgctggtcatctaccaggatagcaagaggcctagcggcatccctgagagattcagcggcagcaatagcggcaataccgccacactgacaatcagcggaacacaggccgaggacgaggccgattactactgtcacagctacctgcacagacccagcgtgacagtgttcggcggcggtaccaagctgacagtgctg

SEQ ID NO: 981 - protein, 5 aa, Homo sapiens
NAWMS

SEQ ID NO: 982 - protein, 19 aa, Homo sapiens
RIKSKTDGGTTDYAAPVKG

SEQ ID NO: 983 - protein, 9 aa, Homo sapiens
LGIYSGFDY

SEQ ID NO: 984 - protein, 120 aa, Homo sapiens; identical to SEQ ID NO: 434

SEQ ID NO: 985 - protein, 11 aa, Homo sapiens
SGDKLGDKYAS

SEQ ID NO: 986 - protein, 7 aa, Homo sapiens
QDSKRPS

SEQ ID NO: 987 - protein, 11 aa, Homo sapiens
HTYLHLPSLVV

SEQ ID NO: 988 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCHTYLHLPSLVVFGGGTKLTVL

SEQ ID NO: 989 - DNA, 360 bp, Homo sapiens; identical to SEQ ID NO: 439

SEQ ID NO: 990 - DNA, 324 bp, Homo sapiens
agctacgagctgacacagcctcctagcgtttccgtgtctcctggccagacagccagcatcacatgttctggcgacaagctgggcgataagtacgccagctggtatcagcagaagcccggacagtctcccgtgctggtcatctaccaggatagcaagaggcctagcggcatccctgagagattcagcggcagcaatagcggcaataccgccacactgacaatcagcggaacacaggccgaggacgaggccgattactactgtcacacatacctgcatctgcccagccttgtggtgttcggcggcggtaccaagctgacagtgctg

SEQ ID NO: 991 - protein, 5 aa, Homo sapiens
NAWMS

SEQ ID NO: 992 - protein, 19 aa, Homo sapiens
RIKSKTDGGTTDYAAPVKG

SEQ ID NO: 993 - protein, 9 aa, Homo sapiens
LGIYSGFDY

SEQ ID NO: 994 - protein, 120 aa, Homo sapiens; identical to SEQ ID NO: 434

SEQ ID NO: 995 - protein, 11 aa, Homo sapiens
SGDKLGDKYAS

SEQ ID NO: 996 - protein, 7 aa, Homo sapiens
QDSKRPS

SEQ ID NO: 997 - protein, 11 aa, Homo sapiens
HSYLHRPETVV

SEQ ID NO: 998 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCHSYLHRPETVVFGGGTKLTVL

SEQ ID NO: 999 - DNA, 360 bp, Homo sapiens; identical to SEQ ID NO: 439

SEQ ID NO: 1000 - DNA, 324 bp, Homo sapiens
agctacgagctgacacagcctcctagcgtttccgtgtctcctggccagacagccagcatcacatgttctggcgacaagctgggcgataagtacgccagctggtatcagcagaagcccggacagtctcccgtgctggtcatctaccaggatagcaagaggcctagcggcatccctgagagattcagcggcagcaatagcggcaataccgccacactgacaatcagcggaacacaggccgaggacgaggccgattactactgtcacagctacctgcacagacccgagacagtggtgttcggcggcggtaccaagctgacagtgctg

SEQ ID NO: 1001 - protein, 5 aa, Homo sapiens
NAWMS

SEQ ID NO: 1002 - protein, 19 aa, Homo sapiens
RIKSKTDGGTTDYAAPVKG

SEQ ID NO: 1003 - protein, 9 aa, Homo sapiens
LGIYSGFDY

SEQ ID NO: 1004 - protein, 120 aa, Homo sapiens; identical to SEQ ID NO: 434

SEQ ID NO: 1005 - protein, 11 aa, Homo sapiens
SGDKLGDKYAS

SEQ ID NO: 1006 - protein, 7 aa, Homo sapiens
QDSKRPS

SEQ ID NO: 1007 - protein, 11 aa, Homo sapiens
HSYSHRPEVVV

SEQ ID NO: 1008 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCHSYSHRPEVVVFGGGTKLTVL

SEQ ID NO: 1009 - DNA, 360 bp, Homo sapiens; identical to SEQ ID NO: 439

SEQ ID NO: 1010 - DNA, 324 bp, Homo sapiens
agctacgagctgacacagcctcctagcgtttccgtgtctcctggccagacagccagcatcacatgttctggcgacaagctgggcgataagtacgccagctggtatcagcagaagcccggacagtctcccgtgctggtcatctaccaggatagcaagaggcctagcggcatccctgagagattcagcggcagcaatagcggcaataccgccacactgacaatcagcggaacacaggccgaggacgaggccgattactactgtcacagctactctcacagacccgaggttgtggtgttcggcggcggtaccaagctgacagtgctg

SEQ ID NO: 1011 - protein, 5 aa, Homo sapiens
NAWMS

SEQ ID NO: 1012 - protein, 19 aa, Homo sapiens
RIKSKTDGGTTDYAAPVKG

SEQ ID NO: 1013 - protein, 9 aa, Homo sapiens
LGIYSGFDY

SEQ ID NO: 1014 - protein, 120 aa, Homo sapiens; identical to SEQ ID NO: 434

SEQ ID NO: 1015 - protein, 11 aa, Homo sapiens
SGDKLGDKYAS

SEQ ID NO: 1016 - protein, 7 aa, Homo sapiens
QDSKRPS

SEQ ID NO: 1017 - protein, 11 aa, Homo sapiens
HSYLHLPPTVV

SEQ ID NO: 1018 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCHSYLHLPPTVVFGGGTKLTVL

SEQ ID NO: 1019 - DNA, 360 bp, Homo sapiens; identical to SEQ ID NO: 439

SEQ ID NO: 1020 - DNA, 324 bp, Homo sapiens
agctacgagctgacacagcctcctagcgtttccgtgtctcctggccagacagccagcatcacatgttctggcgacaagctgggcgataagtacgccagctggtatcagcagaagcccggacagtctcccgtgctggtcatctaccaggatagcaagaggcctagcggcatccctgagagattcagcggcagcaatagcggcaataccgccacactgacaatcagcggaacacaggccgaggacgaggccgattactactgtcacagctacctgcatctgccccctacagtggtgttcggcggcggtaccaagctgacagtgctg

SEQ ID NO: 1021 - protein, 5 aa, Homo sapiens
NAWMS

SEQ ID NO: 1022 - protein, 19 aa, Homo sapiens
RIKSKTDGGTTDYAAPVKG

SEQ ID NO: 1023 - protein, 9 aa, Homo sapiens
LGIYSGFDY

SEQ ID NO: 1024 - protein, 120 aa, Homo sapiens; identical to SEQ ID NO: 434

SEQ ID NO: 1025 - protein, 11 aa, Homo sapiens
SGDKLGDKYAS

SEQ ID NO: 1026 - protein, 7 aa, Homo sapiens
QDSKRPS

SEQ ID NO: 1027 - protein, 12 aa, Homo sapiens
HAYHWKPTPIVV

SEQ ID NO: 1028 - protein, 109 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCHAYHWKPTPIVVFGGGTKLTVL

SEQ ID NO: 1029 - DNA, 360 bp, Homo sapiens; identical to SEQ ID NO: 439

SEQ ID NO: 1030 - DNA, 327 bp, Homo sapiens
agctacgagctgacacagcctcctagcgtttccgtgtctcctggccagacagccagcatcacatgttctggcgacaagctgggcgataagtacgccagctggtatcagcagaagcccggacagtctcccgtgctggtcatctaccaggatagcaagaggcctagcggcatccctgagagattcagcggcagcaatagcggcaataccgccacactgacaatcagcggaacacaggccgaggacgaggccgattactactgtcacgcttaccactggaagcccacacctattgtggtgttcggcggcggtaccaagctgacagtgctg

SEQ ID NO: 1031 - protein, 5 aa, Homo sapiens
NAWMS

SEQ ID NO: 1032 - protein, 19 aa, Homo sapiens
RIKSKTDGGTTDYAAPVKG

SEQ ID NO: 1033 - protein, 9 aa, Homo sapiens
LGIYSGFDY

SEQ ID NO: 1034 - protein, 120 aa, Homo sapiens; identical to SEQ ID NO: 434

SEQ ID NO: 1035 - protein, 11 aa, Homo sapiens
SGDKLGDKYAS

SEQ ID NO: 1036 - protein, 7 aa, Homo sapiens
QDSKRPS

SEQ ID NO: 1037 - protein, 11 aa, Homo sapiens
HTYSHLPPTVV

SEQ ID NO: 1038 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCHTYSHLPPTVVFGGGTKLTVL

SEQ ID NO: 1039 - DNA, 360 bp, Homo sapiens; identical to SEQ ID NO: 439

SEQ ID NO: 1040 - DNA, 324 bp, Homo sapiens
agctacgagctgacacagcctcctagcgtttccgtgtctcctggccagacagccagcatcacatgttctggcgacaagctgggcgataagtacgccagctggtatcagcagaagcccggacagtctcccgtgctggtcatctaccaggatagcaagaggcctagcggcatccctgagagattcagcggcagcaatagcggcaataccgccacactgacaatcagcggaacacaggccgaggacgaggccgattactactgtcacacctactctcatctgccccctacagtggtgttcggcggcggtaccaagctgacagtgctg

SEQ ID NO: 1041 - protein, 5 aa, Homo sapiens
NAWMS

SEQ ID NO: 1042 - protein, 19 aa, Homo sapiens
RIKSKTDGGTTDYAAPVKG

SEQ ID NO: 1043 - protein, 9 aa, Homo sapiens
LGIYSGFDY

SEQ ID NO: 1044 - protein, 120 aa, Homo sapiens; identical to SEQ ID NO: 434

SEQ ID NO: 1045 - protein, 11 aa, Homo sapiens
SGDKLGDKYAS

SEQ ID NO: 1046 - protein, 7 aa, Homo sapiens
QDSKRPS

SEQ ID NO: 1047 - protein, 11 aa, Homo sapiens
HTYTTLKPSVV

SEQ ID NO: 1048 - protein, 108 aa, Homo sapiens
SYELTQPPSVSVSPGQTASITCSGDKLGDKYASWYQQKPGQSPVLVIYQDSKRPSGIPERFSGSNSGNTATLTISGTQAEDEADYYCHTYTTLKPSVVFGGGTKLTVL

SEQ ID NO: 1049 - DNA, 360 bp, Homo sapiens; identical to SEQ ID NO: 439

SEQ ID NO: 1050 - DNA, 324 bp, Homo sapiens
agctacgagctgacacagcctcctagcgtttccgtgtctcctggccagacagccagcatcacatgttctggcgacaagctgggcgataagtacgccagctggtatcagcagaagcccggacagtctcccgtgctggtcatctaccaggatagcaagaggcctagcggcatccctgagagattcagcggcagcaatagcggcaataccgccacactgacaatcagcggaacacaggccgaggacgaggccgattactactgtcacacctacaccacactgaagcccagcgtggtgttcggcggcggtaccaagctgacagtgctg

SEQ ID NO: 1051 - protein, 5 aa, Homo sapiens
HAWIS

SEQ ID NO: 1052 - protein, 19 aa, Homo sapiens
QIKGGPGSGGTSYAEPVKG

SEQ ID NO: 1053 - protein, 9 aa, Homo sapiens
LGIYSGFDY

SEQ ID NO: 1054 - protein, 120 aa, Homo sapiens; identical to SEQ ID NO: 844

SEQ ID NO: 1055 - protein, 11 aa, Homo sapiens
SGDKLGDKYAS

SEQ ID NO: 1056 - protein, 7 aa, Homo sapiens
QDSKRPS

SEQ ID NO: 1057 - protein, 11 aa, Homo sapiens
HTYSHRPEIVV

SEQ ID NO: 1058 - protein, 108 aa, Homo sapiens; identical to SEQ ID NO: 968

SEQ ID NO: 1059 - DNA, 360 bp, Homo sapiens; identical to SEQ ID NO: 849

SEQ ID NO: 1060 - DNA, 324 bp, Homo sapiens; identical to SEQ ID NO: 970

SEQ ID NO: 1061 - protein, 5 aa, Homo sapiens
HAWIS

SEQ ID NO: 1062 - protein, 19 aa, Homo sapiens
QIKGGPGSGGTSYAEPVKG

SEQ ID NO: 1063 - protein, 9 aa, Homo sapiens
LGIYSGFDY

SEQ ID NO: 1064 - protein, 120 aa, Homo sapiens; identical to SEQ ID NO: 844

SEQ ID NO: 1065 - protein, 11 aa, Homo sapiens
SGDKLGDKYAS

SEQ ID NO: 1066 - protein, 7 aa, Homo sapiens
QDSKRPS

SEQ ID NO: 1067 - protein, 11 aa, Homo sapiens
HSYLHLPPTVV

SEQ ID NO: 1068 - protein, 108 aa, Homo sapiens; identical to SEQ ID NO: 1018

SEQ ID NO: 1069 - DNA, 360 bp, Homo sapiens; identical to SEQ ID NO: 849

SEQ ID NO: 1070 - DNA, 324 bp, Homo sapiens; identical to SEQ ID NO: 970